(19) United States
(12) Patent Application Publication
(10) Pub. No.: US 2025/0026838 A1
(43) Pub. Date: Jan. 23, 2025

(54) METHODS OF PREVENTING PROTEIN AGGREGATION

(71) Applicant: TrueBinding, Inc., Foster City, CA (US)

(72) Inventors: Dongxu Sun, Los Altos, CA (US); Suhail Rasool, Foster City, CA (US); Ragadeepthi Tunduguru, Foster City, CA (US); Diksha Bhatia, Mountain View, CA (US); Vishal Ramahari Mali, Fremont, CA (US); Mandakini J. Patel, San Antonio, TX (US); Yew Ann Leong, San Francisco, CA (US); Dalya Rivka Rosner, Redwood City, CA (US); Ksenya Shchors, San Mateo, CA (US); Ke Hong, Saratoga, CA (US)

(21) Appl. No.: 18/578,450

(22) PCT Filed: Jul. 13, 2022
(86) PCT No.: PCT/US2022/073694

§ 371 Date: Jan. 11, 2024

Related U.S. Application Data
(60) Provisional application No. 63/263,622, filed on Nov. 5, 2021.
(60) Provisional application No. 63/221,395, filed on Jul. 13, 2021.

(51) Int. Cl. C07K 16/28; A61P 25/28

(52) U.S. Cl. CPC C07K 16/2851; A61P 25/28; C07K 2317/565; C07K 2317/76

(57) ABSTRACT

Disclosed herein are methods and compositions for preventing or inhibiting amyloid complex formation of proteins that form pathological aggregates. Examples of these proteins include α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, and myostatin propeptide. These methods and compositions involve antibodies or binding fragments thereof that bind to galectin-3 (Gal3).

### CROSS-REFERENCE TO RELATED APPLICATIONS

This application claims the benefit of priority of U.S. Provisional Patent Application No. 63/221,395, filed Jul. 13, 2021, and U.S. Provisional Patent Application No. 63/263,622, filed Nov. 5, 2021, each of which is hereby expressly incorporated by reference in its entirety, including any appendices filed therewith.

### REFERENCE TO SEQUENCE LISTING

The present application is being filed along with a Sequence Listing in electronic format. The Sequence Listing is provided as a file entitled IMMUT.031WO.XML, which was created and last modified on Jul. 12, 2022, which is 2,976,441 bytes in size. The information in the electronic Sequence Listing is hereby incorporated by reference in its entirety.

### FIELD

Aspects of the present disclosure relate generally to antibodies or binding fragments thereof that bind to Galectin-3 (Gal3), and methods of using to prevent or inhibit amyloid complex formation of proteins that form pathological aggregates.

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

### SUMMARY

Galectin-3 (Gal3) has been implicated to have immunomodulatory activity. An example of this is the interaction between Gal3 and T-cell immunoglobulin and mucin-domain containing-3 (TIM-3), which causes suppression of immune responses such as T cell activation and may enable cancer cells to evade immune clearance. Antibodies that bind to Gal3 and methods of making and using them are exemplified in WO 2019/023247, WO 2020/160156, and WO 2021/113527, each of which is hereby expressly incorporated by reference in its entirety.

In some embodiments, a method of inhibiting Gal3-mediated amyloid aggregation of a protein is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of the protein.

In some embodiments, a method of inhibiting Gal3-mediated oligomerization of a protein is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of the protein.

In some embodiments, a method of treating an amyloid proteopathy in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of a protein in the subject, thereby treating the amyloid proteopathy in the subject.

In some embodiments, a method of treating a proteopathy in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of a protein in the subject, thereby treating the amyloid proteopathy in the subject.

In some embodiments, a method of promoting amyloid aggregation and/or oligomerization of a protein is disclosed. In some embodiments, the method comprises contacting the protein with Gal3, wherein Gal3 promotes amyloid aggregation and/or oligomerization of the protein.

In some embodiments, a composition comprising a protein and Gal3, wherein Gal3 promotes amyloid aggregation and/or oligomerization of the protein is disclosed.

In some embodiments, a method of inhibiting Gal3-mediated amyloid aggregation of amyloid β40 and/or amyloid β42 is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of amyloid β40 and/or amyloid β42.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of amyloid β40 and/or amyloid β42 in the subject, thereby treating Alzheimer's disease in the subject.

In some embodiments, a method of treating CAA in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated aggregation of amyloid β40 and/or amyloid β42 in the subject, thereby treating CAA in the subject.

In some embodiments, a method of inhibiting Gal3-mediated amyloid aggregation of phospho tau is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of phospho tau.

In some embodiments, a method of treating Alzheimer's disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of phospho tau in the subject, thereby treating Alzheimer's disease in the subject.

In some embodiments, a method of treating tauopathies in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of phospho tau in the subject, thereby treating the tauopathy in the subject.

In some embodiments, a method of inhibiting Gal3-mediated oligomerization of alpha synuclein is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of alpha synuclein.

In some embodiments, a method of treating Lewy body disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of alpha synuclein in the subject, thereby treating Lewy body disease in the subject.

In some embodiments, a method of treating multiple system atrophy in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of alpha synuclein in the subject, thereby treating multiple system atrophy in the subject.

In some embodiments, a method of inhibiting Gal3-mediated oligomerization of APOE-4 is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of APOE-4.

In some embodiments, a method of treating Alzheimer's disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of APOE-4 in the subject, thereby treating Alzheimer's disease in the subject.

In some embodiments, a method of treating CAA in a subject in need thereof is disclosed. In some embodiments the method comprises:administering to the subject an anti-Gal3 antibody or binding fragment thereof, wherein binding of the anti-Gal3 antibody or binding fragment thereof to Gal3 in the subject inhibits Gal3-mediated oligomerization of alpha synuclein in the subject, thereby treating CAA in the subject.

In some embodiments, a method of inhibiting Gal3-mediated oligomerization of cholesterol is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of cholesterol.

In some embodiments, a method of treating Alzheimer's disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cholesterol in the subject, thereby treating Alzheimer's disease in the subject.

In some embodiments, a method of treating cardiovascular disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cholesterol in the subject, thereby treating cardiovascular disease in the subject.

In some embodiments, a method of treating atherosclerosis disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cholesterol in the subject, thereby treating atherosclerosis in the subject.

In some embodiments, a method of inhibiting Gal3-mediated oligomerization of cholesteryl is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of cholesteryl.

In some embodiments, a method of treating Alzheimer's disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cholesteryl in the subject, thereby treating Alzheimer's disease in the subject.

In some embodiments, a method of treating cardiovascular disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cholesteryl in the subject, thereby treating cardiovascular disease in the subject.

In some embodiments, a method of treating atherosclerosis disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cholesteryl in the subject, thereby treating atherosclerosis in the subject.

In some embodiments, a method of inhibiting Gal3-mediated oligomerization of neuroserpin is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of neuroserpin.

In some embodiments, a method of treating familial encephalopathy with neuroserpin inclusion bodies (FENIB) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of neuroserpin in the subject, thereby treating familial encephalopathy with neuroserpin inclusion bodies (FENIB) in the subject.

In some embodiments, a method of inhibiting Gal3-mediated oligomerization of insulin is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of insulin.

In some embodiments, a method of treating insulin-derived amyloidosis in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of insulin in the subject, thereby treating insulin-derived amyloidosis in the subject.

In some embodiments, a method of treating diabetes in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of insulin in the subject, thereby treating diabetes in the subject.

In some embodiments, a method of inhibiting Gal3-mediated oligomerization of cystatin-c is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of cystatin-c.

In some embodiments, a method of treating Alzheimer's disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cystatin-c in the subject, thereby treating Alzheimer's disease in the subject.

In some embodiments, a method of treating CAA in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cystatin-c in the subject, thereby treating CAA in the subject.

In some embodiments, a method of treating kidney disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of cystatin-c in the subject, thereby treating kidney disease in the subject.

In some embodiments, a method of inhibiting Gal3-mediated oligomerization of prion protein is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of prion protein.

In some embodiments, a method of treating prion disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of prion protein in the subject, thereby treating prion disease in the subject.

In some embodiments, a method of treating transmissible spongiform encephalopathy (TSE) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of prion protein in the subject, thereby treating transmissible spongiform encephalopathy (TSE) in the subject.

In some embodiments, a method of treating familial Creutzfeldt-Jakob disease (CJD) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of prion protein in the subject, thereby treating familial Creutzfeldt-Jakob disease (CJD) in the subject.

In some embodiments, a method of treating fatal familial insomnia in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of prion protein in the subject, thereby treating fatal familial insomnia in the subject.

In some embodiments, a method of treating Gerstmann-Straussler-Scheinker disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of prion protein in the subject, thereby treating Gerstmann-Straussler-Scheinker disease in the subject.

In some embodiments, a method of inhibiting Gal3-mediated oligomerization of myostatin is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of myostatin.

In some embodiments, a method of treating idiopathic inflammatory myopathies (IIM) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of myostatin in the subject, thereby treating idiopathic inflammatory myopathies (IIM) in the subject.

In some embodiments, a method of inhibiting Gal3-mediated oligomerization of transthyretin is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of transthyretin.

In some embodiments, a method of treating transthyretin amyloidosis in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of transthyretin in the subject, thereby treating transthyretin amyloidosis in the subject.

In some embodiments, a method of treating heart and/or kidney disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of transthyretin in the subject, thereby treating heart and/or kidney disease in the subject.

In some embodiments, a method of treating preeclampsia in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of transthyretin in the subject, thereby treating preeclampsia in the subject.

In some embodiments, a method of inhibiting Gal3-mediated oligomerization of phenylalanine is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of phenylalanine.

In some embodiments, a method of treating phenylketonuria in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of phenylalanine in the subject, thereby treating phenylketonuria in the subject.

In some embodiments, a method of inhibiting Gal3-mediated oligomerization of glutamine is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of glutamine.

In some embodiments, a method of treating Huntington Disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of glutamine in the subject, thereby treating Huntington Disease in the subject.

In some embodiments, a method of inhibiting Gal3-mediated oligomerization of Neurofibrillary Light chain (NFL) is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of NFL.

In some embodiments, a method of treating motor neuron degeneration in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of NFL in the subject, thereby treating motor neuron degeneration in the subject.

In some embodiments, a method of inhibiting Gal3-mediated amyloid aggregation of fibrin is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of fibrin.

In some embodiments, a method of treating cerebrovascular damage in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of fibrin in the subject, thereby treating cerebrovascular damage in the subject.

In some embodiments, a method of treating stroke in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of fibrin in the subject, thereby treating stroke in the subject.

In some embodiments, a method of treating CAA in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of fibrin in the subject, thereby treating CAA in the subject.

In some embodiments, a method of treating Alzheimer's disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of fibrin in the subject, thereby treating Alzheimer's disease in the subject.

In some embodiments, a method of inhibiting Gal3-mediated oligomerization of lysozyme is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of lysozyme.

In some embodiments, a method of treating human systemic amyloid disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of lysozyme in the subject, thereby treating human systemic amyloid disease in the subject.

In some embodiments, a method of inhibiting Gal3-mediated amyloid aggregation of complement proteins C3 and/or C9 is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of complement proteins C3 and/or C9.

In some embodiments, a method of treating disruption in innate immune system in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of complement proteins C3 and/or C9 in the subject, thereby treating disruption in innate immune system in the subject.

In some embodiments, a method of inhibiting Gal3-mediated oligomerization of crystallins is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of crystallins.

In some embodiments, a method of treating damage to lens of a subject's eye and/or blurring of vision in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of crystallins in the subject, thereby treating damage to lenses of the subject's eye and/or blurring of vision in the subject.

In some embodiments, a method of inhibiting Gal3-mediated oligomerization of atrial natriuretic peptide (ANP) is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of ANP.

In some embodiments, a method of treating congestive heart failure (CHF) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of ANP in the subject, thereby treating CHF in the subject.

In some embodiments, a method of treating cardiac amyloidosis in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of ANP in the subject, thereby treating cardiac amyloidosis in the subject.

In some embodiments, a method of inhibiting Gal3-mediated oligomerization of B-Type Natriuretic Peptide (BNP) is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of BNP.

In some embodiments, a method of treating congestive heart failure (CHF) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of BNP in the subject, thereby treating CHF in the subject.

In some embodiments, a method of treating cardiac amyloidosis in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of BNP in the subject, thereby treating cardiac amyloidosis in the subject.

In some embodiments, a method of inhibiting Gal3-mediated oligomerization calcitonin is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated amyloid aggregation of calcitonin.

In some embodiments, a method of treating medullary carcinoma of the thyroid (MTC) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of calcitonin in the subject, thereby treating MTC in the subject.

In some embodiments, a method of treating osteoporosis in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of calcitonin in the subject, thereby treating osteoporosis in the subject.

In some embodiments, a method of treating Paget's Disease in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of calcitonin in the subject, thereby treating Paget's Disease in the subject.

In some embodiments, a method of inhibiting Gal3-mediated oligomerization of Serum Amyloid (A) (SAA) is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of Serum Amyloid (A) (SAA).

In some embodiments, a method of treating peripheral amyloidosis in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of Serum Amyloid (A) (SAA) in the subject, thereby treating peripheral amyloidosis in the subject.

In some embodiments, a method of inhibiting Gal3-mediated amyloid aggregation of islet amyloid polypeptide (IAPP) is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of IAPP.

In some embodiments, a method of treating type 2 diabetes in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of IAPP in the subject, thereby treating type 2 diabetes in the subject.

In some embodiments, a method of inhibiting Gal3-mediated amyloid aggregation of TAR DNA binding protein 43 (TDP-43) is disclosed. In some embodiments the method comprises: contacting the protein with an anti-Gal3 antibody or binding fragment thereof, wherein binding of the anti-Gal3 antibody or binding fragment thereof to Gal3 inhibits Gal3-mediated oligomerization of TDP-43.

In some embodiments, a method of treating amyotrophic lateral sclerosis (ALS) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of TDP-43 in the subject, thereby treating ALS in the subject.

In some embodiments, a method of treating frontotemporal lobar degeneration (FTLD) in a subject in need thereof is disclosed. In some embodiments the method comprises: administering to the subject an anti-Gal3 antibody or binding fragment thereof, wherein binding of the anti-Gal3 antibody or binding fragment thereof to Gal3 in the subject inhibits Gal3-mediated oligomerization of TDP-43 in the subject, thereby treating FTLD in the subject.

In some embodiments, a method of inhibiting Gal3-mediated oligomerization is disclosed. In some embodiments, the method comprises: contacting one or more monomers with an anti-Gal3 antibody or binding fragment thereof, wherein binding of the anti-Gal3 antibody or binding fragment thereof to Gal3 inhibits Gal3-mediated oligomerization of the pone or more monomers.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 is a flow chart depicting embodiments of a method of inhibiting Gal3-mediated protein aggregation.

FIG. 2 is a flow chart depicting embodiments of a method of promoting Gal3-mediated protein aggregation.

FIG. 3 is a flow chart depicting embodiments of a method of treating amyloid proteopathy in a subject.

FIG. 4A-C depict promotion of aggregation of α-synuclein by Gal3. FIG. 4A shows a Western blot of α-synuclein incubated with or without Gal3 over 0-5 hours. FIG. 4B shows quantification of α-synuclein aggregation with Gal3 over the 0-5 hour time points. FIG. 4C shows dot blots showing oligomerization of α-synuclein when incubated with Gal3.

FIG. 5A-F depict promotion of aggregation of Tau protein by Gal3. FIG. 5A shows a Western blot of Tau protein incubated with or without Gal3 at 0 and 5 hour time points. FIG. 5B shows quantification of Tau protein dimerization and trimerization with or without incubation with or without Gal3 at 0 and 5 hour time points. FIG. 5C shows dot blots showing that non-phosphorylated Tau oligomerizes very lightly when mixed with Gal3. FIG. 5D shows dot blots showing that phosphorylated Tau (phospho-tau (S396)) is dramatically oligomerized when mixed with Gal3. FIG. 5E and FIG. 5F shows time course aggregation of 4RTau (FIG. 5E) and Phospho Tau (FIG. 5F) (0.1 mg/mL) incubated with 100 μg of rhGalectin-3 probed with A11, Total Tau (Tau 5), PhosTau (Ser396) and Galectin-3 (804)Ab.

FIG. 6 depicts a dot blot of TDP-43 aggregation incubated with or without Gal3 as detected with antibody A11, anti-TDP-43 antibody, and anti-Gal3 antibody.

FIG. 7A-C depict promotion of aggregation of TDP-43 by Gal3. FIG. 7A shows a Western blot of TDP-43 incubated with or without Gal3 over 0-5 hours detected with anti-Gal3 antibody. FIG. 7B shows a Western blot of TDP-43 incubated with or without Gal3 over 0-5 hours detected with anti-TDP-43 antibody, showing multimerization of TDP-43. FIG. 7C depicts quantification of TDP-43 aggregation with or without Gal3 over the 0-5 hour time points.

FIG. 8A-D depicts promotion of TTR aggregation by Gal3. FIG. 8A shows a Western blot of TTR incubated with or without Gal3 over 0-24 hours detected with anti-TTR antibody. FIG. 8B depicts quantification of TTR aggregation with or without Gal3 over the 0-24 hour time points. FIG. 8C shows a Western blot of TTR incubated with or without Gal3 over 1-6 day time points. FIG. 8D depicts quantification of TTR aggregation with or without Gal3 over the 1-6 day time points.

FIG. 9A-D depicts promotion of IAPP aggregation by Gal3. FIG. 9A depicts a Western blot of IAPP alone, IAPP mixed with Gal3, and Gal3 alone, showing the presence of a ˜60 kDa band in the IAPP alone condition, and intensification of the band in the IAPP+Gal3 condition. FIG. 9B depicts quantification of the intensity of the bands of FIG. 9A reflecting protein aggregates as detected by the I11 antibody. FIG. 9C depicts a Western blot of IAPP alone, IAPP mixed with Gal3, and Gal3 alone at time points of 0, 0.5, 3, and 5 hours of incubation, suggesting that IAPP oligomerizes after 3 hours of incubation with Gal3. FIG. 9D is the quantification of the intensity of the IAPP bands of FIG. 9C.

FIG. 10 depicts protein sequences of Gal3 and exemplary proteins that exhibit pathogenic aggregation.

FIG. 11 depicts peptide sequences of Gal3 used to generate and analyze antibodies.

FIG. 12A depicts exemplary variable heavy chain complementarity-determining region (CDR) 1 for anti-Gal3 antibodies disclosed herein. In some embodiments, any of the compositions or methods provided herein can include one or more of the variable heavy chain CDR1 provided herein.

FIG. 12B depicts exemplary variable heavy chain CDR2 for anti-Gal3 antibodies disclosed herein. In some embodiments, any of the compositions or methods provided herein can include one or more of the variable heavy chain CDR2 provided herein.

FIG. 12C depicts exemplary variable heavy chain CDR3 for anti-Gal3 antibodies disclosed herein. In some embodiments, any of the compositions or methods provided herein can include one or more of the variable heavy chain CDR3 provided herein.

FIG. 13A depicts exemplary variable light chain CDR1 for anti-Gal3 antibodies disclosed herein. In some embodiments, any of the compositions or methods provided herein can include one or more of the variable light chain CDR1 provided herein.

FIG. 13B depicts exemplary variable light chain CDR2 for anti-Gal3 antibodies disclosed herein. In some embodiments, any of the compositions or methods provided herein can include one or more of the variable light chain CDR2 provided herein.

FIG. 13C depicts exemplary variable light chain CDR3 for anti-Gal3 antibodies disclosed herein. In some embodiments, any of the compositions or methods provided herein can include one or more of the variable light chain CDR3 provided herein.

FIG. 14 depicts exemplary heavy chain variable region (VH) sequences for anti-Gal3 antibodies disclosed herein. In some embodiments, any of the compositions or methods provided herein can include one or more of the VH sequences provided herein.

FIG. 15 depicts exemplary light chain variable region (VL) sequences for anti-Gal3 antibodies disclosed herein. In some embodiments, any of the compositions or methods provided herein can include one or more of the VL sequences provided herein.

FIG. 16 depicts exemplary combinations of heavy and light chain CDRs (CDR1, CDR2, and CDR3) of exemplary anti-Gal3 antibodies disclosed herein. In some embodiments, any of the compositions or methods provided herein can include one or more of the heavy and light chain CDR combinations provided herein.

FIG. 17 depicts exemplary combinations of heavy and light chain variable regions of exemplary anti-Gal3 antibodies disclosed herein. In some embodiments, any of the compositions or methods provided herein can include one or more of the heavy and light chain variable region combinations provided herein.

FIG. 18 depicts exemplary heavy chain (HC) sequences and light chain (LC) sequences, and possible pairings for exemplary anti-Gal3 antibodies disclosed herein. In some embodiments, any of the compositions or methods provided herein can include one or more of the HC or LC, or pairs of HC and LC sequences provided herein.

FIG. 19 depicts antibody names used throughout the present disclosure refer to the same antibody (with exemplary peptide and nucleic acid sequences provided elsewhere in the disclosure and appropriately attributed to at least one of the depicted names) and may be used interchangeably. The names shown in a column correspond to the same antibody.

FIG. 20 depicts an alignment of hinge and constant heavy chain domain 2 (C_{H}2) domain amino acid sequences of wild-type human immunoglobulin G1 (IgG1), IgG2 and IgG4 as well as their sigma variants. The alignment above uses EU numbering. Residues identical to wild-type IgG1 are indicated as dots; gaps are indicated with hyphens. Sequence is given explicitly if it differs from wild-type IgG1 or from the parental subtype for a variants. Open boxes beneath the alignment correspond to International Immunogenetics Information System (IMGT) strand definitions. Boxes beneath the alignment correspond to the strand and helix secondary structure assignment for wild-type IgG1. Residues 267-273 form the BC loop and 322-332 form the FG loop. Also provided are exemplary constant regions for human IgG4 heavy (S228P mutant) and light (kappa) chains (SEQ ID NOs: 931-932) and murine IgG2A (LALAPG and LALA mutants) (SEQ ID NOs: 933-934). In some embodiments, any one or more of the VH/VL and/or CDRs provided in the other figures or otherwise disclosed herein can be paired with any one or more of the exemplary constant regions provided herein.

FIG. 21 depicts nucleic acid sequences that encode for exemplary heavy chain variable regions of anti-Gal3 antibodies disclosed herein. In some embodiments, any of the compositions or methods provided herein can include one or more of the heavy chain variable regions encoded by the nucleic acids provided herein.

FIG. 22 depicts nucleic acid sequences that encode for exemplary light chain variable regions of anti-Gal3 antibodies disclosed herein. In some embodiments, any of the compositions or methods provided herein can include one or more of the light chain variable regions encoded by the nucleic acids provided herein.

FIG. 23 depicts nucleic acid sequences that encode for exemplary heavy chains of anti-Gal3 antibodies disclosed herein. In some embodiments, any of the compositions or methods provided herein can include one or more of the heavy chains encoded by the nucleic acids provided herein.

FIG. 24 depicts nucleic acid sequences that encode for exemplary light chains of anti-Gal3 antibodies disclosed herein. In some embodiments, any of the compositions or methods provided herein can include one or more of the light chains encoded by the nucleic acids provided herein.

FIG. 25A-B depicts an exemplary alignment for the heavy chain CDRs (FIG. 25A) and light chain CDRs (FIG. 25B) for the exemplary anti-Gal3 antibodies disclosed herein.

FIG. 26A-C depict promotion of aggregation of certain polymorphic alleles of apolipoprotein E (APOE) by Gal3. FIG. 26A shows dot blots of APO-E2 mixed with Gal3. FIG. 26B shows dot blots of APO-E3 mixed with Gal3. Neither APO-E2 or APO-E3 showed significant oligomerization when mixed with Gal3. FIG. 26C shows dot blots of APO-E4 mixed with Gal3, showing that APO-E4 is oligomerized when mixed with Gal3.

FIG. 27A-C depict degradation of APOE-4 oligomers by exemplary anti-Gal3 antibody TB006 by dot blot. FIG. 27A shows that there is a dose-dependent ability of TB006 to degrade APO-E4 oligomers. FIG. 27B shows the 3 hour antibody incubation time point for FIG. 27A. FIG. 27C is the quantification of the TB006-treated conditions of FIG. 27B.

FIG. 28A-C depict promotion of aggregation of prion protein (PrP) by Gal3 as detected by dot blot. FIG. 28A depicts time course aggregation over 5 hours of r-Prion protein incubated with 100 μg of Gal-3 probed with A11 antibody. FIG. 28B depicts time course aggregation over 5 hours of r-Prion protein incubated with 100 μg of Gal-3 probed with A11 antibody and re-probed with r-Prion mouse antibody. FIG. 28C depicts time course aggregation over 1 hour of r-Prion protein incubated with 100 μg of Gal-3 probed with (A11) antibody and re-probed with r-Prion mouse antibody.

FIG. 29A-B depicts promotion of aggregation of neurofilament light (NFL) protein by Gal3 as detected by dot blot.

FIG. 30A-C depict promotion of aggregation of Aβ40 by Gal3 as detected by dot blot. FIG. 30A-C shows 24 hour time course aggregation of Aβ40 incubated with 100 μg of Gal-3 probed with A11 (FIG. 30A), 6E10 (Biolegend, catalog #SIG-39300) (FIG. 30B), and 804 (QC 190207)(FIG. 30C) antibodies.

FIG. 31A-D depict degradation of toxic Aβ42 oligomers as detected by dot blot. FIG. 31A depicts 24 hour time course degradation of toxic Aβ42 oligomers by hTB006 probed with A11 and 6E10 antibodies. FIG. 31B depicts quantification of Aβ42 oligomer degradation over the 24 hour time course. FIG. 31C depicts 5 hour time course degradation of toxic Aβ42 oligomers by hTB006 probed with A11 and 6E10 antibodies. FIG. 31D depicts quantification of the effect of different concentrations of hTB006 on Gal-3 induced Aβ42 oligomers.

FIG. 32A-F depict time course aggregation of Fibrin incubated with 100 μg of Gal-3 probed with A11 antibody as detected by dot blot. FIG. 32A depicts time course aggregation of Fibrin incubated with 100 μg of Gal-3 probed with A11 antibody as detected by dot blot. FIG. 32B depicts quantification of Gal-3 intrinsic promotion of Fibrin oligomerization. FIG. 32C depicts degradation of toxic Fibrin oligomers by TB001 and TB006 as detected by dot blot. FIG. 32D depicts screening of different Gal-3 antibody clones on Fibrin oligomerization probed with A11 antibody. FIG. 32E depicts quantification of different Gal-3 antibody clones on Fibrin oligomerization probed with A11 antibody. FIG. 32F is a table depicting the identity and isotype of Gal-3 antibody clones screened.

FIG. 33 depicts time course aggregation of CRP and SUMO incubated with 100 μg of Gal-3 probed with A11 antibody as detected by dot blot.

FIG. 34A-C depict time course aggregation of Light Chain, PDGFR, and MCAM, incubated with 100 μg of Gal-3 probed with A11 antibody as detected by dot blot. FIG. 34A depicts time course aggregation of Light Chain incubated with 100 μg of Gal-3 probed with A11 antibody as detected by dot blot. FIG. 34B depicts time course aggregation of PDGFR incubated with 100 μg of Gal-3 probed with A11 antibody as detected by dot blot. FIG. 34C depicts time course aggregation of MCAM incubated with 100 μg of Gal-3 probed with A11 antibody as detected by dot blot.

FIG. 35A-B depict 24 hour time course aggregation of complement proteins (C3 & C9) incubated with 100 μg of Gal-3 probed with A11 antibody as detected by dot blot. FIG. 35A depicts 24 hour time course aggregation of complement proteins C3 incubated with 100 μg of Gal-3 probed with A11 antibody as detected by dot blot. FIG. 35B depicts 24 hour time course aggregation of complement protein C9 incubated with 100 μg of Gal-3 probed with A11 antibody as detected by dot blot.

FIG. 36 depicts 24 hour time course aggregation of lysozyme incubated with 100 μg of Gal-3 probed with A11 antibody as detected by dot blot.

FIG. 37 depicts 4 hour time course aggregation of insulin incubated at 50 C with 100 μg and 200 μg of Gal-3 probed with A11 antibody as detected by dot blot.

FIG. 38A-B depicts 5 hour time course aggregation of native haemoglobin (Hb) and glycosylated haemoglobin (HbAIC) incubated at room temperature (RT) and 37 C with 100 μg of Gal-3 probed with A11 antibody as detected by dot blot. FIG. 38A depicts 5 hour time course aggregation of native haemoglobin (Hb) incubated at room temperature (RT) and 37 C with 100 μg of Gal-3 probed with A11 antibody as detected by dot blot. FIG. 38B depicts 5 hour time course aggregation of glycosylated haemoglobin (HbAIC) incubated at room temperature (RT) and 37 C with 100 μg of Gal-3 probed with A11 antibody as detected by dot blot.

FIG. 39A-B depict 5 hour time course aggregation of phenylalanine (Phe) incubated at room temperature (RT) and 37 C with 100 μg of Gal-3 probed with A11 antibody as detected by dot blot.

FIG. 40 depicts 5 hour time course aggregation of glutamine (GLN) incubated at room temperature (RT) with 100 μg of Gal-3 probed with A11 antibody as detected by dot blot.

FIG. 41 depicts 5 hour time course aggregation of cholesteryl (Co-Esteryl) incubated at room temperature (RT) with 100 μg of Gal-3 probed with A11 antibody as detected by dot blot.

FIG. 42A-B depicts 5 hour time course aggregation of cholesterol incubated at room temperature (RT) with 100 μg of Gal-3 probed with antibody as detected by dot blot.

FIG. 43A-B depicts aggregation of neuroserpin incubated at room temperature (RT) with 100 μg of Gal-3 probed with antibody. FIG. 43A depicts 5 hour time course aggregation of neuroserpin as detected by dot blot. FIG. 43B depicts visualization of neuroserpin aggregation with and without Gal-3 as detected using fluorescent microscopy.

FIG. 44A-B depict comparative degradation of Aβ42 oligomers by TB139 and TB006 incubated at RT and probed with oligomer A11 6E10 degrading antibody as detected by dot blot. FIG. 44A depicts comparative degradation of Aβ42 oligomers by TB139 and TB006 incubated at RT and probed with oligomer A11 degrading antibody as detected by dot blot. FIG. 44B depicts comparative degradation of Aβ42 oligomers by TB139 and TB006 incubated at RT and probed with 6E10 degrading antibody as detected by dot blot.

FIG. 45A-B depicts time course aggregation of Crystallin AA incubated with 100 μg of Gal-3 probed with A11 antibody. FIG. 45A depicts visualization of Crystallin AA aggregation with and without Gal-3 as detected using fluorescent microscopy. FIG. 45B depicts time course aggregation of Crystallin AA incubated with 100 μg of Gal-3 probed with A11 antibody as detected by dot blot.

FIG. 46A-B depicts time course aggregation of Crystallin AA incubated with 100 μg of Gal-3 probed with A11 antibody. FIG. 46A depicts visualization of Crystallin AB aggregation with and without Gal-3 as detected using fluorescent microscopy. FIG. 46B depicts time course aggregation of Crystallin AB incubated with 100 μg of Gal-3 probed with A11 antibody as detected by dot blot.

FIG. 47A-F depicts time course aggregation of Cystatin-C incubated with 100 μg of Gal-3 probed with A11 antibody as detected by dot blot. FIG. 47A depicts 24 hour time course aggregation of Cystatin-C incubated with 100 μg of Gal-3 probed with A11 antibody as detected by dot blot. FIG. 47B depicts 24 hour time course aggregation of Cystatin-C incubated with 100 μg of Gal-3 probed with Cystatin-C antibody as detected by dot blot. FIG. 47C depicts 24 hour time course aggregation of Cystatin-C incubated with 100 μg of Gal-3 probed with 804 antibody as detected by dot blot. FIG. 47D depicts 5 hour time course aggregation of Cystatin-C incubated with Gal-3 probed with A11 antibody as detected by dot blot. FIG. 47E depicts 5 hour time course aggregation of Cystatin-C incubated with Gal-3 probed with cystatin-C antibody as detected by dot blot. FIG. 47F depicts 5 hour time course aggregation of Cystatin-C incubated with Gal-3 probed with 804 antibody as detected by dot blot.

FIG. 48A-C depicts 24 hour time course aggregation of Myostatin pro-peptide incubated with 100 μg of Gal-3 probed with A11 antibody. FIG. 48A depicts 24 hour time course aggregation of Myostatin pro-peptide incubated with 100 μg of Gal-3 probed with A11 antibody as detected by dot blot. FIG. 48B-C depict visualization of myostatin pro-peptide aggregation at 5, 24, and 48 hours with and without Gal-3 as detected using fluorescent microscopy.

FIG. 49A-G depicts Insulin oligomerization by Gal-3 and screening of different Gal-3 antibody clones for degradation of insulin oligomerization probed with oligomer degrading A11 antibody. FIG. 49A depicts 3 hour time course aggregation of insulin incubated at 50 C with Gal-3 probed with A11 antibody as detected by dot blot. FIG. 49B depicts aggregation of insulin incubated with Gal-3 for 48 hours and probed with A11 antibody as detected by dot blot. FIG. 49C depicts quantification of insulin aggregation when incubated with Gal-3 for 48 hours and probed with A11 antibody. FIG. 49D depicts a chart depicting the identity and isotype of the Gal-3 antibody clones that were Screened. FIG. 49E depicts visualization of insulin aggregation at three hours with and without Gal-3 as detected using fluorescent microscopy. FIG. 49F depicts 4 hour time course aggregation of Insulin incubated with 100 μg of Gal-3 probed with A11 antibody. FIG. 49F depicts 4 hour time course aggregation of Insulin incubated with 100 μg of Gal-3 probed with anti-Gal3 antibody.

FIG. 50 depicts a 48-hour time course of recombinant human calcitonin (hCT) aggregation when incubated with 100 μg of Gal-3 probed with A11 antibody as detected by dot blot.

FIG. 51A-B depicts Gal-3 promotion of Atrial Natriuretic Peptide (ANP) aggregation. FIG. 51A depicts embodiments of an 80-hour time course of ANP aggregation when incubated with 100 μg of Gal-3 probed with A11 antibody as detected by dot blot. FIG. 51B depicts embodiments of fluorescent microscopic visualization of ANP aggregation when incubated with 100 μg of Gal-3 probed with A11 antibody.

FIG. 52A-B depicts Gal-3 promotion of Pro-B type Natriuretic Peptide (BNP) aggregation. FIG. 52A depicts embodiments of a 48-hour time course of BNP aggregation when incubated with 100 μg of Gal-3 probed with A11 antibody as detected by dot blot. FIG. 52B depicts embodiments of fluorescent microscopic visualization of BNP aggregation when incubated with 100 μg of Gal-3 probed with A11 antibody and incubated for 24-hours at room temperature.

FIG. 53 depicts embodiments of a 72-hour time course of Serum Amyloid A (SAA1) aggregation when incubated with 100 μg of Gal-3 probed with A11 antibody as detected by dot blot.

FIG. 54A-B depicts embodiments of Islet Amyloid Polypeptide (IAPP) when incubated with or without Gal-3. FIG. 54A depicts embodiments of a 24-hour time course of IAPP aggregation when incubated with 100 μg of Gal-3 probed with A11 antibody as detected by dot blot. FIG. 54B depicts embodiments of fluorescent microscopic visualization of IAPP aggregation when incubated with 100 μg of Gal-3 probed with A11 antibody.

FIG. 55A-F depicts embodiments of TDP43 aggregation when incubated with or without Gal-3. FIG. 55A depicts embodiments of a 24-hour time course of TDP43 aggregation when incubated with 100 μg of Gal-3 probed with A11 antibody. FIG. 55B depicts embodiments of a 24-hour time course of TDP43 aggregation when incubated with 100 μg of Gal-3 probed with TDP43 antibody. FIG. 55C depicts embodiments of a 24-hour time course of TDP43 aggregation when incubated with 100 μg of Gal-3 probed with Gal-3 antibody. FIG. 55D depicts a 5-hour time course aggregation profile of TDP43 with and without rhGal-3 probed with TDP43 antibody. FIG. 55E depicts some embodiments of quantification of a 5-hour time course aggregation profile of TDP43 with and without rhGal-3 probed with TDP43 antibody. FIG. 55F depicts a 5-day time course aggregation profile of TDP43 with and without rhGal-3 probed with TDP43 antibody.

FIG. 56 is an illustrative representation of some embodiments depicting alternative pathways and assembled states of amyloid.

FIG. 57 is a flow chart depicting some embodiments of methods for inhibiting Gal3-mediated aggregation of a protein.

FIG. 58 is a flow chart depicting embodiments of a method of treating amyloid proteopathy in a subject.

FIG. 59 depicts some embodiments of a polypeptide sequence encoding hGal3 and epitope peptides used for epitope mapping analysis of TB006, TB101 and 2D10 Ab binding with Gal 3 peptides by Microarray & ELISA.

FIG. 60 depicts some embodiments of mutational analysis of TB006 Ab binding with Gal 3 peptides by Microarray.

FIG. 61 depicts some embodiments of mutational analysis of TB101 Ab binding with Gal 3 peptides by Microarray.

FIG. 62 depicts some embodiments of mutational analysis of 2D10 Ab binding with Gal 3 peptides by Microarray.

FIG. 63 depicts some embodiments of mutational analysis of TB006, TB101, 2D10 Ab binding with Gal 3 peptides by Microarray.

FIG. 64 is an illustrative embodiment showing a comparison of some embodiments of epitope mapping analysis.

FIG. 65 is an illustrative embodiment showing a comparison of some embodiments of epitope mapping analysis. Chain A represents the amino acid numbers on the 18 aa peptide whereas Chain B represents the amino acid numbers on the Heavy chain of TB006 FAb and Chain L represents the light chain.

FIG. 66A-k depict some illustrative embodiments of a heat map showing the affinity of anti-Gal3 blocking antibodies to various Gal3 residues. FIG. 66A is a table listing some illustrative embodiments of anti-Gal3 blocking antibodies. FIG. 66B-66K are illustrative heat maps showing the affinity of some embodiments of anti-Gal3 blocking antibodies to Gal3 epitopes.

FIG. 67 is a table depicting the ability of 33 different anti-Gal3 blocking antibodies to block binding of hTB001, hTB006, Aβ42, Aβ40, Aβ42 alpha synuclein, and hTau to Gal3. That is, these antibodies compete for binding to Gal3 with the above proteins.

FIG. 68 is a table depicting the ability of 33 different anti-Gal3 blocking antibodies to block binding of hTB006as to Gal3 measured by Elisa. That is, these antibodies compete for binding to Gal3 with the above proteins.

FIG. 69 depicts the results of an ELISA assay examining binding of hGal3 to aggregated prion protein.

FIG. 70 depicts the results of an ELISA assay examining binding of hGal3 to aggregated Aβ40.

FIG. 71 depicts the results of an ELISA blocking assay.

FIG. 72 is a table quantifying the effect of various anti-Gal3 antibodies at blocking hGal3 binding to Aβ40.

FIG. 73 depicts the results of the ELISA assay examining binding of hGal3 to aggregated phospho-Tau.

FIG. 74 is a table depicting the quantified results of an ELISA screen.

FIG. 75 is a table depicting the quantified results of an ELISA screen.

FIG. 76 depicts the results of an ELISA assay examining binding of hGal3 to aggregated prion protein.

FIG. 77 depicts the results of an ELISA assay examining binding of hGal3 to aggregated cholesterol.

FIG. 78 depicts the results of an ELISA assay examining binding of hGal3 to 5 hour aggregated cholesterol.

FIG. 79 depicts the results of an ELISA assay examining binding of hGal3 to aggregated insulin.

FIG. 80 depicts the results of an ELISA assay examining binding of hGal3 to aggregated prion protein.

FIG. 81 depicts the results of an ELISA assay examining blocking efficacy of various antibodies against binding of hGal3 to aggregated prion protein.

FIG. 82 depicts the results of the ELISA assay examining binding of hGal3 to aggregated NFL.

FIG. 83 depicts the results of the ELISA assay examining binding of hGal3 to aggregated NFL.

FIG. 84 depicts the results of the ELISA assay examining binding of hGal3 to aggregated C3.

FIG. 85 depicts the results of the ELISA assay determining the IC50s of anti-hGal3 antibodies against hGal3:C3.

FIG. 86 depicts the results of the ELISA assay examining binding of hGal3 to aggregated C9.

FIG. 87 depicts the results of the ELISA assay determining the IC50s of anti-hGal3 antibodies against hGal3:C9.

FIG. 88 depicts the results of an ELISA assay examining binding of hGal3 to aggregated and unaggregated ANP.

FIG. 89 depicts the results of an ELISA assay examining binding of hGal3 to various versions of calcitonin with hGal3.

FIG. 90 depicts the results of an ELISA assay examining binding of hGal3 to aggregated and unaggregated IAPP.

FIG. 91 depicts the results of an ELISA assay examining binding of hGal3 to aggregated alpha-synuclein.

FIG. 92 depicts the results of a blocking efficacy determination as measured by Elisa.

FIG. 93 is a table depicting the blocking efficacy of QC200137 IMTAB0172 14H10.2C9-hIgG4(S228P), IMTAB0111 F798-9C.13H12.2F8-hIgG4(S228P), QC200172 IMTAB0196 846.1H12-hIgG4(S228P), TB006 (QC200208), hIgG4 synagis (QC200234)(negative control).

FIG. 94 shows the isolation of TB006 Fab.

FIG. 95 depicts the crystal structure analysis of TB006 Fab and hGal-3 peptide. a-d. Side view (a and b) and top view (c and d) of overall structure of TB006 Fab in complex with hGal3. Light chain and heavy chain of TB006 Fab are illustrated as light pink and pale cyan respectively. CDRs from the light chain and heavy chain are colored in magentas and marine respectively. Gold color ribbon represents the hGal3 peptide. e. Interaction interface of TB006 Fab and hGal3 peptide. f and g. Detailed interaction amino acids between TB006 Fab light chain (f) or heavy chain (g) and hGal3 peptide. The black dash indicates hydrogen bond with distance of 3.1 Å. Interactions not highlighted are hydrophobic interaction.

FIG. 96 depicts a summary of interaction between TB006 Fab and hGal-3 peptide. Dot lines indicate the interactions between CDRs and hGal-3 peptide. Residues highlighted are out of CDR frames.

FIG. 97 depicts reduced GLUT-4 translocation in L-6 cells when with Gal3+insulin aggregates as compared to insulin treatment alone.

### DETAILED DESCRIPTION OF THE DISCLOSURE

Galectin-3 (Gal3, GAL3) is known to play an important role in cell proliferation, adhesion, differentiation, angiogenesis, and apoptosis. This activity is, at least in part, due to immunomodulatory properties and binding affinity towards other immune regulatory proteins, signaling proteins, and other cell surface markers.

Gal3 functions by distinct N-terminal and C-terminal domains. The N-terminal domain (isoform 1: amino acids 1-111, isoform 3: amino acids 1-125) comprise a tandem repeat domain (TRD, isoform 1: amino acids 36-109, isoform 3: amino acids 50-123) and is largely responsible for oligomerization of Gal3. The C-terminal domain (isoform 1: amino acids 112-250, isoform 3: amino acids 126-264) comprise a carbohydrate-recognition-binding domain (CRD), which binds to β-galactosides. An exemplary sequence for isoform 1 of human Gal3 (NCBI Reference No. NP_002297.2) is shown in SEQ ID NO: 1. An exemplary sequence for isoform 3 of human Gal3 (NCBI Reference No. NP_001344607.1) is shown in SEQ ID NO: 2.

As provided herein, Gal3 is shown to promote oligomerization of various proteins, such as α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, or any combination thereof.

The aggregation of these proteins as oligomers may cause a wide range of proteopathies, including but not limited to an amylodiopathy, Alzheimer's disease, cerebral 3-amyloid angiopathy, retinal ganglion cell degeneration in glaucoma, Parkinson's disease, dementia with Lewy bodies, multiple system atrophy, synucleinopathy, Pick's disease, corticobasal degeneration, tauopathy, progressive supranuclear palsy, TDP-43 proteopathy, amyotrophic lateral sclerosis, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, SAA amyloidosis, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, TTR amyloidosis, cardiac amyloidosis, cardiac atrial amyloidosis, uromodulin-associated kidney disease, IAPP amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), pituitary prolactinoma, insulin amyloidosis, corneal lactoferrin amyloidosis, pulmonary alveolar proteinosis, seminal vesicle amyloid, cutaneous lichen amyloidosis, Mallory bodies, odontogenic (Pindborg) tumor amyloid, cancer, aging promoted by amyloid aggregation, or any disease caused by the misfolding or aggregation of proteins, or otherwise known by a person skilled in the art.

Disclosed herein are methods of treating, reducing, ameliorating, or preventing incidence of a proteopathy in a cell or subject by inhibiting the oligomer promoting activity of Gal3. This may be accomplished using an antibody or binding fragment thereof that binds to Gal3.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

As used herein, the term “peptidomimetic” refers to any peptide analog that is able to mimic the structural elements and functionality of natural peptides while also retaining the capability to interact with a biological target and produce the same biological effect as its corresponding natural peptide.

As used herein, the term “oligomer” refers to a molecule that includes a few similar or identical repeating units which could be derived, from copies of a smaller molecule, its monomer. In some embodiments, the oligomer comprises repeating units of a protein monomer. In some embodiments the oligomer comprises repeating units of α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, and/or any combination thereof. In some embodiment, the oligomer is any repeating biological molecule or unit that is known to aggregate into oligomers.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies may be polyclonal antibodies, although monoclonal antibodies may be preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As known in the art, the term “Fc region” is used to define a C-terminal region of an immunoglobulin heavy chain. The “Fe region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1, CDR2, and CDR3 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

As disclosed herein, sequences having a % identity to any of the sequences disclosed herein are envisioned and may be used. The terms “% identity” refer to the percentage of units (i.e. amino acids or nucleotides) that are the same between two or more sequences relative to the length of the sequence. When the two or more sequences being compared are the same length, the % identity will be respective that length. When two or more sequences being compared are different lengths, deletions and/or insertions may be introduced to obtain the best alignment. In some embodiments, these sequences may include peptide sequences, nucleic acid sequences, CDR sequences, variable region sequences, or heavy or light chain sequences. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein may be used. The changes in sequences may apply to, for example, single amino acids, single nucleic acid bases, or nucleic acid codons; however, differences in longer stretches of sequences are also envisioned. As applied to antibody sequences, these differences in sequences may apply to antigen-binding regions (e.g., CDRs) or regions that do not bind to antigens or are only secondary to antigen binding (e.g., framework regions).

As disclosed herein, sequences having a % homology to any of the sequences disclosed herein are envisioned and may be used. The term “% homology” refers to the degree of conservation between two sequences when considering their three-dimensional structure. For example, homology between two protein sequences may be dependent on structural motifs, such as beta strands, alpha helices, and other folds, as well as their distribution throughout the sequence. Homology may be determined through structural determination, either empirically or in silico. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein, which may or may not affect the overall % homology, may be used.

As applied herein, sequences having a certain % similarity to any of the sequence disclosed herein are envisioned and may be used. In some embodiments, these sequences may include peptide sequences, nucleic acid sequences, CDR sequences, variable region sequences, or heavy or light chain sequences. As understood in the art with respect to peptide sequences, “similarity” refers to the comparison of amino acids based on their properties, including but not limited to size, polarity, charge, pK, aromaticity, hydrogen bonding properties, or presence of functional groups (e.g. hydroxyl, thiol, amine, carboxyl, and the like). The term “% similarity” refers to the percentage of units (i.e. amino acids) that are the same between two or more sequences relative to the length of the sequence. When the two or more sequences being compared are the same length, the % similarity will be respective that length. When two or more sequences being compared are different lengths, deletions and/or insertions may be introduced to obtain the best alignment. The similarity of two amino acids may dictate whether a certain substitution is conservative or non-conservative. Methods of determining the conservativeness of an amino acid substitution are generally known in the art and may involve substitution matrices. Commonly used substitution matrices include BLOSUM45, BLOSUM62, BLOSUM80, PAM100, PAM120, PAM160, PAM200, PAM250, but other substitution matrices or approaches may be used as considered appropriate by the skilled person. A certain substitution matrix may be preferential over the others when considering aspects such as stringency, conservation and/or divergence of related sequences (e.g. within the same species or broader), and length of the sequences in question. As used herein, a peptide sequence having a certain % similarity to another sequence will have up to that % of amino acids that are either identical or an acceptable substitution as governed by the method of similarity determination used. In some embodiments, a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 similar substitutions relative to any of the sequences disclosed herein may be used. As applied to antibody sequences, these similar substitutions may apply to antigen-binding regions (i.e. CDRs) or regions that do not bind to antigens or are only secondary to antigen binding (i.e. framework regions).

The term “consensus sequence” as used herein with regard to sequences refers to the generalized sequence representing all of the different combinations of permissible amino acids at each location of a group of sequences. A consensus sequence may provide insight into the conserved regions of related sequences where the unit (e.g. amino acid or nucleotide) is the same in most or all of the sequences, and regions that exhibit divergence between sequences. In the case of antibodies, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a CFD epitope is an antibody that binds this epitope with greater affinity, and/or avidity, and/or more readily, and/or with greater duration than it binds to other CFD epitopes or non-CFD epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “inhibit” refers to the reduction or decrease in an expected activity, such as a cellular activity. The reduction or decrease may be by at least 5%, 10%, 20%, 30%, 40%, 50%, 60%, 70%, 80%, 90%, or 100%, or any percentage that is within a range defined by any two of the aforementioned values, where a reduction or decrease of 100% indicates a complete inhibition and any lower percentage indicates a partial inhibition. The reduction or decrease of the expected activity may be observed in a direct or indirect way.

The term “block” or “disrupt” as used herein with regard to an antibody refers to the ability of an antibody to interfere with a biological process, including but not limited to activity of an enzyme, binding of two or more biological molecules (e.g. two or more proteins, peptides, nucleic acids, lipids, and the like), or advancement of a signaling cascade. Generally, interference with a biological process will involve the antibody binding to its target or an epitope thereof, thereby interfering with the normal function of said target, such as occluding an active site of the target, occluding another region of the target important for its function, or altering the localization and/or transport of the target. The blocking or disruption activity of an antibody may be quantified in terms of the reduction of the biological process in question relative to a control condition where the biological process is not disrupted. In other cases, the blocking or disruption activity of an antibody may be quantified in terms of a modulation in another biological process known to be associated with the target biological process, whether it be directly related or inversely related. In some embodiments, the blocking or disruption activity may cause a change of at least 5%, 10%, 20%, 30%, 40%, 50%, 60%, 70%, 80%, 90%, or 100%, or any percentage within a range defined by any two of the aforementioned values, relative to a control condition. In some embodiments provided herein, an interaction between Gal3 and a protein that forms amyloid aggregates is a biological process that can be disrupted by an anti-Gal3 antibody or binding fragment thereof. It is envisioned that the interaction between Gal3 and the protein that forms amyloid aggregates may or may not be a direct interaction, and the anti-Gal3 antibody or binding fragment thereof may interfere with some other aspect of the activity of Gal3 or the protein that forms amyloid aggregates.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Unless otherwise specified, the complementarity defining regions disclosed herein follow the IMGT definition. In some embodiments, any of the CDRs disclosed herein can instead be interpreted by Kabat, Chothia, or other definitions accepted by those of skill in the art.

The term “humanized” as applies to a non-human (e.g. rodent or primate) antibodies are hybrid immunoglobulins, immunoglobulin chains or fragments thereof which contain minimal sequence derived from non-human immunoglobulin.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

In some non-limiting embodiments, an effective amount or effective dose of a composition or compound may relate to the amount or dose that provides a significant, measurable, or sufficient therapeutic effect towards the treatment of any one or more of the diseases provided herein, such as a synucleinopathy, Parkinson's disease, dementia with Lewy bodies, multiple system atrophy, tauopathy, Alzheimer's disease, progressive supranuclear palsy, corticobasal degeneration, Pick's disease, TDP-43 proteopathy, amyotrophic lateral sclerosis, frontotemporal lobar degeneration, TTR amyloidosis (ATTR), cardiac amyloidosis, uromodulin-associated kidney disease, IAPP amyloidosis, SAA amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation, or any combination thereof. In some embodiments, the effective amount or effective dose of a composition or compound may treat, ameliorate, or prevent the progression of symptoms of any one or more of the diseases provided herein.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the lungs.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, dextran, fructose, mannose, lactose, galactose, sucrose, sorbitol, cellulose, methyl cellulose, hydroxypropyl methyl cellulose (hypromellose), glycerin, polyvinyl alcohol, povidone, propylene glycol, serum, amino acids, polyethylene glycol, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is at least 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

As used herein, the term “proteopathy” refers to a disease which is caused by abnormal folding or accumulation of proteins. An abnormal protein may gain a toxic function, or lose their normal function. It is possible that misfolded proteins can induce the misfolding of otherwise normally folded proteins, resulting in an amplification of the disease (e.g. prion disease). A proteopathy may be an amyloid proteopathy caused by pathogenic accumulation of protein amyloids. Some non-limiting examples of proteopathies include Alzheimer's disease, cerebral β-amyloid angiopathy, retinal ganglion cell degeneration in glaucoma, Parkinson's disease, dementia with Lewy bodies, multiple system atrophy, synucleinopathy, Pick's disease, corticobasal degeneration, tauopathy, progressive supranuclear palsy, TDP-43 proteopathy, amyotrophic lateral sclerosis, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, SAA amyloidosis, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, TTR amyloidosis, cardiac amyloidosis, cardiac atrial amyloidosis, uromodulin-associated kidney disease, IAPP amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), pituitary prolactinoma, insulin amyloidosis, corneal lactoferrin amyloidosis, pulmonary alveolar proteinosis, seminal vesicle amyloid, cutaneous lichen amyloidosis, Mallory bodies, odontogenic (Pindborg) tumor amyloid, cancer, aging promoted by amyloid aggregation, or any disease caused by the misfolding or aggregation of proteins, or otherwise known by a person skilled in the art. The term “proteinopathy” may be used interchangeably with “proteopathy” as understood in the art.

As used herein, the term “amyloid” refers to fibrillar protein structures composed of stacked beta-sheet configurations. These fibrillar structures may be formed due to misfolding of proteins that have a normal or non-pathogenic structure, although there are also protein amyloids that are naturally occurring and/or non-pathogenic. Aggregation of various naturally occurring proteins have been associated with several pathogenic diseases. The accumulation of these fibrillar deposits can interfere with normal cellular structure and function, and can also induce additional protein molecules into aggregate forms. Histopathological identification of amyloid formation can be done with the use of dyes, such as Congo Red, which preferentially intercalate between the stacked beta-sheets. As used here, the term “amyloid aggregation” refers to the formation of these protein amyloids, such as in a cell and which may result in a pathogenic proteopathy.

As used herein, an “aggregation associated disease” refers to a disease that is associated with the aggregation of one or more naturally occurring proteins. Aggregation associated diseases include, but are not limited to, Alzheimer's disease (AD), cerebral amyloid angiopathy, frontotemporal lobar degeneration (FTLD), Pick's disease (PiD), progressive supranuclear palsy (PSP), corticobasal degeneration (CBD), global glial tauopathy (GGT), Lewy body disease (LBD), Parkinson's disease (PD), diffuse Lewy body disease (DLBD), Lewy body variant of Alzheimer's disease (LBV), synucleinopathies, multiple system atrophy (MSA), cardiovascular disease, atherosclerosis, coronary heart disease, stroke, TIA, peripheral arterial disease, aortic disease, brain disease, kidney disease, eye disease, high-intracellular cholesteryl ester/cholesterol accumulation, inflammation, Familial encephalopathy with neuroserpin inclusion bodies (FENIB), insulin-derived amyloidosis (LIDA), intracerebral hemorrhage (ICH), cognitive impairment, amyotrophic lateral sclerosis (ALS), Prion diseases, transmissible spongiform encephalopathy (TSE), Creutzfeldt-Jakob disease (CJD), fatal familial insomnia, Gerstmann-Straussler-Scheinker disease, myopathies, sarcopenia, idiopathic inflammatory myopathies (IIM), sporadic inclusion body myositis (sIBM), dermatomyositis (DM), polymyositis (PM), necrotizing autoimmune myopathy (NAM), transthyretin amyloidosis (ATTR), transthyretin amyloid cardiomyopathy (ATTR-CM), phenylketonuria (PKU), or human systemic amyloid disease.

Amyloid Beta (Aβ 40 and 42)—In AD, a the most common type of dementia, aggregation of Aβ peptides and formation of senile plaques is a central component and believed to occur early in the pathogenesis. Aβ peptides are cleaved from the amyloid precursor protein (APP) and aggregate into various forms, including oligomers, protofibrils and amyloid fibrils. Large and insoluble Aβ fibrils assemble into amyloid plaques, while Aβ oligomers are soluble and toxic to neurons. Cerebral amyloid angiopathy (CAA) is characterized by the deposition of Aβ in cerebral blood vessels and is believed to be a major contributor of cerebrovascular pathologies in AD.

Phospho Tau and tauopathies—Hyperphosphorylation of the microtubule-associated protein tau plays a key role in the pathogenesis of Alzheimer disease (AD) and other tauopathies, including corticobasal degeneration, post-encephalitic parkinsonism and argyrophilic grain disease. Tau hyperphosphorylation leads to loss of function, gain of toxicity and its aggregation, forming neurofibrillary tangles NFTs). Mutations in the gene cause familial forms of frontotemporal lobar degeneration (FTLD) including Pick's disease (PiD), progressive supranuclear palsy (PSP), corticobasal degeneration (CBD), and globular glial tauopathy (GGT).

Alpha-synuclein—Lewy body disease, multiple system atrophy: Alpha-synuclein is normally involved in trafficking of synaptic vesicles (SVs) in brain but its aggregation results in intraneuronal deposits called Lewy bodies (LBs) and extracellular Lewy neurites (LNs). Lewy bodies (LBs) is the pathological hallmark of Lewy body disease, which includes Parkinson's disease (PD), diffuse Lewy body disease (DLBD), and Lewy body variant of Alzheimer's disease (LBV). Lewy body dementia (LBD), which includes Dementia with Lewy bodies and Parkinson's disease dementia, is one of the most common types of dementia, after Alzheimer's disease. Most cases of synucleinopathies are sporadic however familial variants of alpha-synuclein leads to early onsets of PD. In multiple system atrophy (MSA), alpha-synuclein inclusions are mainly found in oligodendrocytes. See de Oliveira G A P, Silva J L. Alpha-synuclein stepwise aggregation reveals features of an early onset mutation in Parkinson's disease. Commun Biol. 2019 Oct. 11; 2:374; Schweighauser M, Shi Y, Tarutani A, Kametani F, Murzin A G, Ghetti B, Matsubara T, Tomita T, Ando T, Hasegawa K, Murayama S, Yoshida M, Hasegawa M, Scheres S H W, Goedert M. Structures of α-synuclein filaments from multiple system atrophy. Nature. 2020 September; 585(7825):464-469.

APOE2, E3, E4-AD: Apolipoprotein E (APOE) is an important lipid transporter with isoform-dependent effects on its lipidation and aggregation. APOE and its lipoprotein receptors in addition mediates Aβ transport. APOE4 allele, which is the strongest genetic risk factor for late onset Alzheimer's Disease, is less lipidated than APOE3 and APOE2, the latter being protective in Alzheimer's Disease. APOE4 appears to strongly affect oligomeric Amyloidβ aggregation and stabilization, thereby promoting Amyloidβ fibril formation in Alzheimer's Disease brains, and altering its lipidation state decreases Amyloids plaque burden. See Husain M A, Laurent B, Plourde M. APOE and Alzheimer's Disease: From Lipid Transport to Physiopathology and Therapeutics. Front Neurosci. 2021 Feb. 17; 15:630502; Parhizkar S, Holtzman D M. APOE mediated neuroinflammation and neurodegeneration in Alzheimer's disease. Semin Immunol. 2022 Feb. 26; 101594.

Cholesterol—atherosclerosis, cardiovascular disease and Alzheimer's disease: In blood, cholesterol is carried on two types of lipoproteins, of which low-density lipoprotein (LDL) contributes to cholesterol aggregation and build-up of atherosclerotic plaques in the arteries. Arteries then can become thick and stiff (arteriosclerosis) limiting blood flow, blood clots can form and arteries can even rupture. Atherosclerosis may lead to hypertension and cardiovascular disease, including coronary heart disease, strokes and TIAs, peripheral arterial disease, and aortic disease, and lead to damage in other organs including brain, kidneys and eyes. In AD brains, cholesterol in cell membranes, accumulated in so called lipid rafts and associated free cholesterol, acts as seed for Aβ aggregation and promotes formation of fibrils. See Abdullah S M, Defina L F, Leonard D, Barlow C E, Radford N B, Willis B L, Rohatgi A, McGuire D K, de Lemos J A, Grundy S M, Berry J D, Khera A. Long-Term Association of Low-Density Lipoprotein Cholesterol With Cardiovascular Mortality in Individuals at Low 10-Year Risk of Atherosclerotic Cardiovascular Disease Circulation. 2018 Nov. 20; 138(21):2315-2325; Habchi J, Chia S, Galvagnion C, Michaels T C T, Bellaiche M M J, Ruggeri F S, Sanguanini M, Idini I, Kumita J R, Sparr E, Linse S, Dobson C M, Knowles T P J, Vendruscolo M. Cholesterol catalyses Aβ42 aggregation through a heterogeneous nucleation pathway in the presence of lipid membranes. Nat Chem. 2018 June; 10(6):673-683; Hashemi M, Banerjee S, Lyubchenko Y L. Free Cholesterol Accelerates Aβ Self-Assembly on Membranes at Physiological Concentration. Int J Mol Sci. 2022 Mar. 3; 23(5):2803; Gellermann G P, Appel T R, Tannert A, Radestock A, Hortschansky P, Schroeckh V, Leisner C, Liitkepohl T, Shtrasburg S, Rocken C, Pras M, Linke R P, Diekmann S, Fandrich M. Raft lipids as common components of human extracellular amyloid fibrils. Proc Natl Acad Sci USA. 2005 May 3; 102(18):6297-302.

Cholesteryl (Co-Esteryl)—atherosclerosis, cardiovascular disease: Cholesteryl ester is the inactive and more hydrophobic form of cholesterol, in which cholesterol is esterified with fatty acids in order to be transported to target organs. It is therefore the major form of cholesterol in lipoproteins. At atherosclerotic plaques, aggregated LDL and cholesteryl esters are taken up by LDLr-related protein (LRP1) into vascular smooth muscle cells, endothelial cells and macrophages (rendering then foam macrophages), resulting in high-intracellular cholesteryl ester/cholesterol accumulation and inflammation. Llorente-Cortés V, Otero-Viñas M, Camino-López S, Costales P, Badimon L. Cholesteryl Esters of Aggregated LDL Are Internalized by Selective Uptake in Human Vascular Smooth Muscle Cells. Arterioscler Thromb Vasc Biol. 2006 January; 26(1):117-23.

Neuroserpin—FENIB: The serine protease inhibitor neuroserpin is an inhibitory serpin mainly expressed in brain, with physiological functions in synaptic development and plasticity. The structure of neuroserpin is essential for its function and leads to polymerization and the formation of inclusion bodies, the hallmark of serpinopathies. Familial encephalopathy with neuroserpin inclusion bodies (FENIB), is a rare genetic degenerative disorder affecting the brain and spinal cord, with clinical manifestations including dementia, myoclonic seizures and epilepsy. See D'Acunto E, Fra A, Visentin C, Manno M, Ricagno S, Galliciotti G, Miranda E. Neuroserpin: structure, function, physiology and pathology. Cell Mol Life Sci. 2021 October; 78(19-20):6409-6430.

Insulin—LIDA: With chronic administration of insulin, at the site of injection aggregation of insulin into insoluble fibrils can lead to localized insulin-derived amyloidosis (LIDA), a cutaneous lesion. See Ansari A M, Osmani L, Matsangos A E, Li Q K. Current insight in the localized insulin-derived amyloidosis (LIDA): clinico-pathological characteristics and differential diagnosis. Pathol Res Pract. 2017 October; 213(10):1237-1241; Das A, Shah M, Saraogi I. Molecular Aspects of Insulin Aggregation and Various Therapeutic Interventions. ACS Bio & Med Chem Au, 2022 January, 10.1021/acsbiomedchemau.1c00054.

Cystatin C—CAA, possibly ALS: Cystatin C is a cysteine protease inhibitor that controls lysosomal activities and extracellular proteases, and with aggregation, Cystatin C seem to lose its function. It is found to be aggregated and is deposited on the vessel walls along with Aβ peptide in cerebral amyloid angiopathy (CAA), with main clinical manifestations being intracerebral hemorrhage (ICH) and cognitive impairment. A mutation in Cystatin C increases its aggregation properties in inherited CAA. Cystatin C is present in Bunina bodies, the inclusion bodies found in the motor neurons of amyotrophic lateral sclerosis (ALS) spinal cords. See Sheikh A M, Wada Y, Tabassum S, Inagaki S, Mitaki S, Yano S, Nagai A. Aggregation of Cystatin C Changes Its Inhibitory Functions on Protease Activities and Amyloid β Fibril Formation. Int J Mol Sci. 2021 Sep. 7; 22(18):9682; March M E, Gutierrez-Uzquiza A, Snorradottir A O, Matsuoka L S, Balvis N F, Gestsson T, Nguyen K, Sleiman P M A, Kao C, Isaksson H J, Bragason B T, Olafsson E, Palsdottir A, Hakonarson H. NAC blocks Cystatin C amyloid complex aggregation in a cell system and in skin of HCCAA patients. Nat Commun. 2021 Mar. 23; 12(1):1827; Wada Y, Nagai A, Sheikh A M, Onoda K, Terashima M, Shiota Y, Araki A, Yamaguchi S. Co-localization of cystatin C and prosaposin in cultured neurons and in anterior horn neurons with amyotrophic lateral sclerosis. J Neurol Sci. 2018 Jan. 15; 384:67-74.

Prion protein—prion diseases including CJD: Prion diseases, or transmissible spongiform encephalopathy (TSE), are caused by misfolding followed by aggregation and accumulation in neuronal cells of the prion protein, PrP, and eventually spongiform degeneration is seen. It is highly infectious in nature and most cases are sporadic but genetic forms consists of familial Creutzfeldt-Jakob disease (CJD), fatal familial insomnia, and Gerstmann-Straussler-Scheinker disease.

Myostatin—myopathies, sarcopenia and myositis: Myostatin negatively regulates muscle growth and has an impact on molecular regulators of atrophy and hypertrophy in different myopathies and sarcopenia. Myostatin is upregulated in idiopathic inflammatory myopathies (IIM). IIM, or myositis, are autoimmune diseases characterized by muscle weakness and includes 5 subtypes. In sporadic inclusion body myositis (sIBM), myostatin aggregates and accumulates with A3, and secretion of misfolded myostatin is impaired.

Transthyretin—transthyretin amyloidosis, heart and kidney diseases and preeclampsia: The tetrameric thyroxine transport protein transthyretin (TTR) forms soluble oligomers and amyloid aggregates when dissociated into monomers. The aggregation of TTR is the cause of transthyretin amyloidosis (ATTR), a systemic amyloidosis, that can lead to heart and kidney diseases and preeclampsia. Transthyretin amyloid cardiomyopathy (ATTR-CM) is a cause of heart failure.

Phenylalanine—Phenylketonuria: Phenylketonuria (PKU) is an inherited metabolic disease characterized by abnormally high concentrations of the essential amino acid L-phenylalanine in blood and brain and that can lead to chronic kidney disease. The multitude of health problems associated with PKU includes disorders associated with it including anemia, rickets, atopic dermatitis, coronary heart disease, diabetes mellitus and arthritis. Formation of phenylalanine fibrils can initiate aggregation of proteins under physiological conditions, and the resultant fibrils can cause severe hemolysis.

NFL—motor neuron degeneration: Mutant neurofilament (NF) proteins are characterized by defective transport or assembly and NF aggregation or accumulation, leading to atrophy and motor neuron degeneration. Mutations in neurofilament light (NFL) subunit cause Charcot-Marie-Tooth disease, the most common inherited peripheral neuropathy, and NF mutations have been found in patients with early-onset PD, AD and sporadic Amyotrophic Lateral Sclerosis (ALS). In ALS, aggregation of NFL may also promote aggregation of wildly expressed proteins that are destabilized by missense mutations.

Fibrin—Cerebrovascular damage, AD, CAA: Iron-induced free radicals can generate thrombolysis-resistant fibrin-like polymers. These fibrin fibers can irreversibly trap red blood cells (RBCs) and in this way induce chronic hypoxia in the brain and cause other cerebrovascular damage. Insoluble deposits of fibrin and Aβ aggregates are present in AD brains and neurovasculature, leading to CAA and blood clots.

Lysozyme—human systemic amyloid disease: Lysozyme, like many other well-folded globular proteins, under stressful conditions produces nanoscale oligomer assembly and amyloid-like fibrillar aggregates. With engaging Raman microscopy, we made a critical structural analysis of oligomer and other assembly structures of lysozyme obtained from hen egg white and provided a quantitative estimation of a protein secondary structure in different states of its fibrillation. The accumulation in vital organs of amyloid fibrils made of mutational variants of lysozyme (HuL) is associated with a human systemic amyloid disease.

Complement protein C3 and C9 aggregation: The complement cascade is a critical effector mechanism of the innate immune system that contributes to the rapid clearance of pathogens and dead or dying cells, as well as contributing to the extent and limit of the inflammatory immune response. It has been demonstrate the ubiquitous, spatial and specific enrichment of C9 in amyloid deposits irrespective of amyloid-, organ- or tissue type. Our findings lend support to the hypothesis that amyloidosis might activate the complement cascade, which could lead to the formation of the membrane attack complex and cell death.

Crystallins: Cataracts are a common protein misfolding disease of the ocular lens, which affects approximately 50% of the population over the age of 65. This disease results from accumulated damage to lens Crystallin proteins, which destabilizes their folds and causes them to aggregate, resulting in the blurring of vision. Currently, the only treatment for cataracts is invasive surgical extraction that is carried out in the advanced stages of the disease. As a result, there is much interest in understanding the cause of cataracts and the mechanism by which they form. Kate L. Moreau, Jonathan A. King. Protein Misfolding and Aggregation in Cataract Disease and Prospects for Prevention. Trends Mol Med. 2012 May; 18(5): 273-282.

Atria Natriuretic Peptide: Atrial Natriuretic Peptide (ANP)-containing amyloid is frequently found in the elderly heart. that b-ANP plays a crucial role in ANP amyloid deposition under physio pathological congestive heart failure (CHF) conditions. It is also indicated that early isolated atrial amyloidosis (IAA)-related ANP deposition may occur in CHF and suggest that these latter patients should be monitored for the development of cardiac amyloidosis. Millucci L, Paccagnini E, Ghezzi L, Bernardini G, Braconi D, Laschi M, et al. (2011) Different Factors Affecting Human ANP Amyloid Aggregation and Their Implications in Congestive Heart Failure. PLoS ONE 6(7): e21870.

Calcitonin: Calcitonin is a hormone made by thyroid, a small, butterfly-shaped gland located near the throat. Calcitonin helps control how the body uses calcium. Calcitonin is a type of tumor marker. Tumor markers are substances made by cancer cells or by normal cells in response to cancer in the body. Calcitonin aggregation is Association with medullary carcinoma of the thyroid (MTC), and also limits its clinical application. Belfiore, M., Cariati, I., Matteucci, A. et al. Calcitonin native prefibrillar oligomers but not monomers induce membrane damage that triggers NMDA-mediated Ca2+-influx, LTP impairment and neurotoxicity. Sci Rep 9, 5144 (2019).

21 B-type natriuretic peptide (BNP): BNP and its N-terminal fragment (NT-proBNP) are released from ventricular cardiomyocytes in response to an increase in ventricular wall stress and to myocardial ischemia. Both BNP and NT-proBNP have proven to be reliable diagnostic and prognostic biomarkers in patients with heart failure. Weber M, Mitrovic V, Hamm C. B-type natriuretic peptide and N-terminal pro-B-type natriuretic peptide—Diagnostic role in stable coronary artery disease. Exp Clin Cardiol. 2006 Summer; 11(2):99-101.

Serum amyloid A (SAA): Serum amyloid A (SAA) protein is synthesized in the liver in normal conditions, but in AA amyloidosis, under the stimulus of AEF, SAA protein aggregates into fibrils and is deposited in the liver. Jayaraman S., Gantz D. L., Haupt C., Gursky O. Serum amyloid A forms stable oligomers that disrupt vesicles at lysosomal pH and contribute to the pathogenesis of reactive amyloidosis. Proc Natl Acad Sci USA. 2017; 114: E6507-E6515.

Islet amyloid Polypeptide (IAPP): Diabetes mellitus is a metabolic disease affecting an estimated 383 million people worldwide, of which about 90% suffer from T2D. T2D features an adult onset of the disease and its progression is characterized by pancreatic β-cell death, causing reduced insulin secretion. The disease mechanism of T2D is largely unknown and there is no known cure. Given the complex nature of the disease, the cause of 0-cell death is likely the result of interplay of many factors. For instance, since amyloid aggregates of IAPP (a.k.a. amylin) in pancreas are found in approximately 90% of patients upon postmortem examination, many research efforts focused on understanding IAPP aggregation and its association with the disease. Nedumpully-Govindan, P., Ding, F. Inhibition of IAPP aggregation by insulin depends on the insulin oligomeric state regulated by zinc ion concentration. Sci Rep 5, 8240 (2015).

As used herein, the term “technetium pyrophosphate (99mTc-PYP) scintigraphy” is a diagnostic method involving the use of a technetium radiotracer that bind to and can be used to localize accumulated amyloid deposits by gamma ray detection. One non-limiting example is the detection of cardiomyopathy caused by TTR amyloidosis. Whereas scintigraphy is two-dimensional, the same process can be used in 99mTc-PYP single-photon emission computed tomography (SPECT) for a three-dimensional scan.

The term “% w/w” or “% wt/wt” means a percentage expressed in terms of the weight of the ingredient or agent over the total weight of the composition multiplied by 100.

It is understood that an antibody with an antibody name described herein can be referred using a shortened version of the antibody name, as long as there are no conflicts with another antibody described herein. For example, F846C.1B2 can also be referred to as 846C.1B2, or 846.1B2. This can also refer to fragments of the antibody (e.g., with the same 1, 3, or 6 CDRs).

### Exemplary Methods of Use

Any of the anti-Gal3 antibodies or binding fragments thereof, or proteins, disclosed herein may be used in methods as provided herein.

Some aspects of the present disclosure are directed towards a method of promoting amyloid aggregation and/or oligomerization of a protein, comprising contacting the protein with Gal3, wherein Gal3 promotes amyloid aggregation and/or oligomerization of the protein.

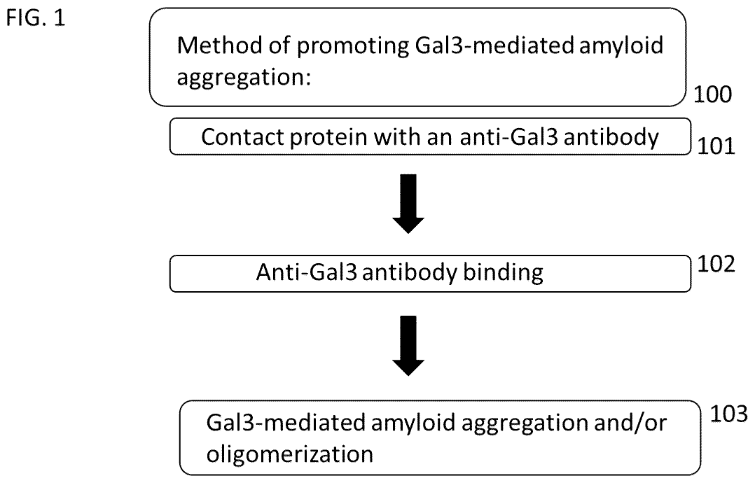

FIG. 1 is a flow chart depicting some embodiments of methods of promoting amyloid aggregation and/or oligomerization of a protein.

In some embodiments are disclosed methods of promoting amyloid aggregation and/or oligomerization of a protein 100. In some embodiments, the methods comprise contacting the protein with Gal3 101. In some embodiments, Gal3 binding 102 promotes amyloid aggregation and/or oligomerization of the protein 103. In some embodiments, the protein is contacted with Gal3 in an aqueous solution. In some embodiments, Gal3 promotes amyloid aggregation and/or oligomerization of the protein on the order of 0.5, 1, 2, 3, 4, 5, 6, 7, 8, 9, 10, 11, 12, 13, 14, 15, 16, 17, 18, 19, 20, 21, 22, 23, or 24 hours. In some embodiments, the protein comprises α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, or any combination thereof. In some embodiments, the tau protein is 4R tau and/or phosphorylated tau (phospho tau). In some embodiments, the phosphorylated tau is phospho-tau (S396). In some embodiments, the phosphorylated tau forms trimers, tetramers, or higher order oligomers when contacted with Gal3. In some embodiments, amyloid aggregation and/or oligomerization of the tau protein is achieved more rapidly compared to spontaneous aggregation and/or oligomerization of tau protein alone, or aggregation and/or oligomerization of tau protein when mixed with heparin and/or arachnoid acid. In some embodiments, amyloid aggregation and/or oligomerization of the tau protein is achieved more rapidly compared to aggregation and/or oligomerization of tau protein when mixed with heparin and/or arachnoid acid at 37° C. or about 37° C. In some embodiments, amyloid aggregation and/or oligomerization of the tau protein is achieved with no more than 0.5, 1, 2, 3, 4, 5, 6, 7, 8, 9, 10, 11, 12, 13, 14, 15, 16, 17, 18, 19, 20, 21, 22, 23, or 24 hours of contacting the tau protein with Gal3. This aggregation and/or oligomerization of tau protein by Gal3 occurs faster than previous methods, such as those involving the use of heparin and/or arachnoid acid. In some embodiments, the protein comprises APOE, prion protein, or NFL, or any combination thereof. In some embodiments, the APOE is APO-E4. In some embodiments, this positive formation of the aggregates or oligomers allows for a model for testing and/or confirming of molecules that can reverse and/or inhibit the formation of these oligomers/aggregates.

In some embodiments, the protein is contacted with Gal3 at a temperature of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any temperature within a range defined by any two of the aforementioned temperatures. In some embodiments, the protein is contacted with Gal3 at body temperature, 37° C., or about 37° C. In some embodiments, the protein is contacted with Gal3 below body temperature, below 37° C., or below about 37° C. In some embodiments, the protein is contacted with Gal3 at room temperature or about room temperature. In some embodiments, the protein is contacted with Gal3 at a temperature of about 18, 19, 20, 21, 22, 23, or 24° C., or any temperature within a range defined by any two of the aforementioned temperatures.

In some embodiments of the methods of promoting amyloid aggregation and/or oligomerization of a protein using Gal3, the protein and Gal3 are combined and incubated in an aqueous solution. In some embodiments, the protein is provided at 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 μg/mL, or any concentration within a range defined by any two of the aforementioned concentrations. In some embodiments, Gal3 is provided at 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 μg/mL, or any concentration within a range defined by any two of the aforementioned concentrations. In some embodiments, the protein and Gal3 are provided at the same, about the same, or similar concentrations, where similar concentrations may mean concentrations that are or are about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% within each other. In some embodiments, the protein and/or Gal3 is provided at 50, 60, 70, 80, 90, 100, 110, 120, 130, 140, or 150 μg/mL, or any concentration within a range defined by any two of the aforementioned concentrations. In some embodiments, the protein and/or Gal3 is provided at 100 μg/mL or about 100 μg/mL. In some embodiments, the aqueous solution is any solution that is compatible with proteins (e.g., any one or more of isotonic, mimicking biological conditions, buffered, minimizing protein denaturation or degradation, or maintaining proper protein folding). In some embodiments, the aqueous solution is saline or sodium phosphate buffer, optionally 10 mM sodium phosphate buffer. In some embodiments, the protein and Gal3 are first prepared in solutions that are compatible with proteins and mixed together to arrive at the aqueous solution. In some embodiments, the aqueous solution may be diluted, such as to adjust the concentration of components in the aqueous solution (e.g. buffers) or to adjust the concentration of the protein and/or Gal3. In some embodiments, the protein and Gal3 are combined and incubated for a period of time sufficient to promote amyloid aggregation and/or oligomerization of the protein. In some embodiments, the protein and Gal3 are combined and incubated for a period of time that is or is about 0, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, or 48 hours, or any period of time within a range defined by any two of the aforementioned periods of time. In some embodiments, the protein and Gal3 are combined and incubated at a temperature sufficient to promote amyloid aggregation and/or oligomerization of the protein. In some embodiments, the protein and Gal3 are combined and incubated at a temperature of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any temperature within a range defined by any two of the aforementioned temperatures. In some embodiments, the protein and Gal3 are combined and incubated at body temperature, 37° C. or about 37° C. In some embodiments, the protein and Gal3 are combined and incubated at below body temperature, below 37° C., or below about 37° C. In some embodiments, the protein and Gal3 are combined and incubated at room temperature or about room temperature. In some embodiments, the protein and Gal3 are combined and incubated at a temperature of about 18, 19, 20, 21, 22, 23, or 24° C., or any temperature within a range defined by any two of the aforementioned temperatures. The resultant protein, and amyloid aggregates and/or oligomers thereof may be assessed by methods generally known in the art, such as Western blot, dot blot, and ELISA.

In some embodiments, one can a) incubate Gal3 with IAPP (such as human IAPP) at 100 μg/mL concentration each in an Eppendorf tube under constant stirring, b) at regular intervals such as 0, 0.5, 1, 2, 3, 4, and 5 hours, an aliquot can be stored at −20° C. for oligomerization analysis using Western blot or dot blot, and c) one can blot with ill antibody, which specifically recognizes oligomer conformation, to determine the IAPP oligomerization over time in the presence of Gal3. In some embodiments, the protein can be substituted for α-synuclein, tau protein, TAR DNA-binding protein 43 (TDP-43), transthyretin (TTR), uromodulin, serum amyloid A (SAA), p53, or any other protein disclosed herein. In some embodiments, the ill antibody can be substituted for A11 antibody, which also binds to protein oligomers.

Some aspects of the present disclosure are directed towards a method of inhibiting Gal3-mediated amyloid aggregation of a protein, comprising: contacting the protein with an anti-Gal3 antibody or binding fragment thereof, wherein binding of the anti-Gal3 antibody or binding fragment thereof to Gal3 inhibits Gal3-mediated amyloid aggregation of the protein.

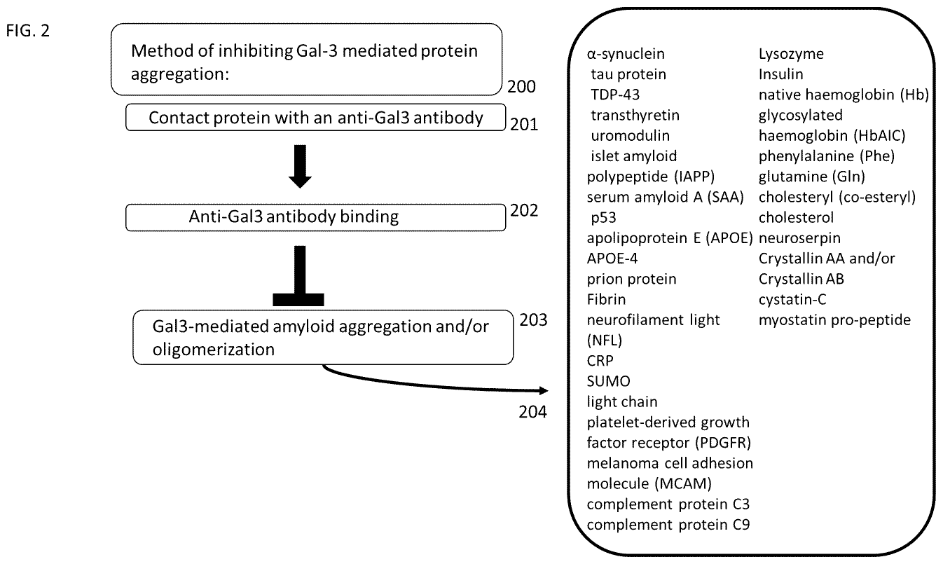

FIG. 2 is a flow chart depicting some embodiments of methods for inhibiting Gal3-mediated aggregation of a protein.

In some embodiments are disclosed methods of inhibiting Gal3-mediated amyloid aggregation of a protein 200. In some embodiments, the methods comprise contacting the protein with an anti-Gal3 antibody or binding fragment thereof 201. In some embodiments, binding of the anti-Gal3 antibody 202 or binding fragment thereof to Gal3 inhibits Gal3-mediated amyloid aggregation of the protein 203. In some embodiments, the protein is in a cell. In some embodiments, the protein 204 comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein.

Also disclosed herein are methods of inhibiting Gal3-mediated amyloid aggregation of a protein in a cell. In some embodiments, the methods comprise contacting the cell with an anti-Gal3 antibody or binding fragment thereof. In some embodiments, binding of the anti-Gal3 antibody or binding fragment thereof to Gal3 in the cell inhibits Gal3-mediated amyloid aggregation of the protein.

In some embodiments, the method is performed in vitro or in vivo.

In some embodiments, Gal3-mediated amyloid aggregation of the protein is inhibited by at least 5%, 10%, 15%, 20%, 25%, 30%, 35%, 40%, 45%, 50%, 55%, 60%, 65%, 70%, 75%, 80%, 85%, 90%, 95%, or 99%, or any percentage within a range defined by any two of the aforementioned percentages, after contacting with the anti-Gal3 antibody or binding fragment thereof relative to a cell that is not contacted with the anti-Gal3 antibody or binding fragment thereof.

In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein. In some embodiments, the phosphorylated tau forms trimers, tetramers, or higher order oligomers when contacted with Gal3.

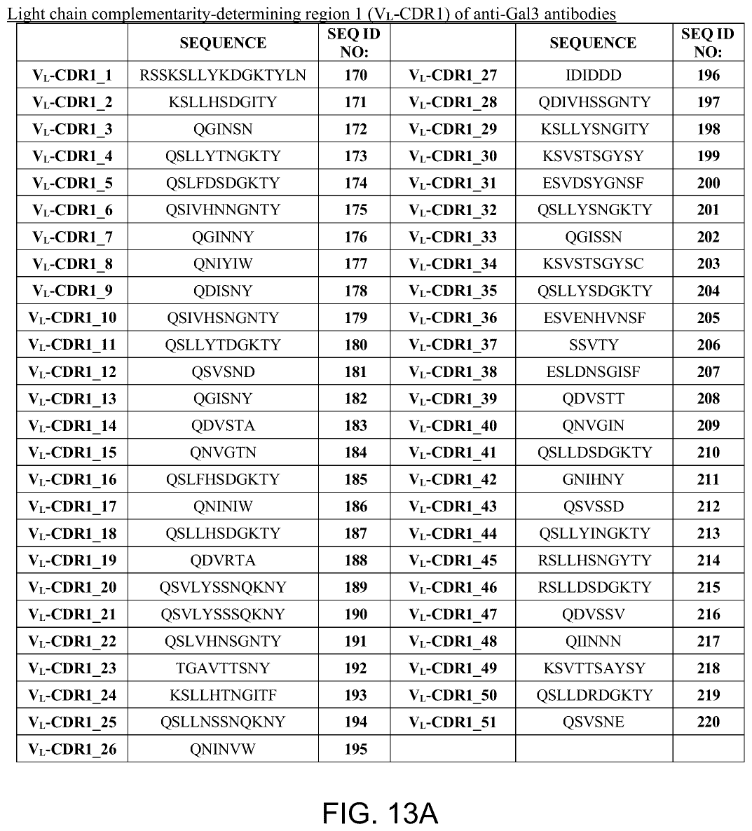

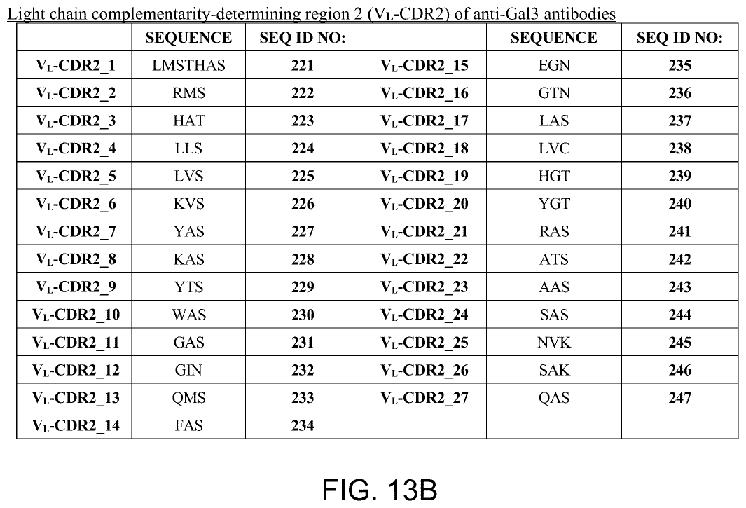

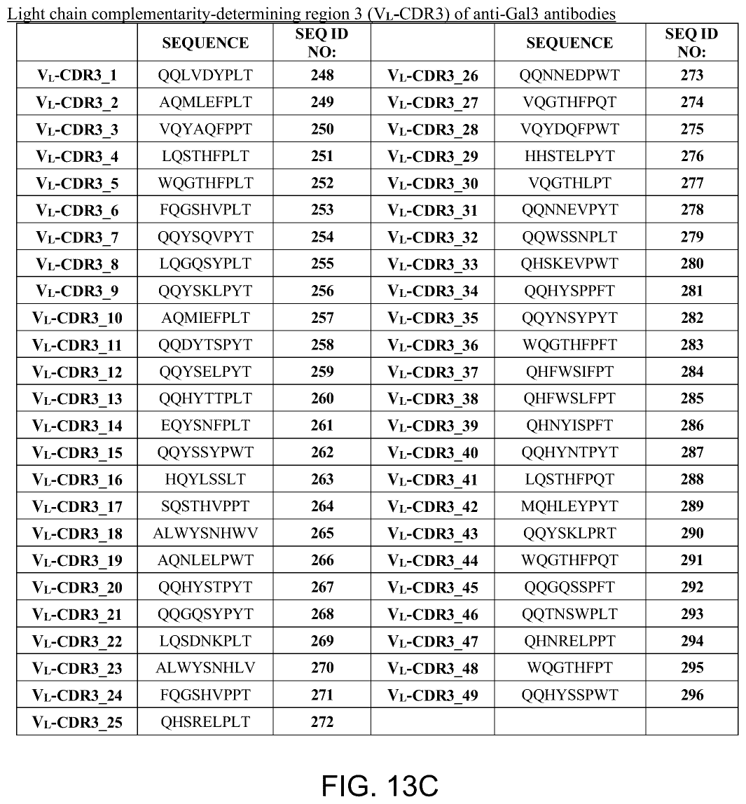

In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 211-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 13. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or a binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

Some aspects of the present disclosure are directed towards a method of treating an amyloid proteopathy in a subject in need thereof, comprising: administering to the subject an anti-Gal3 antibody or binding fragment thereof, wherein binding of the anti-Gal3 antibody or binding fragment thereof to Gal3 in the subject inhibits Gal3-mediated amyloid aggregation of a protein in the subject, thereby treating the amyloid proteopathy in the subject.

Some aspects of the present disclosure are directed towards a method of treating a proteopathy in a subject in need thereof, comprising: administering to the subject an anti-Gal3 antibody or binding fragment thereof, wherein binding of the anti-Gal3 antibody or binding fragment thereof to Gal3 in the subject inhibits Gal3-mediated oligomerization of a protein in the subject, thereby treating the proteopathy in the subject.

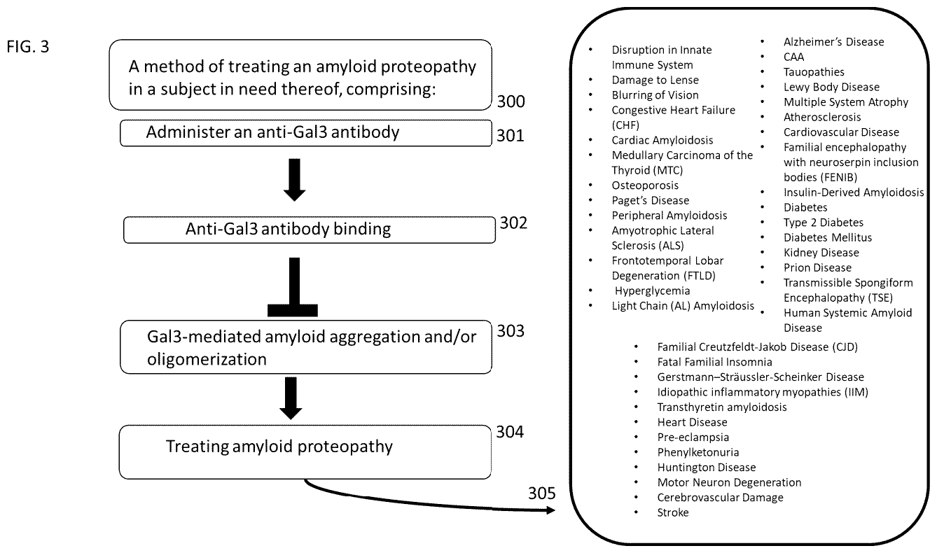

FIG. 3 is a flow chart depicting some embodiments of methods of treating an amyloid proteopathy.

In some embodiments are disclosed methods of treating an amyloid proteopathy in a subject in need thereof 300. In some embodiments are disclosed methods of treating a proteopathy in a subject in need thereof. In some embodiments, the methods comprise administering to the subject an anti-Gal3 antibody or binding fragment thereof 301. In some embodiments, binding of the anti-Gal3 antibody or binding fragment thereof to Gal3 302 in the subject inhibits Gal3-mediated amyloid aggregation of a protein 303 in the subject. In some embodiments, binding of the anti-Gal3 antibody or binding fragment thereof to Gal3 in the subject inhibits Gal3-mediated oligomerization of a protein in the subject. In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein. In some embodiments, inhibition of amyloid aggregation and/or oligomerization results in treatment of the amyloid proteopathy in the subject 304. In some embodiments, the proteopathy or amyloid proteopathy comprises familial Creutzfeldt-Jakob disease (CJD), Alzheimer's disease, CAA, tauopathies, Lewy body disease, multiple system atrophy, atherosclerosis, cardiovascular disease, familial encephalopathy with neuroserpin inclusion bodies (FENIB), insulin-derived amyloidosis, diabetes, type 2 diabetes, diabetes mellitus, kidney disease, prion disease, transmissible spongiform encephalopathy (TSE), human systemic amyloid disease, fatal familial insomnia, Gerstmann-Straussler-Scheinker disease, idiopathic inflammatory myopathies (IIM), transthyretin amyloidosis, heart disease, pre-eclampsia, phenylketonuria, Huntington disease, motor neuron degeneration, cerebrovascular damage, stroke disruption in innate immune system, damage to lenses, blurring of vision, congestive heart failure (CHF), cardiac amyloidosis, medullary carcinoma of the thyroid (MTC), osteoporosis, Paget's disease, peripheral amyloidosis, amyotrophic lateral sclerosis (ALS), frontotemporal lobar degeneration (FTLD), hyperglycemia, light chain amyloidosis (AL), a synucleinopathy, Parkinson's disease, dementia with Lewy bodies, progressive supranuclear palsy, corticobasal degeneration, Pick's disease, TDP-43 proteopathy, TTR amyloidosis (ATTR), uromodulin-associated kidney disease, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation, or any combination thereof.

In some embodiments, the protein is in a cell. In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein.

In some embodiments, the anti-Gal3 antibody, or binding fragment thereof, comprises TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, and/or 21H6-H6L4. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 blocking antibody blocks anti-Gal3 antibody binding to Gal3 by about 50%, 60%, 70% 75%, 80%, 81%, 82%, 83%, 84%, 85%, 86%, 87%, 88%, 89%, 90%, 91%, 92%, 93%, 94%, 95%, 96%, 97%, 98%, or 99% as compared to anti-Gal3 antibody binding in the absence of the anti-Gal3 blocking antibody, or blocks anti-Gal3 antibody binding to Gal3 by a range that is defined by any two of the preceding values. For example, in some embodiments, the anti-Gal3 blocking antibody blocks anti-Gal3 antibody binding by between about 50% and 100%, 50% and 95%, 50% and 90%, 50% and 85%, 50% and 80%, 50% and 75%, 50% and 70%, 50% and 60%, 60% and 100%, 60% and 95%, 60% and 90%, 60% and 85%, 60% and 80%, 60% and 75%, 75% and 100%, 75% and 95%, 75% and 90%, or 75% and 85%, as compared to anti-Gal3 antibody binding in the absence of the anti-Gal3 blocking antibody.

In some embodiments, the anti-Gal3 blocking antibody binds to one or more of the same epitopes as the anti-Gal3 antibody. In some embodiments, the antibody is one that competes for binding to any one or more of the proceeding antibodies at a level of at least 80% competition, e.g., Example 54.

In some embodiments, the anti-Gal3 antibody inhibits Gal3-mediated amyloid aggregation with at least 50%, 60%, 70%, 80%, 90%, 100% efficiency. For example, in some embodiments, binding of the anti-Gal3 antibody inhibits Gal3-mediated amyloid aggregation with at least 50%-100%, 50%-90%, 50%-80%, 50%-70%, or 70%-100% efficiency. In some embodiments, the anti-Gal3 antibody inhibits Gal3-mediated amyloid aggregation by at least 1-fold, 2-fold, 3-fold, 4-fold, 5-fold, 6-fold, 7-fold, 8-fold, 9-fold, or 10-fold, or by a range that is defined by any two of the preceding values. For example, in some embodiments, binding of the anti-Gal3 antibody inhibits Gal3-mediated amyloid aggregation by at least 1-fold to 10-fold, 1-fold to 7-fold, 1-fold to 5-fold, 1 fold-3 fold, 3-fold to 10-fold, 3-fold to 7-fold, 3-fold to 5-fold, 5-fold to 10-fold, or 5-fold to 7-fold.

In some embodiments, the methods further comprise identifying the subject as needing treatment of the amyloid proteopathy prior to the administering step.

In some embodiments, the methods further comprise detecting an improvement in the amyloid proteopathy in the subject following the administering step. In some embodiments, identifying the subject as needing treatment of the amyloid proteopathy and/or detecting the improvement in the amyloid proteopathy is done by biopsy, blood or urine test, echocardiogram, or technetium pyrophosphate (99mTc-PYP) scintigraphy.

In some embodiments, a method of inhibiting Gal3-mediated amyloid aggregation of amyloid β40 and/or amyloid p42 is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of amyloid β40 and/or amyloid β42.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of amyloid β40 and/or amyloid β42 in the subject, thereby treating Alzheimer's disease in the subject.

In some embodiments, a method of treating CAA in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of amyloid β40 and/or amyloid β42 in the subject, thereby treating CAA in the subject.

In some embodiments, a method of inhibiting Gal3-mediated amyloid aggregation of phospho tau is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of phospho tau.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of phospho tau in the subject, thereby treating Alzheimer's disease in the subject.

In some embodiments, a method of treating tauopathies in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of phospho tau in the subject, thereby treating the tauopathy in the subject.

In some embodiments, a method of inhibiting Gal3-mediated amyloid aggregation of alpha synuclein is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of alpha synuclein.

In some embodiments, a method of treating Lewy body disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of alpha synuclein in the subject, thereby treating Lewy body disease in the subject.

In some embodiments, a method of treating multiple system atrophy in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of alpha synuclein in the subject, thereby treating multiple system atrophy in the subject.

In some embodiments, a method of inhibiting Gal3-mediated amyloid aggregation of APOE-4 is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of APOE-4.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated oligomerization of APOE-4 in the subject, thereby treating Alzheimer's disease in the subject.

In some embodiments, a method of treating CAA in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of alpha synuclein in the subject, thereby treating CAA in the subject.

In some embodiments, a method of inhibiting Gal3-mediated amyloid aggregation of cholesterol is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of cholesterol.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cholesterol in the subject, thereby treating Alzheimer's disease in the subject.

In some embodiments, a method of treating cardiovascular disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cholesterol in the subject, thereby treating cardiovascular disease in the subject.

In some embodiments, a method of treating atherosclerosis disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cholesterol in the subject, thereby treating atherosclerosis in the subject.

In some embodiments, a method of inhibiting Gal3-mediated amyloid aggregation of cholesteryl is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of cholesteryl.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cholesteryl in the subject, thereby treating Alzheimer's disease in the subject.

In some embodiments, a method of treating cardiovascular disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cholesteryl in the subject, thereby treating cardiovascular disease in the subject.

In some embodiments, a method of treating atherosclerosis disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cholesteryl in the subject, thereby treating atherosclerosis in the subject.

In some embodiments, a method of inhibiting Gal3-mediated amyloid aggregation of neuroserpin is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of neuroserpin.

In some embodiments, a method of treating familial encephalopathy with neuroserpin inclusion bodies (FENIB) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of neuroserpin in the subject, thereby treating familial encephalopathy with neuroserpin inclusion bodies (FENIB) in the subject.

In some embodiments, a method of inhibiting Gal3-mediated amyloid aggregation of insulin is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of insulin.

In some embodiments, a method of treating insulin-derived amyloidosis in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of insulin in the subject, thereby treating insulin-derived amyloidosis in the subject.

In some embodiments, a method of treating diabetes in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of insulin in the subject, thereby treating diabetes in the subject.

In some embodiments, a method of inhibiting Gal3-mediated amyloid aggregation of cystatin-c is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of cystatin-c.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cystatin-c in the subject, thereby treating Alzheimer's disease in the subject.

In some embodiments, a method of treating CAA in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cystatin-c in the subject, thereby treating CAA in the subject.

In some embodiments, a method of treating kidney disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of cystatin-c in the subject, thereby treating kidney disease in the subject.

In some embodiments, a method of inhibiting Gal3-mediated amyloid aggregation of prion protein is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of prion protein.

In some embodiments, a method of treating prion disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of prion protein in the subject, thereby treating prion disease in the subject.

In some embodiments, a method of treating transmissible spongiform encephalopathy (TSE) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of prion protein in the subject, thereby treating transmissible spongiform encephalopathy (TSE) in the subject.

In some embodiments, a method of treating familial Creutzfeldt-Jakob disease (CJD) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of prion protein in the subject, thereby treating familial Creutzfeldt-Jakob disease (CJD) in the subject.

In some embodiments, a method of treating fatal familial insomnia in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of prion protein in the subject, thereby treating fatal familial insomnia in the subject.

In some embodiments, a method of treating Gerstmann-Straussler-Scheinker disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of prion protein in the subject, thereby treating Gerstmann-Straussler-Scheinker disease in the subject.

In some embodiments, a method of inhibiting Gal3-mediated amyloid aggregation of myostatin is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of myostatin.

In some embodiments, a method of treating idiopathic inflammatory myopathies (IIM) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of myostatin in the subject, thereby treating idiopathic inflammatory myopathies (IIM) in the subject.

In some embodiments, a method of inhibiting Gal3-mediated amyloid aggregation of transthyretin is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of transthyretin.

In some embodiments, a method of treating transthyretin amyloidosis in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of transthyretin in the subject, thereby treating transthyretin amyloidosis in the subject.

In some embodiments, a method of treating heart and/or kidney disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of transthyretin in the subject, thereby treating heart and/or kidney disease in the subject.

In some embodiments, a method of treating preeclampsia in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of transthyretin in the subject, thereby treating preeclampsia in the subject.

In some embodiments, a method of inhibiting Gal3-mediated amyloid aggregation of phenylalanine is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of phenylalanine.

In some embodiments, a method of treating phenylketonuria in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of phenylalanine in the subject, thereby treating phenylketonuria in the subject.

In some embodiments, a method of inhibiting Gal3-mediated amyloid aggregation of glutamine is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of glutamine.

In some embodiments, a method of treating Huntington Disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of glutamine in the subject, thereby treating Huntington Disease in the subject.

In some embodiments, a method of inhibiting Gal3-mediated amyloid aggregation of Neurofibrillary Light chain (NFL) is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of NFL.

In some embodiments, a method of treating motor neuron degeneration in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of NFL in the subject, thereby treating motor neuron degeneration in the subject.

In some embodiments, a method of inhibiting Gal3-mediated amyloid aggregation of fibrin is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of fibrin.

In some embodiments, a method of treating cerebrovascular damage in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of fibrin in the subject, thereby treating cerebrovascular damage in the subject.

In some embodiments, a method of treating stroke in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of fibrin in the subject, thereby treating stroke in the subject.

In some embodiments, a method of treating CAA in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of fibrin in the subject, thereby treating CAA in the subject.

In some embodiments, a method of treating Alzheimer's disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of fibrin in the subject, thereby treating Alzheimer's disease in the subject.

In some embodiments, a method of inhibiting Gal3-mediated amyloid aggregation of lysozyme is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of lysozyme.

In some embodiments, a method of treating human systemic amyloid disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of lysozyme in the subject, thereby treating human systemic amyloid disease in the subject.

In some embodiments, a method of inhibiting Gal3-mediated amyloid aggregation of complement proteins C3 and/or C9 is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of complement proteins C3 and/or C9.

In some embodiments, a method of treating disruption in innate immune system in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of complement proteins C3 and/or C9 in the subject, thereby treating disruption in innate immune system in the subject.

In some embodiments, a method of inhibiting Gal3-mediated amyloid aggregation of crystallins is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of crystallins.

In some embodiments, a method of treating damage to lenses and/or blurring of vision in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of crystallins in the subject, thereby treating damage to lenses and/or blurring of vision in the subject.

In some embodiments, a method of inhibiting Gal3-mediated amyloid aggregation of atrial natriuretic peptide (ANP) is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of ANP.

In some embodiments, a method of treating congestive heart failure (CHF) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of ANP in the subject, thereby treating CHF in the subject.

In some embodiments, a method of treating cardiac amyloidosis in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of ANP in the subject, thereby treating cardiac amyloidosis in the subject.

In some embodiments, a method of inhibiting Gal3-mediated amyloid aggregation of B-Type Natriuretic Peptide (BNP) is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of BNP.

In some embodiments, a method of treating congestive heart failure (CHF) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of BNP in the subject, thereby treating CHF in the subject.

In some embodiments, a method of treating cardiac amyloidosis in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of BNP in the subject, thereby treating cardiac amyloidosis in the subject.

In some embodiments, a method of inhibiting Gal3-mediated amyloid aggregation calcitonin is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of calcitonin.

In some embodiments, a method of treating medullary carcinoma of the thyroid (MTC) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of calcitonin in the subject, thereby treating MTC in the subject.

In some embodiments, a method of treating osteoporosis in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of calcitonin in the subject, thereby treating osteoporosis in the subject.

In some embodiments, a method of treating Paget's Disease in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of calcitonin in the subject, thereby treating Paget's Disease in the subject.

In some embodiments, a method of inhibiting Gal3-mediated amyloid aggregation Serum Amyloid (A) (SAA) is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of Serum Amyloid (A) (SAA).

In some embodiments, a method of treating peripheral amyloidosis in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of Serum Amyloid (A) (SAA) in the subject, thereby treating peripheral amyloidosis in the subject.

In some embodiments, a method of inhibiting Gal3-mediated amyloid aggregation of islet amyloid polypeptide (IAPP) is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of IAPP.

In some embodiments, a method of treating type 2 diabetes in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of IAPP in the subject, thereby treating type 2 diabetes in the subject.

In some embodiments, a method of inhibiting Gal3-mediated amyloid aggregation of TAR DNA binding protein 43 (TDP-43) is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated amyloid aggregation of TDP-43.

In some embodiments, a method of treating amyotrophic lateral sclerosis (ALS) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of TDP-43 in the subject, thereby treating ALS in the subject.

In some embodiments, a method of treating frontotemporal lobar degeneration (FTLD) in a subject in need thereof is disclosed. In some embodiments, the method comprises: administering to the subject an anti-Gal3 antibody or binding fragment thereof, wherein binding of the anti-Gal3 antibody or binding fragment thereof to Gal3 in the subject inhibits Gal3-mediated amyloid aggregation of TDP-43 in the subject, thereby treating FTLD in the subject.

In some embodiments, the amyloid proteopathy is reduced by at least 5%, 10%, 15%, 20%, 25%, 30%, 35%, 40%, 45%, 50%, 55%, 60%, 65%, 70%, 75%, 80%, 85%, 90%, 95%, or 99%, or any percentage within a range defined by any two of the aforementioned percentages, after the administering step relative to the amyloid proteopathy prior to the administering step. In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination thereof. In some embodiments, the tau protein is 4R tau, or phosphorylated tau (phospho tau). In some embodiments, the phosphorylated tau is phospho-tau (S396). In some embodiments, the phosphorylated tau forms trimers, tetramers, or higher order oligomers when contacted with Gal3. In some embodiments, the amyloid proteopathy comprises familial Creutzfeldt-Jakob disease (CJD), Alzheimer's disease, CAA, tauopathies, Lewy body disease, multiple system atrophy, atherosclerosis, cardiovascular disease, familial encephalopathy with neuroserpin inclusion bodies (FENIB), insulin-derived amyloidosis, diabetes, type 2 diabetes, diabetes mellitus, kidney disease, prion disease, transmissible spongiform encephalopathy (TSE), human systemic amyloid disease, fatal familial insomnia, Gerstmann-Straussler-Scheinker disease, idiopathic inflammatory myopathies (IIM), transthyretin amyloidosis, heart disease, pre-eclampsia, phenylketonuria, Huntington disease, motor neuron degeneration, cerebrovascular damage, stroke disruption in innate immune system, damage to lenses, blurring of vision, congestive heart failure (CHF), cardiac amyloidosis, medullary carcinoma of the thyroid (MTC), osteoporosis, Paget's disease, peripheral amyloidosis, amyotrophic lateral sclerosis (ALS), frontotemporal lobar degeneration (FTLD), hyperglycemia, light chain amyloidosis (AL), a synucleinopathy, Parkinson's disease, dementia with Lewy bodies, progressive supranuclear palsy, corticobasal degeneration, Pick's disease, TDP-43 proteopathy, TTR amyloidosis (ATTR), uromodulin-associated kidney disease, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation, or any combination thereof.

In some embodiments, the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 211-247. In some embodiments, the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 13. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 955-968, 1067-1109, 1415-1439. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

As applied to any of the methods of use disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to an epitope present within a region of Gal3 defined by Peptide 1 (ADNFSLHDALSGSGNPNPQG; SEQ ID NO: 3), Peptide 4 (GAGGYPGASYPGAYPGQAPP; SEQ ID NO: 6), Peptide 6 (GAYPGQAPPGAYPGAPGAYP; SEQ ID NO: 8), Peptide 7 (AYPGAPGAYPGAPAPGVYPG; SEQ ID NO: 9), or a combination thereof. In some embodiments, any of the methods disclosed herein involving an anti-Gal3 antibody or binding fragment can be performed with an antigen binding molecule that binds to Gal3. In some embodiments, any antibody (human, humanized, or not) that competes for binding to any one or more of the proceeding epitopes (optionally at, at least 80% competition as defined in the present examples, e.g., Example 54), can be used in the method as well. In some embodiments, the antibody binds to the same or overlapping epitope of any one or more of the preceding antibodies.

As applied to any of the methods of use disclosed herein, in some embodiments, the anti-Gal3 antibody or binding fragment thereof binds to an epitope of Gal3 that includes a motif of GxYPG, where x is the amino acids alanine (A), glycine (G), or valine (V). In some embodiments, an anti-Gal3 antibody as described herein binds to an epitope of Gal3 that includes two GxYPG motifs separated by three amino acids, where x is A, G, or V. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof comprises (1) a heavy chain variable region comprising a V_{H}-CDR1, a V_{H}-CDR2, and a V_{H}-CDR3 and (2) a light chain variable region comprising a V_{L}-CDR1, a V_{L}-CDR2, and a V_{L}-CDR3. In some embodiments, the V_{H}-CDR1 comprises an amino acid sequence having at least 60%, at least 70%, at least 80%, at least 90%, or 100% sequence identity to any amino acid sequence according to SEQ ID NOs: 27-70. In some embodiments, the V_{H}-CDR2 comprises an amino acid sequence having at least 60%, at least 70%, at least 80%, at least 90%, or 100% sequence identity to any amino acid sequence according to SEQ ID NOs: 71-111, 801, 951, 952. In some embodiments, the V_{H}-CDR3 comprises an amino acid sequence having at least 60%, at least 70%, at least 80%, at least 90%, or 100% sequence identity to any amino acid sequence according to SEQ ID NOs: 112-169, 802, 953, 954. In some embodiments, the V_{L}-CDR1 comprises an amino acid sequence having at least 60%, at least 70%, at least 80%, at least 90%, or 100% sequence identity to any amino acid sequence according to SEQ ID NOs: 170-220. In some embodiments, the V_{L}-CDR2 comprises an amino acid sequence having at least 60%, at least 70%, at least 80%, at least 90%, or 100% sequence identity to any amino acid sequence according to SEQ ID NOs: 211-247. In some embodiments, the V_{L}-CDR3 comprises an amino acid sequence having at least 60%, at least 70%, at least 80%, at least 90%, or 100% sequence identity to any amino acid sequence according to SEQ ID NOs: 248-296. In some embodiments, any of the methods disclosed herein involving an anti-Gal3 antibody or binding fragment can be performed with an antigen binding molecule that binds to Gal3. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

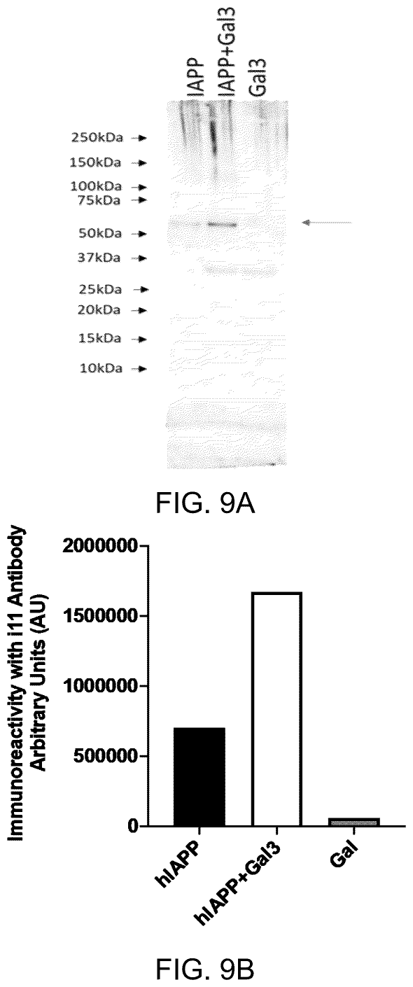

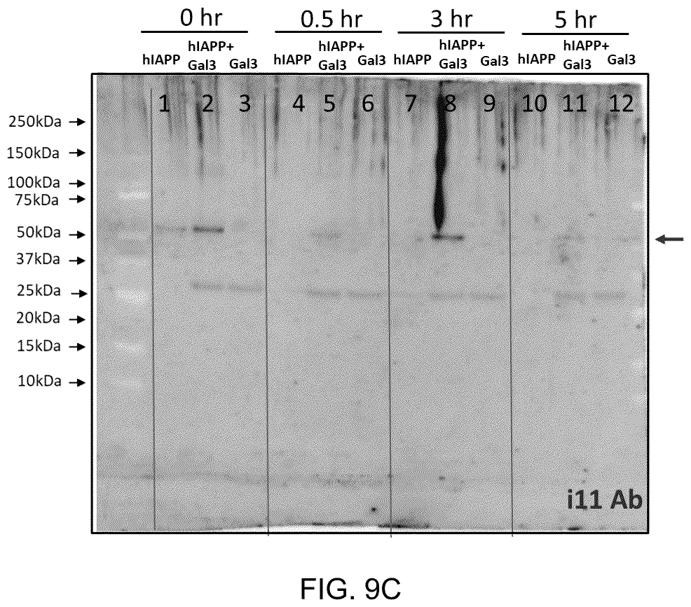

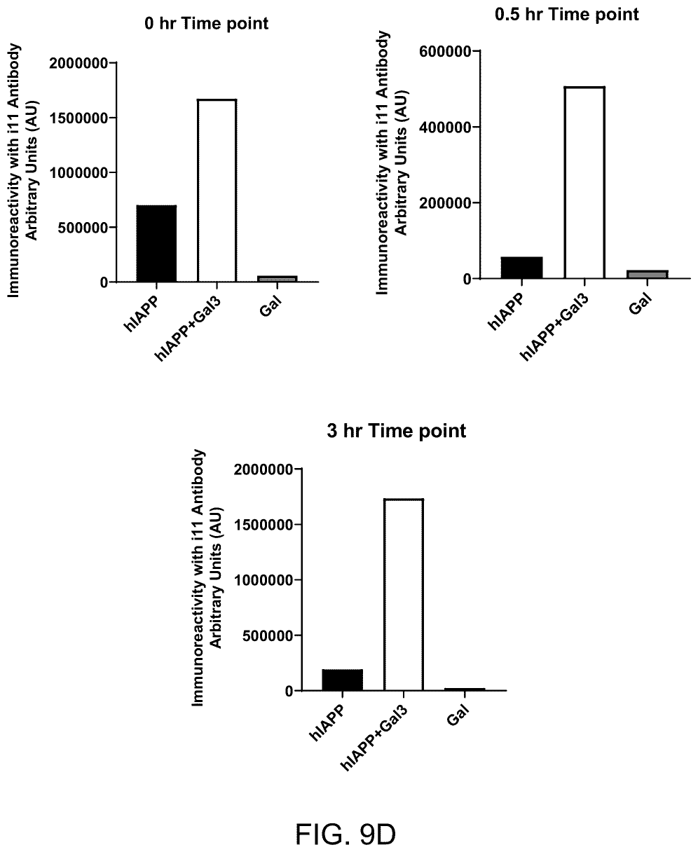

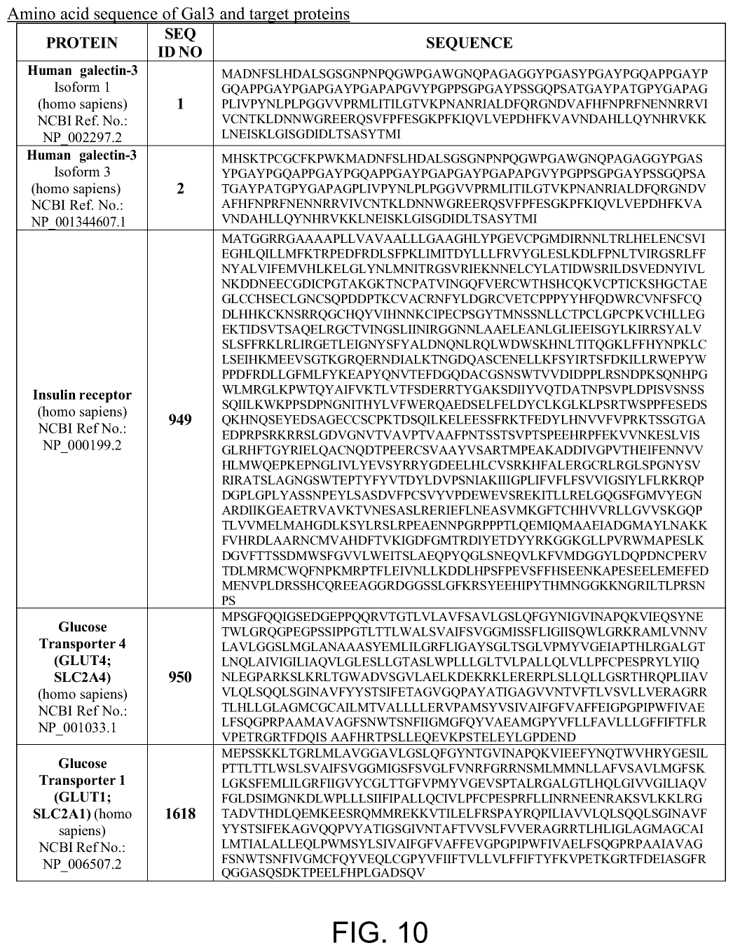

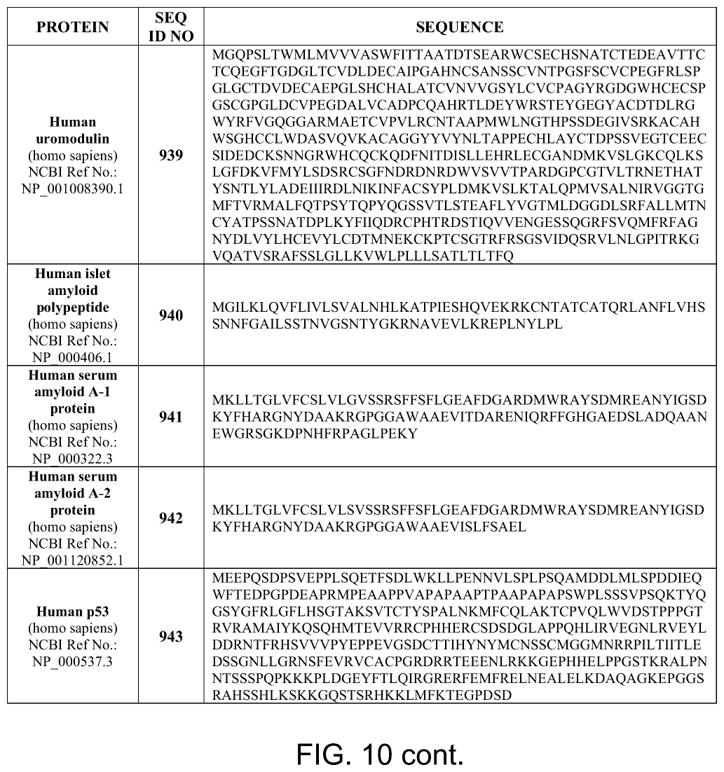

As applied to any of the methods of use disclosed herein, in some embodiments, exemplary V_{H}-CDR1 sequences are depicted in FIG. 9A. In some embodiments, exemplary V_{H}-CDR2 sequences are depicted in FIG. 9B. In some embodiments, exemplary V_{H}-CDR3 sequences are depicted in FIG. 9C. In some embodiments, exemplary V_{L}-CDR1 sequences are depicted in FIG. 10A. In some embodiments, exemplary V_{L}-CDR2 sequences are depicted in FIG. 10B. In some embodiments, exemplary V_{L}-CDR3 sequences are depicted in FIG. 10C.

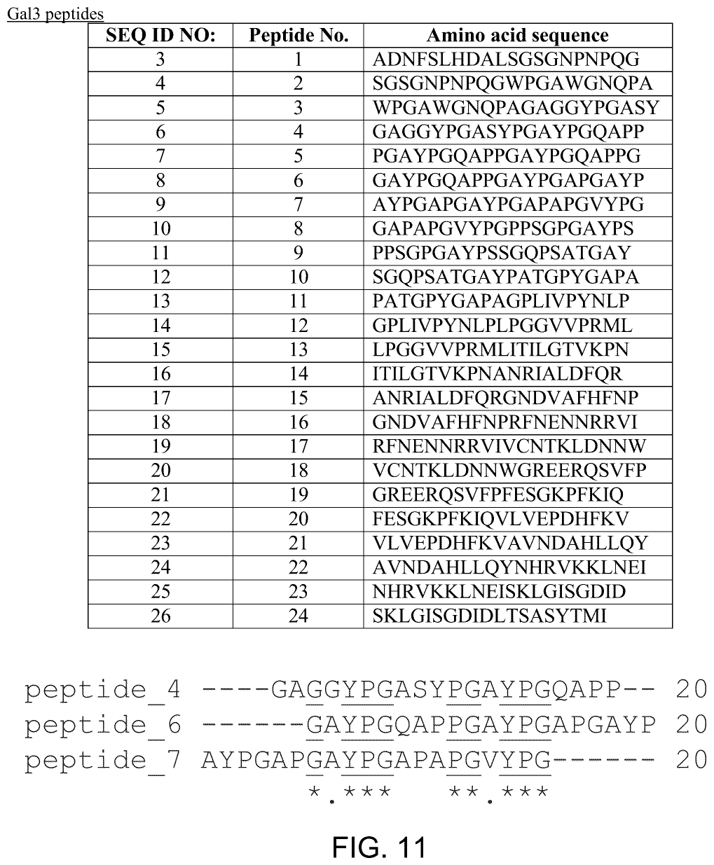

As applied to any of the methods of use disclosed herein, in some embodiments, the heavy chain variable region of any of the anti-Gal3 antibodies or binding fragments thereof disclosed herein comprises an amino acid sequence having at least 75%, 80%, 85%, 90%, 95%, or 100% sequence identity to any sequence according to SEQ ID NOs: 297-373, 803, 806-820, 955-968, 1067-1109, 1415-1439. In some embodiments, the heavy chain variable region of any of the anti-Gal3 antibodies or binding fragments thereof disclosed herein is selected from the group consisting of at least one of SEQ ID NOs: 297-373, 803, 806-820, 955-968, 1067-1109, 1415-1439. In some embodiments, exemplary V_{H} are depicted in FIG. 11. In some embodiments, any of the methods disclosed herein involving an anti-Gal3 antibody or binding fragment can be performed with an antigen binding molecule that binds to Gal3. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

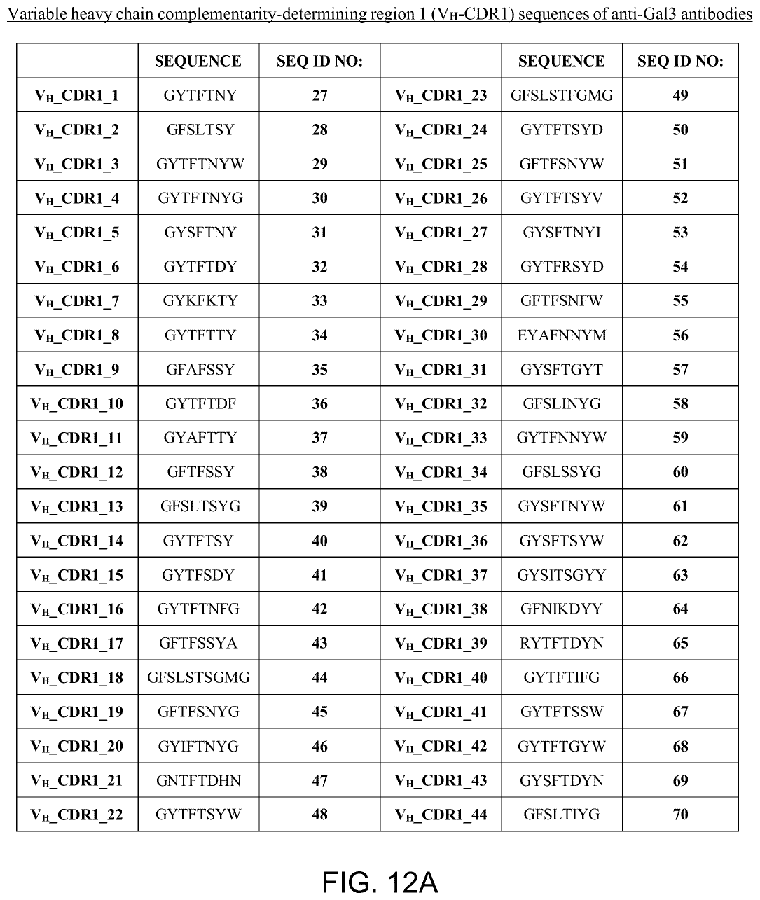

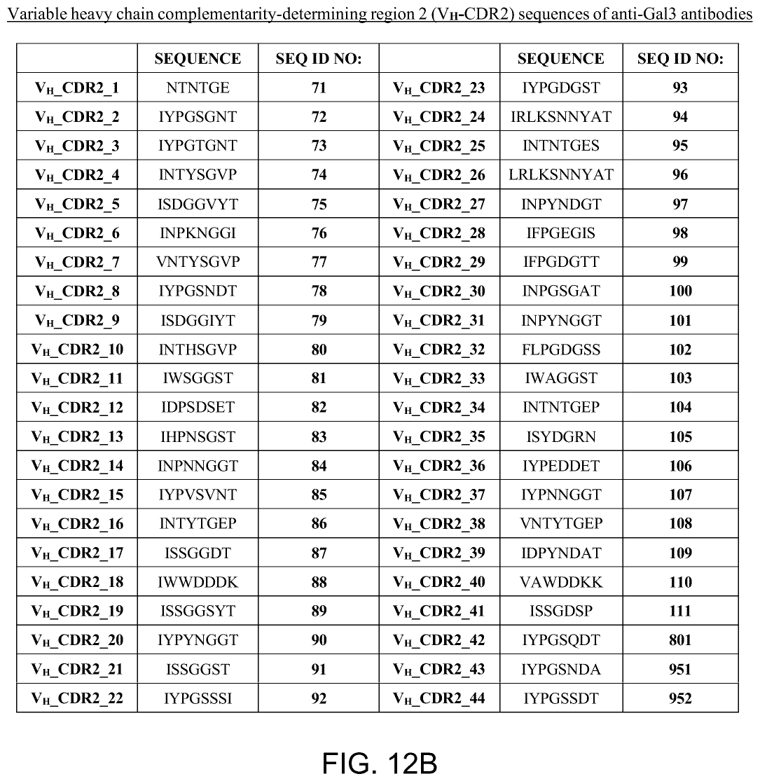

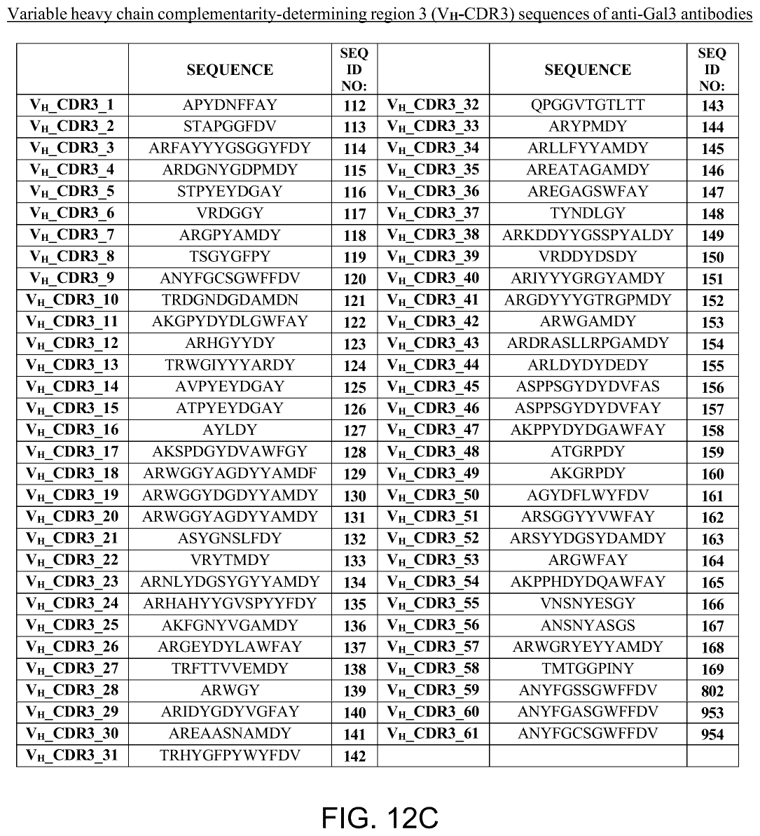

As applied to any of the methods of use disclosed herein, in some embodiments, the light chain variable region of any of the anti-Gal3 antibodies or binding fragments thereof disclosed herein comprises an amino acid sequence having at least 75%, 80%, 85%, 90%, 95%, or 100% sequence identity to any sequence according to SEQ ID NOs: 374-447, 821-835, 941-943, 969-982, 1110-1152, 1440-1464. In some embodiments, the light chain variable region of any of the anti-Gal3 antibodies or binding fragments thereof disclosed herein is selected from the group consisting of at least one of SEQ ID NOs: 374-447, 821-835, 941-943, 969-982, 1110-1152, 1440-1464. In some embodiments, exemplary V_{L} are depicted in FIG. 12. In some embodiments, any of the methods disclosed herein involving an anti-Gal3 antibody or binding In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies, or binding fragment thereof, can be performed with an antigen binding molecule that binds to Gal3.

As applied to any of the methods of use disclosed herein, in some embodiments, the anti-Gal3 antibody or binding fragment thereof comprises the heavy chain sequence of any one of SEQ ID NOs: 448-494, 804, 836-850, 983-996, 1153-1195, 1411, 1465-1489. In some embodiments, the anti-Gal3 antibody or binding fragment thereof comprises the light chain sequence of any one of SEQ ID NOs: 495-538, 805, 851-865, 997-1010, 1196-1238, 1412, 1490-1514. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

As applied to any of the methods of use disclosed herein, in some embodiments, the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

As applied to any of the methods of use disclosed herein, in some embodiments, the anti-Gal3 antibody or binding fragment thereof comprises a payload. In some embodiments, the payload is conjugated to the anti-Gal3 antibody or binding fragment thereof. In some embodiments, the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, or any combination thereof. In some embodiments, the payload is a detectable moiety. In some embodiments, any of the methods disclosed herein involving an anti-Gal3 antibody or binding fragment can be performed with an antigen binding molecule that binds to Gal3.

As applied to any of the methods of use disclosed herein, in some embodiments, the anti-Gal3 antibody or binding fragment thereof is or comprises a humanized antibody. In some embodiments, the anti-Gal3 antibody or binding fragment thereof is or comprises a full-length antibody or a binding fragment thereof. In some embodiments, the anti-Gal3 antibody or binding fragment thereof is or comprises a bispecific antibody or a binding fragment thereof. In some embodiments, the anti-Gal3-antibody or binding fragment thereof is or comprises a monovalent Fab′, a divalent Fab2, a single-chain variable fragment (scFv), a diabody, a minibody, a nanobody, a single-domain antibody (sdAb), or a camelid antibody, or binding fragment thereof. In some embodiments, the anti-Gal3 antibody or binding fragment thereof is or comprises an IgG framework. In some embodiments, the anti-Gal3 antibody or binding fragment thereof is or comprises an IgG1, IgG2, or IgG4 framework. In some embodiments, any of the methods disclosed herein involving an anti-Gal3 antibody or binding fragment can be performed with an antigen binding molecule that binds to Gal3.

As applied to any of the methods of treatment disclosed herein, in some embodiments, in some embodiments, the anti-Gal3 antibody or binding fragment thereof is administered enterally, orally, intranasally, parenterally, intracranially, subcutaneously, intramuscularly, intradermally, or intravenously, or any combination thereof.

As applied to any of the methods of treatment disclosed herein, in some embodiments, the anti-Gal3 antibody or binding fragment thereof is formulated for systemic administration. In some embodiments, the anti-Gal3 antibody or binding fragment thereof is formulated for parenteral administration. In some embodiments, more than one anti-Gal3 antibody or binding fragment is administered. In some embodiments, when more than one anti-Gal3 antibody or binding fragment is administered, the more than one anti-Gal3 antibodies or binding fragments thereof may be selected from the anti-Gal3 antibodies or binding fragments thereof disclosed herein. In some embodiments, any of the methods disclosed herein involving an anti-Gal3 antibody or binding fragment can be performed with an antigen binding molecule that binds to Gal3.

As applied to any of the methods of use or treatment disclosed herein, the subject is a mammal. In some embodiments, the mammal is a human, cat, dog, mouse, rat, hamster, rodent, pig, cow, horse, sheep, or goat. In some embodiments, the mammal is a human.

### Exemplary Anti-Gal3 Antibodies and Binding Fragments Thereof

Disclosed herein and as applicable to any of the methods or uses disclosed herein are anti-Gal3 antibodies or binding fragments thereof. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3. In some embodiments, any of the anti-Gal3 antibodies or binding fragments thereof or any arrangement of any of the anti-Gal3 antibodies or binding fragments provided herein may be substituted with an antigen binding molecule that binds to Gal3.

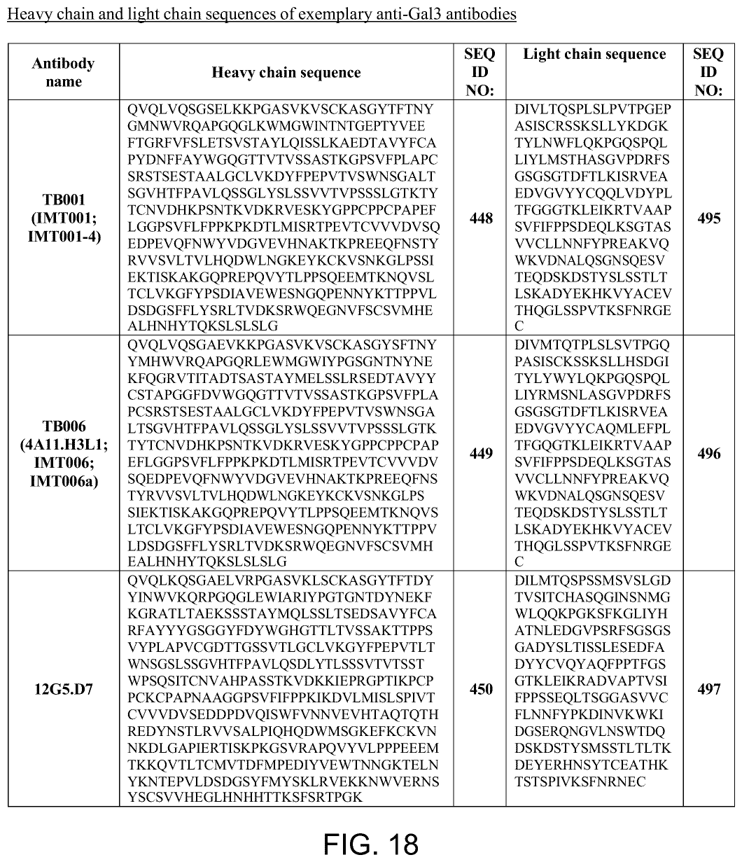

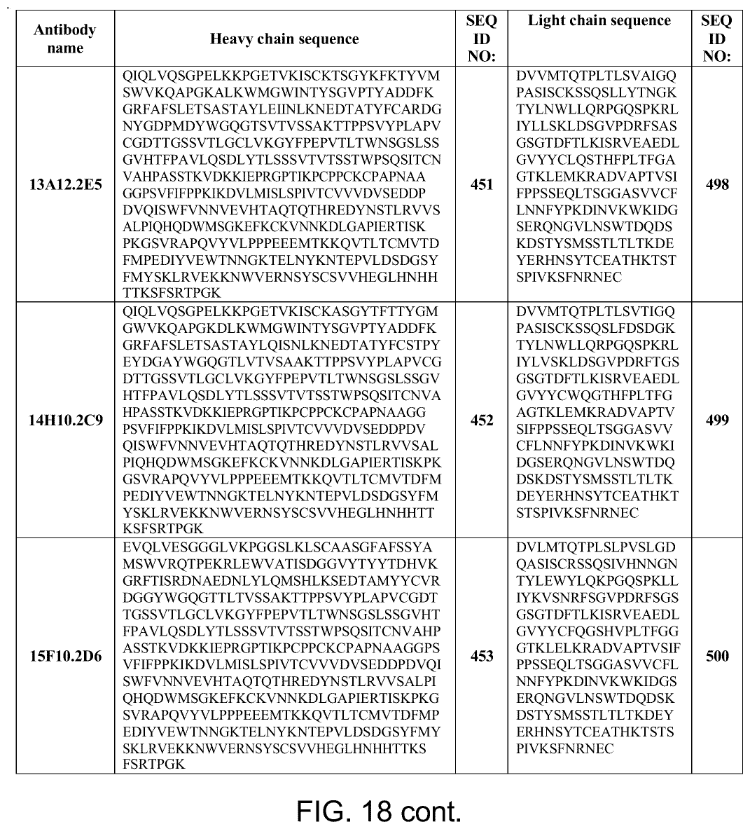

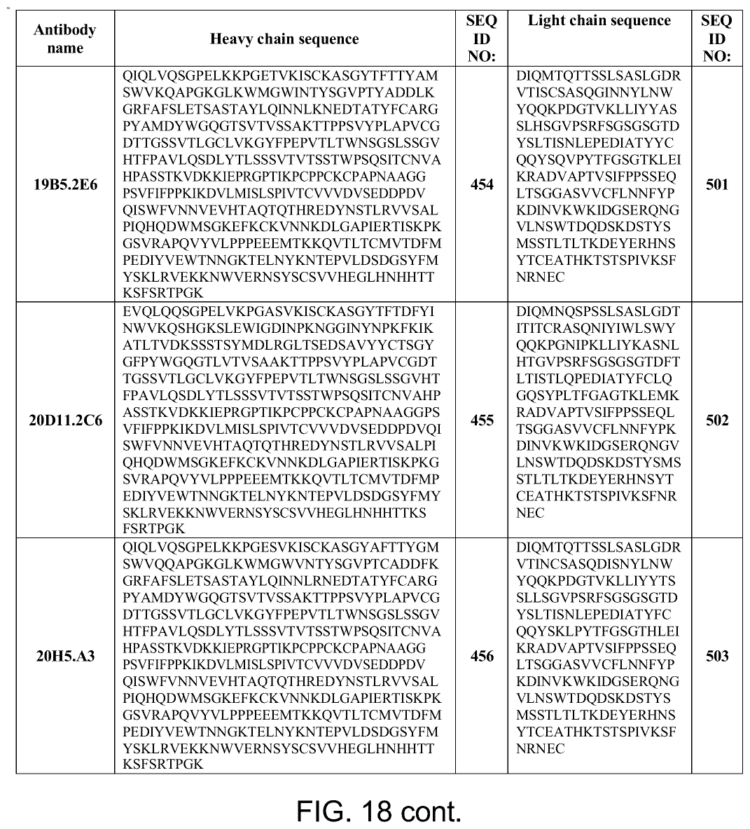

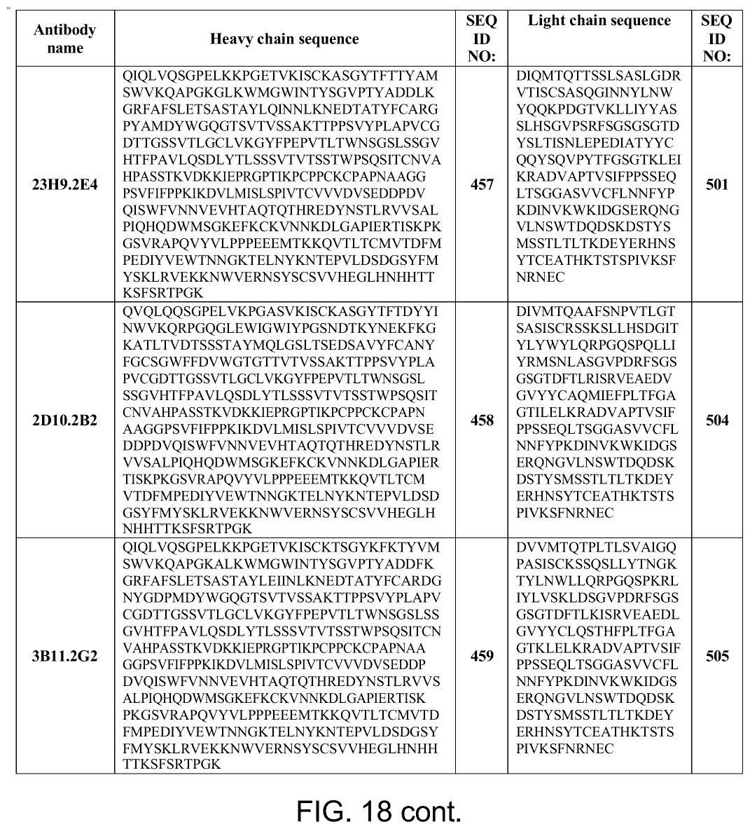

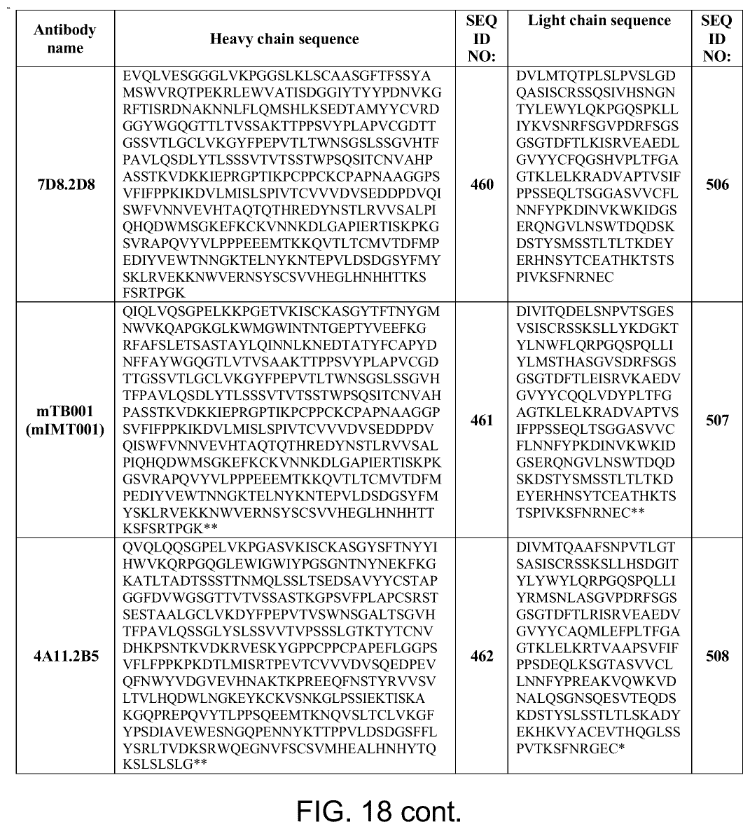

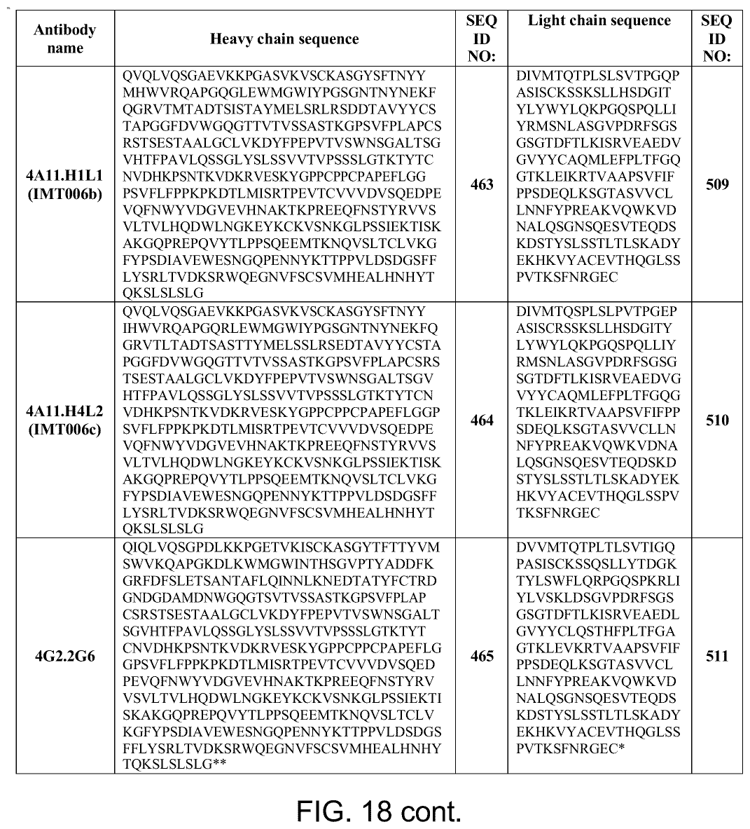

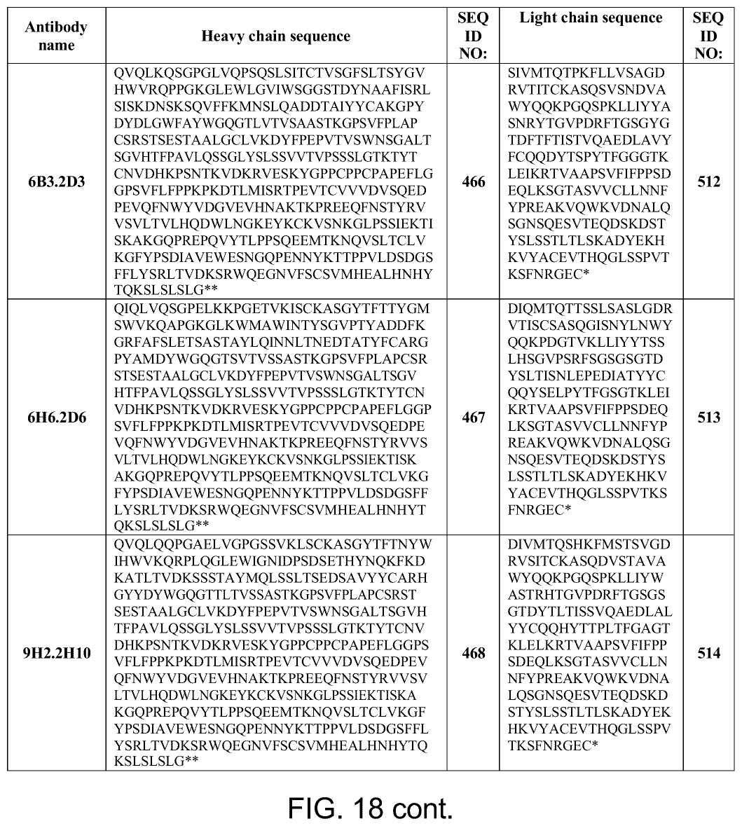

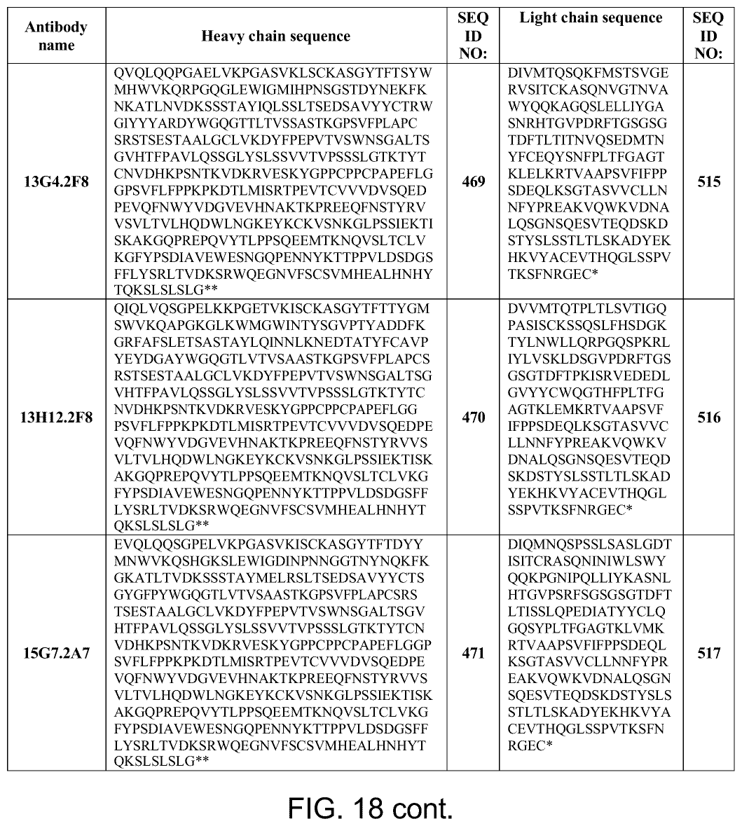

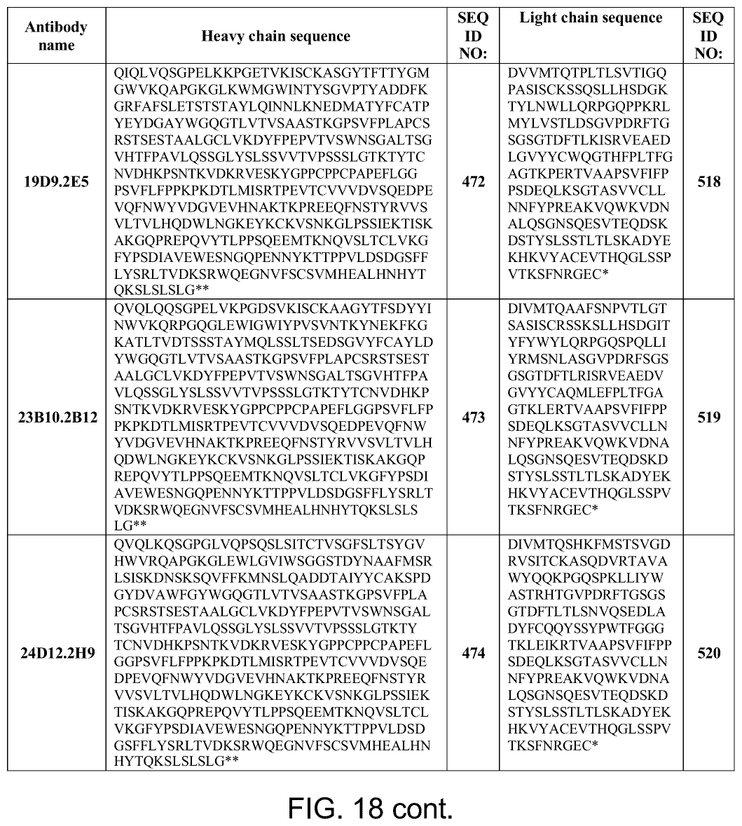

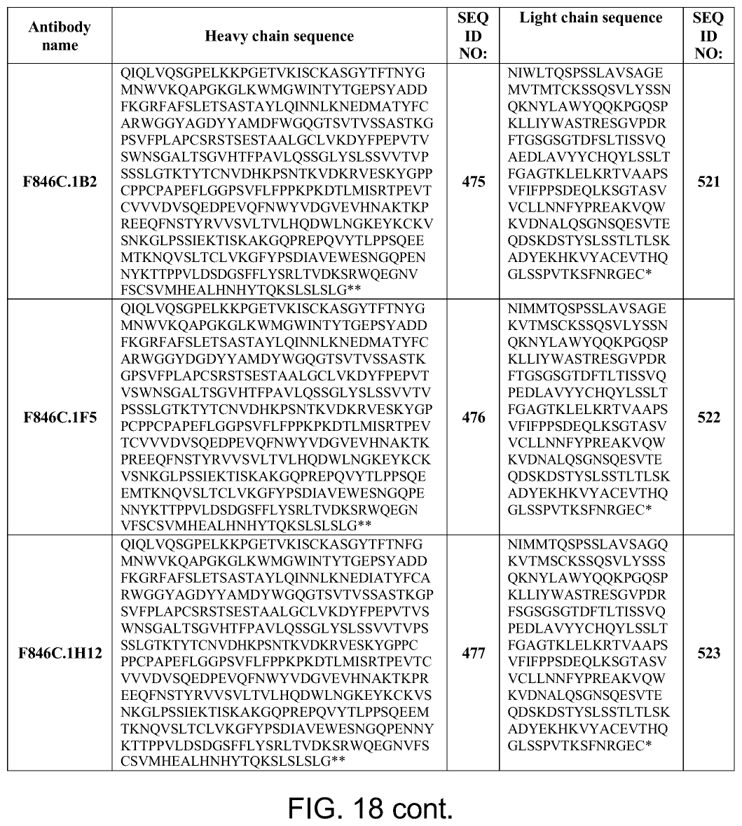

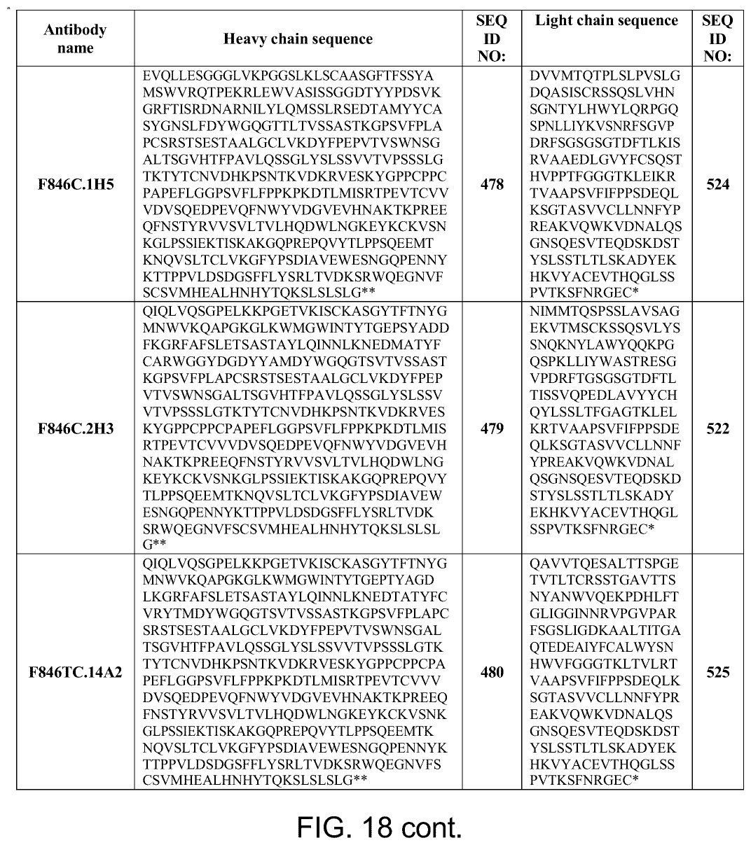

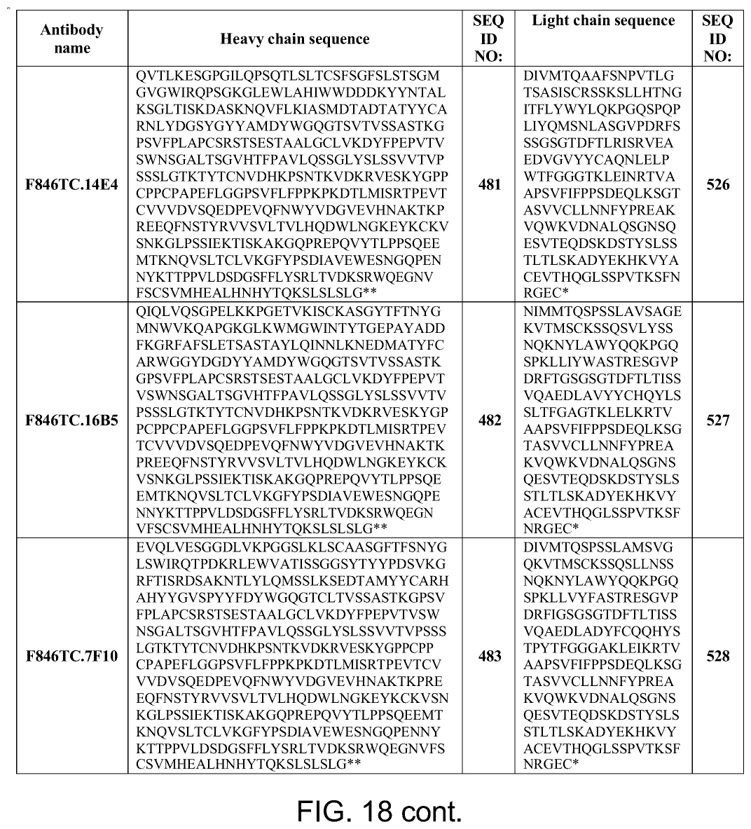

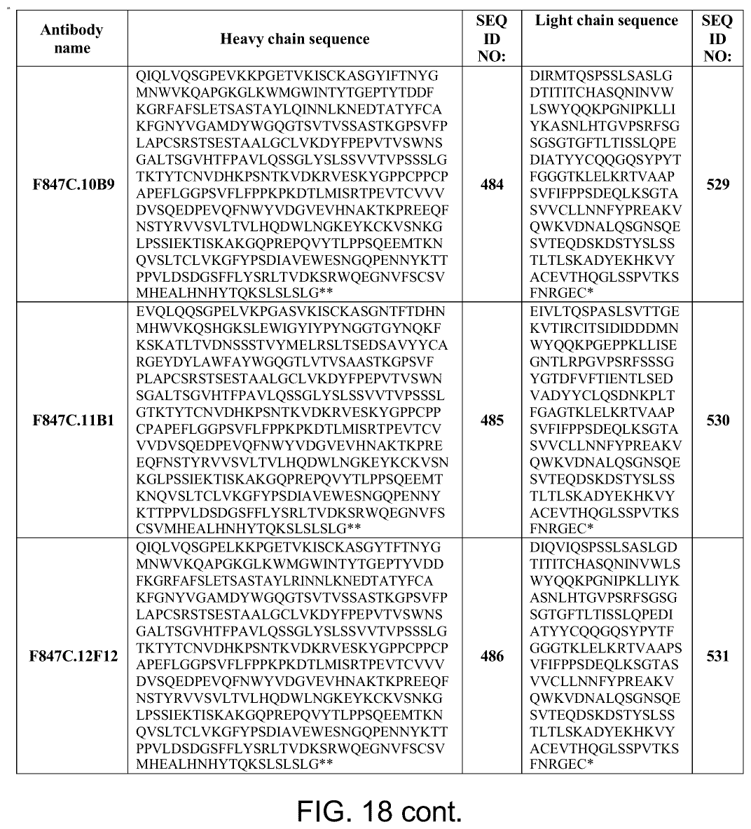

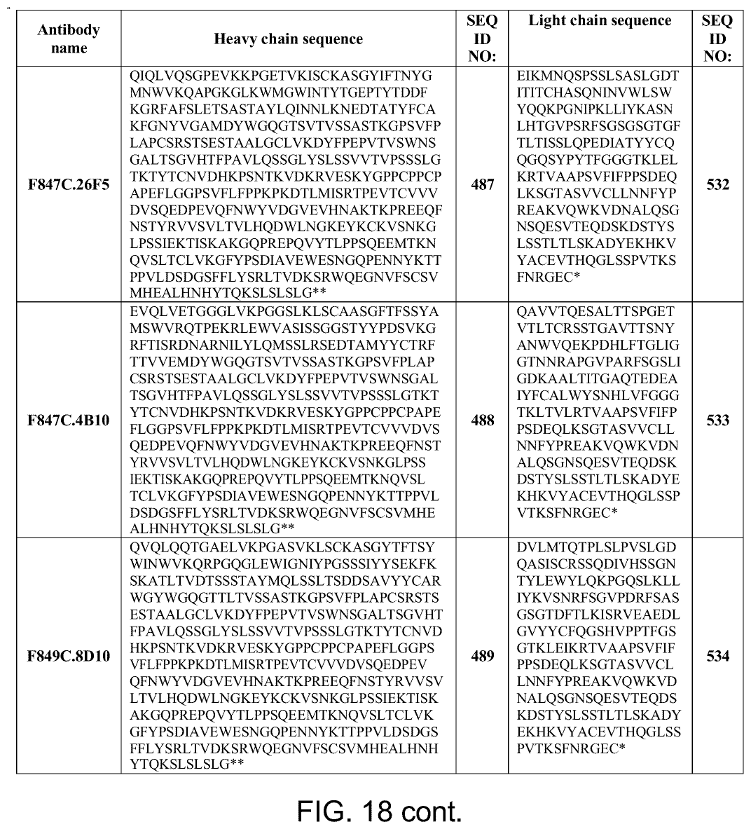

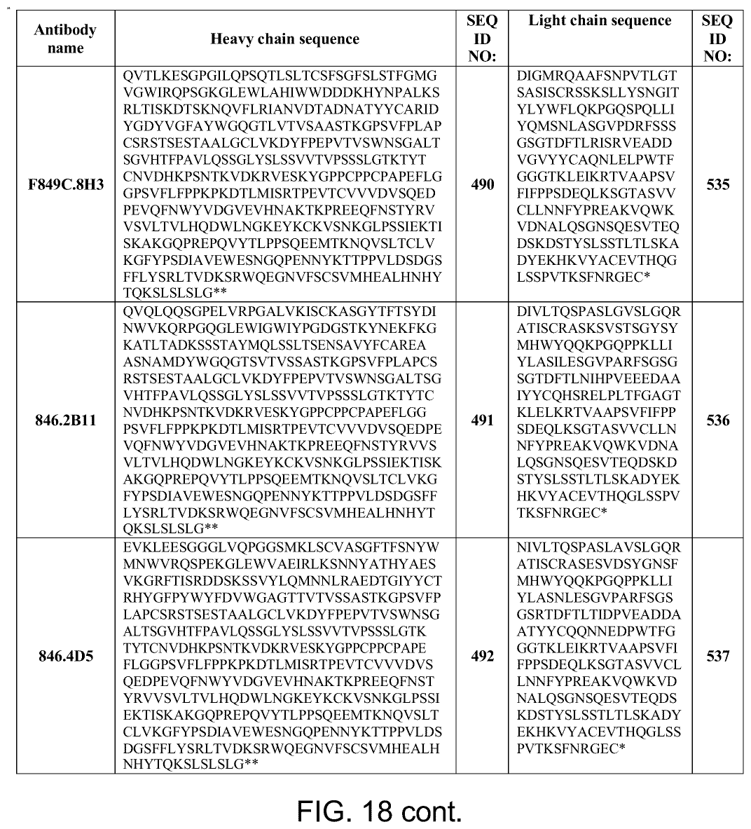

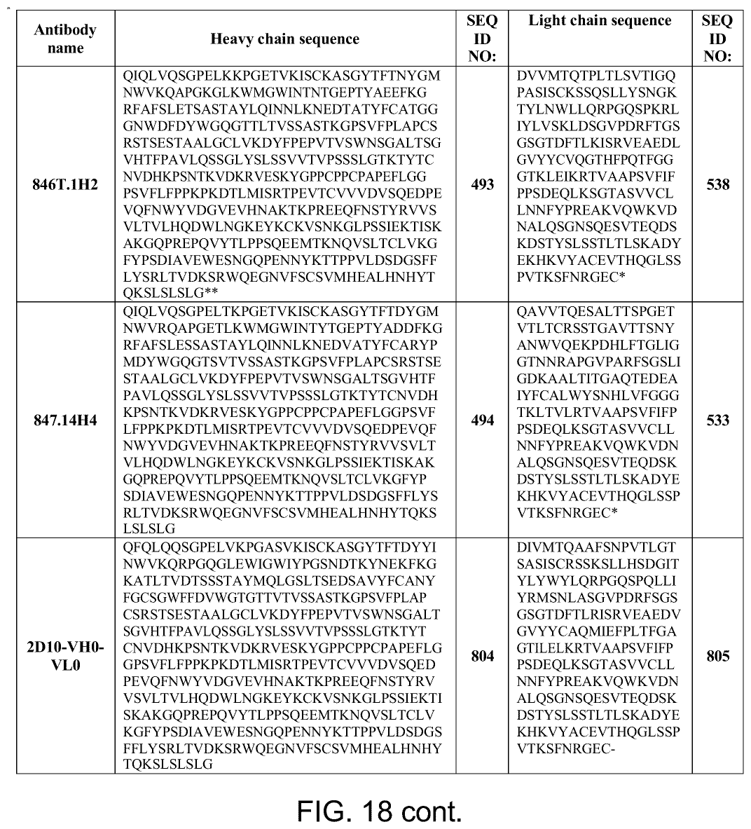

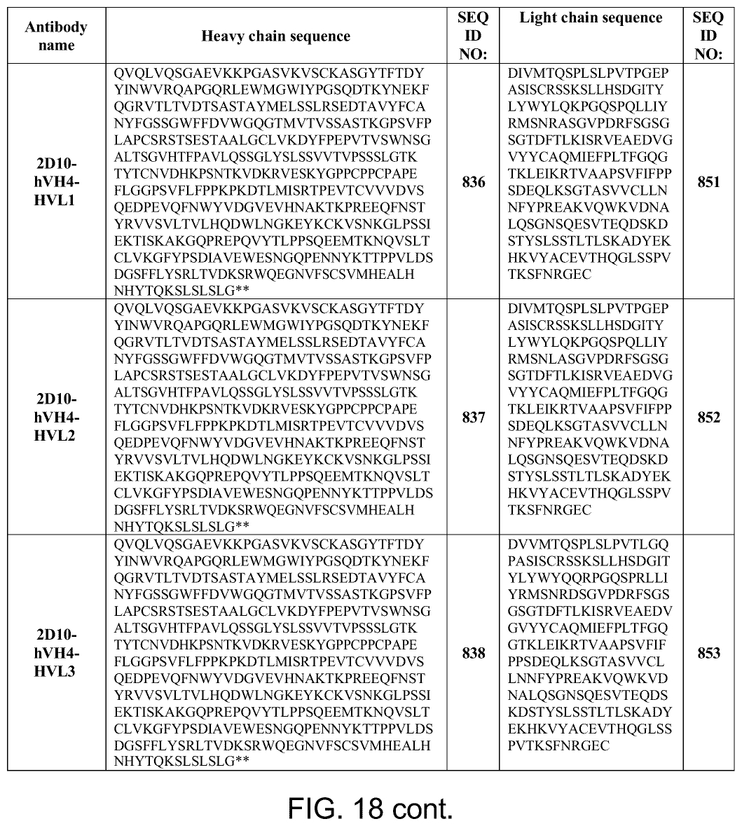

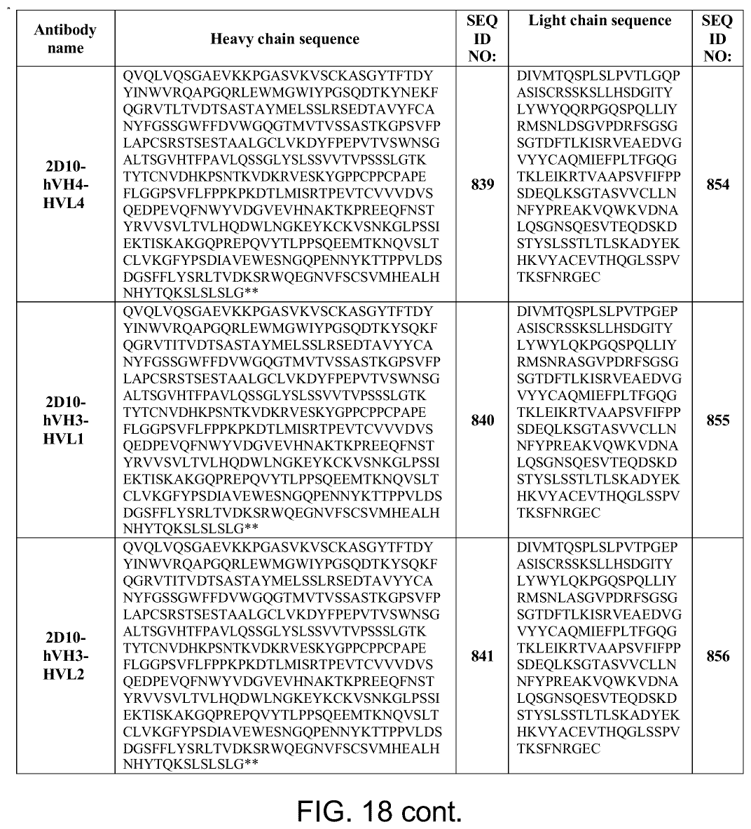

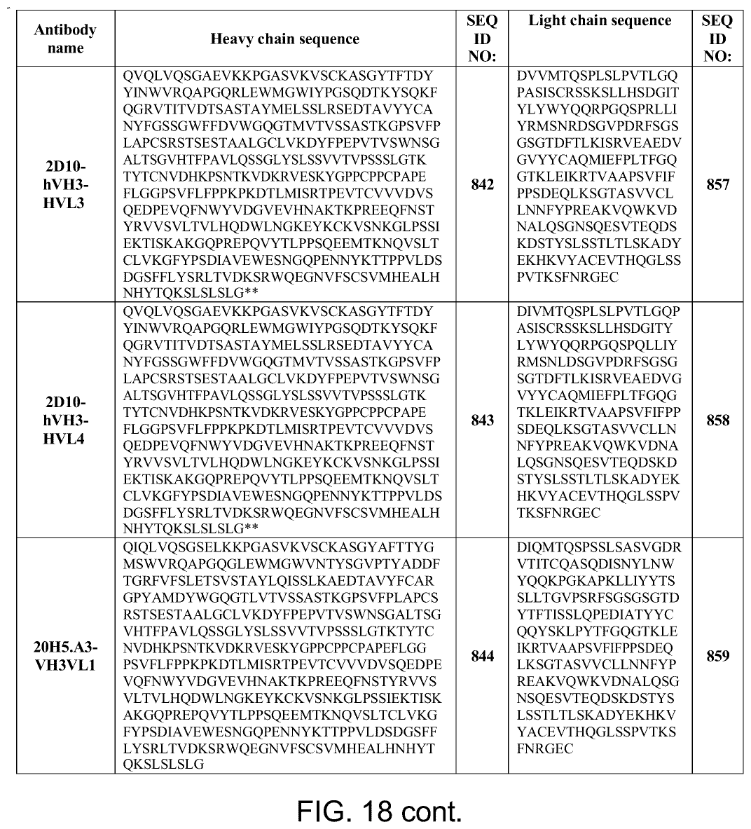

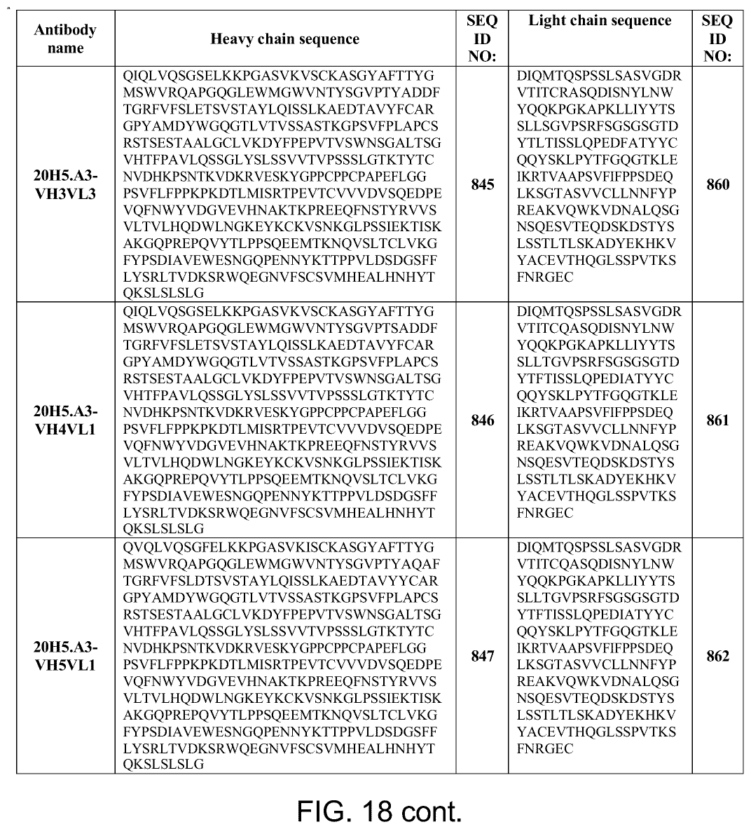

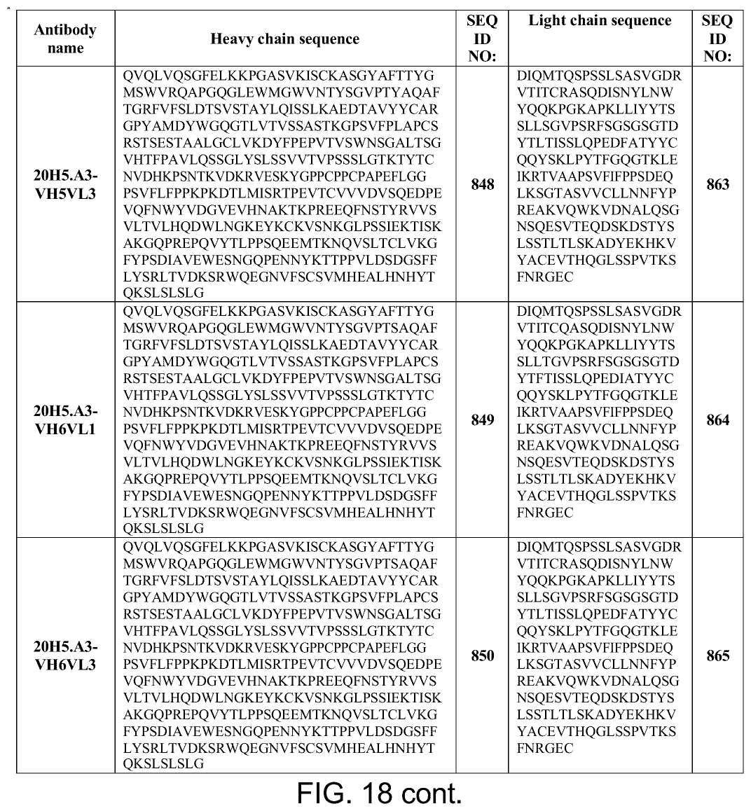

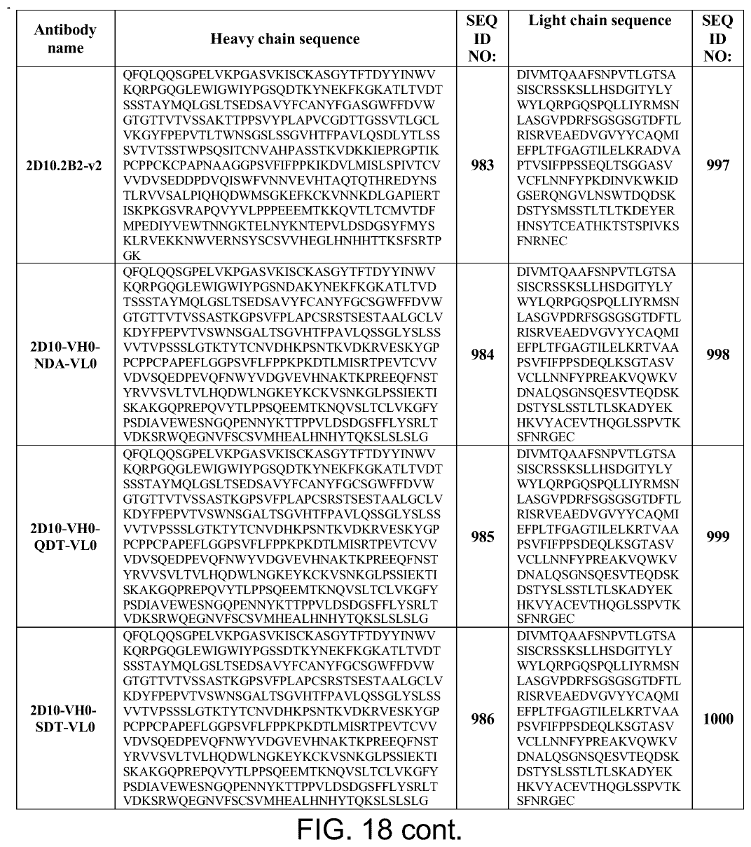

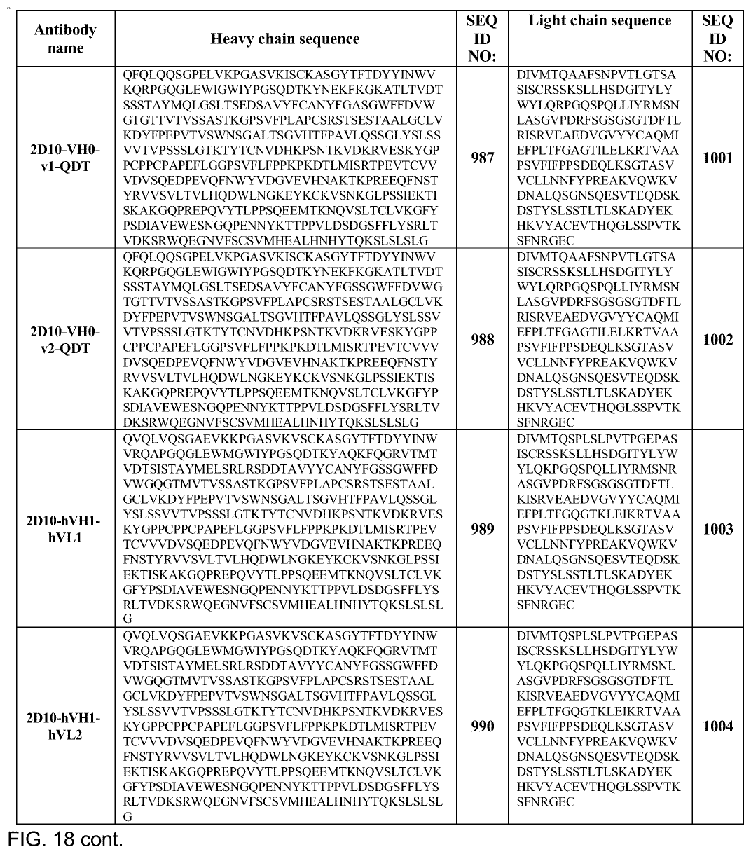

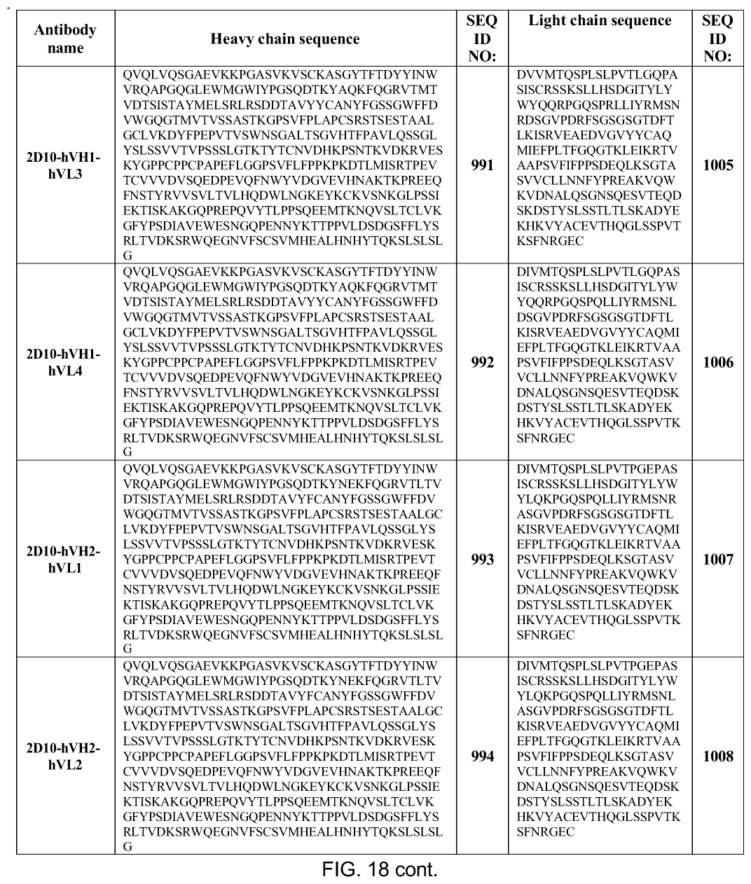

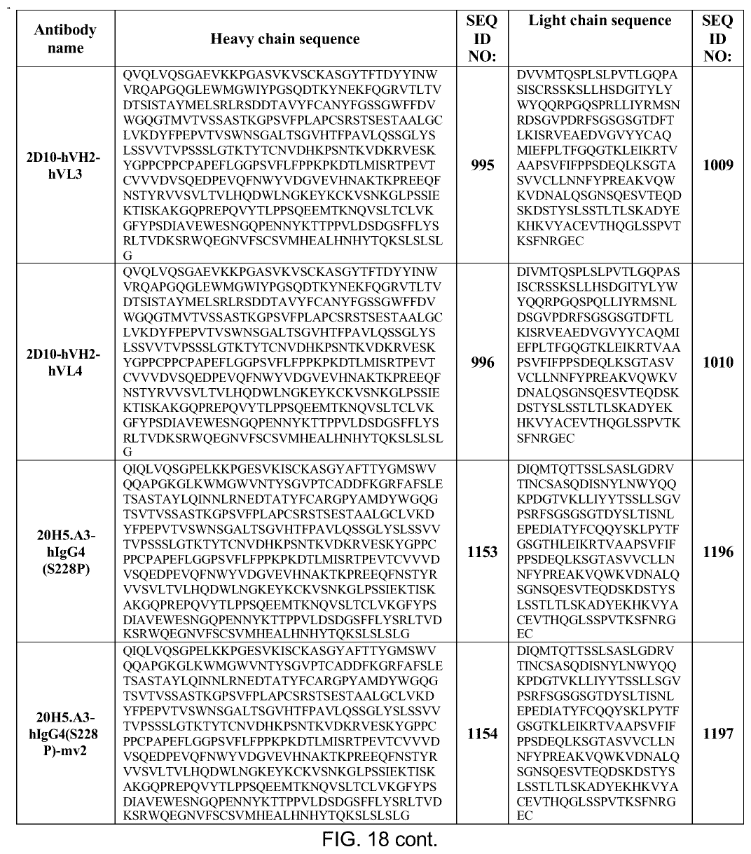

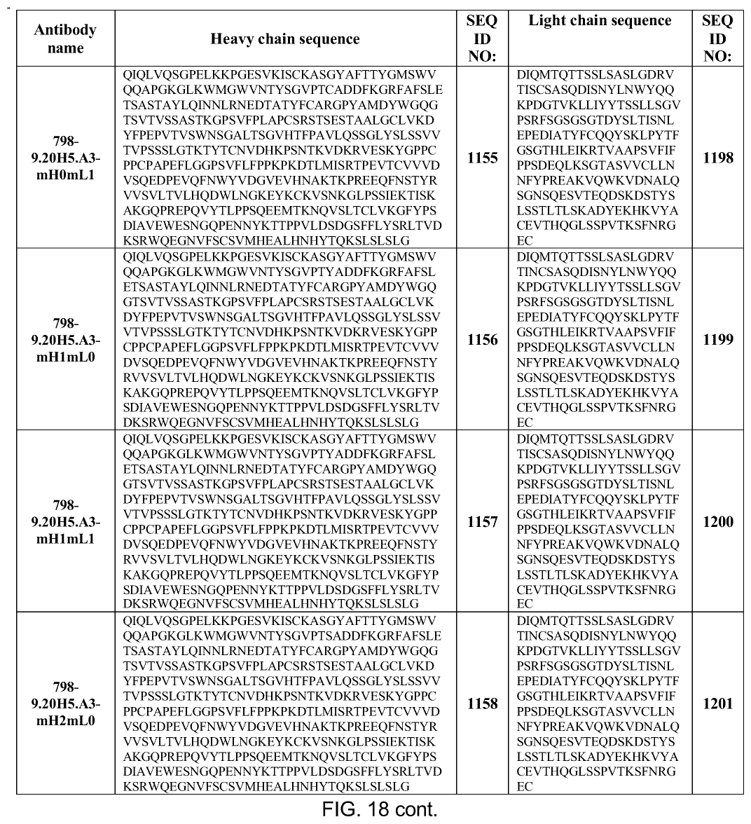

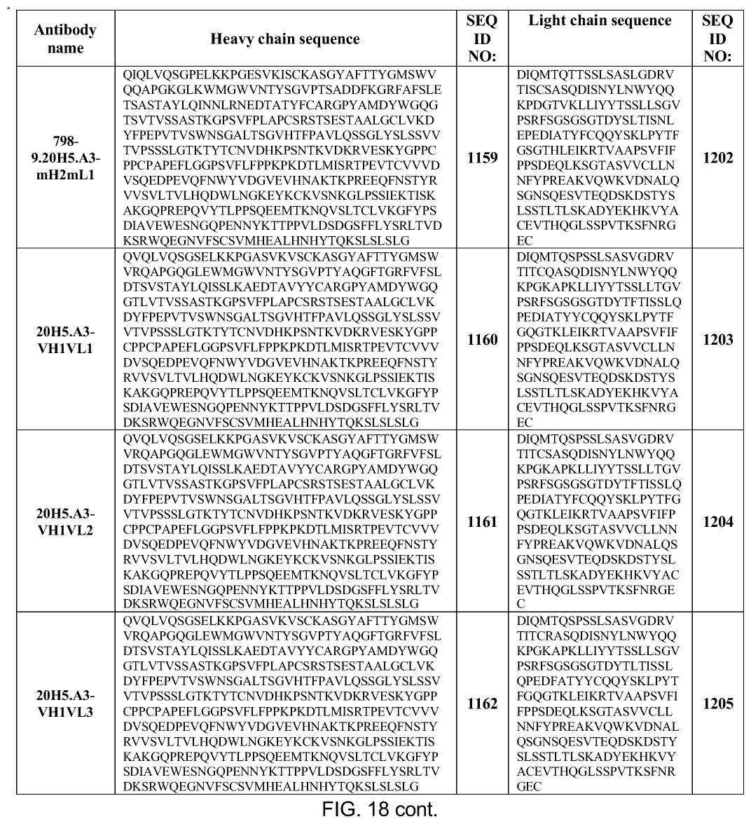

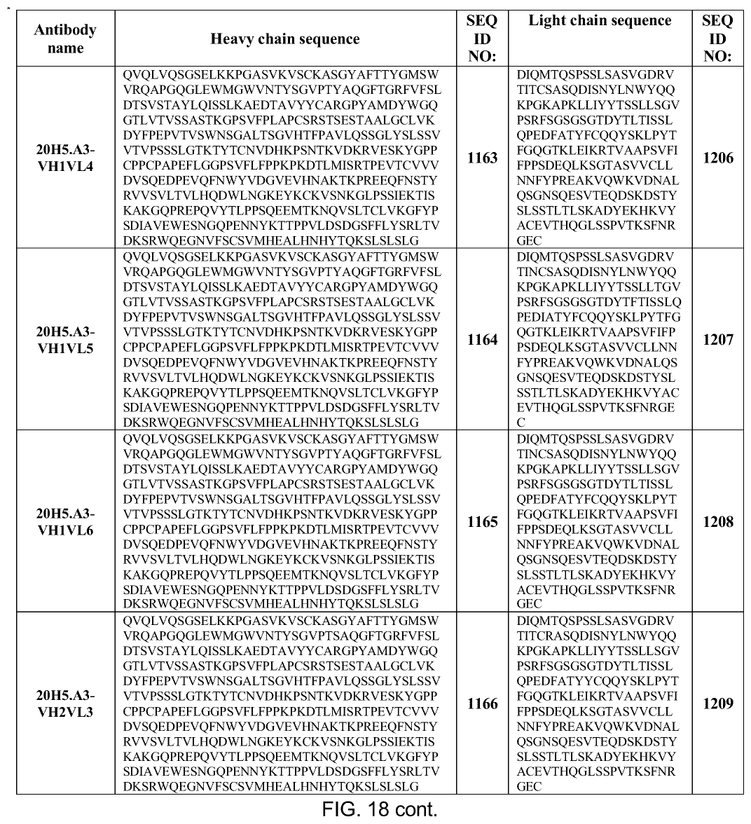

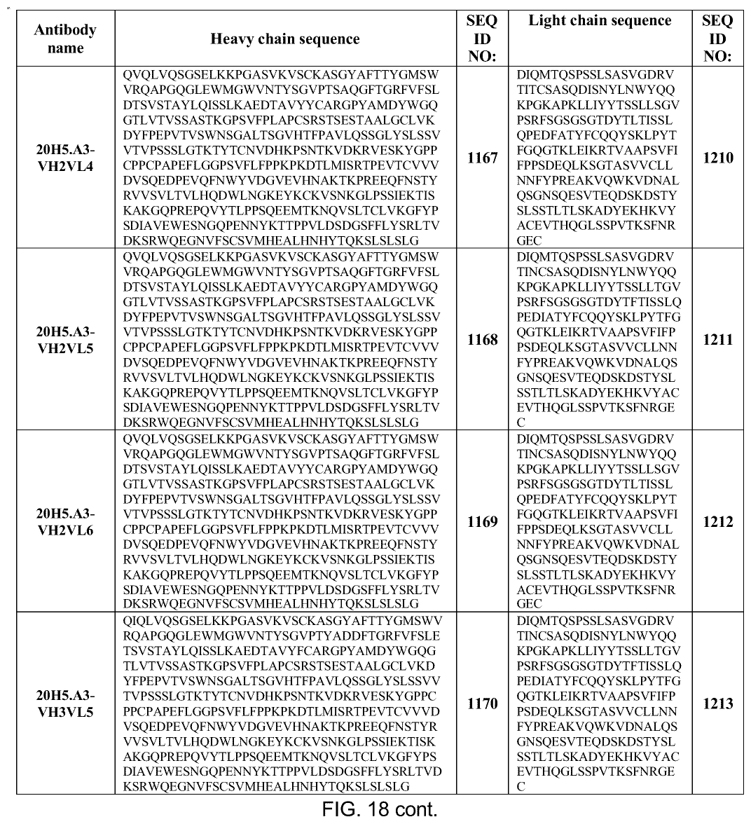

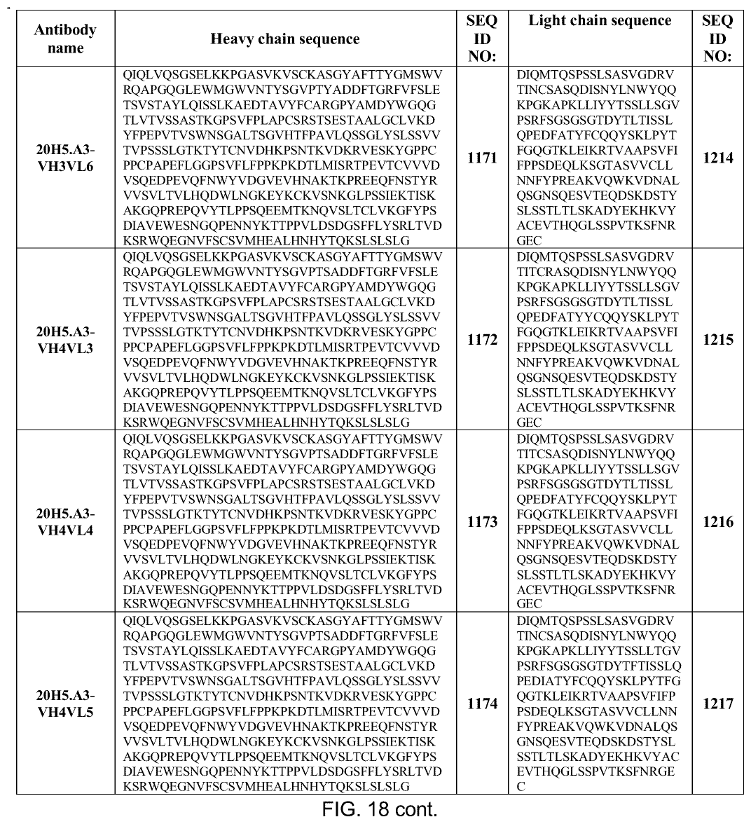

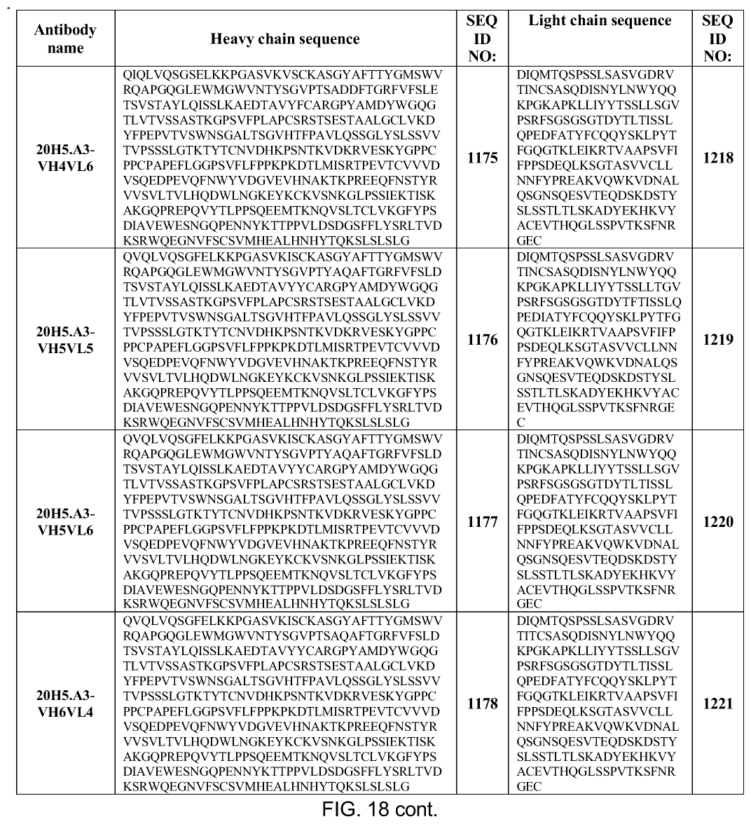

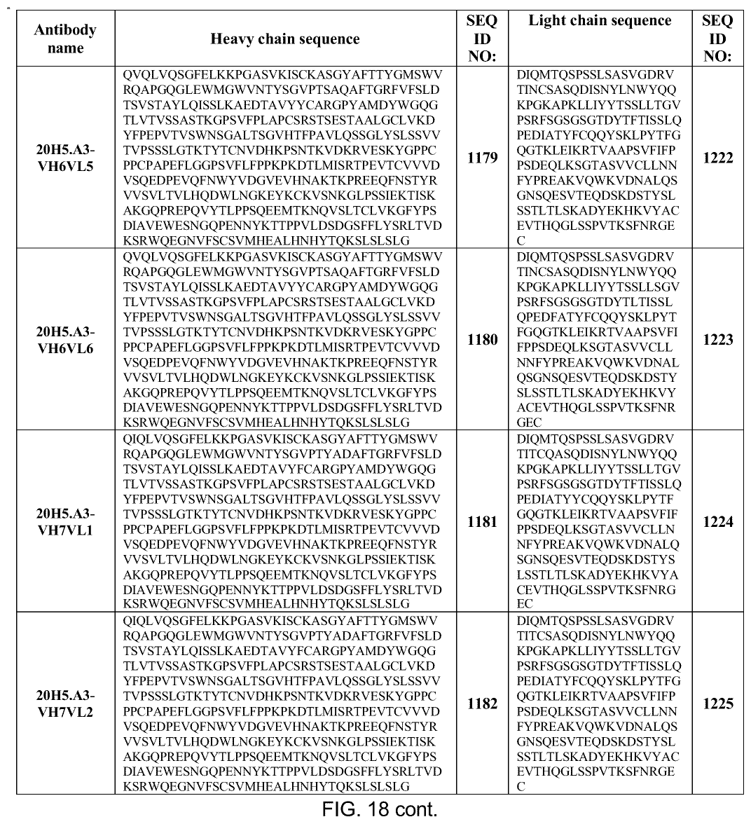

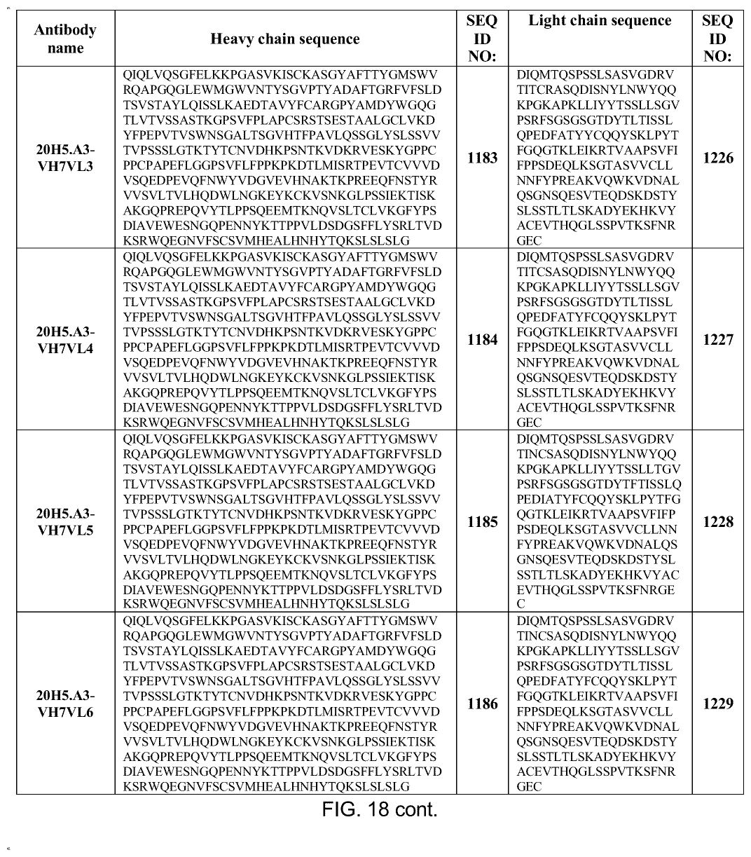

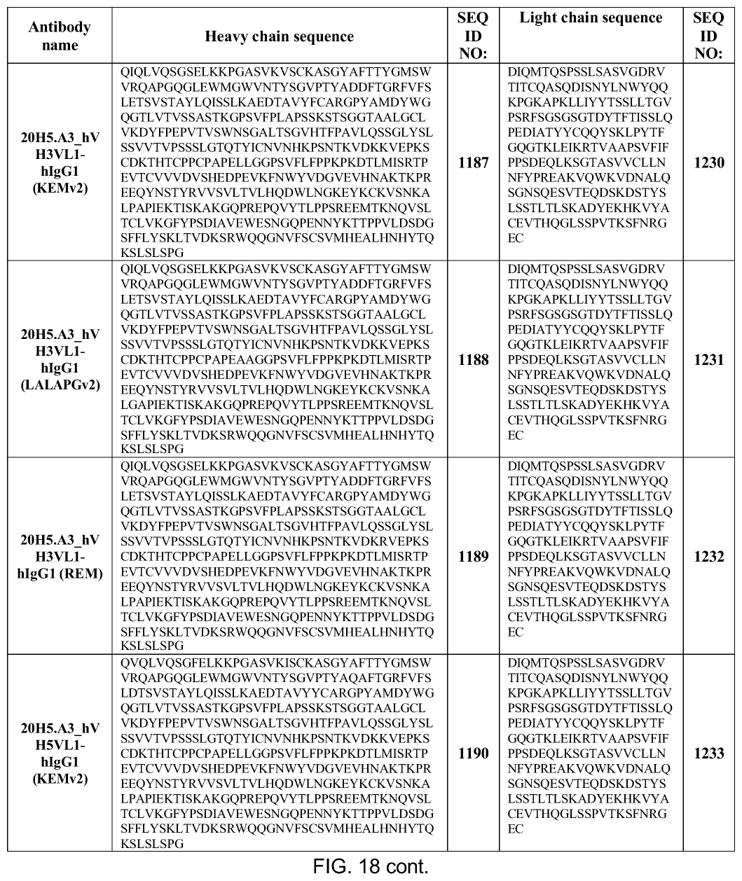

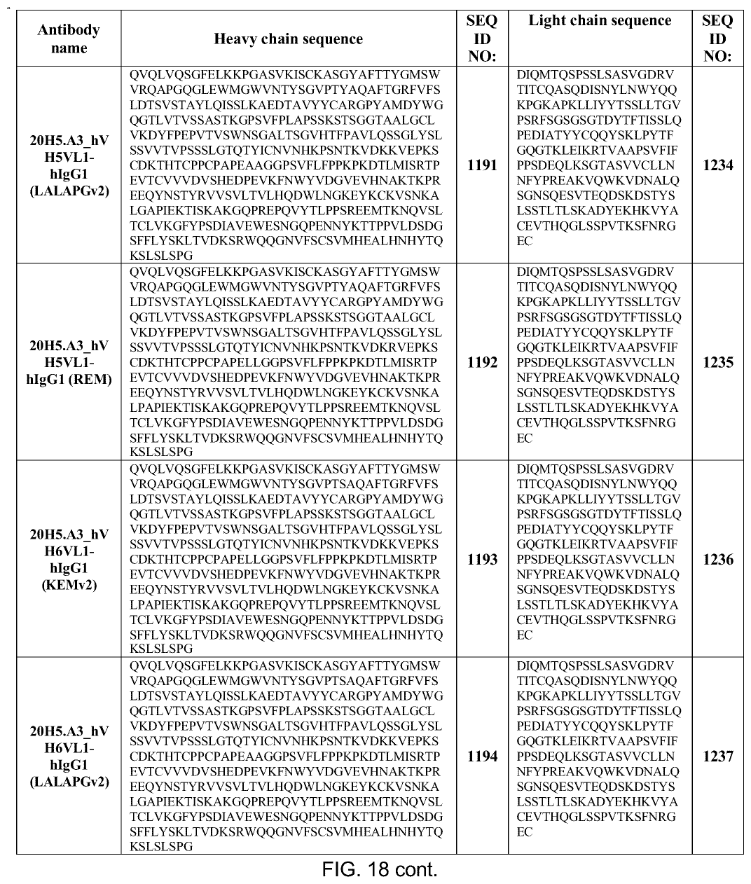

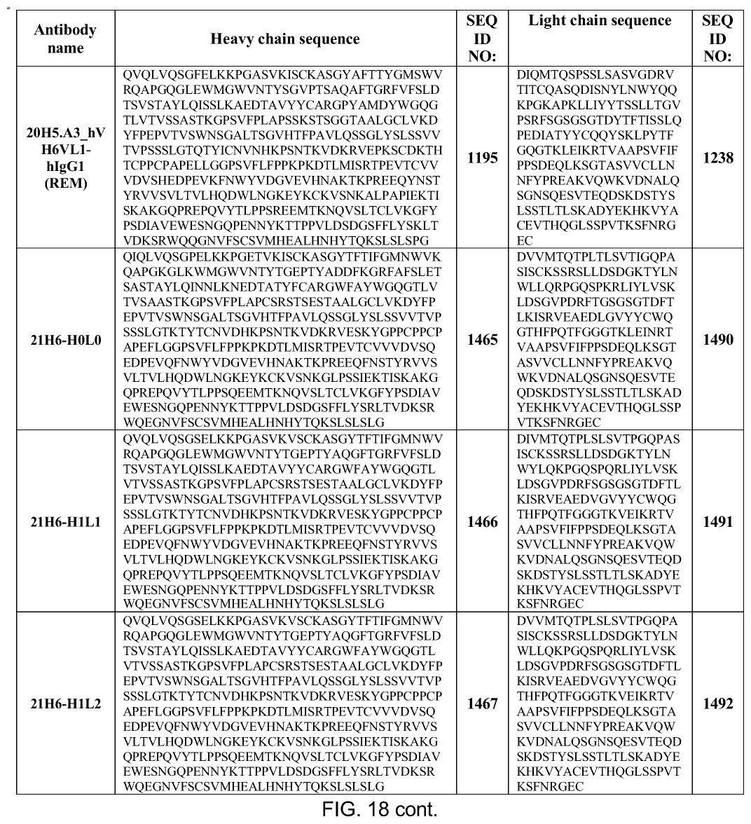

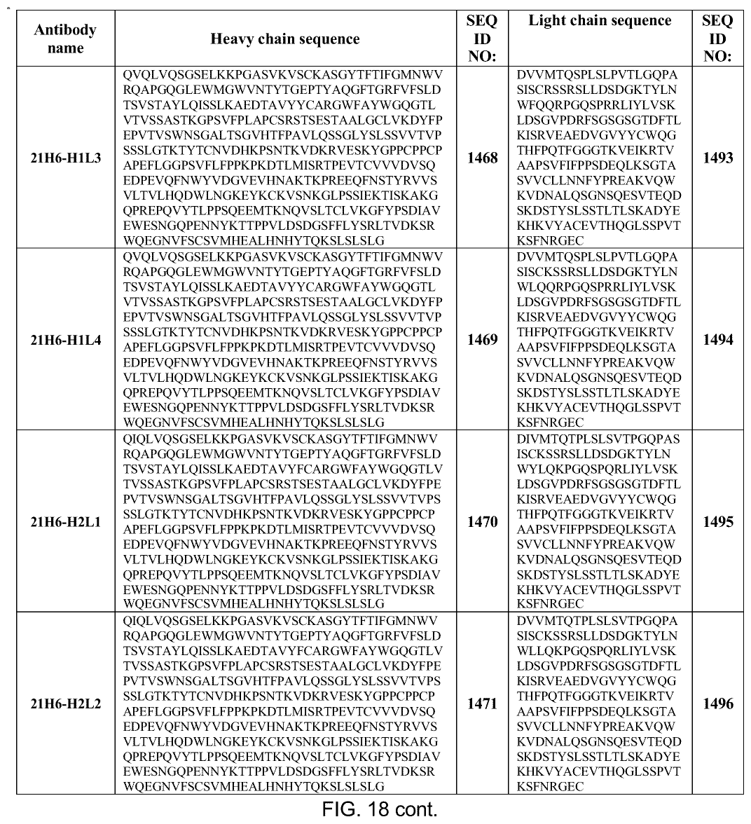

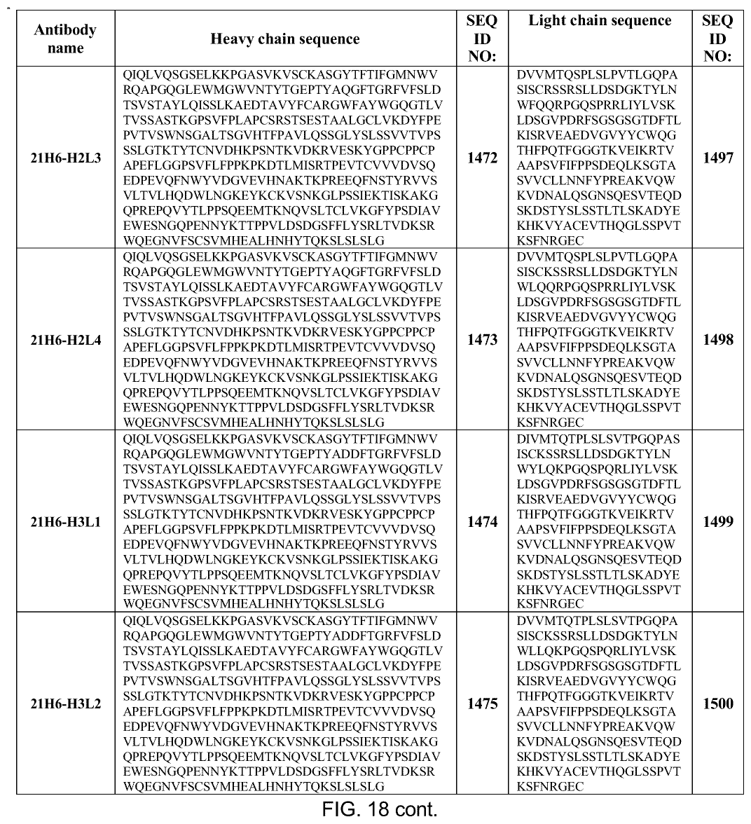

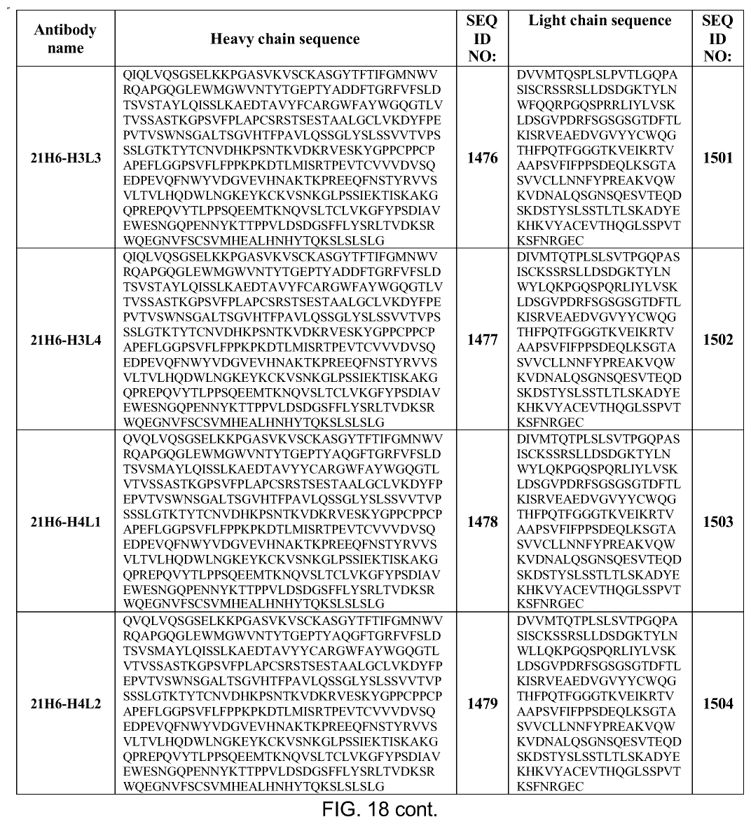

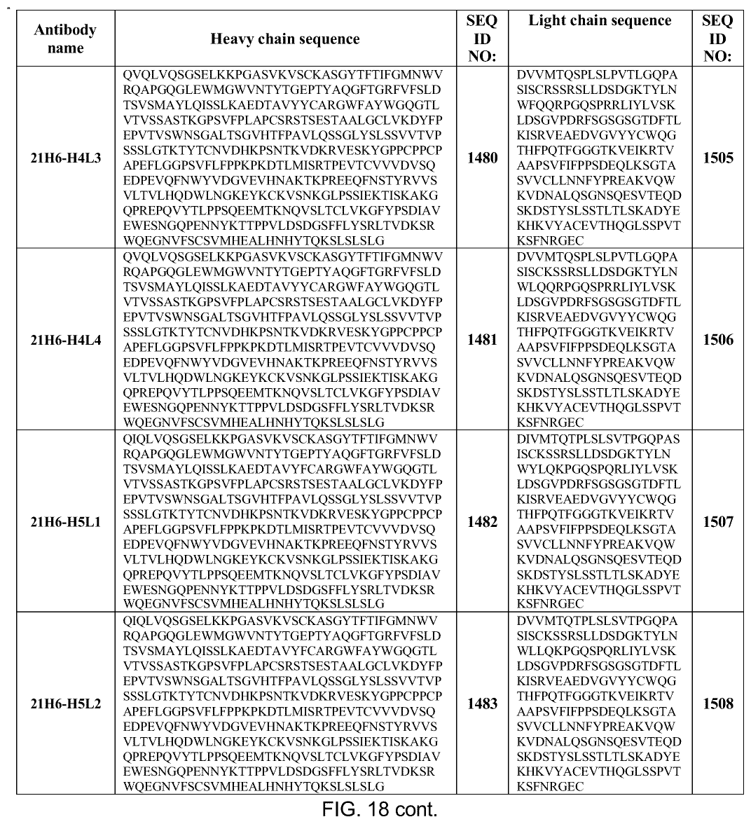

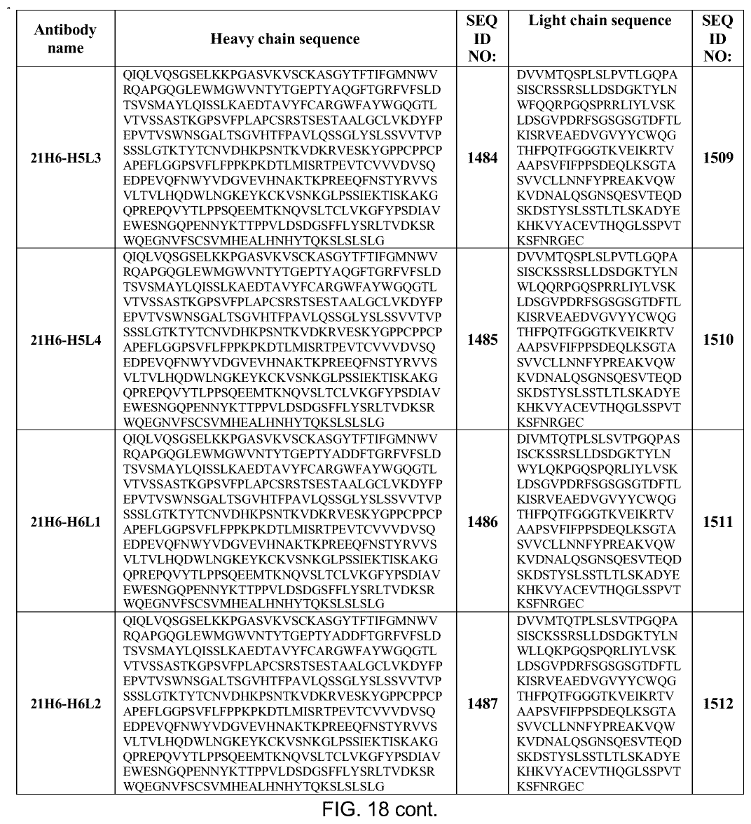

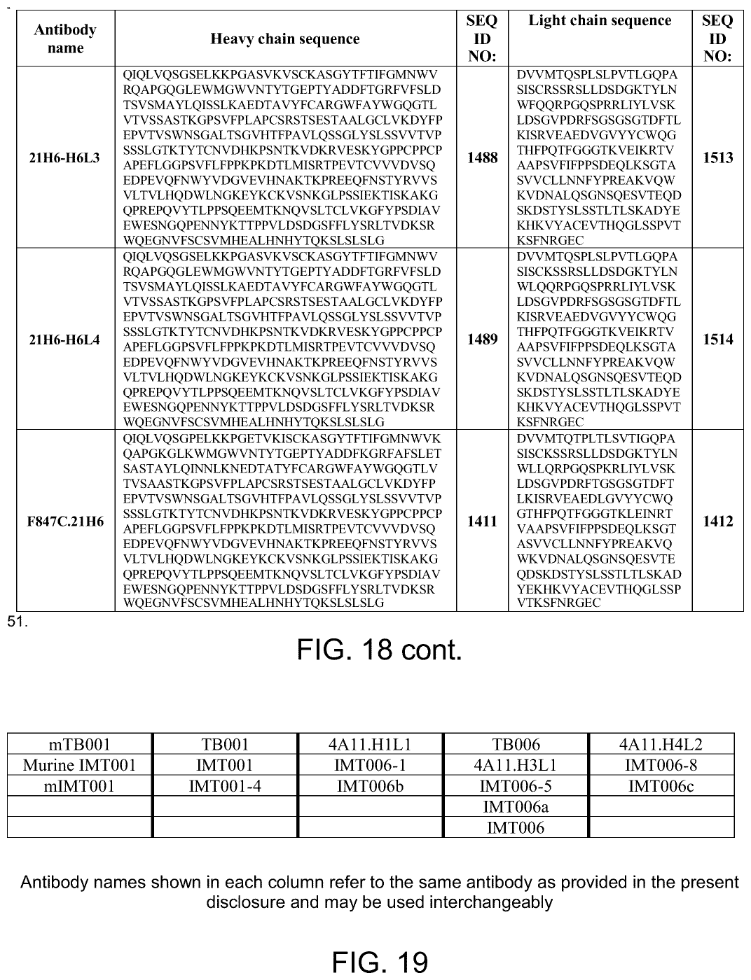

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 27-70. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 71-111, 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 112-169, 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 170-220. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 211-247. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any amino acid sequence according to SEQ ID NOs: 248-296. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 18. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 27-70. In some embodiments, the V_{H}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 71-111, 801, 951, 952. In some embodiments, the V_{H}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 112-169, 802, 953, 954. In some embodiments, the V_{L}-CDR1 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 170-220. In some embodiments, the V_{L}-CDR2 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 211-247. In some embodiments, the V_{L}-CDR3 comprises an amino acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any amino acid sequence according to SEQ ID NOs: 248-296. In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to a VL sequence, a VH sequence, a VL/VH pairing, and/or V_{H}-CDR1, V_{H}-CDR2, V_{H}-CDR3, V_{L}-CDR1, V_{L}-CDR2, V_{L}-CDR3 (including 1, 2, 3, 4, or 5 amino acid substitutions of any one or more of these CDRs) set from the heavy chain and light chain sequences as depicted in FIG. 18. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, antibodies or binding fragments thereof are provided. In some embodiments, the antibodies or binding fragments thereof are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises an amino acid sequence having at least 0, 1, 2, 3, 4, 5, or 6 substitutions relative to any amino acid sequence according to SEQ ID NOs: 27-70. In some embodiments, the V_{H}-CDR2 comprises an amino acid sequence having at least 0, 1, 2, 3, 4, 5, or 6 substitutions relative to any amino acid sequence according to SEQ ID NOs: 71-111, 801, 951, 952. In some embodiments, the V_{H}-CDR3 comprises an amino acid sequence having at least 0, 1, 2, 3, 4, 5, or 6 substitutions relative to any amino acid sequence according to SEQ ID NOs: 112-169, 802, 953, 954. In some embodiments, the V_{L}-CDR1 comprises an amino acid sequence having at least 0, 1, 2, 3, 4, 5, or 6 substitutions relative to any amino acid sequence according to SEQ ID NOs: 170-220. In some embodiments, the V_{L}-CDR2 comprises an amino acid sequence having at least 0, 1, 2, 3, 4, 5, or 6 substitutions relative to any amino acid sequence according to SEQ ID NOs: 211-247. In some embodiments, the V_{L}-CDR3 comprises an amino acid sequence having at least 0, 1, 2, 3, 4, 5, or 6 substitutions relative to any amino acid sequence according to SEQ ID NOs: 248-296. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

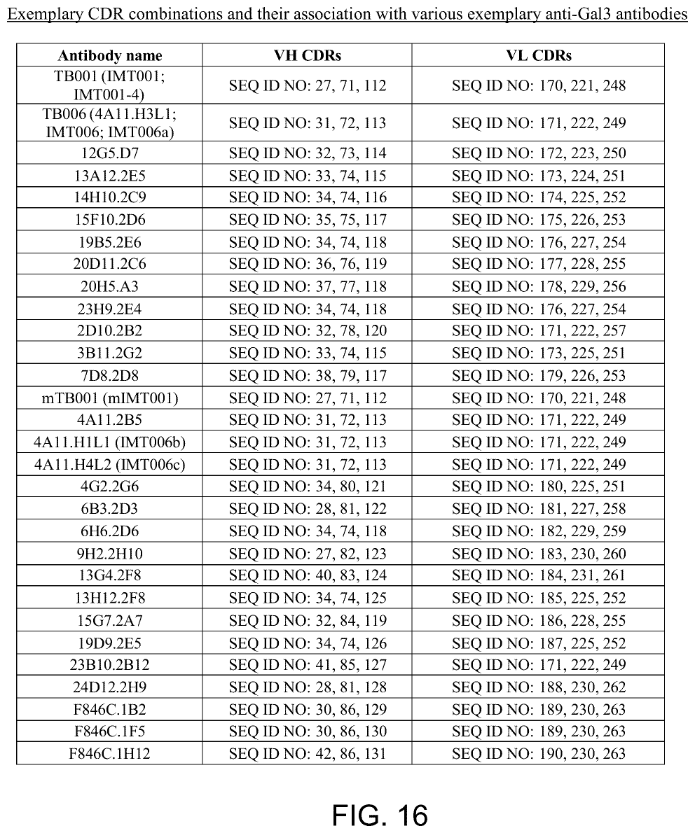

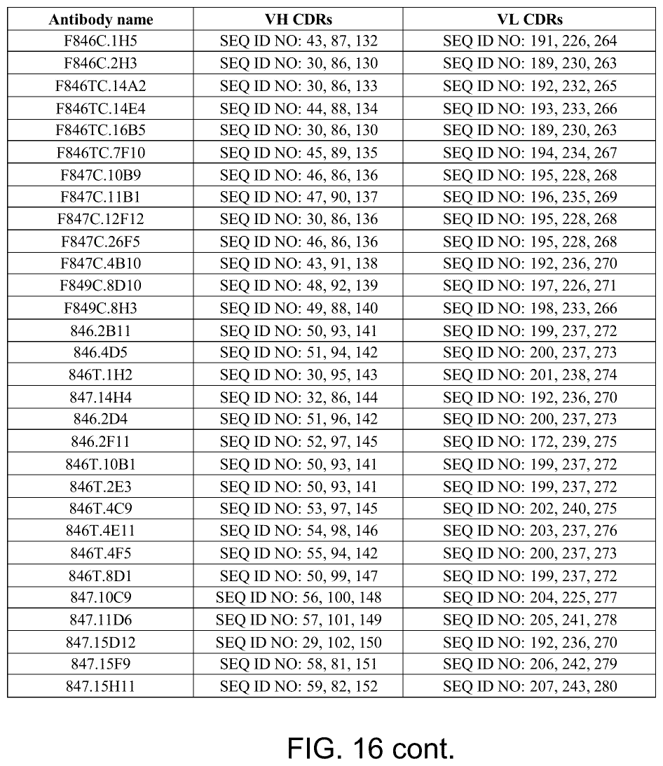

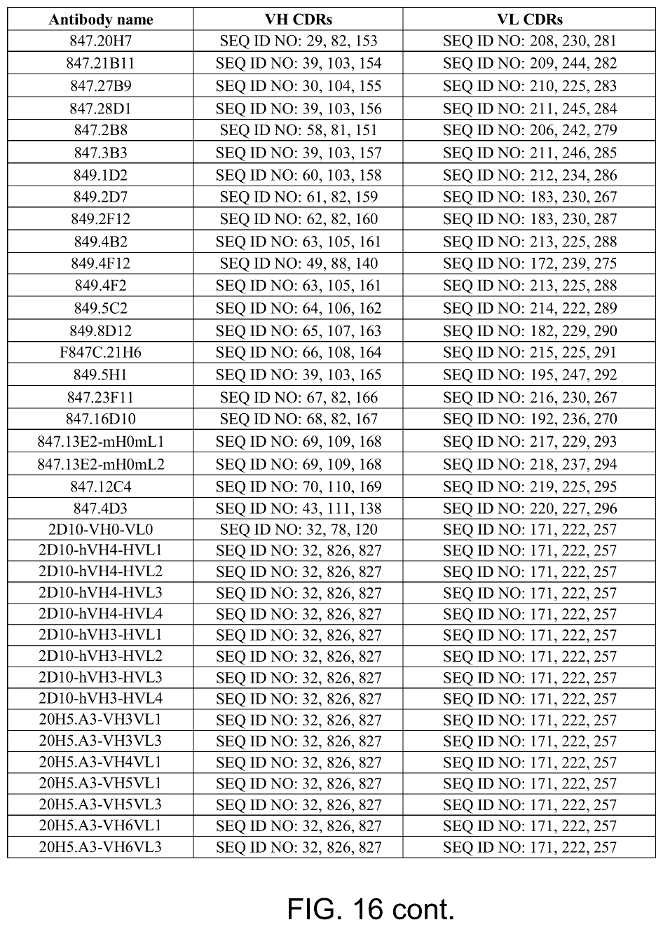

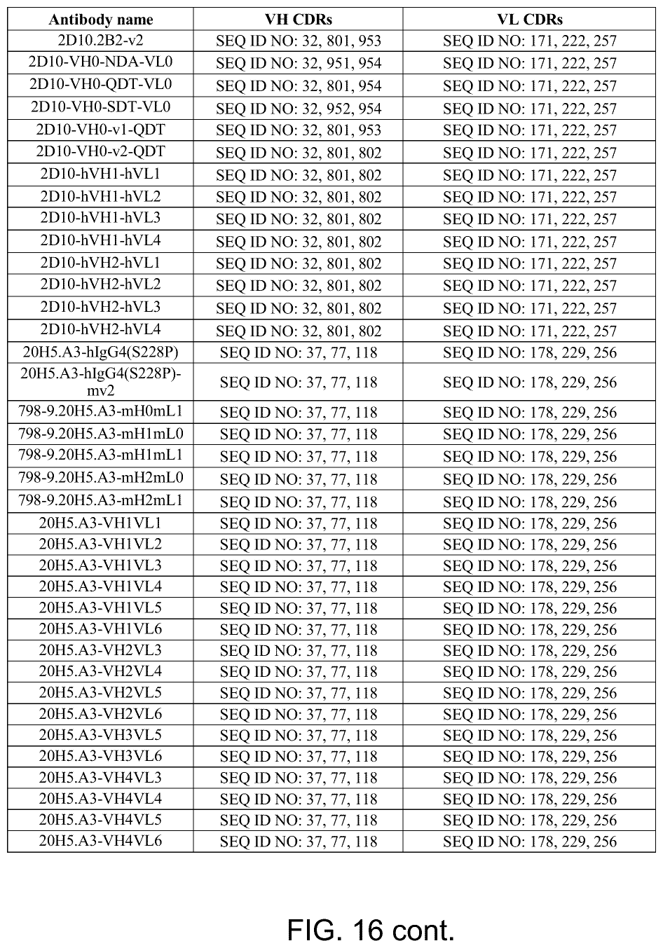

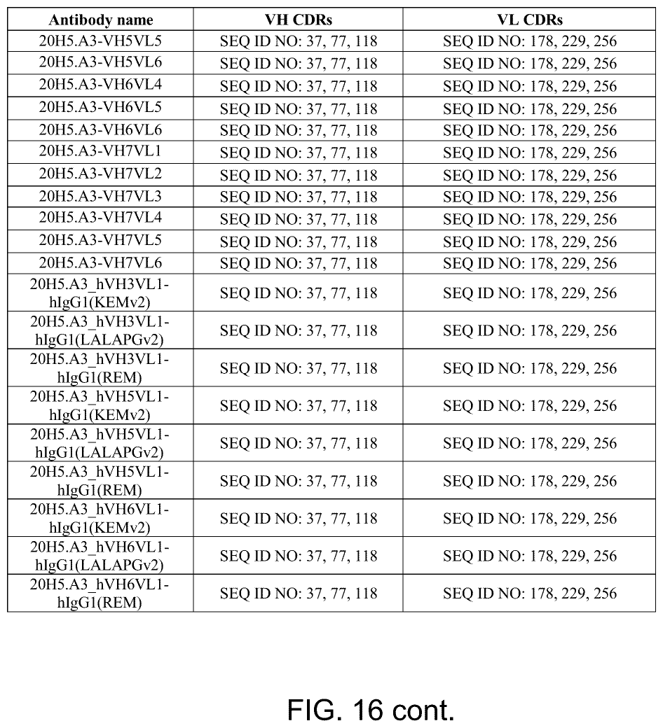

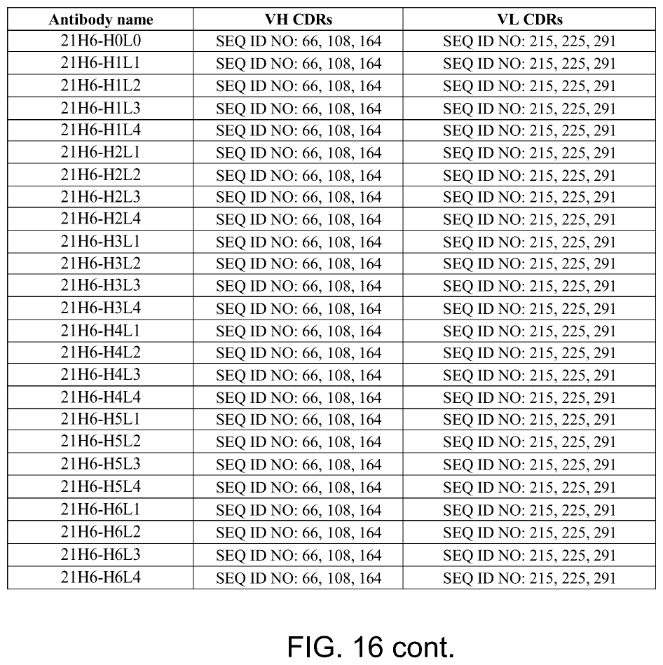

In some embodiments, the antibody or binding fragment thereof comprises a combination of a V_{L}-CDR1, a V_{L}-CDR2, a V_{L}-CDR3, a V_{H}-CDR1, a V_{H}-CDR2, and a V_{H}-CDR3 as illustrated in FIG. 16.

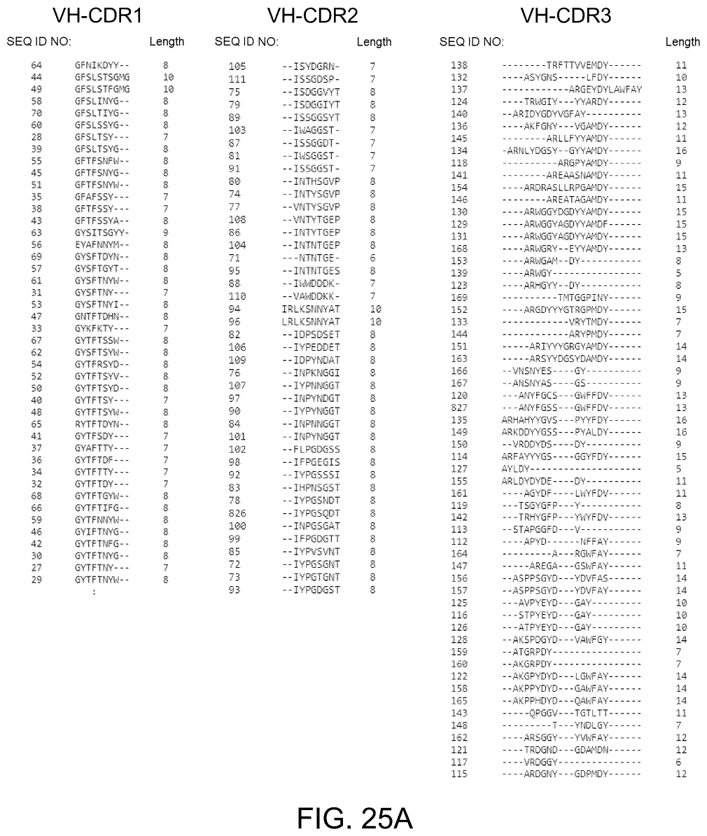

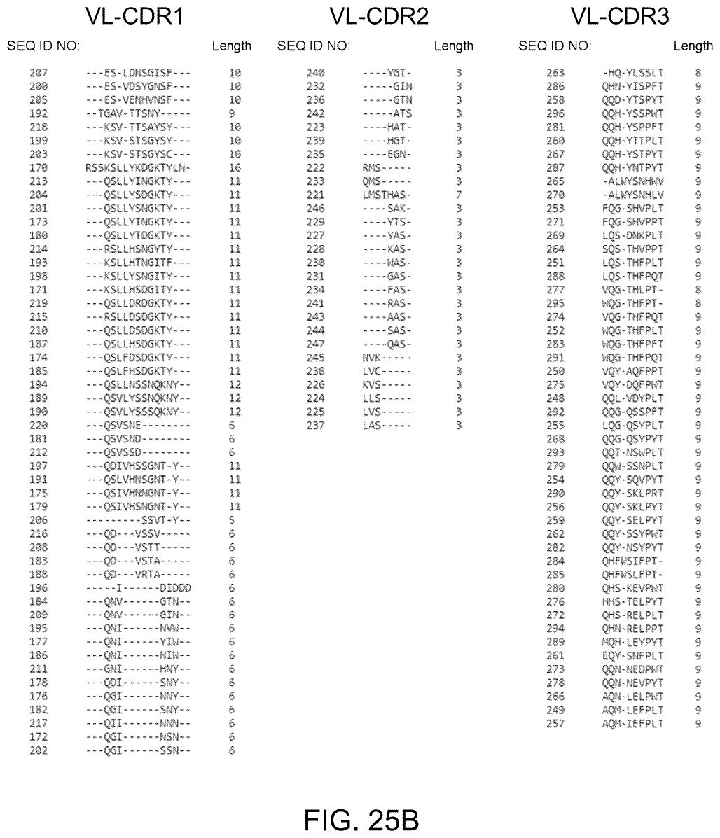

In some embodiments, the antibody or binding fragment thereof comprises a combination of a V_{H}-CDR1, a V_{H}-CDR2, a V_{H}-CDR3, V_{L}-CDR1, a V_{L}-CDR2, and a V_{L}-CDR3, where one or more of these CDRs is defined by a consensus sequence. The consensus sequences provided herein have been derived from the alignments of CDRs depicted in FIG. 25A-B. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the V_{H}-CDR1 is defined by the formula X₁X₂X₃X₄X₅X₆X₇X₈X₉X₁₀, where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the V_{H}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR1 comprises a sequence having 0, 1, 2, 3, 4, 5, or 6 substitutions from this consensus sequence.

In some embodiments, the V_{H}-CDR2 is defined by the formula X₁X₂X₃X₄X₅X₆X₇X₈X₉X₁₀, where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the V_{H}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR2 comprises a sequence having 0, 1, 2, 3, 4, 5, or 6 substitutions from this consensus sequence.

In some embodiments, the V_{H}-CDR3 is defined by the formula X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅, where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X₁₇ is no amino acid, F, I, L, M, R, or Y; X₁₈ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the V_{H}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{H}-CDR3 comprises a sequence having 0, 1, 2, 3, 4, 5, or 6 substitutions from this consensus sequence.

In some embodiments, the V_{L}-CDR1 is defined by the formula X₁X₂X₃X₄X₅X₆X₇X₈X₉X₁₀X₁₁X₁₂X₁₃X₁₄X₁₅X₁₆X₁₇, where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; X₈ is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q, T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the V_{L}-CDR1 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR1 comprises a sequence having 0, 1, 2, 3, 4, 5, or 6 substitutions from this consensus sequence.

In some embodiments, the V_{L}-CDR2 is defined by the formula X₁X₂X₃X₄X₅X₆X₇X₈, where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; X₈ is no amino acid, N, or S. In some embodiments, the V_{L}-CDR2 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR2 comprises a sequence having 0, 1, 2, 3, 4, 5, or 6 substitutions from this consensus sequence.

In some embodiments, the V_{L}-CDR3 is defined by the formula X₁X₂X₃X₄X₅X₆X₇X₈X₉X₁₀, where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P, S, T, V, W, or Y; X₈ is H, P, or S; X₉ is F, L, P, Q, R, T, W, or Y; X₁₀ is no amino acid, T, or V. In some embodiments, the V_{L}-CDR3 comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the V_{L}-CDR3 comprises a sequence having 0, 1, 2, 3, 4, 5, or 6 substitutions from this consensus sequence.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 297-373, 803, 806-820, 955-968, 1067-1109, 1415-1439. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 374-447, 821-835, 941-943, 969-982, 1110-1152, 1440-1464. In some embodiments, the antibodies or binding fragments thereof are anti-Gal3 antibodies or binding fragments thereof.

In some embodiments, the heavy chain variable region of the anti-Gal3 antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 297-373, 803, 806-820, 955-968, 1067-1109, 1415-1439. In some embodiments, the light chain variable region of the antibody or binding fragment thereof comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 374-447, 821-835, 941-943, 969-982, 1110-1152, 1440-1464. In some embodiments, the antibodies or binding fragments thereof are anti-Gal3 antibodies or binding fragments thereof.

In some embodiments, the antibodies comprise one or more sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to a VL sequence, a VH sequence, a VL/VH pairing, and/or V_{L}-CDR1, V_{L}-CDR2, V_{L}-CDR3, V_{H}-CDR1, V_{H}-CDR2, V_{H}-CDR3 (including 1, 2, 3, 4, or 5 amino acid substitutions of any one or more of these CDRs) set from the heavy chain and light chain sequences as depicted in FIG. 18.

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprises a heavy chain variable region comprising a V_{H}-CDR1, a V_{H}-CDR2, and a V_{H}-CDR3. In some embodiments, the anti-Gal3 antibodies or binding fragments thereof comprise a light chain variable region comprising a V_{L}-CDR1, a V_{L}-CDR2, and a V_{L}-CDR3. In some embodiments, the V_{H}-CDR1 comprises one of the amino acid sequences of SEQ ID NOs: 27-70, the V_{H}-CDR2 comprises one of the amino acid sequences of SEQ ID NOs: 71-111, 801, 951, 952, the V_{H}-CDR3 comprises one of the amino acid sequences of SEQ ID NO: 112-169, 802, 953, 954, the V_{L}-CDR1 comprises one of the amino acid sequences of SEQ ID NOs: 170-220, the V_{L}-CDR2 comprises one of the amino acid sequences of SEQ ID NOs: 211-247, the V_{L}-CDR3 comprises one of the amino acid sequences of SEQ ID NOs: 248-296, the heavy chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 374-447, 821-835, 969-982, 1110-1152, 1440-1464, and the light chain variable region has a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one of the amino acid sequences of SEQ ID NOs: 374-447, 821-835, 927-929.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence selected from SEQ ID NOs: 448-494, 804, 836-850, 983-996, 1153-1195, 1465-1489. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1490-1514. In some embodiments, the antibodies or binding fragments thereof are anti-Gal3 antibodies or binding fragments thereof.

In some embodiments, the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% similarity to the sequence selected from SEQ ID NOs: 448-494, 804, 836-850, 983-996, 1153-1195, 1465-1489. In some embodiments, the antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% similarity to the sequence selected from SEQ ID NOs: 495-538, 805, 851-865, 997-1010, 1196-1238, 1490-1514. In some embodiments, the antibodies or binding fragments thereof are anti-Gal3 antibodies or binding fragments thereof.

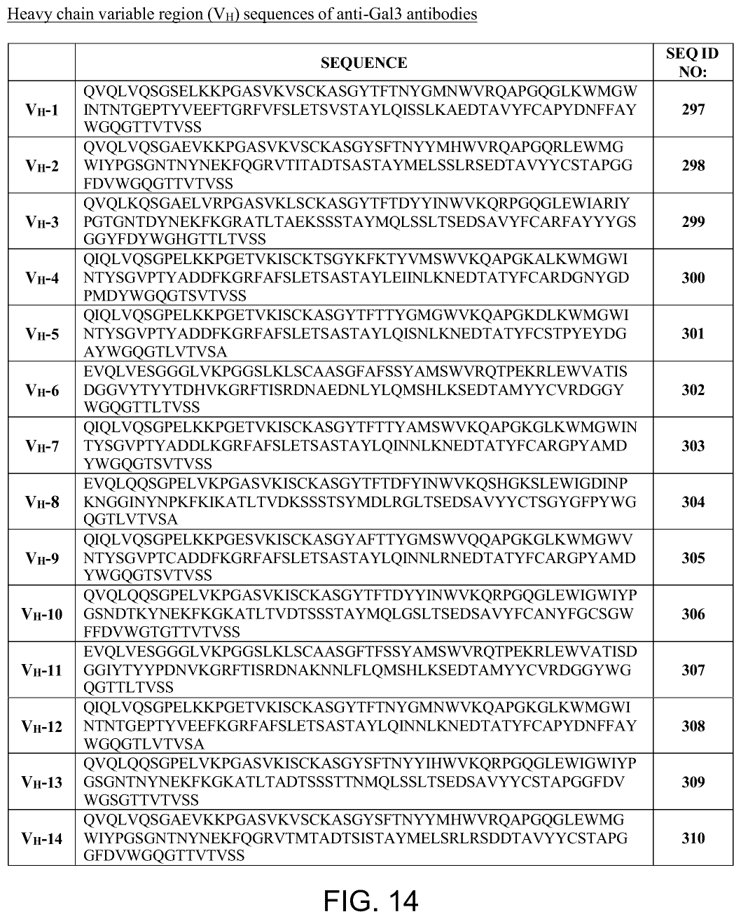

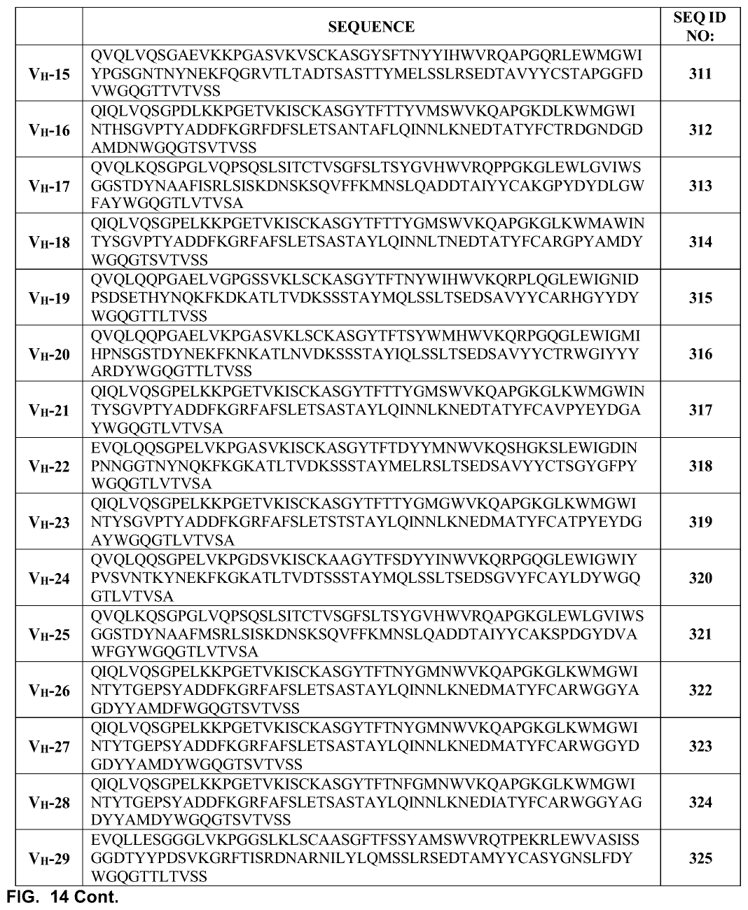

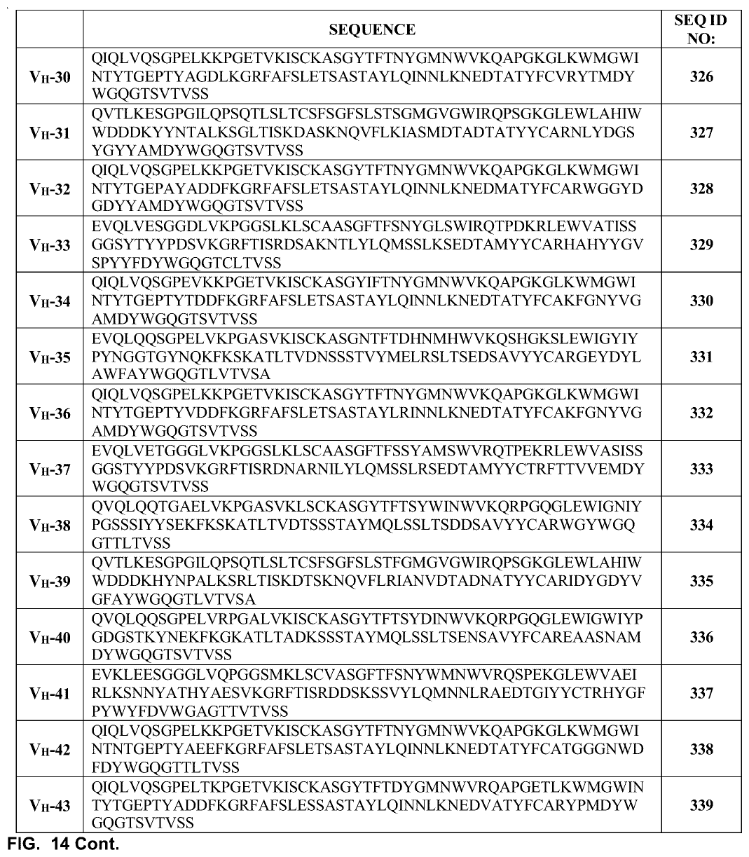

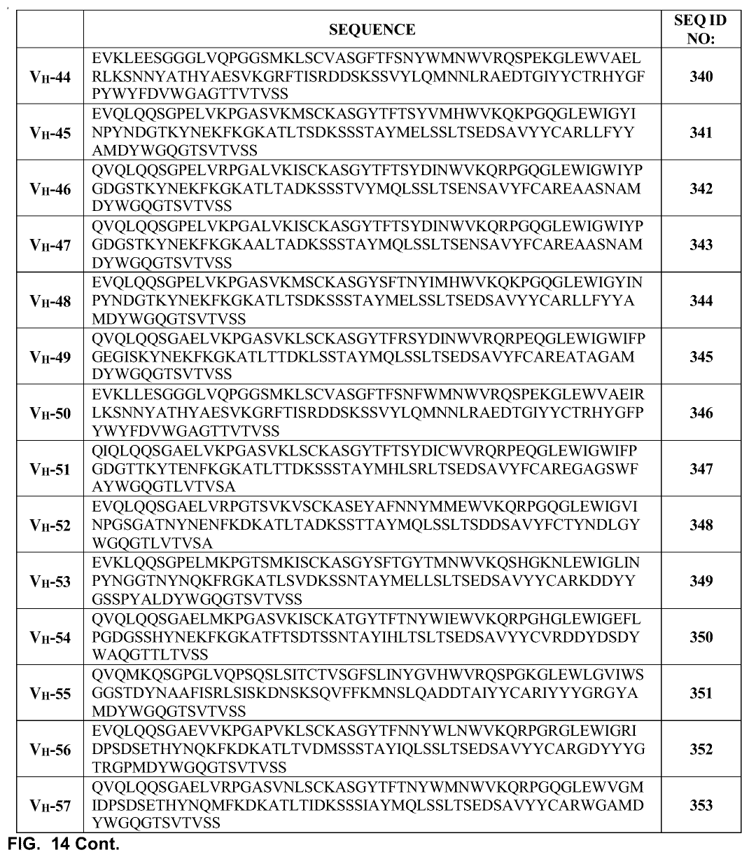

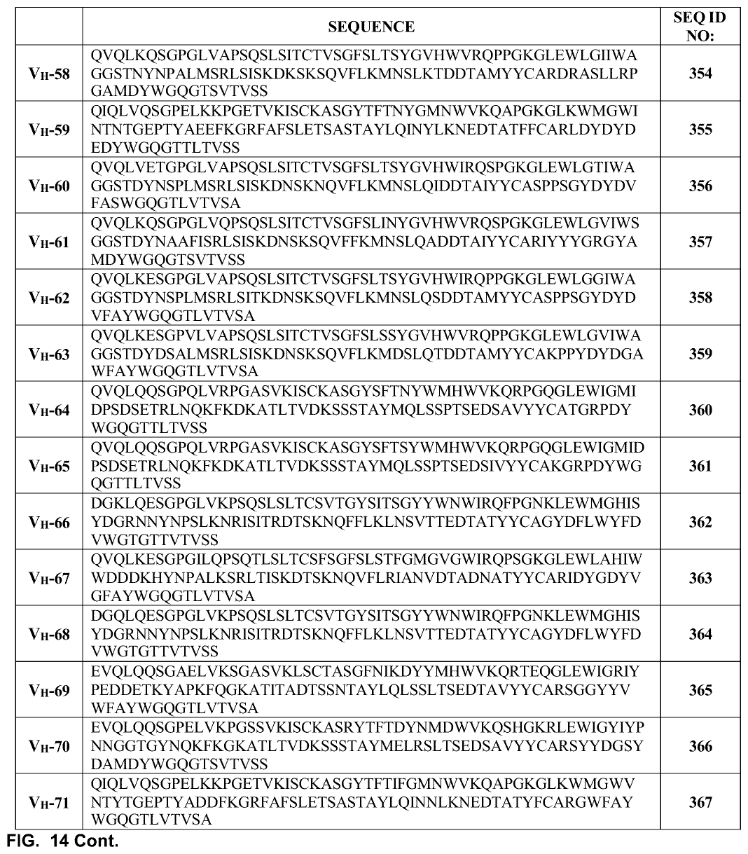

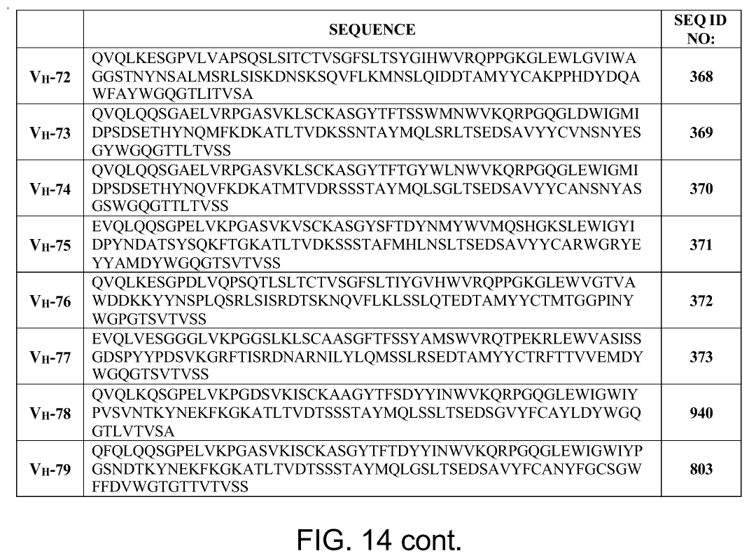

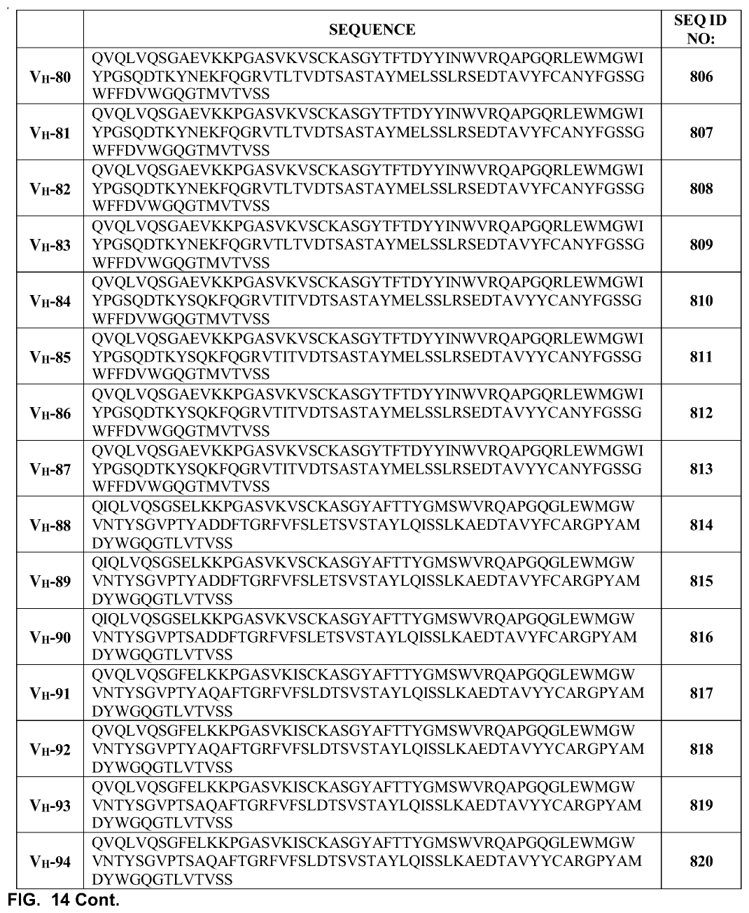

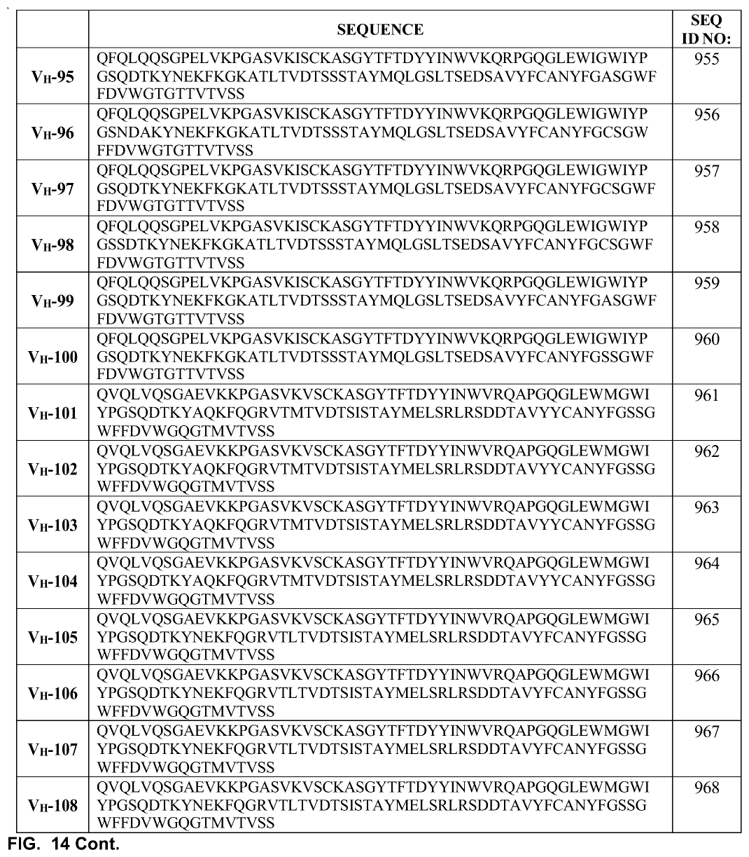

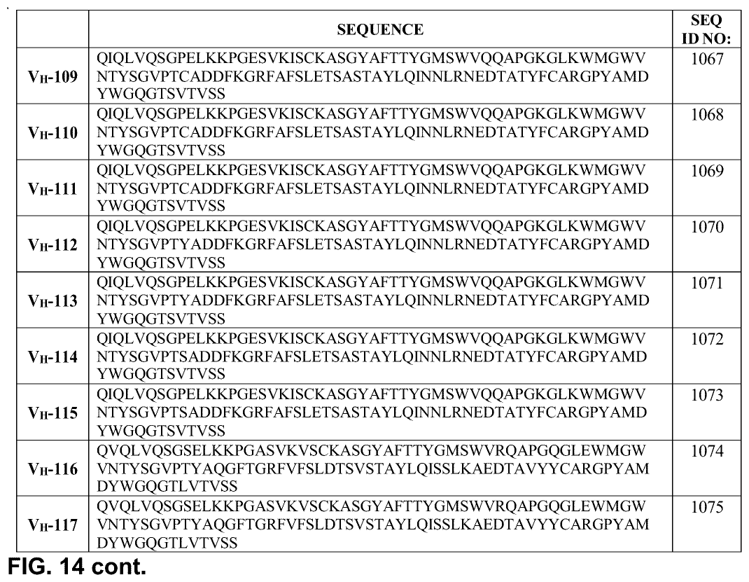

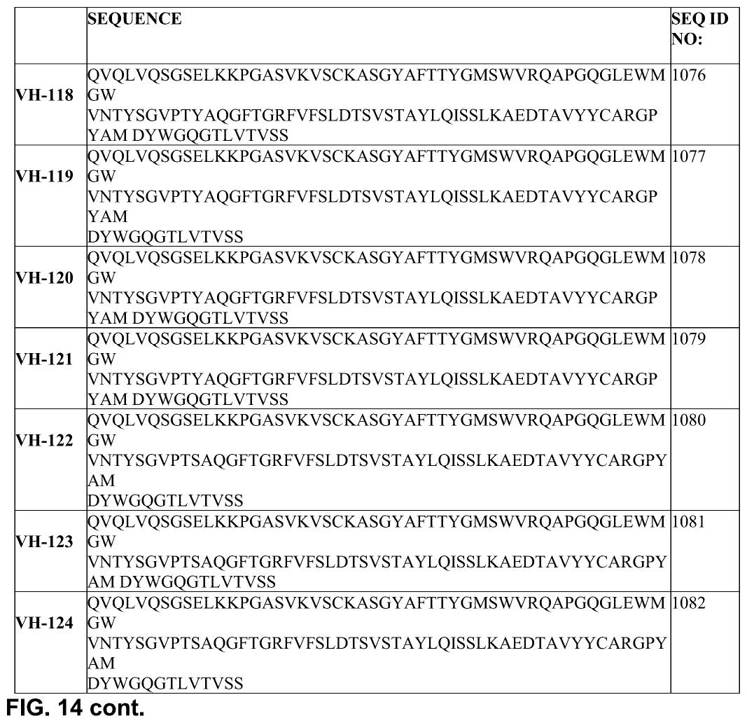

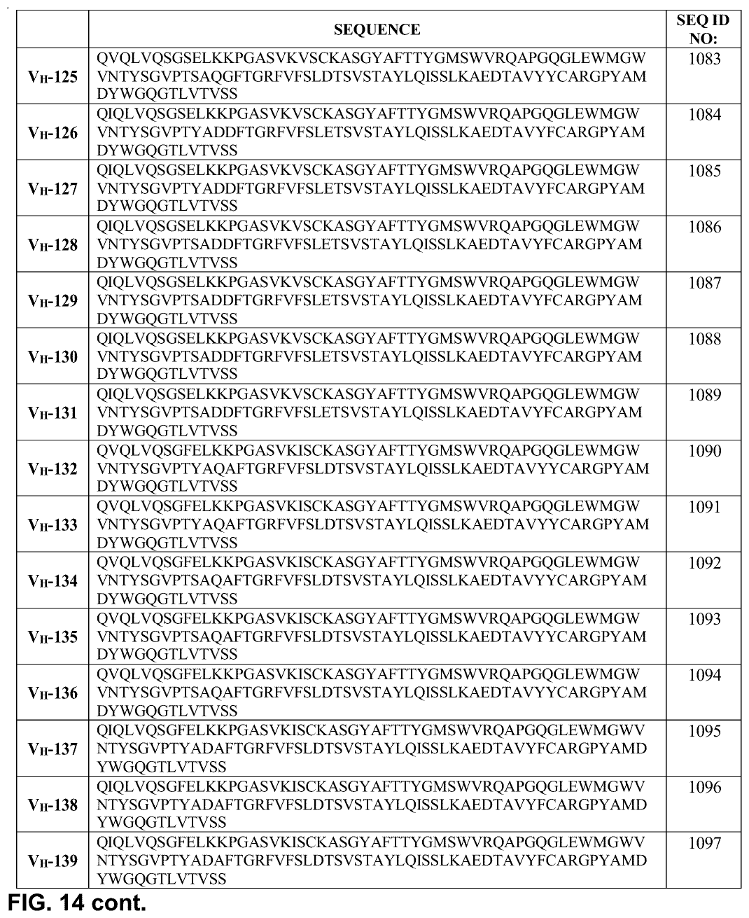

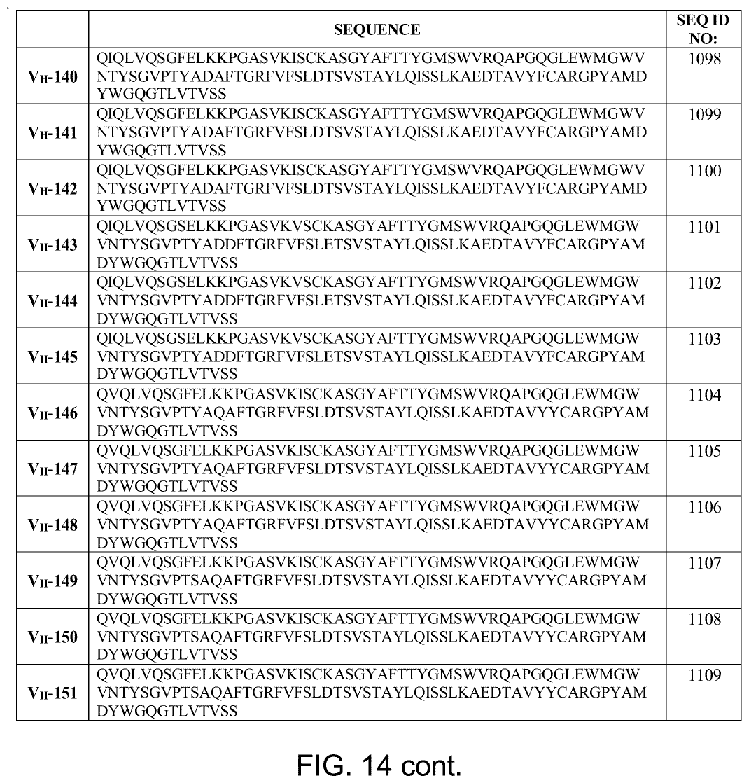

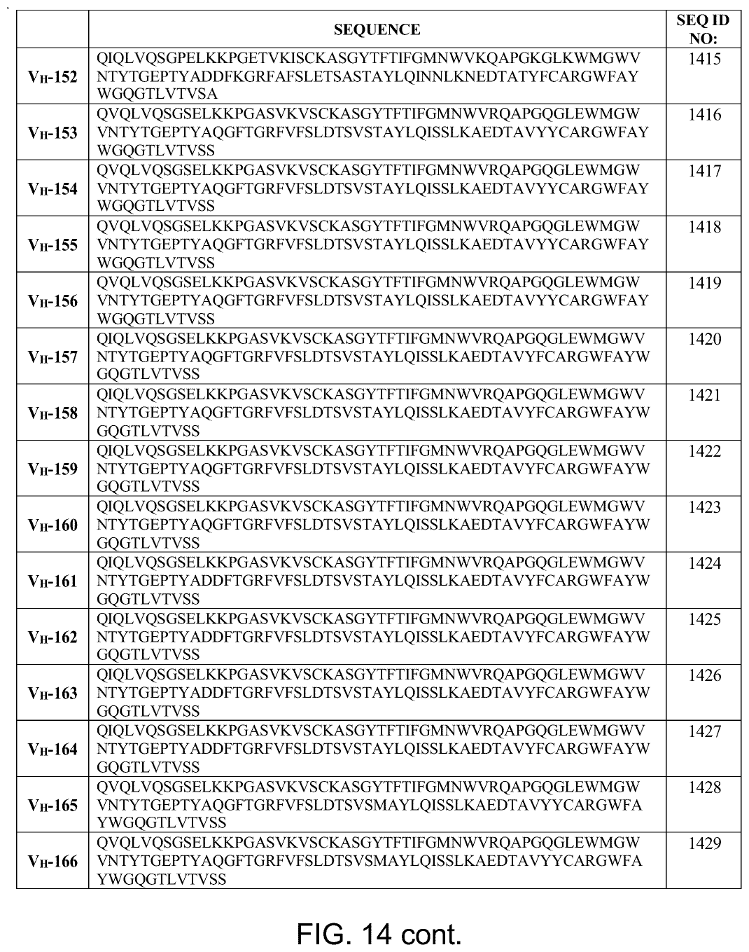

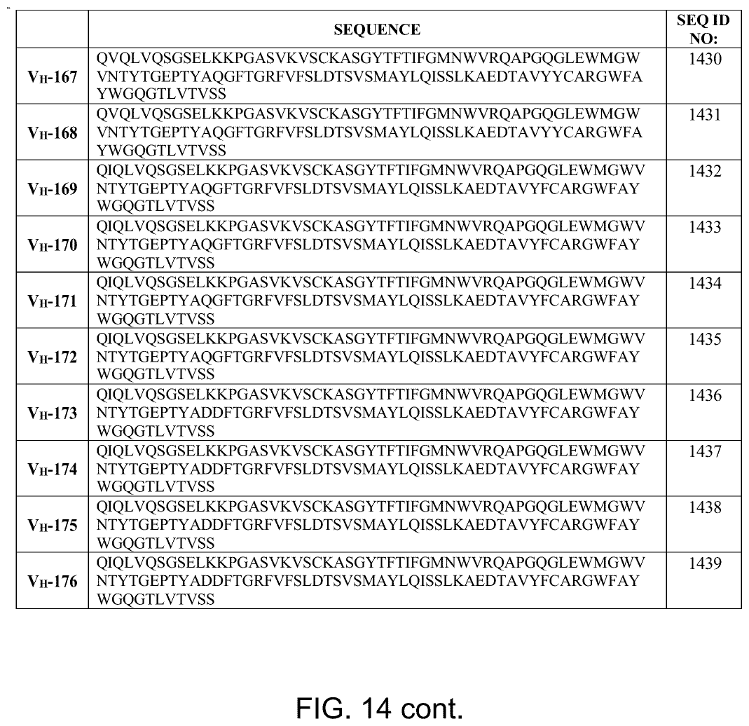

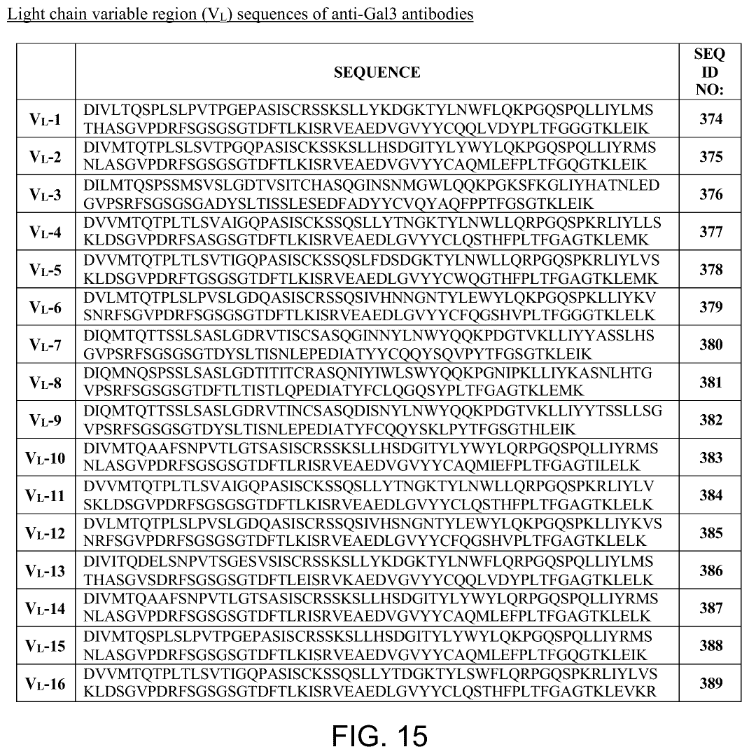

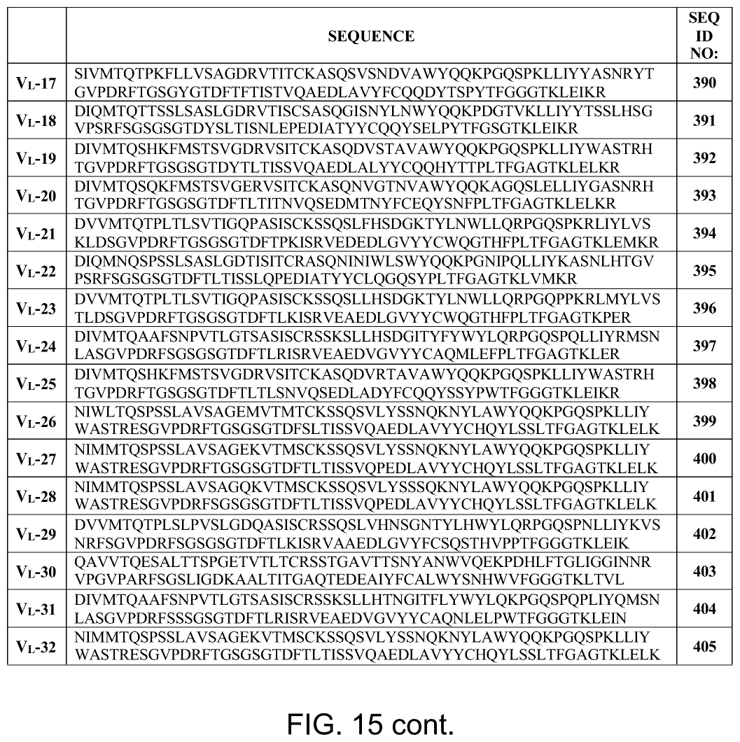

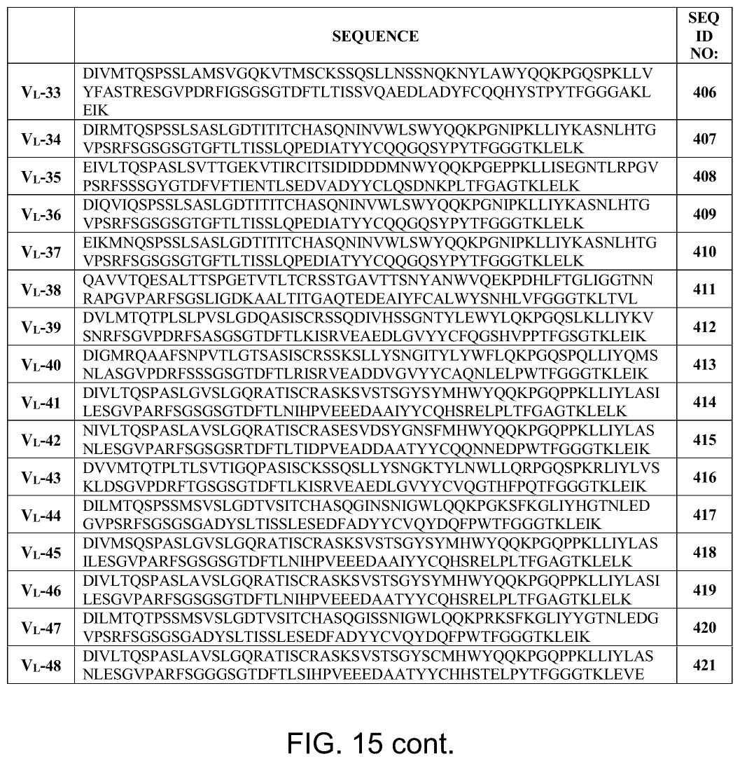

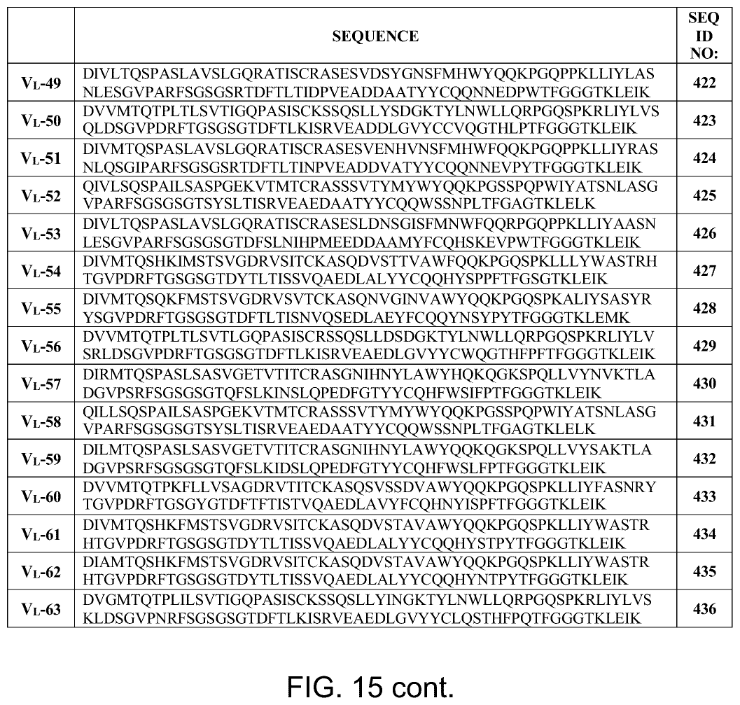

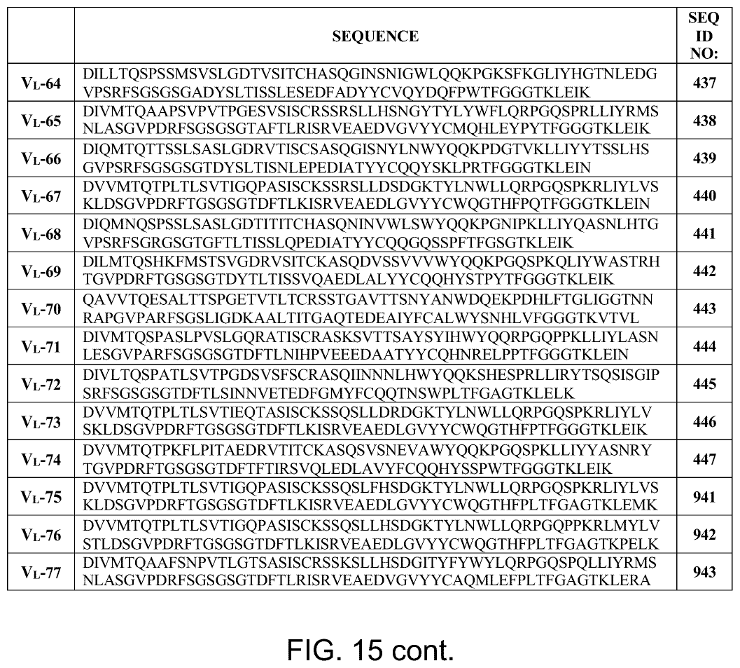

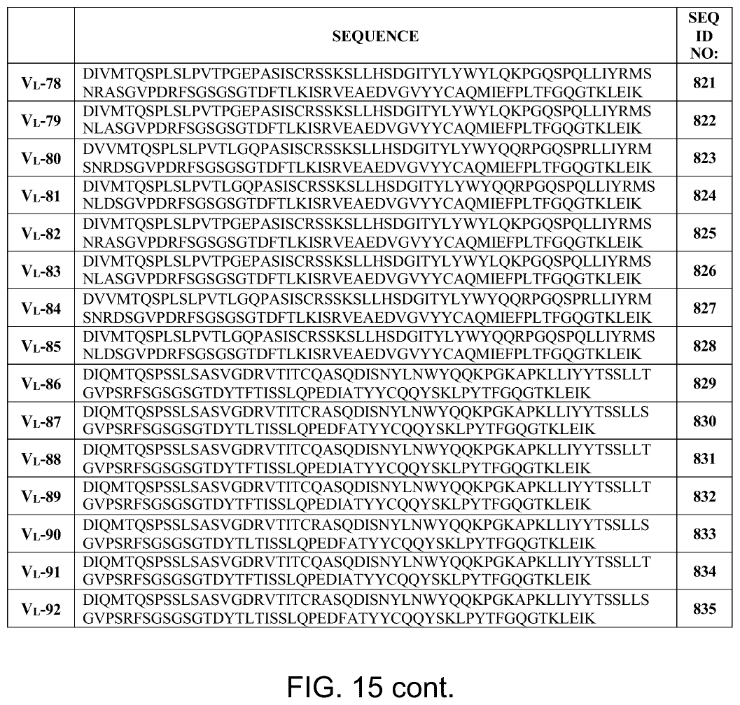

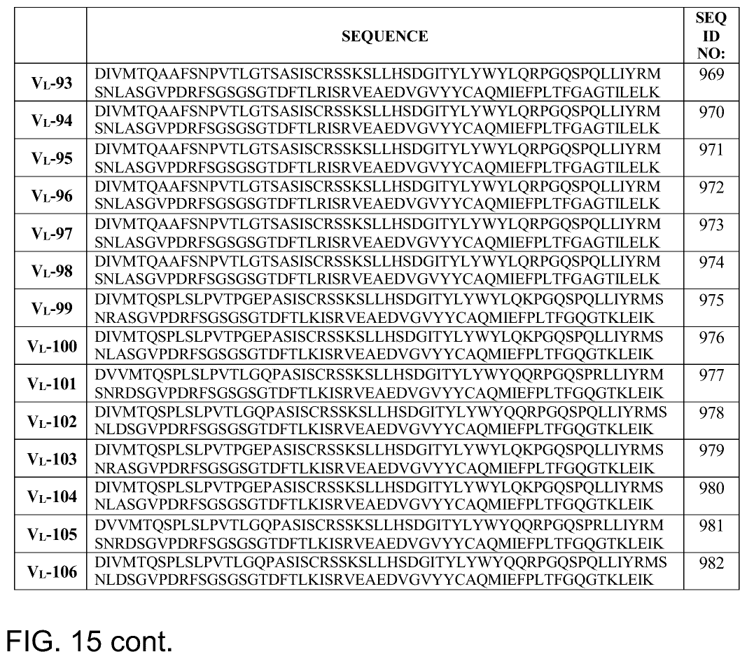

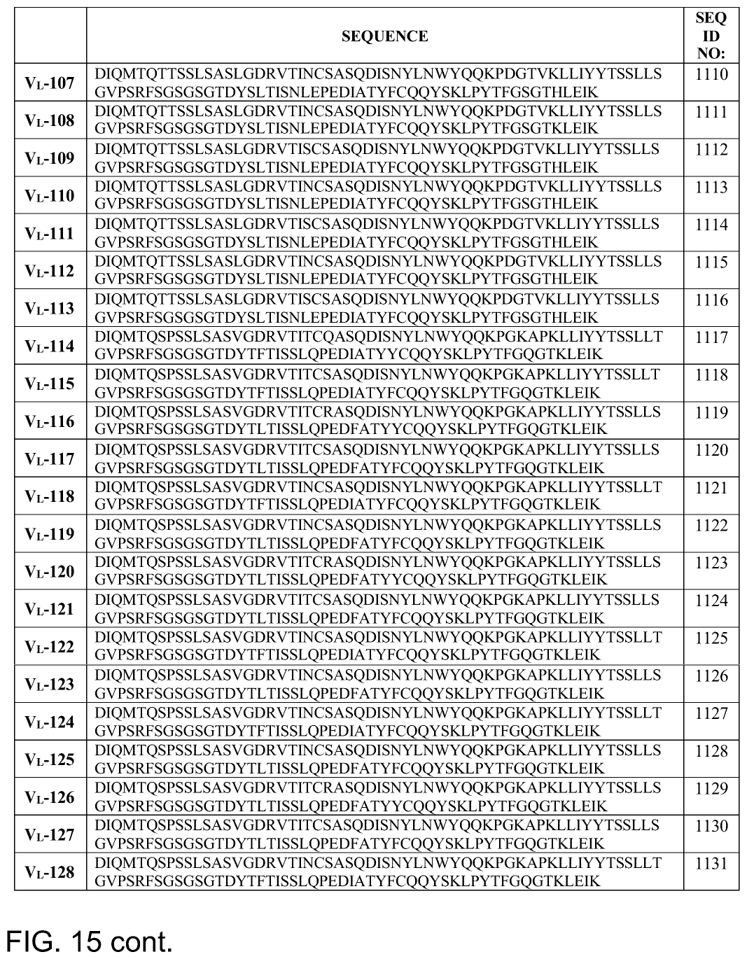

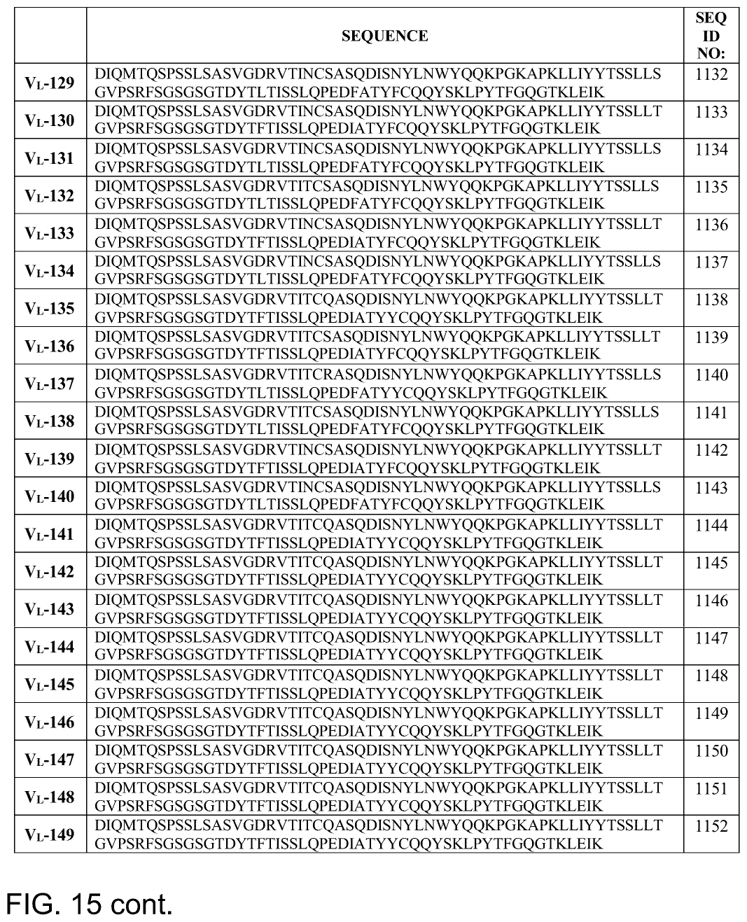

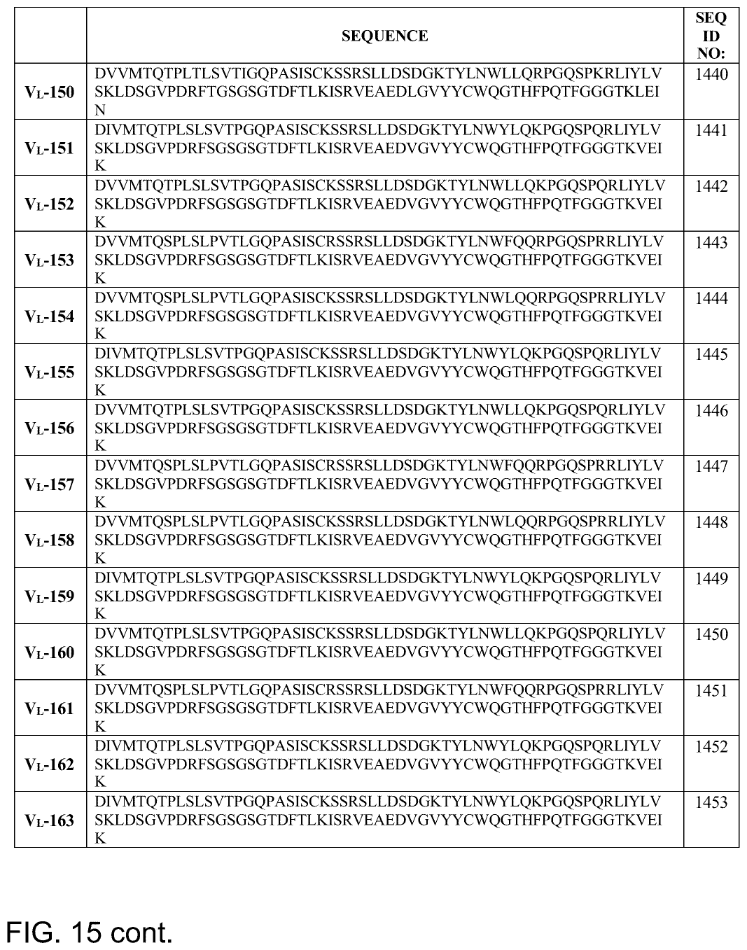

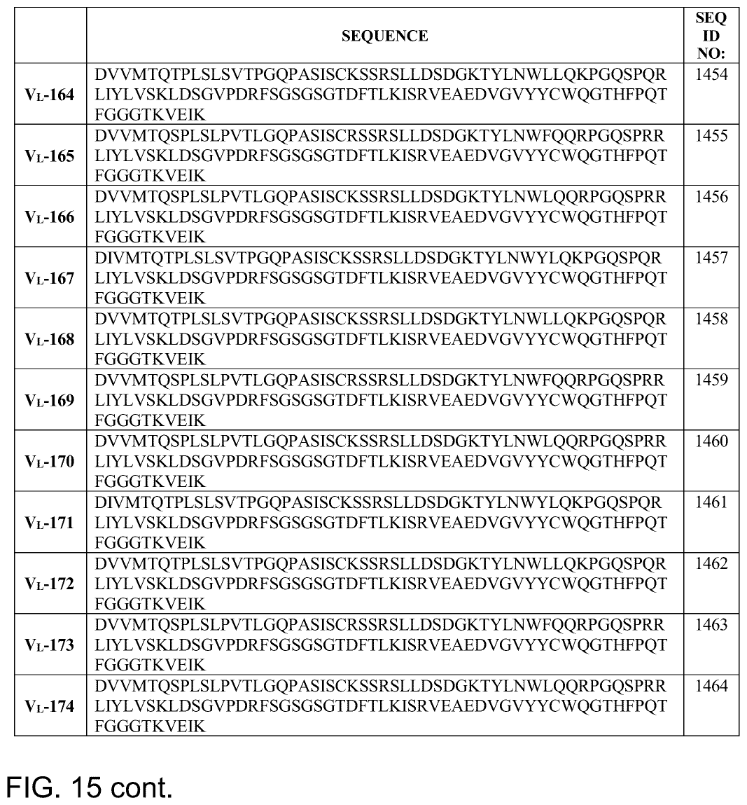

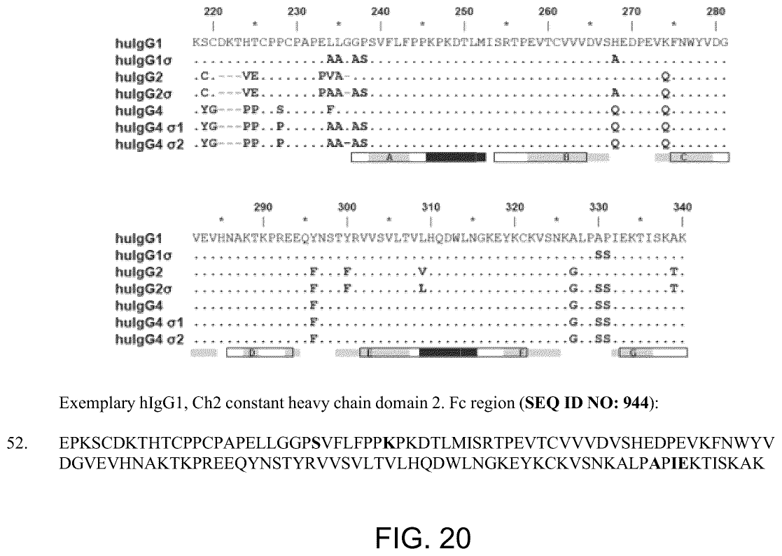

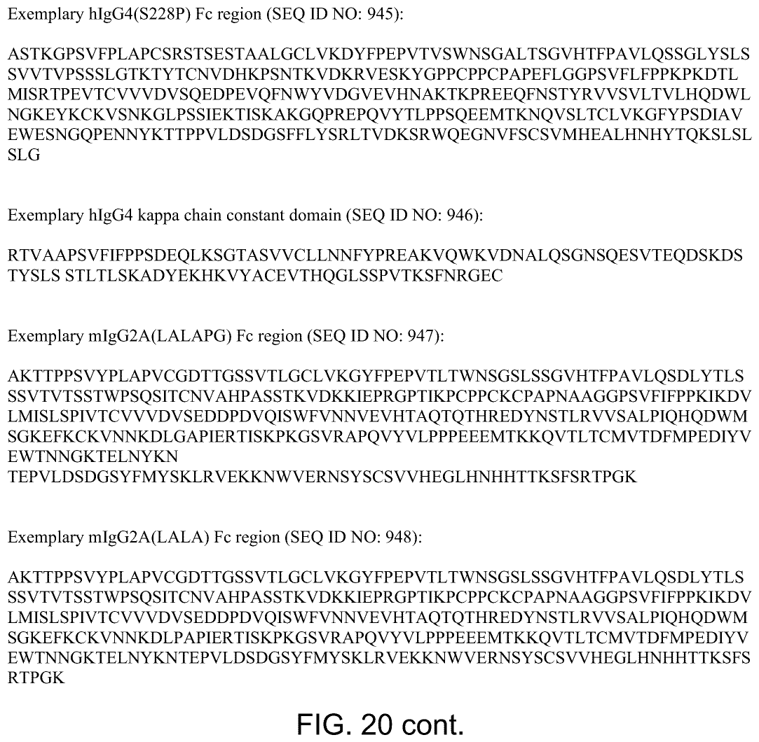

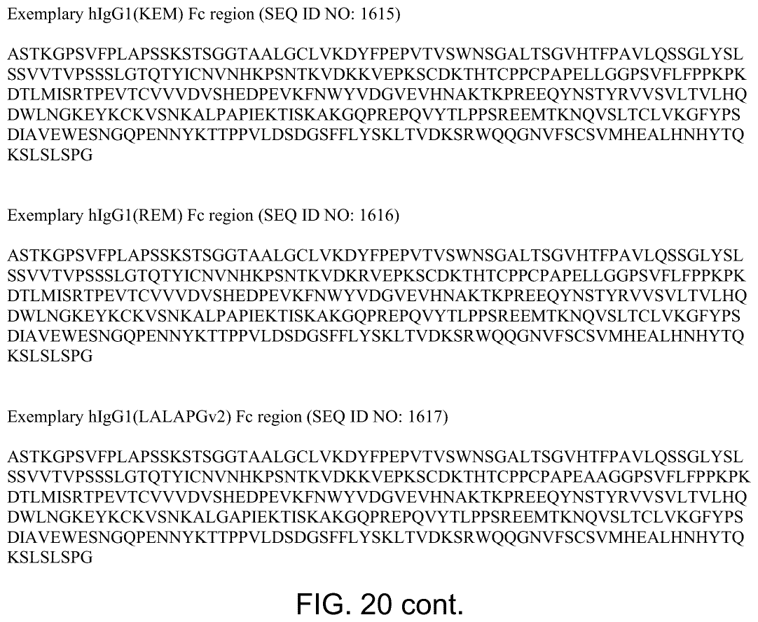

In some embodiments, antibodies or binding fragments thereof are provided. In some embodiments, the antibodies are anti-Gal3 antibodies or binding fragments thereof. In some embodiments, the anti-Gal3 antibodies or binding fragments thereof comprise a heavy chain variable region and a light chain variable region. In some embodiments, the heavy chain variable region is paired with an IgG4 heavy chain constant domain or an IgG2 heavy chain constant domain. In some embodiments, the IgG4 heavy chain constant domain or IgG2 heavy chain constant domain are human or murine. In some embodiments, the IgG4 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 931. In some embodiments, the IgG4 heavy chain constant domain is an S228P mutant. In some embodiments, the IgG2 heavy chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 933 or SEQ ID NO: 934. In some embodiments, the IgG2 heavy chain constant domain is a LALAPG or a LALA mutant. In some embodiments, the light chain variable region is paired with an IgG4 kappa chain constant domain. In some embodiments, the IgG4 kappa chain constant domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 932. Exemplary heavy chain and light chain constant domains can be found in FIG. 20. In some embodiments, the light chain variable region and/or heavy chain variable region may be selected from those depicted in FIGS. 14 and 15 and/or the combinations of light chain variable region and heavy chain variable region as depicted in FIG. 20. In some embodiments, the light chain variable region and/or heavy chain variable regions comprise one or more CDRs depicted in FIG. 12A-C, 13A-C and/or the combinations of CDRs depicted in FIG. 16. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the antibody or binding fragment thereof comprises a sequence (e.g. CDR, VL, VH, LC, HC) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to a sequence of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof binds to specific epitopes within a Gal3 protein. In some embodiments, the anti-Gal3 antibody or binding fragment thereof binds to a specific epitope within a Gal3 protein having an amino acid sequence according to SEQ ID NO: 1-2, provided in FIG. 10. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within a peptide illustrated in FIG. 11 (SEQ ID NOs: 3-26). In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 1-20 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 31-50 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 51-70 of SEQ ID NO: 1-2. In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within amino acid residues 61-80 of SEQ ID NO: 1-2. In some embodiments, any antibody (human, humanized, or not) that competes for binding to any one or more of the proceeding antibodies (optionally at, at least 80% competition as defined in the present examples), can be used in the method as well. In some embodiments, the antibody binds to the same or overlapping epitope of any one or more of the preceding antibodies.

In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3), Peptide 4 (SEQ ID NO: 6), Peptide 6 (SEQ ID NO: 8), or Peptide 7 (SEQ ID NO: 9). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 4 (SEQ ID NO: 6). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 6 (SEQ ID NO: 8). In some embodiments, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 7 (SEQ ID NO: 9). In some embodiments, the anti-Gal3 antibody or binding fragment thereof binds to an epitope present within a region of Gal3 defined by Peptide 1 (SEQ ID NO: 3). In some embodiments, the anti-Gal3 antibody or binding fragment thereof binds to an epitope present within a region of Gal3 defined by Peptide 4 (SEQ ID NO: 6). In some embodiments, the anti-Gal3 antibody or binding fragment thereof binds to an epitope present within a region of Gal3 defined by Peptide 6 (SEQ ID NO: 8). In some embodiments, the anti-Gal3 antibody or binding fragment thereof binds to an epitope present within a region of Gal3 defined by Peptide 7 (SEQ ID NO: 9). In some embodiments, the antibody is one that binds to 1, 2, or all 3 of peptides 1, 6, and/or 7. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, an anti-Gal3 antibody or binding fragment thereof as described herein may bind to the N-terminal domain of Gal3 or a portion thereof. In some embodiments, an anti-Gal3 antibody or binding fragment thereof as described herein may bind to an epitope of Gal3 that includes a motif of GxYPG, where x is the amino acids alanine (A), glycine (G), or valine (V). In some embodiments, an anti-Gal3 antibody or binding fragment thereof as described herein may bind to an epitope of Gal3 that includes two GxYPG motifs separated by three amino acids, where x is A, G, or V.

In some embodiments, the anti-Gal3 antibody or binding fragment thereof binds to Gal3. In some embodiments, the anti-Gal3 antibody or binding fragment thereof binds to the N-terminus of Gal3, the N-terminal domain of Gal3, or the TRD of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3. In some embodiments, the anti-Gal3 antibody or binding fragment thereof binds to Gal3 isoform 1. In some embodiments, the anti-Gal3 antibody or binding fragment thereof binds to the N-terminus of Gal3 isoform 1, the N-terminal domain of Gal3 isoform 1, amino acids 1-111 of Gal3 isoform 1, the TRD of Gal3 isoform 1, or amino acids 36-109 of Gal3 isoform 1. In some embodiments, the anti-Gal3 antibody or binding fragment thereof does not bind to the N-terminus of Gal3 isoform 1, the N-terminal domain of Gal3 isoform 1, amino acids 1-111 of Gal3, the TRD of Gal3 isoform 1, or amino acids 36-109 of Gal3 isoform 1. In some embodiments, the anti-Gal3 antibody or binding fragment thereof binds to the C-terminus of Gal3 isoform 1, the C-terminal domain of Gal3 isoform 1, amino acids 112-250 of Gal3, or the CRD of Gal3. In some embodiments, the anti-Gal3 antibody or binding fragment thereof does not bind to the C-terminus of Gal3 isoform 1, the C-terminal domain of Gal3 isoform 1, amino acids 112-250 of Gal3 isoform 1, or the CRD of Gal3. In some embodiments, the anti-Gal3 antibody or binding fragment thereof binds to the N-terminus of Gal3 isoform 3, the N-terminal domain of Gal3 isoform 3, amino acids 1-125 of Gal3, the TRD of Gal3 isoform 3, or amino acids 50-123 of Gal3 isoform 3. In some embodiments, the anti-Gal3 antibody or binding fragment thereof does not bind to the N-terminus of Gal3 isoform 3, the N-terminal domain of Gal3 isoform 3, amino acids 1-125 of Gal3 isoform 3, the TRD of Gal3, or amino acids 50-123 of Gal3 isoform 3. In some embodiments, the anti-Gal3 antibody or binding fragment thereof binds to the C-terminus of Gal3 isoform 3, the C-terminal domain of Gal3 isoform 3, amino acids 126-264 of Gal3 isoform 3, or the CRD of Gal3. In some embodiments, the anti-Gal3 antibody or binding fragment thereof does not bind to the C-terminus of Gal3 isoform 3, the C-terminal domain of Gal3 isoform 3, amino acids 126-264 of Gal3 isoform 3, or the CRD of Gal3 isoform 3.

In some embodiments, the interaction between Gal3 and a cell surface marker can be reduced to less than 80%, less than 75%, less than 70%, less than 60%, less than 59%, less than 50%, less than 40%, less than 34%, less than 30%, less than 20%, less than 14%, less than 10%, less than 7%, less than 5%, less than 4%, or less than 1%.

In some embodiments, the anti-Gal3 antibody or binding fragment thereof binds to Gal3 with a dissociation constant (K_{D}) of less than 1 nM, less than 1.2 nM, less than 2 nM, less than 5 nM, less than 10 nM, less than 13.5 nM, less than 15 nM, less than 20 nM, less than 25 nM, or less than 30 nM. In some embodiments, the anti-Gal3 antibody or binding fragment thereof binds to Gal3 with a K_{D} of less than 1 nM. In some embodiments, the anti-Gal3 antibody or binding fragment thereof binds to Gal3 with a K_{D} of less than 1.2 nM. In some embodiments, the anti-Gal3 antibody or binding fragment thereof binds to Gal3 with a K_{D} of less than 2 nM. In some embodiments, the anti-Gal3 antibody or binding fragment thereof binds to Gal3 with a K_{D} of less than 5 nM. In some embodiments, the anti-Gal3 antibody or binding fragment thereof binds to Gal3 with a K_{D} of less than 10 nM. In some embodiments, the anti-Gal3 antibody or binding fragment thereof binds to Gal3 with a K_{D} of less than 13.5 nM. In some embodiments, the anti-Gal3 antibody or binding fragment thereof binds to Gal3 with a K_{D} of less than 15 nM. In some embodiments, the anti-Gal3 antibody or binding fragment thereof binds to Gal3 with a K_{D} of less than 20 nM. In some embodiments, the anti-Gal3 antibody or binding fragment thereof binds to Gal3 with a K_{D} of less than 25 nM. In some embodiments, the anti-Gal3 antibody or binding fragment thereof binds to Gal3 with a K_{D} of less than 30 nM.

In some embodiments, the anti-Gal3 antibody or binding fragment thereof comprises any one of the variable heavy chain complementarity-determining region 1 (V_{H}-CDR1) sequences illustrated in FIG. 12A (SEQ ID NOs: 27-70). In some embodiments, the anti-Gal3 antibody comprises a V_{H}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 27-70. In some embodiments, the anti-Gal3 antibody comprises a V_{H}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 27-70. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises any one of the variable heavy chain complementarity-determining region 2 (V_{H}-CDR2) sequences illustrated in FIG. 12B (SEQ ID NOs: 71-111, 801, 951, 952). In some embodiments, the anti-Gal3 antibody or binding fragment thereof comprises a V_{H}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 71-111, 801, 951, 952. In some embodiments, the anti-Gal3 antibody or binding fragment thereof comprises a V_{H}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 71-111, 801, 951, 952. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises any one of the variable heavy chain complementarity-determining region 3 (V_{H}-CDR3) sequences illustrated in FIG. 12C (SEQ ID NOs: 112-169, 802, 953, 954). In some embodiments, the anti-Gal3 antibody or binding fragment thereof comprises a V_{H}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 112-169, 802, 953, 954. In some embodiments, the anti-Gal3 antibody or binding fragment thereof comprises a V_{H}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 112-169, 802, 953, 954. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises any one of the variable light chain complementarity-determining region 1 (V_{L}-CDR1) sequences illustrated in FIG. 13A (SEQ ID NOs: 170-220). In some embodiments, the anti-Gal3 antibody or binding fragment thereof comprises a V_{L}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 170-220. In some embodiments, the anti-Gal3 antibody or binding fragment thereof comprises a V_{L}-CDR1 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 170-220. In some embodiments, any antibody (human, humanized, or not) that competes for binding to any one or more of the proceeding antibodies (optionally at, at least 80% competition as defined in the present examples), can be used in the method as well. In some embodiments, the antibody binds to the same or overlapping epitope of any one or more of the preceding antibodies.

In some embodiments, the anti-Gal3 antibody or binding fragment thereof comprises any one of the variable light chain complementarity-determining region 2 (V_{L}-CDR2) sequences illustrated in FIG. 13B (SEQ ID NOs: 221-247). In some embodiments, the anti-Gal3 antibody or binding fragment thereof comprises a V_{L}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 221-247. In some embodiments, the anti-Gal3 antibody or binding fragment thereof comprises a V_{L}-CDR2 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 221-247. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises any one of the variable light chain complementarity-determining region 3 (V_{L}-CDR3) sequences illustrated in FIG. 13C (SEQ ID NOs: 248-296). In some embodiments, the anti-Gal3 antibody or binding fragment thereof comprises a V_{L}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 248-296. In some embodiments, the anti-Gal3 antibody or binding fragment thereof comprises a V_{L}-CDR3 sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 248-296. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises a heavy chain variable region (V_{H}) and a light chain variable region (V_{L}). In some embodiments, the V_{H} may comprise a V_{H}-CDR1, a V_{H}-CDR2, and/or a V_{H}-CDR3 selected from any of FIG. 12A-C. In some embodiments, the V_{L} may comprise a V_{L}-CDR1, a V_{L}-CDR2, and/or a V_{L}-CDR3 selected from any of FIG. 13A-C. In some embodiments, the anti-Gal3 antibody or binding fragment thereof comprises CDRs within the V_{H} and V_{L} sequences as illustrated in FIGS. 14 and 15. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises a heavy chain variable region (V_{H}) sequence selected from FIG. 14 (SEQ ID NOs: 297-373, 803, 806-820, 955-968, 1067-1109, 1415-1439). In some embodiments, the anti-Gal3 antibody or binding fragment thereof comprises a V_{H}-sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 297-373, 803, 806-820, 955-968, 1067-1109, 1415-1439. In some embodiments, the anti-Gal3 antibody or binding fragment thereof comprises a V_{H}-sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 297-373, 803, 806-820, 955-968, 1067-1109, 1415-1439. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises a light chain variable region (V_{L}) sequence selected from FIG. 12 (SEQ ID NOs: 374-447, 821-835, 969-982, 1110-1152, 1440-1464). In some embodiments, the anti-Gal3 antibody or binding fragment thereof comprises a V_{L} sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 374-447, 821-835, 969-982, 1110-1152, 1440-1464. In some embodiments, the anti-Gal3 antibody or binding fragment thereof comprises a V_{L} sequence having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to any one of SEQ ID NOs: 374-447, 821-835, 969-982, 1110-1152, 1440-1464. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

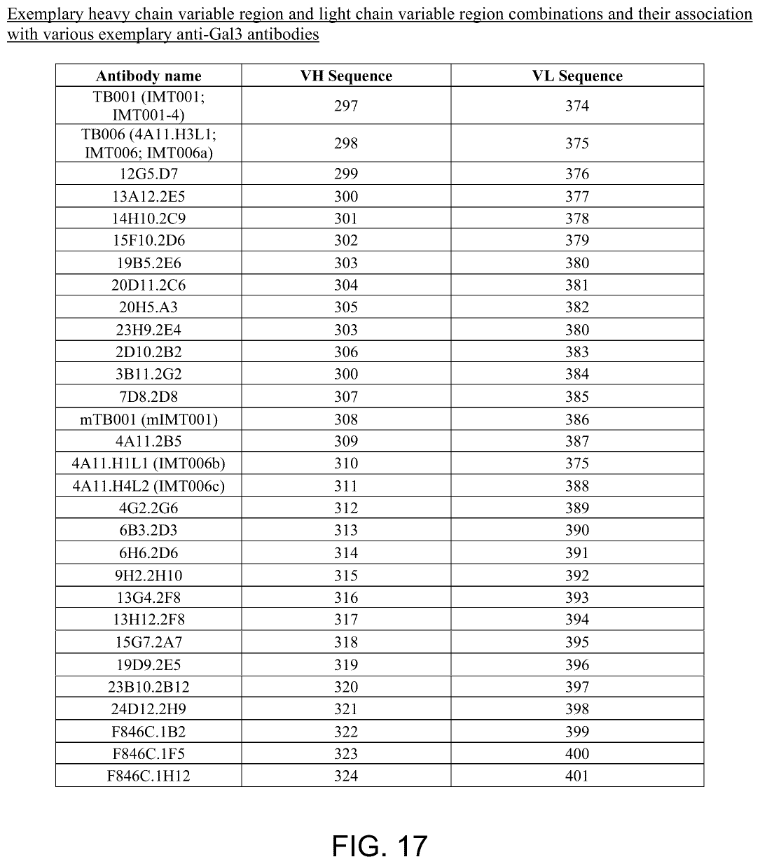

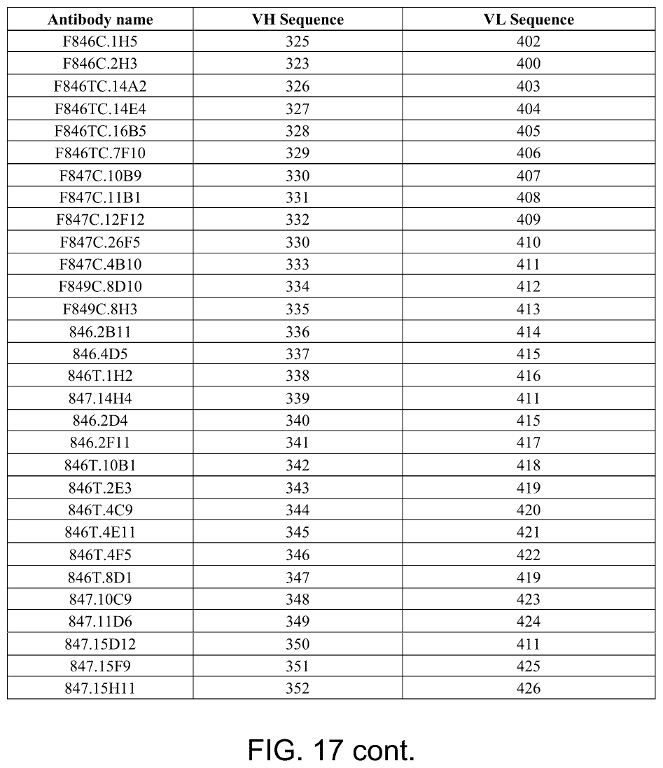

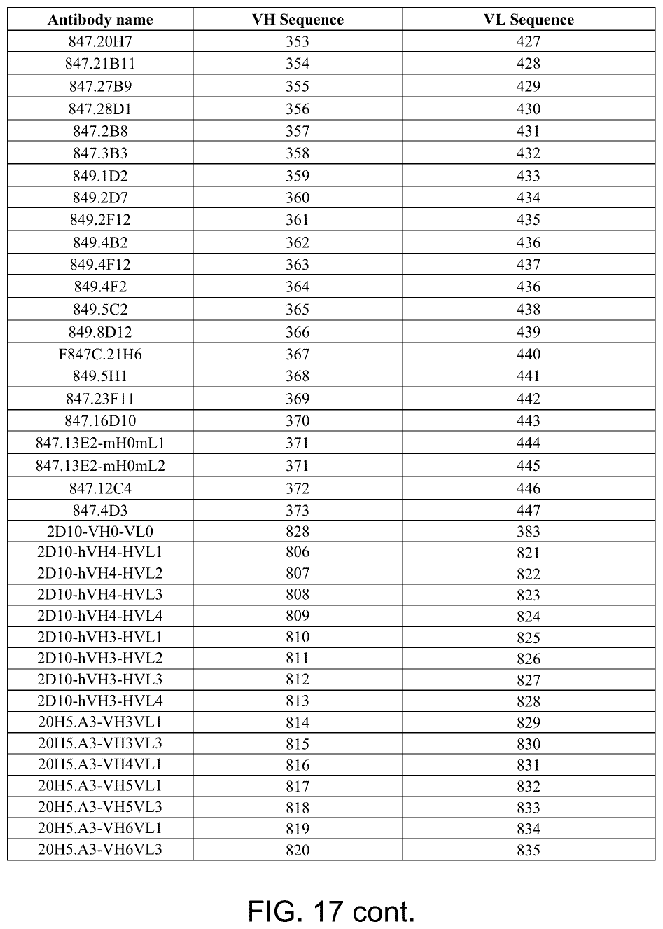

In some embodiments, the anti-Gal3 antibody or binding fragment thereof comprises a combination of heavy chain variable region and light chain variable region as illustrated in FIG. 17.

In some embodiments, the anti-Ga13 antibody or binding fragment thereof comprises heavy chain and light chain sequences as illustrated in FIG. 18 (SEQ ID NOs: 448-538, 804-805, 836-865. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof is selected from the group of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof. In some embodiments, the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises one or more heavy chain variable region CDRs depicted in FIG. 12A-C. In some embodiments, the anti-Gal3 antibody or binding fragment thereof comprises one or more light chain variable region CDRs depicted in FIG. 13A-C. In some embodiments, the anti-Gal3 antibody or binding fragment thereof comprises a heavy chain variable region depicted in FIG. 14. In some embodiments, the anti-Gal3 antibody or binding fragment thereof comprises a light chain variable region depicted in FIG. 15. In some embodiments, the anti-Gal3 antibody or binding fragment thereof comprises a combination of heavy chain variable region and light chain variable region depicted in FIG. 17. In some embodiments, the anti-Gal3 antibody or binding fragment thereof comprises a heavy chain and/or light chain depicted in FIG. 18. In some embodiments, the anti-Gal3 antibody or binding fragment thereof can comprise or include any one or more of the sequences provided in any one or more of FIG. 12A-C, 13A-C, 14, 15, 16, 17, 18, or any one or more of a sequence that is at least 80%, 81%, 82%, 83%, 84%, 85%, 86%, 87%, 88%, 89%, 90%, 91%, 92%, 93%, 94%, 95%, 96%, 97%, 98%, 99% or greater identical thereto. In some embodiments, the anti-Gal3 antibody or binding fragment thereof can comprise or include any one or more of the sequences provided in any one or more of FIG. 12-A-C, 13A-C, 14, 15, 16, 17, 18, or any one or more of a sequence that is at least 80%, 81%, 82%, 83%, 84%, 85%, 86%, 87%, 88%, 89%, 90%, 91%, 92%, 93%, 94%, 95%, 96%, 97%, 98%, 99% or greater similar thereto. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the anti-Gal3 antibody or binding fragment thereof comprises a humanized antibody or binding fragment thereof. In other instances, the anti-Gal3 antibody or binding fragment thereof comprises a chimeric antibody or binding fragment thereof. In some embodiments, the anti-Gal3 antibody comprises a full-length antibody or a binding fragment thereof. In some embodiments, the anti-Gal3 antibody or binding fragment thereof comprises a bispecific antibody or a binding fragment thereof. In some embodiments, the anti-Gal3 antibody or binding fragment thereof comprises a monovalent Fab′, a divalent Fab2, a single-chain variable fragment (scFv), a diabody, a minibody, a nanobody, a single-domain antibody (sdAb), or a camelid antibody or binding fragment thereof.

In some embodiments, the anti-Gal3 antibody or binding fragment thereof is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Biclonics, Fab-scFv-Fc, Two-in-one/Dual Action Fab (DAF), FinomAb, scFv-Fc-(Fab)-fusion, Dock-aNd-Lock (DNL), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some embodiments, the anti-Gal3 antibody or binding fragment thereof is a bispecific antibody or binding fragment thereof comprising a bispecific antibody format illustrated in Brinkmann and Kontermann, “The making of bispecific antibodies,” MABS 9(2): 182-212 (2017).

In some embodiments, the anti-Gal3 antibody or binding fragment thereof can comprise an IgM, IgG (e.g., IgG1, IgG2, IgG3, or IgG4), IgA, or IgE framework. The IgG framework can be IgG1, IgG2, IgG3 or IgG4. In some embodiments, the anti-Gal3 antibody or binding fragment thereof comprises an IgG1 framework. In some embodiments, the anti-Gal3 antibody or binding fragment thereof comprises an IgG2 framework. In some embodiments, the anti-Gal3 antibody or binding fragment thereof comprises an IgG4 framework. The anti-Gal3 antibody or binding fragment thereof can further comprise a Fc mutation.

In some embodiments, the Fc region comprises one or more mutations that modulate Fc receptor interactions, e.g., to enhance effector functions such as ADCC and/or CDC. In such instances, exemplary residues when mutated modulate effector functions include S239, K326, A330, 1332, or E333, in which the residue position correspond to IgG1 and the residue numbering is in accordance to Kabat numbering (EU index of Kabat et al 1991 Sequences of Proteins of Immunological Interest). In some embodiments, the one or more mutations comprise S239D, K326W, A330L, 1332E, E333A, E333S, or a combination thereof. In some embodiments, the one or more mutations comprise S239D, 1332E, or a combination thereof. In some embodiments, the one or more mutations comprise S239D, A330L, 1332E, or a combination thereof. In some embodiments, the one or more mutations comprise K326W, E333S, or a combination thereof. In some embodiments, the mutation comprises E333A.

In some embodiments, an anti-Gal3 antibody or binding fragment thereof comprises a humanization score of above 70, above 80, above 81, above 82, above 83, above 84, above 85, above 86, above 87, above 88, above 89, above 90, or above 95. In some embodiments, the anti-Gal3 antibody or binding fragment thereof comprises a humanization score of above 80. In some embodiments, the anti-Gal3 antibody or binding fragment thereof comprises a humanization score of above 83. In some embodiments, the anti-Gal3 antibody or binding fragment thereof comprises a humanization score of above 85. In some embodiments, the anti-Gal3 antibody or binding fragment thereof comprises a humanization score of above 87. In some embodiments, the anti-Gal3 antibody or binding fragment thereof comprises a humanization score of above 90. In some embodiments, the anti-Gal3 antibody or binding fragment thereof comprises a humanization score of the heavy chain of above 70, above 80, above 81, above 82, above 83, above 84, above 85, above 86, above 87, above 88, above 89, above 90, or above 95, optionally above 80, above 85, or above 87. In some embodiments, the anti-Gal3 antibody or binding fragment thereof comprises a humanization score of the light chain of above 70, above 80, above 81, above 82, above 83, above 84, above 85, above 86, above 87, above 88, above 89, above 90, or above 95, optionally above 80, above 83, or above 85.

Also disclosed herein are proteins. In some embodiments, the proteins comprise one or more of SEQ ID NOs: 27-538, 801-865, 955-1010, 1067-1238, 1415-1514. In some embodiments, the proteins comprise a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of SEQ ID NOs: 27-538, 801-865, 955-1010, 1067-1238, 1415-1514. In some embodiments, the proteins comprise a sequence having at least 0, 1, 2, 3, 4, 5, or 6 substitutions relative to any one or more sequences of SEQ ID NOs: 27-538, 801-865, 955-1010, 1067-1238, 1415-1514. In some embodiments, the proteins comprise six sequences selected from each of SEQ ID NOs: 27-70; SEQ ID NOs: 71-111, 801, 951, 952; SEQ ID NOs: 112-169, 802, 953, 954; SEQ ID NOs: 170-220; SEQ ID NOs: 211-247; SEQ ID NOs: 248-296. In some embodiments, the proteins comprise two sequences selected from each of SEQ ID NOs: 297-373, 803, 806-820, 940, 955-968, 1067-1109, 1415-1439, and SEQ ID NOs: 374-447, 821-835, 941-943, 969-982, 1110-1152, 1440-1464. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the proteins comprise two sequences selected from each of SEQ ID NOs: 448-494, 804, 836-850 and SEQ ID NOs: 495-538, 805, 851-865, 997-1010, 1196-1238, 1412, 1490-1514. In some embodiments, the proteins comprise any one or more of the sequences depicted in FIG. 12A-C, 13A-C, 14, 15, 16, 17, 18. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, the protein comprises one or more sequences defined by a consensus sequence. The consensus sequences provided herein have been derived from the alignments of CDRs depicted in FIG. 25A-B. However, it is envisioned that alternative alignments may be done (e.g. using global or local alignment, or with different algorithms, such as Hidden Markov Models, seeded guide trees, Needleman-Wunsch algorithm, or Smith-Waterman algorithm) and as such, alternative consensus sequences can be derived.

In some embodiments, the protein comprises a sequence defined by the formula X₁X₂X₃X₄X₅X₆X₇X₈X₉X₁₀X₁₁X₁₂X₁₃X₁₄X₁₅X₁₆X₁₇, where X₁ is no amino acid or R; X₂ is no amino acid or S; X₃ is no amino acid, S, or T; X₄ is no amino acid, E, G, K, Q, or R; X₅ is no amino acid, A, D, G, I, N, or S; X₆ is no amino acid, I, L, or V; X₇ is no amino acid, F, L, S, or V; X₈ is no amino acid, D, E, H, N, S, T, or Y; X₉ is no amino acid, D, E, I, K, N, R, S, T, or V; X₁₀ is no amino acid, D, H, N, R, S, or Y; X₁₁ is no amino acid, A, G, N, S, T, or V; X₁₂ is no amino acid, A, I, K, N, Q, T, V, or Y; X₁₃ is no amino acid, D, G, H, K, N, S, T, or Y; X₁₄ is no amino acid, C, F, I, N, S, T, V, or Y; X₁₅ is no amino acid, D, L, N, W, or Y; X₁₆ is no amino acid, N, or D; X₁₇ is no amino acid or D. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈, where X₁ is no amino acid, K, L, N, Q, or R; X₂ is no amino acid, A, L, M, or V; X₃ is no amino acid, C, K, or S; X₄ is no amino acid or T; X₅ is no amino acid, A, E, F, G, H, K, Q, R, S, W, or Y; X₆ is no amino acid, A, G, or T; X₇ is no amino acid, I, K, N, S, or T; X₈ is no amino acid, N, or S. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀, where X₁ is no amino acid, A, E, F, H, L, M, Q, S, V, or W; X₂ is A, H, or Q; X₃ is D, F, G, H, L, M, N, Q, S, T, W, or Y; X₄ is no amino acid or W; X₅ is A, D, I, K, L, N, Q, R, S, T, V, or Y; X₆ is D, E, H, I, K, L, N, Q, S, or T; X₇ is D, F, K, L, N, P, S, T, V, W, or Y; X₈ is H, P, or S; X₉ is F, L, P, Q, R, T, W, or Y; X₁₀ is no amino acid, T, or V. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀, where X₁ is E, G, or R; X₂ is F, N, or Y; X₃ is A, I, K, N, S, or T; X₄ is F, I, or L; X₅ is I, K, N, R, S, or T; X₆ is D, G, I, N, S, or T; X₇ is F, G, H, S, or Y; X₈ is no amino acid, A, D, G, I, M, N, T, V, W, or Y; X₉ is no amino acid, M, or Y; X₁₀ is no amino acid or G; In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀, where X₁ is no amino acid, I, or L; X₂ is no amino acid or R; X₃ is no amino acid, F, I, L, or V; X₄ is A, D, F, H, K, L, N, S, W, or Y; X₅ is A, D, P, S, T, W, or Y; X₆ is D, E, G, H, K, N, S, V, or Y; X₇ is D, E, G, N, S, or T; X₈ is D, G, I, K, N, Q, R, S, V, or Y; X₉ is A, D, E, G, I, K, N, P, S, T, V, or Y; X₁₀ is no amino acid, I, P, S, or T. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence.

In some embodiments, the protein comprises a sequence defined by the formula X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅, where X₁ is no amino acid or A; X₂ is no amino acid, A, R, or Y; X₃ is no amino acid, A, F, H, K, L, R, S, or V; X₄ is no amino acid, A, D, K, N, R, S, or T; X₅ is no amino acid, A, D, G, H, I, L, N, P, R, S, T, V, or Y; X₆ is no amino acid, A, D, G, H, K, N, P, Q, R, S, or Y; X₇ is no amino acid, D, F, G, H, P, R, S, W, or Y; X₈ is no amino acid, A, D, E, G, I, R, or S; X₉ is no amino acid, A, C, D, E, F, G, I, N, R, S, T, V, or Y; X₁₀ is no amino acid, A, D, M, P, R, S, T, V, or Y; X₁₁ is no amino acid, A, D, E, F, L, T, V, or Y; X₁₂ is no amino acid, A, G, L, M, R, or T; X₁₃ is no amino acid, A, D, E, F, G, R, S, T, or V; X₁₄ is no amino acid, A, D, G, L, P, Q, R, S, T, V, or Y; X₁₅ is no amino acid, A, D, G, N, S, V, W, or Y; X₁₆ is no amino acid, A, D, E, F, L, P, T, V, W, or Y; X₁₇ is no amino acid, F, I, L, M, R, or Y; X₁₅ is no amino acid, A, D, G, N, or T; X₁₉ is no amino acid, F, N, S, T, V, or Y; X₂₀ is no amino acid or L; X₂₁ is no amino acid or A; X₂₂ is no amino acid or W; X₂₃ is no amino acid or F; X₂₄ is no amino acid or A; X₂₅ is no amino acid or Y. In some embodiments, the protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to this consensus sequence. In some embodiments, the protein comprises a sequence having 0, 1, 2, 3, 4, 5, or 6 substitutions from this consensus sequence. In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies.

In some embodiments, any antibody (human, humanized, or not) that competes for binding to any one or more of the proceeding antibodies (optionally at, at least 80% competition as defined in the present examples, e.g., Example 54), can be used in the method as well. In some embodiments, the antibody binds to the same or overlapping epitope of any one or more of the preceding antibodies. In some embodiments, the 80% competition is determined as follows:
A) Ab (such as TB006 for example) is coated onto surface.
B) Enzyme linked antibodies already bound by Gal3 antibodies (those that one wishes to determine if the Ab competes with, for example TB006—the “potentially competing Ab”) are added to the Elisa plate.
C) The ELISA plate is incubated then washed (to remove any unbound Gal3 bound by the potentially competing Ab).
D) The plate is then collected. Where the potentially competing Ab bound to Gal3 still allows binding to the immobilized Ab (E.g., TB006), then the Ab do not compete. Where it does not (or does so less that fully) then there is that amount of competition.

In some embodiments, the 80% competition value can be determined as outlined in Example 54 herein. In some embodiments, the 80% competition value can be determined as follows: Ab like TB006 is diluted and coated onto an ELISA plate. The plate is incubated and, after incubation, the plate is washed a blocking solution is applied and the plate is incubated again. Following incubation, the blocking solution is removed. Any competing antibody to be tested is mixed with Flag-tagged Gal-3 protein in binding solutions and applied to the Elisa. The plate is incubated and then washed. Afterwards, HRP-conjugated anti-FLAG Gal3 antibodies are added to the plate. The plate is incubated then washed. If the anti-gal3 blocking antibody competes for binding with the anti-Gal3 antibody like TB006, then HRP-conjugated anti-FLAG Gal3 will not be detectable following the wash. If the anti-gal3 blocking antibody does not compete for binding with the anti-Gal3 antibody, then HRP-conjugated anti-FLAG Gal3 will be detectable, or will be detectable to a lesser level following the wash. The plate is then developed and read in a plate reader. Data can be analyzed and/or graphed using any appropriate means.

A human or humanized antibody that competes for binding to GAL3 with TB006, wherein the antibody competes at a level of at least 80% competition. In some embodiments, the antibody is not one or any of: F847C.21H6 ((VH: QIQLVQSGPELKKPGETVKISCKASGYTFTIFGMNWVKQAPGKGLKWMGWVNTYT GEPTYADDFKGRFAFSLETSASTAYLQINNLKNEDTATYFCARGWFAYWGQGTLVT VSA (SEQ ID NO: 367); VL: DVVMTQTPLTLSVTIGQPASISCKSSRSLLDSDGKTYLNWLLQRPGQSPKRLIYLVSK LDSGVPDRFTGSGSGTDFTLKISRVEAEDLGVYYCWQGTHFPQTFGGGTKLEIN (SEQ ID NO:440)), F846TC.14A2 (VH: QIQLVQSGPELKKPGETVKISCKASGYTFTNYGMNWVKQAPGKGLKWMGWINTYT GEPTYAGDLKGRFAFSLETSASTAYLQINNLKNEDTATYFCVRYTMDYWGQGTSVT VSS (SEQ ID NO: 326); VL: QAVVTQESALTTSPGETVTLTCRSSTGAVTTSNYANWVQEKPDHLFTGLIGGINNRV PGVPARFSGSLIGDKAALTITGAQTEDEAIYFCALWYSNHWVFGGGTKLTVL (SEQ ID NO:403)), F847C.12F12 (VH: QIQLVQSGPELKKPGETVKISCKASGYTFTNYGMNWVKQAPGKGLKWMGWINTYT GEPTYVDDFKGRFAFSLETSASTAYLRINNLKNEDTATYFCAKFGNYVGAMDYWGQ GTSVTVSS (SEQ ID NO:343); VL: DIQVIQSPSSLSASLGDTITITCHASQNINVWLSWYQQKPGNIPKLLIYKASNLHTGVP SRFSGSGSGTGFTLTISSLQPEDIATYYCQQGQSYPYTFGGGTKLELK (SEQ ID NO:409)), F846T.16B5 (VH: QIQLVQSGPELKKPGETVKISCKASGYTFTNYGMNWVKQAPGKGLKWMGWINTYT GEPAYADDFKGRFAFSLETSASTAYLQINNLKNEDMATYFCARWGGYDGDYYAMD YWGQGTSVTVSS (SEQ ID NO:328); VL: NIMMTQSPSSLAVSAGEKVTMSCKSSQSVLYSSNQKNYLAWYQQKPGQSPKLLIYW ASTRESGVPDRFTGSGSGTDFTLTISSVQAEDLAVYYCHQYLSSLTFGAGTKLELK (SEQ ID NO:405)), F846C.2H3 (VH: QIQLVQSGPELKKPGETVKISCKASGYTFTNYGMNWVKQAPGKGLKWMGWINTYT GEPSYADDFKGRFAFSLETSASTAYLQINNLKNEDMATYFCARWGGYDGDYYAMD YWGQGTSVTVSS (SEQ ID NO:323); VL: NIMMTQSPSSLAVSAGEKVTMSCKSSQSVLYSSNQKNYLAWYQQKPGQSPKLLIYW ASTRESGVPDRFTGSGSGTDFTLTISSVQPEDLAVYYCHQYLSSLTFGAGTKLELK (SEQ ID NO:400)), F846C.1F5 (VH: QIQLVQSGPELKKPGETVKISCKASGYTFTNYGMNWVKQAPGKGLKWMGWINTYT GEPSYADDFKGRFAFSLETSASTAYLQINNLKNEDMATYFCARWGGYDGDYYAMD YWGQGTSVTVSS (SEQ ID NO:323); VL: NIMMTQSPSSLAVSAGEKVTMSCKSSQSVLYSSNQKNYLAWYQQKPGQSPKLLIYW ASTRESGVPDRFTGSGSGTDFTLTISSVQPEDLAVYYCHQYLSSLTFGAGTKLELK (SEQ ID NO: 400)), TB001 (VH: QVQLVQSGSELKKPGASVKVSCKASGYTFTNYGMNWVRQAPGQGLKWMGWINTN TGEPTYVEEFTGRFVFSLETSVSTAYLQISSLKAEDTAVYFCAPYDNFFAYWGQGTT VTVSS (SEQ ID NO: 297); VL: DIVLTQSPLSLPVTPGEPASISCRSSKSLLYKDGKTYLNWFLQKPGQSPQLLIYLMSTH ASGVPDRFSGSGSGTDFTLKISRVEAEDVGVYYCQQLVDYPLTFGGGTKLEIK (SEQ ID NO: 374)), or Synagis-hIgG4 (VH: QVTLRESGPALVKPTQTLTLTCTFSGFSLSTSGMSVGWIRQPPGKALEWLADIWWDD KKDYNPSLKSRLTISKDTSKNQVVLKVTNMDPADTATYYCARSMITNWYFDVWGA GTTVTVSS (SEQ ID NO: 1911); VL: DIQMTQSPSTLSASVGDRVTITCKCQLSVGYMHWYQQKPGKAPKLLIYDTSKLASG VPSRFSGSGSGTEFTLTISSLQPDDFATYYCFQGSGYPFTFGGGTKLEIK (SEQ ID NO: 1912)). In some embodiments, the 80% competition is determined as follows:
A) Ab like TB006 is diluted and coated onto an ELISA plate.
B) The plate is incubated and, after incubation, the plate is washed, a blocking solution is applied and the plate is incubated again
C) The blocking solution is removed.
D) Ab (potentially competing Ab) is mixed with Flag-tagged Gal-3 protein and are added to the Elisa plate.
E) The ELISA plate is incubated then washed.
F) The plate is then developed and read in a plate reader.
G) Data can be analyzed and/or graphed using any appropriate means.

In some embodiments, the 80% competition value can be determined as outlined in Example 54 herein. In some embodiments, the 80% competition value can be determined as follows: Gal3 Ab like TB006 diluted in PBS and coated in a 96-well ELISA plate. The plate is incubated overnight and then washed with three times, followed by a application of a blocking solution and incubation for an hour with gentle rocking. The existing blocking solution is then discarded from the plate. Any competing antibody is mixed with Flag-tagged Gal-3 protein and is then applied to the plate. The plate is incubated for an hour at RT with gentle rocking, then washed with PBST. Afterwards, HRP-tagged anti-FLAG Gal3 antibodies are diluted in PBST and added to the plate. The plate is incubated at RT with gentle rocking, then washed with PBST. If the anti-gal3 blocking antibody competes for binding with the anti-Gal3 antibody, then HRP-tagged anti-FLAG Gal3 will not be detectable following the wash. If the anti-gal3 blocking antibody does not compete for binding with the anti-Gal3 antibody, then HRP-tagged anti-FLAG Gal3 will be detectable, or will be detectable to a lesser level following the wash. To develop the plate, ABTS substrate is added to each well and incubated until a sufficiently high signal is achieved. The plate is read in a plate reader at an absorbance of 405 nm. Data is then analyzed and/or visualized using any appropriate means such as graphing the data using GraphPad Prism 8.0 software (GraphPad Software Inc).

A human or humanized antibody that competes for binding to GAL3 with TB006, wherein the antibody competes at a level of at least 80% competition. In some embodiments, the antibody is not one or any of: F847C.21H6 ((VH: QIQLVQSGPELKKPGETVKISCKASGYTFTIFGMNWVKQAPGKGLKWMGWVNTYT GEPTYADDFKGRFAFSLETSASTAYLQINNLKNEDTATYFCARGWFAYWGQGTLVT VSA (SEQ ID NO: 367); VL: DVVMTQTPLTLSVTIGQPASISCKSSRSLLDSDGKTYLNWLLQRPGQSPKRLIYLVSK LDSGVPDRFTGSGSGTDFTLKISRVEAEDLGVYYCWQGTHFPQTFGGGTKLEIN (SEQ ID NO:440)), F846TC.14A2 (VH: QIQLVQSGPELKKPGETVKISCKASGYTFTNYGMNWVKQAPGKGLKWMGWINTYT GEPTYAGDLKGRFAFSLETSASTAYLQINNLKNEDTATYFCVRYTMDYWGQGTSVT VSS (SEQ ID NO: 326); VL: QAVVTQESALTTSPGETVTLTCRSSTGAVTTSNYANWVQEKPDHLFTGLIGGINNRV PGVPARFSGSLIGDKAALTITGAQTEDEAIYFCALWYSNHWVFGGGTKLTVL (SEQ ID NO:403)), F847C.12F12 (VH: QIQLVQSGPELKKPGETVKISCKASGYTFTNYGMNWVKQAPGKGLKWMGWINTYT GEPTYVDDFKGRFAFSLETSASTAYLRINNLKNEDTATYFCAKFGNYVGAMDYWGQ GTSVTVSS (SEQ ID NO:343); VL: DIQVIQSPSSLSASLGDTITITCHASQNINVWLSWYQQKPGNIPKLLIYKASNLHTGVP SRFSGSGSGTGFTLTISSLQPEDIATYYCQQGQSYPYTFGGGTKLELK (SEQ ID NO:409)), F846T.16B5 (VH: QIQLVQSGPELKKPGETVKISCKASGYTFTNYGMNWVKQAPGKGLKWMGWINTYT GEPAYADDFKGRFAFSLETSASTAYLQINNLKNEDMATYFCARWGGYDGDYYAMD YWGQGTSVTVSS (SEQ ID NO:328); VL: NIMMTQSPSSLAVSAGEKVTMSCKSSQSVLYSSNQKNYLAWYQQKPGQSPKLLIYW ASTRESGVPDRFTGSGSGTDFTLTISSVQAEDLAVYYCHQYLSSLTFGAGTKLELK (SEQ ID NO:405)), F846C.2H3 (VH: QIQLVQSGPELKKPGETVKISCKASGYTFTNYGMNWVKQAPGKGLKWMGWINTYT GEPSYADDFKGRFAFSLETSASTAYLQINNLKNEDMATYFCARWGGYDGDYYAMD YWGQGTSVTVSS (SEQ ID NO:323); VL: NIMMTQSPSSLAVSAGEKVTMSCKSSQSVLYSSNQKNYLAWYQQKPGQSPKLLIYW ASTRESGVPDRFTGSGSGTDFTLTISSVQPEDLAVYYCHQYLSSLTFGAGTKLELK (SEQ ID NO:400)), F846C.1F5 (VH: QIQLVQSGPELKKPGETVKISCKASGYTFTNYGMNWVKQAPGKGLKWMGWINTYT GEPSYADDFKGRFAFSLETSASTAYLQINNLKNEDMATYFCARWGGYDGDYYAMD YWGQGTSVTVSS (SEQ ID NO:323); VL: NIMMTQSPSSLAVSAGEKVTMSCKSSQSVLYSSNQKNYLAWYQQKPGQSPKLLIYW ASTRESGVPDRFTGSGSGTDFTLTISSVQPEDLAVYYCHQYLSSLTFGAGTKLELK (SEQ ID NO: 400)), TB001 (VH: QVQLVQSGSELKKPGASVKVSCKASGYTFTNYGMNWVRQAPGQGLKWMGWINTN TGEPTYVEEFTGRFVFSLETSVSTAYLQISSLKAEDTAVYFCAPYDNFFAYWGQGTT VTVSS (SEQ ID NO: 297); VL: DIVLTQSPLSLPVTPGEPASISCRSSKSLLYKDGKTYLNWFLQKPGQSPQLLIYLMSTH ASGVPDRFSGSGSGTDFTLKISRVEAEDVGVYYCQQLVDYPLTFGGGTKLEIK (SEQ ID NO: 374)), or Synagis-hIgG4 (VH: QVTLRESGPALVKPTQTLTLTCTFSGFSLSTSGMSVGWIRQPPGKALEWLADIWWDD KKDYNPSLKSRLTISKDTSKNQVVLKVTNMDPADTATYYCARSMITNWYFDVWGA GTTVTVSS (SEQ ID NO: 1911); VL: DIQMTQSPSTLSASVGDRVTITCKCQLSVGYMHWYQQKPGKAPKLLIYDTSKLASG VPSRFSGSGSGTEFTLTISSLQPDDFATYYCFQGSGYPFTFGGGTKLEIK (SEQ ID NO: 1912)). In some embodiments, the 80% competition is determined as follows:
A) Gal3 Ab diluted in PBS and coated a 96-well ELISA plate.
B) The plate is incubated overnight and then washed with three times, followed by a application of a blocking solution and incubation for an hour with gentle rocking.
C) The existing blocking solution is then discarded from the ELISA plate.
D) Competing antibody is mixed with Flag-tagged Gal-3 in binding solutions and then applied to the ELISA plate.
E) The ELISA plate is incubated for an hour at RT with gentle rocking, then washed with PBST.
F) HRP-conjugated anti-FLAG Gal3 antibodies are diluted in PBST and added to the plate.

G) The plate is incubated at RT with gentle rocking, then washed with PBST.
H) To develop the plate, ABTS substrate is added to each well and incubated until a sufficiently high signal is achieved.

I) The plate is read in a plate reader at an absorbance of 405 nm. Data is then analyzed and/or visualized using any appropriate means such as graphing the data using GraphPad Prism 8.0 software (GraphPad Software Inc).

In some embodiments, the 80% competition value can be determined as outlined in Example 54 herein. In some embodiments, the 80% competition value can be determined as follows: Ab diluted 2-fold in PBS and coated a 96-well ELISA plate. After incubating the plate at 4° C. overnight, the plate is washed with PBST three times, followed by a blocking step with 2% BSA in PBST and incubation for an hour at room temperature (RT) with gentle rocking. The existing blocking solution is then discarded from the plate. Binding solutions are prepared by 2-fold dilutions from 4 μg/ml in a 2% buffer of BSA in PBST containing Flag-tagged Gal-3 protein. The dilution is then applied to the plate, then serially diluted two-fold in 2% BSA in PBST. The plate is incubated for an hour at RT with gentle rocking, then washed with PBST three times. Afterwards, HRP-conjugated anti-FLAG Gal3 antibodies are diluted to in 2% BSA in PBST and added to all the wells. The plate is incubated for 40 minutes at RT with gentle rocking, then washed with PBST three times. To develop the plate, ABTS substrate is added to each well and incubated until a sufficiently high signal is achieved. The plate is read in a plate reader at an absorbance of 405 nm. Data is then analyzed and/or visualized using any appropriate means such as graphing the data using GraphPad Prism 8.0 software (GraphPad Software Inc). If the anti-gal3 blocking antibody competes for binding with the anti-Gal3 antibody, then HRP-conjugated anti-FLAG Gal3 will not be detectable following the wash. If the anti-gal3 blocking antibody does not compete for binding with the anti-Gal3 antibody, then HRP-tagged anti-FLAG Gal3 will be detectable, or will be detectable to a lesser level following the wash.

A human or humanized antibody that competes for binding to GAL3 with TB006, wherein the antibody competes at a level of at least 80% competition. In some embodiments, the antibody is not one or any of: F847C.21H6 ((VH: QIQLVQSGPELKKPGETVKISCKASGYTFTIFGMNWVKQAPGKGLKWMGWVNTYT GEPTYADDFKGRFAFSLETSASTAYLQINNLKNEDTATYFCARGWFAYWGQGTLVT VSA (SEQ ID NO: 367); VL: DVVMTQTPLTLSVTIGQPASISCKSSRSLLDSDGKTYLNWLLQRPGQSPKRLIYLVSK LDSGVPDRFTGSGSGTDFTLKISRVEAEDLGVYYCWQGTHFPQTFGGGTKLEIN (SEQ ID NO:440)), F846TC.14A2 (VH: QIQLVQSGPELKKPGETVKISCKASGYTFTNYGMNWVKQAPGKGLKWMGWINTYT GEPTYAGDLKGRFAFSLETSASTAYLQINNLKNEDTATYFCVRYTMDYWGQGTSVT VSS (SEQ ID NO: 326); VL: QAVVTQESALTTSPGETVTLTCRSSTGAVTTSNYANWVQEKPDHLFTGLIGGINNRV PGVPARFSGSLIGDKAALTITGAQTEDEAIYFCALWYSNHWVFGGGTKLTVL (SEQ ID NO:403)), F847C.12F12 (VH: QIQLVQSGPELKKPGETVKISCKASGYTFTNYGMNWVKQAPGKGLKWMGWINTYT GEPTYVDDFKGRFAFSLETSASTAYLRINNLKNEDTATYFCAKFGNYVGAMDYWGQ GTSVTVSS (SEQ ID NO:343); VL: DIQVIQSPSSLSASLGDTITITCHASQNINVWLSWYQQKPGNIPKLLIYKASNLHTGVP SRFSGSGSGTGFTLTISSLQPEDIATYYCQQGQSYPYTFGGGTKLELK (SEQ ID NO:409)), F846T.16B5 (VH: QIQLVQSGPELKKPGETVKISCKASGYTFTNYGMNWVKQAPGKGLKWMGWINTYT GEPAYADDFKGRFAFSLETSASTAYLQINNLKNEDMATYFCARWGGYDGDYYAMD YWGQGTSVTVSS (SEQ ID NO:328); VL: NIMMTQSPSSLAVSAGEKVTMSCKSSQSVLYSSNQKNYLAWYQQKPGQSPKLLIYW ASTRESGVPDRFTGSGSGTDFTLTISSVQAEDLAVYYCHQYLSSLTFGAGTKLELK (SEQ ID NO:405)), F846C.2H3 (VH: QIQLVQSGPELKKPGETVKISCKASGYTFTNYGMNWVKQAPGKGLKWMGWINTYT GEPSYADDFKGRFAFSLETSASTAYLQINNLKNEDMATYFCARWGGYDGDYYAMD YWGQGTSVTVSS (SEQ ID NO:323); VL: NIMMTQSPSSLAVSAGEKVTMSCKSSQSVLYSSNQKNYLAWYQQKPGQSPKLLIYW ASTRESGVPDRFTGSGSGTDFTLTISSVQPEDLAVYYCHQYLSSLTFGAGTKLELK (SEQ ID NO:400)), F846C.1F5 (VH: QIQLVQSGPELKKPGETVKISCKASGYTFTNYGMNWVKQAPGKGLKWMGWINTYT GEPSYADDFKGRFAFSLETSASTAYLQINNLKNEDMATYFCARWGGYDGDYYAMD YWGQGTSVTVSS (SEQ ID NO:323); VL: NIMMTQSPSSLAVSAGEKVTMSCKSSQSVLYSSNQKNYLAWYQQKPGQSPKLLIYW ASTRESGVPDRFTGSGSGTDFTLTISSVQPEDLAVYYCHQYLSSLTFGAGTKLELK (SEQ ID NO: 400)), TB001 (VH: QVQLVQSGSELKKPGASVKVSCKASGYTFTNYGMNWVRQAPGQGLKWMGWINTN TGEPTYVEEFTGRFVFSLETSVSTAYLQISSLKAEDTAVYFCAPYDNFFAYWGQGTT VTVSS (SEQ ID NO: 297); VL: DIVLTQSPLSLPVTPGEPASISCRSSKSLLYKDGKTYLNWFLQKPGQSPQLLIYLMSTH ASGVPDRFSGSGSGTDFTLKISRVEAEDVGVYYCQQLVDYPLTFGGGTKLEIK (SEQ ID NO: 374)), or Synagis-hIgG4 (VH: QVTLRESGPALVKPTQTLTLTCTFSGFSLSTSGMSVGWIRQPPGKALEWLADIWWDD KKDYNPSLKSRLTISKDTSKNQVVLKVTNMDPADTATYYCARSMITNWYFDVWGA GTTVTVSS (SEQ ID NO: 1911); VL: DIQMTQSPSTLSASVGDRVTITCKCQLSVGYMHWYQQKPGKAPKLLIYDTSKLASG VPSRFSGSGSGTEFTLTISSLQPDDFATYYCFQGSGYPFTFGGGTKLEIK (SEQ ID NO: 1912)). In some embodiments, the 80% competition is determined as follows:
A) Ab diluted 2-fold in PBS and coated a 96-well ELISA plate.
B) After incubating the plate at 4° C. overnight, the plate is washed with PBST three times, followed by a blocking step with 2% BSA in PBST and incubation for an hour at room temperature (RT) with gentle rocking.
C) The existing blocking solution is then discarded from the plate.
D) Binding solutions are prepared by 2-fold dilutions from 4 μg/ml competing antibody in a 2% buffer of BSA in PBST containing Flag-tagged Gal-3 protein.
E) The dilution is then applied to the plate, then serially diluted two-fold in 2% BSA in PBST.
F) The plate is incubated for an hour at RT with gentle rocking, then washed with PBST three times.
G) Afterwards, HRP-conjugated anti-FLAG Gal3 antibodies are diluted to in 2% BSA in PBST and added to all the wells.
H) The plate is incubated for 40 minutes at RT with gentle rocking, then washed with PBST three times.
I) The ELISA plate is developed by adding ABTS substrate to each well and incubated until a sufficiently high signal is achieved.
J) The plate is read in a plate reader at an absorbance of 405 nm.
K) Data is then analyzed and/or visualized using any appropriate means such as graphing the data using GraphPad Prism 8.0 software (GraphPad Software Inc).
   In some embodiments, instead of the 80% competition measurement, an alternative measurement or amount of competition can be used, so long as it is equivalent to 80% (or the desired percent competition) as determined by any of the ELISA style competition assays provided herein. Thus, in some embodiment, any antibody that shows competition for binding to TB006 to GAL-3, as a level that is equivalent to any of the ELSIA competition assays provided herein can be used for any of the methods and/or compositions provided herein.

In some embodiments, the 80% competition value can be determined as outlined in Example 54 herein. In some embodiments, the 80% competition value can be determined as follows: Ab diluted 2-fold in PBS from a concentration of 4 μg/ml and coated a 96-well ELISA plate by adding 80 μl per well. After incubating the plate at 4° C. overnight, the plate is washed with 300 μl PBST three times, followed by a blocking step with 150 μl of 2% BSA in PBST per well and incubated for an hour at room temperature (RT) with gentle rocking. The existing blocking solution is then discarded from the plate. Binding solutions are prepared by 2-fold dilutions from 4 μg/ml competing antibody in a 2% buffer of BSA in PBST containing Flag-tagged Gal-3 to a concentration of 4 μg/ml. The dilution is then applied to the plate by adding 60 μl per well column-wise for each galectin-3, then serially diluted two-fold length-wise in 2% BSA in PBST. The plate is incubated for an hour at RT with gentle rocking, then washed with 300 μl PBST three times. Afterwards, HRP-conjugated anti-FLAG antibodies are diluted to 1:2000 in 2% BSA in PBST, and 25 μl is added to all the wells. The plate is incubated for 40 minutes at RT with gentle rocking, then washed with 300 μl PBST three times. To develop the plate, 50 μl of ABTS substrate is added to each well and incubated until a sufficiently high signal was achieved. The plate is read in a plate reader at an absorbance of 405 nm. Data can be graphed using GraphPad Prism 8.0 software (GraphPad Software Inc).

As noted herein, antibodies that compete for binding with TB006 at a level of at least 80% (as, for example, determined in the method of Example 54) can be used in any of the embodiments provided herein (in some embodiments). In further support that this genus of antibodies (as shown in the results of Example 54) is herein provided, it is noted that Examples 53-56 provide structural information regarding the relevant epitopes and competition aspects of numerous antibodies provided herein. Relevant epitope information has been provided by HDX (in Example 53), competition data in Example 54, and crystal structure and other functional information in Examples 55 and 56. Thus, in some embodiments, any antibodies that compete for binding to Gal3 with TB006, can be used in the methods provided herein.

As noted herein, antibodies that compete for binding with TB006 at a level of at least 50%, 60%, 70%, 80%, 90%, or 100% can be used in any of the embodiments provided herein (in some embodiments), or that competes for binding with TB006 at a level that is in a range that is defined by any two of the preceding values. For example, in some embodiments, the antibodies compete for binding with TB006 at a level between about 50%-100%, 50%-80%, 50%-70%, 60%-100%, 60%-90%, 60%-70%, 70%-100%, or 70%-90%.

In some embodiments, the human or humanized antibody comprises an antibody in Table 1.

TABLE 1

| | | SEQ | | SEQ |
|---|---|---|---|---|
| Ab | VH | ID NO | VL | ID NO |
| 13H12- | QVQLVQSGSELKKPGASVKVSCKA | 1882 | DVVMTQSPLSLPVTLGQPA | 1899 |
| VH3- | SGYTFTEYTIHWVRQAPGQGLEWM | | SISCRSSQSLFHSDGKTYL | |
| hIgG4 | GWFYPGSDNINYNEGFTGRFVLSA | | NWLQQRPGQSPRRLIYLVS | |
| (S228P)- | DKSVSTAYLQICSLKAEDTAVYFC | | KRDSGVPDRFSGSGSGTDF | |
| VL1 | ARHEEGVITTVSYFDYWGQGTLVT | | TLKISRVEAEDVGVYYCWQ | |
| | VSS | | GTHFPLTFGGGTKVEIK | |
| 4A11- | QVQLVQSGAEVKKPGASVKVSCKA | 1883 | DIVMTQTPLSLSVTPGQPA | 1900 |
| VH3- | SGYSFTNYYMHWVRQAPGQRLEWM | | SISCKSSKSLLHSDGITYL | |
| hIgG4 | GWIYPGSGNTNYNEKFQGRVTITA | | YWYLQKPGQSPQLLIYRMS | |
| (S228P)- | DTSASTAYMELSSLRSEDTAVYYC | | NLASGVPDRFSGSGSGTDF | |
| VL1 | STAPGGFDVWGQGTTVTVSS | | TLKISRVEAEDVGVYYCAQ | |
| (TB006) | | | MLEFPLTFGQGTKLEIK | |
| 19B5- | QVQLVQSGSELKKPGASVKVSCKA | 1884 | DIQMTQSPSSLSASVGDRV | 1901 |
| H1L1- | SGYTFTTYAMSWVRQAPGQGLEWM | | TITCRASQGINNYLNWYQQ | |
| hIgG4 | GWINTYSGVPTYADDFTGRFVFSL | | KPGKVPKLLIYYASTLHSG | |
| (S228P) | ETSVSTAYLQISSLKAEDTAVYFC | | VPSRFSGSGSGTDYTLTIS | |
| | ARGPYAMDYWGQGTLVTVSS | | SLQPEDVATYYCQQYSQVP | |
| | | | YTFGQGTKLEIK | |
| 14H10.2C9- | QIQLVQSGPELKKPGETVKISCKA | 1885 | DVVMTQTPLTLSVTIGQPA | 1902 |
| hIgG4 | SGYTFTTYGMGWVKQAPGKDLKWM | | SISCKSSQSLFDSDGKTYL | |
| (S228P) | GWINTYSGVPTYADDFKGRFAFSL | | NWLLQRPGQSPKRLIYLVS | |
| | ETSASTAYLQISNLKNEDTATYFC | | KLDSGVPDRFTGSGSGTDF | |
| | STPYEYDGAYWGQGTLVTVSA | | TLKISRVEAEDLGVYYCWQ | |
| | | | GTHFPLTFGAGTKLEMK | |
| 15G7.2A7- | EVQLQQSGPELVKPGASVKISCKA | 1886 | DIQMNQSPSSLSASLGDTI | 1903 |
| hIgG4 | SGYTFTDYYMNWVKQSHGKSLEWI | | SITCRASQNINIWLSWYQQ | |
| (S228P) | GDINPNNGGTNYNQKFKGKATLTV | | KPGNIPQLLIYKASNLHTG | |
| | DKSSSTAYMELRSLTSEDSAVYYC | | VPSRFSGSGSGTDFTLTIS | |
| | TSGYGFPYWGQGTLVTVSA | | SLQPEDIATYYCLQGQSYP | |
| | | | LTFGAGTKLVMK | |
| MOPC21- | DVQLVESGGGLVQPGGSRKLSCAA | 1887 | NIVMTQSPKSMSMSVGERV | 1904 |
| hIgG4 | SGFTFSSFGMHWVRQAPEKGLEWV | | TLTCKASENVVTYVSWYQQ | |
| (S228P) | AYISSGSSTLHYADTVKGRFTISR | | KPEQSPKLLIYGASNRYTG | |
| | DNPKNTLFLQMTSLRSEDTGMYYC | | VPDRFTGSGSATDFTLTIS | |
| | ARWGNYPYYAMDYWGQGTSVTVSS | | SVQAEDLADYHCGQGYSYP | |
| | | | YTFGGGTKLEIK | |
| 846.1H12- | QIQLVQSGPELKKPGETVKISCKA | 1888 | METDTLLLWVLLLWVPGST | 1905 |
| hIgG4 | SGYTFTNFGMNWVKQAPGKGLKWM | | GNIMMTQSPSSLAVSAGQK | |
| (S228P) | GWINTYTGEPSYADDFKGRFAFSL | | VTMSCKSSQSVLYSSSQKN | |
| | ETSASTAYLQINNLKNEDIATYFC | | YLAWYQQKPGQSPKLLIYW | |
| | ARWGGYAGDYYAMDYWGQGTSVTV | | ASTRESGVPDRFSGSGSGT | |
| | SS | | DFTLTISSVQPEDLAVYYC | |
| | | | HQYLSSLTFGAGTKLELK | |
| 846.2H3- | QIQLVQSGPELKKPGETVKISCKA | 1889 | NIMMTQSPSSLAVSAGQKV | 1906 |
| hIgG4 | SGYTFTNYGMNWVKQAPGKGLKWM | | TMSCKSSQSVLYSSSQKNY | |
| (S228P) | GWINTYTGEPSYADDEKGRFAFSL | | LAWYQQKPGQSPKLLIYWA | |
| Sister | ETSASTAYLQINNLKNEDMATYFC | | STRESGVPDRFSGSGSGTD | |
| clone- | ARWGGYDGDYYAMDYWGQGTSVTV | | FTLTISSVQPEDLAVYYCH | |
| IMTAB0201 | SS | | QYLSSLTFGAGTKLELK | |
| 846.1F5-4 | QIQLVQSGPELKKPGETVKISCKA | 1890 | NIMMTQSPSSLAVSAGEKV | 1907 |
| hIgG | SGYTFTNYGMNWVKQAPGKGLKWM | | TMSCKSSQSVLYSSNQKNY | |
| (S228P) | GWINTYTGEPSYADDFKGRFAFSL | | LAWYQQKPGQSPKLLIYWA | |
| Sister | ETSASTAYLQINNLKNEDMATYFC | | STRESGVPDRFTGSGSGTD | |
| clone- | ARWGGYDGDYYAMDYWGQGTSVTV | | FTLTISSVQPEDLAVYYCH | |
| IMTAB0199 | SS | | QYLSSLTFGAGTKLELK | |
| 846T.16B5- | QIQLVQSGPELKKPGETVKISCKA | 1891 | NIMMTQSPSSLAVSAGEKV | 1908 |
| hIgG4 | SGYTFTNYGMNWVKQAPGKGLKWM | | TMSCKSSQSVLYSSNQKNY | |
| (S228P) | GWINTYTGEPAYADDEKGRFAFSL | | LAWYQQKPGQSPKLLIYWA | |
| | ETSASTAYLQINNLKNEDMATYFC | | STRESGVPDRFTGSGSGTD | |
| | ARWGGYDGDYYAMDYWGQGTSVTV | | FTLTISSVQAEDLAVYYCH | |
| | SS | | QYLSSLTFGAGTKLELK | |
| 20D11.2C6- | EVQLQQSGPELVKPGASVKISCKA | 1892 | DIQMNQSPSSLSASLGDTI | 1909 |
| hIgG4 | SGYTFTDFYINWVKQSHGKSLEWI | | TITCRASQNIYIWLSWYQQ | |
| (S228P) | GDINPKNGGINYNPKFKIKATLTV | | KPGNIPKLLIYKASNLHTG | |
| | DKSSSTSYMDLRGLTSEDSAVYYC | | VPSRFSGSGSGTDFTLTIS | |
| | TSGYGFPYWGQGTLVTVSA | | TLQPEDIATYFCLQGQSYP | |
| | | | LTFGAGTKLEMK | |
| IMT001- | QVQLVQSGSELKKPGASVKVSCKA | 1893 | DIVLTQSPLSLPVTPGEPA | 1910 |
| 4-HCLC | SGYTFTNYGMNWVRQAPGQGLKWM | | SISCRSSKSLLYKDGKTYL | |
| (TB001) | GWINTNTGEPTYVEEFTGRFVFSL | | NWFLQKPGQSPQLLIYLMS | |
| | ETSVSTAYLQISSLKAEDTAVYFC | | THASGVPDRFSGSGSGTDF | |
| | APYDNFFAYWGQGTTVTVSS | | TLKISRVEAEDVGVYYCQQ | |
| | | | LVDYPLTFGGGTKLEIK | |
| 847.12F12- | QIQLVQSGPELKKPGETVKISCKA | 1894 | DIQVIQSPSSLSASLGDTI | 1911 |
| hIgG4 | SGYTFTNYGMNWVKQAPGKGLKWM | | TITCHASQNINVWLSWYQQ | |
| (S228P) | GWINTYTGEPTYVDDFKGRFAFSL | | KPGNIPKLLIYKASNLHTG | |
| | ETSASTAYLRINNLKNEDTATYFC | | VPSRFSGSGSGTGFTLTIS | |
| | AKFGNYVGAMDYWGQGTSVTVSS | | SLQPEDIATYYCQQGQSYP | |
| | | | YTFGGGTKLELK | |
| 846T.14A2- | QIQLVQSGPELKKPGETVKISCKA | 1895 | QAVVTQESALTTSPGETVT | 1912 |
| hIgG4 | SGYTFTNYGMNWVKQAPGKGLKWM | | LTCRSSTGAVTTSNYANWV | |
| (S338P) | GWINTYTGEPTYAGDLKGRFAFSL | | QEKPDHLFTGLIGGINNRV | |
| | ETSASTAYLQINNLKNEDTATYFC | | PGVPARFSGSLIGDKAALT | |
| | VRYTMDYWGQGTSVTVSS | | ITGAQTEDEAIYFCALWYS | |
| | | | NHWVFGGGTKLTVL | |
| 798- | QIQLVQSGPELKKPGESVKISCKA | 1896 | DIQMTQTTSSLSASLGDRV | 1913 |
| 9.20H5.A3- | SGYAFTTYGMSWVQQAPGKGLKWM | | TINCSASQDISNYLNWYQQ | |
| mH1mL0- | GWVNTYSGVPTYADDFKGRFAFSL | | KPDGTVKLLIYYTSSLLSG | |
| hIgG4 | ETSASTAYLQINNLRNEDTATYFC | | VPSRFSGSGSGTDYSLTIS | |
| (S228P) | ARGPYAMDYWGQGTSVTVSS | | NLEPEDIATYFCQQYSKLP | |
| | | | YTFGSGTHLEIK | |
| F847C.21H6- | QIQLVQSGPELKKPGESVKISCKA | 1897 | DVVMTQTPLTLSVTIGQPA | 1914 |
| hIgG4 | SGYAFTTYGMSWVQQAPGKGLKWM | | SISCKSSRSLLDSDGKTYL | |
| (S228P) | GWVNTYSGVPTYADDFKGRFAFSL | | NWLLQRPGQSPKRLIYLVS | |
| | ETSASTAYLQINNLRNEDTATYFC | | KLDSGVPDRFTGSGSGTDF | |
| | ARGPYAMDYWGQGTSVTVSS | | TLKISRVEAEDLGVYYCWQ | |
| | | | GTHFPQTFGGGTKLEIN | |
| 798- | QIQLVQSGPELKKPGETVKISCKA | 1898 | DALMTQTPLTLSVTIGQPA | 1915 |
| 9.19D9.2E5- | SGYTFTTYGMGWVKQAPGKGLKWM | | SISCKSSQSLLHSDGKTYL | |
| hIqG4 | GWINTYSGVPTYADDFKGRFAFSL | | NWLLQRPGQPPKRLMYLVS | |
| (S228P)- | ETSTSTAYLQINNLKNEDMATYFC | | TLDSGVPDRFTGSGSGTDF | |
| mV2 | ATPYEYDGAYWGQGTLVTVSA | | TLKISRVEAEDLGVYYCWQ | |
| | | | GTHFPLTFGAGTKLELK | |

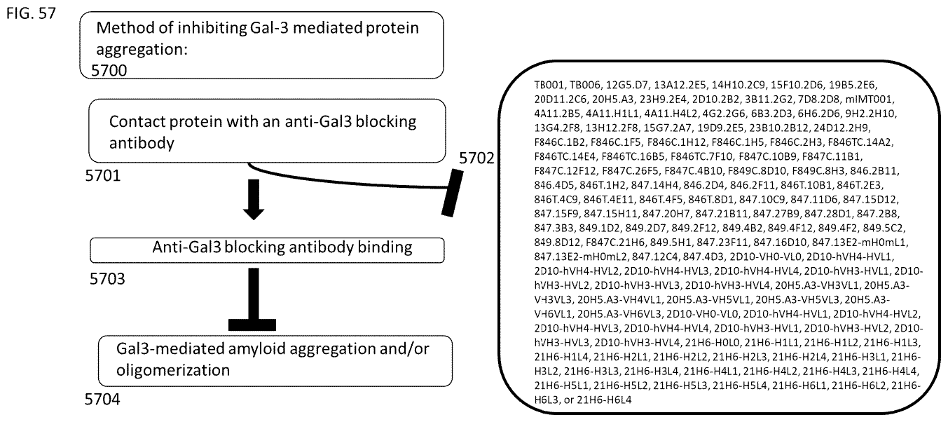

FIG. 57 is a flow chart depicting some embodiments of methods for inhibiting Gal3-mediated aggregation of a protein.

In some embodiments, a method of inhibiting Gal3-mediated oligomerization of a protein is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated oligomerization of the protein.

In some embodiments, a method of inhibiting Gal3-mediated amyloid aggregation of a protein is disclosed. In some embodiments, the method comprises: contacting the protein with an anti-Gal3 antibody or binding fragment thereof, wherein binding of the anti-Gal3 antibody or binding fragment thereof to Gal3 inhibits Gal3-mediated oligomerization of the protein.

In some embodiments are disclosed methods of inhibiting Gal3-mediated aggregation or oligomerization of a protein 5700. In some embodiments, the methods comprise contacting the protein with an anti-Gal3 blocking antibody or binding fragment thereof 5701. In some embodiments, the anti-Gal3 blocking antibody blocks binding of one or more anti-Gal3 antibodies 5702 from binding to Gal3. In some embodiments, binding of the anti-Gal3 blocking antibody 5703 or binding fragment thereof to Gal3 inhibits Gal3-mediated aggregation or oligomerization of the protein 5704. In some embodiments, the protein is in a cell. In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein.

Also disclosed herein are methods of inhibiting Gal3-mediated aggregation or oligomerization of a protein in a cell. In some embodiments, the methods comprise contacting the cell with an anti-Gal3 blocking antibody or binding fragment thereof. In some embodiments, binding of the anti-Gal3 blocking antibody or binding fragment thereof to Gal3 in the cell inhibits Gal3-mediated aggregation or oligomerization of the protein.

In some embodiments, the method is performed in vitro or in vivo.

In some embodiments, Gal3-mediated aggregation or oligomerization of the protein is inhibited by at least 5%, 10%, 15%, 20%, 25%, 30%, 35%, 40%, 45%, 50%, 55%, 60%, 65%, 70%, 75%, 80%, 85%, 90%, 95%, or 99%, or any percentage within a range defined by any two of the aforementioned percentages, after contacting with the anti-Gal3 blocking antibody or binding fragment thereof relative to a cell that is not contacted with the anti-Gal3 antibody blocking or binding fragment thereof.

In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein. In some embodiments, the phosphorylated tau forms trimers, tetramers, or higher order oligomers when contacted with Gal3.

In some embodiments, the anti-Gal3 blocking antibody or binding fragment competes for binding to Gal3 with an anti-Gal3 antibody or binding fragment thereof comprising (1) a heavy chain variable region comprising a VH-CDR1, a VH-CDR2, and a VH-CDR3; and (2) a light chain variable region comprising a VL-CDR1, a VL-CDR2, and a VL-CDR3. In some embodiments, the anti-Gal3 blocking antibody or binding fragment competes for binding to Gal3 with an anti-Gal3 antibody or binding fragment thereof, wherein the VH-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70. In some embodiments, the anti-Gal3 blocking antibody or binding fragment competes for binding to Gal3 with an anti-Gal3 antibody or binding fragment thereof, wherein the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952. In some embodiments, the anti-Gal3 blocking antibody or binding fragment competes for binding to Gal3 with an anti-Gal3 antibody or binding fragment thereof, wherein the VH-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802, 953, 954. In some embodiments, the anti-Gal3 blocking antibody or binding fragment competes for binding to Gal3 with an anti-Gal3 antibody or binding fragment thereof, wherein the VL-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220. In some embodiments, the anti-Gal3 blocking antibody or binding fragment competes for binding to Gal3 with an anti-Gal3 antibody or binding fragment thereof, wherein the VL-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 211-247. In some embodiments, the anti-Gal3 blocking antibody or binding fragment competes for binding to Gal3 with an anti-Gal3 antibody or binding fragment thereof, wherein the VL-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296. In some embodiments, the anti-Gal3 blocking antibody or binding fragment competes for binding to Gal3 with an anti-Gal3 antibody or binding fragment thereof, wherein the anti-Gal3 antibody or binding fragment thereof comprises a combination of the VH-CDR1, VH-CDR2, VH-CDR3, VL-CDR1, VL-CDR2, and VL-CDR3 as illustrated in FIG. 13. In some embodiments, the anti-Gal3 blocking antibody or binding fragment competes for binding to Gal3 with an anti-Gal3 antibody or binding fragment thereof, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 blocking antibody or binding fragment competes for binding to Gal3 with an anti-Gal3 antibody or binding fragment thereof, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464. In some embodiments, the anti-Gal3 blocking antibody or binding fragment competes for binding to Gal3 with an anti-Gal3 antibody or binding fragment thereof, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489. In some embodiments, the anti-Gal3 blocking antibody or binding fragment competes for binding to Gal3 with an anti-Gal3 antibody or binding fragment thereof, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514. In some embodiments, any antibody (human, humanized, or not) that competes for binding to any one or more of the proceeding antibodies (optionally at, at least 80% competition as defined in the present examples), can be used in the method as well. In some embodiments, the antibody binds to the same or overlapping epitope of any one or more of the preceding antibodies.

In some embodiments, the anti-Gal3 blocking antibody or binding fragment competes for binding to Gal3 with an anti-Gal3 antibody or binding fragment thereof, wherein the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or a binding fragment thereof. In some embodiments, the anti-Gal3 blocking antibody or binding fragment competes for binding to Gal3 with an anti-Gal3 antibody or binding fragment thereof, wherein the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof. In some embodiments, the anti-Gal3 blocking antibody or binding fragment competes for binding to Gal3 with an anti-Gal3 antibody or binding fragment thereof, wherein the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or a binding fragment thereof. In some embodiments, any antibody (human, humanized, or not) that competes for binding to any one or more of the proceeding antibodies (optionally at, at least 80% competition as defined in the present examples), can be used in the method as well. In some embodiments, the antibody binds to the same or overlapping epitope of any one or more of the preceding antibodies.

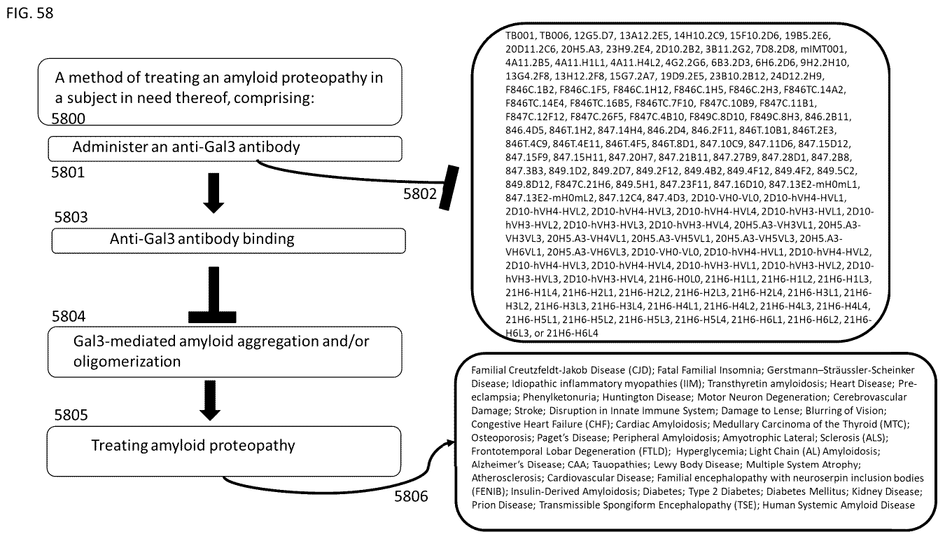

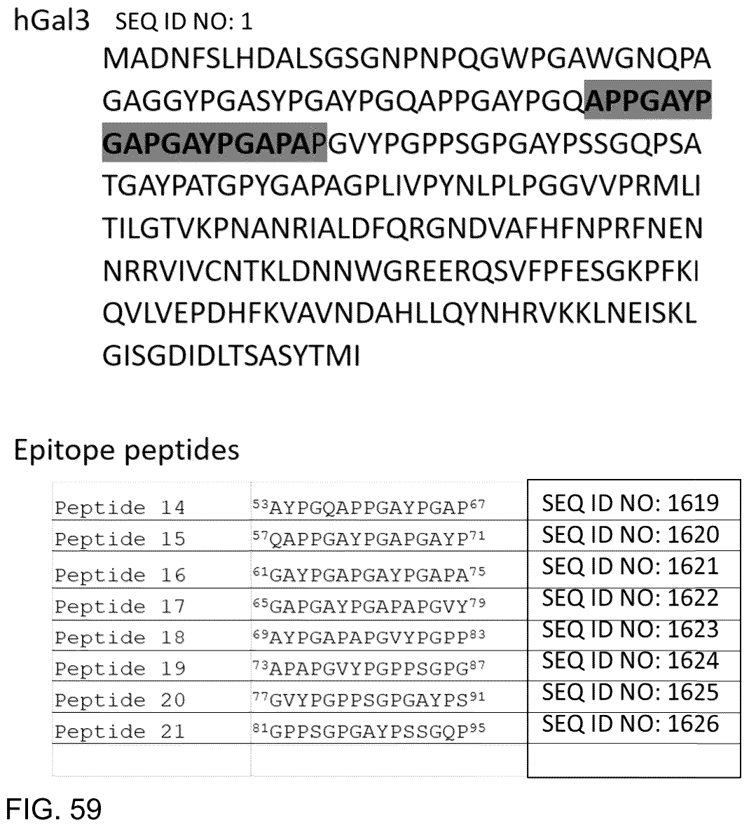

FIG. 58 is a flow chart depicting embodiments of a method of treating amyloid proteopathy in a subject.

In some embodiments are disclosed methods of treating an amyloid proteopathy in a subject in need thereof 5800. In some embodiments, the method comprises administering to the subject an anti-Gal3 blocking antibody 5801 or binding fragment thereof, wherein the anti-Gal3 blocking antibody or binding fragment thereof is a blocking antibody that blocks an anti-Gal3 antibody 5802 from binding to Gal3; wherein binding of the anti-Gal3 blocking antibody, or binding fragment thereof, binding to Gal3 5803 in the subject inhibits Gal3-mediated amyloid aggregation 5804 of a protein in the subject, thereby treating the amyloid proteopathy 5805 in the subject. In some embodiments, the amyloid proteopathy 5806 comprises familial Creutzfeldt-Jakob disease (CJD), Alzheimer's disease, CAA, tauopathies, Lewy body disease, multiple system atrophy, atherosclerosis, cardiovascular disease, familial encephalopathy with neuroserpin inclusion bodies (FENIB), insulin-derived amyloidosis, diabetes, type 2 diabetes, diabetes mellitus, kidney disease, prion disease, transmissible spongiform encephalopathy (TSE), human systemic amyloid disease, fatal familial insomnia, Gerstmann-Straussler-Scheinker disease, idiopathic inflammatory myopathies (IIM), transthyretin amyloidosis, heart disease, pre-eclampsia, phenylketonuria, Huntington disease, motor neuron degeneration, cerebrovascular damage, stroke disruption in innate immune system, damage to lenses, blurring of vision, congestive heart failure (CHF), cardiac amyloidosis, medullary carcinoma of the thyroid (MTC), osteoporosis, Paget's disease, peripheral amyloidosis, amyotrophic lateral sclerosis (ALS), frontotemporal lobar degeneration (FTLD), hyperglycemia, light chain amyloidosis (AL), a synucleinopathy, Parkinson's disease, dementia with Lewy bodies, progressive supranuclear palsy, corticobasal degeneration, Pick's disease, TDP-43 proteopathy, TTR amyloidosis (ATTR), uromodulin-associated kidney disease, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation, or any combination thereof.

In some embodiments, the protein is in a cell. In some embodiments, the protein comprises α-synuclein, tau protein, phospho tau, TAR DNA binding protein (TDP-43), transthyretin, uromodulin, islet amyloid polypeptide (IAPP), serum amyloid A (SAA), p53, apolipoprotein E (APOE), APOE-4, prion protein, fibrin, neurofilament light (NFL), CRP, SUMO, light chain, platelet-derived growth factor receptor (PDGFR), melanoma cell adhesion molecule (MCAM), complement protein C3, complement protein C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, crystallin AA, crystallin AB, cystatin-C, myostatin pro-peptide, Atrial Natriuretic Peptide (ANP), B-Type Natriuretic Peptide (BNP), or any combination therein.

In some embodiments, the anti-Gal3 blocking antibody, or binding fragment thereof, blocks binding of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, and/or 21H6-H6L4, to Gal-3. In some embodiments, any antibody (human, humanized, or not) that competes for binding to any one or more of the proceeding antibodies (optionally at, at least 80% competition as defined in the present examples), can be used in the method as well. In some embodiments, the antibody binds to the same or overlapping epitope of any one or more of the preceding antibodies.

In some embodiments, the anti-Gal3 blocking antibody blocks anti-Gal3 antibody binding to Gal3 by about 50%, 60%, 70% 75%, 80%, 81%, 82%, 83%, 84%, 85%, 86%, 87%, 88%, 89%, 90%, 91%, 92%, 93%, 94%, 95%, 96%, 97%, 98%, or 99% as compared to anti-Gal3 antibody binding in the absence of the anti-Gal3 blocking antibody, or blocks anti-Gal3 antibody binding to Gal3 by a range that is defined by any two of the preceding values. For example, in some embodiments, the anti-Gal3 blocking antibody blocks anti-Gal3 antibody binding by between about 50% and 100%, 50% and 95%, 50% and 90%, 50% and 85%, 50% and 80%, 50% and 75%, 50% and 70%, 50% and 60%, 60% and 100%, 60% and 95%, 60% and 90%, 60% and 85%, 60% and 80%, 60% and 75%, 75% and 100%, 75% and 95%, 75% and 90%, or 75% and 85%, as compared to anti-Gal3 antibody binding in the absence of the anti-Gal3 blocking antibody.

In some embodiments, the anti-Gal3 blocking antibody binds to one or more of the same epitopes as the anti-Gal3 antibody.

In some embodiments, the anti-Gal3 blocking antibody inhibits Gal3-mediated amyloid aggregation with at least 50%, 60%, 70%, 80%, 90%, 100% efficiency as compared to anti-Gal3 antibody binding in the absence of anti-Gal3 blocking antibody, or with an efficiency in a range that is defined by any two of the preceding values. For example, in some embodiments, binding of the anti-Gal3 blocking antibody inhibits Gal3-mediated amyloid aggregation with at least 50%-100%, 50%-90%, 50%-80%, 50%-70%, or 70%-100% efficiency as compared to anti-Gal3 antibody binding in the absence of anti-Gal3 blocking antibody. In some embodiments, the anti-Gal3 blocking antibody inhibits Gal3-mediated amyloid aggregation with at least 1-fold, 2-fold, 3-fold, 4-fold, 5-fold, 6-fold, 7-fold, 8-fold, 9-fold, or 10-fold efficiency as compared to anti-Gal3 antibody binding in the absence of anti-Gal3 blocking antibody, or with an efficiency in a range that is defined by any two of the preceding values. For example, in some embodiments, binding of the anti-Gal3 blocking antibody inhibits Gal3-mediated amyloid aggregation with at least 1-fold to 10-fold, 1-fold to 7-fold, 1-fold to 5-fold, 1 fold-3 fold, 3-fold to 10-fold, 3-fold to 7-fold, 3-fold to 5-fold, 5-fold to 10-fold, or 5-fold to 7-fold, efficiency as compared to anti-Gal3 antibody binding in the absence of anti-Gal3 blocking antibody.

In some embodiments, administration of the anti-Gal3 blocking antibody treats the Gal3-mediated amyloid proteopathy in the subject with at least 50%, 60%, 70%, 80%, 90%, 100% efficiency as compared to anti-Gal3 antibody in the absence of anti-Gal3 blocking antibody, or with an efficiency in a range that is defined by any two of the preceding values. For example, in some embodiments, administration of the anti-Gal3 blocking antibody treats the Gal3-mediated amyloid proteopathy in the subject with at least 50%-100%, 50%-90%, 50%-80%, 50%-70%, or 70%-100% efficiency as compared to anti-Gal3 antibody in the absence of anti-Gal3 blocking antibody. In some embodiments, administration of the anti-Gal3 blocking antibody treats the Gal3-mediated amyloid proteopathy in the subject with at least 1-fold, 2-fold, 3-fold, 4-fold, 5-fold, 6-fold, 7-fold, 8-fold, 9-fold, or 10-fold efficiency as compared to anti-Gal3 antibody binding in the absence of anti-Gal3 blocking antibody, or with an efficiency in a range that is defined by any two of the preceding values. For example, in some embodiments, administration of the anti-Gal3 blocking antibody treats the Gal3-mediated amyloid proteopathy in the subject with at least 1-fold to 10-fold, 1-fold to 7-fold, 1-fold to 5-fold, 1 fold-3 fold, 3-fold to 10-fold, 3-fold to 7-fold, 3-fold to 5-fold, 5-fold to 10-fold, or 5-fold to 7-fold, efficiency as compared to anti-Gal3 antibody binding in the absence of anti-Gal3 blocking antibody.

### Protein Aggregate Compositions and Kits

Also disclosed herein are compositions comprising a protein and Gal3. In some embodiments, Gal3 promotes amyloid aggregation and/or oligomerization of the protein in the composition. In some embodiments, the protein and Gal3 are in aqueous solution, or are dried and/or lyophilized. In some embodiments, the protein α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, or any combination thereof. In some embodiments, the tau protein is 4R tau and/or phosphorylated tau (phospho tau). In some embodiments, the phosphorylated tau is phospho-tau (S396). In some embodiments, the phosphorylated tau forms trimers, tetramers, or higher order oligomers when contacted with Gal3.

Also disclosed herein are kits comprising any of the compositions disclosed herein, such as the compositions comprising the protein and Gal3. The compositions and kits provided herein may be used for biological assays studying amyloid aggregation and/or oligomerization, which has been implicated in various pathologies. As demonstrated herein, Gal3 promotes aggregation and/or oligomerization of the proteins on a time scale that is much faster than conventional methods. For example, in some embodiments, amyloid aggregation and/or oligomerization of tau protein is intended to be achieved more rapidly compared to spontaneous aggregation and/or oligomerization of tau protein alone, or aggregation and/or oligomerization of tau protein when mixed with heparin and/or arachnoid acid. In some embodiments, amyloid aggregation and/or oligomerization of the tau protein is intended to be achieved more rapidly compared to aggregation and/or oligomerization of tau protein when mixed with heparin and/or arachnoid acid at 37° C. or about 37° C. In some embodiments, amyloid aggregation and/or oligomerization of the tau protein is intended to be achieved with no more than 0.5, 1, 2, 3, 4, 5, 6, 7, 8, 9, 10, 11, 12, 13, 14, 15, 16, 17, 18, 19, 20, 21, 22, 23, or 24 hours of contacting the tau protein with Gal3. This aggregation and/or oligomerization of tau protein by Gal3 occurs faster than previous methods, such as those involving the use of heparin and/or arachnoid acid. The kits disclosed herein may further include instructions detailing the time scales and temperatures to accomplish amyloid aggregation and/or oligomerization with the use of Gal3.

In some embodiments, the protein of the compositions or kits is intended to be contacted with Gal3 at a temperature of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any temperature within a range defined by any two of the aforementioned temperatures. In some embodiments, the protein of the compositions or kits is intended to be is contacted with Gal3 at body temperature, 37° C., or about 37° C. In some embodiments, the protein of the compositions or kits is intended to be is contacted with Gal3 below body temperature, below 37° C., or below about 37° C. In some embodiments, the protein of the compositions or kits is intended to be is contacted with Gal3 at room temperature or about room temperature. In some embodiments, the protein of the compositions or kits is intended to be is contacted with Gal3 at a temperature of about 18, 19, 20, 21, 22, 23, or 24° C., or any temperature within a range defined by any two of the aforementioned temperatures.

Accordingly, the compositions and kits provided herein have an advantage over alternative methods of forming protein aggregates and/or oligomers for study. Diseases that these compositions and kits may be useful for study are provided in the present disclosure, and may include a synucleinopathy, Parkinson's disease, dementia with Lewy bodies, multiple system atrophy, tauopathy, Alzheimer's disease, progressive supranuclear palsy, corticobasal degeneration, Pick's disease, TDP-43 proteopathy, amyotrophic lateral sclerosis, frontotemporal lobar degeneration, TTR amyloidosis (ATTR), cardiac amyloidosis, uromodulin-associated kidney disease, IAPP amyloidosis, SAA amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation, or any combination thereof.

### Pharmaceutical Formulations

A pharmaceutical formulation for treating a disease, such as in embodiments of the methods described herein, can comprise an anti-Gal3 antibody or binding fragment thereof described supra. The anti-Gal3 antibody or binding fragment thereof can be formulated for systemic administration. Alternatively, the anti-Gal3 antibody or binding fragment thereof can be formulated for parenteral administration.

In some embodiments, an anti-Gal3 antibody or binding fragment thereof is formulated as a pharmaceutical composition for administration to a subject by, but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for systemic administration. In other instances, the pharmaceutical composition describe herein is formulated for oral administration. In still other instances, the pharmaceutical composition describe herein is formulated for intranasal administration.

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multi-particulate formulations (e.g., nanoparticle formulations), and mixed immediate and controlled release formulations.

### Therapeutic Regimens

In some embodiments, the anti-Gal3 antibodies or binding fragments thereof disclosed herein are administered for therapeutic applications, such as in embodiments of the methods disclosed herein. In some embodiments, the anti-Gal3 antibody or binding fragment thereof is administered once per day, twice per day, three times per day or more. The anti-Gal3 antibody or binding fragment thereof is administered daily, every day, every alternate day, five days a week, once a week, every other week, two weeks per month, three weeks per month, once a month, twice a month, three times per month, or more. In some embodiments, the anti-Gal3 antibody or binding fragment thereof is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the anti-Gal3 antibody or binding fragment thereof is given continuously; alternatively, the dose of the anti-Gal3 antibody or binding fragment thereof being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the treated disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Polynucleotides and Vectors

In some embodiments, the present disclosure provides isolated nucleic acids encoding any of the anti-Gal3 antibodies or binding fragments thereof disclosed herein. In another embodiment, the present disclosure provides vectors comprising a nucleic acid sequence encoding any anti-Gal3 antibody or binding fragment thereof disclosed herein. In some embodiments, this disclosure provides isolated nucleic acids that encode heavy chain variable regions, light chain variable regions, heavy chains, or light chains of an anti-Gal3 antibody or binding fragment thereof disclosed herein.

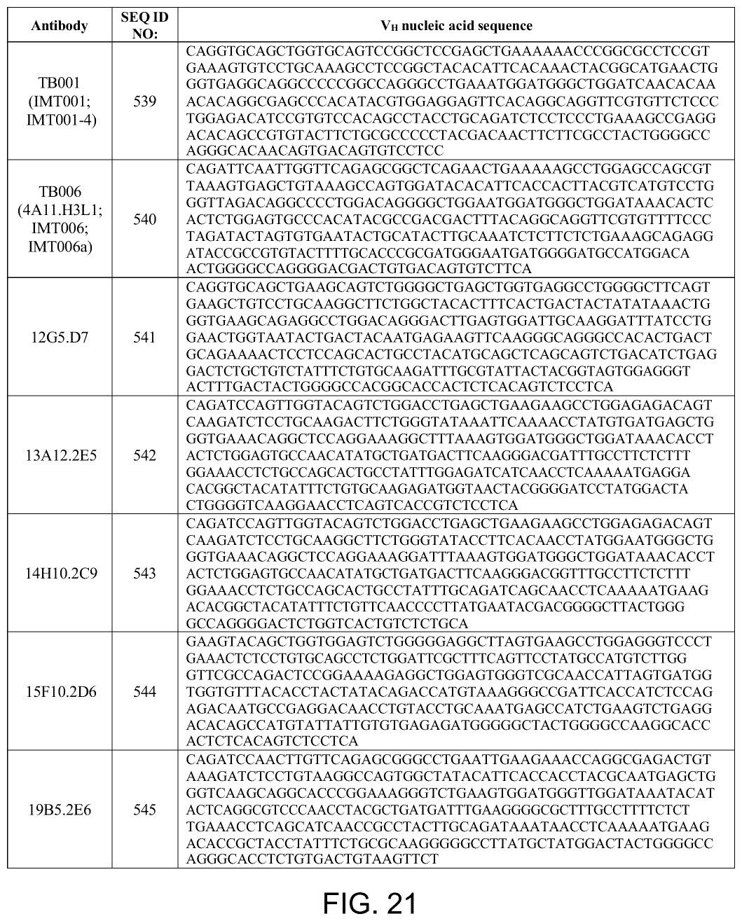

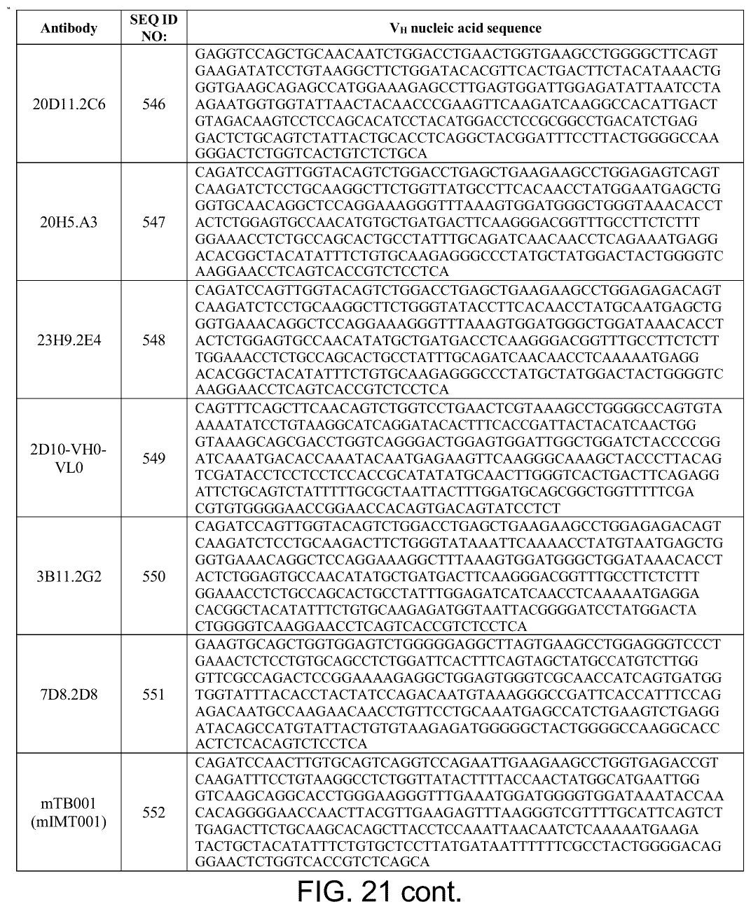

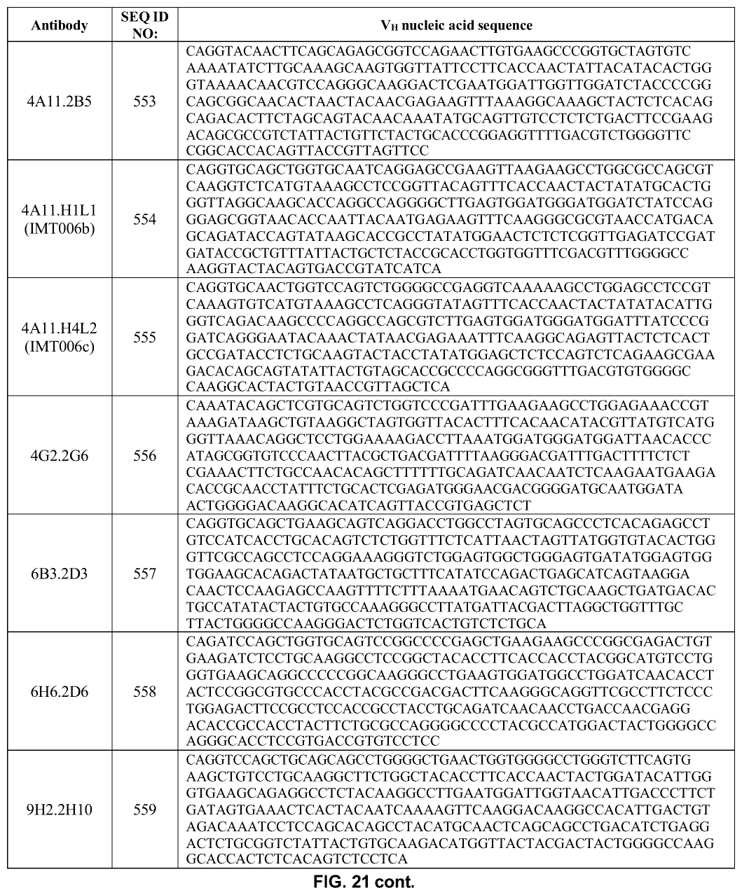

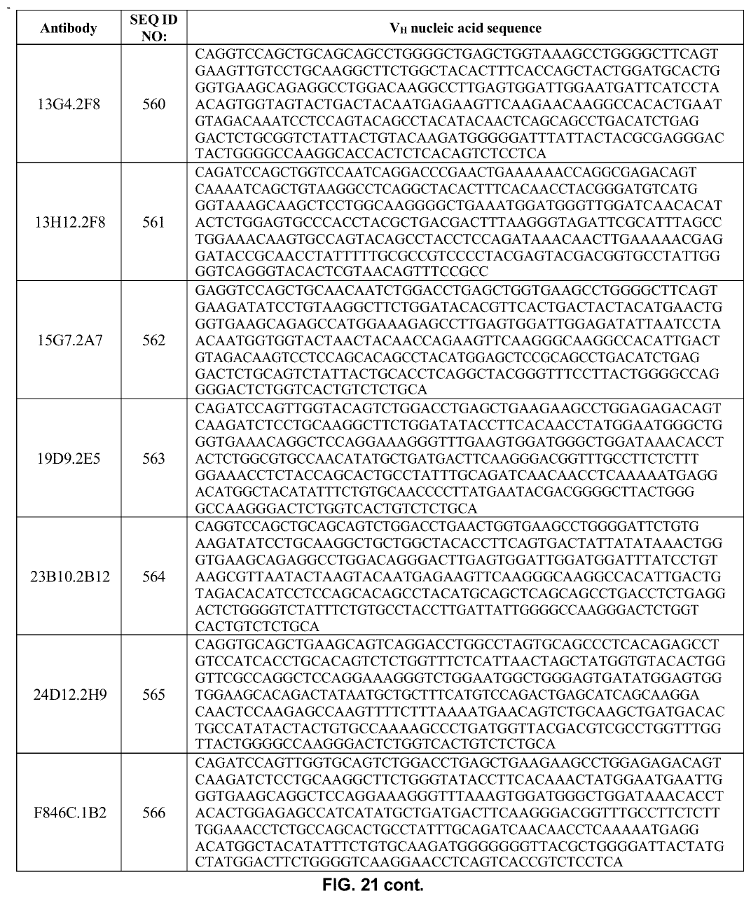

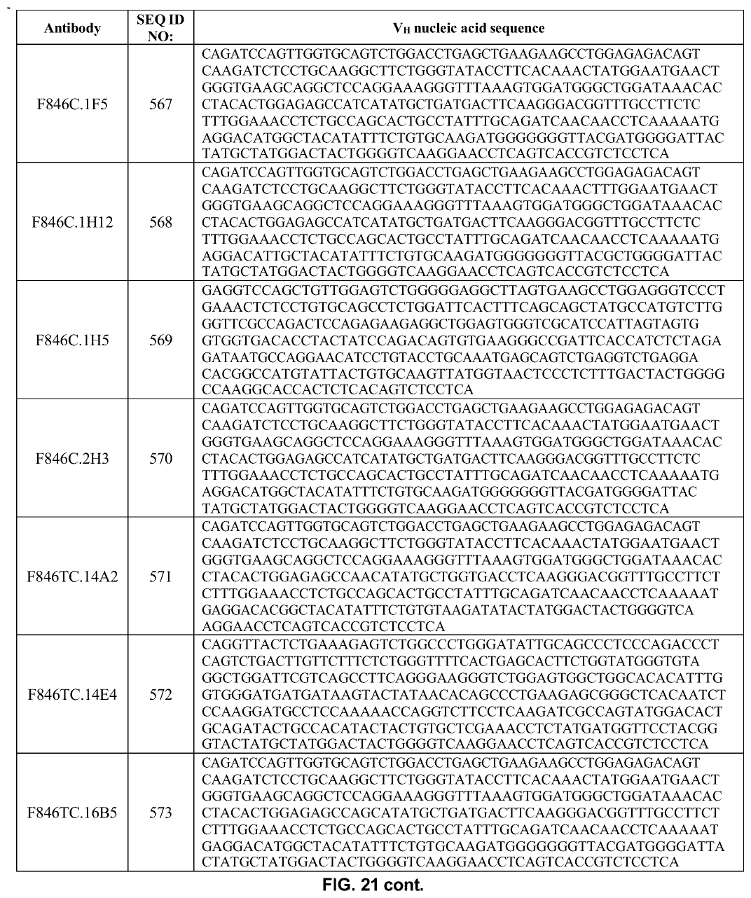

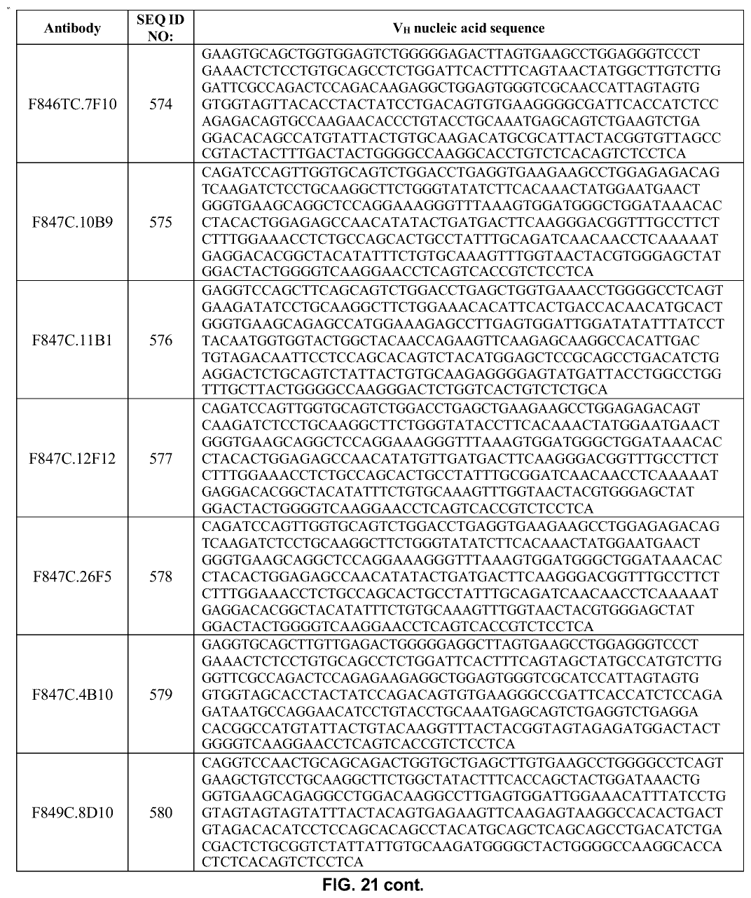

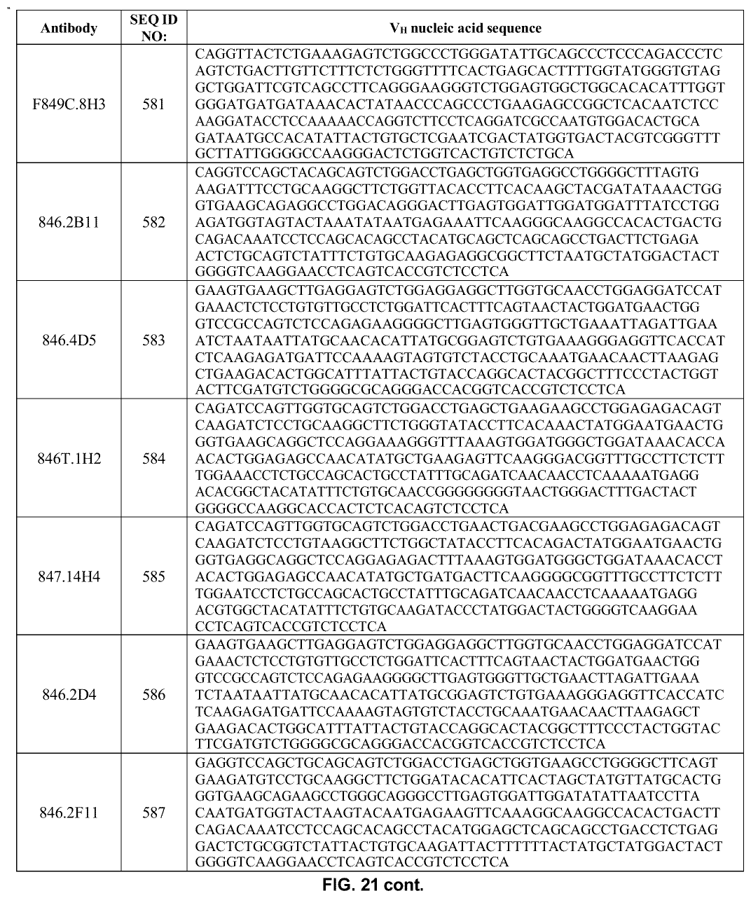

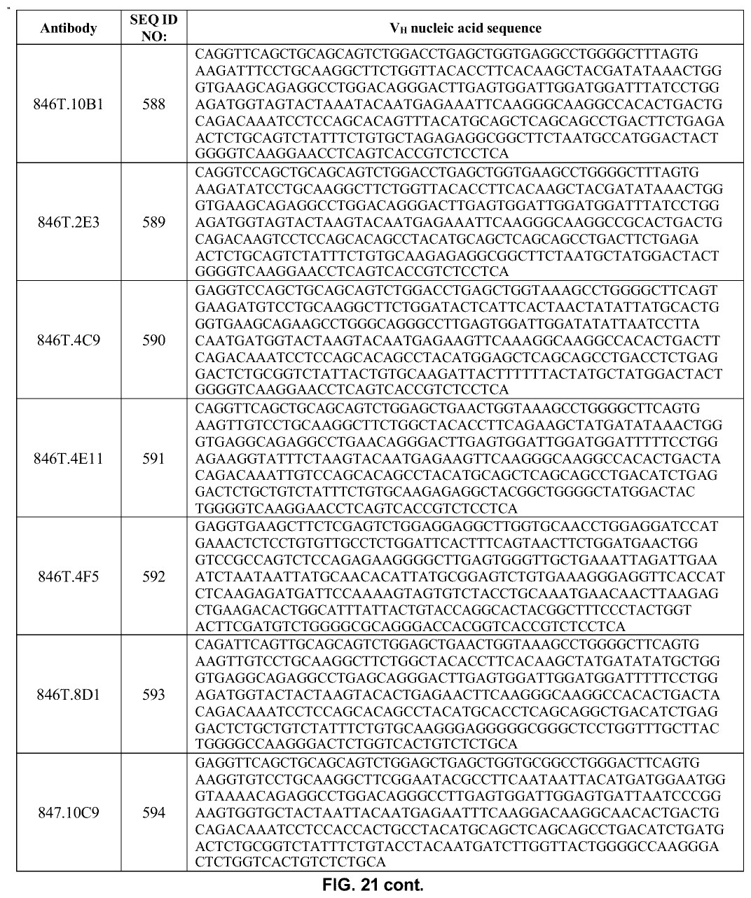

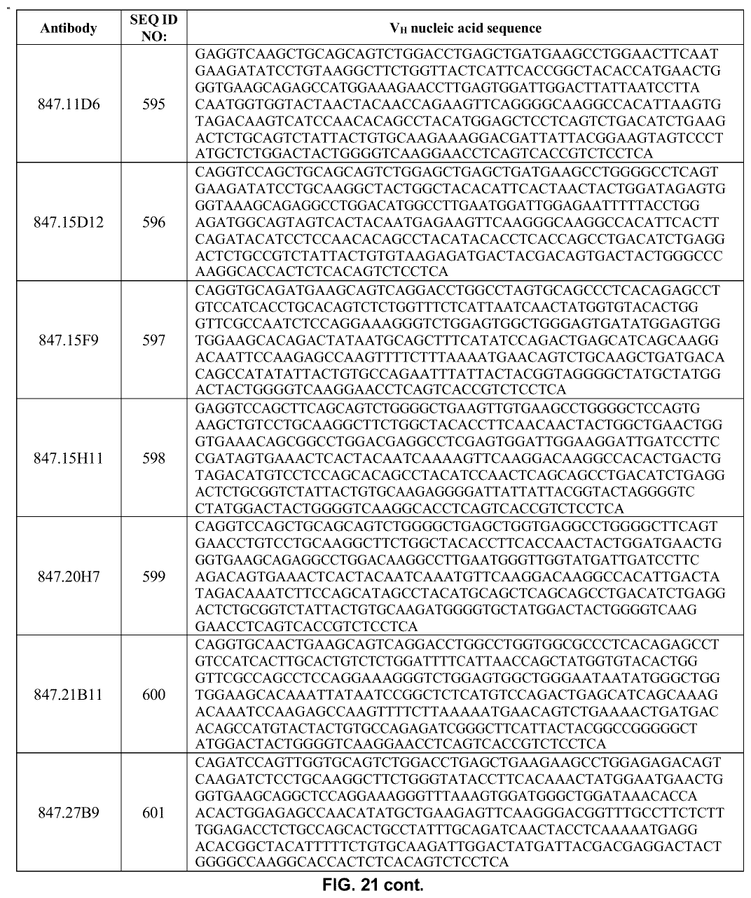

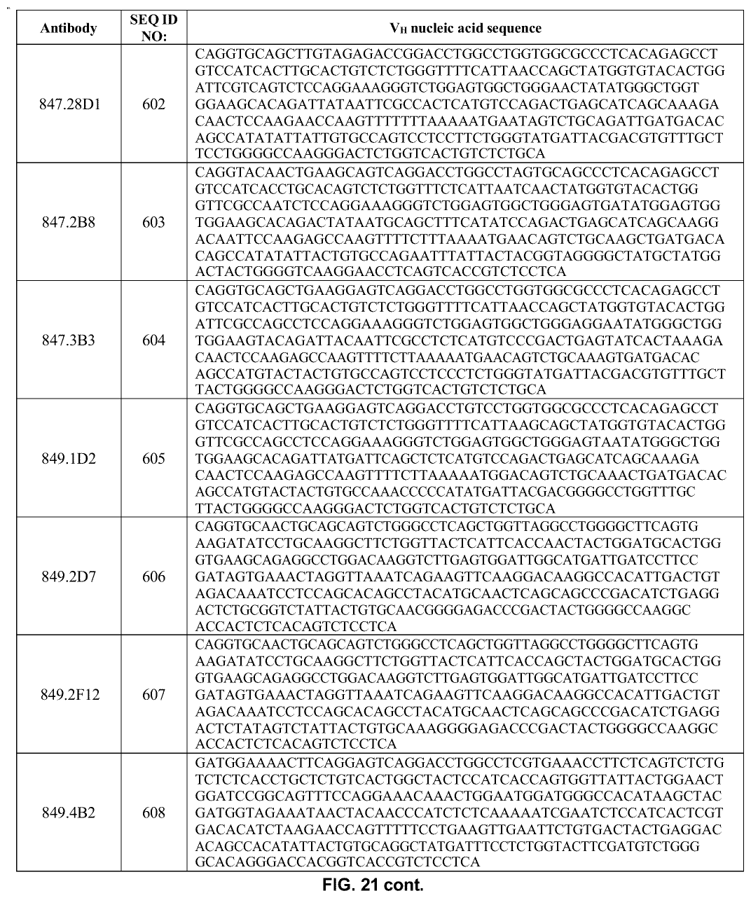

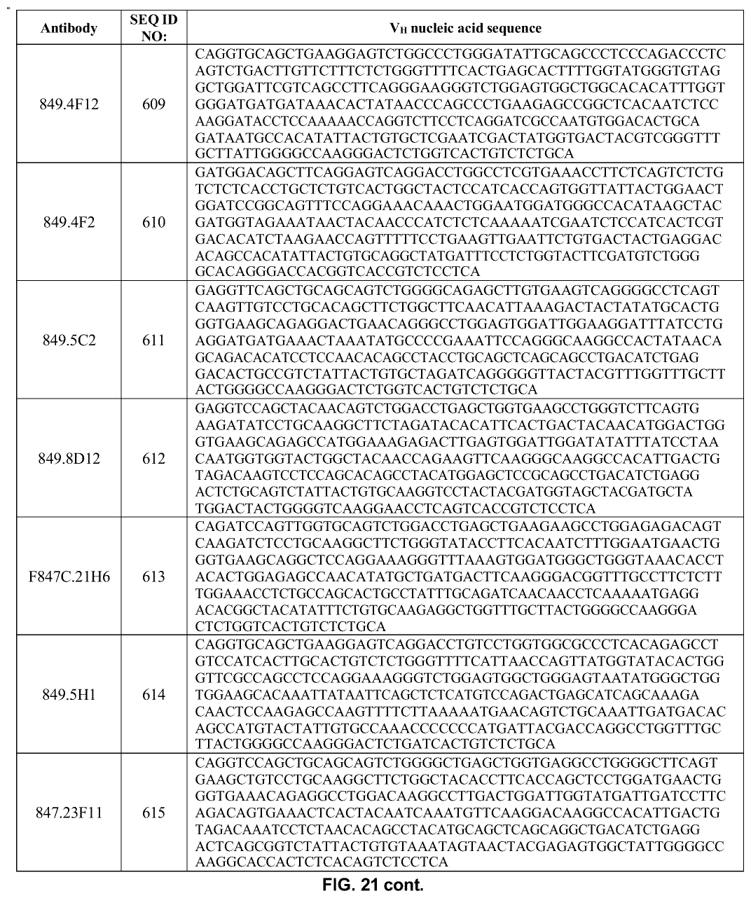

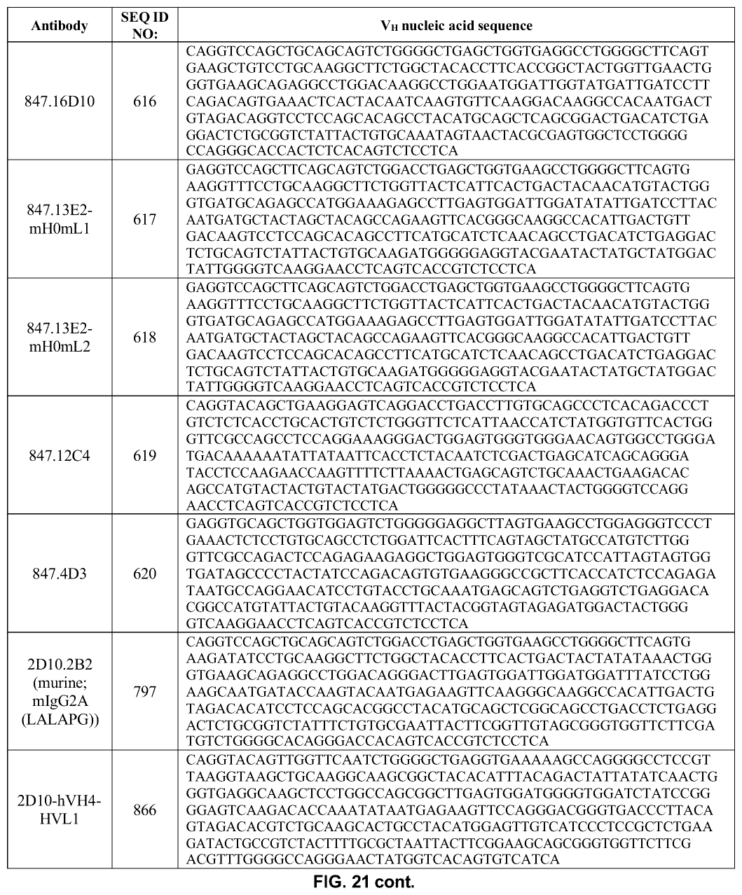

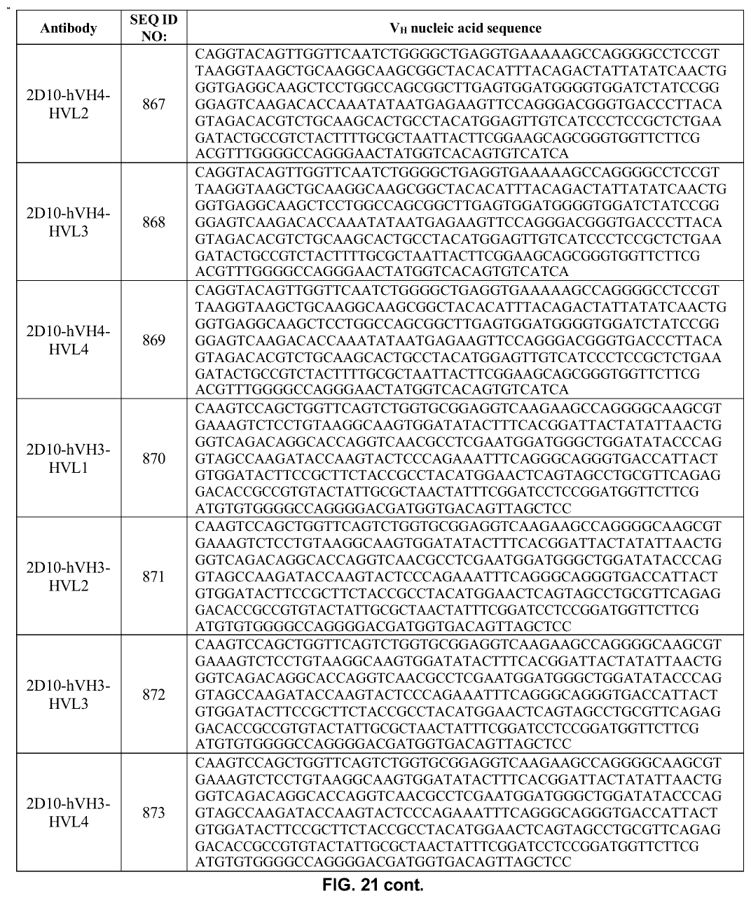

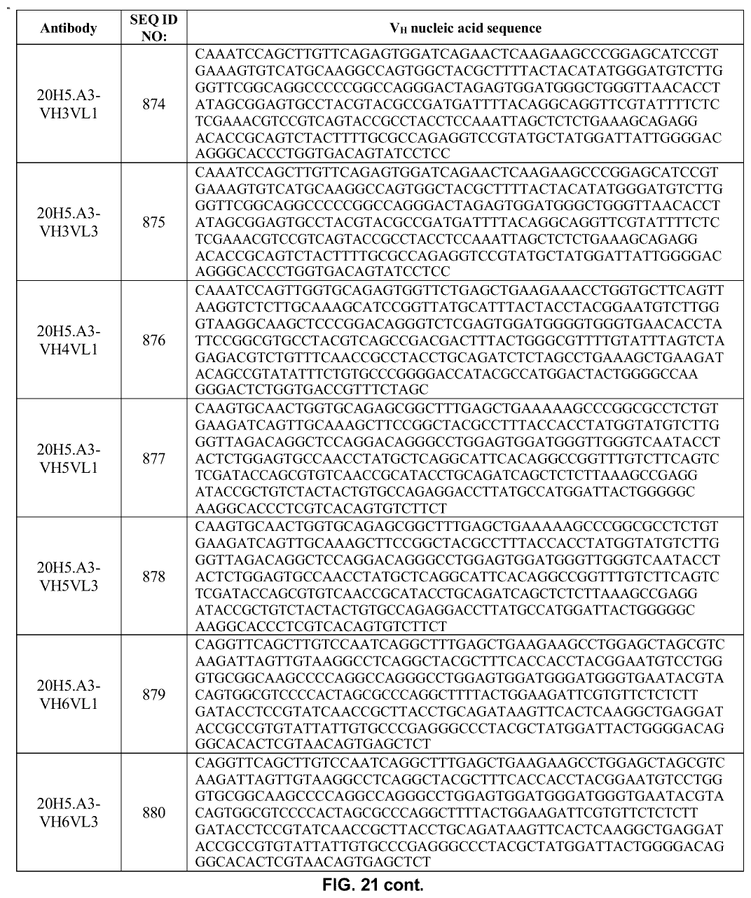

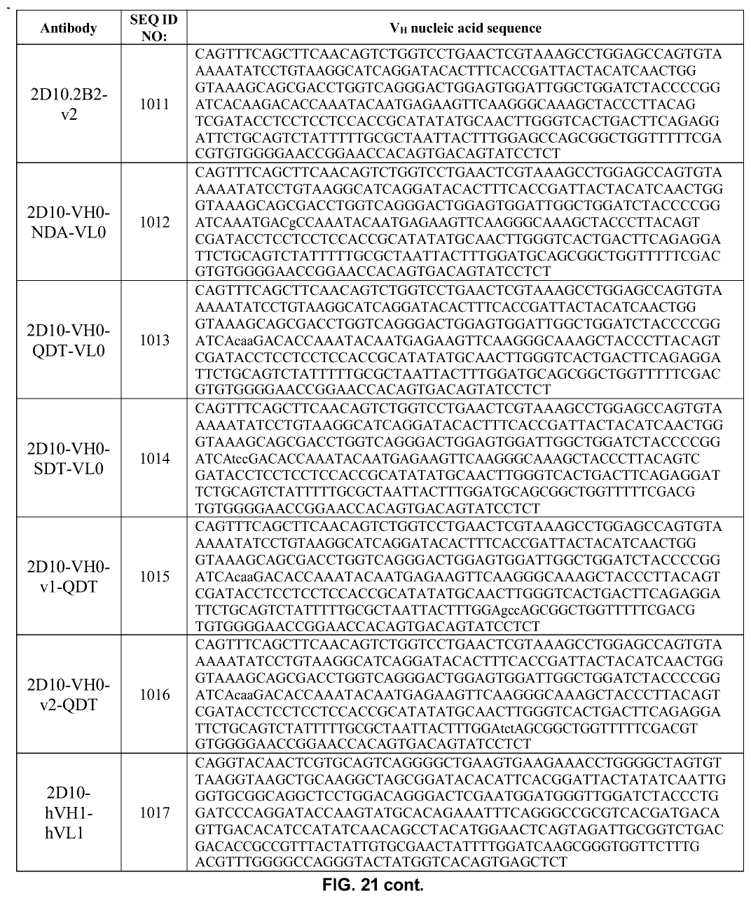

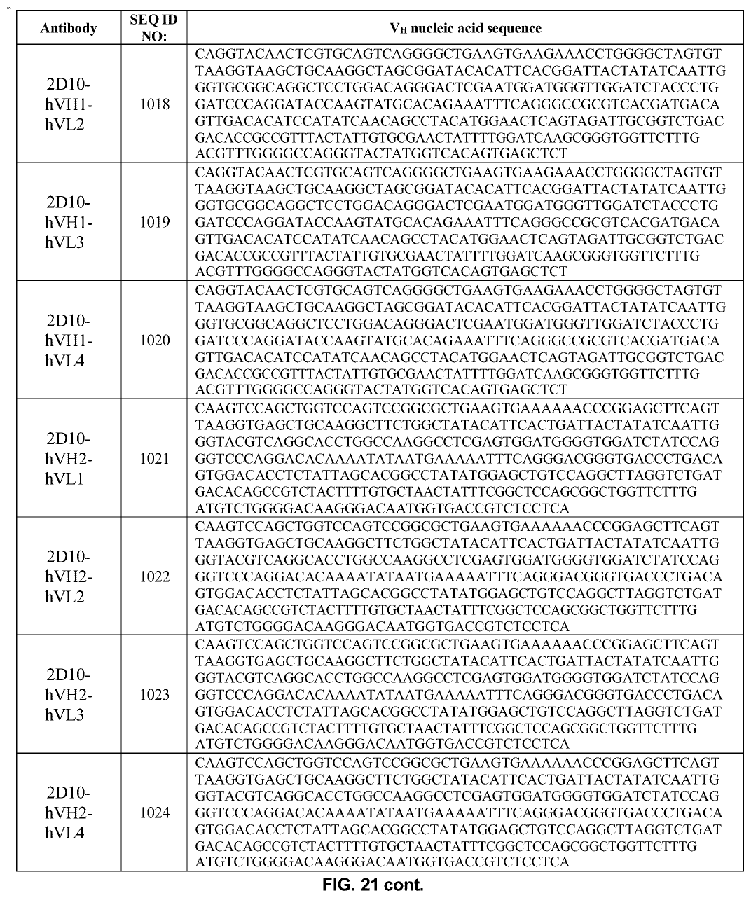

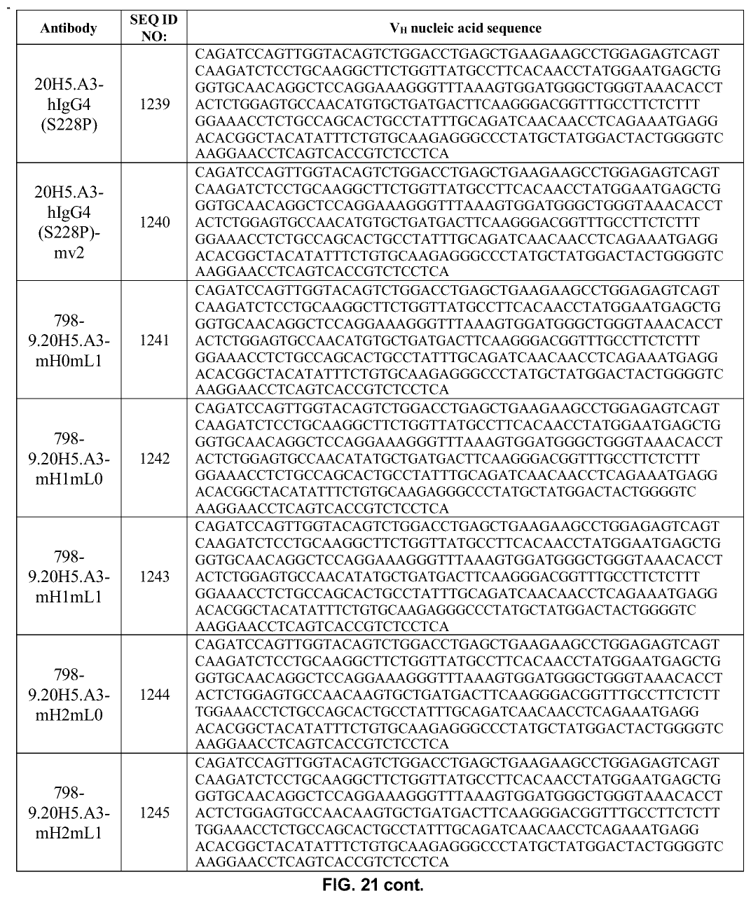

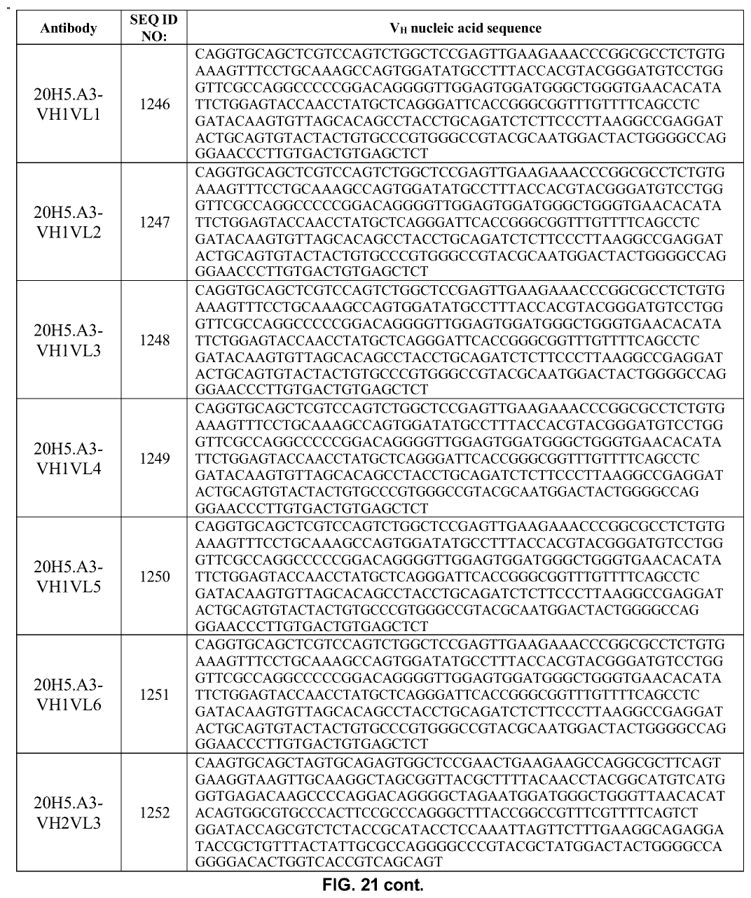

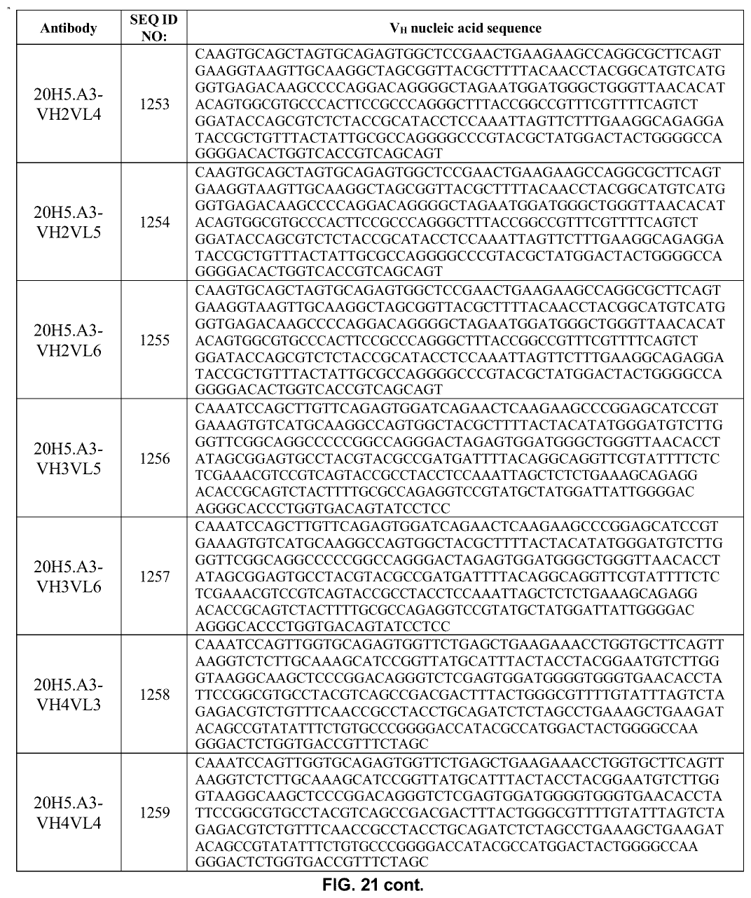

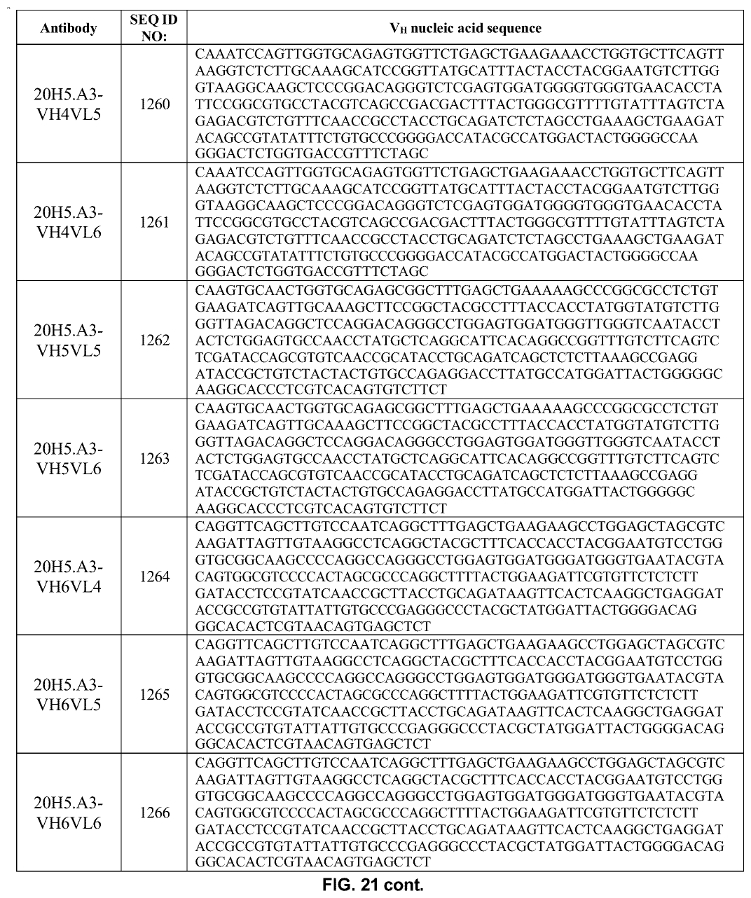

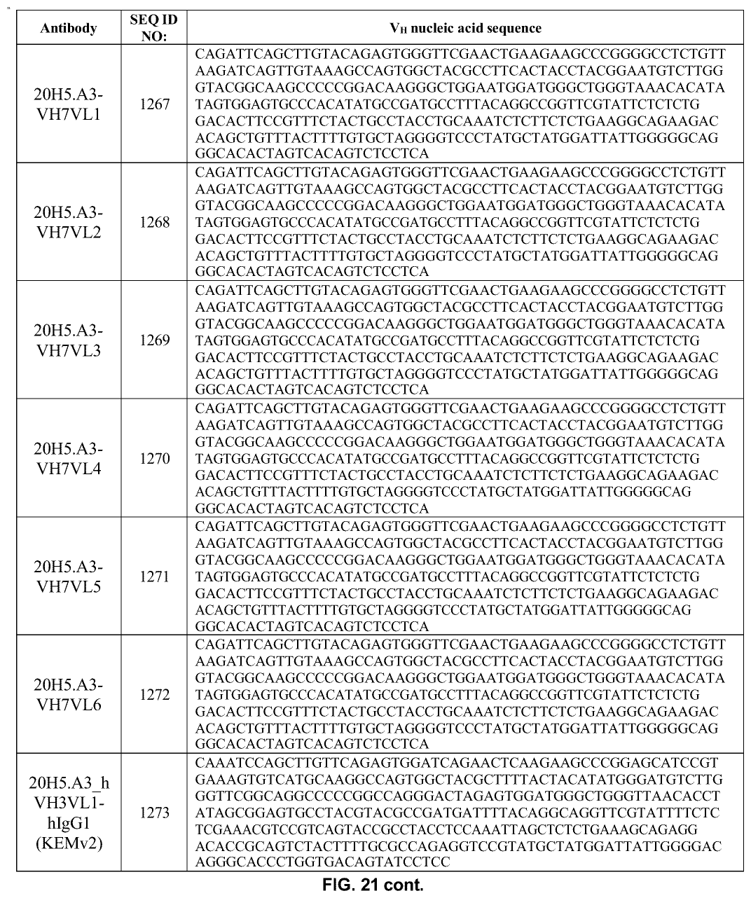

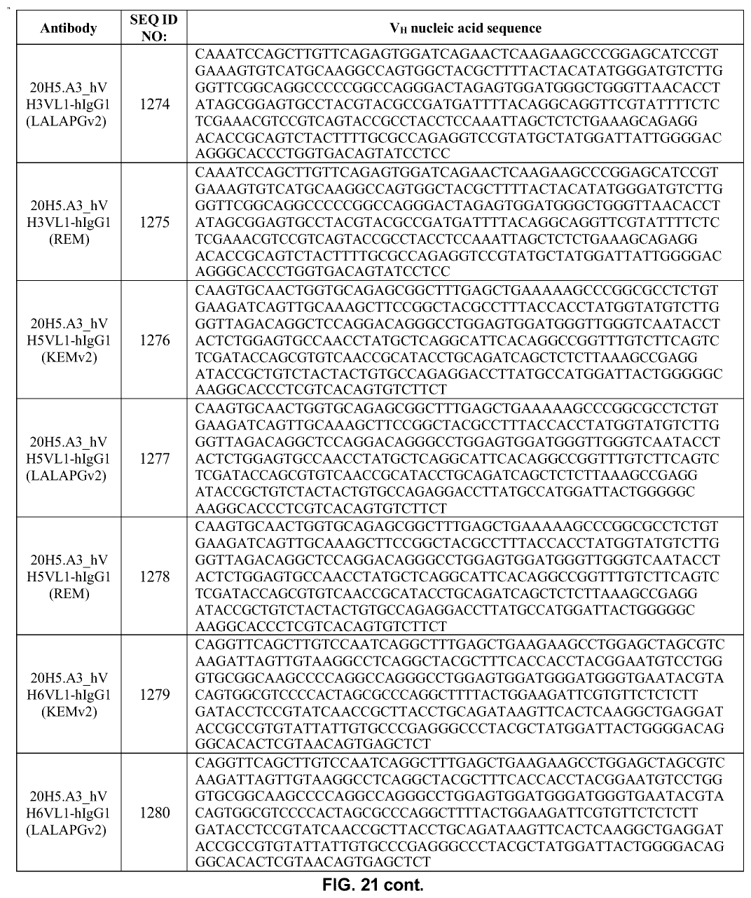

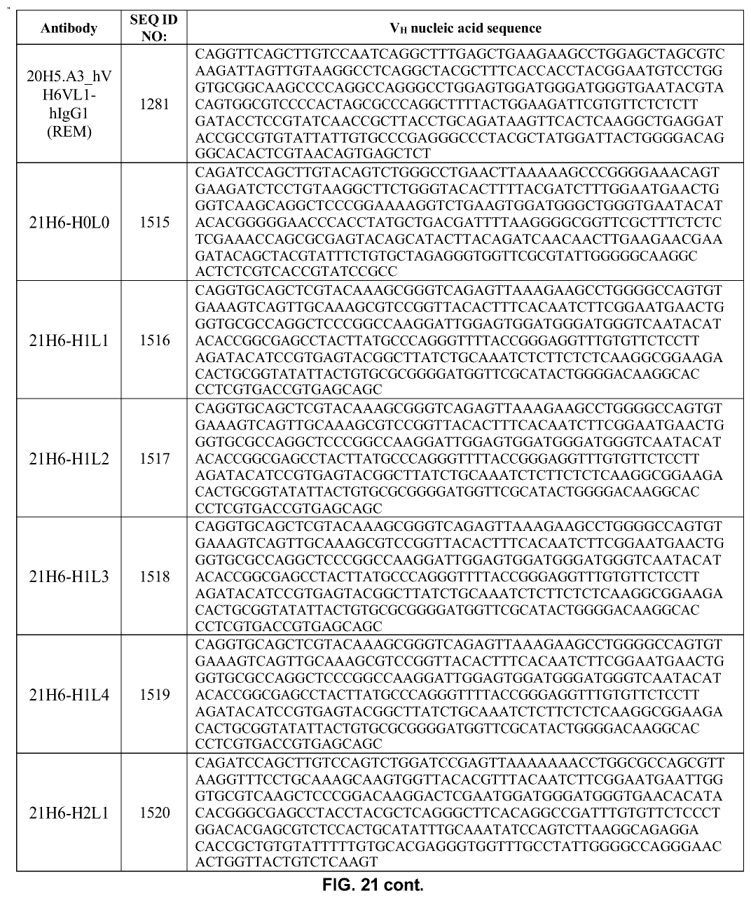

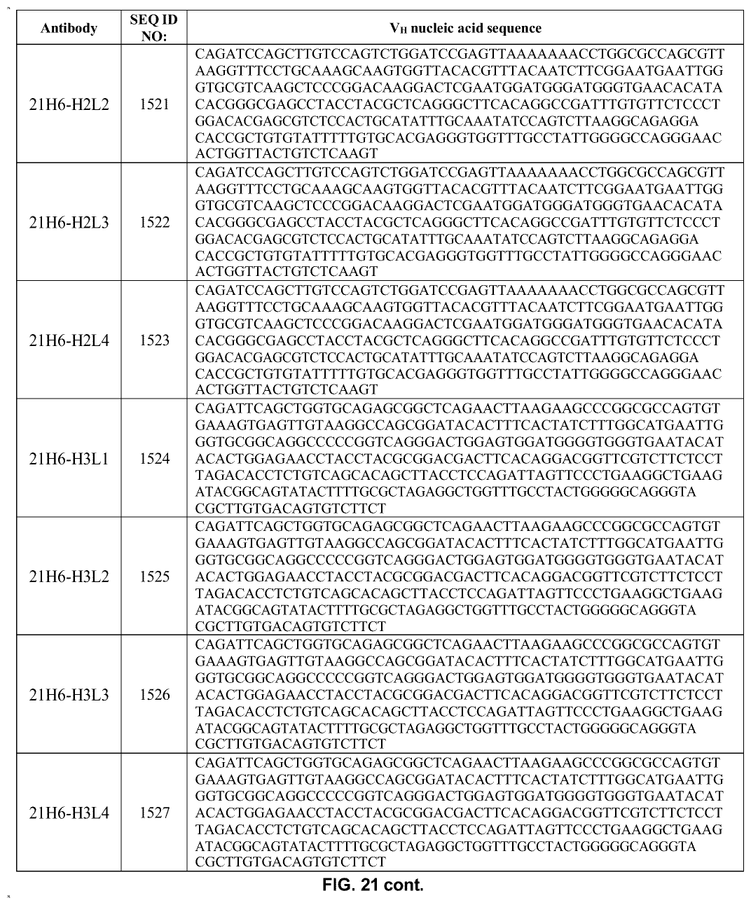

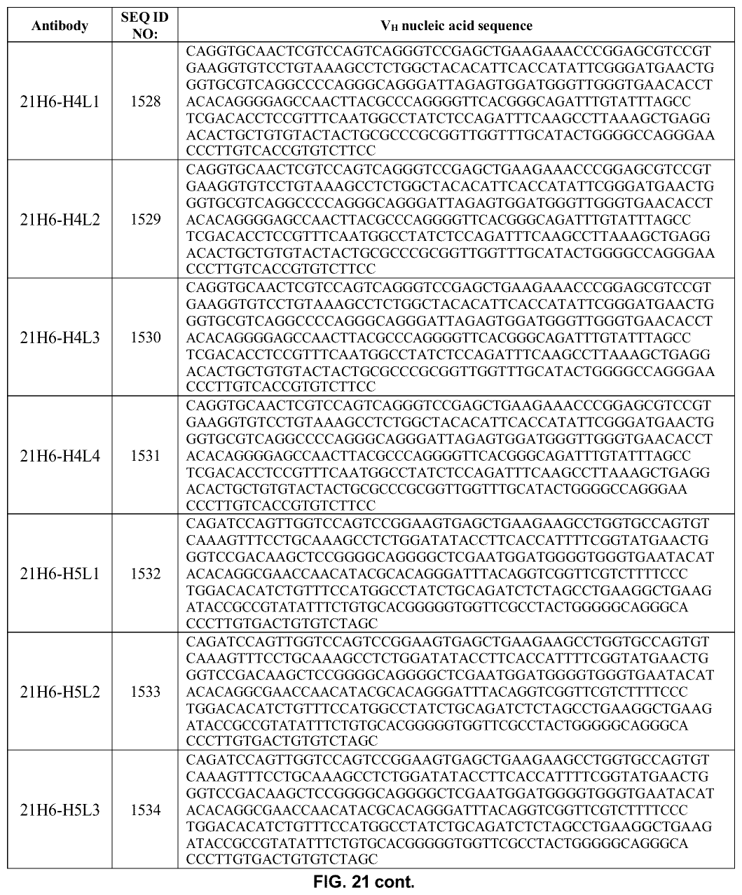

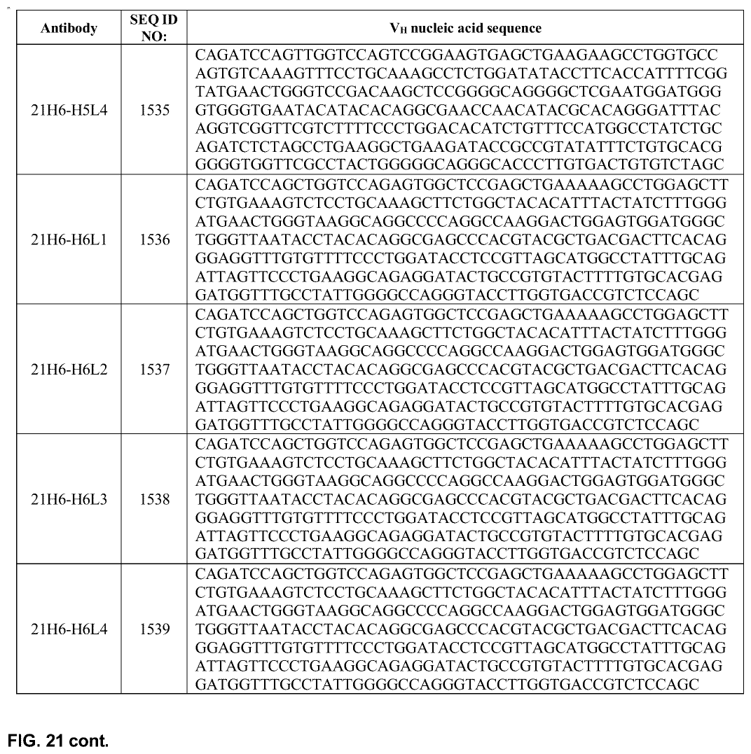

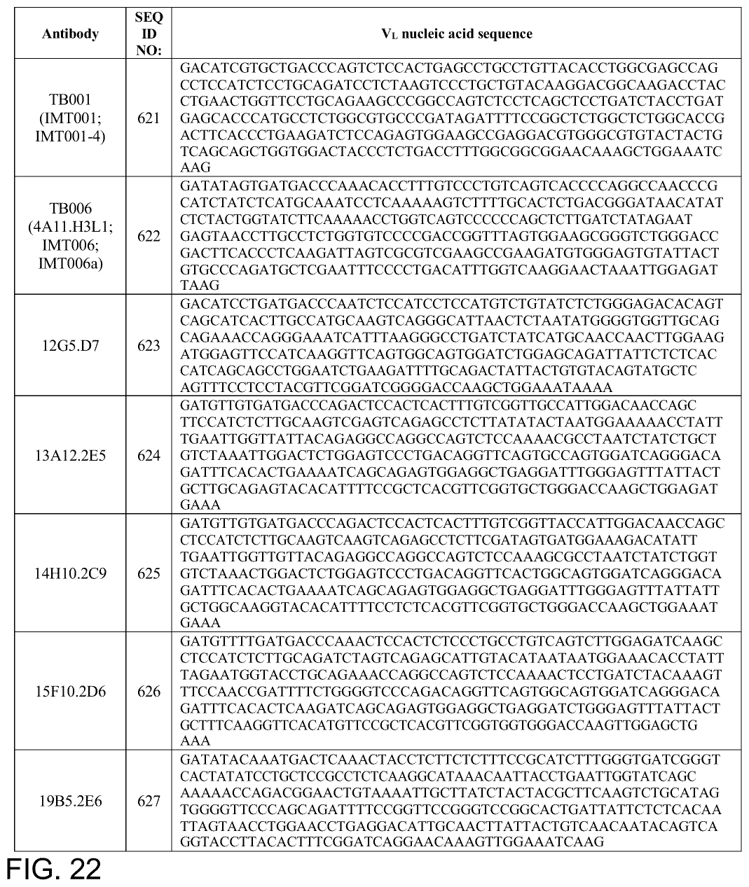

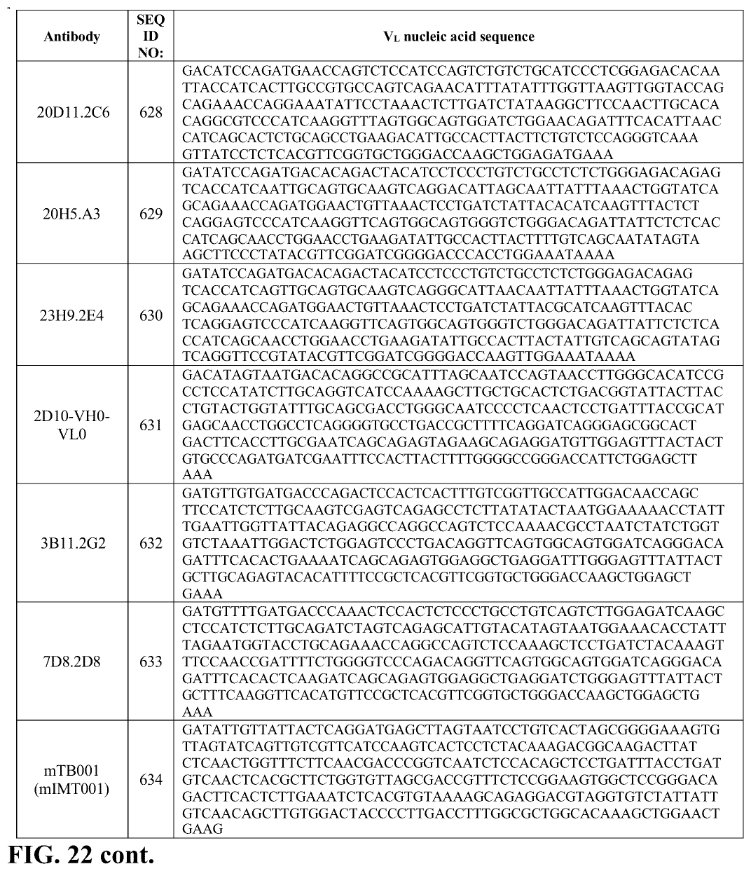

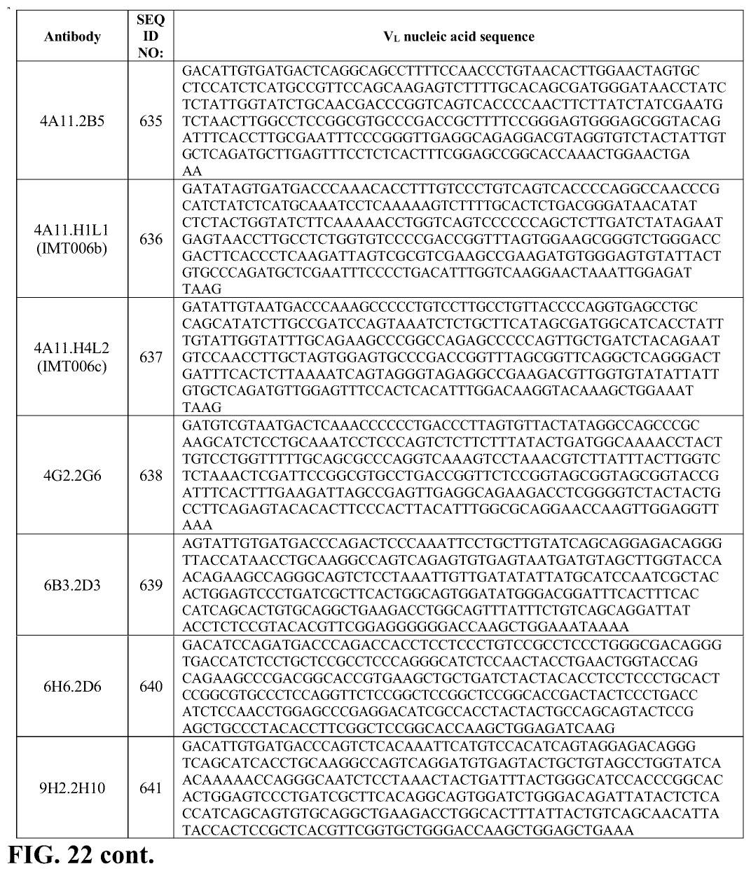

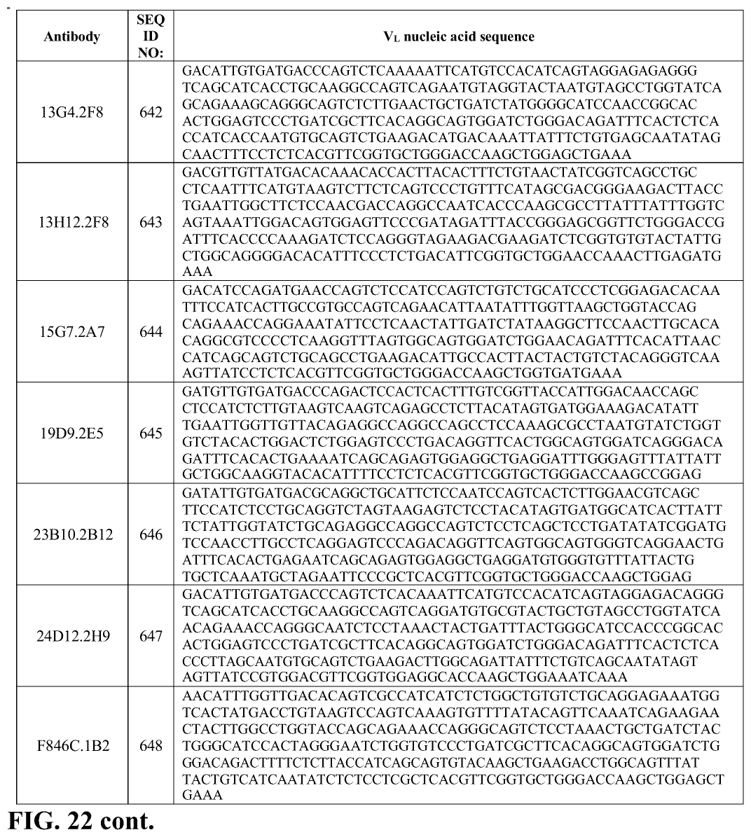

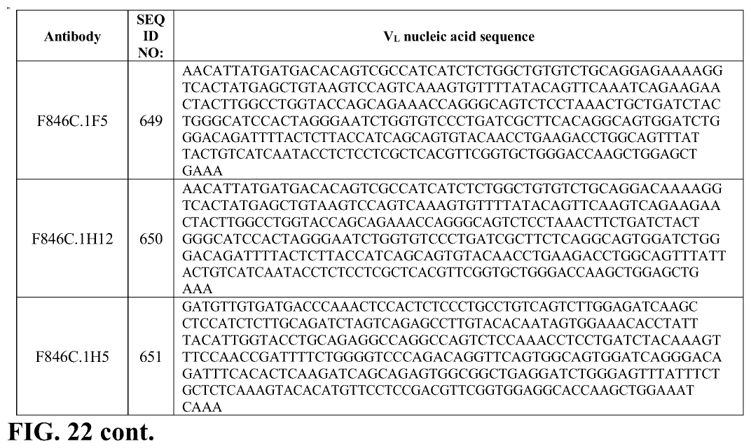

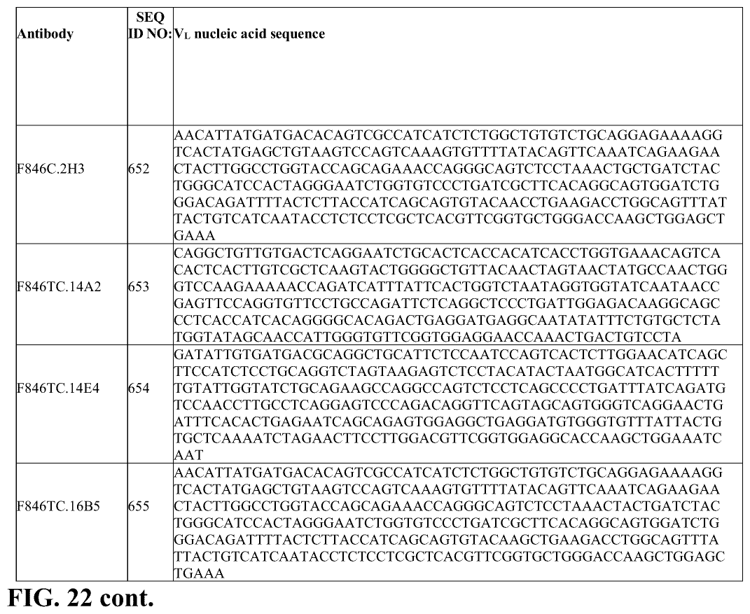

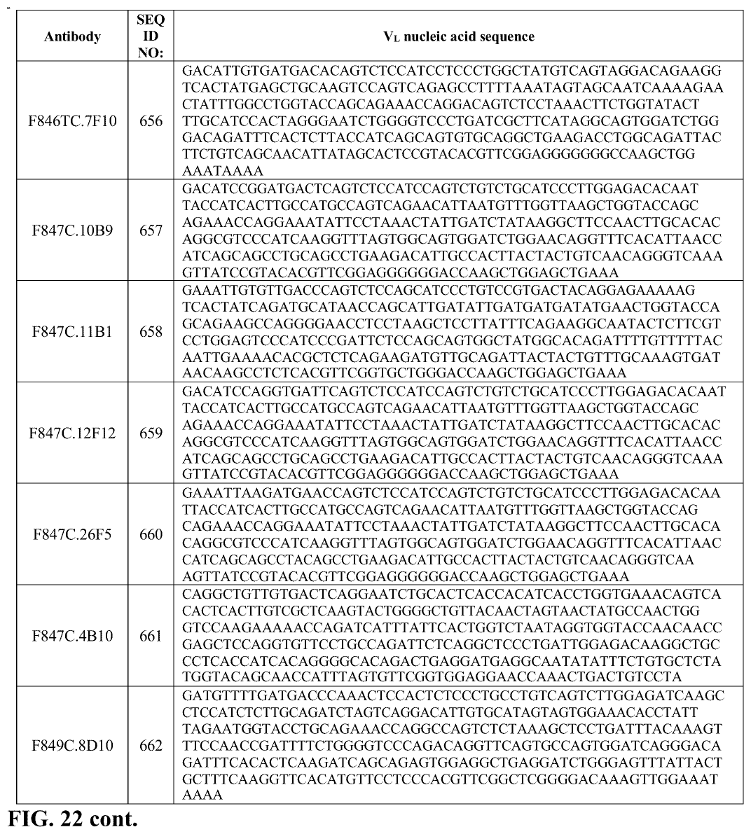

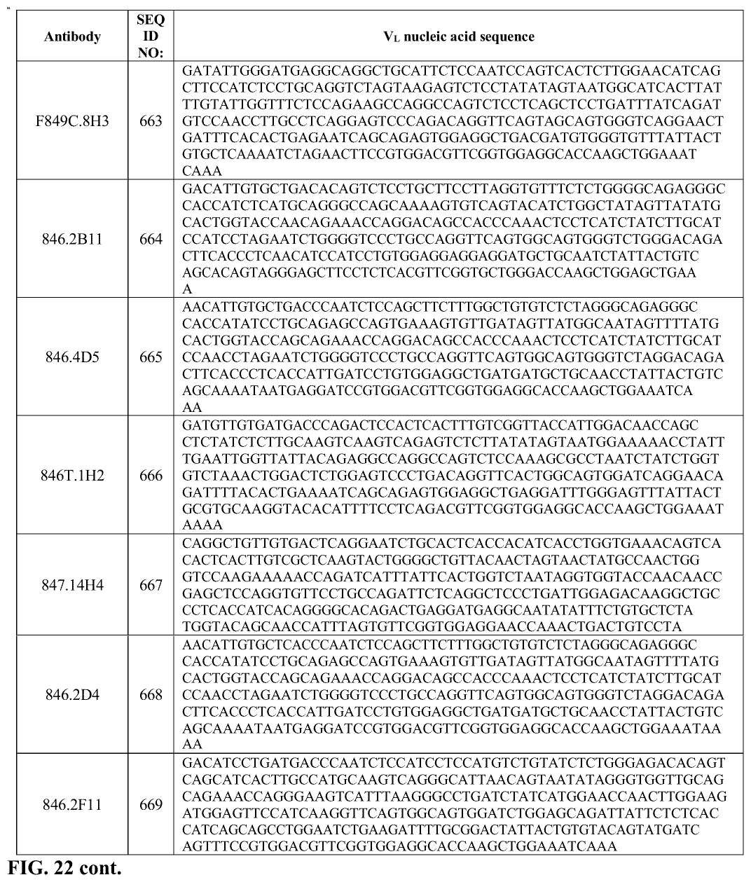

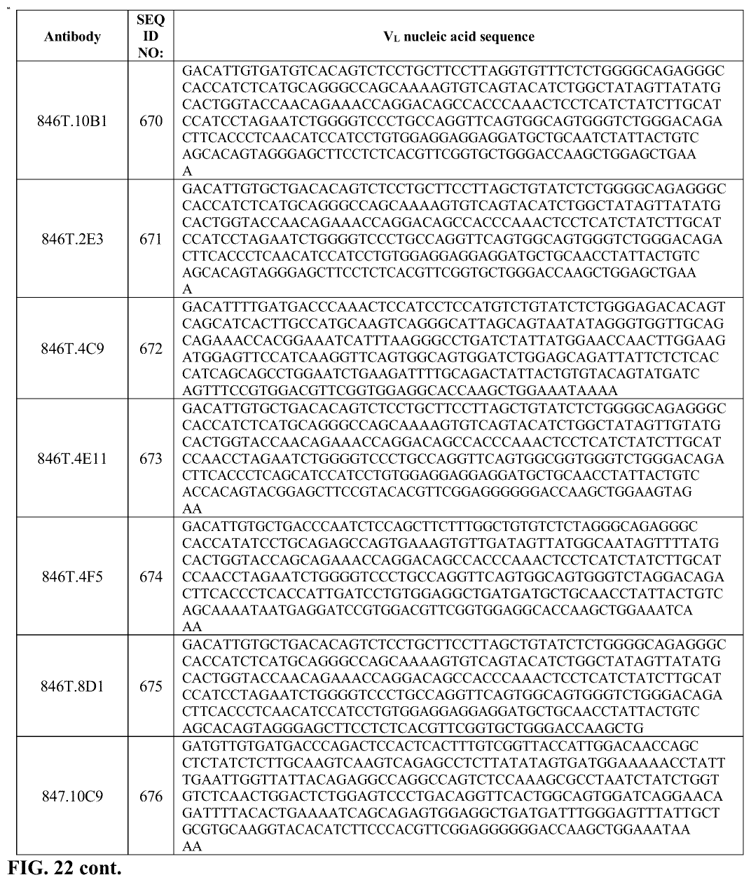

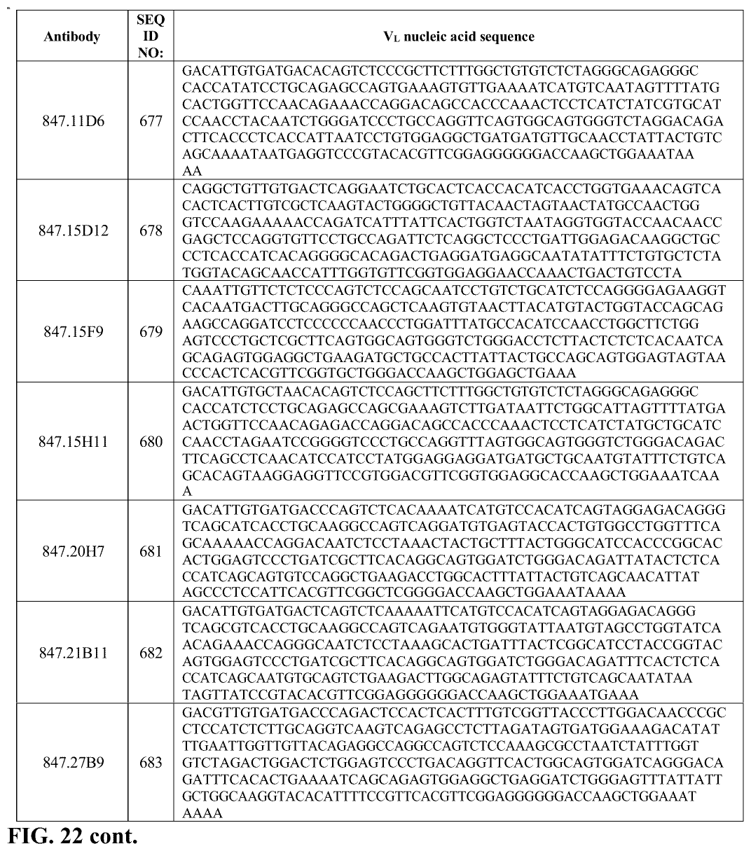

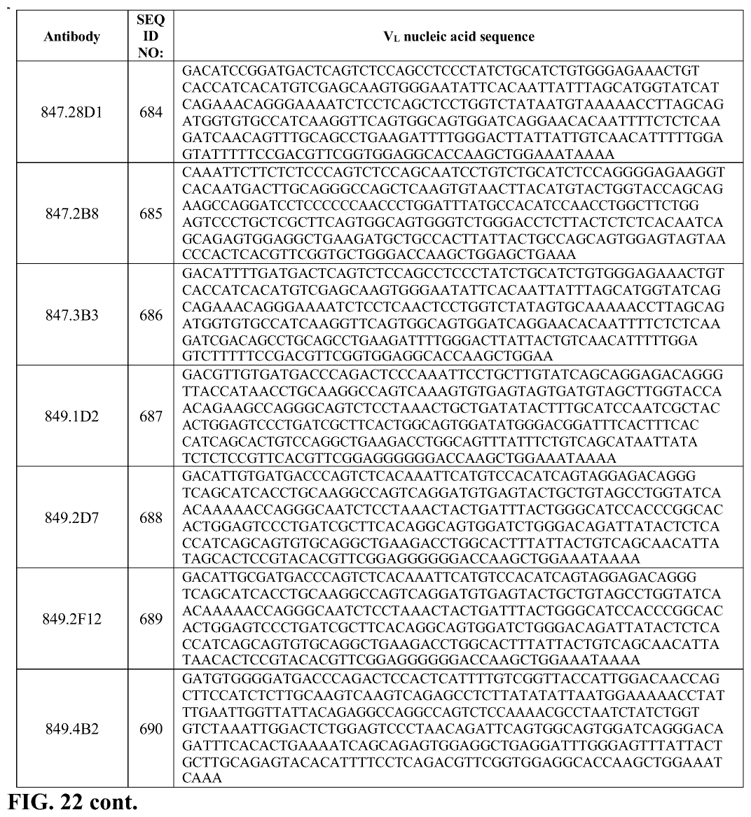

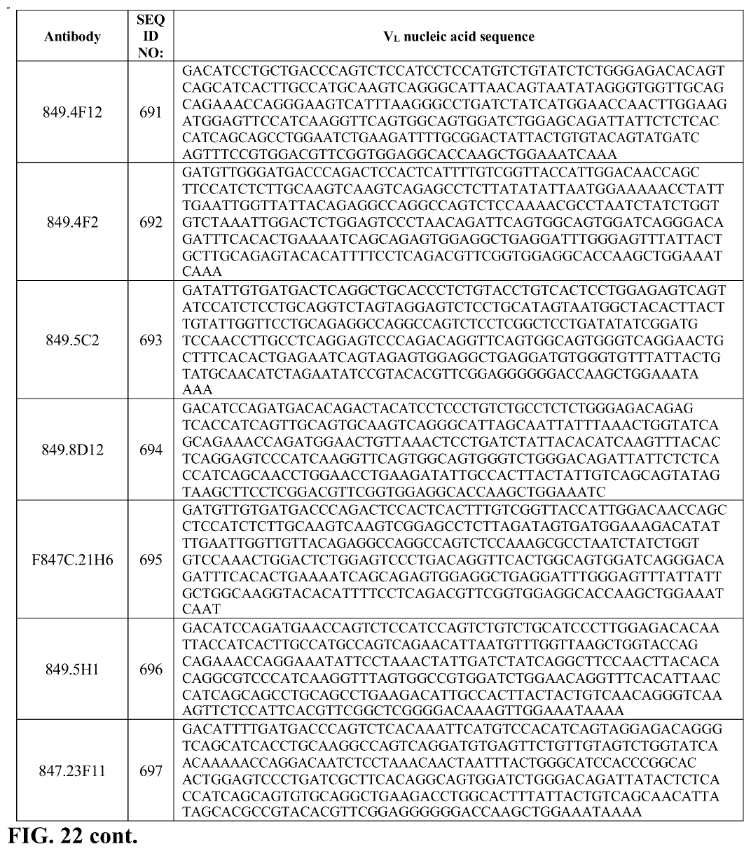

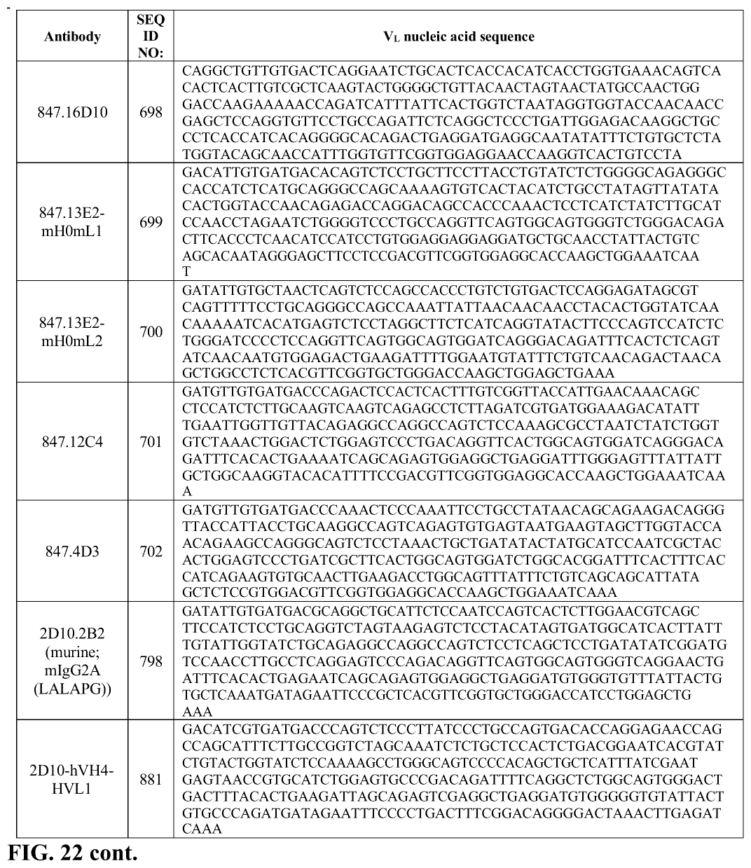

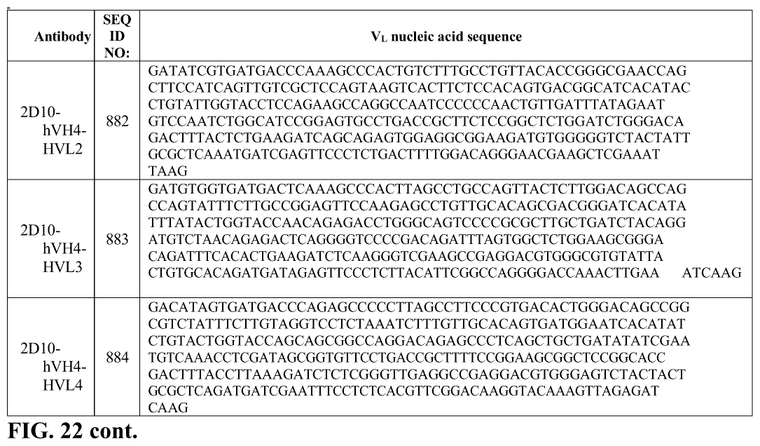

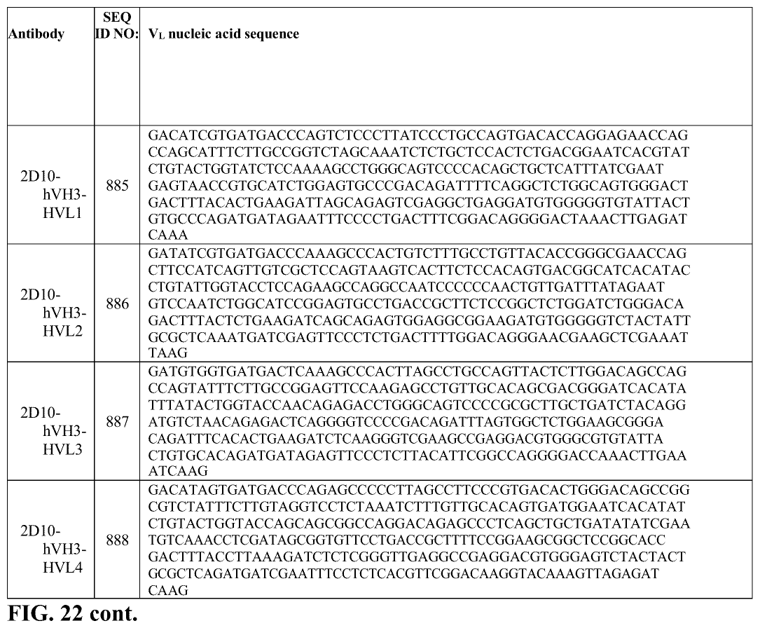

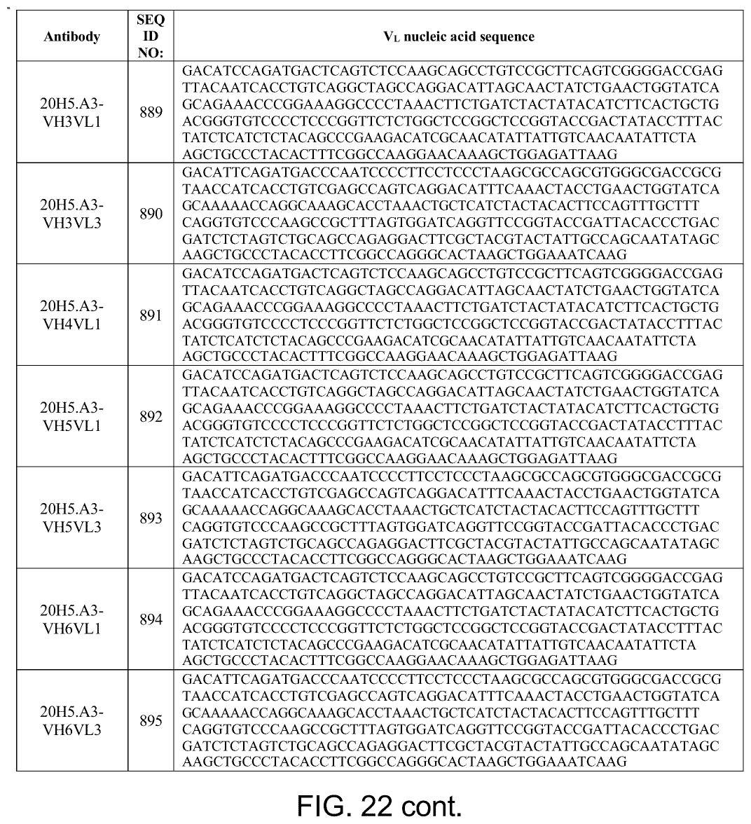

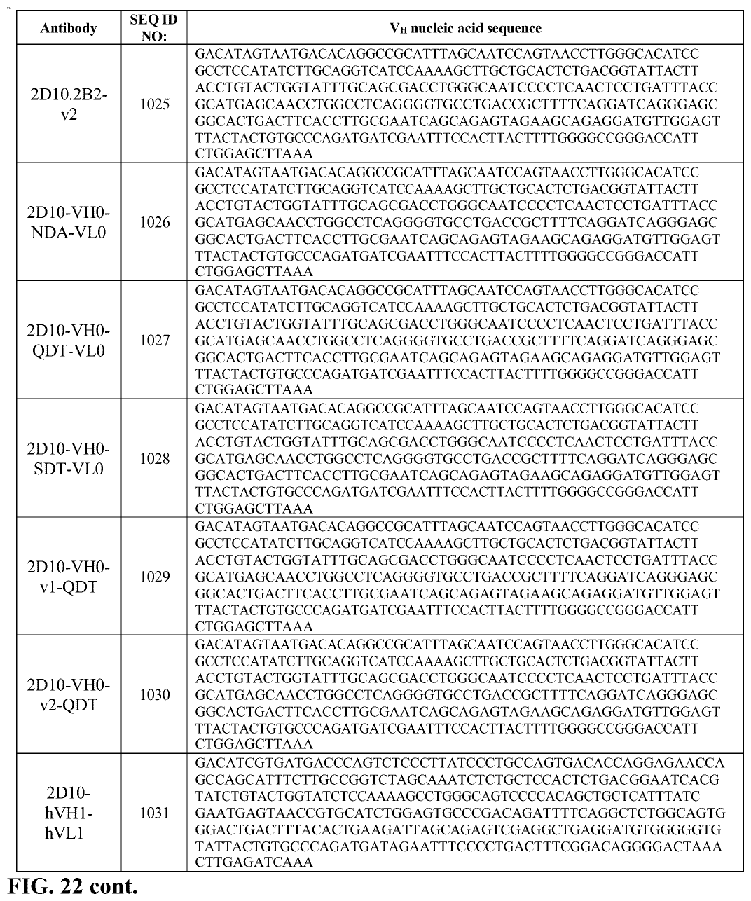

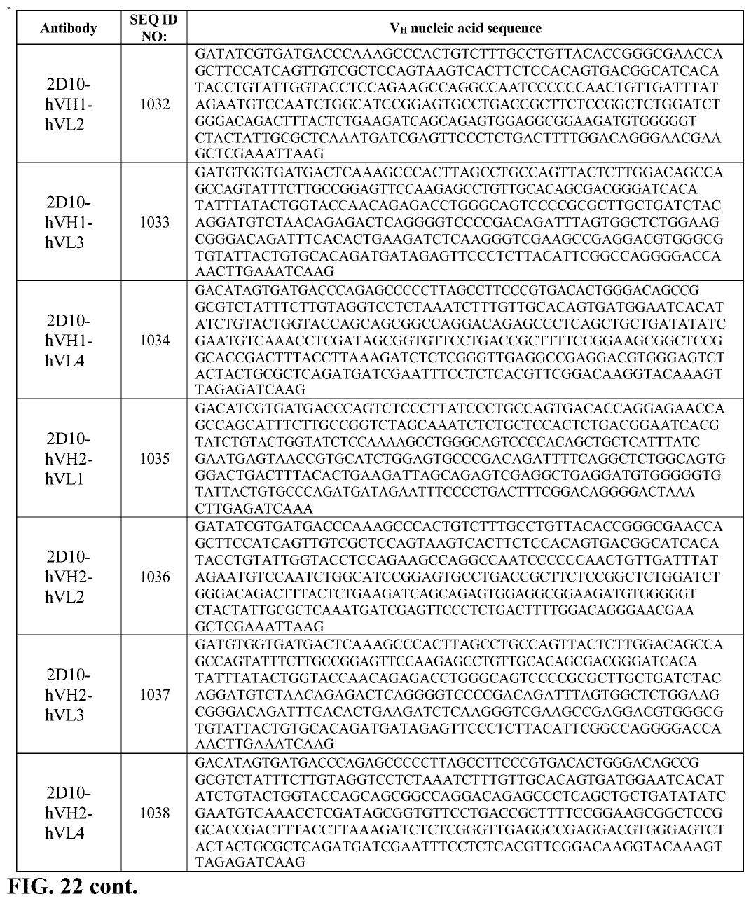

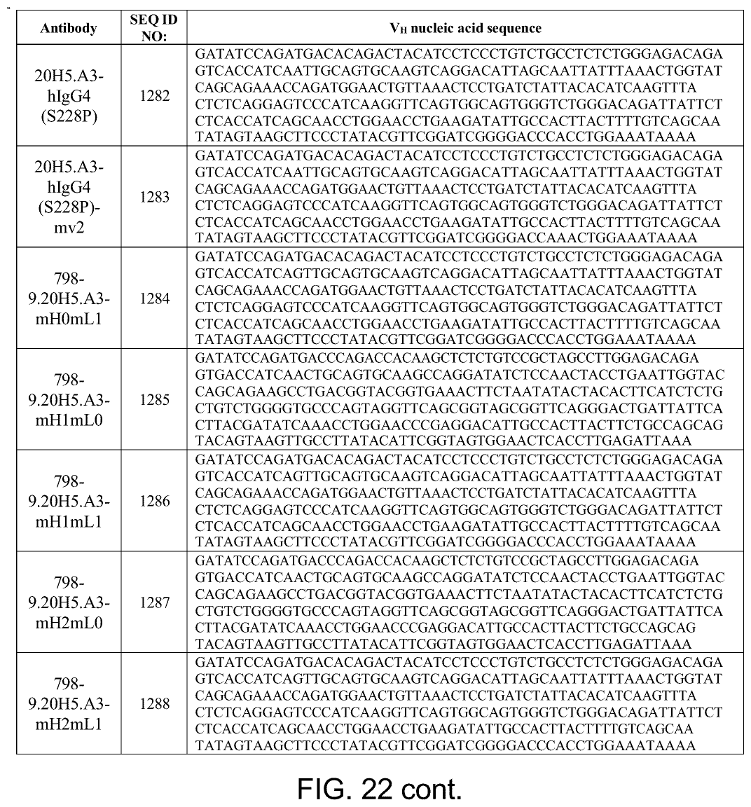

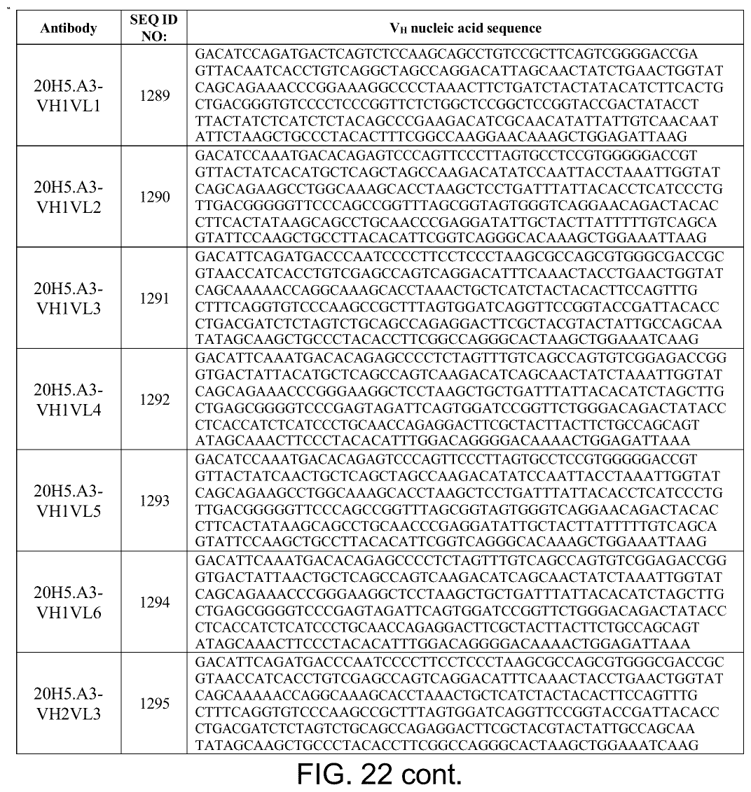

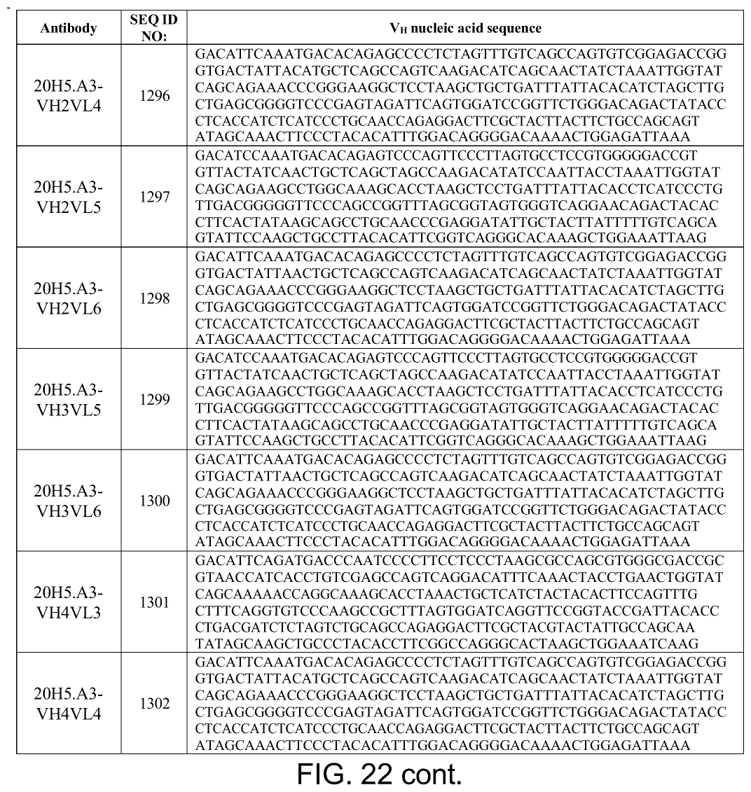

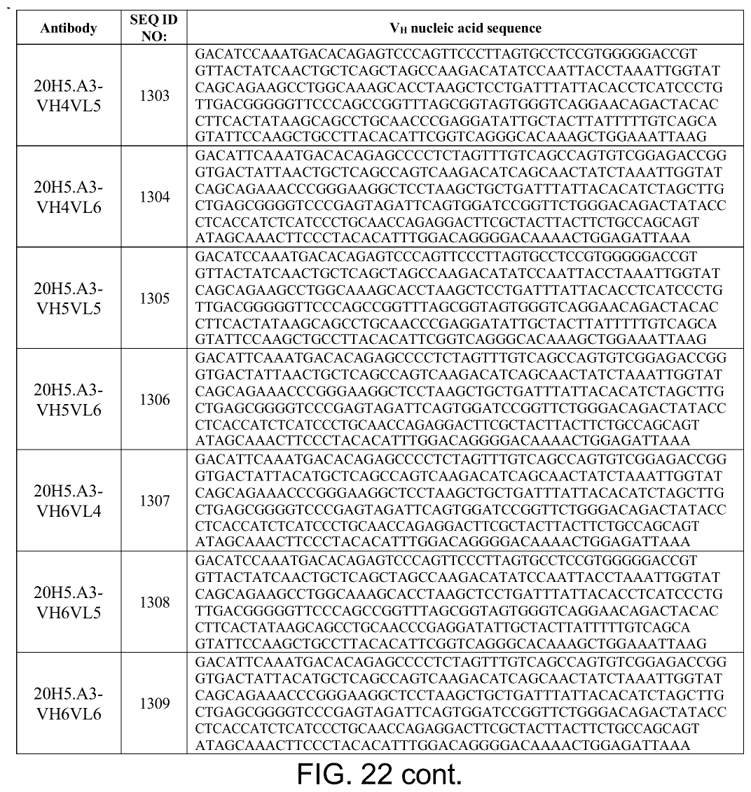

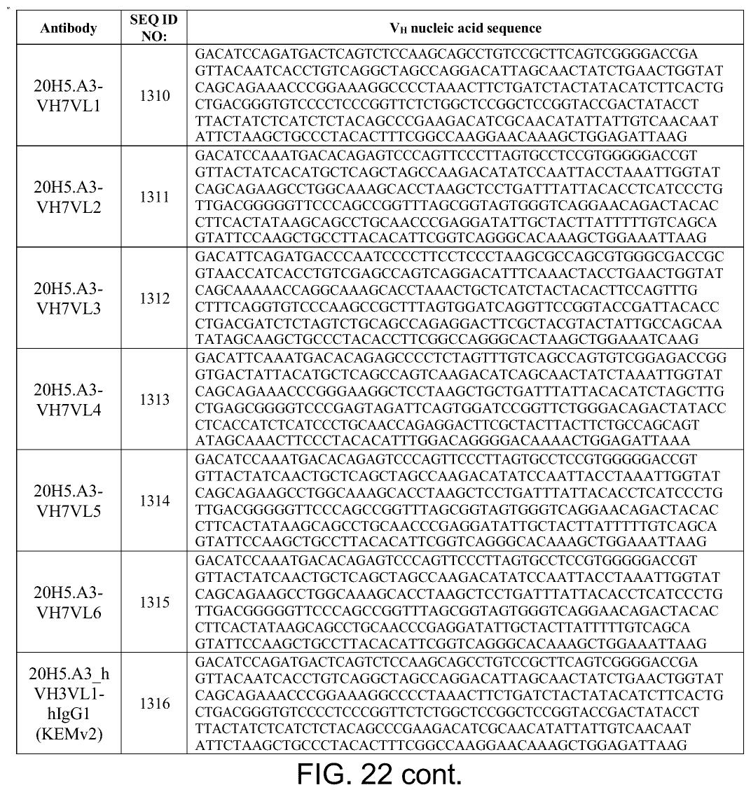

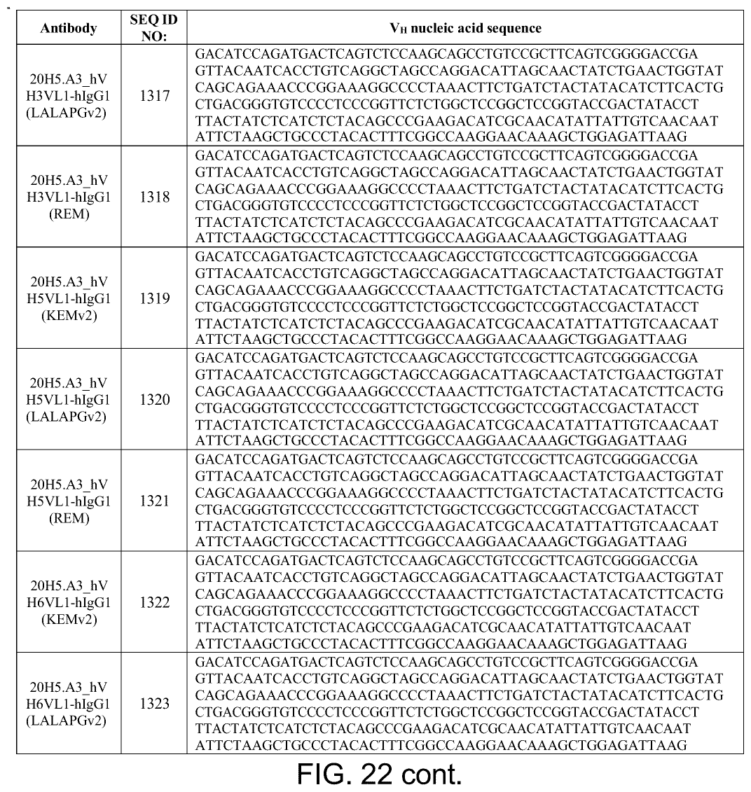

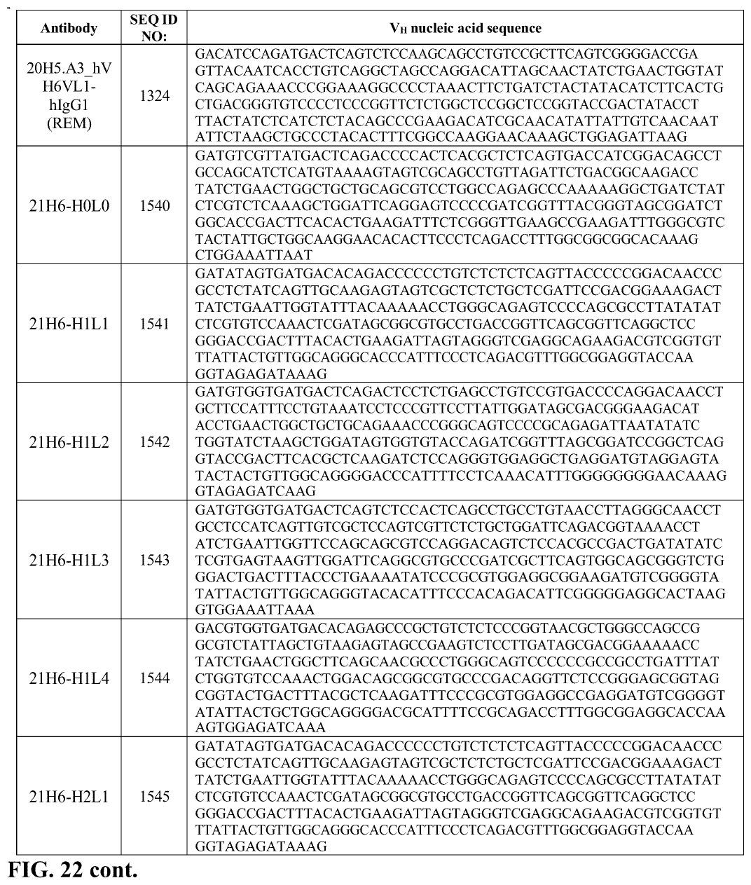

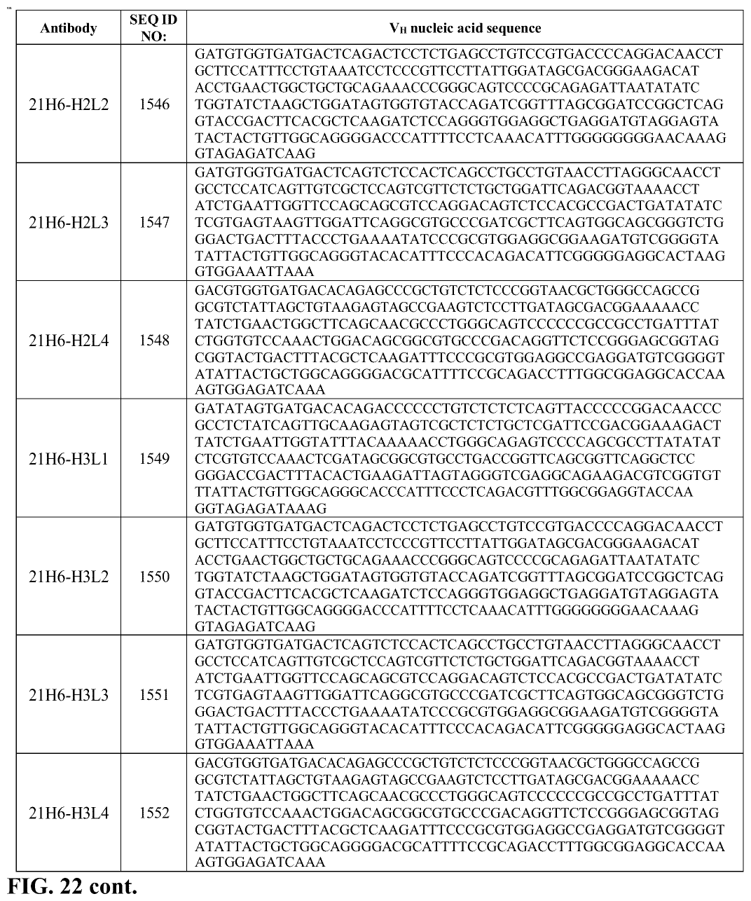

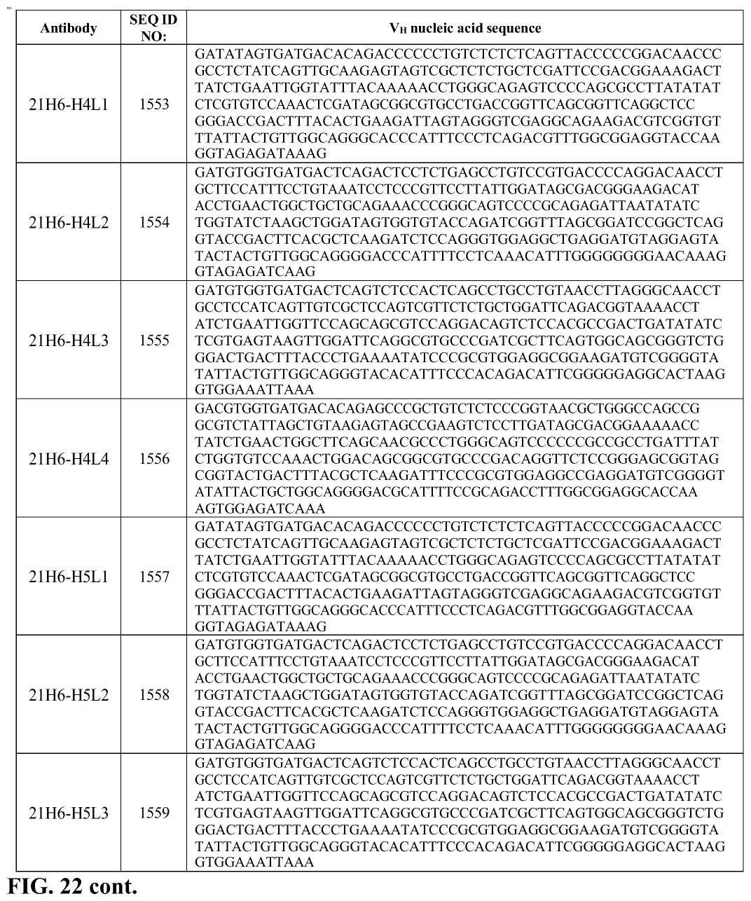

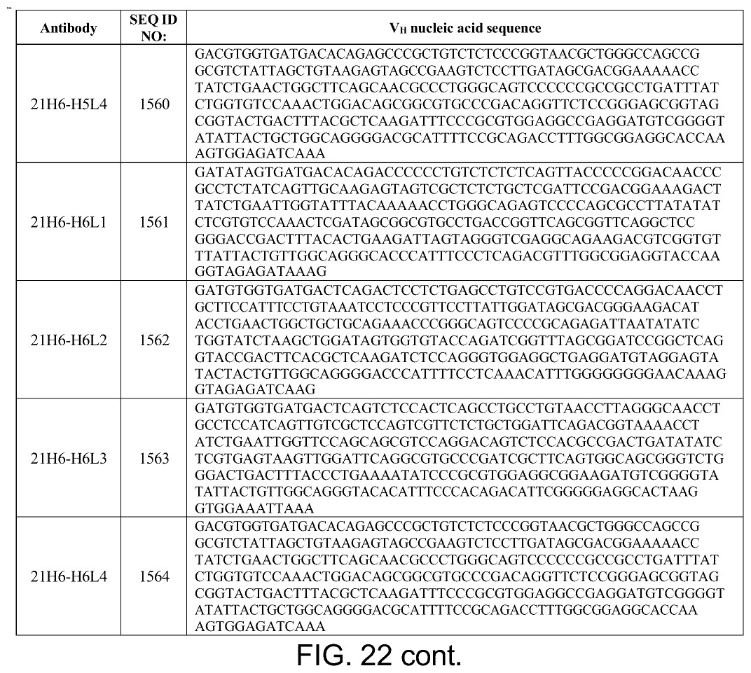

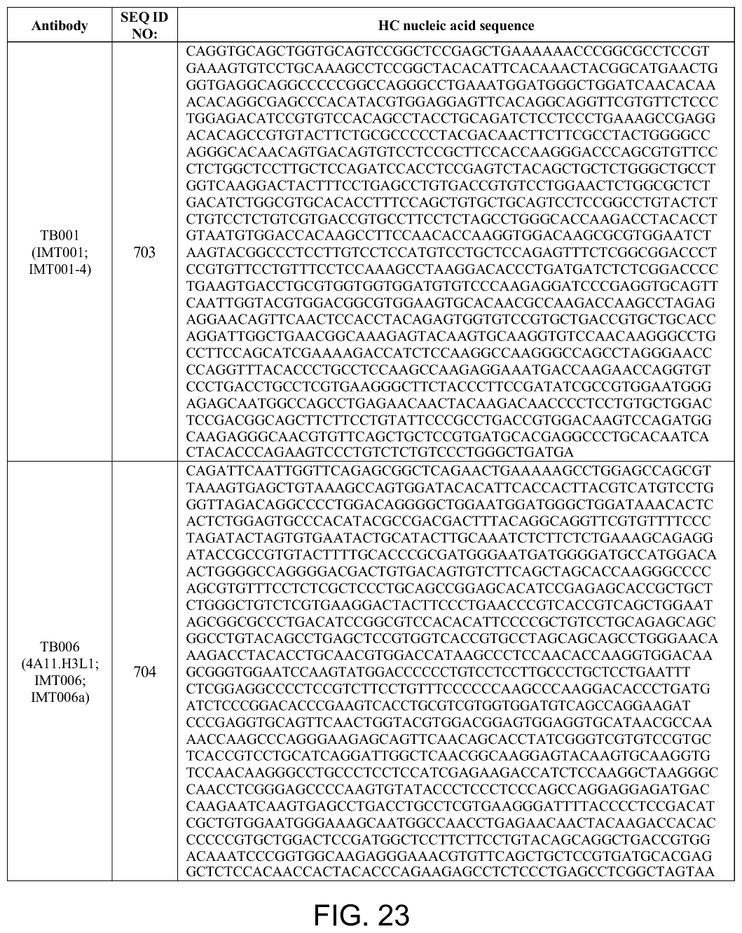

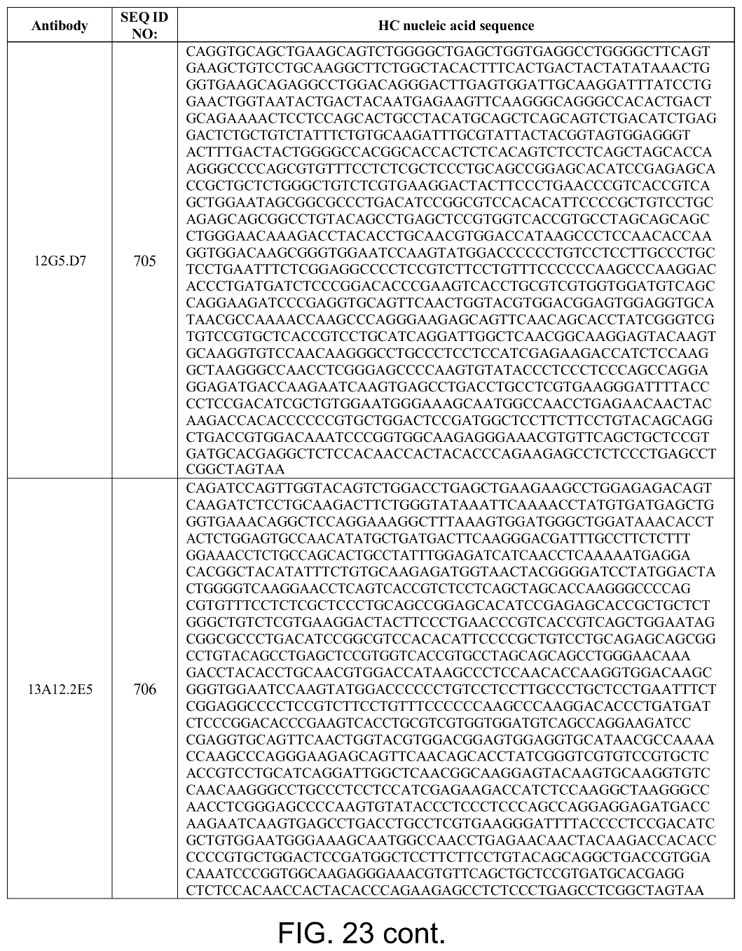

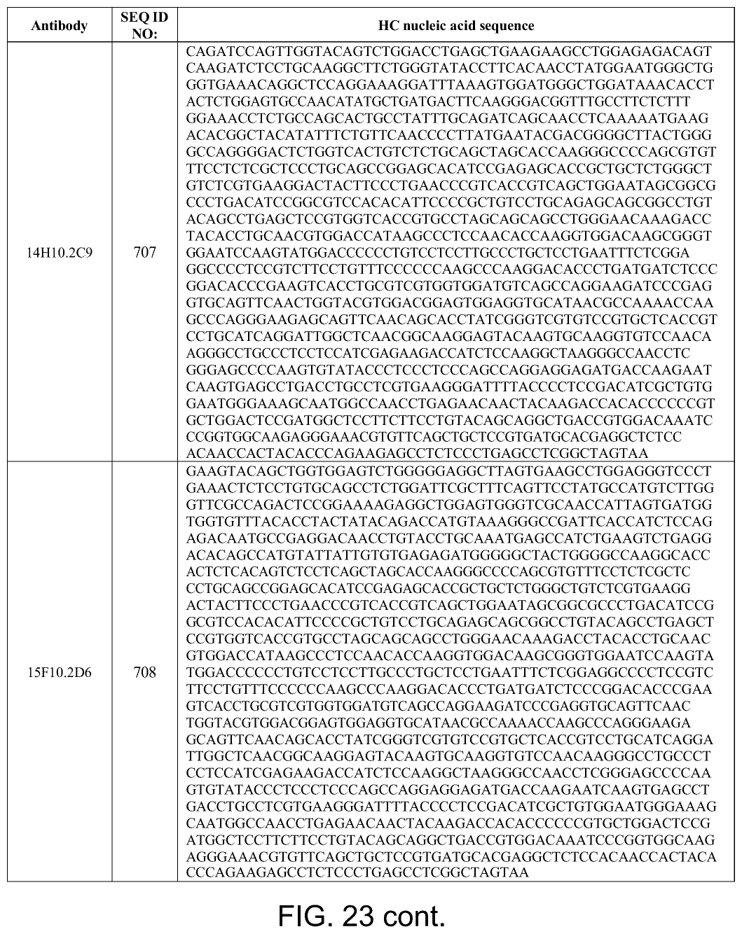

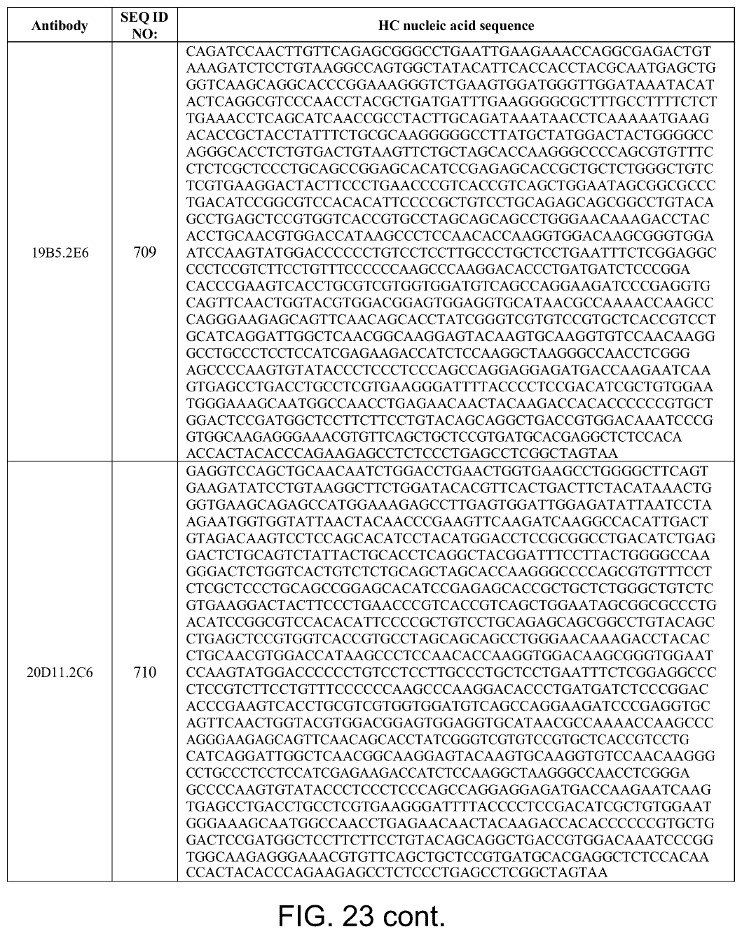

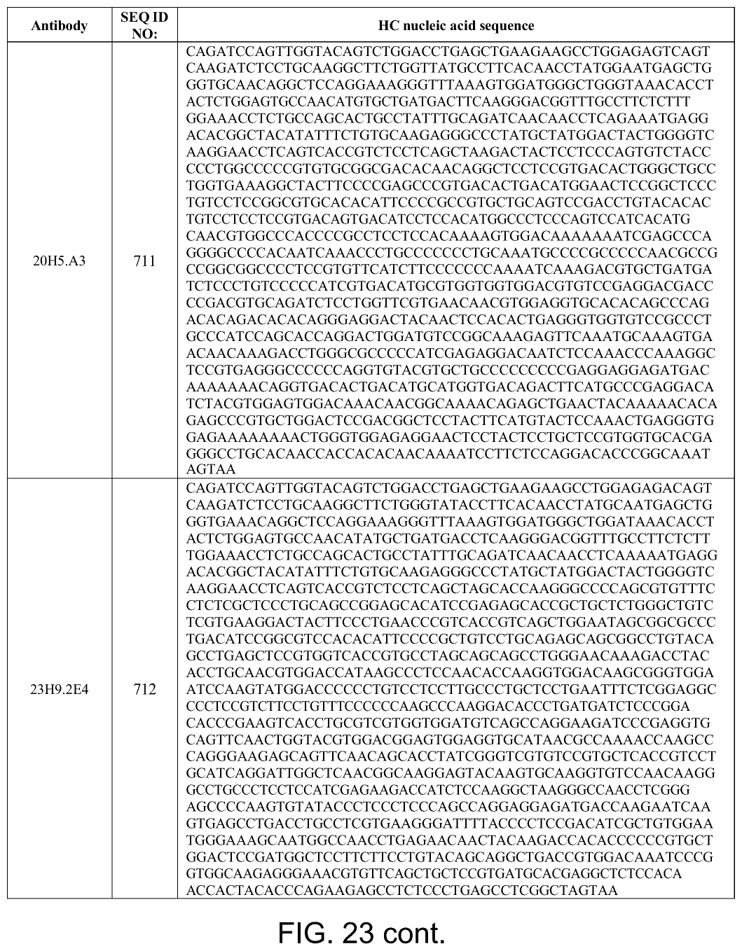

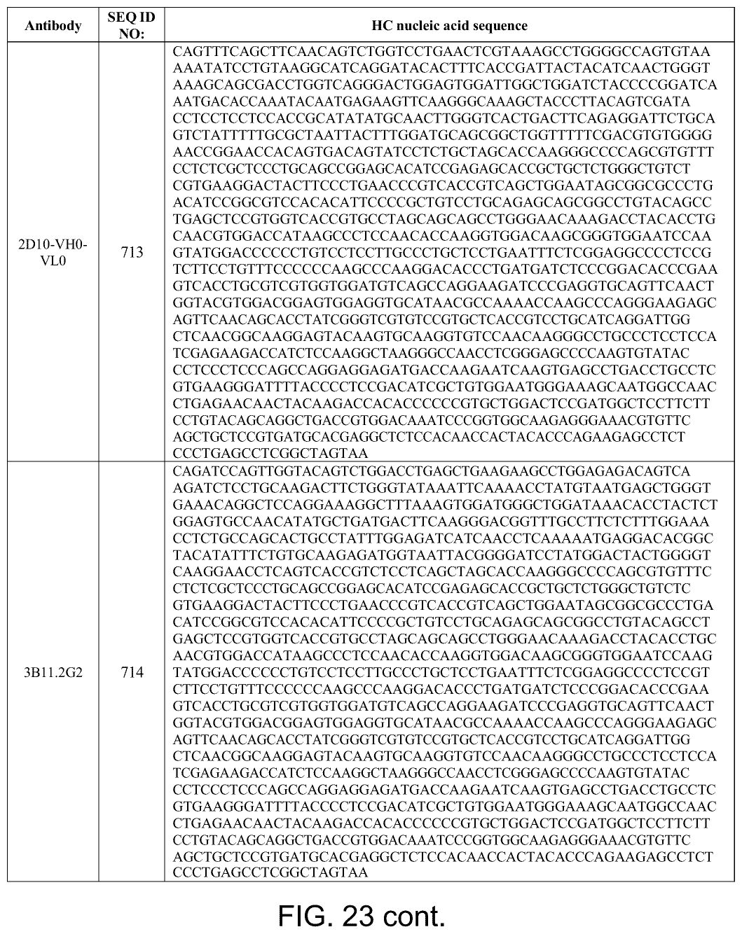

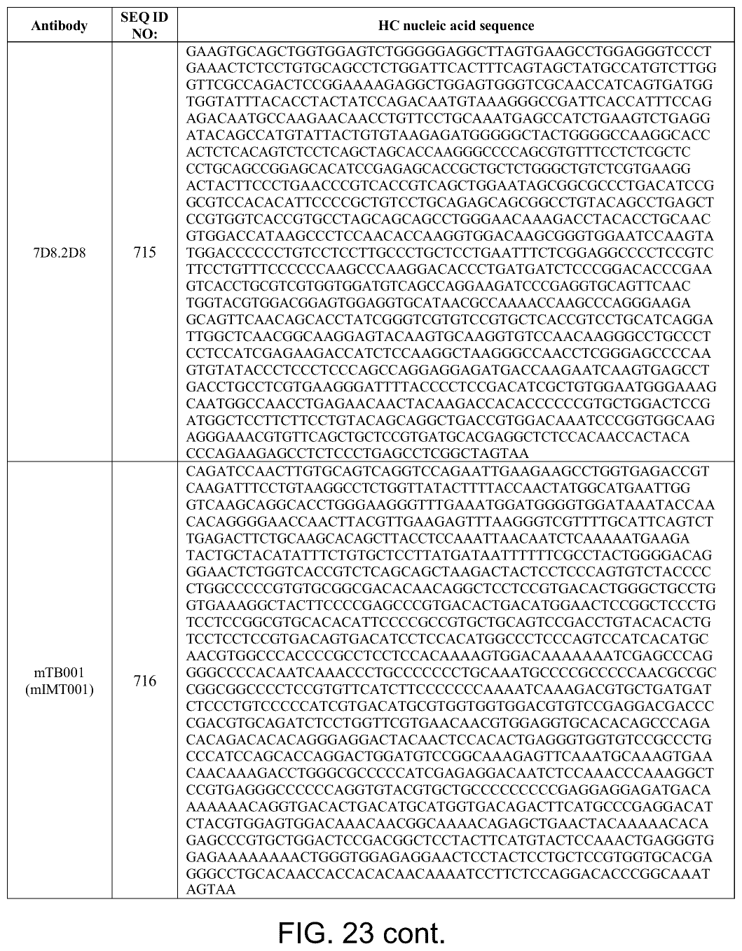

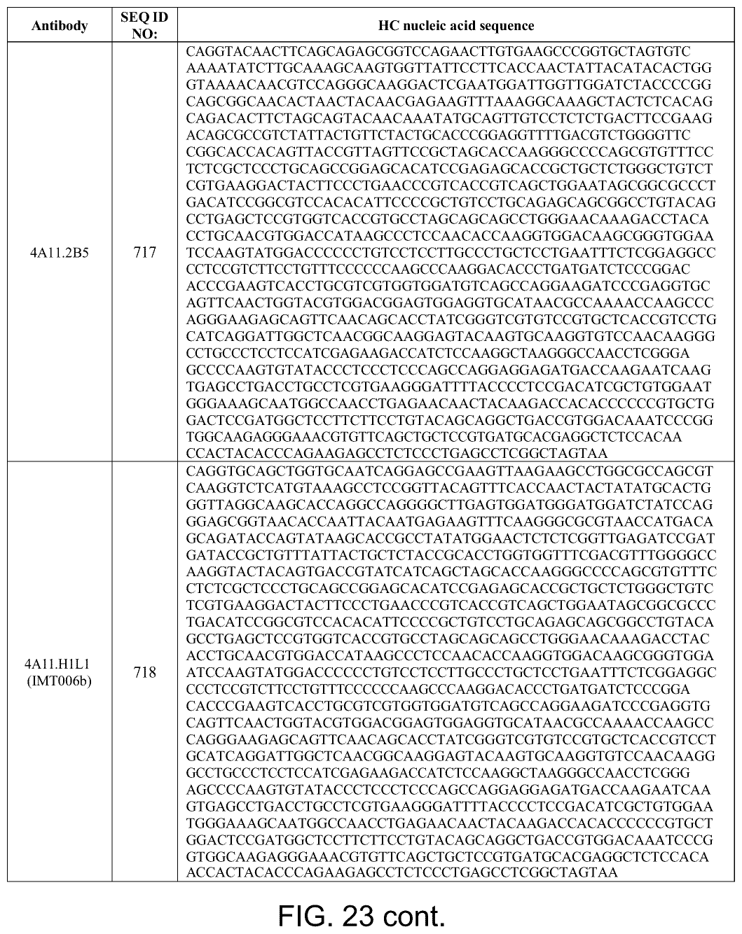

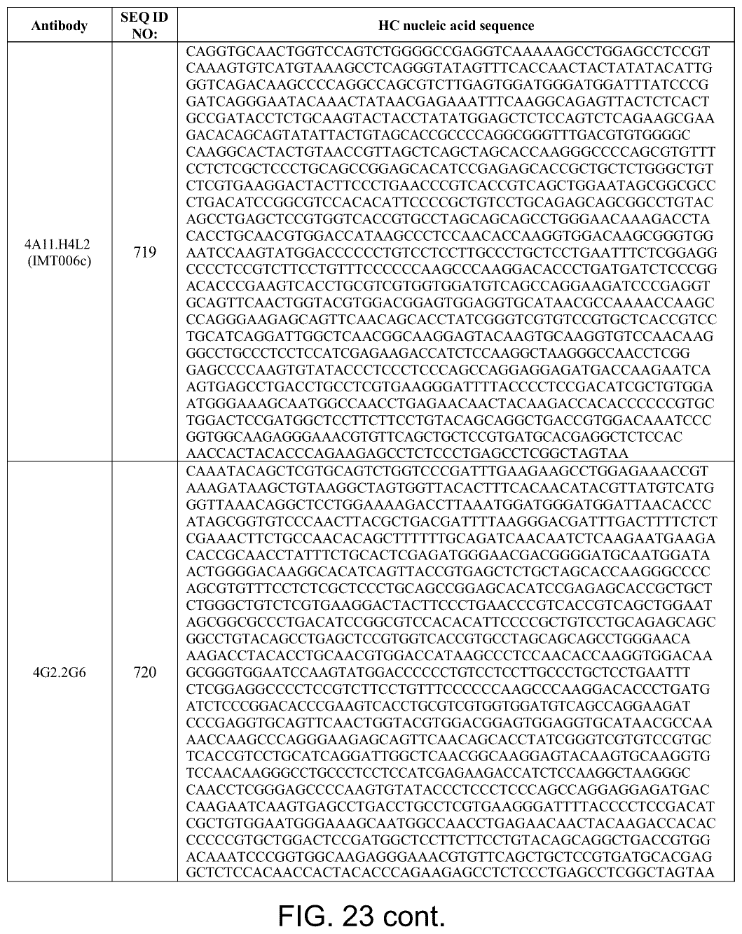

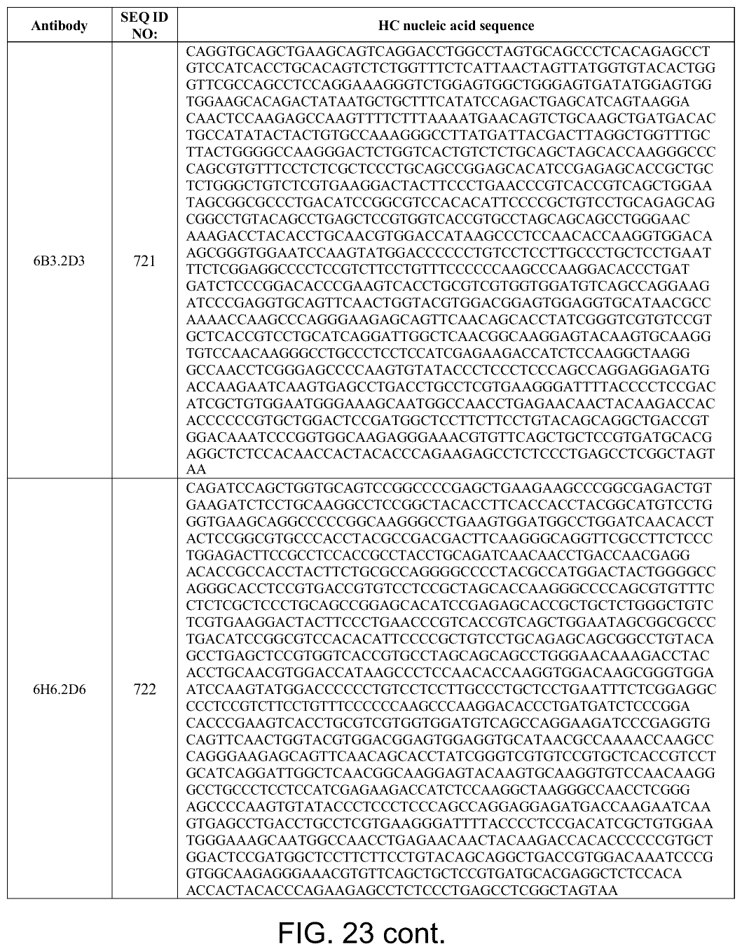

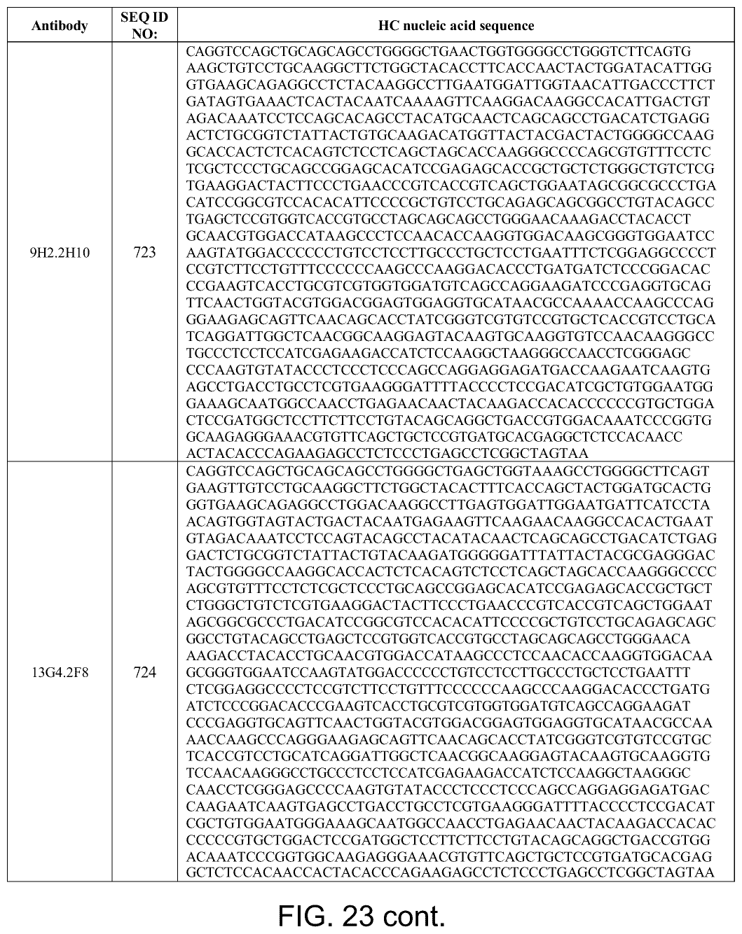

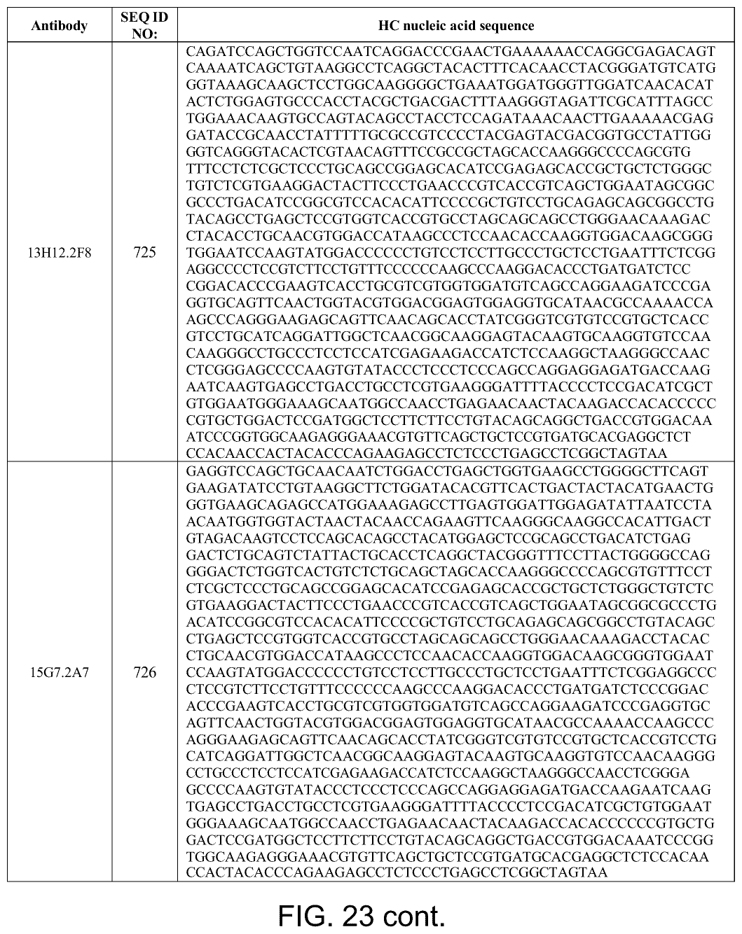

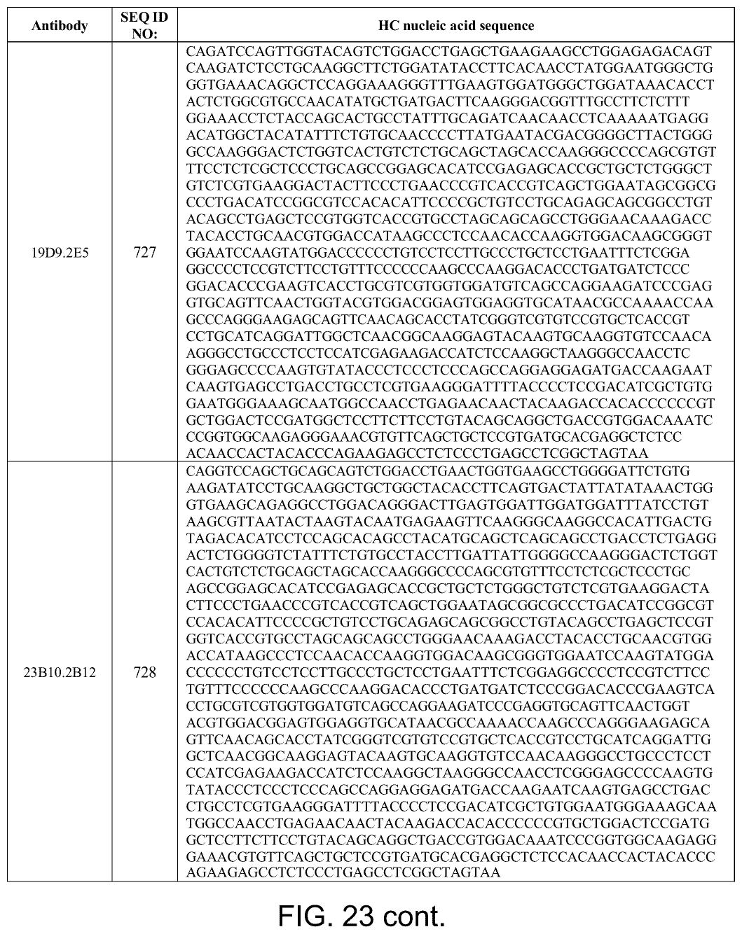

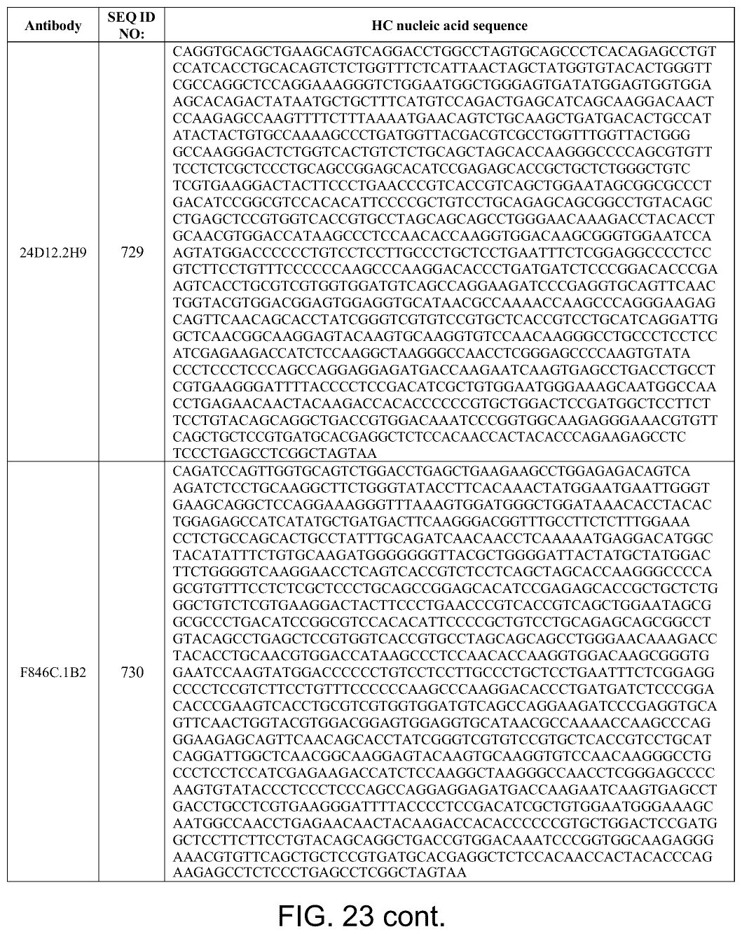

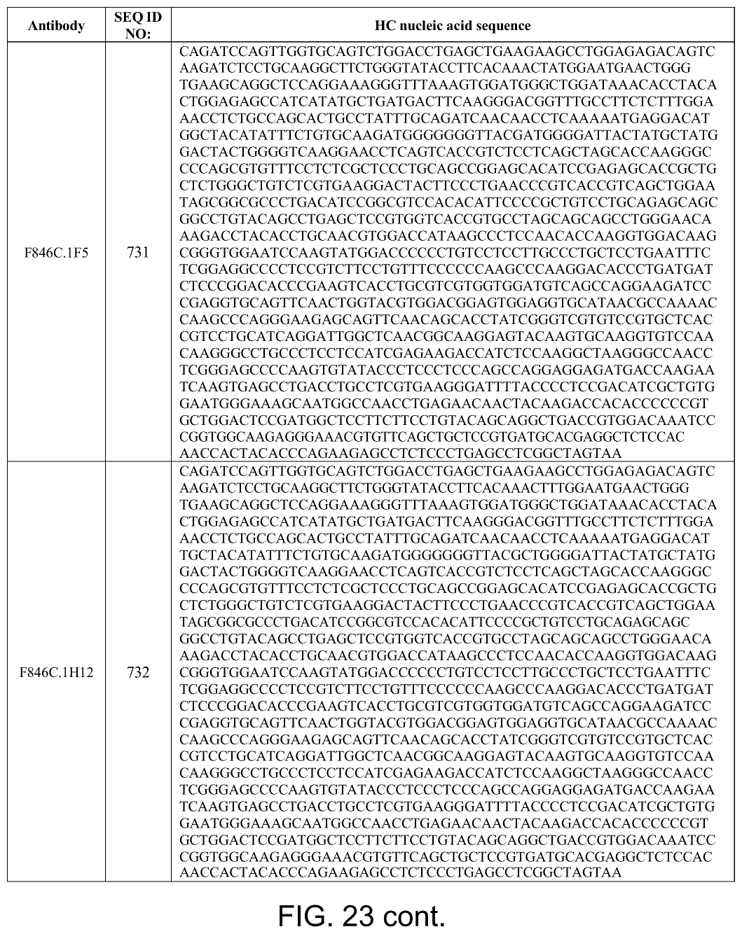

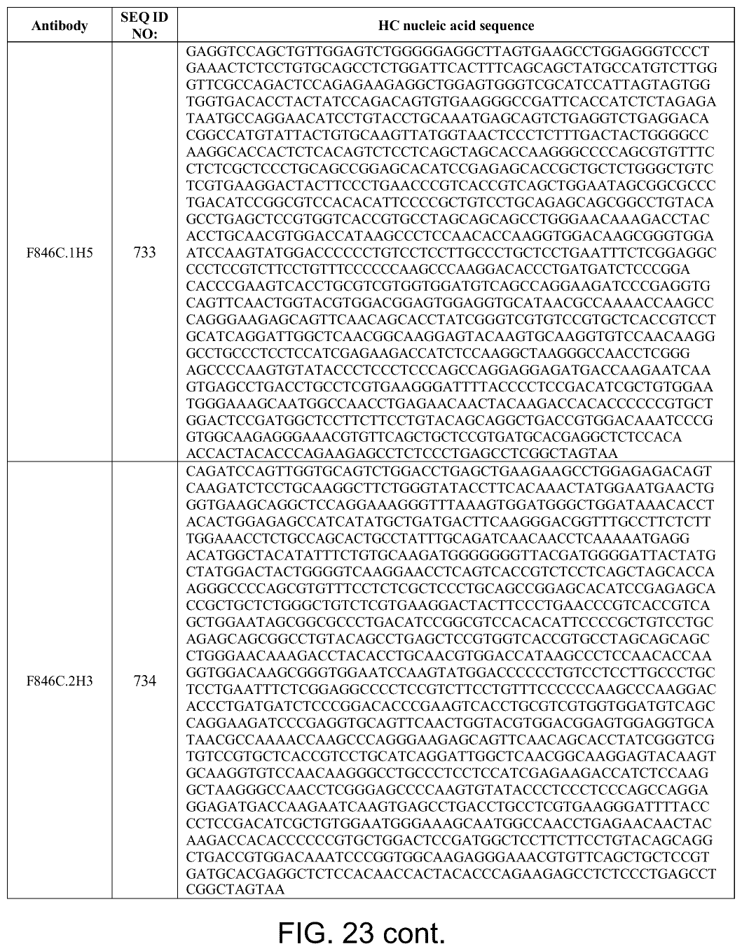

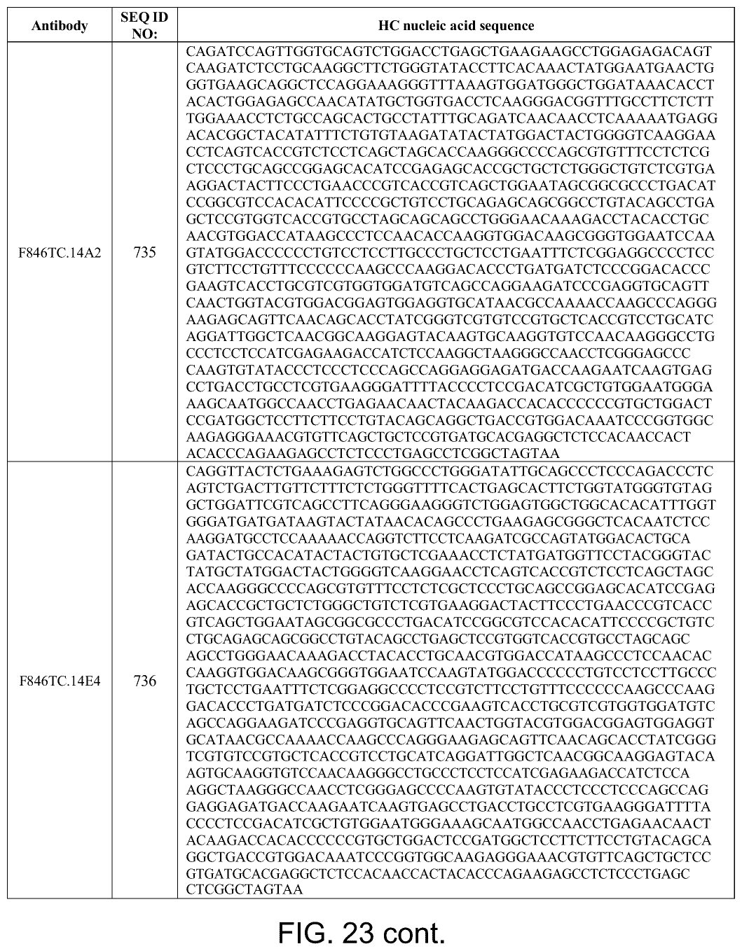

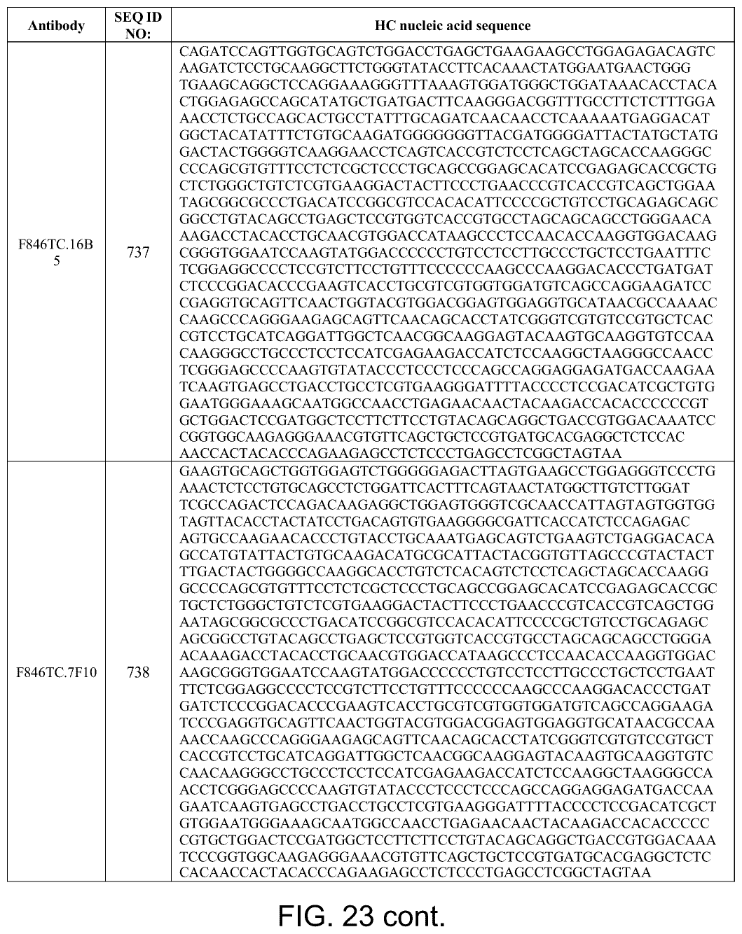

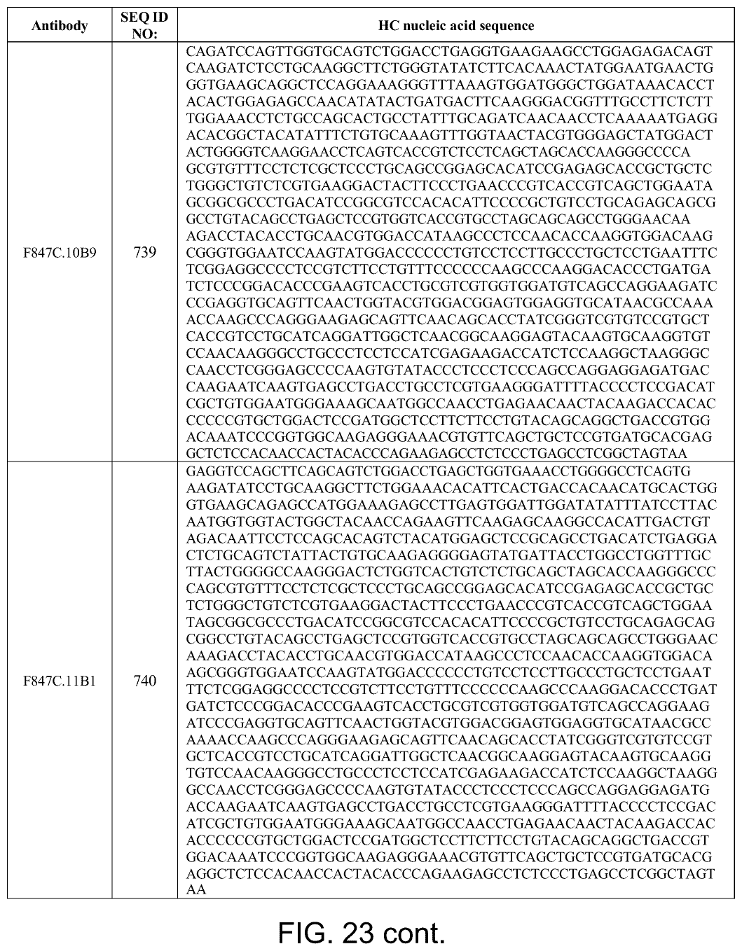

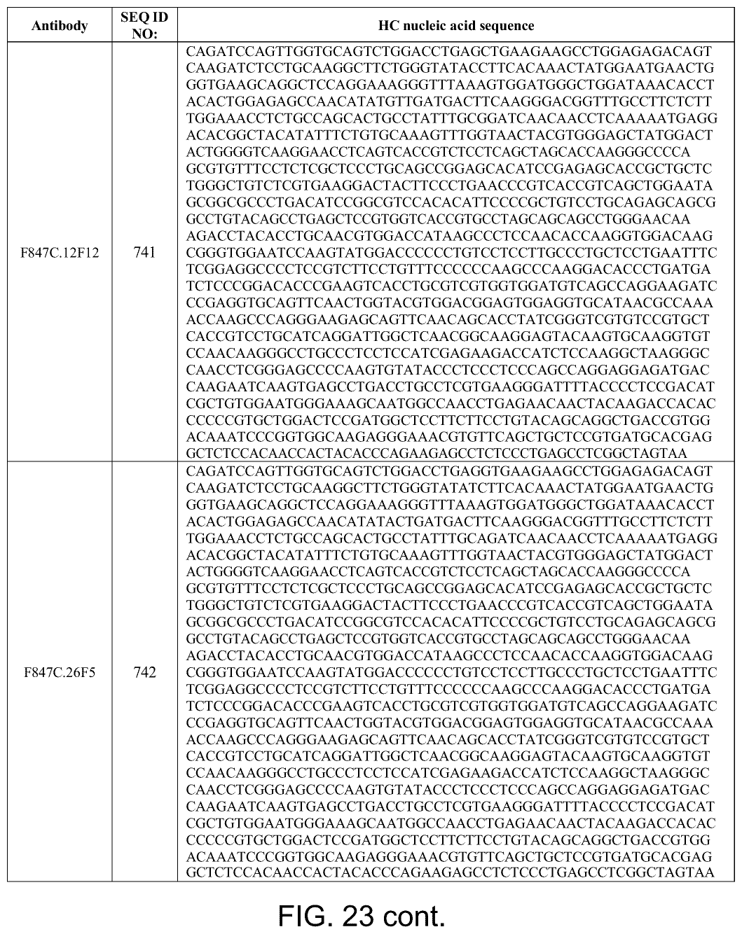

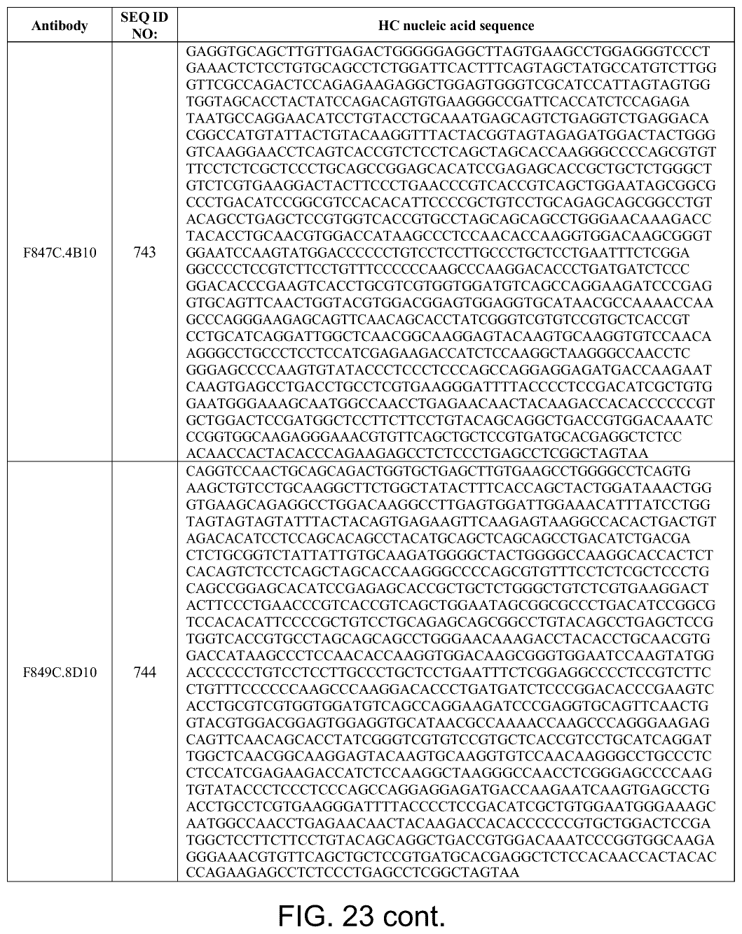

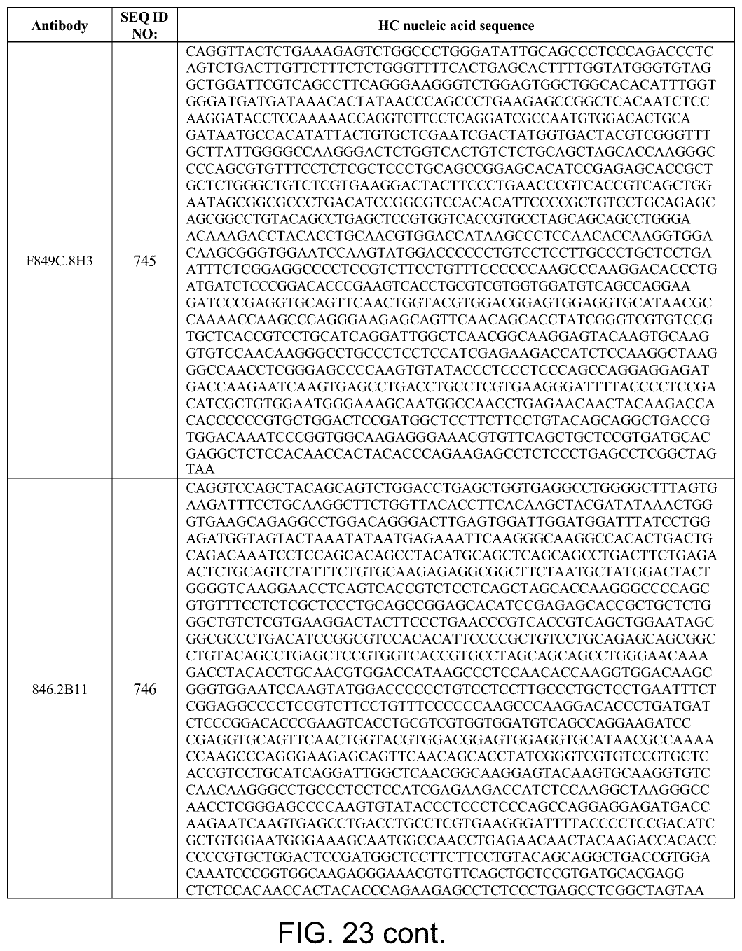

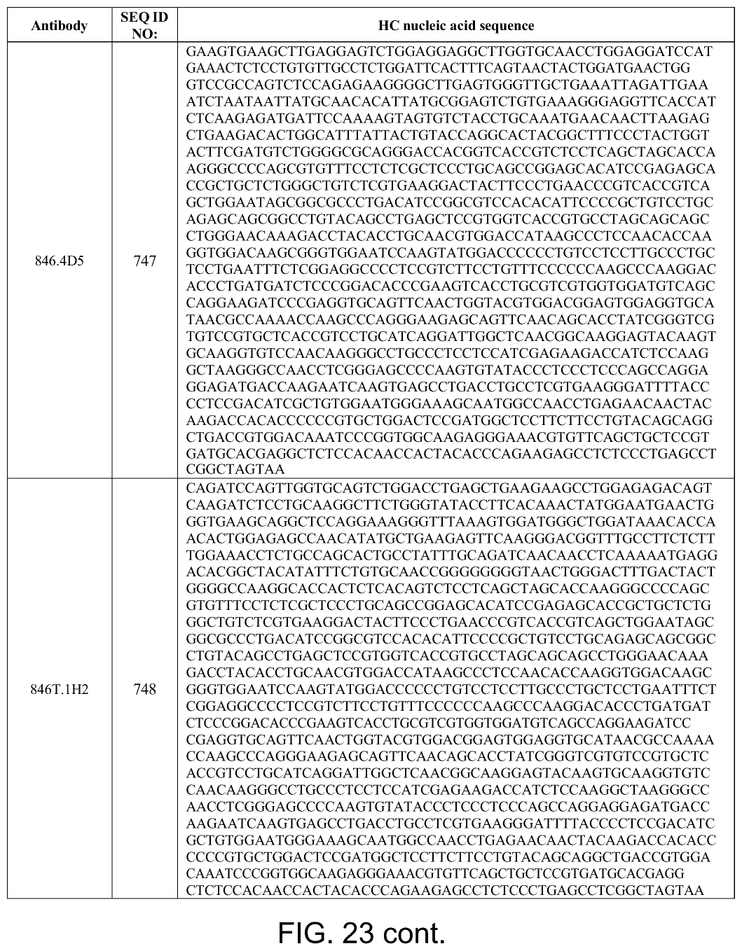

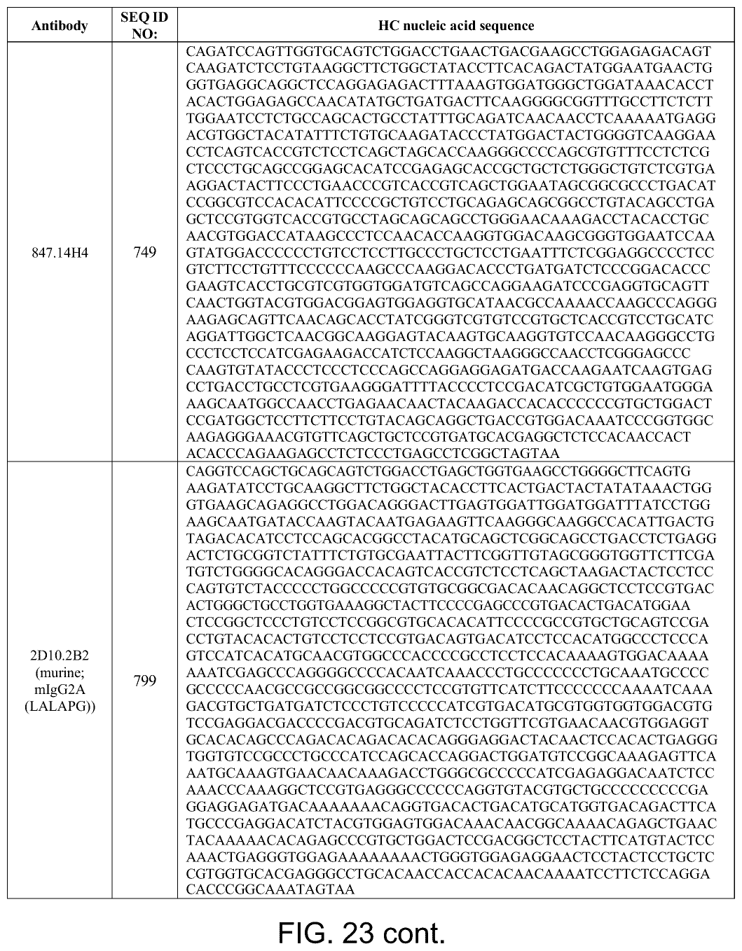

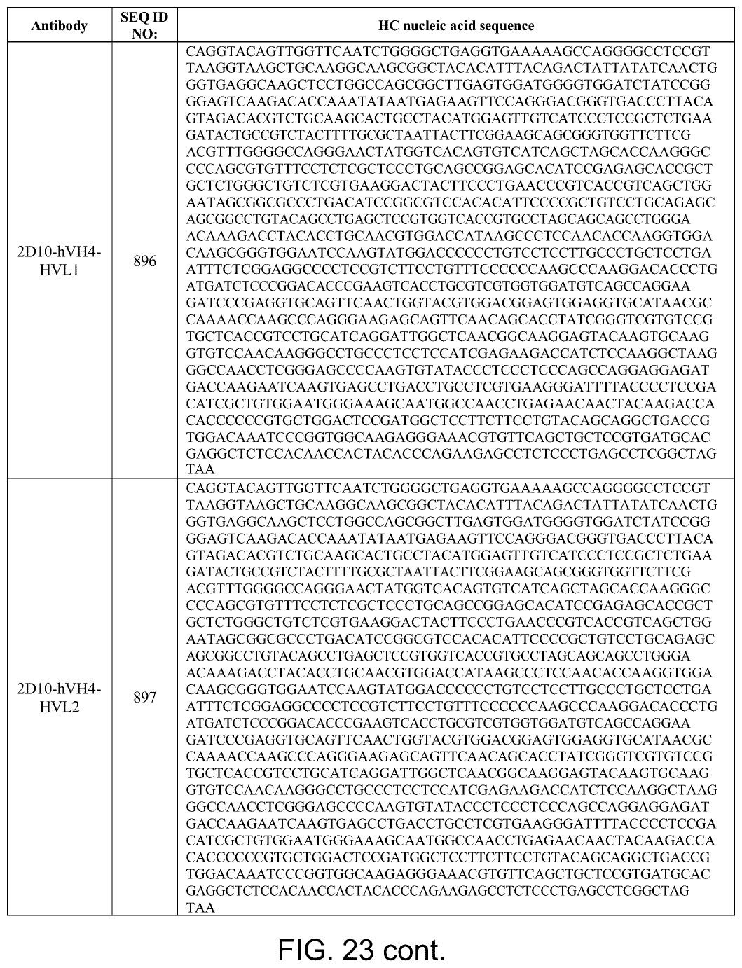

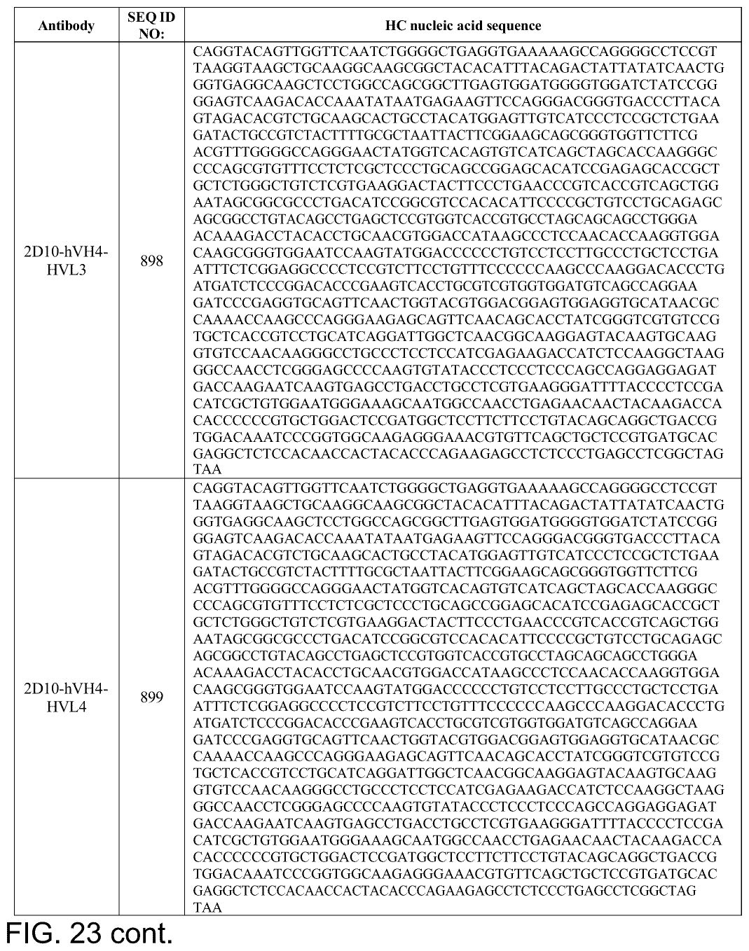

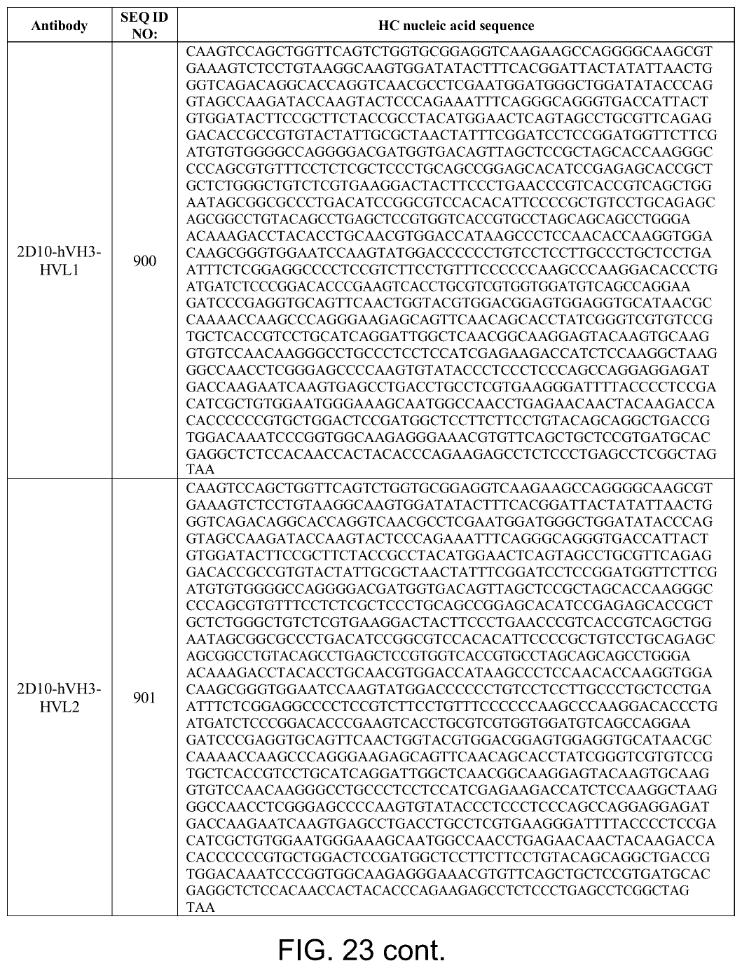

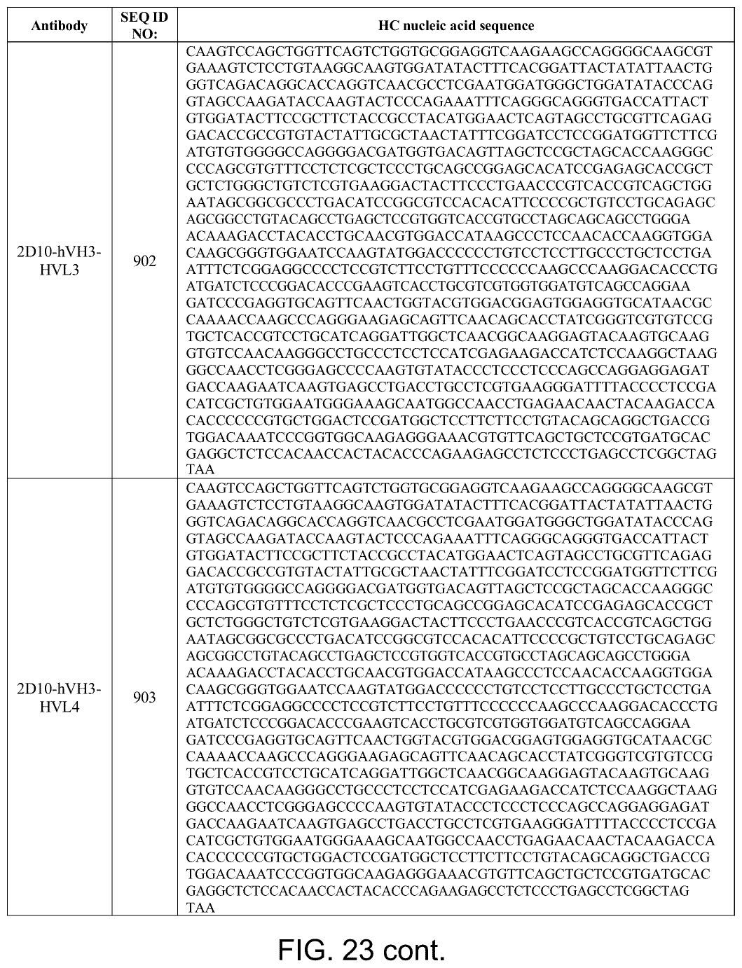

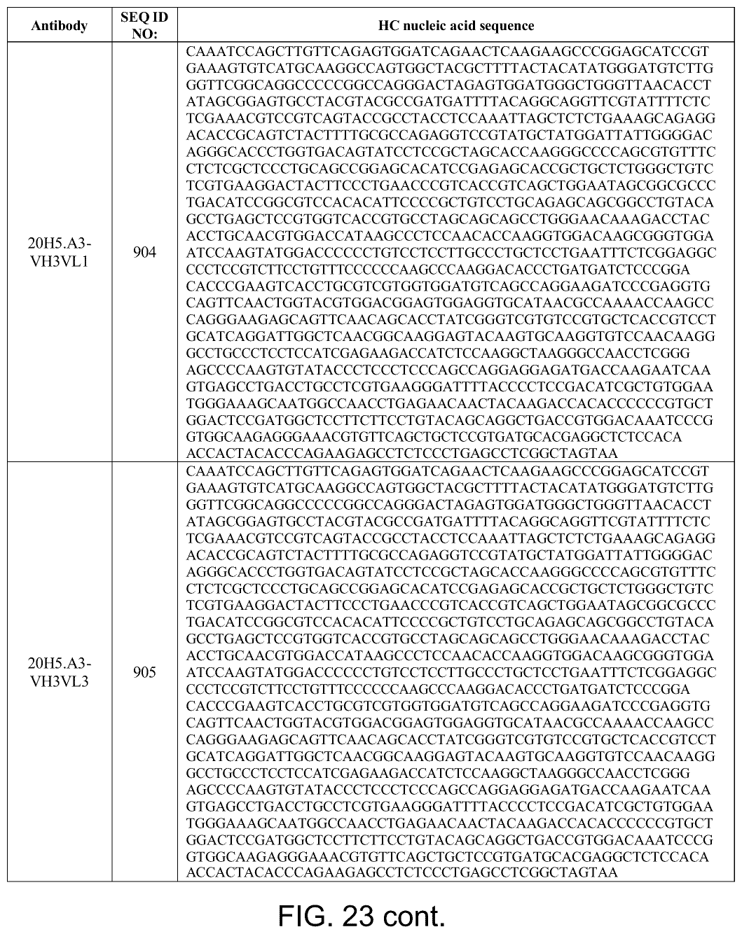

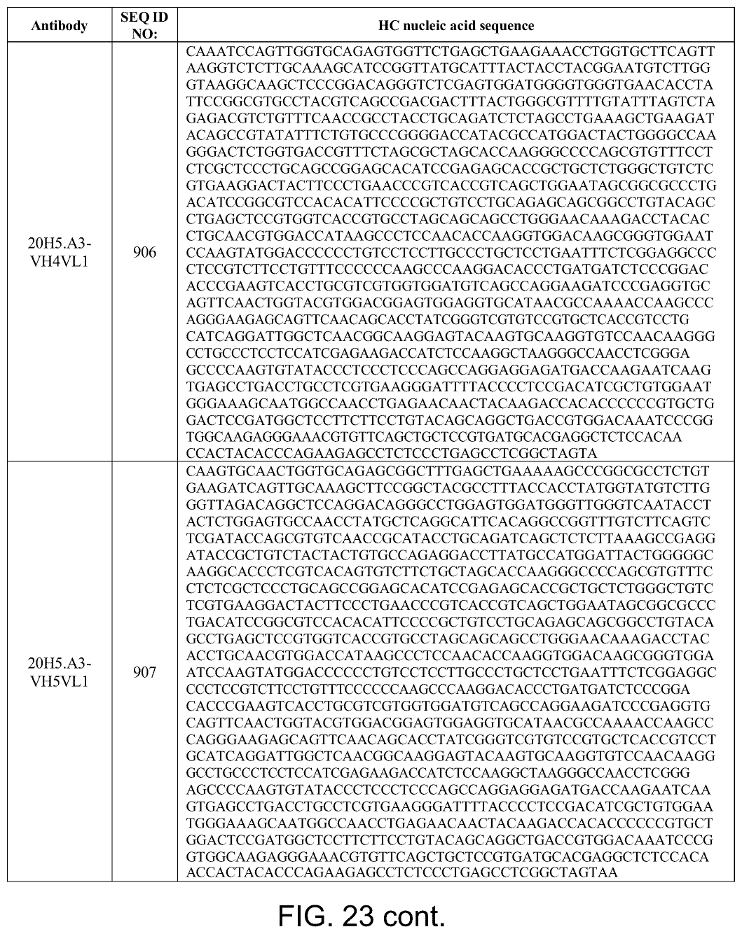

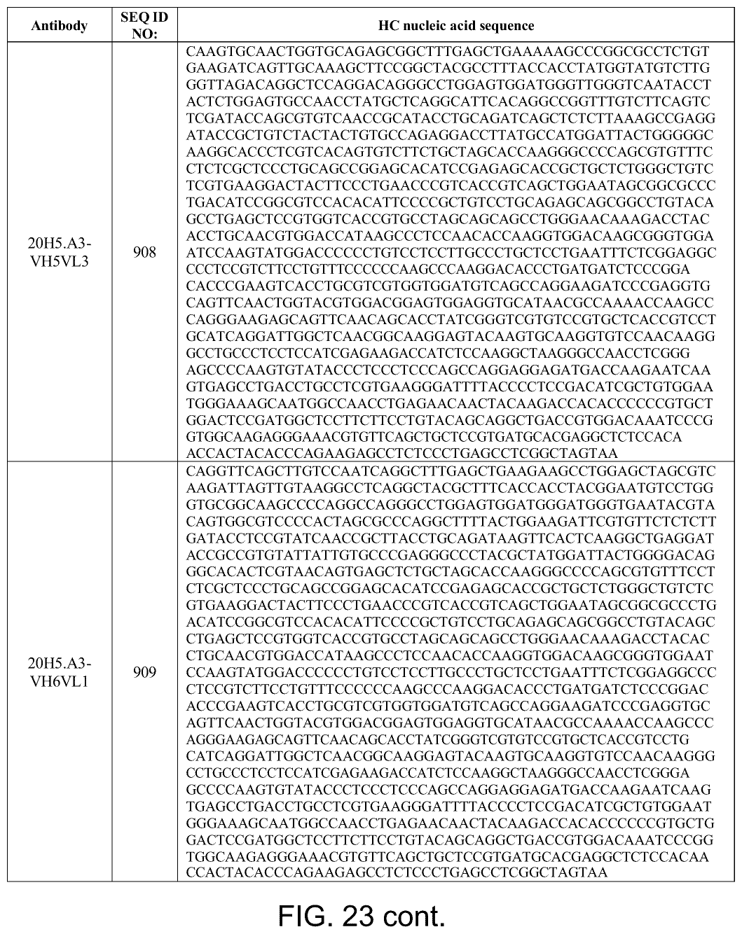

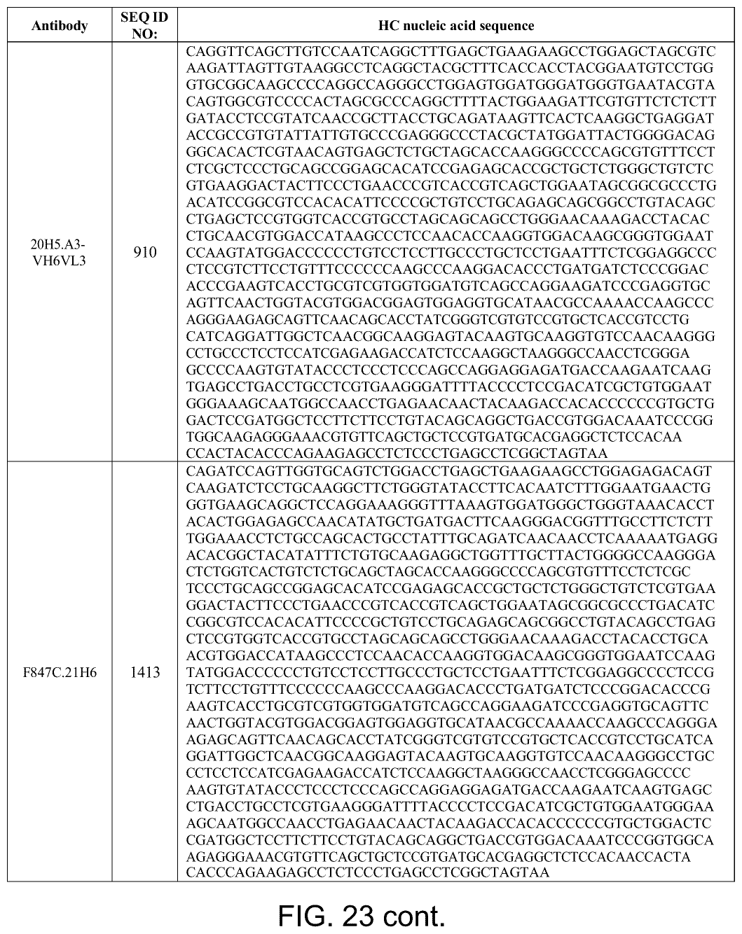

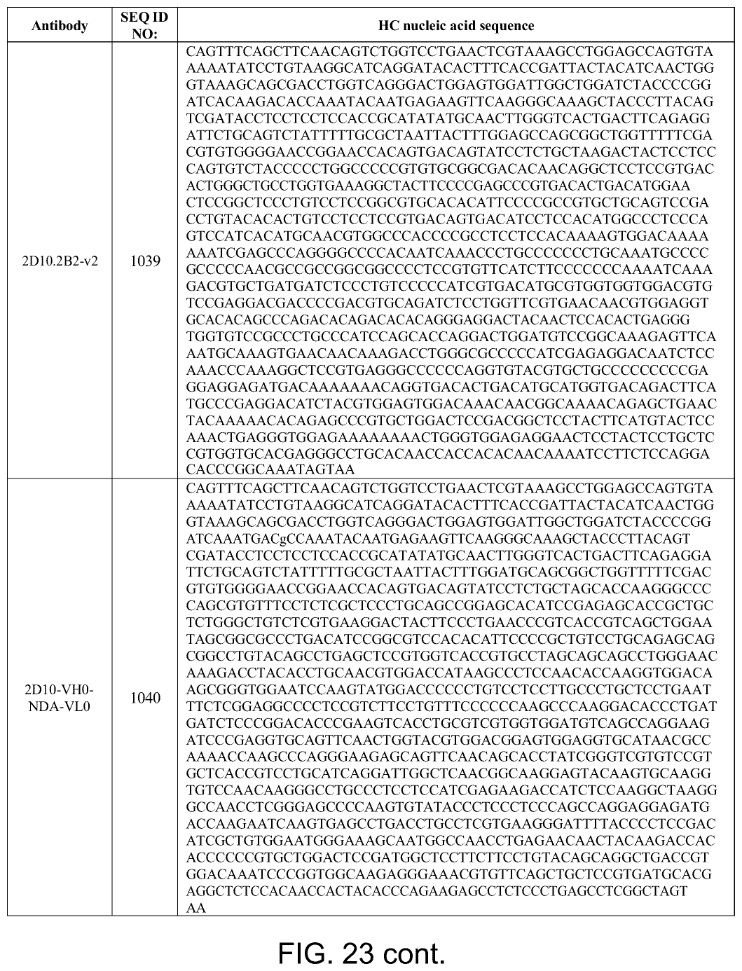

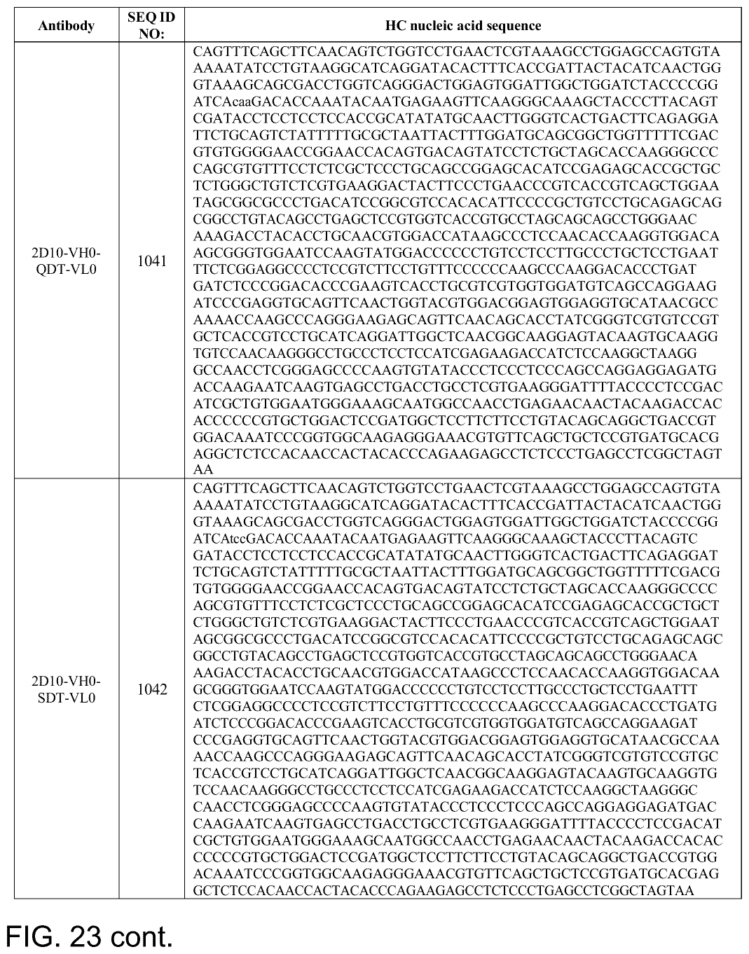

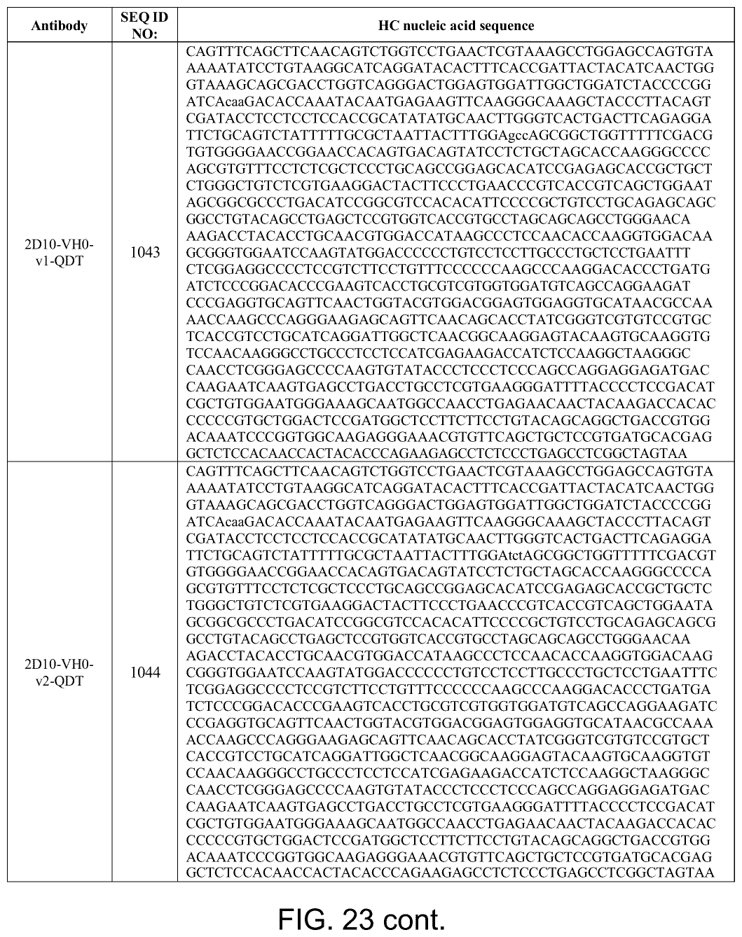

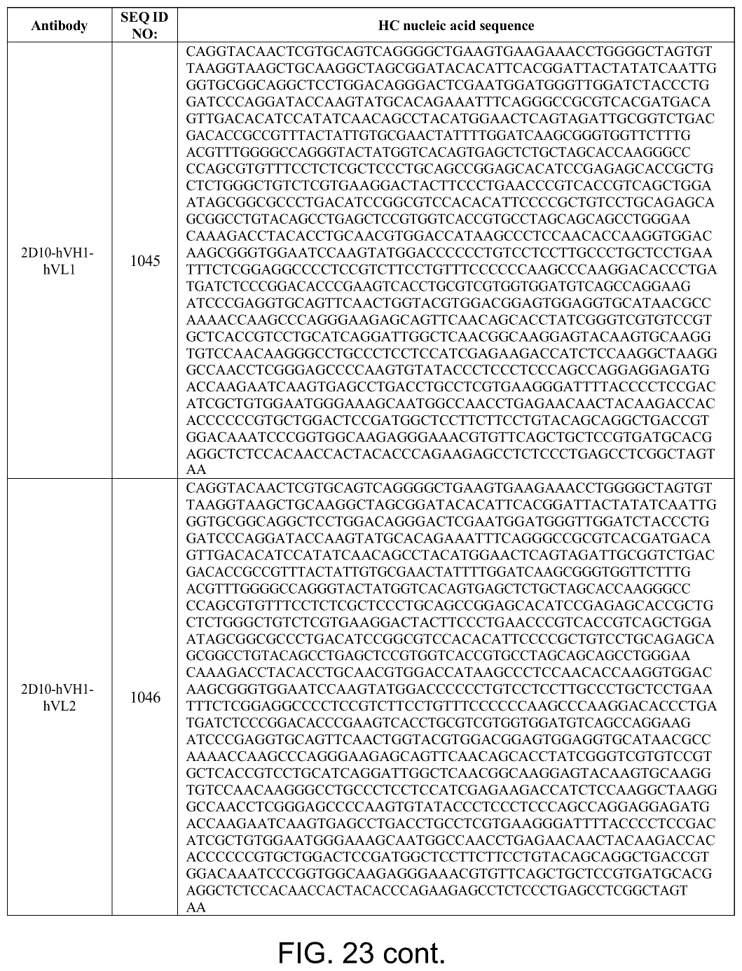

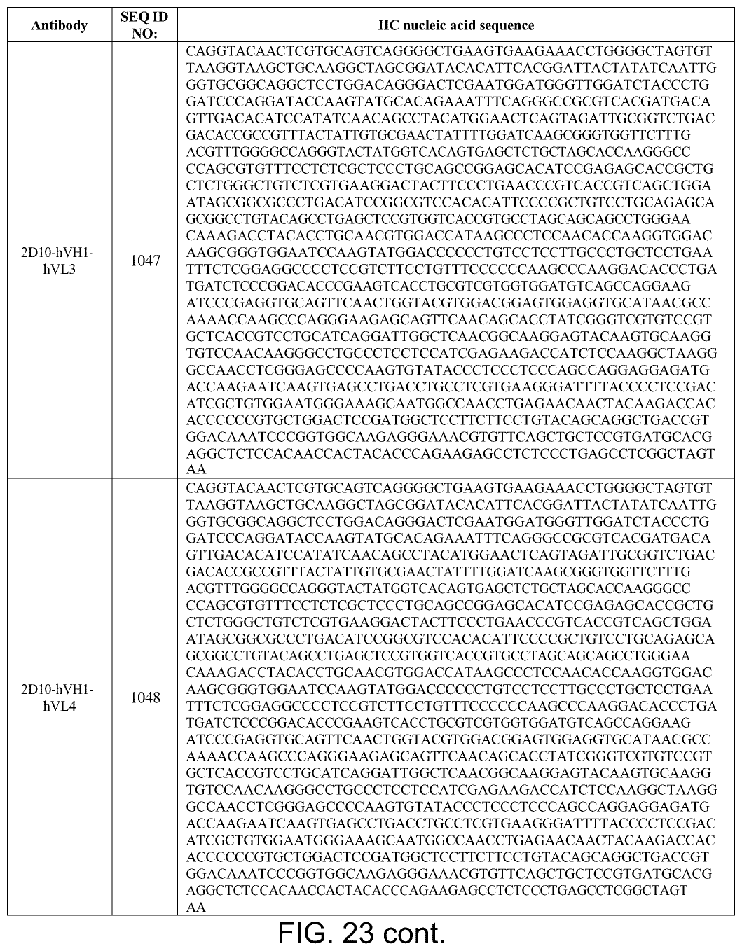

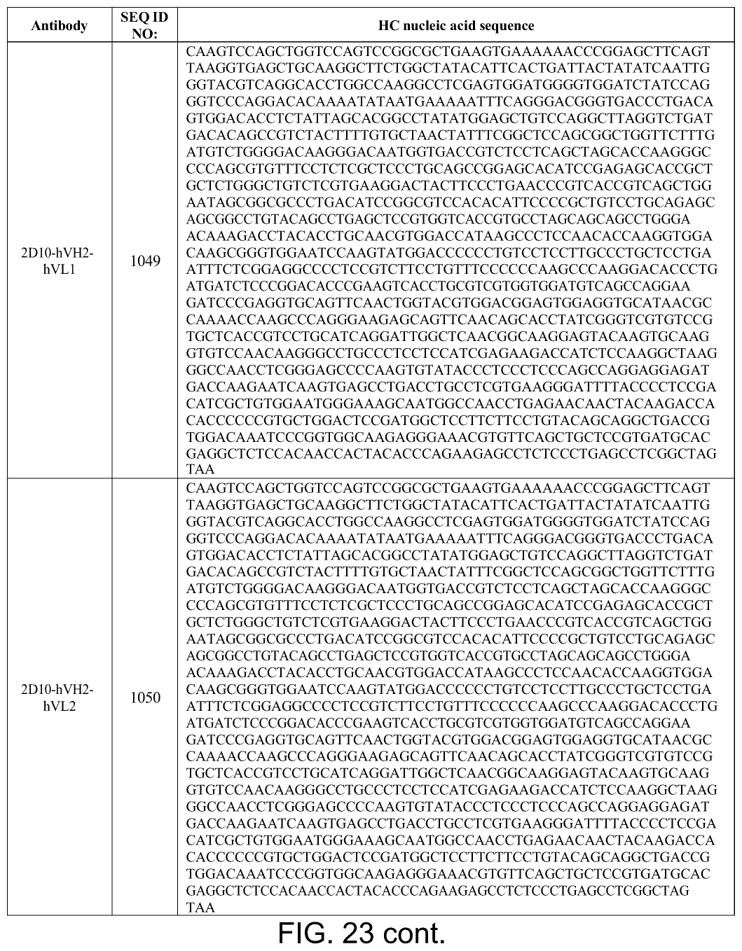

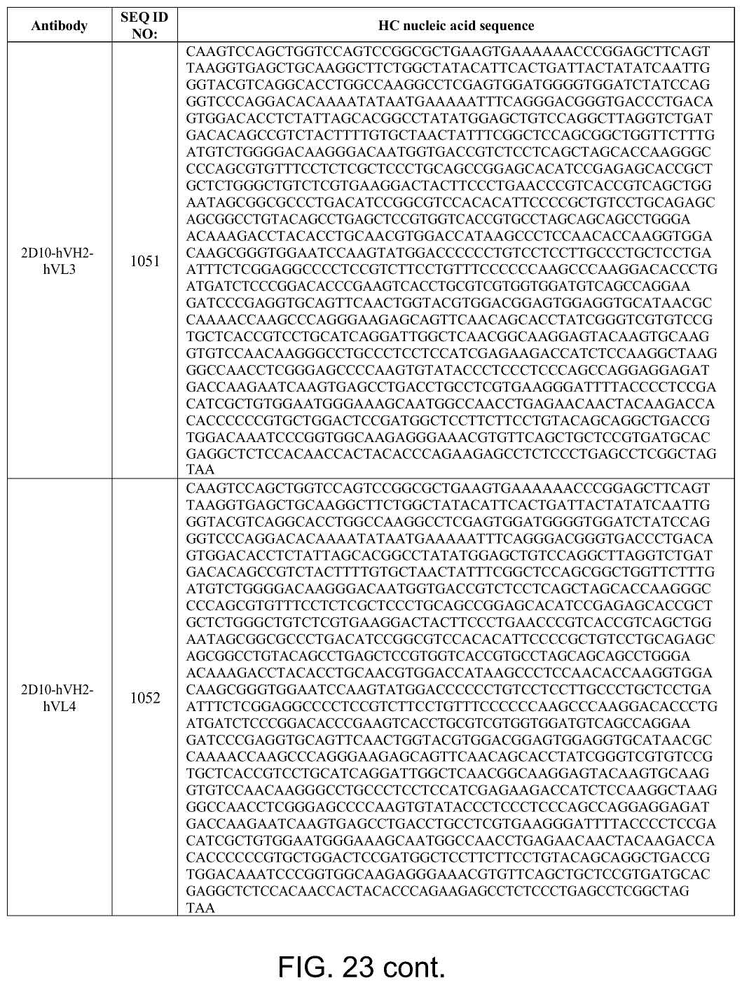

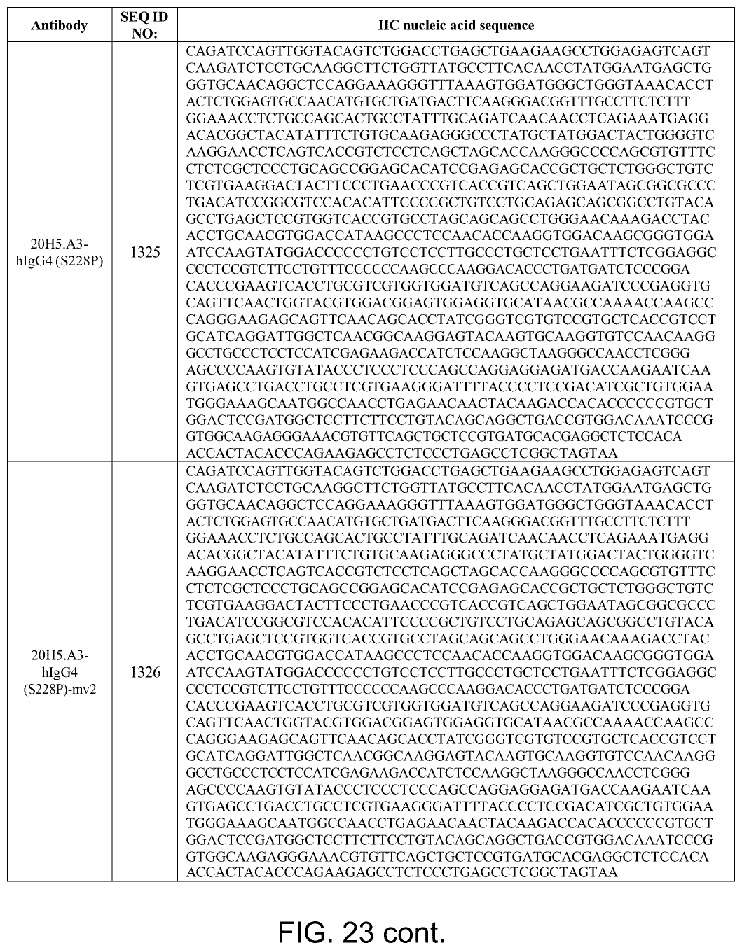

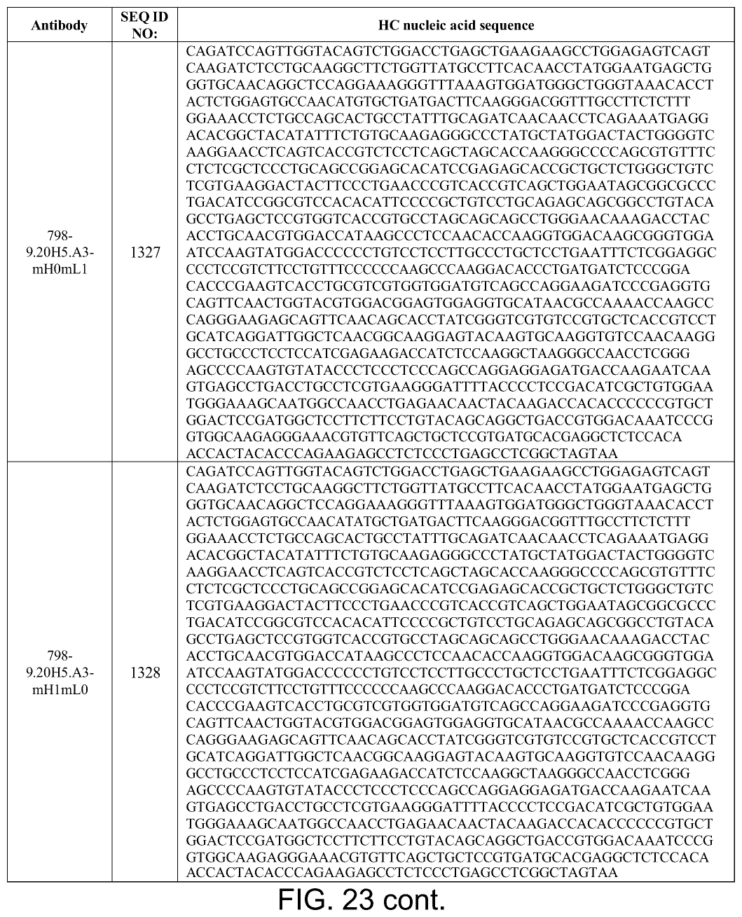

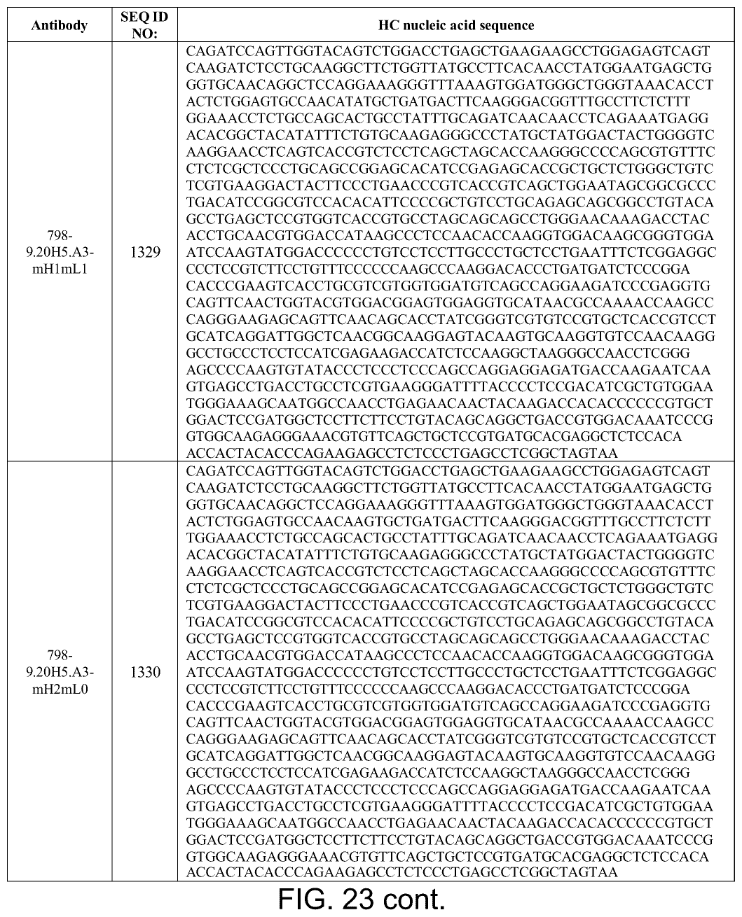

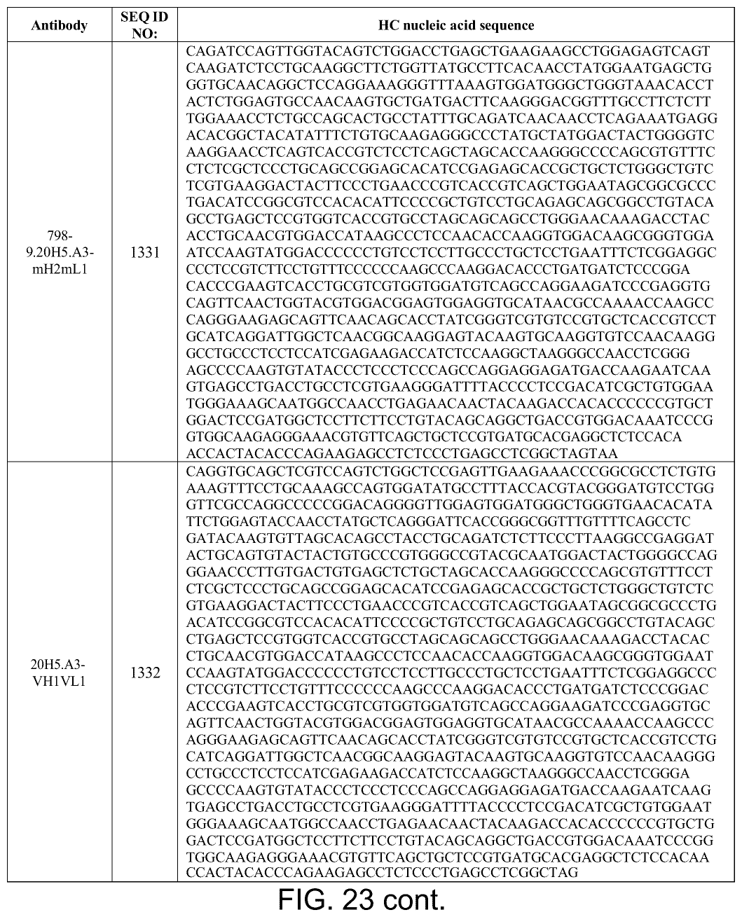

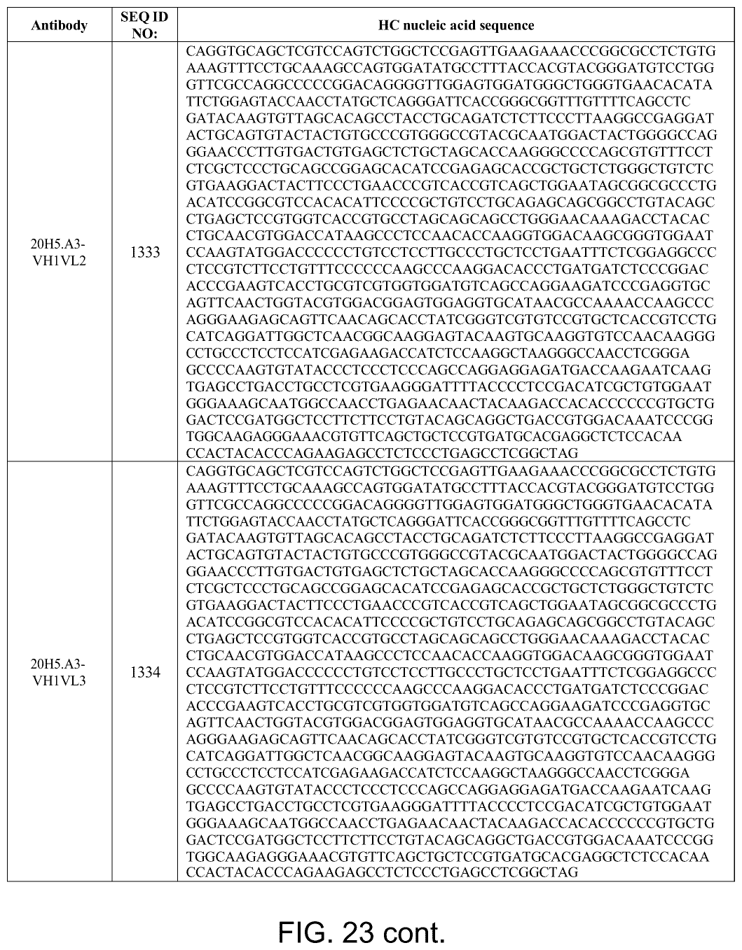

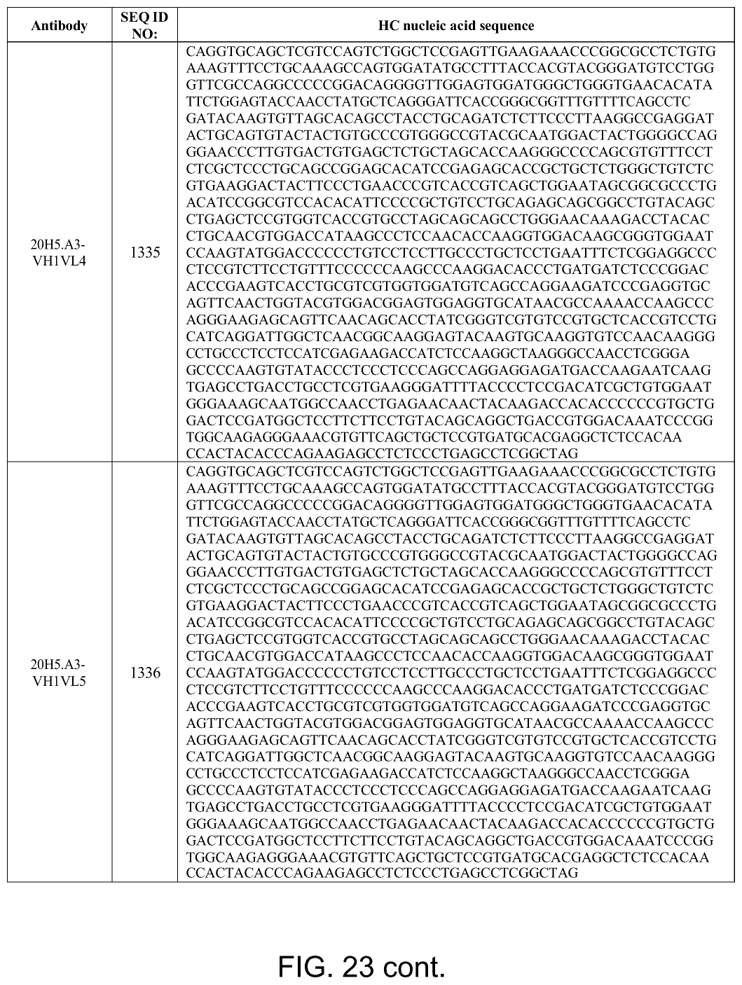

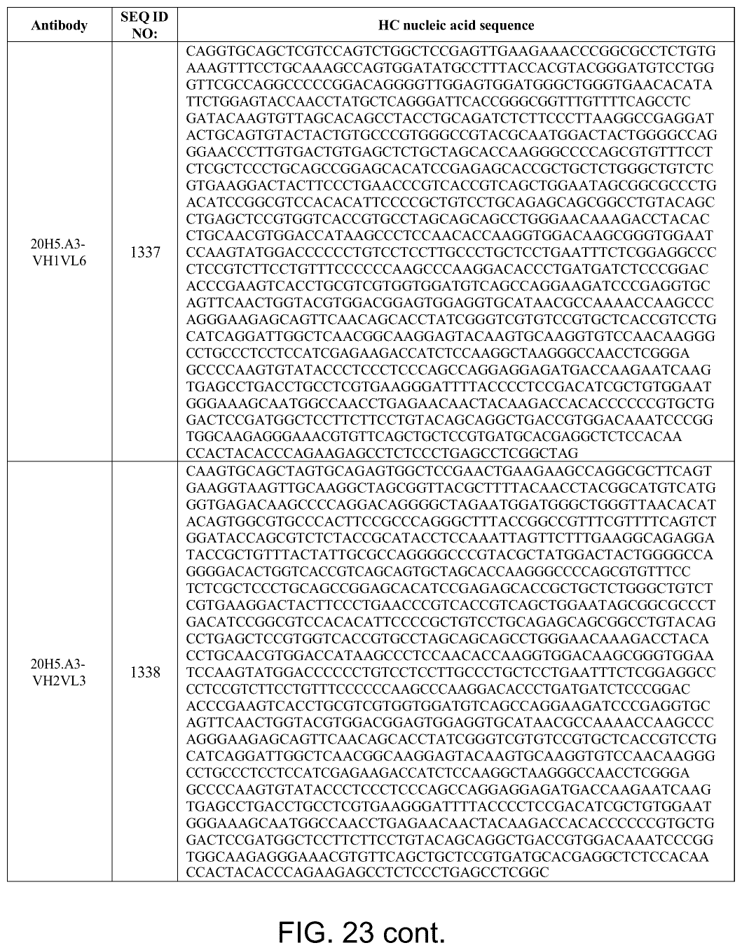

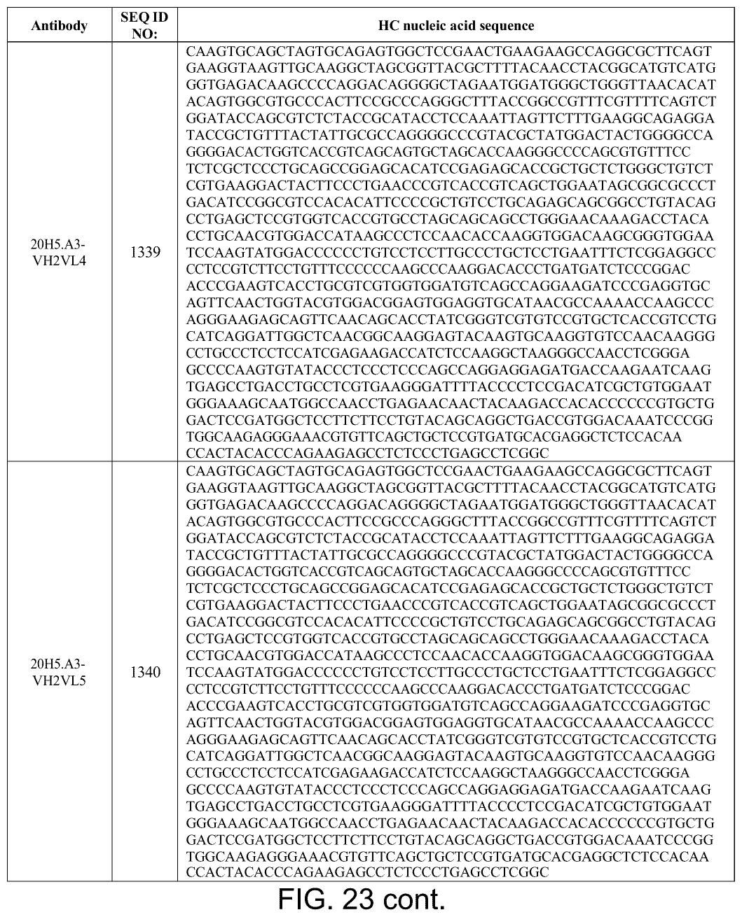

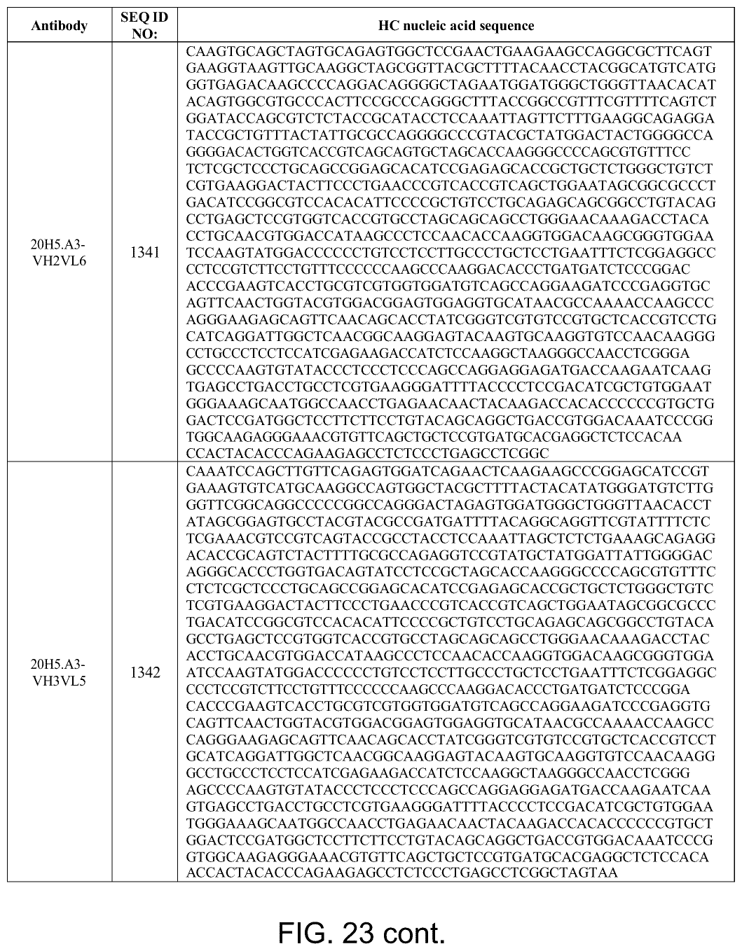

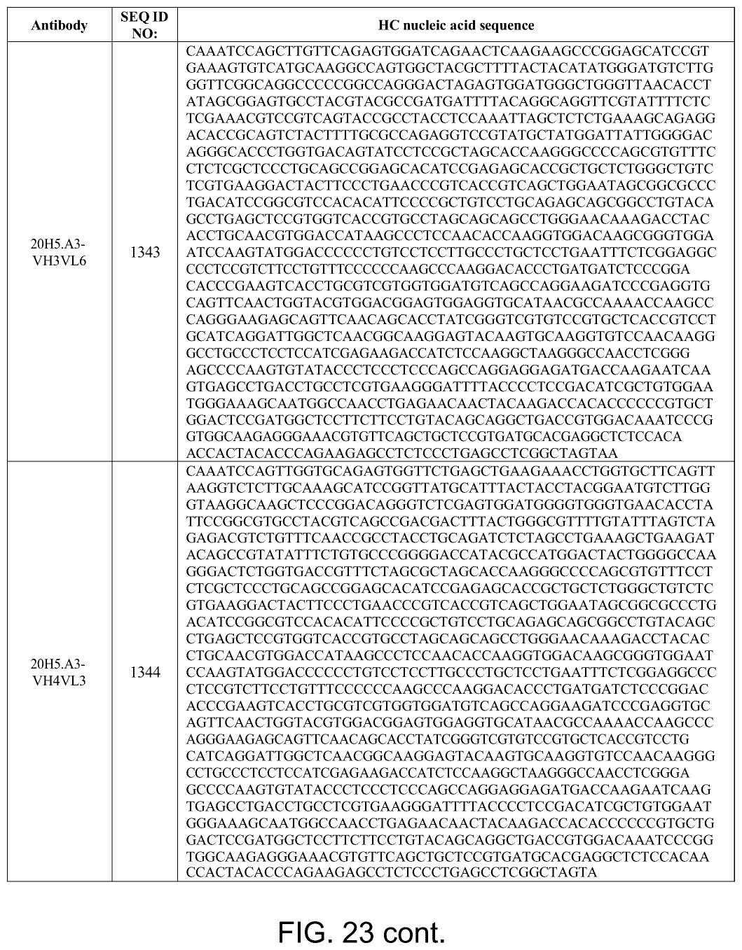

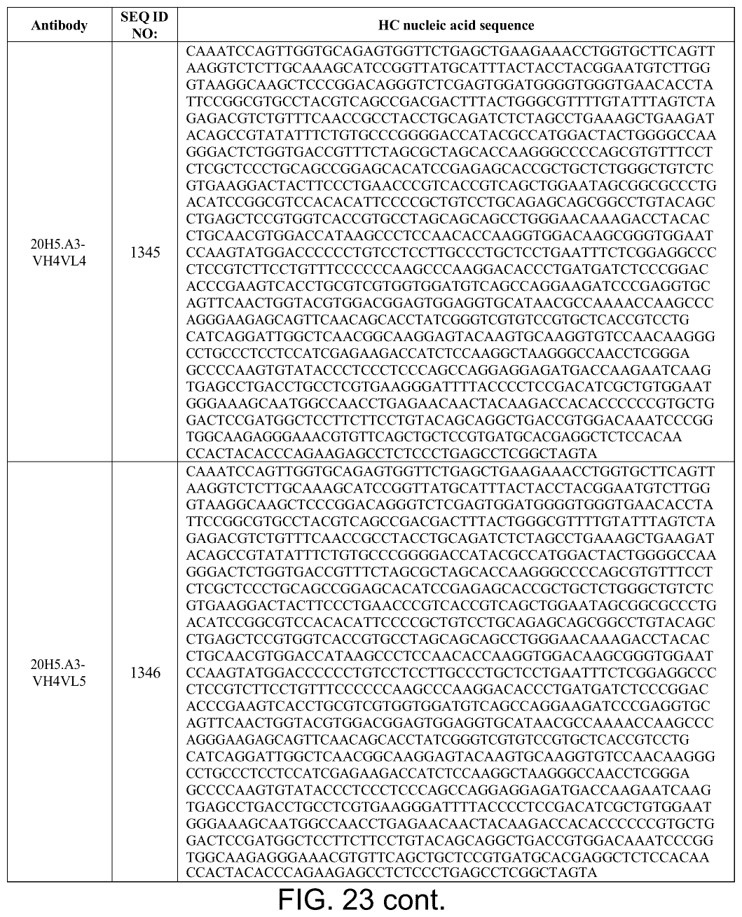

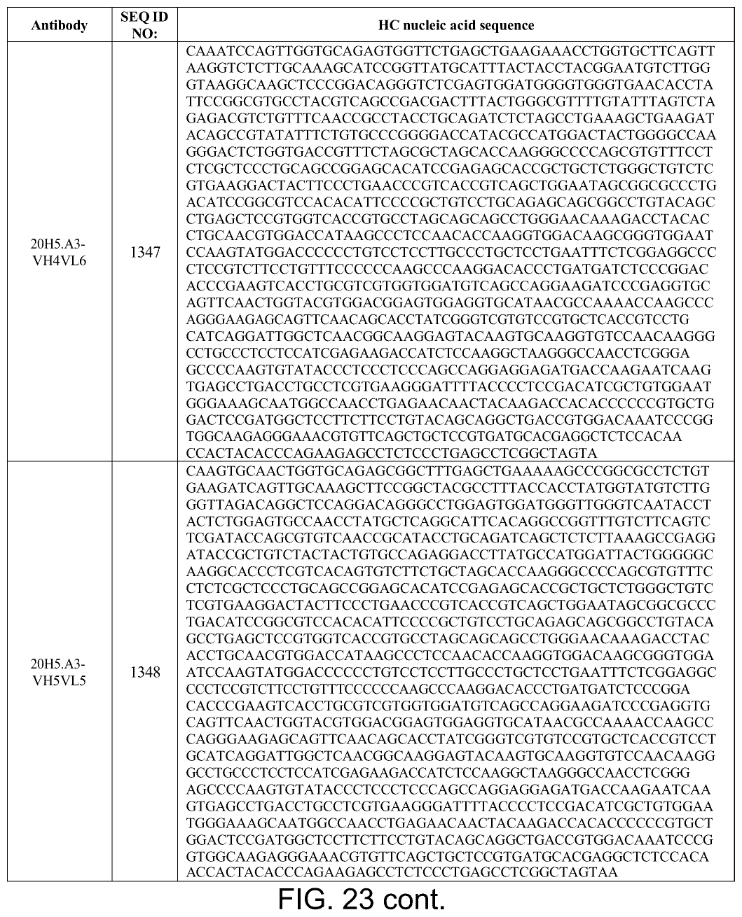

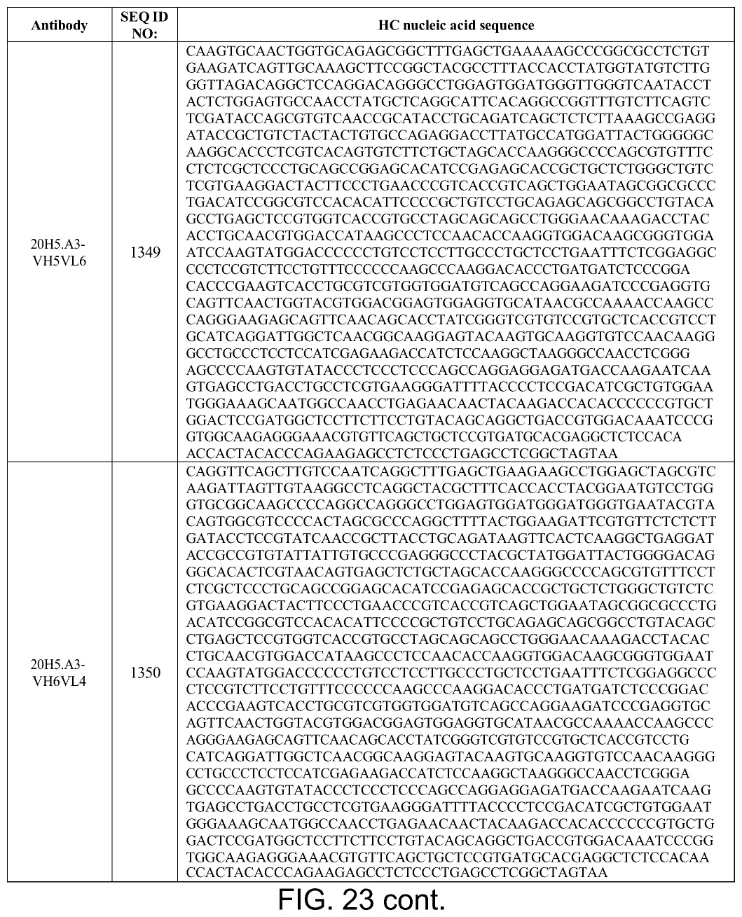

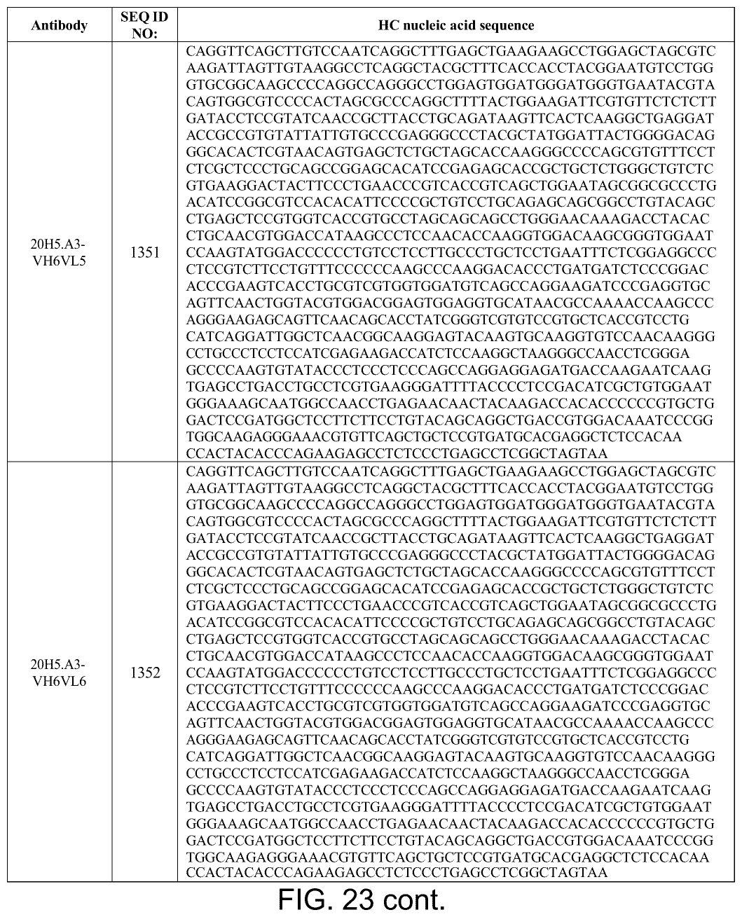

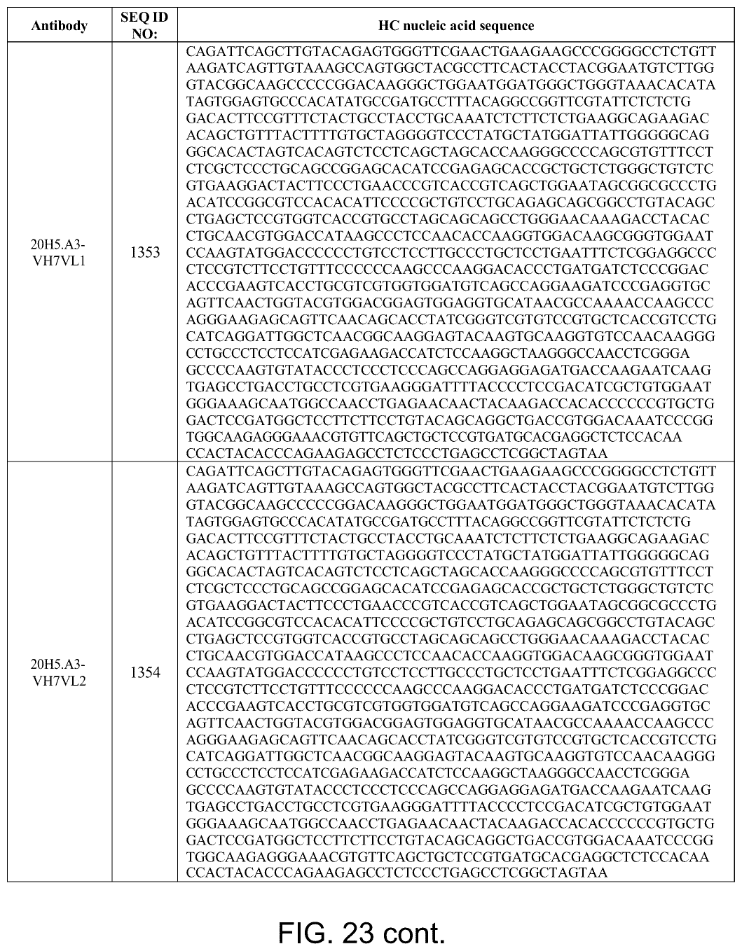

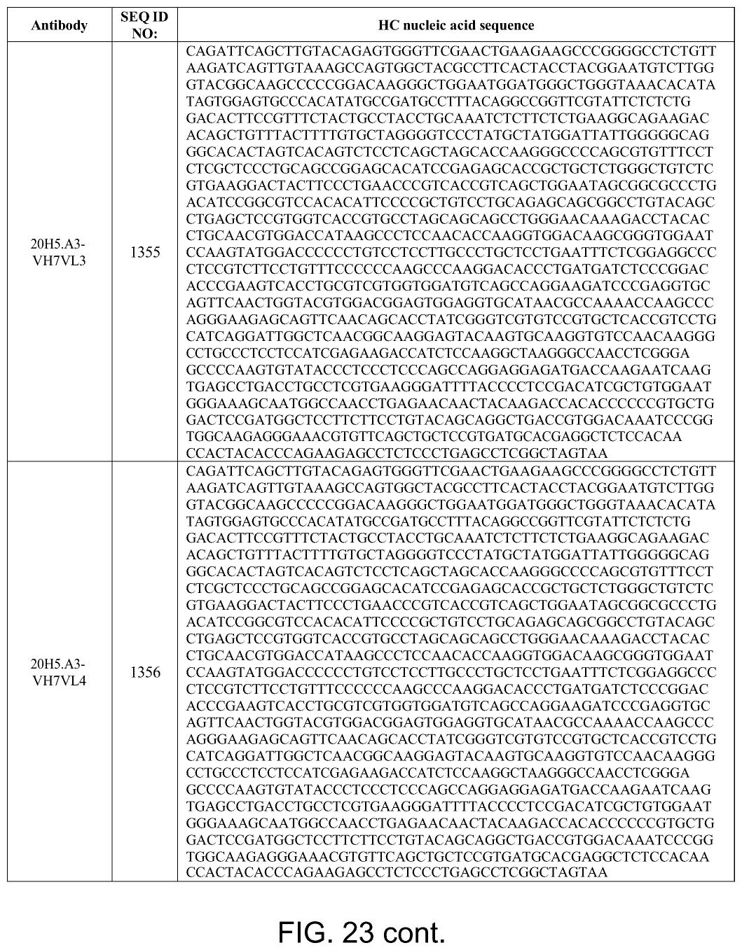

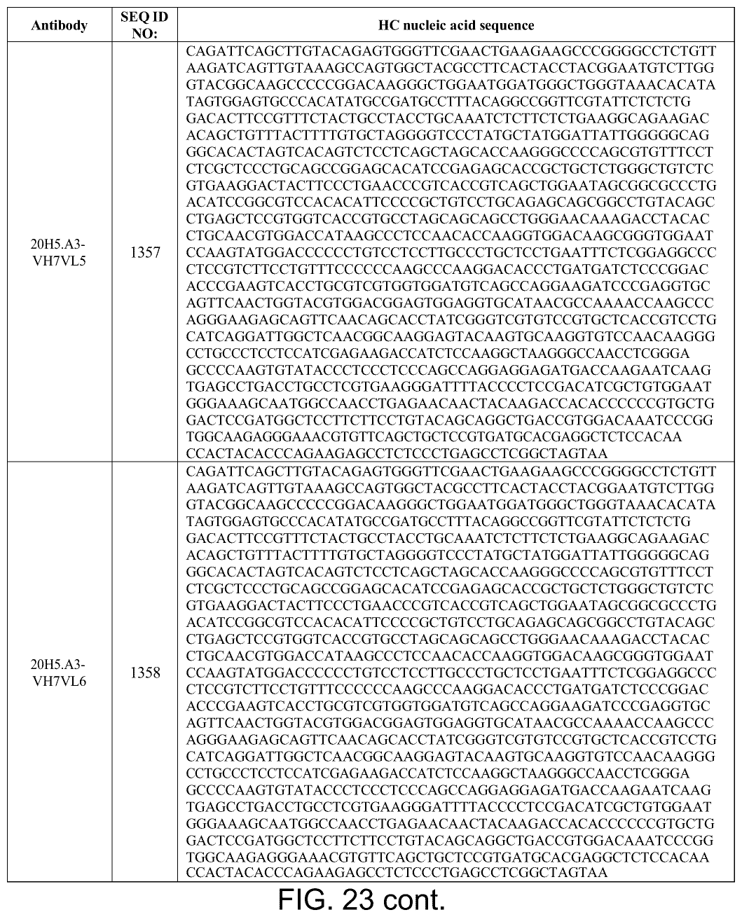

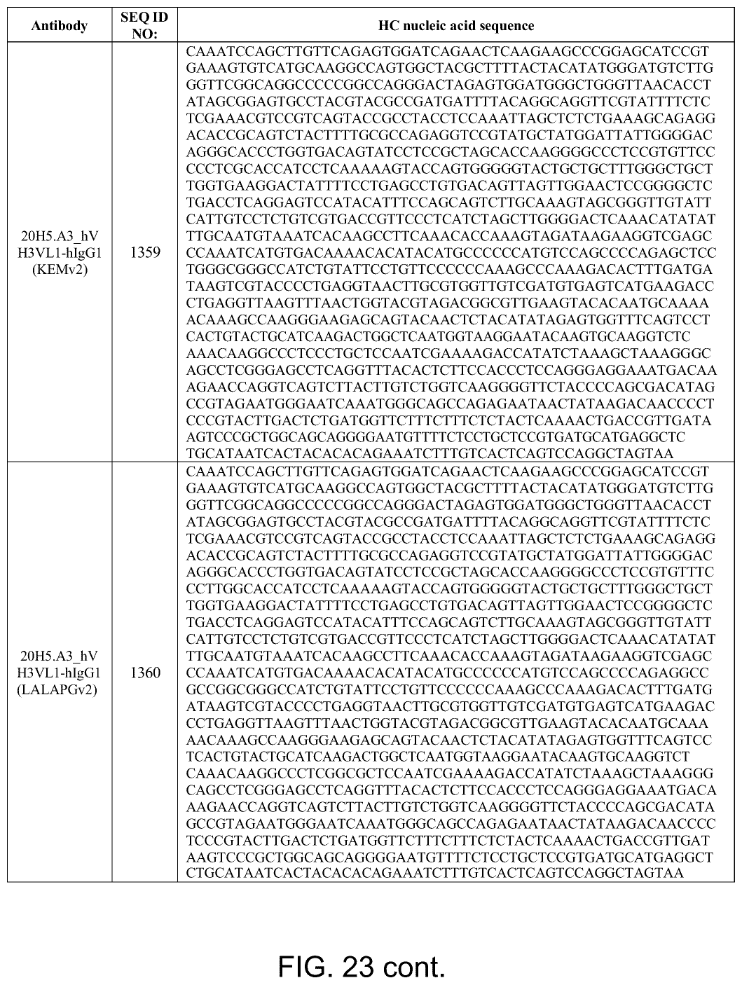

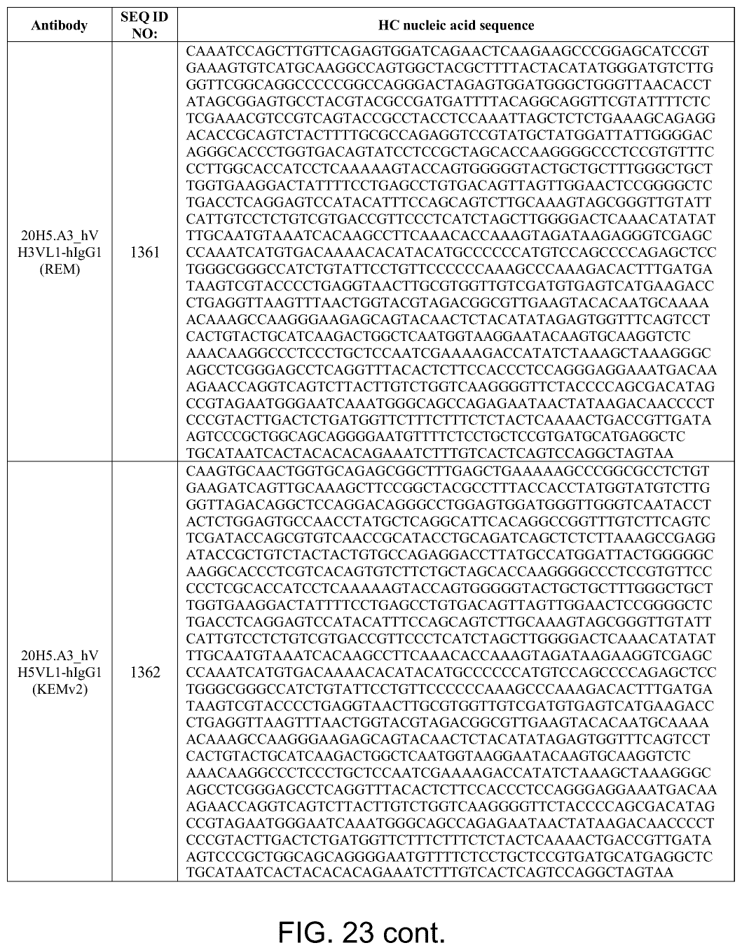

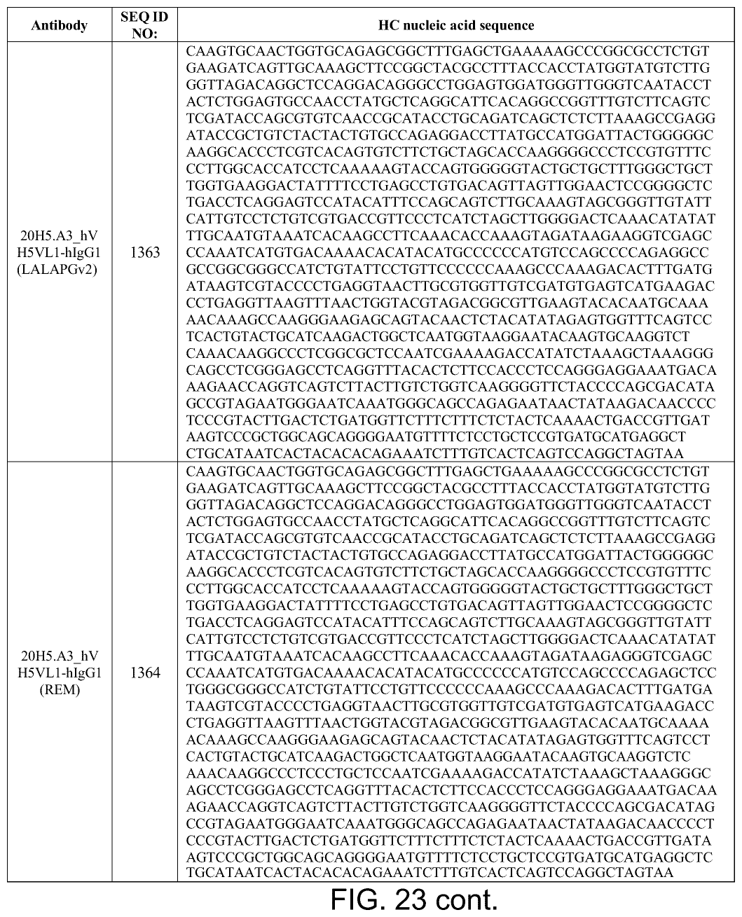

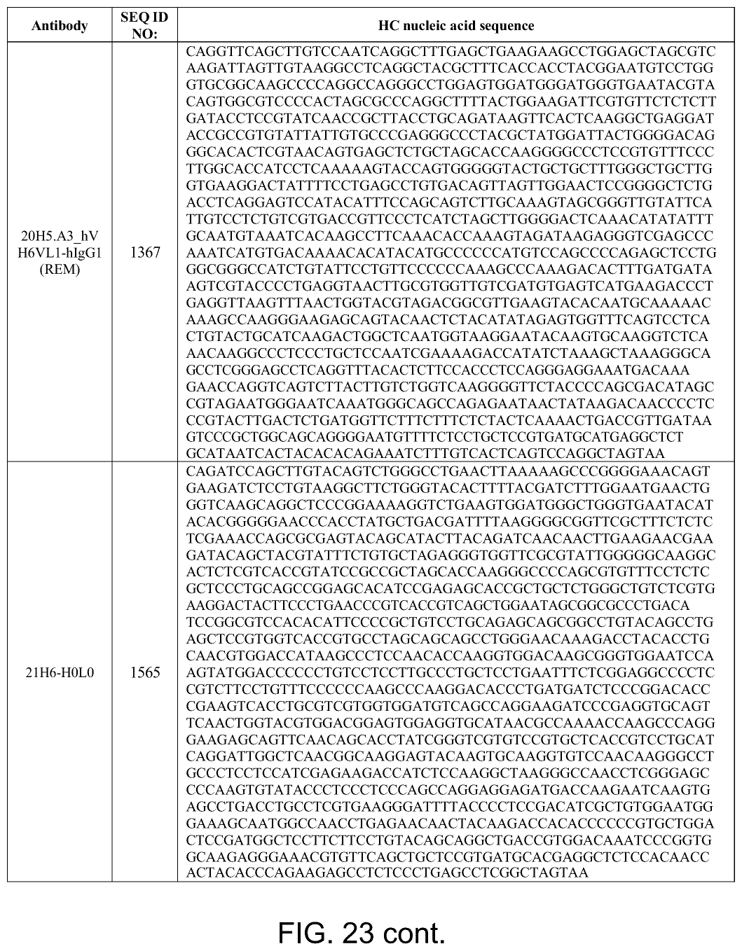

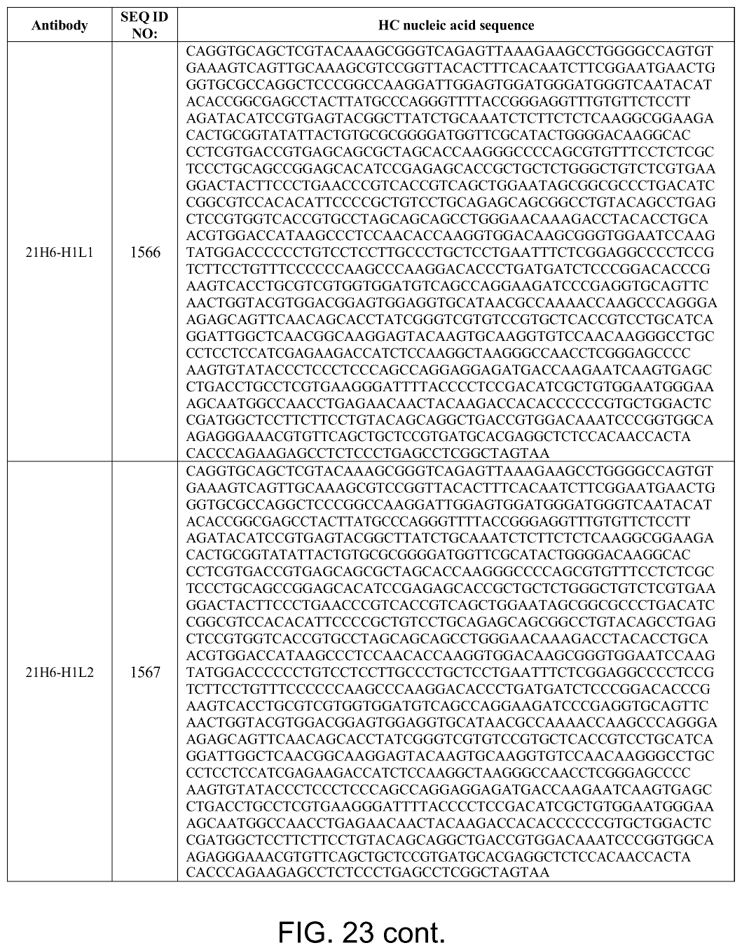

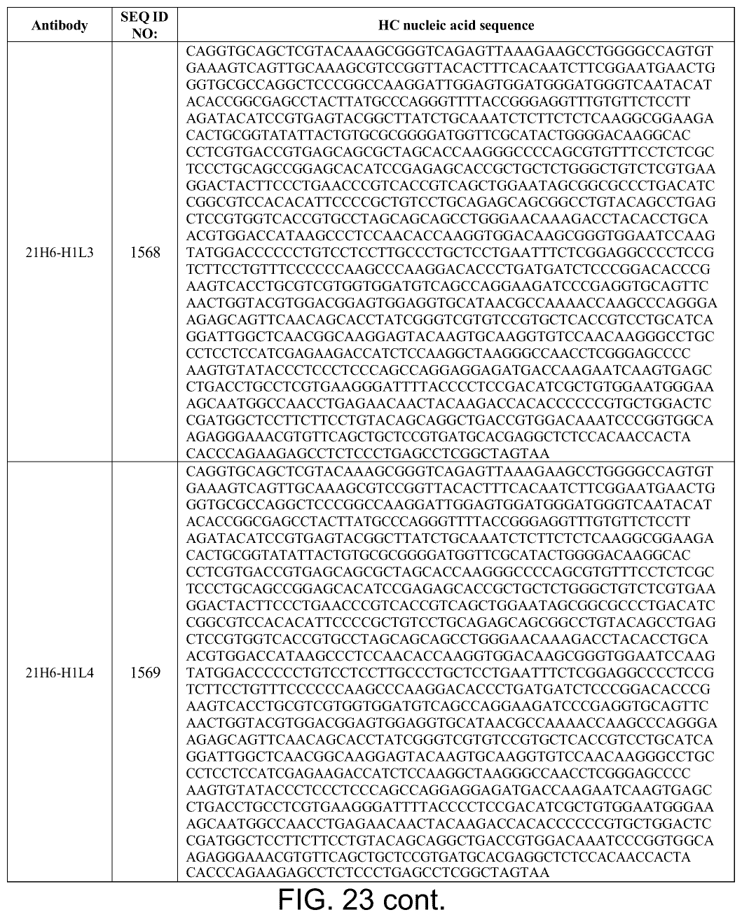

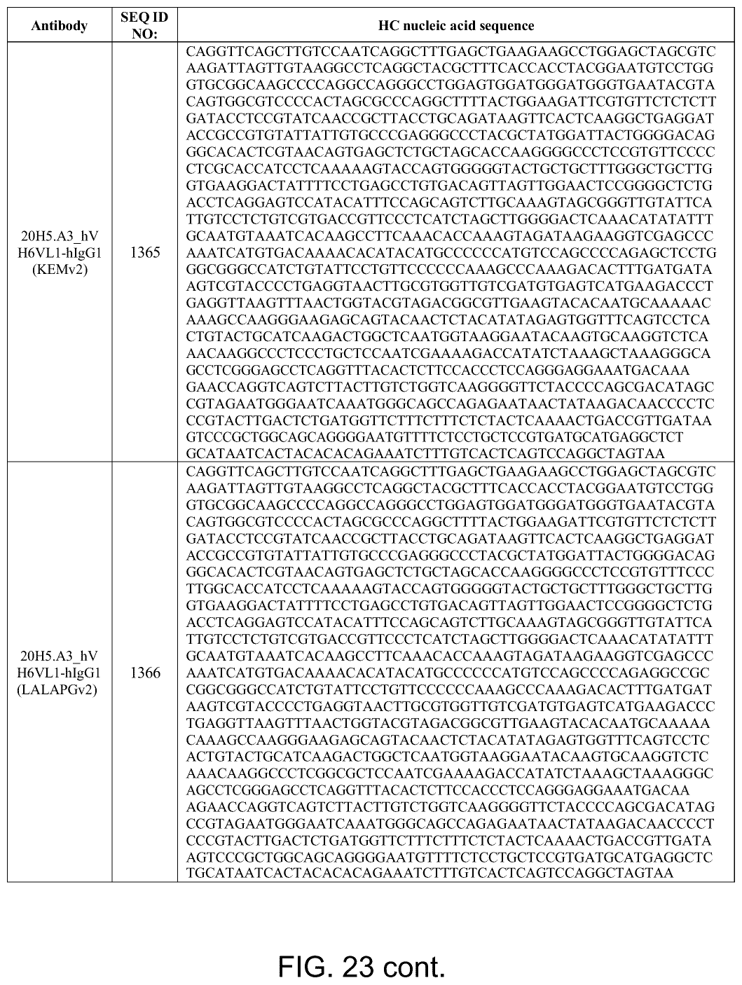

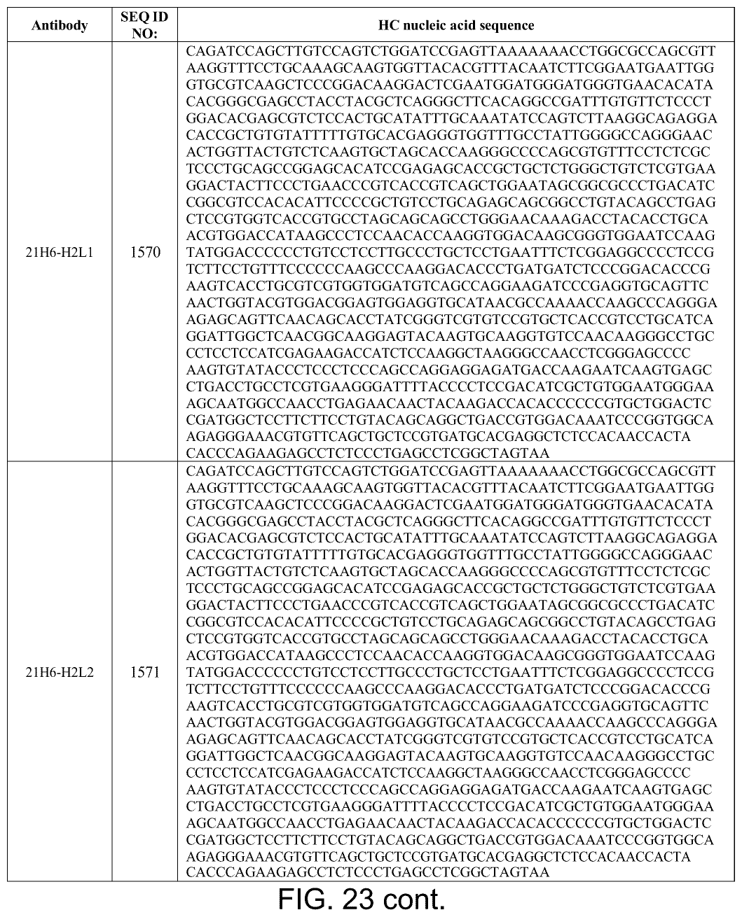

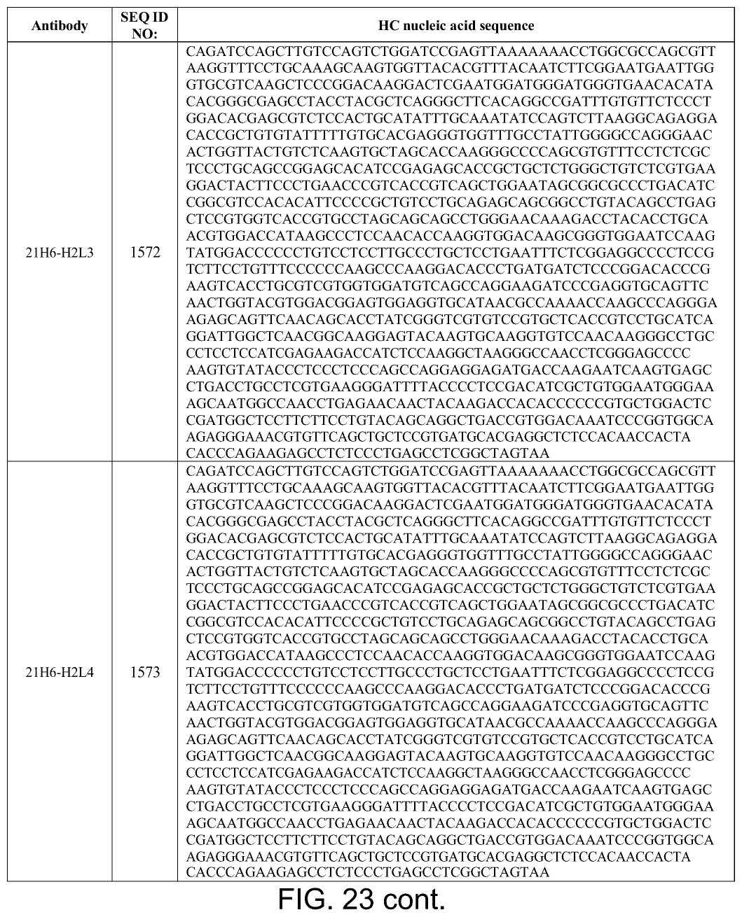

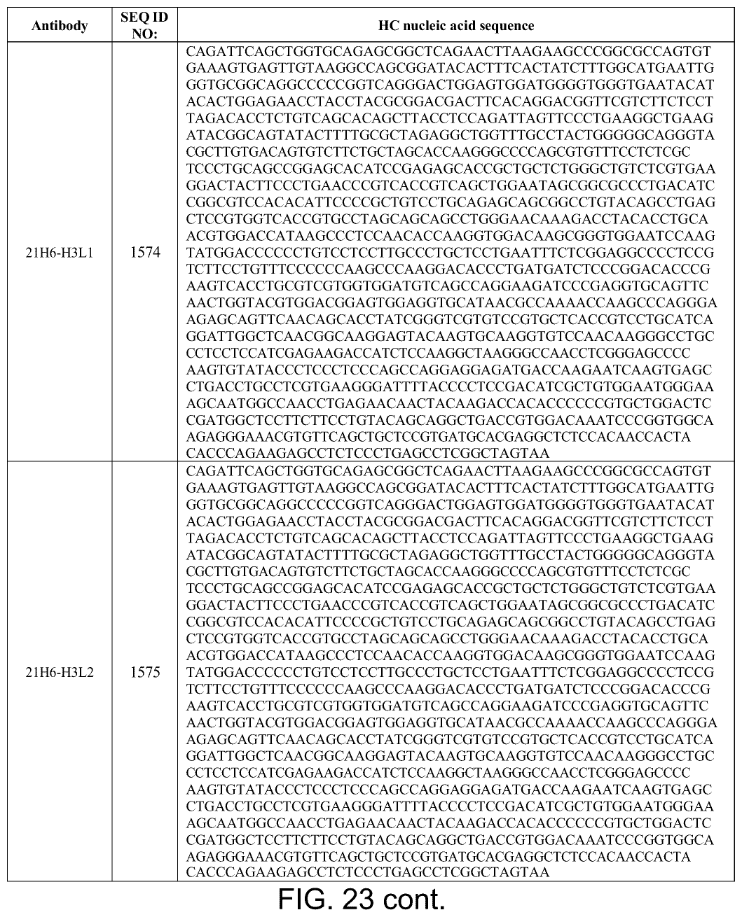

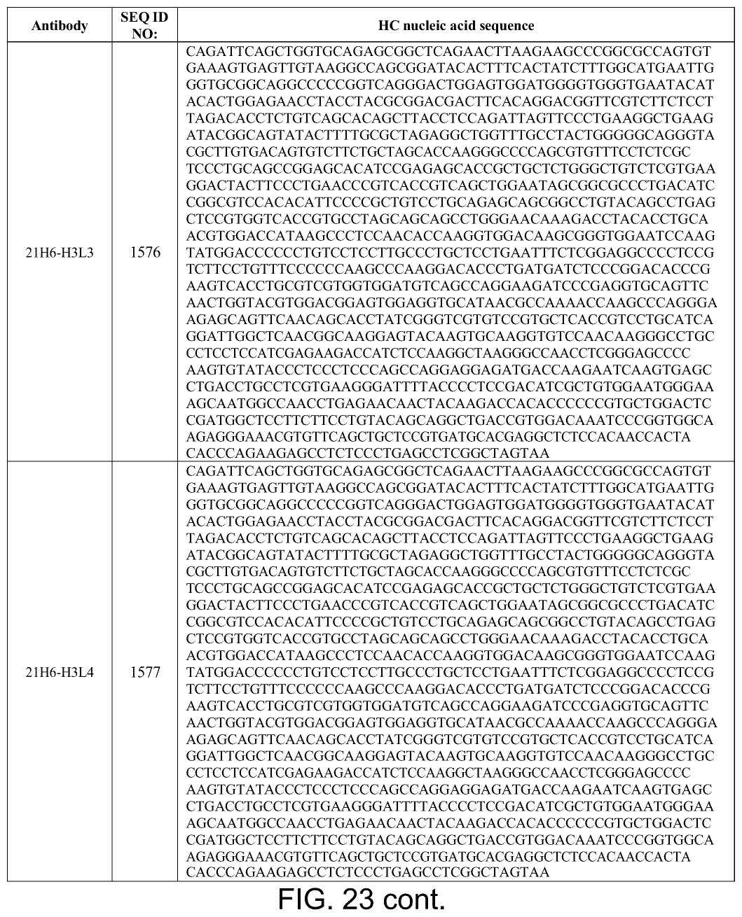

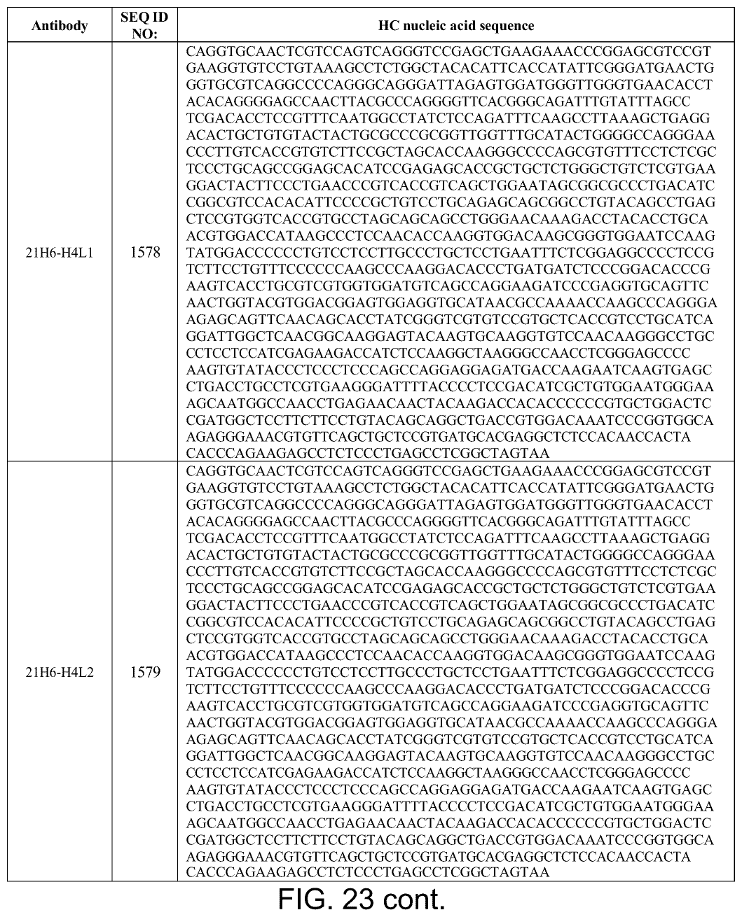

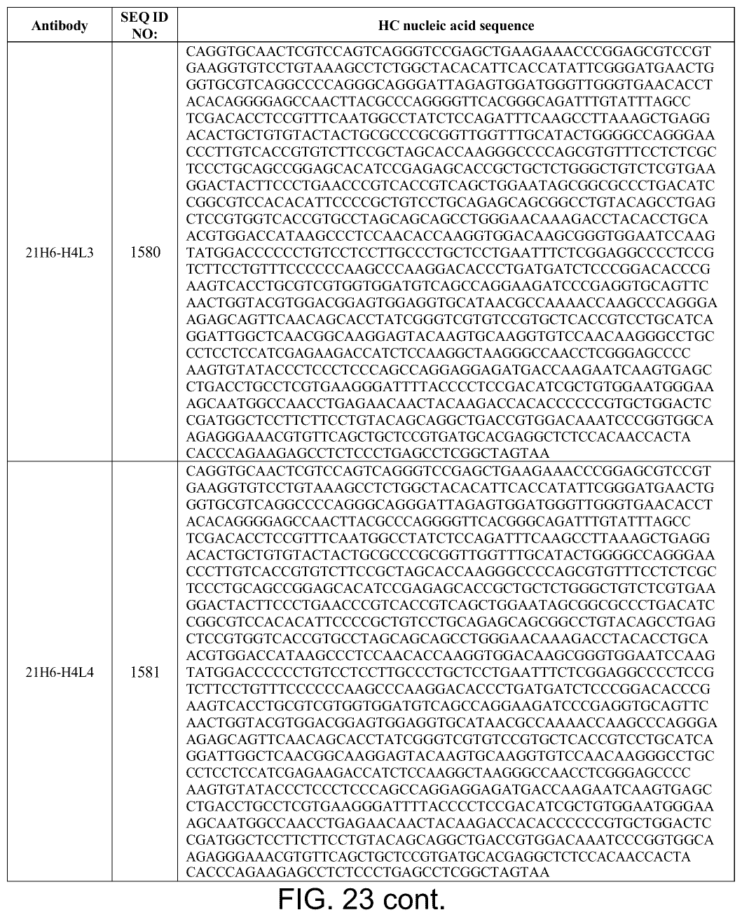

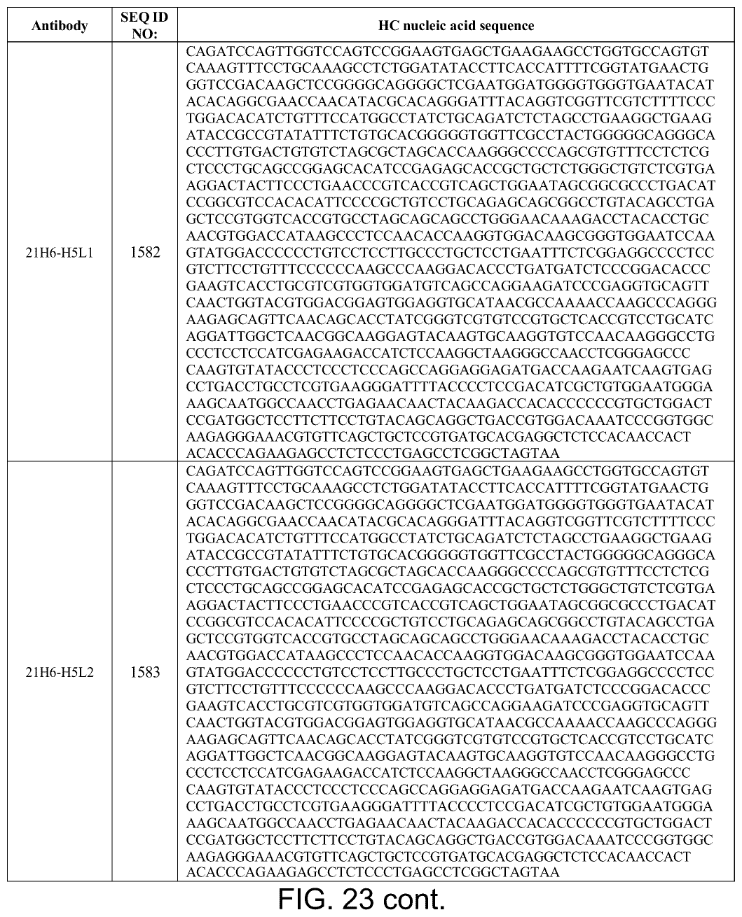

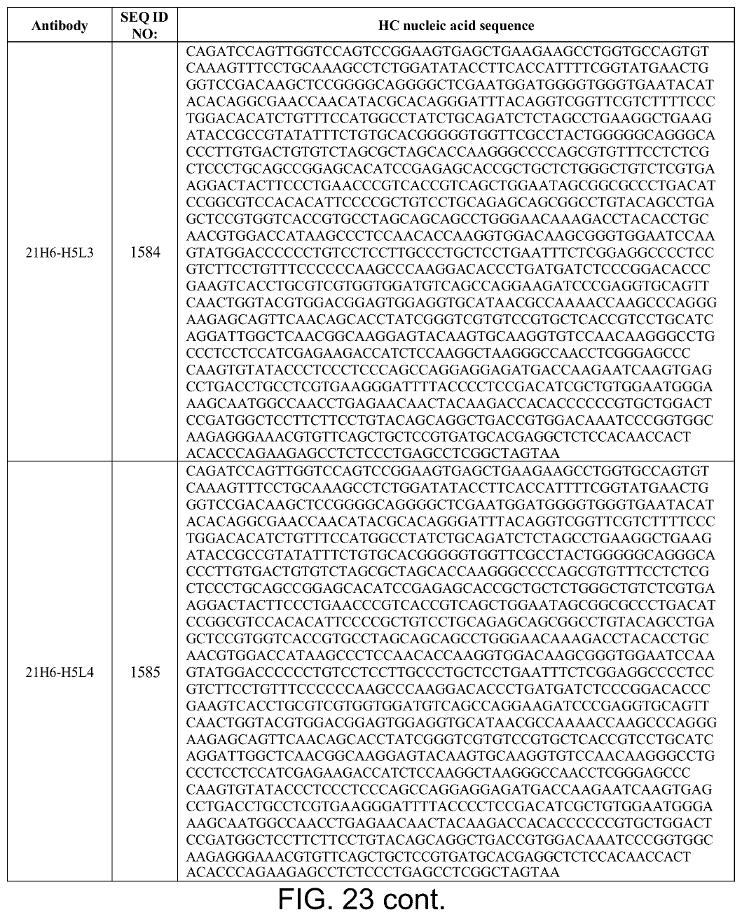

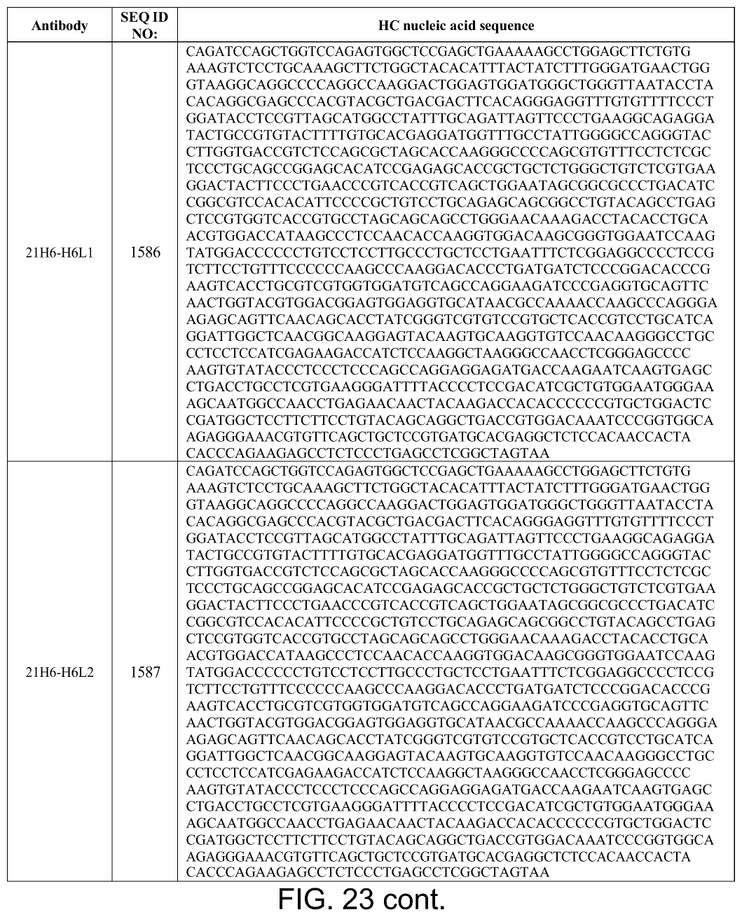

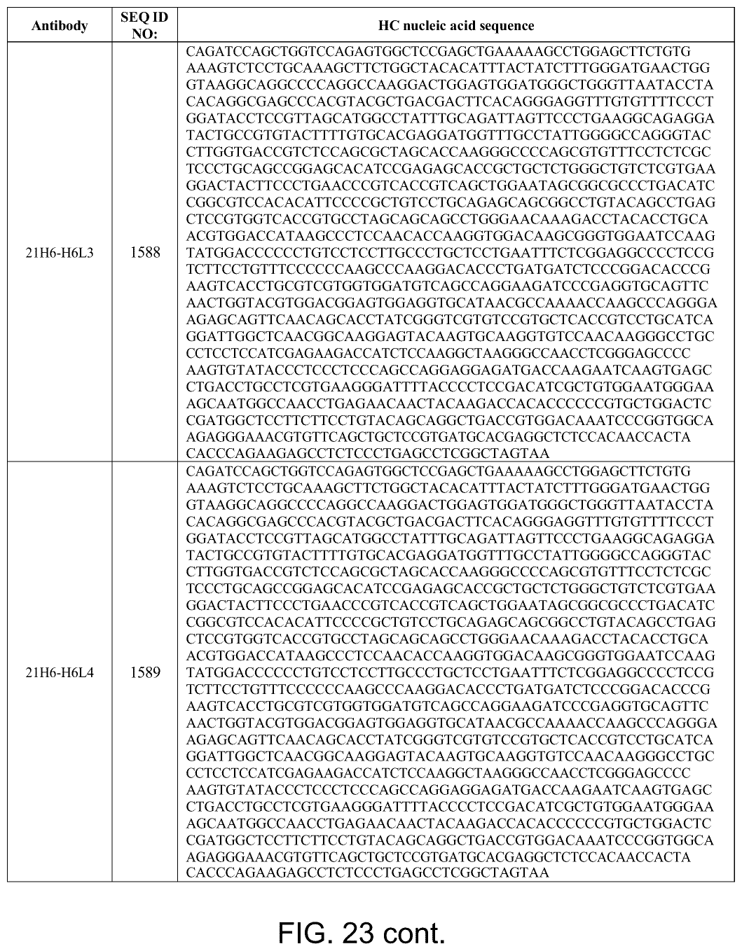

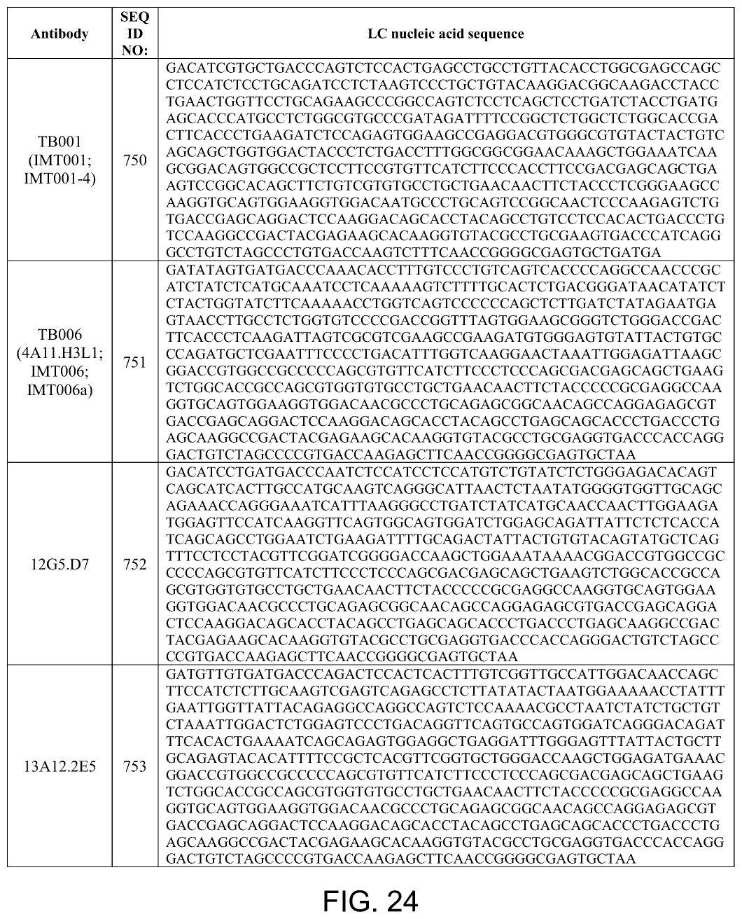

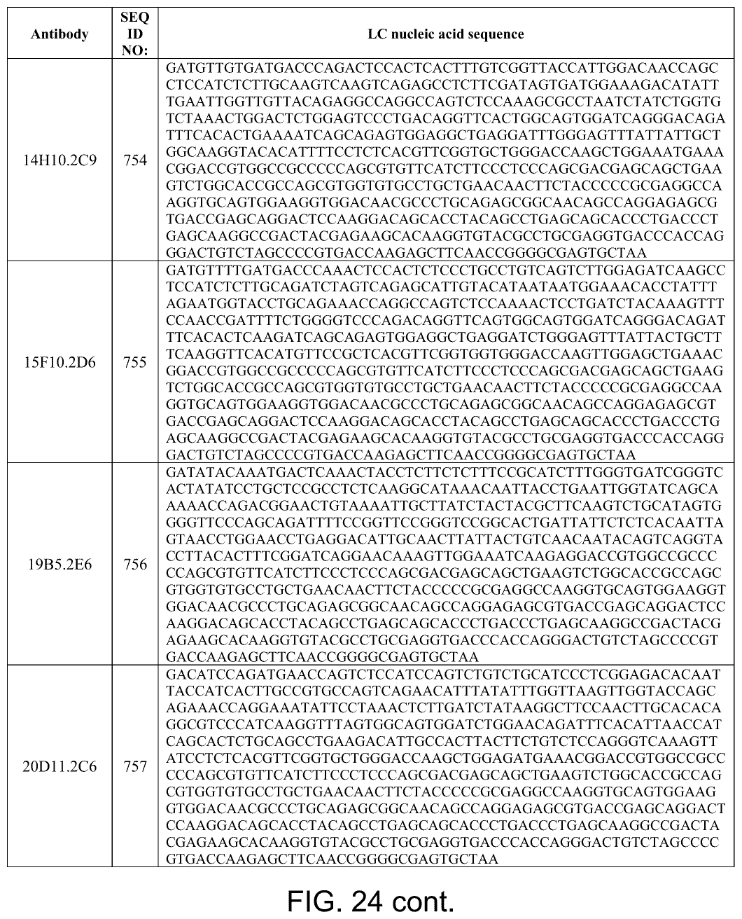

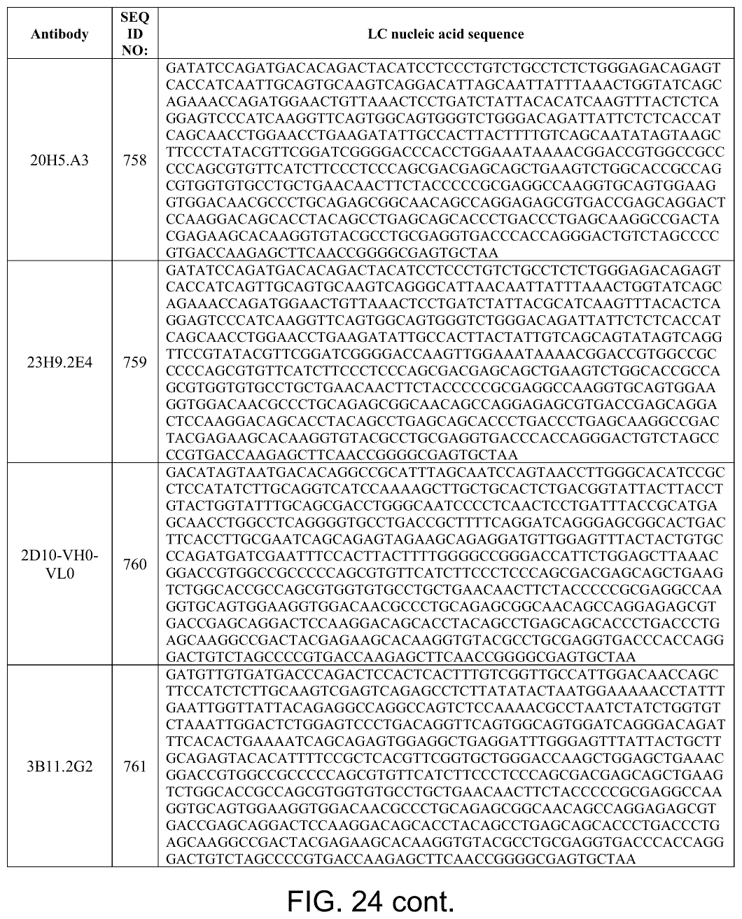

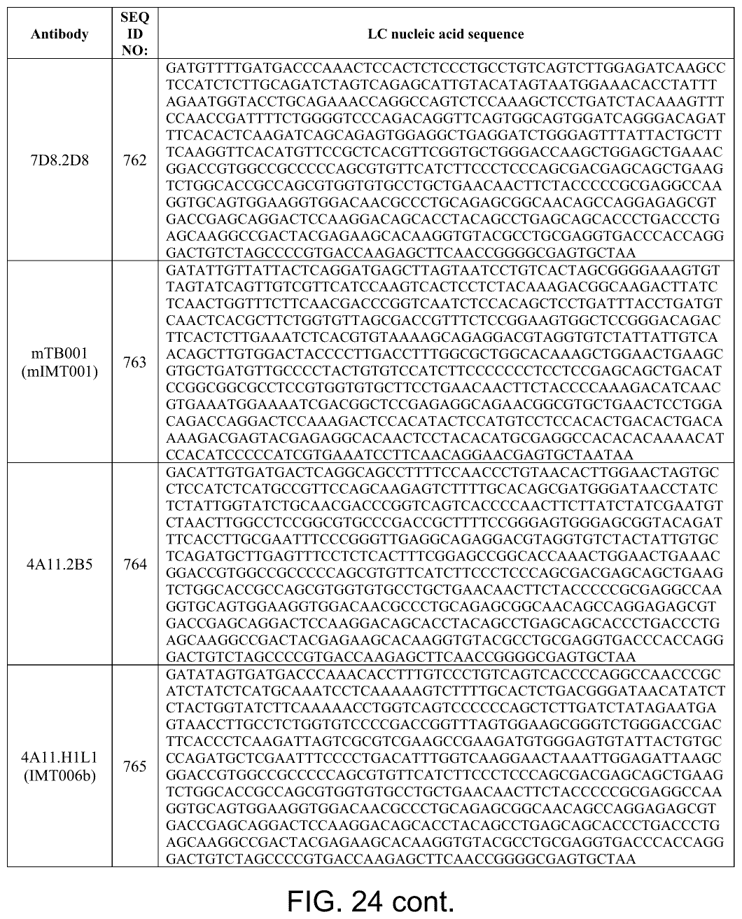

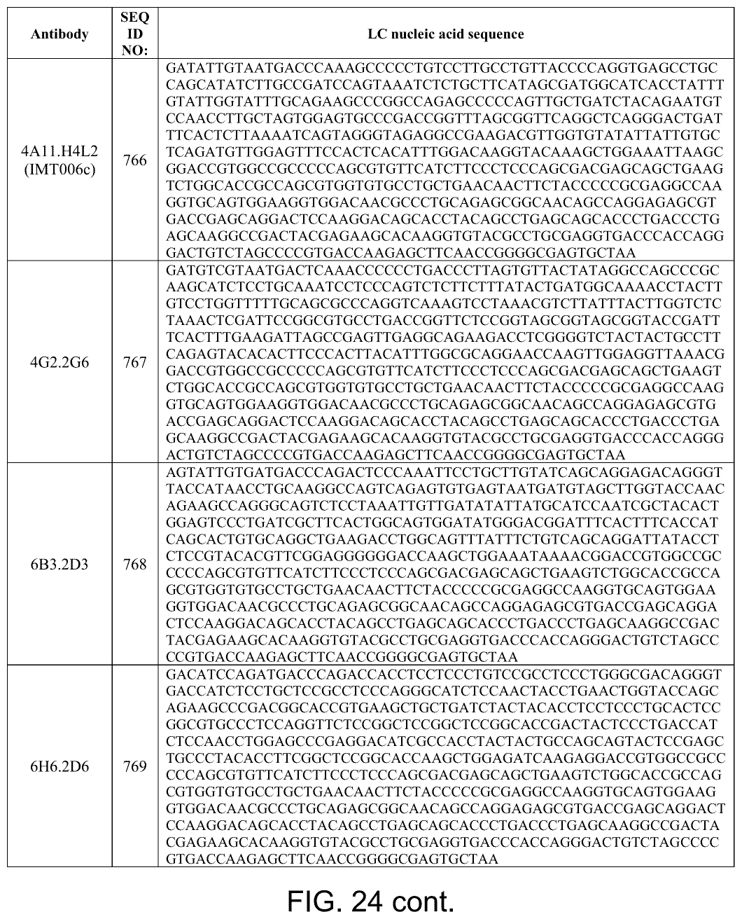

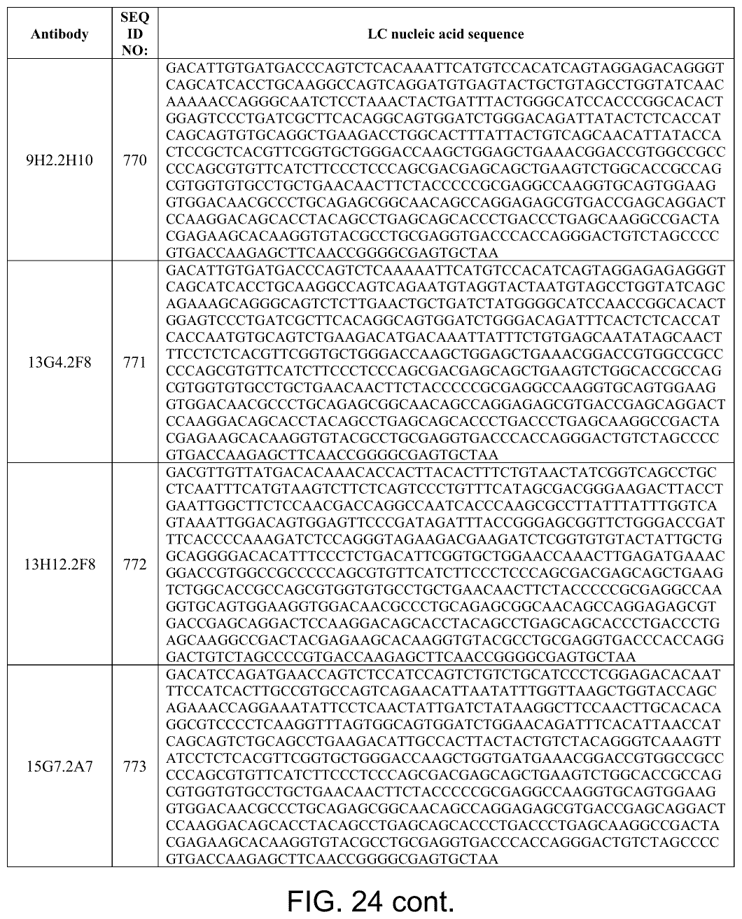

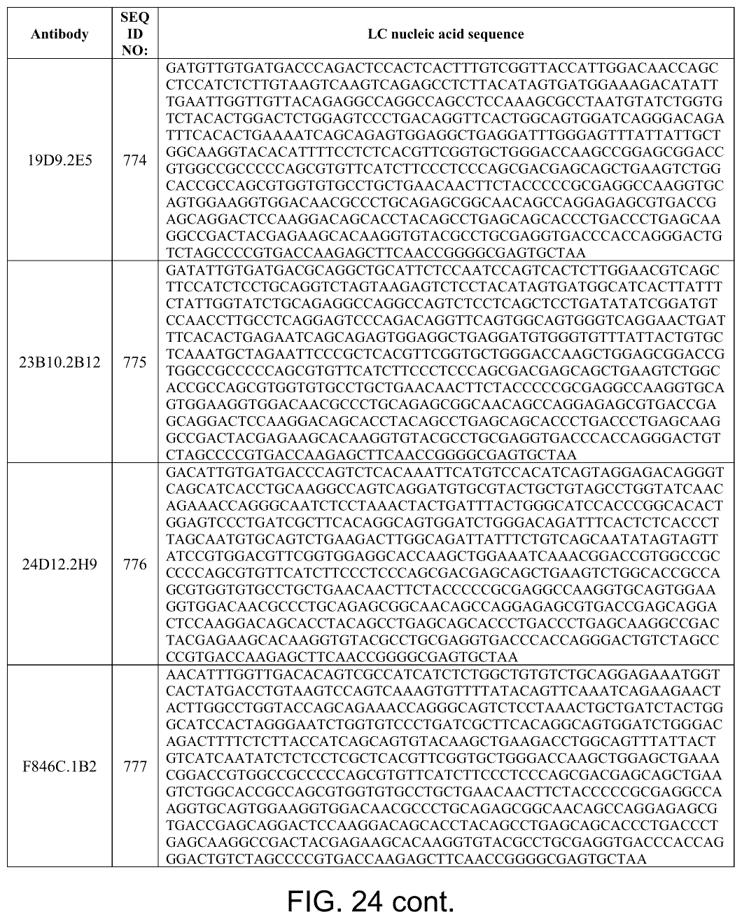

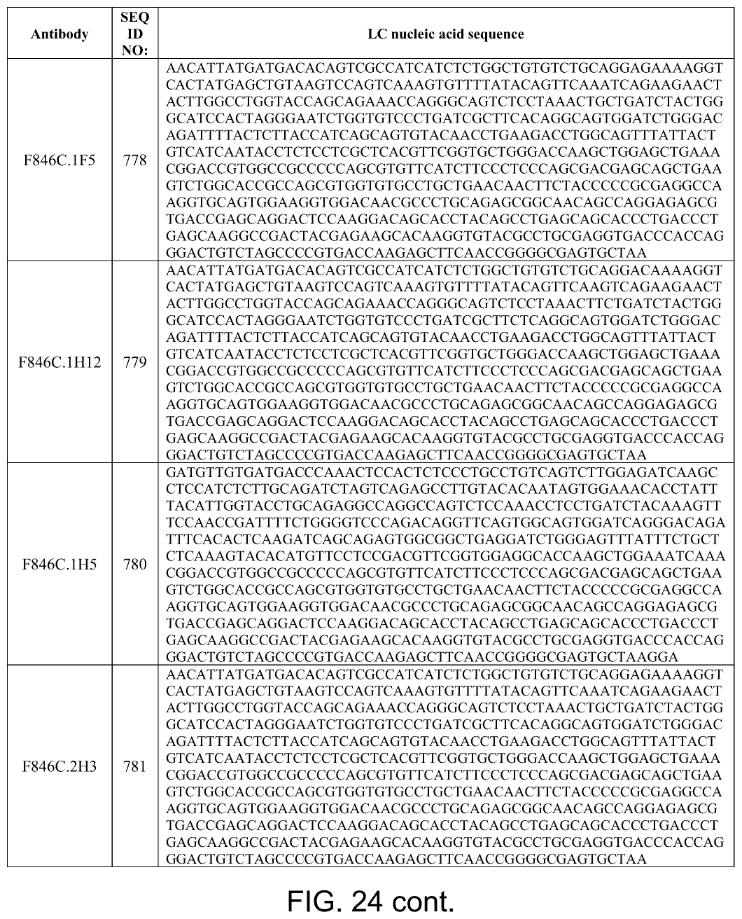

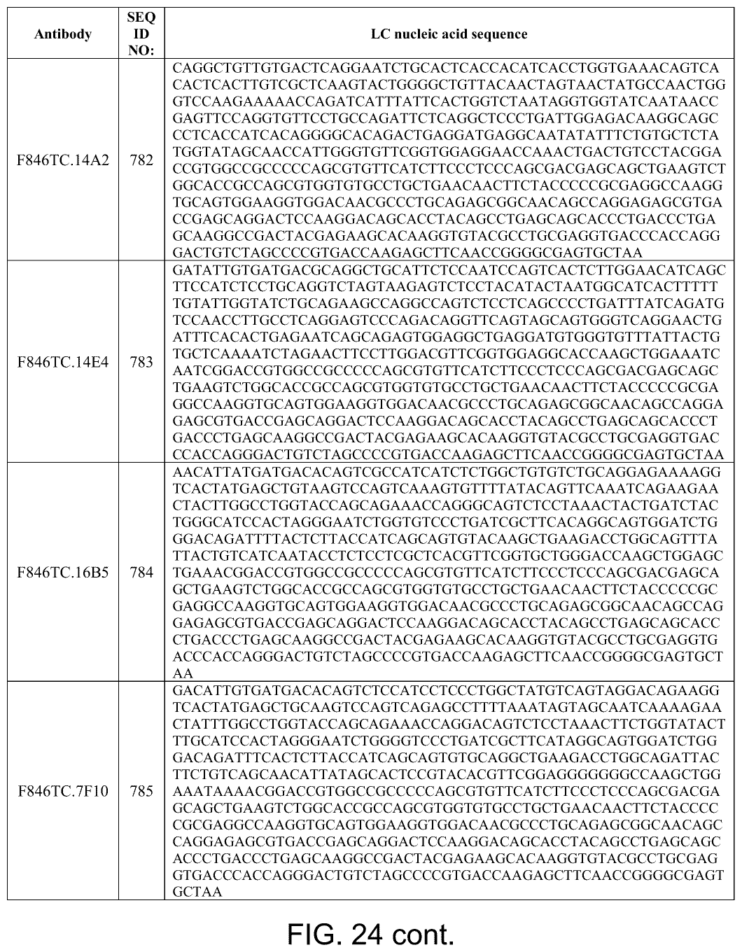

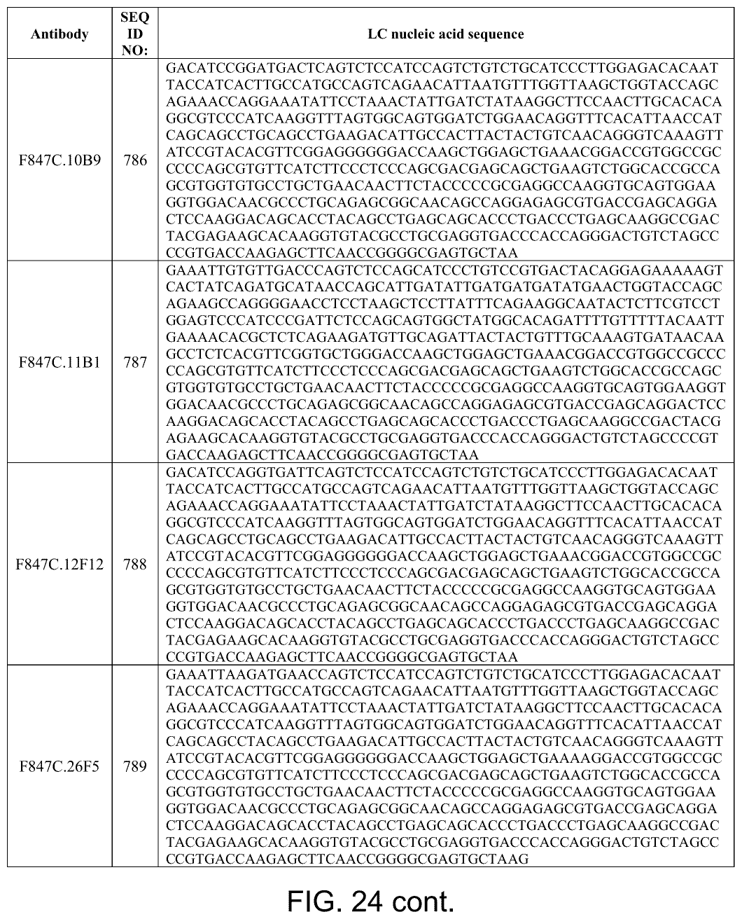

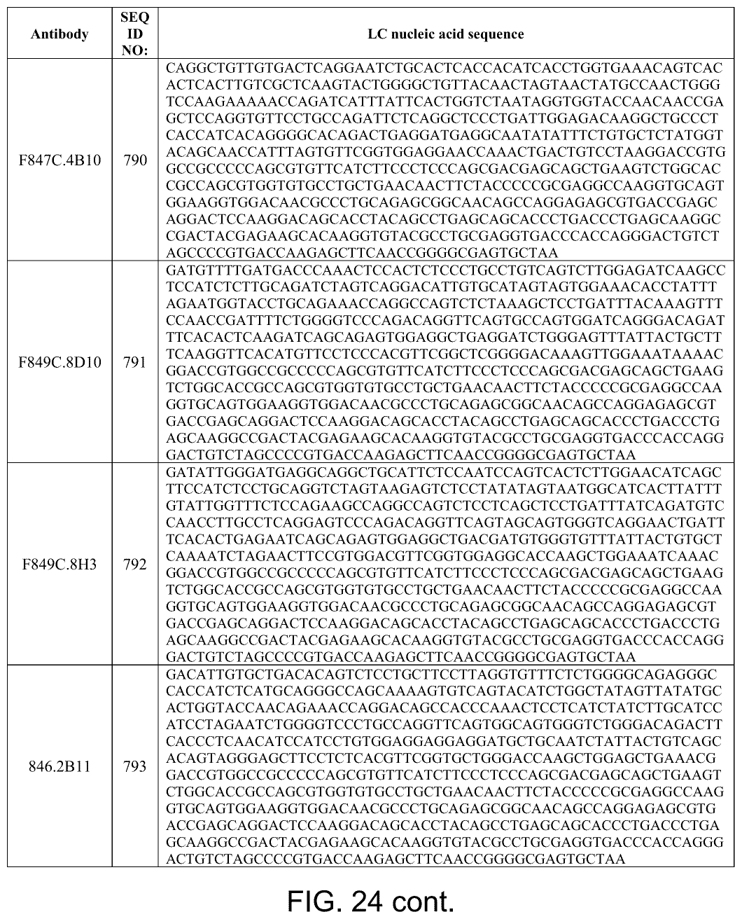

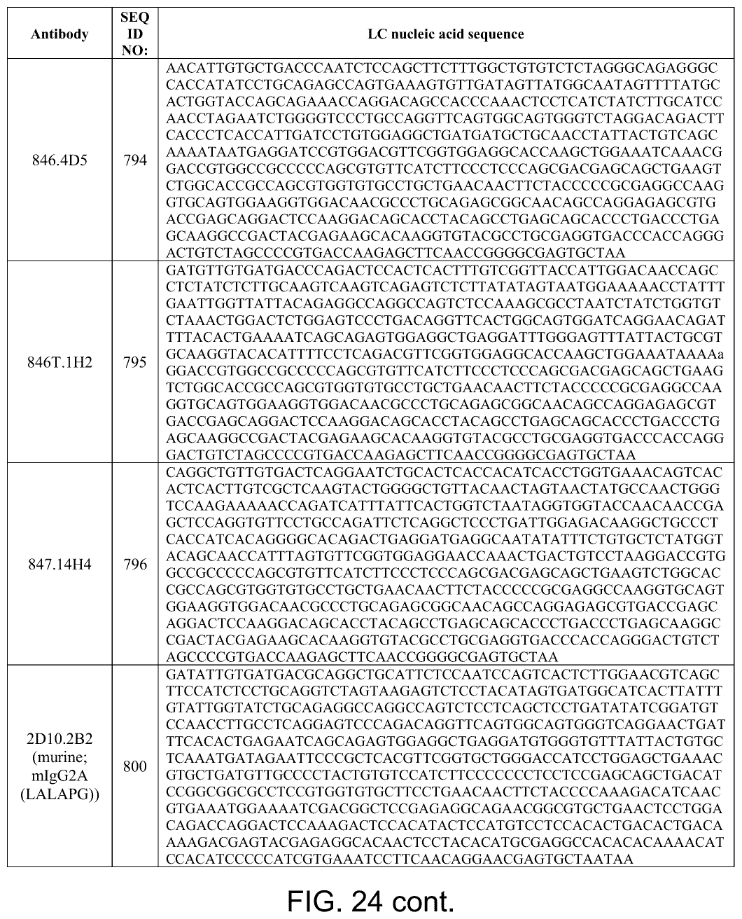

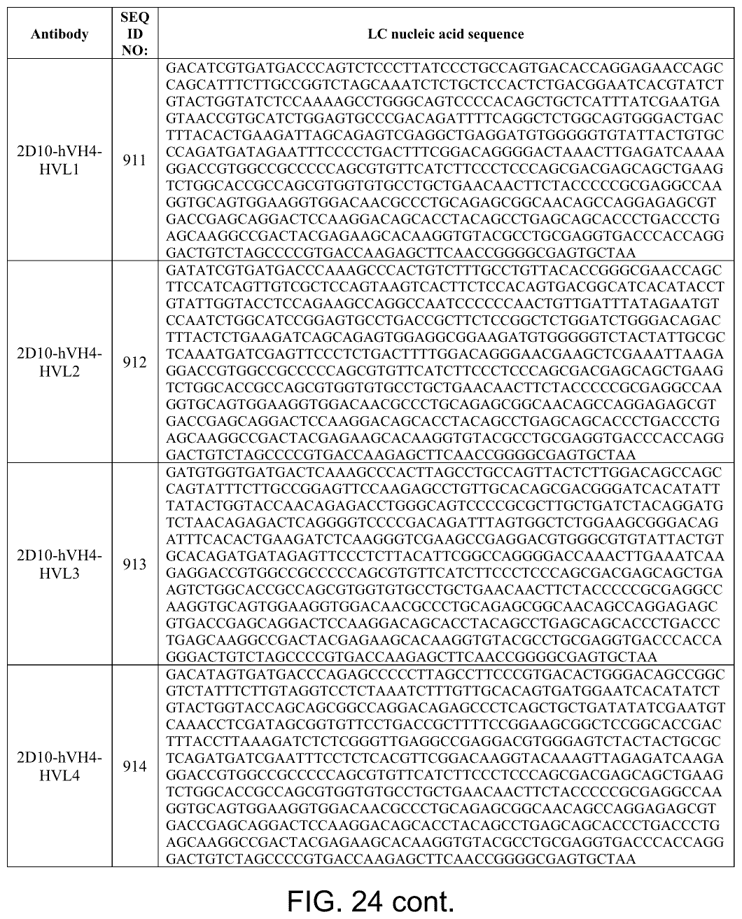

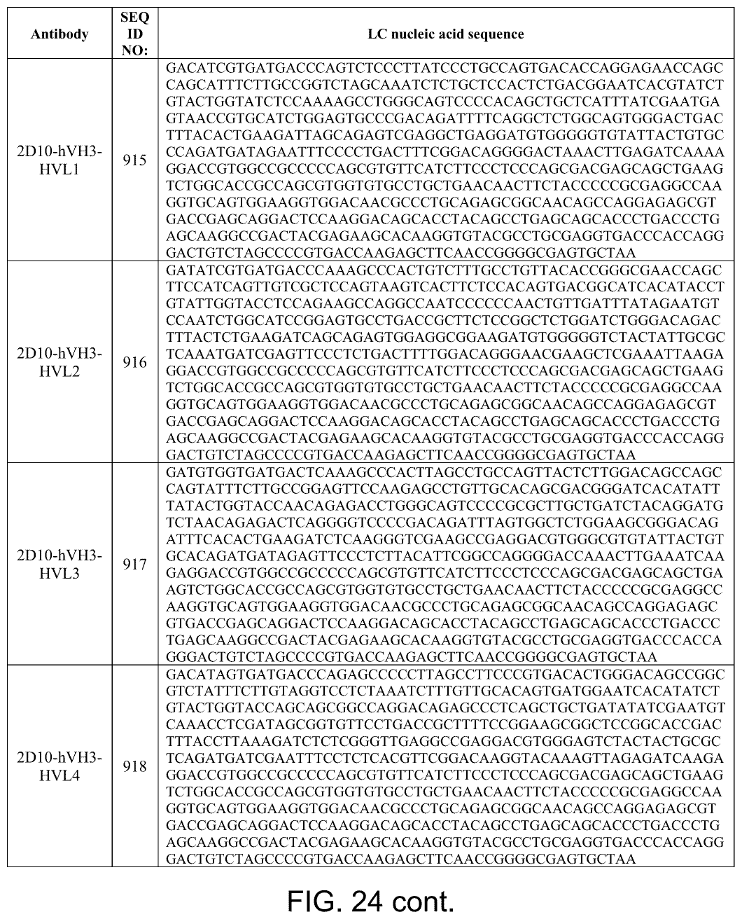

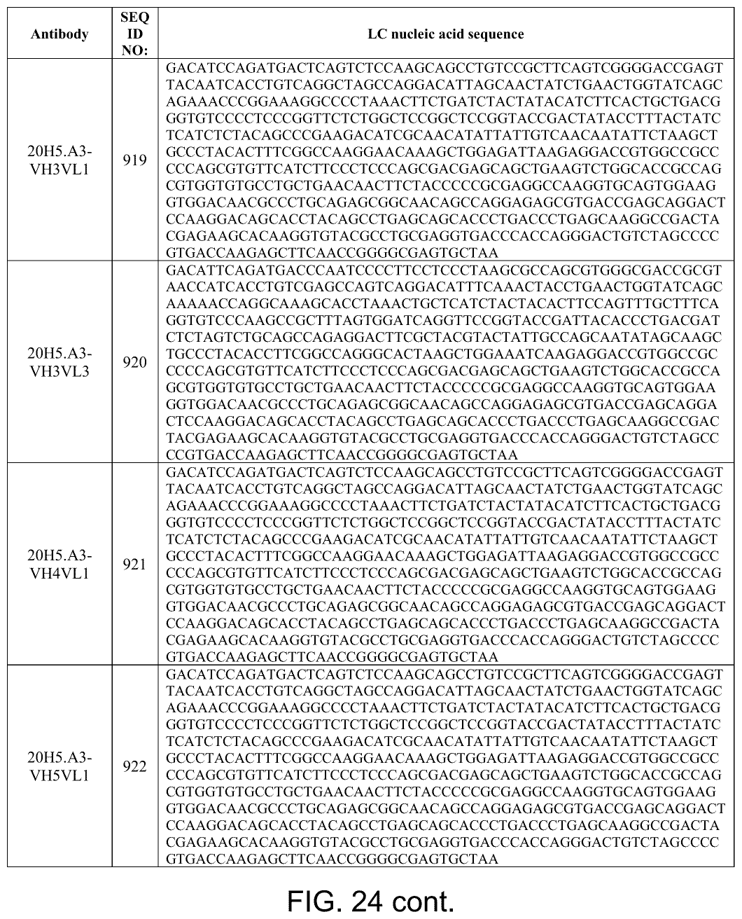

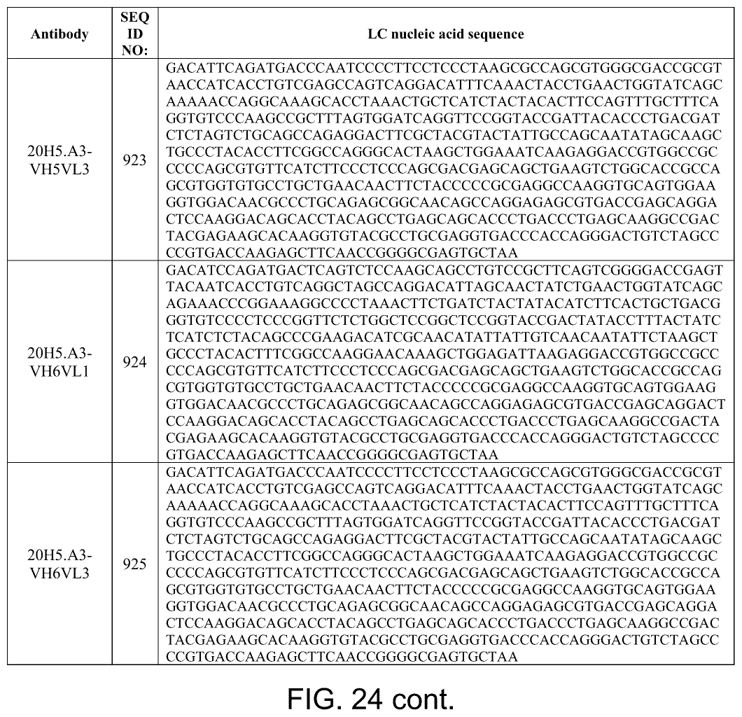

In some embodiments, nucleic acid sequences encoding for heavy chain variable regions are depicted in FIG. 21 (SEQ ID NOs: 539-620, 797, 866-880, 1011-1024, 1239-1281, 1515-1539). In some embodiments, nucleic acid sequences encoding for light chain variable regions are depicted in FIG. 22 (SEQ ID NOs: 621-702, 798, 881-895, 1025-1038, 1282-1324, 1540-1564). In some embodiments, nucleic acid sequences encoding for heavy chains are depicted in FIG. 23 (SEQ ID NO: 703-749, 799, 896-910, 1039-1052, 1325-1367, 1565-1589). In some embodiments, nucleic acid sequences encoding for light chains are depicted in FIG. 24 (SEQ ID NO: 750-796, 800, 911-925, 1053-1066, 1368-1410, 1590-1614). In some embodiments, any of the compositions or methods provided herein can include one or more of the antibody components encoded by the nucleic acids provided herein.

Any one of the anti-Gal3 antibodies or binding fragments thereof described herein can be prepared by recombinant DNA technology, synthetic chemistry techniques, or a combination thereof. For instance, sequences encoding the desired components of the anti-Gal3 antibodies, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-Gal3 antibody of interest or binding fragment thereof.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A. 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-Gal3 antibodies or binding fragments thereof can also be modified, for example, by substituting the coding sequence for human heavy and light chain constant regions in place of the homologous non-human sequences. In that manner, chimeric antibodies are prepared that retain the binding specificity of the original anti-Gal3 antibody or binding fragment thereof.

Also disclosed herein are methods of producing an anti-Gal3 antibody or binding fragment thereof. In some embodiments, the methods comprise expressing a nucleic acid that encodes for the anti-Gal3 antibody or binding fragment thereof in a cell and isolating the expressed anti-Gal3 antibody or binding fragment thereof from the cell. In some embodiments, the methods further comprise concentrating the anti-Gal3 antibody or binding fragment thereof to a desired concentration. In some embodiments, the cell is a mammalian cell, insect cell, or bacterial cell. In some embodiments, the anti-Gal3 antibody or binding fragment thereof is any one of the anti-Gal3 antibodies or binding fragments disclosed herein. Specific procedures of expressing antibodies in a cell and isolation of the expressed antibodies are conventionally known and can be practiced by one skilled in the art.

### Antibody Production

In some cases, anti-Gal3 antibodies or binding fragments thereof are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-Gal3 antibodies or binding fragments thereof can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, each incorporated fully herein by reference in its entirety. Such antibodies can be referred to as human xenogenic antibodies.

Alternatively, anti-Gal3 antibodies or binding fragments thereof can be produced from phage libraries containing human variable regions. See U.S. Pat. No. 6,174,708, incorporated fully herein by reference in its entirety.

In some aspects of any of the embodiments disclosed herein, an anti-Gal3 antibody or binding fragment thereof is produced by a hybridoma.

For monoclonal anti-Gal3 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-Gal3 antibody or binding fragment thereof may be produced by genetic engineering.

Anti-Gal3 antibodies or binding fragments thereof disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-Gal3 antibodies or binding fragments thereof include, but are not limited to, humanized, chimeric, or xenogenic human anti-Gal3 antibodies or binding fragments thereof.

Chimeric anti-Gal3 antibodies or binding fragments thereof can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains and incorporate only the complementarity-determining regions of the animal-derived antibody. This can be accomplished by carefully examining the sequence of the hyper-variable loops of the variable regions of a monoclonal antigen binding unit or monoclonal antibody and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4T S.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The disclosure thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755, each hereby incorporated by reference in its entirety.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-Gal3 antibody or binding fragment thereof can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-Gal3 antibodies or binding fragments thereof can be further classified on the basis of their binding specificities. A “monospecific” anti-Gal3 antibody or binding fragment thereof is a molecule capable of binding to one or more antigens of the same kind. A “multispecific” anti-Gal3 antibody or binding fragment thereof is a molecule having binding specificities for at least two different antigens. While such molecules normally will only bind two distinct antigens (i.e. bispecific anti-Gal3 antibodies), antibodies with additional specificities such as trispecific antibodies are encompassed by this expression when used herein. This disclosure further provides multispecific anti-Gal3 antibodies. Multispecific anti-Gal3 antibodies or binding fragments thereof are multivalent molecules capable of binding to at least two distinct antigens, e.g., bispecific and trispecific molecules exhibiting binding specificities to two and three distinct antigens, respectively.

In some embodiments, the methods further provide for screening for or identifying antibodies or binding fragments thereof capable of disrupting an interaction between Gal3 and a target protein. In some aspects, the method may comprise: (a) contacting Gal3 protein with an antibody or binding fragment thereof that selectively binds to Gal3, thereby forming a Gal3-antibody complex; (b) contacting the Gal3-antibody complex with the target protein; (c) removing unbound target protein; and (d) detecting the target protein bound to the Gal3-antibody complex, wherein the antibody or binding fragment thereof is capable of disrupting an interaction of Gal3 and the target protein when the target protein is not detected in (d). In some cases, the method comprises an immunoassay. In some cases, the immunoassay is an enzyme-linked immunosorbent assay (ELISA).

### Host Cells

In some embodiments, the present disclosure provides host cells expressing any one of the anti-Gal3 antibodies or binding fragments thereof disclosed herein. A subject host cell typically comprises a nucleic acid encoding any one of the anti-Gal3 antibodies or binding fragments thereof disclosed herein.

The disclosure provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-Gal3 antibodies or binding fragments thereof can be determined using any nucleic acid or protein assay known in the art. For example, the presence of transcribed mRNA of light chain CDRs or heavy chain CDRs, or the anti-Gal3 antibody or binding fragment thereof can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-Gal3 antibody or binding fragment thereof.

Expression of the vector can also be determined by examining the expressed anti-Gal3 antibody or binding fragment thereof. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Payload

In some embodiments, any anti-Gal3 antibody disclosed herein further comprises a payload. In some cases, the payload comprises a small molecule, a protein or functional fragment thereof, a peptide, or a nucleic acid polymer.

In some cases, the number of payloads conjugated to the anti-Gal3 antibody (e.g., the drug-to-antibody ratio or DAR) is about 1:1, one payload to one anti-Gal3 antibody. In some cases, the ratio of the payloads to the anti-Gal3 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-Gal3 antibody is about 2:1. In some cases, the ratio of the payloads to the anti-Gal3 antibody is about 3:1. In some cases, the ratio of the payloads to the anti-Gal3 antibody is about 4:1. In some cases, the ratio of the payloads to the anti-Gal3 antibody is about 6:1. In some cases, the ratio of the payloads to the anti-Gal3 antibody is about 8:1. In some cases, the ratio of the payloads to the anti-Gal3 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycine A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. As used herein, a “detectable moiety” may comprise an atom, molecule, or compound that is useful in diagnosing, detecting or visualizing a location and/or quantity of a target molecule, cell, tissue, organ, and the like. Detectable moieties that can be used in accordance with the embodiments herein include, but are not limited to, radioactive substances (e.g. radioisotopes, radionuclides, radiolabels or radiotracers), dyes, contrast agents, fluorescent compounds or molecules, bioluminescent compounds or molecules, enzyme and enhancing agents (e.g. paramagnetic ions), or specific binding moieties such as streptavidin, avidin, or biotin. In addition, some nanoparticles, for example quantum dots or metal nanoparticles can be suitable for use as a detectable moiety.

Exemplary radioactive substances that can be used as detectable moieties in accordance with the embodiments herein include, but are not limited to, ¹⁸F, ¹⁸F-FAC, ³²p, ³³p, ⁴⁵Ti, ⁴⁷Sc, ⁵²Fe, ⁵⁹Fe, ⁶²Cu, 64Cu, ⁶⁷ Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Exemplary paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as a polylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-¹⁸F complex, to a targeting molecule for use in PET analysis.

Exemplary contrast agents that can be used as detectable moieties in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable moieties in accordance with the embodiments of the disclosure include, but are not limited to, fluorescein, fluorescein isothiocyanate (FITC), OREGON GREEN™ rhodamine, Texas red, tetrarhodimine isothiocynate (TRITC), Cy3, Cy5, and the like), fluorescent markers (e.g., green fluorescent protein (GFP), phycoerythrin, and the like), autoquenched fluorescent compounds that are activated by tumor-associated proteases, enzymes (e.g., luciferase, horseradish peroxidase, alkaline phosphatase, and the like), nanoparticles, biotin, digoxigenin or combinations thereof.

Enzymes that can be used as detectable moieties in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the payload is a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles can be used as payloads to be conjugated to any one of the anti-Gal3 antibodies disclosed herein.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′—OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display enhanced properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modifications include morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof.

Any of the anti-Gal3 antibodies disclosed herein may be conjugated to one or more (e.g. 1, 2, 3, 4, 5, 6, 7, 8, 9, or 10, or more) payloads described herein.

### Conjugation Chemistry

In some instances, the payload is conjugated to an anti-Gal3 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-Gal3 antibody described herein by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-Gal3 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-Gal3 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-Gal3 antibody utilizing a microbial transglutamine catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-Gal3 antibody by a method as described in PCT Publication No. WO2014/140317, which utilizes a sequence-specific transpeptidase and is hereby expressly incorporated by reference in its entirety.

In some instances, the payload is conjugated to an anti-Gal3 antibody described herein by a method as described in U.S. Patent Publication Nos. 2015/0105539 and 2015/0105540.

### Linker

In some instances, a linker described herein comprises a natural or synthetic polymer, consisting of long chains of branched or unbranched monomers, and/or cross-linked network of monomers in two or three dimensions. In some instances, the linker includes a polysaccharide, lignin, rubber, or polyalkylen oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydisperse or monodisperse compound. In some instances, polydisperse material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (ρNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (me). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Kit/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-Gal3 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

Some embodiments provided herein are described by way of the following provided numbered arrangements A and also provided as possible combinations or overlapping embodiments:

1. A method of inhibiting Gal3-mediated amyloid aggregation of a protein, comprising:
contacting the protein with an anti-Gal3 antibody or binding fragment thereof,
wherein binding of the anti-Gal3 antibody or binding fragment thereof to Gal3 inhibits Gal3-mediated amyloid aggregation of the protein.

2. The method of arrangement 1, wherein the protein is in a cell.

3. The method of arrangement 1 or 2, wherein the method is performed in vitro or in vivo.

4. The method of any one of arrangements 1-3, wherein Gal3-mediated amyloid aggregation of the protein is inhibited by at least 5%, 10%, 15%, 20%, 25%, 30%, 35%, 40%, 45%, 50%, 55%, 60%, 65%, 70%, 75%, 80%, 85%, 90%, 95%, or 99% after contacting with the anti-Gal3 antibody or binding fragment thereof relative to a cell that is not contacted with the anti-Gal3 antibody or binding fragment thereof.

5. The method of any one of arrangements 1-4, wherein the protein comprises α-synuclein, tau protein, TAR DNA-binding protein 43 (TDP-43), transthyretin (TTR), uromodulin, islet amyloid polypeptide (IAPP), serum amyloid A (SAA), or p53, or any combination thereof.

6. The method of arrangement 5, wherein the tau protein is 4R tau and/or phosphorylated tau (phospho tau), optionally wherein the phosphorylated tau is phospho-tau (S396).

7. The method of any one of arrangements 1-6, wherein the protein comprises apolipoprotein E (APOE), prion protein, or neurofilament light (NFL), or any combination thereof, optionally wherein the apolipoprotein E is APO-E4.

8. A method of treating an amyloid proteopathy in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated amyloid aggregation of a protein in the subject, thereby treating the amyloid proteopathy in the subject.

9. The method of arrangement 8, further comprising identifying the subject as needing treatment of the amyloid proteopathy prior to the administering step.

10. The method of arrangement 8 or 9, further comprising detecting an improvement in the amyloid proteopathy in the subject following the administering step.

11. The method of arrangement 9 or 10, wherein identifying the subject as needing treatment of the amyloid proteopathy and/or detecting the improvement in the amyloid proteopathy is done by biopsy, blood or urine test, echocardiogram, or technetium pyrophosphate (99mTc-PYP) scintigraphy.

12. The method of any one of arrangements 8-11, wherein the amyloid proteopathy is reduced by at least 5%, 10%, 15%, 20%, 25%, 30%, 35%, 40%, 45%, 50%, 55%, 60%, 65%, 70%, 75%, 80%, 85%, 90%, 95%, or 99% after the administering step relative to the amyloid proteopathy prior to the administering step.

13. The method of any one of arrangements 8-12, wherein the protein comprises α-synuclein, tau protein, TAR DNA-binding protein 43 (TDP-43), transthyretin (TTR), uromodulin, islet amyloid polypeptide (IAPP), serum amyloid A (SAA), or p53, or any combination thereof.

14. The method of arrangement 13, wherein the tau protein is 4R tau and/or phosphorylated tau (phospho tau), optionally wherein the phosphorylated tau is phospho-tau (S396).

15. The method of any one of arrangement 8-14, wherein the protein comprises apolipoprotein E (APOE), prion protein, or neurofilament light (NFL), or any combination thereof, optionally wherein the apolipoprotein E is APO-E4.

16. The method of any one of arrangement 8-15, wherein the amyloid proteopathy comprises a synucleinopathy, Parkinson's disease, dementia with Lewy bodies, multiple system atrophy, tauopathy, Alzheimer's disease, progressive supranuclear palsy, corticobasal degeneration, Pick's disease, TDP-43 proteopathy, amyotrophic lateral sclerosis, frontotemporal lobar degeneration, TTR amyloidosis (ATTR), cardiac amyloidosis, uromodulin-associated kidney disease, IAPP amyloidosis, SAA amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation or any combination thereof.

17. The method of any one of arrangements 1-16, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;
the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801;
the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 112-169, 802;
the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;
the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 221-247; and
the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296.

18. The method of arrangement 17, wherein the anti-Gal3 antibody or binding fragment thereof comprises a combination of the V_{H}-CDR1, V_{H}-CDR2, V_{H}-CDR3, V_{L}-CDR1, VL-CDR2, and V_{L}-CDR3 as illustrated in FIG. 13.

19. The method of arrangements 17 or 18, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 926.

20. The method of any one of arrangements 17-19, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 927-929.

21. The method of any one of arrangements 1-20, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850.

22. The method of any one of arrangements 1-21, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865.

23. The method of any one of arrangements 1-22, wherein the anti-Gal3 antibody or binding fragment thereof is selected from the group consisting of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof.

24. A method of promoting amyloid aggregation and/or oligomerization of a protein, comprising contacting the protein with Gal3, wherein Gal3 promotes amyloid aggregation and/or oligomerization of the protein.

25. The method of arrangement 24, wherein the protein is contacted with Gal3 in an aqueous solution.

26. The method of arrangement 24 or 25, wherein Gal3 promotes amyloid aggregation and/or oligomerization of the protein on the order of 1, 2, 3, 4, 5, 6, 7, 8, 9, 10, 11, 12, 13, 14, 15, 16, 17, 18, 19, 20, 21, 22, 23, or 24 hours.

27. The method of any one of arrangements 24-26, wherein the protein comprises α-synuclein, tau protein, TDP-43, TTR, uromodulin, IAPP, SAA, or p53, or any combination thereof.

28. The method of arrangement 27, wherein the tau protein is 4R tau and/or phosphorylated tau (phospho tau), optionally wherein the phosphorylated tau is phospho-tau (S396), optionally wherein the phosphorylated tau forms trimers, tetramers, or higher order oligomers when contacted with Gal3.

29. The method of arrangement 28, wherein amyloid aggregation and/or oligomerization of the tau protein is achieved more rapidly compared to spontaneous aggregation and/or oligomerization of tau protein alone, or aggregation and/or oligomerization of tau protein when mixed with heparin and/or arachnoid acid, optionally wherein tau protein is mixed with heparin and/or arachnoid acid at 37° C. or about 37° C.

30. The method of arrangement 28 or 29, wherein amyloid aggregation and/or oligomerization of the tau protein is achieved with no more than 0.5, 1, 2, 3, 4, 5, 6, 7, 8, 9, 10, 11, 12, 13, 14, 15, 16, 17, 18, 19, 20, 21, 22, 23, or 24 hours of contacting the tau protein with Gal3.

31. The method of any one of arrangements 24-30, wherein the protein comprises APOE, prion protein, or NFL, or any combination thereof, optionally wherein the APOE is APO-E4.

32. The method of any one of arrangements 24-31, wherein the protein is contacted with Gal3 at room temperature.

33. A composition comprising a protein and Gal3, wherein Gal3 promotes amyloid aggregation and/or oligomerization of the protein.

34. The composition of arrangement 33, wherein the protein and Gal3 are in aqueous solution, or are dried and/or lyophilized.

35. The composition of arrangement 33 or 34, wherein the protein comprises α-synuclein, tau protein, TDP-43, TTR, uromodulin, IAPP, SAA, or p53, or any combination thereof.

36. The composition of arrangement 35, wherein the tau protein is 4R tau and/or phosphorylated tau (phospho tau), optionally wherein the phosphorylated tau is phospho-tau (5396), optionally wherein the phosphorylated tau forms trimers, tetramers, or higher order oligomers when contacted with Gal3.

37. The composition of any one of arrangements 33-36, wherein the protein comprises APOE, prion protein, or NFL, or any combination thereof, optionally wherein the APOE is APO-E4

38. A kit comprising the composition of any one of arrangements 33-37.

Some embodiments provided herein are described by way of the following provided numbered arrangements B and also provided as possible combinations or overlapping embodiments:

1. A method of inhibiting Gal3-mediated amyloid aggregation of a protein, comprising:
contacting the protein with an anti-Gal3 antibody or binding fragment thereof,
wherein binding of the anti-Gal3 antibody or binding fragment thereof to Gal3 inhibits Gal3-mediated amyloid aggregation of the protein.

2. A method of inhibiting Gal3-mediated oligomerization of a protein, comprising:
contacting the protein with an anti-Gal3 antibody or binding fragment thereof,
wherein binding of the anti-Gal3 antibody or binding fragment thereof to Gal3 inhibits Gal3-mediated oligomerization of the protein.

3. The method of any one of the preceding arrangements, wherein the protein is in a cell.

4. The method of any one of the preceding arrangements, wherein the method is performed in vitro or in vivo.

5. The method of any one of the preceding arrangements, wherein Gal3-mediated amyloid aggregation or oligomerization of the protein is inhibited by at least 5%, 10%, 15%, 20%, 25%, 30%, 35%, 40%, 45%, 50%, 55%, 60%, 65%, 70%, 75%, 80%, 85%, 90%, 95%, or 99% after contacting with the anti-Gal3 antibody or binding fragment thereof relative to a cell that is not contacted with the anti-Gal3 antibody or binding fragment thereof.

6. The method of any one of the preceding arrangements, wherein the protein comprises α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, and/or any combination thereof.

7. A method of treating an amyloid proteopathy in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated amyloid aggregation of a protein in the subject, thereby treating the amyloid proteopathy in the subject.

8. A method of treating a proteopathy in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of a protein in the subject, thereby treating the amyloid proteopathy in the subject.

9. The method of any one of the preceding arrangements, further comprising identifying the subject as needing treatment of the proteopathy and/or amyloid proteopathy prior to the administering step.

10. The method of any one of the preceding arrangements, further comprising detecting an improvement in the proteopathy and/or amyloid proteopathy in the subject following the administering step.

11. The method of any one of the preceding arrangements, wherein identifying the subject as needing treatment of the proteopathy and/or amyloid proteopathy and/or detecting the improvement in the amyloid proteopathy is done by biopsy, blood or urine test, echocardiogram, or technetium pyrophosphate (99mTc-PYP) scintigraphy.

12. The method of any one of the preceding arrangements, wherein the proteopathy and/or amyloid proteopathy is reduced by at least 5%, 10%, 15%, 20%, 25%, 30%, 35%, 40%, 45%, 50%, 55%, 60%, 65%, 70%, 75%, 80%, 85%, 90%, 95%, or 99% after the administering step relative to the amyloid proteopathy prior to the administering step.

13. The method of any one of the preceding arrangements, wherein the protein comprises α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, and/or any combination thereof.

14. The method of any one of the preceding arrangements, wherein the proteopathy and/or amyloid proteopathy comprises a synucleinopathy, Parkinson's disease, dementia with Lewy bodies, multiple system atrophy, tauopathy, Alzheimer's disease, progressive supranuclear palsy, corticobasal degeneration, Pick's disease, TDP-43 proteopathy, amyotrophic lateral sclerosis, frontotemporal lobar degeneration, TTR amyloidosis (ATTR), cardiac amyloidosis, uromodulin-associated kidney disease, IAPP amyloidosis, SAA amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation or any combination thereof.

15. The method of any one of the preceding arrangements, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;
the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952;
the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NO: 112-169, 802, 953, 954;
the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;
the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 211-247; and
the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

16. The method of any one of the preceding arrangements, wherein the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 13; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

17. The method of any one of the preceding arrangements, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 940, 955-968, 1067-1109, 1415-1439; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

18. The method of any one of the preceding arrangements, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

19. The method of any one of the preceding arrangements, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

20. The method of any one of the preceding arrangements, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

21. The method of any one of the preceding arrangements, wherein the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

22. The method of any one of the preceding arrangements, wherein the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

23. The method of any one of the preceding arrangements, wherein the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

24. A method of promoting amyloid aggregation and/or oligomerization of a protein, comprising contacting the protein with Gal3, wherein Gal3 promotes amyloid aggregation and/or oligomerization of the protein.

25. The method of any one of the preceding arrangements, wherein the protein is contacted with Gal3 in an aqueous solution.

26. The method of any one of the preceding arrangements, wherein Gal3 promotes amyloid aggregation and/or oligomerization of the protein on the order of 1, 2, 3, 4, 5, 6, 7, 8, 9, 10, 11, 12, 13, 14, 15, 16, 17, 18, 19, 20, 21, 22, 23, or 24 hours.

27. The method of any one of the preceding arrangements, wherein the protein comprises α-synuclein, tau protein, TDP-43, TTR, uromodulin, IAPP, SAA, or p53, or any combination thereof.

28. The method of any one of the preceding arrangements, wherein the tau protein is 4R tau and/or phosphorylated tau (phospho tau), optionally wherein the phosphorylated tau is phospho-tau (S396), optionally wherein the phosphorylated tau forms trimers, tetramers, or higher order oligomers when contacted with Gal3.

29. The method of any one of the preceding arrangements, wherein amyloid aggregation and/or oligomerization of the tau protein is achieved more rapidly compared to spontaneous aggregation and/or oligomerization of tau protein alone, or aggregation and/or oligomerization of tau protein when mixed with heparin and/or arachnoid acid, optionally wherein tau protein is mixed with heparin and/or arachnoid acid at 37° C. or about 37° C.

30. The method of any one of the preceding arrangements, wherein amyloid aggregation and/or oligomerization of the tau protein is achieved with no more than 0.5, 1, 2, 3, 4, 5, 6, 7, 8, 9, 10, 11, 12, 13, 14, 15, 16, 17, 18, 19, 20, 21, 22, 23, or 24 hours of contacting the tau protein with Gal3.

31. The method of any one of the preceding arrangements, wherein the protein comprises APOE, prion protein, or NFL, or any combination thereof, optionally wherein the APOE is APO-E4.

32. The method of any one of the preceding arrangements, wherein the protein is contacted with Gal3 at room temperature.

33. A composition comprising a protein and Gal3, wherein Gal3 promotes amyloid aggregation and/or oligomerization of the protein.

34. The composition of any one of the preceding arrangements, wherein the protein and Gal3 are in aqueous solution, or are dried and/or lyophilized.

35. The composition of any one of the preceding arrangements, wherein the protein comprises α-synuclein, tau protein, TDP-43, TTR, uromodulin, IAPP, SAA, or p53, or any combination thereof.

36. The composition of any one of the preceding arrangements, wherein the tau protein is 4R tau and/or phosphorylated tau (phospho tau), optionally wherein the phosphorylated tau is phospho-tau (S396), optionally wherein the phosphorylated tau forms trimers, tetramers, or higher order oligomers when contacted with Gal3.

37. The composition of any one of the preceding arrangements, wherein the protein comprises APOE, prion protein, or NFL, or any combination thereof, optionally wherein the APOE is APO-E4

38. A kit comprising the composition of any one of any one of the preceding arrangements.

39. A method of inhibiting Gal3-mediated amyloid aggregation of amyloid β40 and/or amyloid β42, comprising:
contacting the protein with an anti-Gal3 antibody or binding fragment thereof,
wherein binding of the anti-Gal3 antibody or binding fragment thereof to Gal3 inhibits Gal3-mediated amyloid aggregation of amyloid β40 and/or amyloid p42.

40. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated amyloid aggregation of amyloid β40 and/or amyloid p42 in the subject, thereby treating Alzheimer's disease in the subject.

41. A method of treating CAA in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated aggregation of amyloid β40 and/or amyloid p42 in the subject, thereby treating CAA in the subject.

42. A method of inhibiting Gal3-mediated amyloid aggregation of phospho tau, comprising:
contacting the protein with an anti-Gal3 antibody or binding fragment thereof,
wherein binding of the anti-Gal3 antibody or binding fragment thereof to Gal3 inhibits Gal3-mediated oligomerization of phospho tau.

43. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of phospho tau in the subject, thereby treating Alzheimer's disease in the subject.

44. A method of treating tauopathies in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of phospho tau in the subject, thereby treating the tauopathy in the subject.

45. A method of inhibiting Gal3-mediated oligomerization of alpha synuclein, comprising:
contacting the protein with an anti-Gal3 antibody or binding fragment thereof,
wherein binding of the anti-Gal3 antibody or binding fragment thereof to Gal3 inhibits Gal3-mediated oligomerization of alpha synuclein.

46. A method of treating Lewy body disease in a subject in need thereof, comprising: administering to the subject an anti-Gal3 antibody or binding fragment thereof,
wherein binding of the anti-Gal3 antibody or binding fragment thereof to Gal3 in the
subject inhibits Gal3-mediated oligomerization of alpha synuclein in the subject, thereby treating Lewy body disease in the subject.

47. A method of treating multiple system atrophy in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of alpha synuclein in the subject, thereby treating multiple system atrophy in the subject.

48. A method of inhibiting Gal3-mediated oligomerization of APOE-4, comprising:
contacting the protein with an anti-Gal3 antibody or binding fragment thereof,
wherein binding of the anti-Gal3 antibody or binding fragment thereof to Gal3 inhibits Gal3-mediated oligomerization of APOE-4.

49. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of APOE-4 in the subject, thereby treating Alzheimer's disease in the subject.

50. A method of treating CAA in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of alpha synuclein in the subject, thereby treating CAA in the subject.

51. A method of inhibiting Gal3-mediated oligomerization of cholesterol, comprising: contacting the protein with an anti-Gal3 antibody or binding fragment thereof, wherein binding of the anti-Gal3 antibody or binding fragment thereof to Gal3 inhibits Gal3-mediated oligomerization of cholesterol.

52. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cholesterol in the subject, thereby treating Alzheimer's disease in the subject.

53. A method of treating cardiovascular disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cholesterol in the subject, thereby treating cardiovascular disease in the subject.

54. A method of treating atherosclerosis disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cholesterol in the subject, thereby treating atherosclerosis in the subject.

55. A method of inhibiting Gal3-mediated oligomerization of cholesteryl, comprising:
contacting the protein with an anti-Gal3 antibody or binding fragment thereof,
wherein binding of the anti-Gal3 antibody or binding fragment thereof to Gal3 inhibits Gal3-mediated oligomerization of cholesteryl.

56. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cholesteryl in the subject, thereby treating Alzheimer's disease in the subject.

57. A method of treating cardiovascular disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cholesteryl in the subject, thereby treating cardiovascular disease in the subject.

58. A method of treating atherosclerosis disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cholesteryl in the subject, thereby treating atherosclerosis in the subject.

59. A method of inhibiting Gal3-mediated oligomerization of neuroserpin, comprising:
contacting the protein with an anti-Gal3 antibody or binding fragment thereof,
wherein binding of the anti-Gal3 antibody or binding fragment thereof to Gal3 inhibits Gal3-mediated oligomerization of neuroserpin.

60. A method of treating familial encephalopathy with neuroserpin inclusion bodies (FENIB) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of neuroserpin in the subject, thereby treating familial encephalopathy with neuroserpin inclusion bodies (FENIB) in the subject.

61. A method of inhibiting Gal3-mediated oligomerization of insulin, comprising:
contacting the protein with an anti-Gal3 antibody or binding fragment thereof,
wherein binding of the anti-Gal3 antibody or binding fragment thereof to Gal3 inhibits Gal3-mediated oligomerization of insulin.

62. A method of treating insulin-derived amyloidosis in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of insulin in the subject, thereby treating insulin-derived amyloidosis in the subject.

63. A method of treating diabetes in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of insulin in the subject, thereby treating diabetes in the subject.

64. A method of inhibiting Gal3-mediated oligomerization of cystatin-c, comprising:
contacting the protein with an anti-Gal3 antibody or binding fragment thereof,
wherein binding of the anti-Gal3 antibody or binding fragment thereof to Gal3 inhibits Gal3-mediated oligomerization of cystatin-c.

65. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cystatin-c in the subject, thereby treating Alzheimer's disease in the subject.

66. A method of treating CAA in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cystatin-c in the subject, thereby treating CAA in the subject.

67. A method of treating kidney disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of cystatin-c in the subject, thereby treating kidney disease in the subject.

68. A method of inhibiting Gal3-mediated oligomerization of prion protein, comprising:
contacting the protein with an anti-Gal3 antibody or binding fragment thereof,
wherein binding of the anti-Gal3 antibody or binding fragment thereof to Gal3 inhibits Gal3-mediated oligomerization of prion protein.

69. A method of treating prion disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of prion protein in the subject, thereby treating prion disease in the subject.

70. A method of treating transmissible spongiform encephalopathy (TSE) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of prion protein in the subject, thereby treating transmissible spongiform encephalopathy (TSE) in the subject.

71. A method of treating familial Creutzfeldt-Jakob disease (CJD) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of prion protein in the subject, thereby treating familial Creutzfeldt-Jakob disease (CJD) in the subject.

72. A method of treating fatal familial insomnia in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of prion protein in the subject, thereby treating fatal familial insomnia in the subject.

73. A method of treating Gerstmann-Straussler-Scheinker disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of prion protein in the subject, thereby treating Gerstmann-Straussler-Scheinker disease in the subject.

74. A method of inhibiting Gal3-mediated oligomerization of myostatin, comprising:
contacting the protein with an anti-Gal3 antibody or binding fragment thereof,
wherein binding of the anti-Gal3 antibody or binding fragment thereof to Gal3 inhibits Gal3-mediated oligomerization of myostatin.

75. A method of treating idiopathic inflammatory myopathies (IIM) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of myostatin in the subject, thereby treating idiopathic inflammatory myopathies (IIM) in the subject.

76. A method of inhibiting Gal3-mediated oligomerization of transthyretin, comprising:
contacting the protein with an anti-Gal3 antibody or binding fragment thereof,
wherein binding of the anti-Gal3 antibody or binding fragment thereof to Gal3 inhibits Gal3-mediated oligomerization of transthyretin.

77. A method of treating transthyretin amyloidosis in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of transthyretin in the subject, thereby treating transthyretin amyloidosis in the subject.

78. A method of treating heart and/or kidney disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of transthyretin in the subject, thereby treating heart and/or kidney disease in the subject.

79. A method of treating preeclampsia in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of transthyretin in the subject, thereby treating preeclampsia in the subject.

80. A method of inhibiting Gal3-mediated oligomerization of phenylalanine, comprising:
contacting the protein with an anti-Gal3 antibody or binding fragment thereof,
wherein binding of the anti-Gal3 antibody or binding fragment thereof to Gal3 inhibits Gal3-mediated oligomerization of phenylalanine.

81. A method of treating phenylketonuria in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of phenylalanine in the subject, thereby treating phenylketonuria in the subject.

82. A method of inhibiting Gal3-mediated oligomerization of glutamine, comprising:
contacting the protein with an anti-Gal3 antibody or binding fragment thereof,
wherein binding of the anti-Gal3 antibody or binding fragment thereof to Gal3 inhibits Gal3-mediated oligomerization of glutamine.

83. A method of treating Huntington Disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of glutamine in the subject, thereby treating Huntington Disease in the subject.

84. A method of inhibiting Gal3-mediated oligomerization of Neurofibrillary Light chain (NFL), comprising:
contacting the protein with an anti-Gal3 antibody or binding fragment thereof,
wherein binding of the anti-Gal3 antibody or binding fragment thereof to Gal3 inhibits Gal3-mediated oligomerization of NFL.

85. A method of treating motor neuron degeneration in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of NFL in the subject, thereby treating motor neuron degeneration in the subject.

86. A method of inhibiting Gal3-mediated amyloid aggregation of fibrin, comprising:
contacting the protein with an anti-Gal3 antibody or binding fragment thereof,
wherein binding of the anti-Gal3 antibody or binding fragment thereof to Gal3 inhibits Gal3-mediated oligomerization of fibrin.

87. A method of treating cerebrovascular damage in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of fibrin in the subject, thereby treating cerebrovascular damage in the subject.

88. A method of treating stroke in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of fibrin in the subject, thereby treating stroke in the subject.

89. A method of treating CAA in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of fibrin in the subject, thereby treating CAA in the subject.

90. A method of treating Alzheimer's disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of fibrin in the subject, thereby treating Alzheimer's disease in the subject.

91. A method of inhibiting Gal3-mediated oligomerization of lysozyme, comprising:
contacting the protein with an anti-Gal3 antibody or binding fragment thereof,
wherein binding of the anti-Gal3 antibody or binding fragment thereof to Gal3 inhibits Gal3-mediated oligomerization of lysozyme.

92. A method of treating human systemic amyloid disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of lysozyme in the subject, thereby treating human systemic amyloid disease in the subject.

93. A method of inhibiting Gal3-mediated amyloid aggregation of complement proteins C3 and/or C9, comprising:
contacting the protein with an anti-Gal3 antibody or binding fragment thereof,
wherein binding of the anti-Gal3 antibody or binding fragment thereof to Gal3 inhibits Gal3-mediated oligomerization of complement proteins C3 and/or C9.

94. A method of treating disruption in innate immune system in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of complement proteins C3 and/or C9 in the subject, thereby treating disruption in innate immune system in the subject.

95. A method of inhibiting Gal3-mediated oligomerization of crystallins, comprising:
contacting the protein with an anti-Gal3 antibody or binding fragment thereof,
wherein binding of the anti-Gal3 antibody or binding fragment thereof to Gal3 inhibits Gal3-mediated oligomerization of crystallins.

96. A method of treating damage to lens of a subject's eye and/or blurring of vision in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of crystallins in the subject, thereby treating damage to lenses of the subject's eye and/or blurring of vision in the subject.

97. A method of inhibiting Gal3-mediated oligomerization of atrial natriuretic peptide (ANP), comprising:
contacting the protein with an anti-Gal3 antibody or binding fragment thereof,
wherein binding of the anti-Gal3 antibody or binding fragment thereof to Gal3 inhibits Gal3-mediated oligomerization of ANP.

98. A method of treating congestive heart failure (CHF) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of ANP in the subject, thereby treating CHF in the subject.

99. A method of treating cardiac amyloidosis in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of ANP in the subject, thereby treating cardiac amyloidosis in the subject.

100. A method of inhibiting Gal3-mediated oligomerization of B-Type Natriuretic Peptide (BNP), comprising:
contacting the protein with an anti-Gal3 antibody or binding fragment thereof,
wherein binding of the anti-Gal3 antibody or binding fragment thereof to Gal3 inhibits Gal3-mediated oligomerization of BNP.

101. A method of treating congestive heart failure (CHF) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of BNP in the subject, thereby treating CHF in the subject.

102. A method of treating cardiac amyloidosis in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of BNP in the subject, thereby treating cardiac amyloidosis in the subject.

103. A method of inhibiting Gal3-mediated oligomerization calcitonin, comprising:
contacting the protein with an anti-Gal3 antibody or binding fragment thereof,
wherein binding of the anti-Gal3 antibody or binding fragment thereof to Gal3 inhibits Gal3-mediated amyloid aggregation of calcitonin.

104. A method of treating medullary carcinoma of the thyroid (MTC) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of calcitonin in the subject, thereby treating MTC in the subject.

105. A method of treating osteoporosis in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of calcitonin in the subject, thereby treating osteoporosis in the subject.

106. A method of treating Paget's Disease in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of calcitonin in the subject, thereby treating Paget's Disease in the subject.

107. A method of inhibiting Gal3-mediated oligomerization of Serum Amyloid (A) (SAA), comprising:
contacting the protein with an anti-Gal3 antibody or binding fragment thereof,
wherein binding of the anti-Gal3 antibody or binding fragment thereof to Gal3 inhibits Gal3-mediated oligomerization of Serum Amyloid (A) (SAA).

108. A method of treating peripheral amyloidosis in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of Serum Amyloid (A) (SAA) in the subject, thereby treating peripheral amyloidosis in the subject.

109. A method of inhibiting Gal3-mediated amyloid aggregation of islet amyloid polypeptide (IAPP), comprising:
contacting the protein with an anti-Gal3 antibody or binding fragment thereof,
wherein binding of the anti-Gal3 antibody or binding fragment thereof to Gal3 inhibits Gal3-mediated oligomerization of IAPP.

110. A method of treating type 2 diabetes in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of IAPP in the subject, thereby treating type 2 diabetes in the subject.

111. A method of inhibiting Gal3-mediated amyloid aggregation of TAR DNA binding protein 43 (TDP-43), comprising:
contacting the protein with an anti-Gal3 antibody or binding fragment thereof,
wherein binding of the anti-Gal3 antibody or binding fragment thereof to Gal3 inhibits Gal3-mediated oligomerization of TDP-43.

112. A method of treating amyotrophic lateral sclerosis (ALS) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of TDP-43 in the subject, thereby treating ALS in the subject.

113. A method of treating frontotemporal lobar degeneration (FTLD) in a subject in need thereof, comprising:
administering to the subject an anti-Gal3 antibody or binding fragment thereof,
wherein binding of the anti-Gal3 antibody or binding fragment thereof to Gal3 in the subject inhibits Gal3-mediated oligomerization of TDP-43 in the subject, thereby treating FTLD in the subject.

114. The method of any one of the preceding arrangements, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;
the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952;
the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NO: 112-169, 802, 953, 954;
the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;
the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 211-247; and
the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

115. The method of any one of the preceding arrangements, wherein the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 13; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

116. The method of any one of the preceding arrangements, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 940, 955-968, 1067-1109, 1415-1439; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

117. The method of any one of the preceding arrangements, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

118. The method of any one of the preceding arrangements, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

119. The method of any one of the preceding arrangements, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

120. The method of any one of the preceding arrangements, wherein the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VH5VL1, 20H5.A3-VH5VL3, 20H5.A3-VH6VL1, 20H5.A3-VH6VL3, or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

121. The method of any one of the preceding arrangements, wherein the anti-Gal3 antibody or binding fragment thereof is selected from the group consisting of at least one of: 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

122. The method of any one of the preceding arrangements, wherein the anti-Gal3 antibody or binding fragment thereof is selected from the group consisting of at least one of: 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4, or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

123. The method of any one of the preceding arrangements, wherein 80% is determined by a competition assay as provided in Example 54.

124. The method of any one of the preceding arrangements, wherein 80% is determined as follows:
A) Ab diluted 2-fold in PBS from a concentration of 4 μg/ml and coated a 96-well ELISA plate by adding 80 μl per well;
B) After incubating the plate at 4° C. overnight, the plate is washed with 300 μl PBST three times, followed by a blocking step with 150 μl of 2% BSA in PBST per well and incubated for an hour at room temperature (RT) with gentle rocking;
C) Binding solutions are prepared by 2-fold dilutions from 4 μg/ml in a 2% buffer of BSA in PBST to a concentration of 4 μg/ml;
D) The dilution is then applied to the plate by adding 60 μl per well column-wise for each galectin-3, then serially diluted two-fold length-wise in 2% BSA in PBST;
E) The plate is incubated for an hour at RT with gentle rocking, then washed with 300 μl PBST three times;
F) Afterwards, HRP-tagged anti-FLAG antibodies are diluted to 1:2000 in 2% BSA in PBST, and 25 μl is added to all the wells
G) The plate is incubated for 40 minutes at RT with gentle rocking, then washed with 300 μl PBST three times;
H) To develop the plate, 50 μl of ABTS substrate is added to each well and incubated until a sufficiently high signal was achieved;
I) The plate is read in a plate reader at an absorbance of 405 nm; and optionally,
J) Data can be graphed using GraphPad Prism 8.0 software (GraphPad Software Inc).

125. A method of inhibiting Gal3-mediated oligomerization, comprising:
contacting one or more monomers with an anti-Gal3 antibody or binding fragment thereof,
wherein binding of the anti-Gal3 antibody or binding fragment thereof to Gal3 inhibits Gal3-mediated oligomerization of the pone or more monomers.

### EXAMPLES

Some aspects of the embodiments discussed above are disclosed in further detail in the following examples, which are not in any way intended to limit the scope of the present disclosure. Those skilled in the art will appreciate that many other embodiments also fall within the scope of the invention, as it is described herein above and in the claims.

### Example 1. Gal3 Antibodies Block Gal3-Induced Aggregation of α-Synuclein

The pathological aggregation of α-Synuclein is a common feature of synucleinopathies including Parkinson's disease (PD), dementia with Lewy bodies and multiple system atrophy. To test whether anti-Gal3 antibodies could be utilized to inhibit synucleinopathies, it was first established that Gal3 promotes α-Synuclein aggregation. α-Synuclein was mixed with recombinant human Gal3 and the molecular weight of complexes were measured with Western blot.

The α-Synuclein protein (R&D Systems Cat #SP-485-500) was diluted to a final concentration of 0.1 mg/ml by adding 10 mM sodium phosphate buffer, pH 7.4. Only 0.1 mg/mL α-Synuclein, only 0.1 mg/mL Gal3, or the combination of α-Synuclein and Gal-3 were continuously stirred with a stir bar at room temperature, and samples were removed at 0, 1, 2, 3, 4, and 5 hours of incubation. Samples were then loaded onto a 4-12% Criterion™ XT Bis-Tris Protein Gel (Bio-Rad #3450124). The samples were separated at 100 V in SDS-PAGE apparatus (BioRad), and the resolved proteins were transferred onto a methanol-activated nitrocellulose membrane (Amersham GE #10600001) at 300 milliampere for 60 minutes. Nonspecific binding was blocked by incubating the membrane in 10% non-fat dried milk in TBS+Tween-20 (TBS-T) (Chem Cruz #281695) for 1 hour at room temperature. The blot was then incubated with the Syn1 mouse primary antibody (BD Biosciences #610787) specific for α-Synuclein overnight at 4° C. After three 5-minute washes in TBS-T, the membranes were incubated with anti-mouse HRP secondary antibody (Abcam #ab6789) for 1 hour at room temperature. Following three 5-minute washes in TBS-T, the membrane was incubated with chemiluminescent detection reagents (Advansta Cat #R-03021-D10 and #R-03031-D10) for 1-5 seconds. Images were captured on an Azure imager.

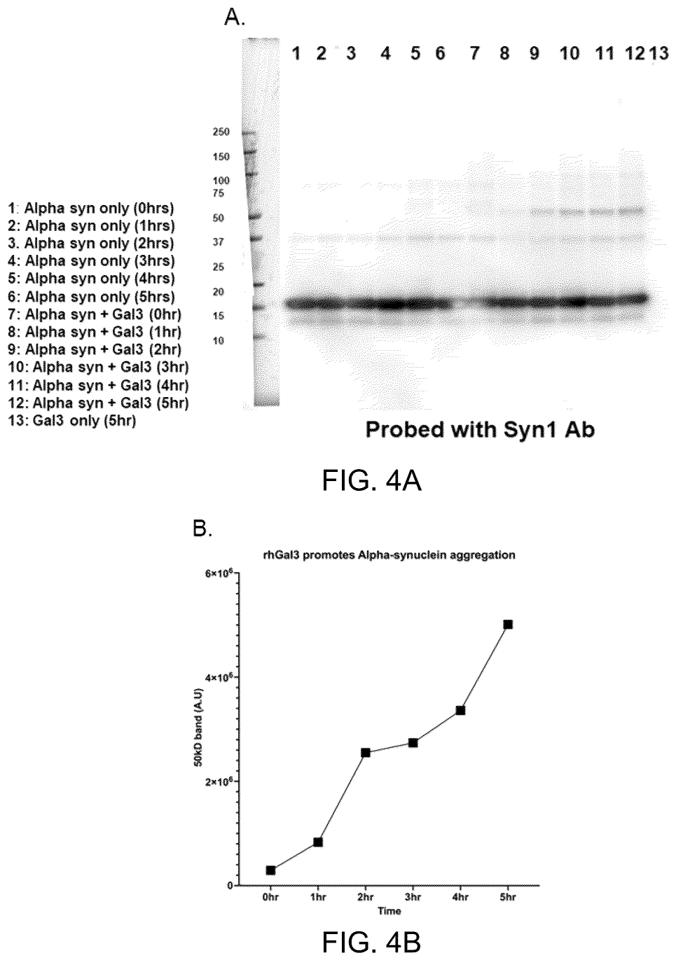

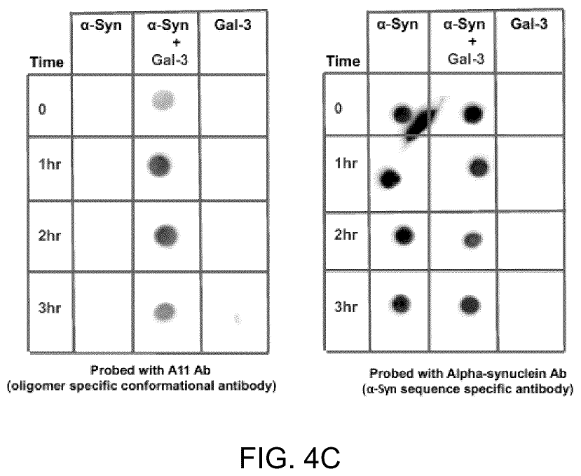

FIG. 4A-B demonstrate that Gal3 promotes the accumulation of α-Synuclein. FIG. 4A shows the Western blot where a higher molecular weight band (˜50 kDa) specifically detected by the α-Synuclein antibody was present when α-Synuclein was incubated with Gal3, but which was absent when incubated alone. FIG. 4B quantifies the progressive appearance of the aggregate over time shown in FIG. 4A.

A dot blot was performed with the samples of α-Synuclein only, α-Synuclein incubated with Gal3, and Gal3 only at time points 0, 1, 2, and 3 hours of incubation (FIG. 4C). The dot blot confirms the presence of α-Synuclein aggregates as detected by A11 antibody and α-Synuclein specific antibody.

Gal3 binding to aggregated alpha-synuclein was determined using an ELISA. Prior to the ELISA, human alpha-synuclein (R&D Systems, SP-485-500) was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 proteins (TrueBinding, QCB200349) (TrueBinding, QCB200352) (Biolegend, 599806) in separate volumes of PBS (Corning, 21-030-CM), each to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Alpha-synuclein solution for binding was prepared by diluting biotinylated aggregated alpha-synuclein in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of TMB substrate was added to each well, incubated until a sufficient signal was reached, then stopped with 25 μl of 1N HCl per well. The plate was read in a plate reader at an absorbance of 450 nm. Data was graphed using GraphPad Prism 8.0 software (GraphPad Software Inc).

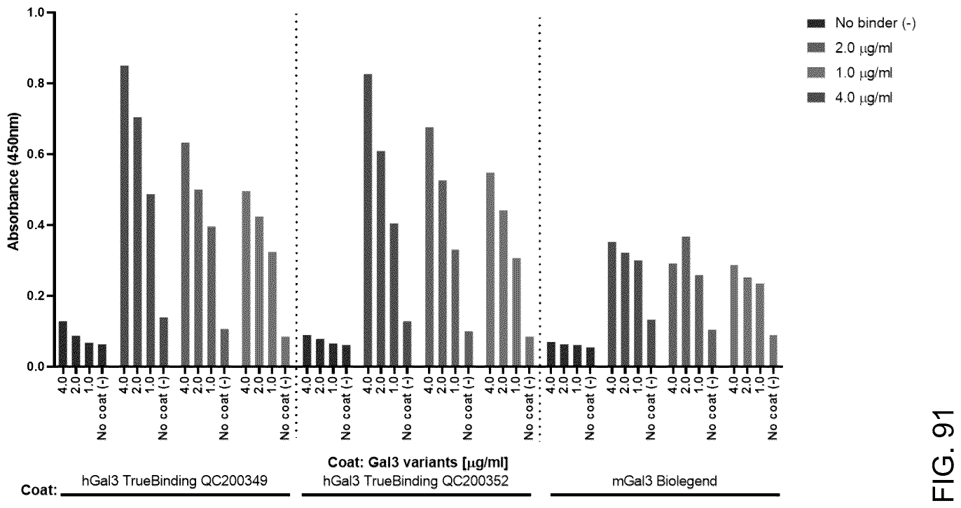

FIG. 91 depicts the results of the ELISA assay examining binding of hGal3 to aggregated alpha-synuclein. As can be seen in FIG. 91, hGal3 demonstrates strong binding to aggregated prion protein even at diminishing concentrations of aggregated alpha-synuclein or hGal3.

To identify anti-Gal3 antibodies with therapeutic potential, a screen of anti-Gal3 antibodies that block aggregation was conducted. An ELISA was used to evaluate blocking efficacies of various antibodies against galectin-3:: aggregated alpha-synuclein.

Prior to the ELISA, alpha-synuclein was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Human Galectin-3 (Gal3) protein (TrueBinding, QCB200377) was diluted in PBS to a concentration of 4 μg/ml and coated a 96-well ELISA plate by applying 35 μl to each well. After incubating the plate at 4° C. O/N, the plate was washed three times with 300 μl PBST. The plate was then blocked for an hour with 150 μl of 2% BSA in PBST at RT with gentle rocking. Thereafter, the 2% BSA in PBST was discarded and 30 μl of TB001 (TrueBinding, QC190118), TB006 (TrueBinding, QC200208), several 20H5 (TrueBinding, various QC #), or Synagis hIgG4 Isotype (TrueBinding, QC190234) (3-fold serial dilutions beginning at 10 μg/ml) in 2% BSA in PBST was added to the wells, immediately followed by the addition of 30 μl of 1 μg/ml of biotinylated alpha-synuclein in 2% BSA in PBST. The plate was incubated for an hour at RT with gentle rocking and then washed three times with 300 μl PBST. Thereafter, Avidin HRP (Biolegend, 405103) was diluted in 2% BSA in PBST (1:2000 dilution) and 25 μl was added to the wells. The plate was incubated at RT for an hour with gentle rocking and washed three times with 300 μl PBST. 50 μl of ABTS (Life Technologies, 00-2024) was added to each well to develop, then the plate was read using a plate reader at an absorbance of 405 nm. Data was graphed using GraphPad Prism 8.0 software (GraphPad Software Inc).

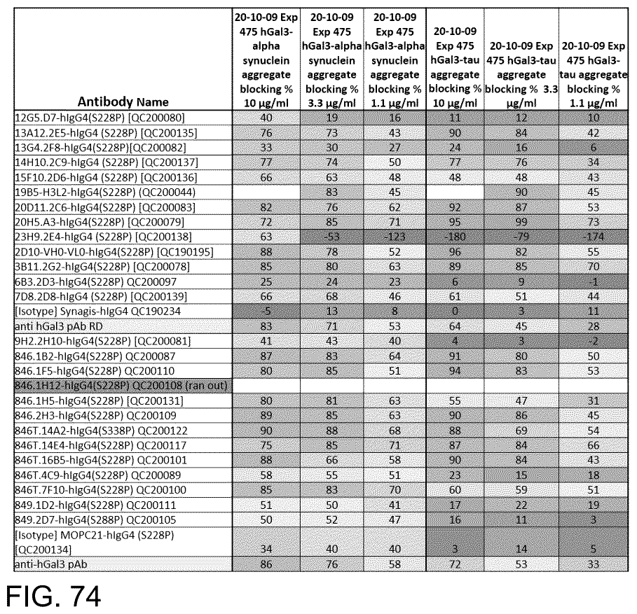

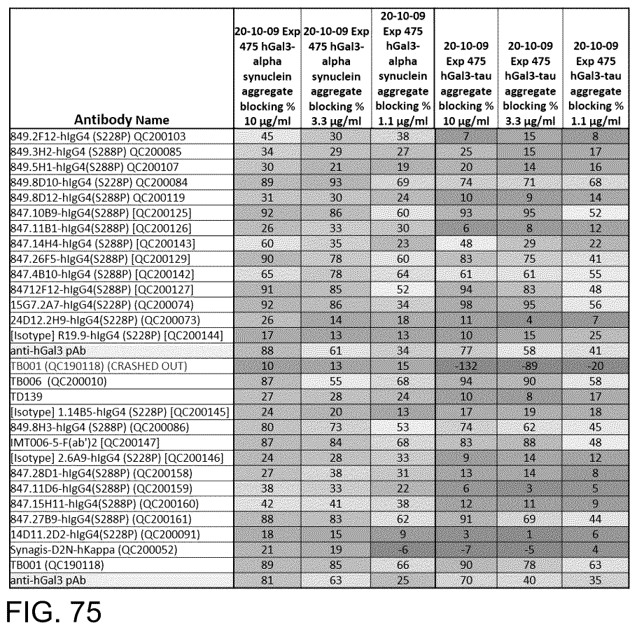

FIGS. 74 and 75 are tables depicting the quantified results of the ELISA screen.

As can be seen in FIG. 74 several anti-Gal3 blocking antibodies, including 13A12.2E5-hIgG4 (S228P) [QC200135], 14H10.2C9-hIgG4 (S228P) [QC200137], 19B5-H3L2-hIgG4(S228P) (QC200044), 20D11.2C6-hIgG4(S228P) [QC200083], 20H5.A3-hIgG4(S228P) [QC200079], 2D10-VH0-VL0-hIgG4(S228P) [QC190195], 3B11.2G2-hIgG4(S228P) [QC200078], anti hGal3 pAb RD, 846.1B2-hIgG4(S228P) QC200087, 846.1F5-hIgG4(S228P) QC200110, 846.1H5-hIgG4(S228P) [QC200131], 846.2H3-hIgG4(S228P) QC200109, 846T.14A2-hIgG4(S338P) QC200122, 846T.14E4-hIgG4(S228P) QC200117, 846T.16B5-hIgG4(S228P) QC200101, and 846T.7F10-hIgG4(S228P) QC200100, displayed significant blocking activity with these clones having a galectin-3:: aggregated alpha-synuclein blocking efficacy of greater than at least 70%.

As can be seen in FIG. 75 several anti-Gal3 blocking antibodies, including 849.8D10-hIgG4 (S228P) QC200084, 847.10B9-hIgG4(S288P) [QC200125], 847.26F5-hIgG4(S288P) [QC200129], 84712F12-hIgG4(S288P) [QC200127], 15G7.2A7-hIgG4(S228P) (QC200074), anti-hGal3 pAb, TB006 (QC200010), 849.8H3-hIgG4 (S288P) (QC200086), IMT006-5-F(ab′)2 [QC200147], 847.27B9-hIgG4(S288P) (QC200161), and TB001 (QC190118) displayed significant blocking activity with these clones having a galectin-3:: aggregated alpha-synuclein blocking efficacy of greater than at least 70%.

### Example 2. Gal3 Antibodies Block α-Synuclein Aggregate-Induced Cell Toxicity

α-Synuclein can cause neuronal cell death, leading to loss of dopaminergic neurons. To test whether anti-Gal3 antibodies could be utilized to inhibit α-Synuclein pathology, it will be first established if α-Synuclein aggregates formed in the presence of Gal3 can lead to cell toxicity. Apoptosis in the SH-SY5Y neuroblastoma cell line will be compared after treatment with α-Synuclein or α-Synuclein aggregated with Gal3.

SH-SY5Y cells will be differentiated to a dopaminergic phenotype by incubating them for four days with 10 μM retinoic acid. Differentiated cells will be re-plated and stimulated with α-Synuclein only, α-Synuclein pre-mixed with Gal3 to form aggregates, or Gal3 only. Unstimulated cells will act as controls. Apoptosis will be measured by lactate dehydrogenase (LDH) release according to manufacturer's instructions using the CytoTox 96® Non-Radioactive Cytotoxicity Assay (Promega #G1780) up to 72 hours after stimulation. It is expected that α-Synuclein pre-mixed with Gal3 to form aggregates will induce at least 10% more cell death than either unstimulated cells or α-Synuclein alone, or Gal3 alone.

To identify anti-Gal3 antibodies with therapeutic potential, a screen of anti-Gal3 antibodies that block cytotoxicity will be conducted. Various concentrations of antibodies will be incubated in the assays described above and attenuation of cell death will be assessed. Antibodies that block cytotoxicity will be identified. It is expected that antibodies that block α-Synuclein-induced cytotoxicity by at least 10% and those that do not affect aggregation will be identified. Blockers can be sub-categorized further by epitope binning.

### Example 3. Gal3 Antibodies Block α-Synuclein Aggregate-Induced Microglial Cell Activation

α-Synuclein can cause microglia activation, leading to neuroinflammation. To test whether anti-Gal3 antibodies could be utilized to inhibit α-Synuclein pathology, it will be first established if α-Synuclein aggregates formed in the presence of Gal3 lead to microglia activation. Activation in the human HMC3 (ATCC #CRL-3304) and mouse BV2 microglia cell line will be compared after treatment with α-Synuclein or α-Synuclein aggregated with Gal3.

Microglia cells will be plated and stimulated with α-Synuclein only, α-Synuclein pre-mixed with Gal3 to form aggregates, or Gal3 only. Unstimulated cells will act as controls. Activation will be measured up to 72 hours after stimulation by ELISAs against TNFα, IL-1β or IL-6, according to manufacturer's instructions. It is expected that α-Synuclein pre-mixed with Gal3 to form aggregates will induce at least 10% more TNFα, IL-1β or IL-6 than either unstimulated cells, α-Synuclein alone, or Gal3 alone.

To identify anti-Gal3 antibodies with therapeutic potential, a screen of anti-Gal3 antibodies that block activation will be conducted. Various concentrations of antibodies will be incubated in the assays described above and attenuation of activation will be assessed. It is expected that antibodies that block α-Synuclein-induced activation by at least 10% and those that do not affect activation will be identified. Blockers can be sub-categorized further by epitope binning.

### Example 4. Gal3 Antibodies Ameliorate Synucleinopathies in Small Animal Models

Parkinson's Disease, an example of a synucleinopathy, is initiated by accumulation of α-Synuclein aggregates in the substantia nigra of the brain and subsequent death of dopaminergic neurons. To identify anti-Gal3 antibodies that attenuate synucleinopathies, mouse models of Parkinson's Disease will be treated with anti-Gal3 antibodies. α-Synuclein aggregates will be injected into the brains of mice, which leads to a Parkinson's-like disease.

Animals will be tested for symptoms of Parkinson's disease with behavioral tests (Rotarod for locomotor function) and plasma dopamine level (ELISA). After symptoms are detected, animals will be treated with anti-Gal3 antibodies. At the end of the study, behavior will be tested again, biomarkers from plasma and cerebrospinal fluid (CSF) will be measured, and brain IHC will be used to detect mechanistic changes.

It is expected that anti-Gal3 antibodies improve locomotor function by at least 10%, showing improved physiology. It is expected that anti-Gal3 antibodies elevate plasma and CSF dopamine levels and the area of brain IHC staining for NeuN (a marker of neurons) by at least 10% each, showing blocks to the reduction in neurons that produce dopamine. It is expected that the area of brain section detected by A11 phospho-Ser129 α-synuclein antibody (a post-translational modification promoting oligomerization) or Congo Red (a marker of plaques) will be reduced by at least 10% upon anti-Gal3 treatment, showing blocks to α-synuclein aggregation that causes neuronal death and dopamine reduction. It is expected that the area of the brain section detected by Iba-1 (an activated microglia antibody) or GFAP (an activated astrocyte marker) will be reduced by at least 10% upon anti-Gal3 treatment, showing treatment reduces neuroinflammation.

### Example 5: Gal3 Promotes Aggregation of Tau

Neurofibrillary tangles are abnormal insoluble aggregates of Tau that cause microtubule dysfunction, neuronal degeneration, and tauopathies such as Alzheimer's disease, progressive supranuclear palsy, corticobasal degeneration and Pick's disease. To test whether anti-Gal3 antibodies could be utilized to inhibit tauopathies, it was first established that Gal3 promotes Tau aggregation. Tau was mixed with recombinant human Gal3, and molecular weight of complexes were measured with Western blot.

The 1N4RTau peptide (Tau isoform 4) (R&D Systems #SP-501-100) was diluted to a final concentration of 0.1 mg/ml by adding 10 mM sodium phosphate buffer, pH 7.4. 0.1 mg/mL Tau peptide only, 0.1 mg/mL Gal3 only, or the combination of Tau and Gal3 were continuously stirred with a stir bar for 0 and 5 hours at room temperature. Samples were then loaded onto a 4-12% Criterion™ XT Bis-Tris Protein Gel (Bio-Rad #3450124). The gel was then run at 100 V in SDS-PAGE apparatus (Biorad), and the resolved proteins were transferred onto a methanol-activated nitrocellulose membrane (Amersham GE #10600001) at 300 milliamperes for 60 minutes. Nonspecific binding was blocked by incubating the membrane in 10% non-fat dried milk in TBS+Tween-20 (TBS-T) (Chem Cruz #281695) for 1 hour at room temperature. The blot was then incubated in the Tau-5 primary antibody (Invitrogen #AHB0042) specific for total Tau protein overnight at 4° C. After three 5-minute washes in TBS-T, the membranes were incubated with anti-mouse HRP secondary antibody (Abcam #ab6789) for 1 hour at room temperature. Following three 5-minute washes in TBS-T, the membrane was incubated for 1-5 seconds in equal amounts of chemiluminescent detection reagents pre-mixed together (Advansta Cat #R-03021-D10 and #R-03031-D10). Images were captured on an Azure imager.

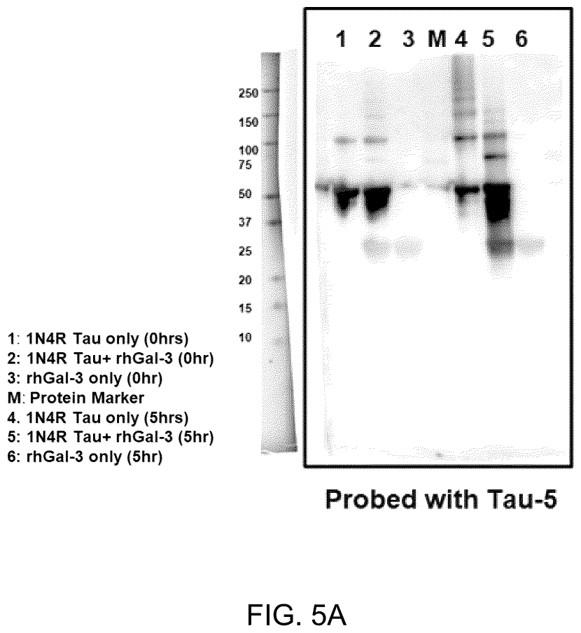

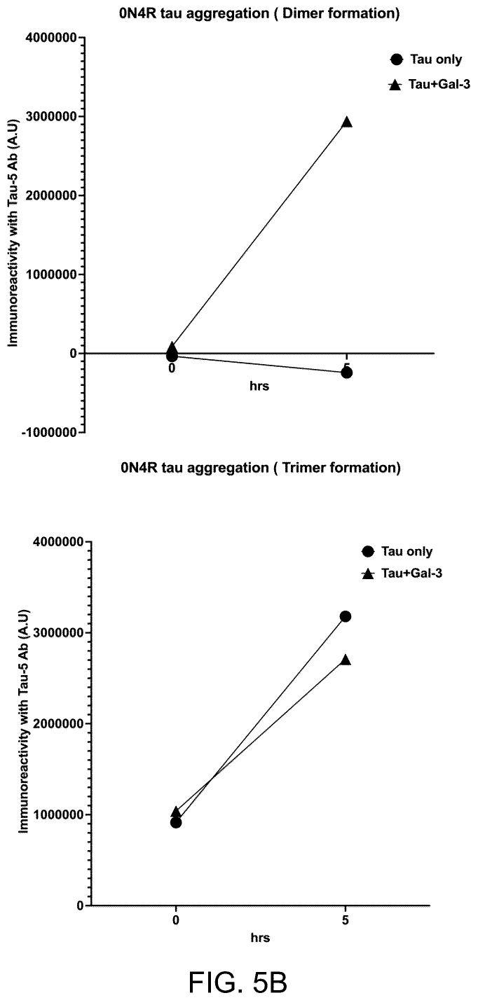

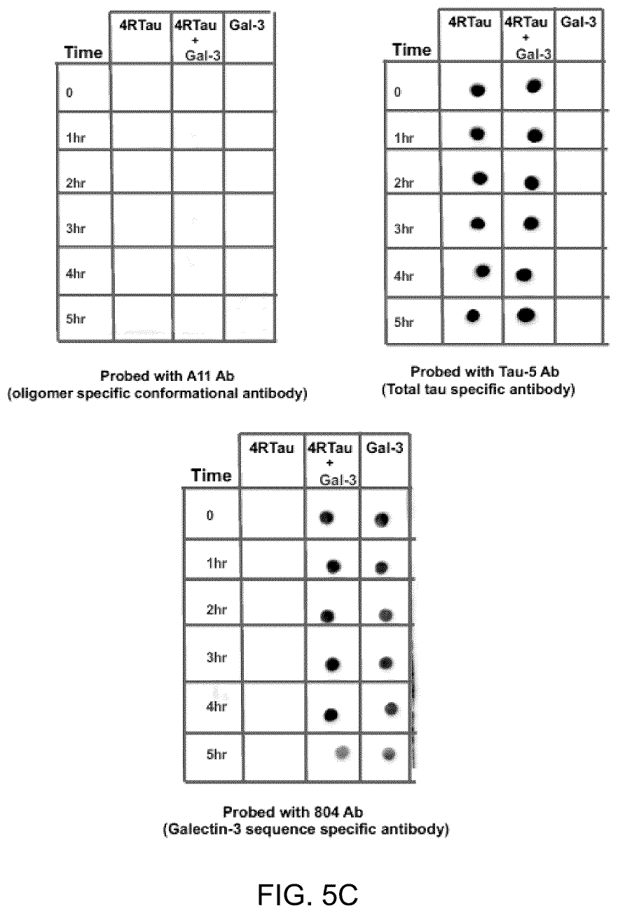

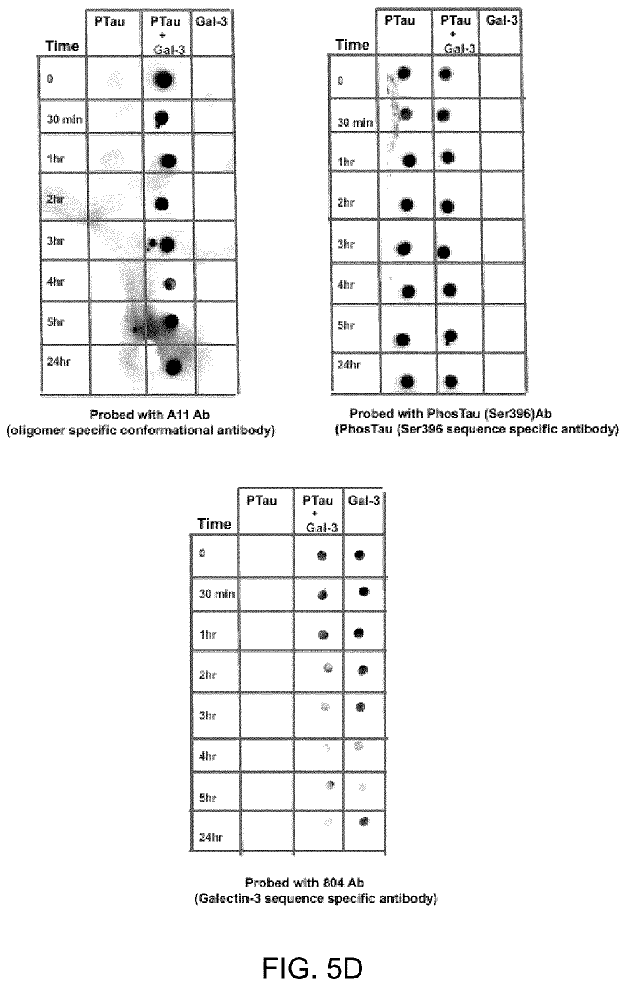

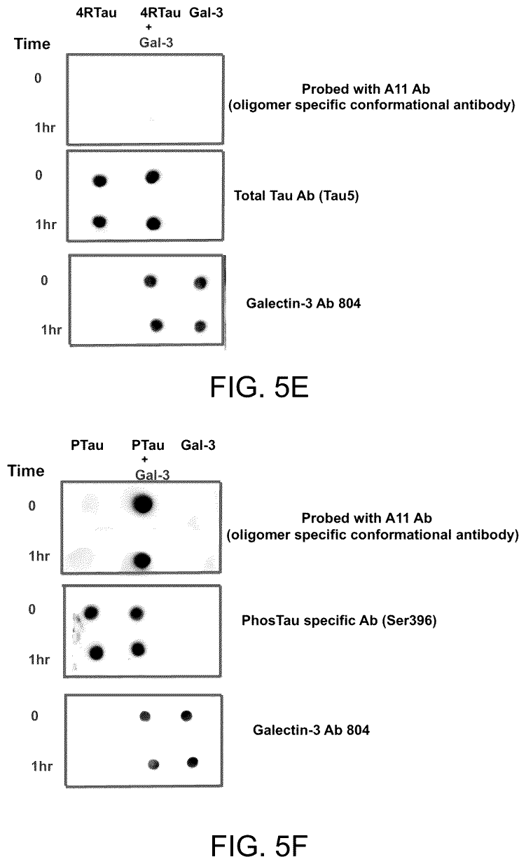

FIG. 5A demonstrates that Gal3 promoted the accumulation of higher molecular weight aggregates of Tau (˜75 and 110 kDa), which were nearly undetectable when Tau was incubated alone. The higher molecular weight aggregates observed may be due to conformational changes in the Tau proteins, in addition to additional complex and oligomer formation. No Gal3 was observed to co-migrate at the ˜75 kDa band. FIG. 5B shows the quantification of the aggregates shown in FIG. 5A.

Dot blots were performed with the samples of 1N4RTau (4RTau) only, 4RTau incubated with Gal3, and Gal3 only at time points 0, 1, 2, 3, 4, and 5 hours of incubation (FIG. 5C). The dot blots suggest that 4RTau is oligomerized very slightly by Gal3, as detected by A11 antibody and Tau-5 total tau protein specific antibody. Anti-Gal3 antibody (804) was also used to confirm the presence of Gal3 in the dot blots.

Similar dot blots were also performed following the Gal3/Tau incubation step using phosphorylated Tau (phospho-tau (S396)) after time points of 0, 0.5, 1, 2, 3, 4, 5, and 24 hours of incubation (FIG. 5D). The dot blots suggest that unlike the 4RTau samples, Gal3 heavily promoted oligomerization of phospho-tau (S396), as detected by A11 antibody and phospho-tau (S396)-specific antibody, while phospho-tau alone did not oligomerize to a significant measure in the time points tested. Anti-Gal3 antibody (804) was also used to confirm the presence of Gal3 in the dot blots.

As shown here, the promotion of aggregation of Tau (such as phospho-tau) by Gal3 is strikingly efficient. Phospho-tau oligomers can be achieved on the order of hours when incubated with Gal3 at room temperature. This is compared to previous methods, which involve the use of other inducers such as heparin or arachnoid acid, which still takes on the order of 3-4 days at 37° C.

### Example 6: Gal3 Binds Aggregated Phospho-Tau

To test whether anti-Gal3 antibodies could be utilized to inhibit tauopathies, it was first established using an ELISA that Gal3 is involved in the formation of aggregated phospho-tau.

Prior to the ELISA, human alpha-synuclein (R&D Systems, SP-485-500) was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 proteins (TrueBinding, QCB200349) (TrueBinding, QCB200352) (Biolegend, 599806) in separate volumes of PBS (Corning, 21-030-CM), each to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Phospho-tau solution for binding was prepared by diluting biotinylated aggregated phospho-tau in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of TMB substrate was added to each well, incubated until a sufficient signal was reached, then stopped with 25 μl of 1N HCl per well. The plate was read in a plate reader at an absorbance of 450 nm. Data was graphed using GraphPad Prism 8.0 software (GraphPad Software Inc).

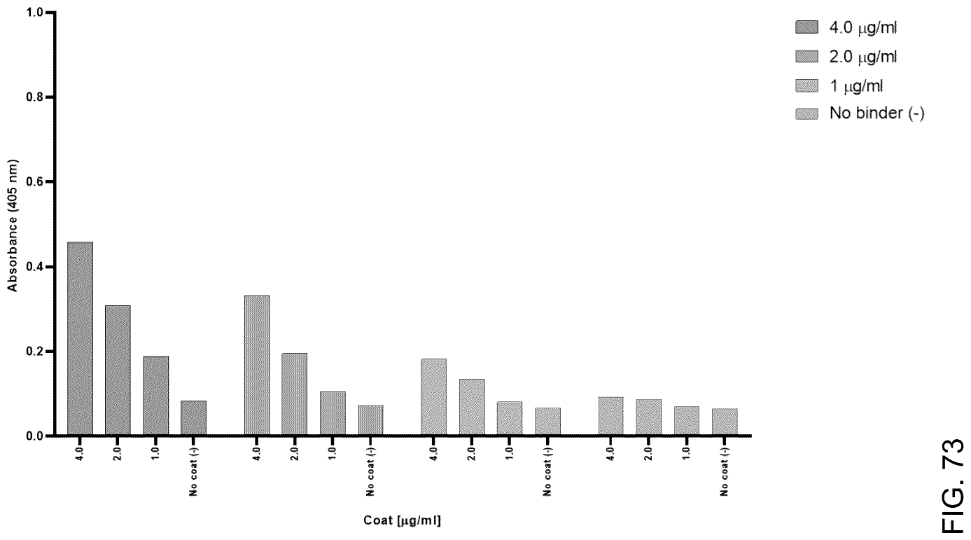

FIG. 73 depicts the results of the ELISA assay examining binding of hGal3 to aggregated phospho-Tau. As can be seen in FIG. 73, hGal3 demonstrates weak binding to aggregated phospho-Tau.

### Example 7: Gal3 Antibodies Block Gal3-Induced Aggregation of Phospho-Tau

To identify anti-Gal3 antibodies with therapeutic potential, a screen of anti-Gal3 antibodies that block aggregation was conducted. An ELISA was used to evaluate blocking efficacies of various antibodies against galectin-3:: aggregated phospho-tau.

Prior to the ELISA, phospho-tau was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Human Galectin-3 (Gal3) protein (TrueBinding, QCB200377) was diluted in PBS to a concentration of 4 μg/ml and coated a 96-well ELISA plate by applying 35 μl to each well. After incubating the plate at 4° C. O/N, the plate was washed three times with 300 μl PBST. The plate was then blocked for an hour with 150 μl of 2% BSA in PBST at RT with gentle rocking. Thereafter, the 2% BSA in PBST was discarded and 30 μl of TB001 (TrueBinding, QC190118), TB006 (TrueBinding, QC200208), several 20H5 (TrueBinding, various QC #), or Synagis hIgG4 Isotype (TrueBinding, QC190234) (3-fold serial dilutions beginning at 10 μg/ml) in 2% BSA in PBST was added to the wells, immediately followed by the addition of 30 μl of 1 μg/ml of biotinylated phospho-tau in 2% BSA in PBST. The plate was incubated for an hour at RT with gentle rocking and then washed three times with 300 μl PBST. Thereafter, Avidin HRP (Biolegend, 405103) was diluted in 2% BSA in PBST (1:2000 dilution) and 25 μl was added to the wells. The plate was incubated at RT for an hour with gentle rocking and washed three times with 300 μl PBST. 50 μl of ABTS (Life Technologies, 00-2024) was added to each well to develop, then the plate was read using a plate reader at an absorbance of 405 nm. Data was graphed using GraphPad Prism 8.0 software (GraphPad Software Inc).

FIGS. 74 and 75 are tables depicting the quantified results of the ELISA screen.

As can be seen in FIG. 74 several anti-Gal3 blocking antibodies, including 13A12.2E5-hIgG4 (S228P) [QC200135], 14H10.2C9-hIgG4 (S228P) [QC200137], 19B5-H3L2-hIgG4(S228P) (QC200044), 20D11.2C6-hIgG4(S228P) [QC200083], 20H5.A3-hIgG4(S228P) [QC200079], 2D10-VH0-VL0-hIgG4(S228P) [QC190195], 3B11.2G2-hIgG4(S228P) [QC200078], 846.1B2-hIgG4(S228P) QC200087, 846.1F5-hIgG4(S228P) QC200110, 846.2H3-hIgG4(S228P) QC200109, 846T.14A2-hIgG4(S338P) QC200122, 846T.14E4-hIgG4(S228P) QC200117, and 846T.16B5-hIgG4(S228P) QC200101, displayed significant blocking activity with these clones having a galectin-3:: aggregated phospho-tau blocking efficacy of greater than at least 70%.

As can be seen in FIG. 75 several anti-Gal3 blocking antibodies, including 847.26F5-hIgG4(S288P) [QC200129], 84712F12-hIgG4(S288P) [QC200127], 15G7.2A7-hIgG4(S228P) (QC200074), TB006 (QC200010), 849.8H3-hIgG4 (S288P) (QC200086), IMT006-5-F(ab′)2 [QC200147], 847.27B9-hIgG4(S288P) (QC200161), and TB001 (QC190118), displayed significant blocking activity with these clones having a galectin-3 aggregated phospho-tau blocking efficacy of greater than at least 70%.

### Example 8: Gal3 Antibodies Block Tau Aggregate-Induced Neuronal Cell Toxicity

Tau can cause neuronal cell death, leading to loss of neurons. To test whether anti-Gal3 antibodies could be utilized to inhibit Tau pathology, it will be first established if Tau aggregates formed in the presence of Gal3 lead to cell toxicity. Apoptosis in the SH-SY5Y neuroblastoma cell line will be compared after treatment with Tau pre-incubated with or without Gal3.

SH-SY5Y cells will be plated and stimulated with Tau only, Tau pre-mixed with Gal3 to form aggregates, or Gal3 only. Unstimulated cells will act as controls. Cell death will be measured by lactate dehydrogenase (LDH) release according to manufacturer's instruction using the CytoTox 96® Non-Radioactive Cytotoxicity Assay (Promega #G1780) up to 72 hours after stimulation. It is expected that Tau pre-mixed with Gal3 to form aggregates will induce at least 10% more cell death than either unstimulated cells, α-Synuclein alone, or Gal3 alone.

To identify anti-Gal3 antibodies with therapeutic potential, a screen of anti-Gal3 antibodies that block cytotoxicity will be conducted. Various concentrations of antibodies will be incubated in the assays described above and attenuation of cell death will be assessed. Antibodies that block cytotoxicity by at least 10% and those that do not affect cytotoxicity will be identified. Blockers can be sub-categorized further by epitope binning.

### Example 9: Gal3 Antibodies Block Tau Aggregate-Induced Microglial Cell Activation

Tau aggregates can cause microglia activation, leading to neuroinflammation. To test whether anti-Gal3 antibodies could be utilized to inhibit Tau pathology, it will be first established if Tau aggregates formed in the presence of Gal3 lead to microglia activation. Activation in the human HMC3 (ATCC #CRL-3304) and mouse BV2 microglia cell line will be compared after treatment with Tau or Tau aggregated with Gal3.

Microglia cells will be plated and stimulated with Tau only, Tau pre-mixed with Gal3 to form aggregates, or Gal3 only. Unstimulated cells will act as controls. Activation will be measured up to 72 hours after stimulation by ELISAs against TNFα, IL-1β or IL-6, according to manufacturer's instructions. It is expected that Tau pre-mixed with Gal3 to form aggregates will induce at least 10% more TNFα, IL-1β or IL-6 than either unstimulated cells or Tau or Gal3 alone.

To identify anti-Gal3 antibodies with therapeutic potential, a screen of anti-Gal3 antibodies that block activation will be conducted. Various concentrations of antibodies will be incubated in the assays described above and attenuation of activation is assessed. It is expected that antibodies that block Tau-induced activation by at least 10% and those that do not affect activation will be identified. Blockers can be sub-categorized further by epitope binning.

### Example 10: Gal3 Antibodies Ameliorate Tauopathies in Small Animal Models

Alzheimer's disease, an example of a tauopathy, can be initiated by Tau aggregates. To identify anti-Gal3 antibodies that attenuate diseases driven by Tau aggregation, mouse models of tauopathies will be treated with anti-Gal3 antibodies. Tau aggregate injection into the brains of C57BL/6J mice leads to seeding of endogenous Tau aggregation and symptoms of dementia. In a second model, Aβ42 aggregate injection into the brains of C57BL/6J mice leads to Tau hyper-phosphorylation and aggregation and symptoms of dementia.

After Tau or Aβ42 aggregate injection, animals will be tested for symptoms of dementia with behavioral tests (Rotarod for locomotor function; Morris water maze or Y maze for cognitive function). After symptoms are detected, animals will be treated with anti-Gal3 antibodies. At the end of the study, behavior will be tested again, biomarkers from plasma and cerebrospinal fluid (CSF) will be measured and brain IHC will be used to detect mechanistic changes.

It is expected that anti-Gal3 antibodies improve locomotor or cognitive function by at least 10%, showing improved physiology. It is expected that anti-Gal3 antibodies will reduce plasma and CSF biomarkers (e.g. Aβ42, total Tau, phospho-Tau, neurofilament-light, neurofilament-heavy or Gal3) by at least 10%, showing improved pathology or target engagement using biomarkers. It is expected that the area of brain IHC staining for NeuN (a marker of neurons) will be increased by at least 10% each, showing blocks to the reduction in neurons. It is expected that the area of brain section detected by A11AT8 (a hyper-phosphorylated Tau antibody), AT100 (an aggregated Tau protein) or Congo Red (a marker of fibrils) will be reduced by at least 10% upon anti-Gal3 treatment, showing blocks to Tau aggregation that causes neuronal death. It is expected that the area of the brain section detected by Iba-1 (an activated microglia antibody) or GFAP (an activated astrocyte marker) will be reduced by at least 10% upon anti-Gal3 treatment, showing treatment reduces neuroinflammation. It is expected that the area of the brain section detected by Prussian Blue) will be reduced by at least 10% upon anti-Gal3 treatment, showing treatment reduces microhemorrhage.

### Example 11. Gal3 Antibodies Block Gal3-Induced Aggregation of TDP-43

TDP-43 proteinopathies can cause neurodegeneration. For instance, amyotrophic lateral sclerosis (ALS) and Frontotemporal lobar degeneration (FTLD) are caused by the neuronal death induced by mis-localized and/or aggregated TDP-43. To test whether anti-Gal3 antibodies could be utilized to inhibit TDP-43 proteinopathies, it was first established that Gal3 promotes TDP-43 aggregation. TDP-43 peptide was mixed with recombinant human Gal3 and aggregation was measured with dot blots and Western blot.

TDP-43 and Gal3 or their combination in 10 mM Phosphate buffer pH 7.4 were mixed continuously with a stir bar at 37° C. and aliquots were removed at 0, 0.5, 1, 2, 3, 4, 5 and 24 hours, and then frozen. To detect aggregates, samples were rapidly thawed at 37° C. and then 2 μL were loaded onto a nitrocellulose membrane and air-dried for 1 hour at room temperature. Equal loading was confirmed with Ponceau Red staining. After the Ponceau Red was washed off, the nitrocellulose was blocked in 10% non-fat milk/TBS-Tween-20, and then incubated for 1 hour with A11, anti-TDP-43, or anti-Gal3 antibodies. After washing, the nitrocellulose was incubated for 1 hour with 1:5000 Donkey anti-rabbit HRP detection antibody (Jackson ImmunoResearch #711-035-152) or donkey anti-mouse HRP (Jackson ImmunoResearch #715-035-151) secondary antibody. After a final wash, bands were detected with a chemiluminescent substrate (1:1 mixture of Advansta Cat #R-03021-D10 and #R-03031-D10). Images were captured on an Azure imager.

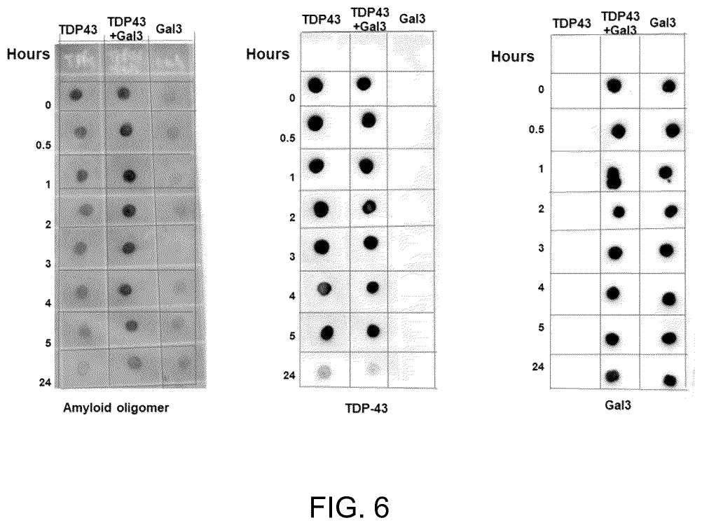

FIG. 6 shows a dot blot following TDP-43 aggregation. The time points when the samples were collected for the analysis are indicated. For all of the time points tested, the intensity of the amyloid oligomer-specific dots was stronger when Gal3 was added to the mixture, demonstrating that Gal3 promotes aggregation of TDP-43. Equal loading was confirmed by the intensity of blots probed with anti-TDP-43 antibody. Equal amounts of Gal3 were confirmed by the intensity of blots probed with anti-Gal3 antibody

TDP-43 was diluted to a final concentration of 0.1 mg/ml by adding 10 mM sodium phosphate buffer, pH 7.4. Only 0.1 mg/mL TDP-43, only 0.1 mg/mL Gal3, or the combination of TDP-43 and Gal3 were continuously stirred with a stir bar at room temperature, and samples were removed at 0, 1, 2, 3, 4, and 5 hours of incubation. Samples were then loaded onto a 4-12% Criterion™ XT Bis-Tris Protein Gel (Bio-Rad #3450124). The samples were separated at 100 V in SDS-PAGE apparatus (Biorad), and the resolved proteins were transferred onto a methanol-activated nitrocellulose membrane (Amersham GE #10600001) at 300 milliamperes for 60 minutes. Nonspecific binding was blocked by incubating the membrane in 10% non-fat dried milk in TBS+Tween-20 (TBS-T) (Chem Cruz #281695) for 1 hour at room temperature. The blot was then incubated with a TDP-43 primary antibody overnight at 4° C. After three 5-minute washes in TBS-T, the membranes were incubated with anti-mouse HRP secondary antibody (Abcam #ab6789) for 1 hour at room temperature. Following three 5-minute washes in TBS-T, the membrane was incubating in a mixing equal amounts of chemiluminescent detection reagents (Advansta Cat #R-03021-D10 and #R-03031-D10) for 1-5 seconds. Images were captured on an Azure imager and quantified. Blots were then stripped, blocked with milk, and re-probed with anti-Gal3 antibody. After washing, Gal3 was detected using an anti-mouse HRP secondary antibody (Abcam #ab6789) for 1 hour at room temperature. Following three 5-minute washes in TBS-T, the membrane was incubated in equal amounts of chemiluminescent detection reagents pre-mixed together (Advansta Cat #R-03021-D10 and #R-03031-D10) for 1-5 seconds. Images were captured on an Azure imager.

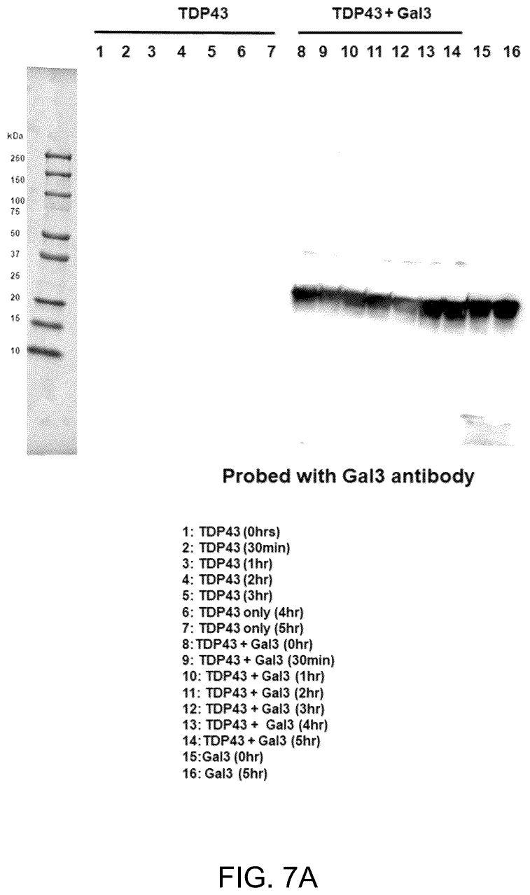

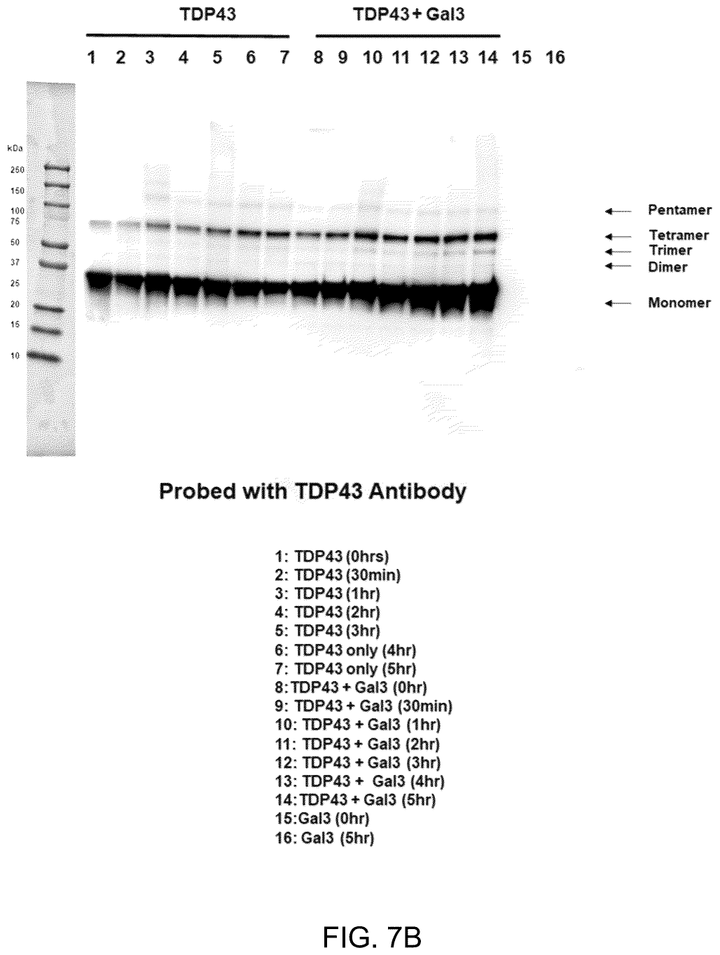

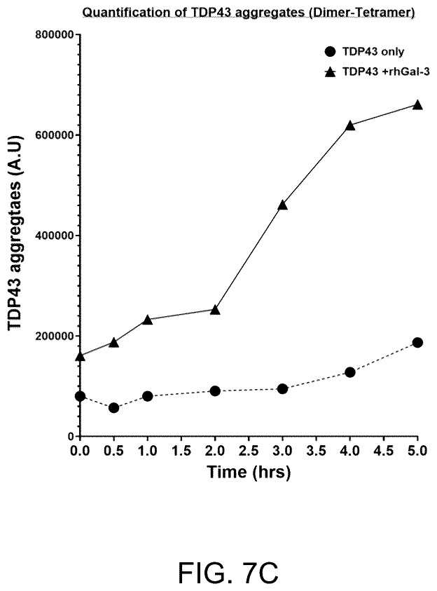

FIG. 7A shows a Western blot (re-probed after probing with anti-TDP-43 as shown in FIG. 7B) where Gal3 is detected at similar intensity in all samples that contained Gal3 during incubation. FIG. 7B shows a Western blot where higher molecular weight bands specifically detected by the TDP-43 antibody are present when TDP-43 was incubated with Gal3, but which was absent when incubated alone. FIG. 7C quantifies the progressive appearance of dimers and tetramers over time in the Western blot shown in FIG. 7B. This confirms the results of A11-probed dot blots demonstrating the appearance of amyloids when Gal3 was mixed with TDP-43.

To identify anti-Gal3 antibodies with therapeutic potential, a screen of anti-Gal3 antibodies that block aggregation will be conducted. Various concentrations of antibodies will be incubated with TDP-43 with or without Gal3, and A11 will be used to detect oligomers on dot blots. It is expected that antibodies that block TDP-43 aggregation by at least 10% and those that do not affect it will be identified. Blockers can be sub-categorized further by epitope binning.

### Example 12. Gal3 Antibodies Block TDP-43 Aggregation-Induced Neuronal Cytotoxicity and Microglial Cell Activation

TDP-43 aggregates can cause neuronal death autonomously or non-autonomously via activation of microglia. To identify Gal3 antibodies that reduce neuronal death induced by TDP-43 aggregates, it will first be established that Gal3 promotes TDP-43-induced cell death. Viability in neuronal cell lines in the presence or absence of microglial cell lines (mouse BV2 source; human HMC3 ATCC #CRL-3304) will be compared after treatment with TDP-43 or TDP-43 aggregated with Gal3.

Neuronal cells alone, microglial cells alone or the co-culture of microglial and neuronal cell will be grown in a 96 well plate, and then culture medium will be replaced with serum-free DMEM. TDP-43 alone, TDP-43 pre-mixed with Gal3 to form aggregates, or Gal3 alone will be added in concentrations from 2-25 μL to the plated cells. After 8-96 hours of incubation, supernatants will be collected to measure microglial activation and neuronal cells viability as described below.

Activation of microglia by TDP-43 aggregates will be measured with ELISA kits for the pro-inflammatory mediators IL-6, TNFα and IL-1β. It is expected that at least one mediator will be increased by at least 10% when stimulated by TDP-43 mixed with Gal3 compared to TDP-43 or Gal3 mixed alone. This demonstrates that Gal3-induced TDP-43 aggregates activate microglia.

For neuronal cell viability, cells will be stained against CD68, NeuN and Annexin V. Apoptosis will be assessed using Annexin V. Samples will be run on an Attune flow cytometer (Thermo-Fisher) and analyzed on Flow Jo software (TreeStar). Samples will be gated into CD68+ microglia and NeuN+ neurons and then the percent of Annexin V positive cells will be quantified within each gate. It is expected that cells incubated with the combination of Gal3 and TDP-43 will be at least 10% less viable than cells incubated with TDP-43 or Gal3 alone. It is expected that this occurs either in the presence or absence of microglia in the co-culture. These data demonstrate that Gal3-induced TDP-43 aggregates reduce neuronal viability in a cell autonomous or cell non-autonomous manner.

To identify anti-Gal3 antibodies with therapeutic potential, a screen of anti-Gal3 antibodies that block cell activation or cytotoxicity will be conducted. Various concentrations of antibodies will be incubated in the assays described above and attenuation of activation or cell death will be assessed. Antibodies that block activation or cytotoxicity by at least 10% will be identified. Blockers can be sub-categorized further by epitope binning.

### Example 13. Gal3 Antibodies Ameliorate ALS

ALS, an example of a TDP-43 proteinopathy, is caused by accumulation of toxic TDP-43 aggregates. To identify anti-Gal3 antibodies that can ameliorate ALS caused by TDP-43 aggregates, we will use a model of ALS. ALS is induced by bi-lateral stereotactic injection of TDP-43 oligomerized in the presence of Gal3. Un-injected animals will serve as healthy controls. To follow ALS progression, locomotor function will be assessed using the Rotarod test and cognitive function will be assessed using Morris water maze and Y maze tests. Decreased locomotor or cognitive function compared to healthy controls indicates ALS-like disease. After ALS symptoms are noted in the TDP-43-injected animals, anti-Gal3 antibodies or isotype controls will be dosed. Healthy controls will remain untreated. Treatment-induced changes to locomotor and cognitive function will again be assessed before sacrifice up to 8 weeks later. Pathophysiological changes caused by Gal3 antibody treatment will be assessed with IHC and ELISA on the brain and ELISA on plasma and CSF

It is expected that locomotor function will be improved by at least 10% in Gal3-treated animals compared to isotype control-treated animals. It is expected that cognitive function will be improved by at least 10% in one or more tests in Gal3-treated animals compared to isotype control-treated animals. It is expected that the area stained by Iba-1, MHC-II, GFAP or other inflammatory markers will be reduced by at least 10% in Gal3-treated animals compared to isotype control-treated animals in IHC. It is expected that the area stained by MAP-2, NeuN or other neuronal markers will be increased by at least 10% in anti-Gal3-treated animals compared to isotype control-treated animals in IHC. It is expected that the area stained by TDP-43, Aβ42, total tau, phosphorylated tau or other neurodegeneration markers will be reduced by at least 10% in anti-Gal3-treated animals compared to isotype control-treated animals in IHC. It is expected that the level of IL-6, IL-1β, TNFα or other inflammatory markers will be reduced by at least 10% in anti-Gal3-treated animals compared to isotype control-treated animals in ELISA of brain lysates. It is expected that the level of TDP-43, Aβ42, total tau, phosphorylated tau, NF-L, NF-M, NF-H, S100β, Cystatin C, PGRN, GFAP, BDNF, MCP-1, YKL-40, CHIT1, Gal3 or other ALS biomarker will be reduced by at least 10% in anti-Gal3-treated animals compared to isotype control-treated animals in ELISA of plasma or CSF.

### Example 14: Gal3 Antibodies Block Gal3-Induced Aggregation of Transthyretin

Cardiac amyloidosis (primary, secondary, familial or senile) is caused by the accumulation of amyloid aggregates or misfolding of amyloid proteins, such as desmin light chain or Transthyretin (TTR). TTR is a transport protein that carries thyroxine and retinol (vitamin A)-retinol binding protein complex. TTR amyloidosis (ATTR) can occur when TTR forms fibrils which are deposited in the myocardium, causing cardiomyopathy.

To test whether anti-Gal3 antibodies could be utilized to inhibit cardiac amyloidosis, it was established that Gal3 promotes TTR aggregation. TTR was mixed with recombinant human Gal3 and aggregation was measured. 0.1 mg/mL of transthyretin recombinant human protein (Invitrogen #LFP0054) was mixed with continuous stirring at room temperature with or without 0.1 mg/mL of Gal3 added. At 0 hours (immediately after mixing together) and at multiple time points up to 6 days later, 5 μL aliquots were removed and immediately frozen at −80° C. After all samples were collected, they were analyzed by Western and/or dot blots.

To detect enhanced protein size seen in aggregates, samples were rapidly thawed at 37° C. and then 5 μL was loaded onto a 4-12% Criterion™ XT Bis-Tris Protein Gel (Bio-Rad #3450124), transferred to nitrocellulose (Amersham GE #10600001), and equal loading was confirmed with Ponceau Red staining. After the Ponceau Red was washed off, the nitrocellulose was blocked in 10% non-fat milk/TBS-Tween-20, then incubated with 1:2000 of TTR antibody (Invitrogen #PA527220). After washing, the nitrocellulose was incubated for 1 hour with 1:5000 donkey anti-rabbit HRP detection antibody (Jackson ImmunoResearch #711-035-152). After a final wash, bands were detected with a chemiluminescent substrate (1:1 mixture of Advansta Cat #R-03021-D10 and #R-03031-D10). Images were captured on an Azure imager. A shift in the molecular weight of transthyretin indicated oligomerization.

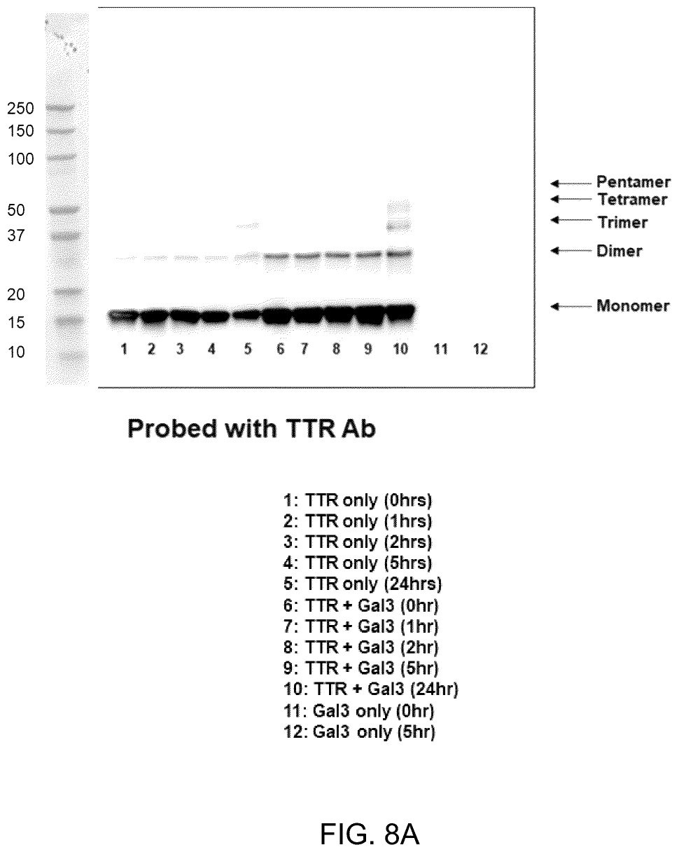

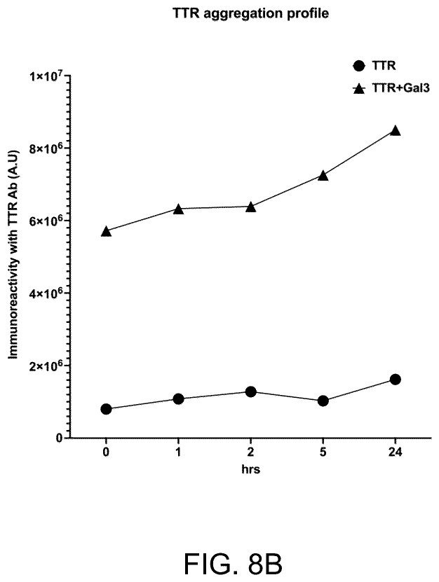

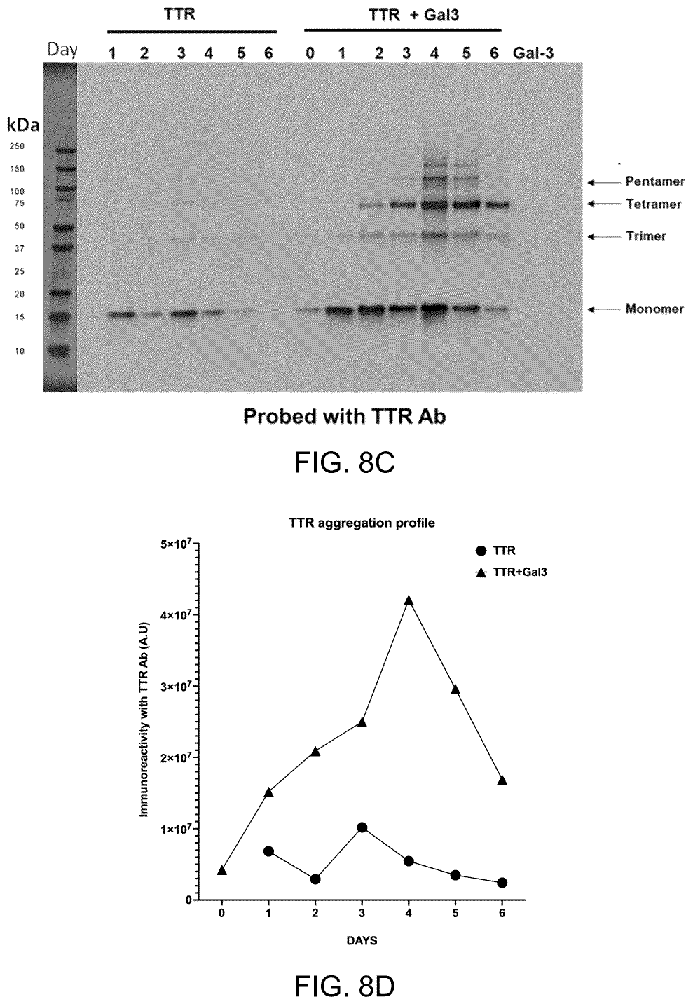

FIG. 8A shows that Gal3 enhanced the level of dimeric TTR early (0-5 hours) after co-incubation and FIG. 8B quantifies the appearance of the dimers. Protein concentration was established prior to loading of the gels, and observed difference in signal is suspected to be due to partial epitope masking. After 24 hours, Gal3 also enhanced the level of trimers, tetramers and pentamers. FIG. 8C showed that Gal3 enhanced aggregation of TTR until at least 6 days after mixing and FIG. 8D quantifies the aggregates of FIG. 8C.

To identify anti-Gal3 antibodies with therapeutic potential, a screen of anti-Gal3 antibodies that block aggregation will be conducted. Various concentrations of antibodies will be incubated with Gal3 and TTR, and Western blots, as described above, will be used to detect antibodies that block aggregation. It is expected that antibodies that block TTR aggregation by at least 10% and those that do not affect aggregation will be identified. Blockers can be sub-categorized further by epitope binning.

### Example 15: Gal3 Antibodies Block Transthyretin Aggregate-Induced Cardiac Cell Toxicity

TTR aggregates can cause cardiomyocyte apoptosis. To identify Gal3 antibodies that reduce cardiomyocyte death induced by TTR aggregates, it will be first established that Gal3 promotes TTR-induced cell death. Viability in H9C2 Rat cardiomyocyte (ATCC-CRL-1446), AC16 Human Cardiomyocyte (Sigma-SCC109) or HL-1 Cardiac Muscle (Sigma-SCC065) cell lines will be compared after treatment with TTR pre-incubated with or without Gal3.

5×10³ cells will be grown overnight in a 96 well plate, and then medium will be replaced with serum-free DMEM. Cells will be stimulated with 2-25 μL of TTR alone, TTR pre-mixed with Gal3 to form aggregates, or Gal3 alone. After 8-48 hours of incubation, the cell viability will be measured with Cell Titer Blue (Promega #G8080), according to manufacturer's instructions. It is expected that cells incubated with the combination of Gal3 and TTR will be at least 10% less viable than cells incubated with TTR or Gal3 alone.

To identify anti-Gal3 antibodies with therapeutic potential, a screen of anti-Gal3 antibodies that block cytotoxicity will be conducted. Various concentrations of antibodies will be incubated in the assays described above and attenuation of cell death will be assessed. Antibodies that block cytotoxicity by at least 10% will be identified. Blockers can be sub-categorized further by epitope binning.

### Example 16: Gal3 Antibodies Ameliorate Cardiac Amyloidosis

To identify anti-Gal3 antibodies that can ameliorate cardiac amyloidosis, two models of cardiomyopathy will be used.

TTR amyloid deposition in the myocardium results in arrhythmia (atrial fibrillation) and/or heart failure. TTR aggregates generated in vitro in the presence of Gal3 will be injected into the apex of the heart in C57BL6/J mice or Sprague-Dawley rats. Anti-Gal3 antibodies will be injected intra-peritoneally. TTR aggregates and fibrils will be detected on heart sections with A11 amyloid oligomer IHC and Congo Red. TTR aggregation will be confirmed by detecting shifts in TTR molecular weight from heart lysates probed with a TTR antibody on Western blots. Aggregate deposition in the heart will be quantified with Technetium pyrophosphate (99mTc-PYP) single photon emission computed tomography (SPECT). Cardiac injury will be measured by plasma biomarkers. Cardiac function will be measured using echocardiography.

It is expected that there will be at least a 10% reduction in the levels of aggregates in heart sections and lysates when animals are treated with anti-Gal3 antibodies compared to untreated animals. It is expected that there will be at least a 10% reduction in the oligomer deposition in the heart using SPECT when animals are treated with anti-Gal3 antibodies compared to untreated animals. It is expected that there will be at least a 10% change to ejection fraction on echocardiography when animals are treated with anti-Gal3 antibodies compared to untreated animals. It is expected that there will be at least a 10% reduction in biomarker levels (e.g. Brain natriuretic peptide, Soluble suppression of tumorigenesis-2, TTR, Gal3) when animals are treated with anti-Gal3 antibodies compared to untreated controls.

Transverse aortic constriction (TAC) will be performed on C57BL/6J mice to induce cardiac hypertrophy. Animals will be treated with anti-Gal3 antibodies. The presence of amyloid aggregates in the heart will be quantified by staining heart sections with A11 and Congo Red. Fibrosis will be measured on heart sections with Masson trichome and Picrosirius Red. Cardiac injury will be measured by quantifying levels of plasma biomarkers (ssT2, BNP, TTR, desmin) by ELISA. Cardiac function will be determined using echocardiograms (left ventricular wall thickness and ejection fraction).

It is expected that Gal3 antibodies will reduce aggregates and fibrils in the heart by at least 10% compared to untreated animals. It is expected that Gal3 antibodies will reduce fibrosis in the heart by at least 10% compared to untreated animals. It is expected that Gal3 antibodies will reduce cardiac injury by at least 10% compared to untreated animals. It is expected that Gal3 antibodies will improve cardiac function by at least 10% compared to untreated animals.

### Example 17: Gal3 Antibodies Block Gal3-Induced Aggregation of Uromodulin

Chronic kidney disease can be caused by the aggregation of amyloids such as light chain or uromodulin. Uromodulin is the most abundant protein in urine. Under some conditions, it can aggregate and coalesce into a gel-like substance known as renal casts that can obstruct fluid flow. To test whether anti-Gal3 antibodies could be utilized to inhibit uromodulin-mediated amyloidosis, it will be first established that Gal3 promotes uromodulin aggregation. Uromodulin will be mixed with recombinant human Gal3 and oligomerization will be measured with dot blots.

Uromodulin (15 μg) (Biovendor #RD172163100), Gal3 (15 μg) or their combination will be mixed continuously with a stir bar at 37° C. and aliquots removed at 0, 1, 2, 3 and 24 hours, and then frozen. To detect aggregates, samples will be rapidly thawed at 37° C. and then 2 μL will be loaded onto a nitrocellulose membrane and air-dried for 1 hour at room temperature. Equal loading will be confirmed with Ponceau Red staining. After the Ponceau Red is washed off, the nitrocellulose will be blocked in 10% non-fat milk/TBS-Tween-20, and then incubated for 1 hour with A11 antibody. After washing, the nitrocellulose will be incubated for 1 hour with 1:5000 Donkey anti-rabbit HRP detection antibody (Jackson ImmunoResearch #711-035-152). After a final wash, bands will be detected with a chemiluminescent substrate (1:1 mixture of Advansta Cat #R-03021-D10 and #R-03031-D10). Images will be captured on an Azure imager. It is expected that at 1 and 2 hours, the intensity of the amyloid oligomer-specific dots will be stronger when Gal3 was co-incubated, demonstrating that Gal3 promotes aggregation of uromodulin.

To identify anti-Gal3 antibodies with therapeutic potential, a screen of anti-Gal3 antibodies that block aggregation will be conducted. Various concentrations of antibodies will be incubated with uromodulin with or without Gal3, and A11 will be used to detect oligomers on dot blots. It is expected that antibodies that fully or partially block uromodulin aggregation by at least 10% and those that do not affect it will be identified. Blockers can be sub-categorized further by epitope binning.

### Example 18: Gal3 Antibodies Ameliorate Uromodulin-Associated Kidney Disease

Uromodulin synthesized by kidney tubular cells assembles into filaments that form a mesh-like structure where it traps bacteria and light chain immunoglobulins with its gel like structure. In a disease state, uromodulin filaments bind with sodium and form hard structures called casts, which cause decline in kidney function. In SHR rats, uromodulin amyloids develop with aging as the casts are formed.

To test if anti-Gal3 antibodies can ameliorate kidney disease caused by amyloids, aged SHR rats will be treated with Gal3 antibodies or isotype controls. Overall health will be measured by body, heart and kidney weight. Kidney dysfunction will be measured by the volume of urine produced while in metabolic cages. The number and size of casts in animals will be quantified using H&E staining on kidney sections. Kidney injury will be detected with biomarkers from plasma and urine using ELISA. The presence of uromodulin aggregates will be detected with Congo Red in urine and on kidney section as well as by measuring the molecular weight of uromodulin on Western blots.

It is expected that Gal3 antibodies will modulate kidney weight by at least 10% compared to isotype controls. It is expected that Gal3 antibodies will increase the volume of urine produced by at least 10% compared to isotype controls. It is expected that Gal3 antibodies will decrease the number or size of casts by at least 10% compared to isotype controls. It is expected that one or more of the following kidney injury biomarkers in plasma or urine will be modulated by at least 10% compared to isotype controls: N-GAL, KIM-1, IL-6, creatinine, glucose, albumin, BUN, K+, phosphorus, Na+, Ca+, tCO2.

### Example 19: Gal3 Antibodies Block Gal3-Induced Aggregation of IAPP

Islet Amyloid Polypeptide (IAPP) is secreted by pancreatic beta cells and normally modulates insulin activity in skeletal muscle, influencing energy homeostasis, satiety, blood glucose levels, adiposity, and body weight. When aggregated, it can be toxic to cells and promote insulin resistance and diabetes. To test whether anti-Gal3 antibodies could be utilized to inhibit IAPP-mediated disease, it was first established that Gal3 promotes IAPP aggregation. IAPP was mixed with recombinant human Gal3 and aggregation was measured with Western blots.

IAPP protein (Sigma-Aldrich #D2162) was diluted to a final concentration of 0.1 mg/ml by adding 10 mM sodium phosphate buffer, pH 7.4. Only 0.1 mg/mL IAPP, only 0.1 mg/mL rhGal3, or the combination of IAPP and rhGal3 were continuously stirred with a stir bar at room temperature, and aliquots were removed at 0, 0.5, 1, 2, 3, 4, and 5 hours of incubation and frozen at −20° C. for later analysis. To detect enhanced protein size seen in oligomers, samples were rapidly thawed at 37° C. and then 5 μL was loaded onto a 4-12% Criterion™ XT Bis-Tris Protein Gel (Bio-Rad #3450124), transferred to nitrocellulose (Amersham GE #10600001), and equal loading was confirmed with Ponceau Red staining. After the Ponceau Red was washed off, the nitrocellulose was blocked in 10% non-fat milk/TBS-Tween-20, and then incubated for 1 hour with I11 rabbit anti-amyloid oligomer primary antibody. After washing, the nitrocellulose was incubated for 1 hour with 1:5000 Donkey anti-rabbit HRP detection antibody (Jackson ImmunoResearch #711-035-152). After a final wash, bands were detected with a chemiluminescent substrate (1:1 mixture of Advansta Cat #R-03021-D10 and #R-03031-D10). Images were captured on an Azure imager.

IAPP protein (Sigma-Aldrich #D2162) was diluted to a final concentration of 0.1 mg/mL by adding 10 mM sodium phosphate buffer, pH 7.4. IAPP was incubated alone or with 100 μg/mL Gal3, and 100 μg/mL Gal3 alone was used as a control. The mixture was run on a Western blot with amyloid oligomer detected with the I11 antibody.

As seen in FIG. 9A, a ˜60 kDa band is observed for IAPP alone, indicating that this protein normally forms aggregates. The intensity of this band is increased when IAPP is mixed with Gal3 (FIG. 9B), indicating that more aggregation of IAPP occurs in the presence of Gal3.

FIG. 9C depicts samples of IAPP alone, Gal3 alone, or IAPP incubated with Gal3 at time points of 0, 0.5, 3, and 5 hours of incubation, showing that IAPP oligomerizes after 3 hours of incubation with Gal3. FIG. 9D is a quantification of the ˜60 kDa bands of FIG. 9C comparing the relative abundance of IAPP in each condition.

To identify anti-Gal3 antibodies with therapeutic potential, a screen of anti-Gal3 antibodies that block aggregation will be conducted. Anti-Gal3 antibodies or isotype controls will be co-incubated with 0.1 mg/mL IAPP with or without 0.1 mg/mL rhGal3, continuously stirring the mixture for up to 96 hours. Aggregation of IAPP will be detected by probing a Western blot with the I11 antibody. It is expected that antibodies that fully or partially block IAPP aggregation by at least 10% and those that do not affect it will be identified. Blockers can be sub-categorized further by epitope binning.

An ELISA was used to evaluate the binding of Gal3 to aggregated and unaggregated IAPP. To start the ELISA, coating solutions were prepared by diluting galectin-3 proteins (TrueBinding, QCB200349) (TrueBinding, QCB200352) (Biolegend, 599806) in separate volumes of PBS (Corning, 21-030-CM), each to a concentration of 10 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. IAPP solution for binding was prepared by diluting biotinylated aggregated IAPP in 2% BSA in PBST to a concentration of 10 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

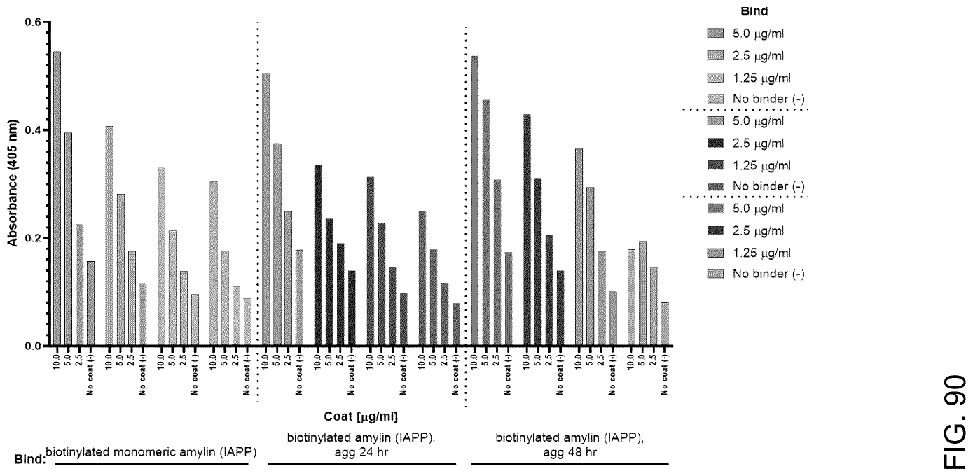

FIG. 90 depicts the results of the ELISA assay examining binding of hGal3 to aggregated and unaggregated IAPP. As can be seen in FIG. 90, IAPP is a weak binder of hGal3. Aggregation of IAPP causes a minor increase in Gal3 binding affinity.

### Example 20: Gal3 Antibodies Block IAPP Aggregate-Induced Cell Toxicity

IAPP aggregates can cause pancreatic beta cell death. To identify Gal3 antibodies that reduce beta cell death induced by IAPP aggregates, it first will be established that Gal3 promotes IAPP-induced cell death. Apoptosis in human INS-1 832/13 (Sigma-Aldrich #SCC207) and mouse MIN6 (ATCC) beta cell lines will be compared after treatment with IAPP pre-incubated with or without Gal3.

2×10⁵ cells will be grown overnight in a 96 well plate, and then cells will be stimulated with IAPP, IAPP pre-mixed with Gal3 to form aggregates, or Gal3 alone. Gal3 will be mixed on its own without IAPP as a control. 2-25 μL of pre-formed aggregates or control proteins will be taken out and incubated with the plated cells. After 8-48 hours, apoptosis will be measured by the Fluorometric Caspase 3 Assay Kit (Sigma-Aldrich #CASP3F) according to manufacturer's instructions and/or by flow cytometry using FITC Annexin V Apoptosis Detection kit with 7-AAD (Biolegend #640922). It is expected that cells incubated with the combination of Gal3 and IAPP will have at least 10% more death than cells incubated with IAPP or Gal3 alone.

To identify anti-Gal3 antibodies with therapeutic potential, a screen of anti-Gal3 antibodies that block cytotoxicity will be conducted. Various concentrations of antibodies will be incubated in the assays described above and attenuation of cell death will be assessed. Antibodies that block cytotoxicity by at least 10% will be identified. Blockers can be sub-categorized further by epitope binning.

### Example 21: Gal3 Antibodies Ameliorate IAPP-Induced Amyloidosis

Loss or dysfunction of pancreatic beta cells can lead to insulin and blood sugar dysregulation. IAPP aggregates are the major component of the cytotoxic amyloid deposits found in the pancreas of patients.

To identify anti-Gal3 antibodies that ameliorate disease caused by IAPP aggregates in vivo, toxic IAPP aggregates formed in vitro in the presence of Gal3 will be injected at different doses intra-peritoneally in mice. Blood sugar dysregulation will be monitored by standard glucose tolerance tests (GTT) and insulin tolerance tests (ITT assays). When dysregulation is evident, animals will be treated with Gal3 antibodies or remain untreated. It is expected that at least 10% improvement in GTT and ITT will be seen after treatment with antibodies compared to controls. Accumulation of IAPP aggregates in the pancreas will be assessed by IHC using Congo Red to stain aggregates. It is expected that at least a 10% reduction in the area of the sections that are positive for Congo Red will be seen.

In a second model of IAPP-mediated disease, RIPHAT mice (Jackson Labs, #008232) that express human islet amyloid polypeptide (h-IAPP) under the regulatory control of the rat insulin II promoter will be tested. Aged RIPHAT mice develop extracellular IAPP amyloid deposits associated with beta cell death and hyperglycemia. To test if anti-Gal3 antibodies can ameliorate disease caused by IAPP aggregates, aged mice will be treated with anti-Gal3 antibodies or left untreated. It is expected that at least 10% improvement in GTT and ITT will be seen after treatment with antibodies compared to controls. Accumulation of IAPP aggregates in the pancreas will be assessed by IHC, using Congo Red to stain aggregates. It is expected that at least a 10% reduction in the area of the sections that are positive for Congo Red will be seen.

### Example 22: Gal3 Antibodies Block Gal3-Induced Aggregation of SAA1

The Serum Amyloid Protein A (SAA) isoform-1 (SAA1) can aggregate, causing or exacerbating chronic inflammatory disorders such as rheumatoid diseases (e.g. arthritis, lupus), gastrointestinal diseases (e.g. Crohn's disease, ulcerative colitis), or persistent infections (e.g. tuberculosis, leprosy). The SAA isoform SAA2 does not tend to oligomerize and is not toxic. To test whether anti-Gal3 antibodies could be utilized to inhibit SAA amyloidosis, it will be first established that Gal3 promotes SAA aggregation. SAA (sequence similar to SAA2) or SAA1 will be mixed with recombinant human Gal3 and aggregation will be measured with Western and dot blots. Formation of fibrils (a class of aggregates) formation will be quantified using a Thioflavin T assay.

hSAA (ProSpecBio, #CYT-942) or hSAA1 (ProSpecBio, CYT-787) recombinant human protein will be mixed with continuous stirring at room temperature with or without Gal3 added. At 0 hours (immediately after mixing together), and at time points up to 363 hours, 2-10 μL aliquots will be removed and immediately frozen at −80° C. After all samples are collected, they will be analyzed by Western and dot blots.

To detect enhanced protein size seen in aggregates, samples will be rapidly thawed at 37° C. and then 2-5 μL will be loaded onto a 4-12% Criterion™ XT Bis-Tris Protein Gel (Bio-Rad #3450124), transferred to nitrocellulose (Amersham GE #10600001), and equal loading will be confirmed with Ponceau Red staining. After the Ponceau Red is washed off, the nitrocellulose will be blocked in 10% non-fat milk/TBS-Tween-20, then incubated for 1 hour with rabbit anti-SAA primary antibody (Abcam, #ab190801). After washing, the nitrocellulose will be incubated with Donkey anti-rabbit HRP detection antibody (Jackson ImmunoResearch #711-035-152). After a final wash, bands will be detected with a chemiluminescent substrate (1:1 mixture of Advansta #R-03021-D10 and #R-03031-D10). Images are captured on an Azure imager. SAA and SAA1 aggregates will be detected by their specific antibodies, but at a higher molecular weight on the gel than non-aggregated proteins. It is expected that Gal3 promotes the accumulation of higher molecular weight aggregates of SAA1, which are nearly undetectable when SAA1 is incubated alone.

In another method to detect aggregates, samples will be rapidly thawed at 37° C. and then 2-10 μL will be loaded onto a nitrocellulose membrane (1 membrane per primary) and air-dried for 1 hour at room temperature. Equal loading will be confirmed with Ponceau Red staining. After the Ponceau Red is washed off, the nitrocellulose will be blocked in 10% non-fat milk/TBS-Tween-20, and then incubated for 1 hour with either A11 anti-amyloid oligomer (Thermo-Fisher Scientific, #AHB0052), OC anti-amyloid oligomer (Sigma-Aldrich #AB2286) or anti-SAA primary antibodies (Abcam #ab190801). After washing, the nitrocellulose will be incubated for 1 hour with 1:5000 Donkey anti-rabbit HRP detection antibody (Jackson ImmunoResearch #711-035-152). After a final wash, dots will be detected with a chemiluminescent substrate (1:1 mixture of Advansta Cat #R-03021-D10 and #R-03031-D10). Images will be captured on an Azure imager and quantified. It is expected that Gal3 will increase the size of dots detected by A11 or OC by at least 10% compared to the same point where Gal3 is not added. It is expected that the SAA antibody, which serves as a loading control, will remain at the same level in all samples. This demonstrates that Gal3 promotes aggregation of SAA1.

When Thioflavin T binds to β-sheet-rich structures such as amyloid fibrils, it fluoresces at 485 nm. To measure fibrils, 20 μM of Thioflavin T will be mixed with 100 μM of hSAA (ProSpecBio, #CYT-942) or hSAA1 (ProSpecBio, CYT-787) recombinant human protein with or without 300 μM of rhGal3 in a final volume of 100 μL in a black 96-well plate. Controls include rhGal3 and Thioflavin T without SAA or SAA1 to ensure specific binding to amyloid fibrils. Thioflavin T incubated alone will also be included for background subtraction from final readings. Each plate will contain duplicate conditions. Two identical plates will be set up, one to be incubated at room temperature and one at 37° C. on a rotor shaking at 600 rpm. Fluorescence will be read at various time points up to 363 hours on a SpectraMax plate reader machine at excitation 450 nm and emission 485 nm. To quantify fibril formation, fluorescence at 37° C. minus background fluorescence will be calculated. Conditions that demonstrate increased fluorescence will indicate enhanced fibril formation. It is expected that Gal3 increases fluorescence by at least 10% when added, demonstrating that it promotes SAA1 fibril formation.

To identify anti-Gal3 antibodies with therapeutic potential, a screen of anti-Gal3 antibodies that block aggregation or fibril formation will be conducted. Time points demonstrating aggregation in the presence of Gal3 will be chosen from the Western blots, dot blots, and Thioflavin T assays. Various concentrations of antibodies will be incubated with the assays described above to detect antibodies that block aggregation or fibril formation. It is expected that antibodies that block SAA1 aggregation by at least 10% and those that do not affect aggregation will be identified. Blockers can be sub-categorized further by epitope binning.

### Example 23: Gal3 Antibodies Block SAA1 Aggregate-Induced Cell Toxicity

SAA1 aggregates can cause cell death, which can damage the organs they accumulate in and lead to chronic inflammatory disorders. To test whether anti-Gal3 antibodies could be utilized to inhibit SAA amyloidosis, it will be first established if SAA aggregates formed in the presence of Gal3 can lead to cell toxicity. Apoptosis in HEK293T cells will be compared after treatment with SAA and SAA1 peptides pre-incubated with or without Gal3.

2×10⁵ HEK293T cells will be grown overnight in a 96 well plate, and then cells will be stimulated with 2-25 μL SAA1 alone, SAA1 pre-mixed with Gal3 to form aggregates, or Gal3 alone in serum-free DMEM media. After 6 hours, cytotoxicity will be measured by lactate dehydrogenase (LDH) release according to manufacturer's instructions using the CytoTox 96® Non-Radioactive Cytotoxicity Assay (Promega #G1780). Minimal apoptosis will be observed with SAA, SAA plus Gal3, or SAA1 alone. It is expected that SAA1 pre-incubated with Gal3 will increase apoptosis by at least 10%.

To identify anti-Gal3 antibodies with therapeutic potential, a screen of anti-Gal3 antibodies that block cytotoxicity will be conducted. Various concentrations of antibodies will be incubated in the assays described above and attenuation of cell death will be assessed. It is expected that antibodies that block cytotoxicity by at least 10% and those that do not affect cytotoxicity will be identified. Blockers can be sub-categorized further by epitope binning.

### Example 24: Gal3 Antibodies Ameliorate Chronic Inflammation in Autoimmune Disease

To test if anti-Gal3 antibodies can attenuate inflammatory disease, mouse models of rheumatoid disease and inflammatory bowel disease (IBD) will be treated.

In a model of rheumatoid arthritis, wild-type and IL1ra knockout mice (Jackson Labs #25391376) are injected with SAA that was aggregated in vitro and 2% AgNO3. Mice will then be treated with anti-Gal3 antibodies or isotype controls. Joint inflammation will be quantified by arthritic score based on visual assessment or caliper measurement. Systemic inflammation will be quantified by the levels of TNFα, IL-1β, IL-6, CRP or other pro-inflammatory biomarkers in the plasma. Aggregate deposition will be quantified in spleen, liver, kidneys, heart, thyroid glands, intestines, adrenal glands or other organs using Congo Red. It is expected that mice that receive SAA aggregates will have increased symptoms of disease and evidence of pathophysiology not seen in animals that do not received aggregates. It is expected that in animals that will receive the SAA aggregates, Gal3 antibody-treated animals will have at least a 10% reduction in arthritic score compared to isotype controls. It is expected that Gal3 antibody-treated animals will have at least a 10% reduction in at least one biomarker of inflammation compared to isotype controls. It is expected that SAA aggregate deposition will be decreased by at least 10% in at least one organ compared to isotype controls.

In a model of IBD, SAA aggregates will be detected in IL-2−/− mice before symptom development and it is expected that the level of aggregates will correlate to severity of disease. To test the effect of Gal3 therapeutic antibodies, wild-type, IL-2+/− and IL-2−/− mice (Jackson Labs #002229) will be injected with Gal3 antibodies or isotype controls. IBD will be quantified by scoring IHC sections for leukocyte infiltration and epithelial hyperplasia. Systemic inflammation will be quantified by the levels of TNFα, IL-1β, IL-6, CRP or other pro-inflammatory biomarkers in the plasma. Aggregate deposition will be quantified in spleen, liver, kidneys, heart, thyroid glands, intestines, adrenal glands or other organs using Congo Red. It is expected that Gal3 antibody-treated animals will have at least a 10% reduction in IBD score compared to isotype controls. It is expected that Gal3 antibody-treated animals will have at least a 10% reduction in at least one biomarker of inflammation compared to isotype controls. It is expected that SAA aggregate deposition will be decreased by at least 10% in at least one organ compared to isotype controls.

In a second model of IBD, IL-2+/− mice will be left untreated or injected with SAA that was aggregated in vitro to enhance SAA-induced symptom development. To test the effect of Gal3 therapeutic antibodies, untreated or injected mice will be treated with Gal3 antibodies or isotype controls. IBD will be quantified by scoring IHC sections for leukocyte infiltration and epithelial hyperplasia. Systemic inflammation will be quantified by the levels of TNFα, IL-1β, IL-6, CRP or other pro-inflammatory biomarkers in the plasma. Aggregate deposition will be quantified in spleen, liver, kidneys, heart, thyroid glands, intestines, adrenal glands or other organs using Congo Red. It is expected that Gal3 antibody-treated animals will have at least a 10% reduction in IBD score compared to isotype controls. It is expected that Gal3 antibody-treated animals will have at least a 10% reduction in at least one biomarker of inflammation compared to isotype controls. It is expected that SAA aggregate deposition will be decreased by at least 10% in at least one organ compared to isotype controls.

### Example 25: Gal3 Antibodies Block Gal3-Induced Aggregation of p53

Misfolding and aggregation of the tumor suppressor p53 can play a role in cancer development. To test whether anti-Gal3 antibodies could be utilized to inhibit cancer, it will be first established that Gal3 promotes p53 aggregation. p53 will be mixed with recombinant human Gal3 and aggregation will be measured with Western and dot blots. Formation of fibrils (a class of aggregates) formation will be quantified using a Thioflavin T assay.

Recombinant p53 human protein (Biorbyt #orb418963) will be mixed with continuous stirring at room temperature with or without Gal3 added. At 0 hours (immediately after mixing together), and at time points up to 363 hours, 2-10 μL aliquots will be removed and immediately frozen at −80° C. After all samples are collected, they will be analyzed by Western and dot blots.

To detect enhanced protein size seen in aggregates, samples will be rapidly thawed at 37° C. and then 2-5 μL will be loaded onto a 4-12% Criterion™ XT Bis-Tris Protein Gel (Bio-Rad #3450124), transferred to nitrocellulose (Amersham GE #10600001), and equal loading will be confirmed with Ponceau Red staining. After the Ponceau Red is washed off, the nitrocellulose will be blocked in 10% non-fat milk/TBS-Tween-20, and then incubated for 1 hour with rabbit anti-p53 primary antibody (R&D Systems MAB1355). After washing, the nitrocellulose will be incubated with anti-mouse HRP detection antibody (Abcam #ab6789). After a final wash, bands will be detected with a chemiluminescent substrate (1:1 mixture of Advansta #R-03021-D10 and #R-03031-D10). Images will be captured on an Azure imager. p53 aggregates will be detected by their specific antibodies, but at a higher molecular weight on the gel than non-aggregated proteins. It is expected that Gal3 promotes the accumulation of higher molecular weight aggregates of p53, which are nearly undetectable when p53 is incubated alone.

In another method to detect aggregates, samples will be rapidly thawed at 37° C. and then 2-10 μL will be loaded onto a nitrocellulose membrane (1 membrane per primary) and air-dried for 1 hour at room temperature. Equal loading will be confirmed with Ponceau Red staining. After the Ponceau Red is washed off, the nitrocellulose will be blocked in 10% non-fat milk/TBS-Tween-20, then incubated for 1 hour with either A11 anti-amyloid oligomer (Thermo-Fisher Scientific, #AHB0052), OC anti-amyloid oligomer (Sigma-Aldrich #AB2286) or anti-p53 primary antibodies (R&D Systems MAB1355). After washing, the nitrocellulose will be incubated for 1 hour with 1:5000 Donkey anti-rabbit HRP detection antibody (Jackson ImmunoResearch #711-035-152) or anti-mouse HRP detection antibody Abcam #ab6789). After a final wash, dots will be detected with a chemiluminescent substrate (1:1 mixture of Advansta Cat #R-03021-D10 and #R-03031-D10). Images will be captured on an Azure imager and quantified. It is expected that Gal3 increases the size of dots detected by A11 or OC by at least 10% compared to the same point where Gal3 is not added. It is expected that the p53 antibody, which serves as a loading control, remains at the same level in all samples. This demonstrates that Gal3 promotes aggregation of p53.

When Thioflavin T binds to β-sheet-rich structures such as amyloid fibrils, it fluoresces at 485 nm. To measure fibrils, 20 μM of Thioflavin T will be mixed with 100 μM of p53 recombinant human protein with or without 300 μM of rhGal3 in a final volume of 100 μL in a black 96-well plate. Controls will include Gal3 and Thioflavin T without p53 to ensure specific binding to amyloid fibrils. Thioflavin T incubated alone will also be included for background subtraction from final readings. Each plate will contain duplicate conditions. Two identical plates will be set up, one to be incubated at room temperature and one at 37° C. on a rotor shaking at 600 rpm. Fluorescence will be read at various time points up to 363 hours on a SpectraMax plate reader machine at excitation 450 nm and emission 485 nm. To quantify fibril formation, fluorescence at 37° C. minus background fluorescence will be calculated. Conditions that demonstrate increased fluorescence will indicate enhanced fibril formation. It is expected that Gal3 increases fluorescence by at least 10% when added, demonstrating that it promotes p53 fibril formation.

To identify anti-Gal3 antibodies with therapeutic potential, a screen of anti-Gal3 antibodies that block aggregation or fibril formation will be conducted. Time points demonstrating aggregation in the presence of Gal3 will be chosen from the Western blots, dot blots, and Thioflavin T assays. Various concentrations of antibodies will be incubated with the assays described above to detect antibodies that block aggregation or fibril formation. It is expected that antibodies that block p53 aggregation by at least 10% and those that do not affect aggregation will be identified. Blockers can be sub-categorized further by epitope binning.

### Example 26: Gal3 Antibodies Block p53 Aggregate-Induced Cell Toxicity

p53 aggregates taken up by cells can seed the misfolding and inactivation of cytoplasmic p53, leading to cell death. To identify Gal3 antibodies that promote cancer cell death opposed by p53 aggregation, we first establish that Gal3 reduces p53-induced cell death. A variety in cancer cell lines, including but not limited to SH-SY5Y neuroblastoma, MCF7 and MD-MBA231 breast cancer cells, will be assessed.

Cells will be grown overnight in a 96 well plate, and then cells will be stimulated with p53, p53 pre-mixed with Gal3 to form aggregates, or Gal3 alone. Gal3 will be mixed on its own without p53 as a control. 2-25 μL will be taken out and incubated with the plated cells. After 8-48 hours, viability will be measured by the CYQUANT MTT assay (Thermo-Fisher #V13154), according to the manufacturer's instructions. The absorbance at 560 nm and background scattering at 690 nm will be measured in a SpectraMax plate reader (Molecular Devices). The absorbance values at 560 nm will be used to calculate the viability of cells in comparison to buffer control. It is expected that viability will be reduced by at least 10% when p53 aggregated with Gal3 is added compared to p53 alone in an unaggregated form or Gal3 alone.

To identify anti-Gal3 antibodies with therapeutic potential, a screen of anti-Gal3 antibodies that promote cytotoxicity will be conducted. Various concentrations of antibodies will be incubated in the assays described above and promotion of cell death will be assessed. It is expected that antibodies that promote cytotoxicity by at least 10% will be identified. These antibodies can be sub-categorized further by epitope binning.

### Example 27: Anti-Gal3 Antibodies for Use in the Treatment of a Proteopathy or Amyloidosis

Patients present with a proteopathy, such as an amyloid proteopathy, or amyloidosis, such as a synucleinopathy, Parkinson's disease, dementia with Lewy bodies, multiple system atrophy, tauopathy, Alzheimer's disease, progressive supranuclear palsy, corticobasal degeneration, Pick's disease, TDP-43 proteopathy, amyotrophic lateral sclerosis, frontotemporal lobar degeneration, TTR amyloidosis, cardiac amyloidosis, uromodulin-associated kidney disease, IAPP amyloidosis, SAA amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation, or diseases caused by dysfunction of α-synuclein, tau protein, TDP-43, transthyretin, uromodulin, IAPP, SAA, or p53. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses in at an amount of 1 ng (or in the alternative: 0.1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or any time within a range defined by any two of the aforementioned times).

The anti-Gal3 antibody is repeatedly administered until the patient experiences a reduction or amelioration of the proteopathy or amyloidosis. The reduction or amelioration of the proteopathy or amyloidosis may be detected through diagnostic methods generally known in the art, including but not limited to a biopsy, blood or urine test, echocardiogram, or technetium pyrophosphate scintigraphy. The reduction or amelioration of the proteopathy or amyloidosis may be reduced by at least 5% (or in the alternative: 10%, 15%, 20%, 25%, 30%, 35%, 40%, 45%, 50%, 55%, 60%, 65%, 70%, 75%, 80%, 85%, 90%, 95%, or 99%) after the administering step relative to the proteopathy or amyloidosis prior to the administering step, as assessed by a qualified individual.

### Example 28: Gal3 Promotes Apolipoprotein E Oligomerization

Gal3 was tested for its ability to promote oligomerization of apolipoprotein E (APOE). Recombinant forms of different polymorphic alleles of APOE (APO-E2, APO-E3, and APO-E4) were incubated with Gal3 at room temperature for 0, 1, 2, 3, 4, 5, or 24 hours. APOE only and Gal3 only preparations were used as control.

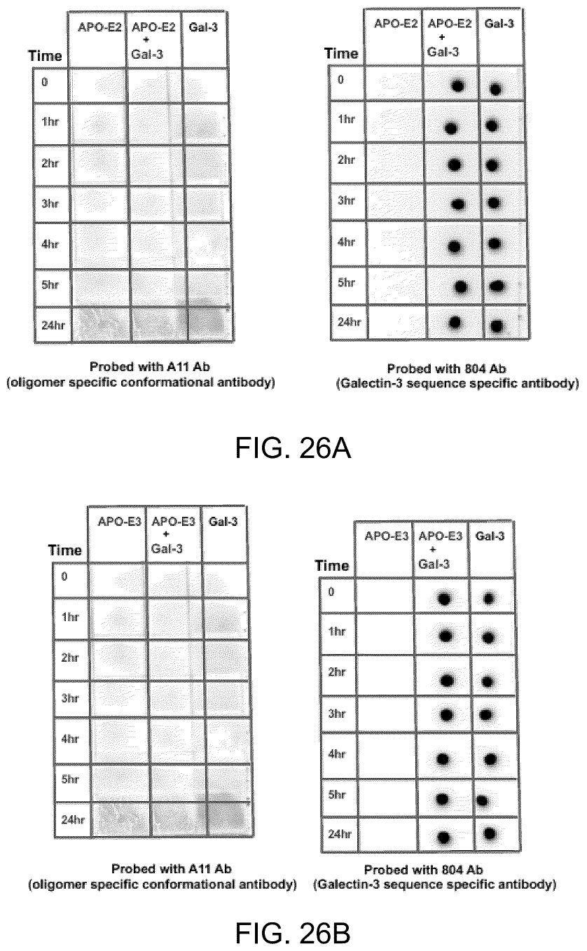

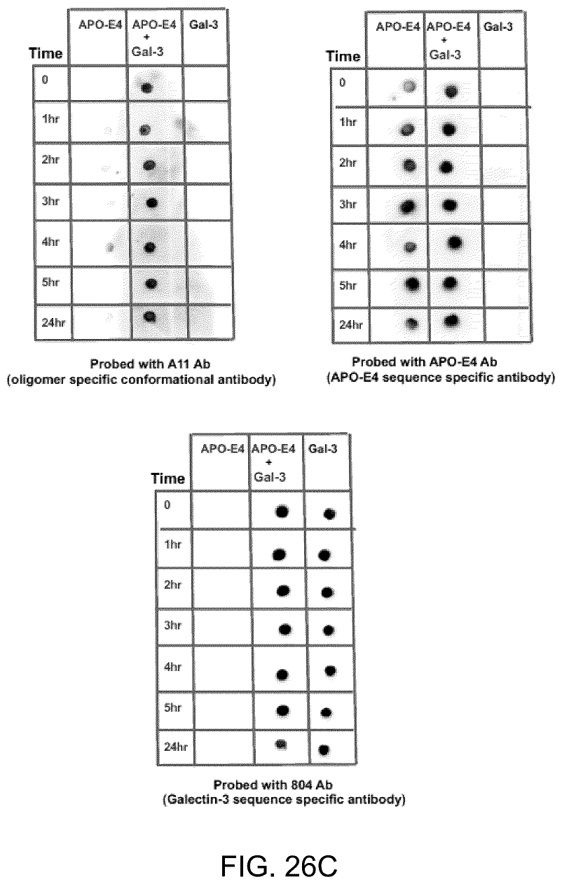

Dot blots of the APOE/Gal3 mixtures and controls were performed and probed with A11 antibody and anti-Gal3804 antibody. FIG. 26A-B show that for APOE alleles APO-E2 and APO-E3, no oligomers were detected. However, FIG. 26C shows that Gal3 promoted APO-E4 oligomerization at the time points tested as indicated by positive signal when probed with A11.

An ELISA was also used to detect Gal3 binding to APOE-4. Prior to the ELISA, human APOE-4 was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions. To start the ELISA, coating solutions were prepared by diluting galectin-3 proteins (TrueBinding, QCB200349) (TrueBinding, QCB200352) (Biolegend, 599806) in separate volumes of PBS (Corning, 21-030-CM), each to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. APOE-4 solution for binding was prepared by diluting biotinylated aggregated APOE-4 in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

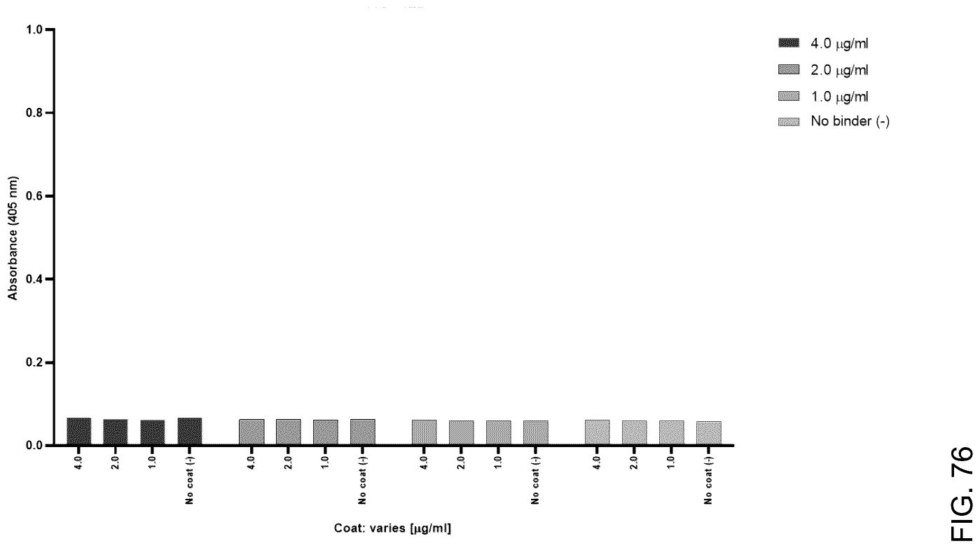

FIG. 76 depicts the results of the ELISA assay examining binding of hGal3 to aggregated ApoE4 protein. As can be seen in FIG. 76, no binding between aggregated APOE-4 and Gal3 was observed.

### Example 29: Anti-Gal3 Antibodies Degrade Toxic Apolipoprotein E Oligomers

The exemplary anti-Gal3 antibody TB006 was tested for its ability to degrade APOE-4 oligomers. APOE-4/Gal3 mixtures were prepared, with APOE-4 only as control. The APOE-4 peptide was diluted to final concentration 0.1 mg/ml by adding 10 mM sodium phosphate buffer, pH 7.4. To investigate the effect of recombinant human Gal-3 (rhGal-3) on aggregation of APOE-4, 100 μg/ml of APOE-4 peptide was mixed rhGal-3 protein (100 μg/ml). The solution was continuously stirred during the aggregation time course using a stir bar for 24 hrs at RT. The aggregation profile of APOE4 peptide with out and with rhGal-3 probed with A11 & APOE4 sequence dependent antibody (Syn1) was then determined. These were then combined with TB006 (or MOPC21 as isotype control) at 0, 3, 10, or 100 μg of antibody, and incubated with the antibody for 0, 1, 2, or 3 hours at room temperature.

After incubating with respective secondary antibodies for 1 h, images were developed using Azure image developer.

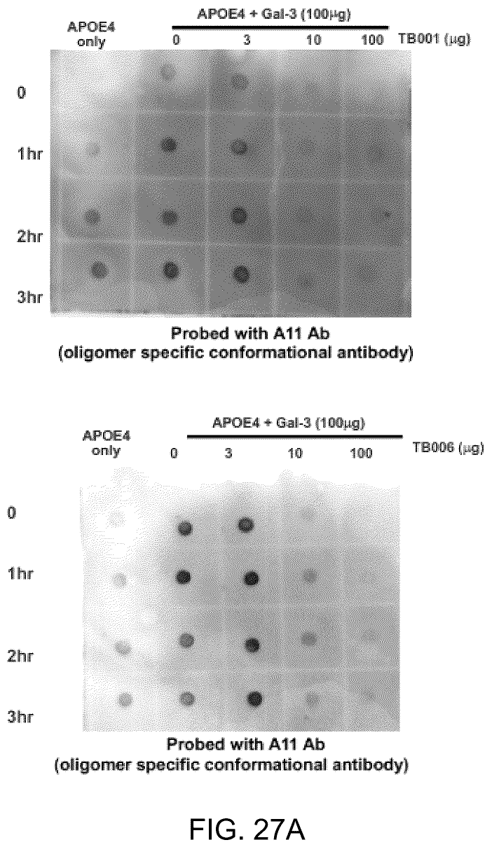

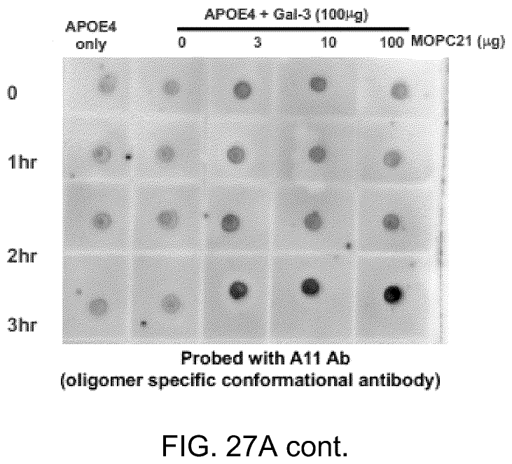

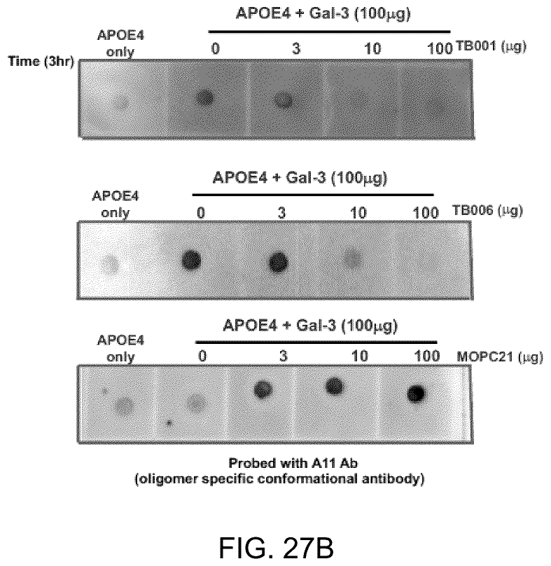

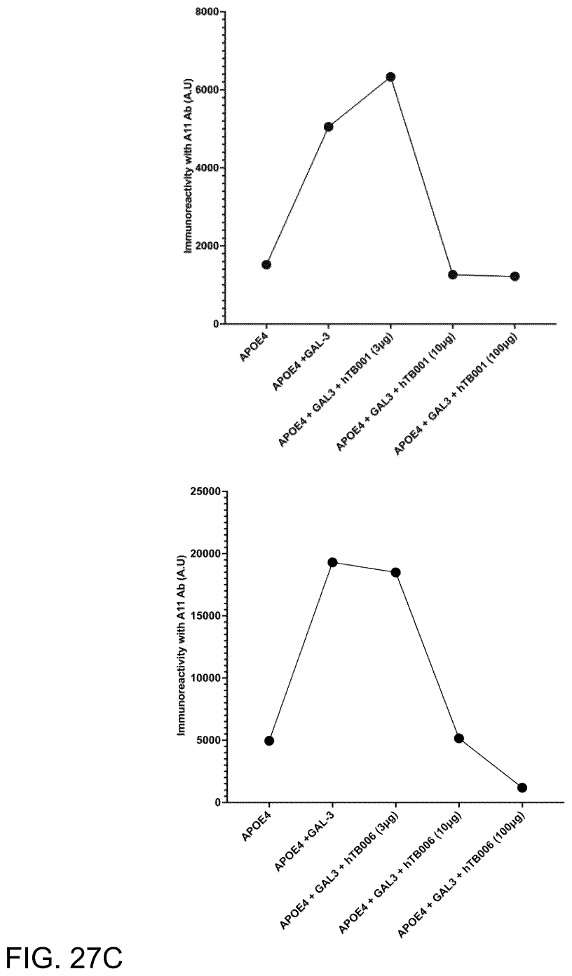

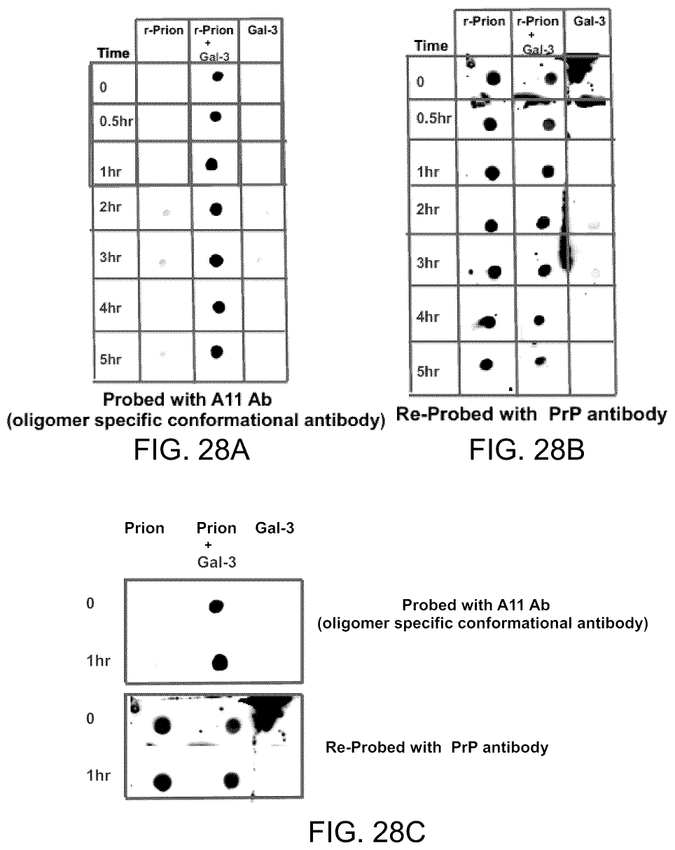

As shown in FIG. 27A, TB006 showed a dose-dependent ability to degrade APO-E4 oligomers in the APO-E4/Gal3 mixtures whereas MOPC21 control had no effect.

FIG. 27B shows the 3 hour time point of FIG. 27B. FIG. 27C is the quantification of the TB006-treated 3 hour time points of FIG. 27B based on signal intensity.

### Example 30: Gal3 Promotes Prion Protein Oligomerization

Gal3 was tested for its ability to promote oligomerization of prion protein. Recombinant prion protein was incubated with Gal3 at room temperature for 0, 0.5, 1, 2, 3, 4, or 5 hours. Prion protein only and Gal3 only preparations were used as control.

Dot blots of the prion protein/Gal3 mixtures and controls were performed and probed with A11 antibody and prion protein (PrP)-specific antibody. FIG. 25 shows that prion protein incubated with Gal3 formed oligomers at the time points tested as indicated by positive signal when probed with A11.

Gal3 binding to aggregated prion protein was also tested using an ELISA. Prior to the ELISA, human prion protein was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Prion protein solution for binding was prepared by diluting biotinylated aggregated prion protein in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

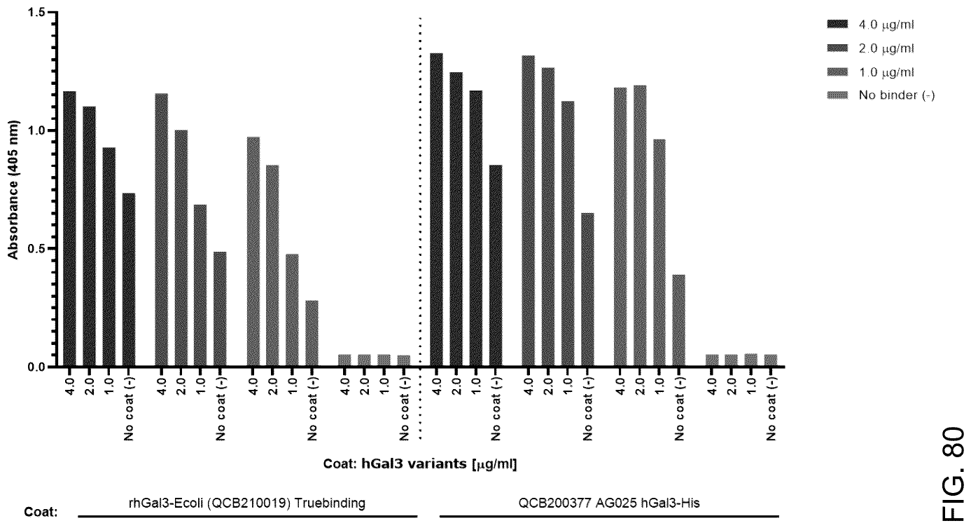

FIG. 80 depicts the results of the ELISA assay examining binding of hGal3 to aggregated prion protein. As can be seen in FIG. 80, hGal3 demonstrates strong binding to aggregated prion protein even at diminishing concentrations of aggregated prion protein or hGal3.

An ELISA was used to evaluate the blocking efficacy of various antibodies against hGal3 binding to aggregated prion protein. Prior to the ELISA, human prion protein was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Human Galectin-3 (Gal3) protein (TrueBinding, QCB200377) was diluted in PBS to a concentration of 10 μg/ml and coated a 96-well ELISA plate by applying 35 μl to each well. After incubating the plate at 4° C. O/N, the plate was washed three times with 300 μl PBST. The plate was then blocked for an hour with 150 μl of 2% BSA in PBST at RT with gentle rocking. Thereafter, the 2% BSA in PBST was discarded and 30 μl of TB001 (TrueBinding, QC190118), TB006 (TrueBinding, QC200208), several 20H5 (TrueBinding, various QC #), or Synagis hIgG4 Isotype (TrueBinding, QC190234) (3-fold serial dilutions beginning at 10 μg/ml) in 2% BSA in PBST was added to the wells, immediately followed by the addition of 30 μl of 1 μg/ml of biotinylated prion protein in 2% BSA in PBST. The plate was incubated for an hour at RT with gentle rocking and then washed three times with 300 μl PBST. Thereafter, Avidin HRP (Biolegend, 405103) was diluted in 2% BSA in PBST (1:2000 dilution) and 25 μl was added to the wells. The plate was incubated at RT for an hour with gentle rocking and washed three times with 300 μl PBST. 50 μl of ABTS (Life Technologies, 00-2024) was added to each well to develop, then the plate was read using a plate reader at an absorbance of 405 nm. Data was graphed using GraphPad Prism 8.0 software (GraphPad Software Inc).

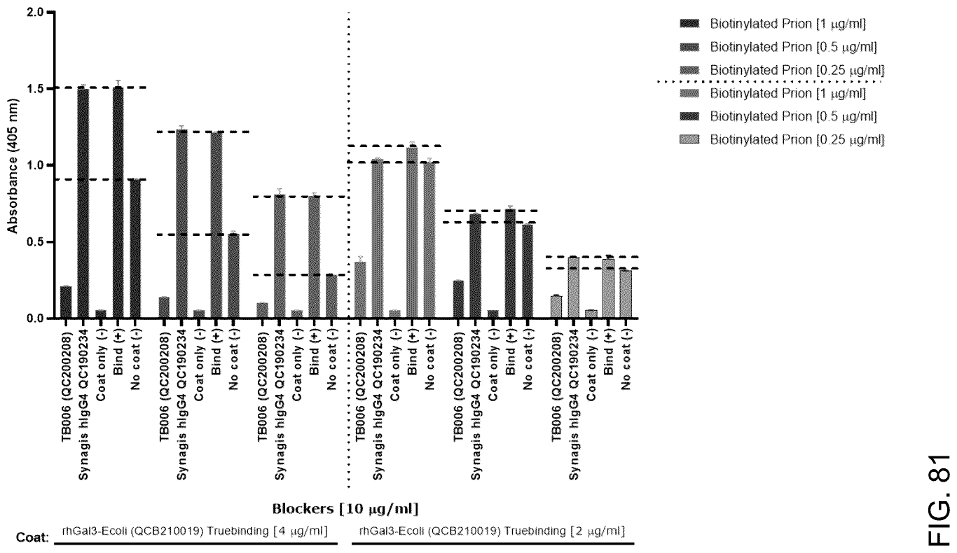

FIG. 81 depicts the results of the ELISA assay examining blocking efficacy of various antibodies against binding of hGal3 to aggregated prion protein. As can be seen in FIG. 81, various antibodies demonstrated significant blocking of hGal3 binding to aggregated prion protein even at diminishing concentrations of aggregated prion protein or hGal3.

### Example 31: Gal3 Promotes Neurofilament Light (NFL) Oligomerization

Gal3 was tested for its ability to promote oligomerization of neurofilament light (NFL) protein. Recombinant NFL was incubated with Gal3 at room temperature for 0, 1, 2, 3, 4, 5, or 24 hours. NFL only and Gal3 only preparations were used as control.

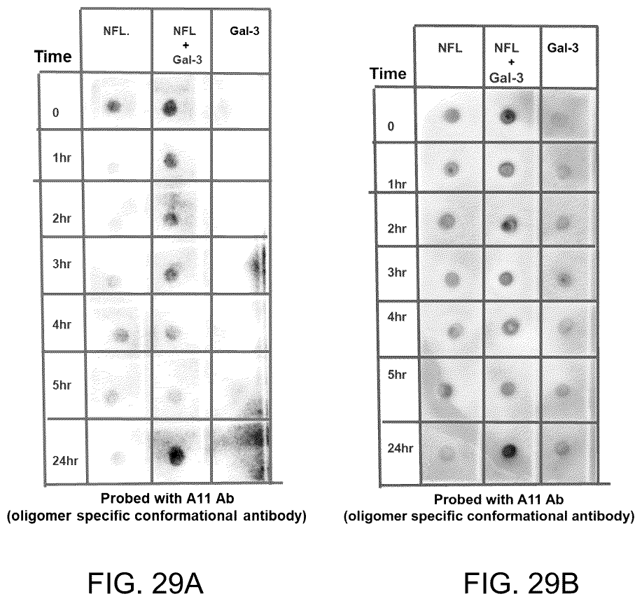

A dot blot of the NFL/Gal3 mixtures and controls were performed and probed with A11 antibody. FIG. 29A-B depicts promotion of aggregation of neurofilament light (NFL) protein by Gal3 as detected by dot blot.

To test whether anti-Gal3 antibodies could be utilized to inhibit proteopathies, it was first established using an ELISA that Gal3 is involved in the formation of aggregated NFL. Prior to the ELISA, human NFL was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. NFL solution for binding was prepared by diluting biotinylated aggregated NFL in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

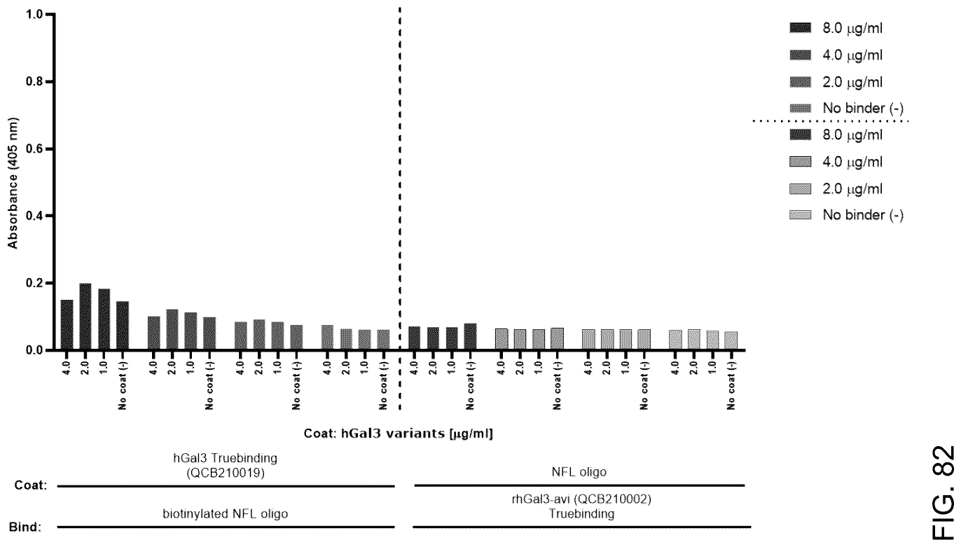

FIG. 82 depicts the results of the ELISA assay examining binding of hGal3 to aggregated NFL. As can be seen in FIG. 82, weak binding of hGal3 to aggregated NFL was observed.

### Example 32: Gal3 Promotes Aggregation of Aβ42

Deposition of Aβ42 plaques is a characteristic hallmark of Alzheimer's disease and cerebral amyloid angiopathy (CAA). To test whether anti-Gal3 antibodies could be utilized to inhibit Alzheimer's disease or CAA, it was first established that Gal3 promotes Aβ42 aggregation.

To test whether anti-Gal3 antibodies could be utilized to inhibit proteopathies, it was first established using dot blot that Gal3 is involved in the formation of oligomerized Aβ42.1 mg of lyophilized Aβ42 peptide (r-peptide) was resuspended in 90 μl of 100 mM NaOH and incubated for 10 minutes. The solution was then diluted to final concentration 0.1 mg/ml by adding 100 mM sodium phosphate buffer, pH 7.4. Synthetic Aβ42 peptide was then mixed with rhGal-3 protein, and incubated for 1 hr at RT. After 1 hour, different concentrations of hTB001 and hTB006Ab were added and the solution was incubated for 5 hours at RT. 2 μl of each sample was pipetted onto a Whatman nitrocellulose membrane at the appropriate time points (time 0-5 hr) for a dot blot and probed with antibody A11. In order to confirm the Aβ42, the membrane was also probed with antibody 6E10. After incubating with respective secondary antibodies for 1 h, images were developed using Azure image developer.

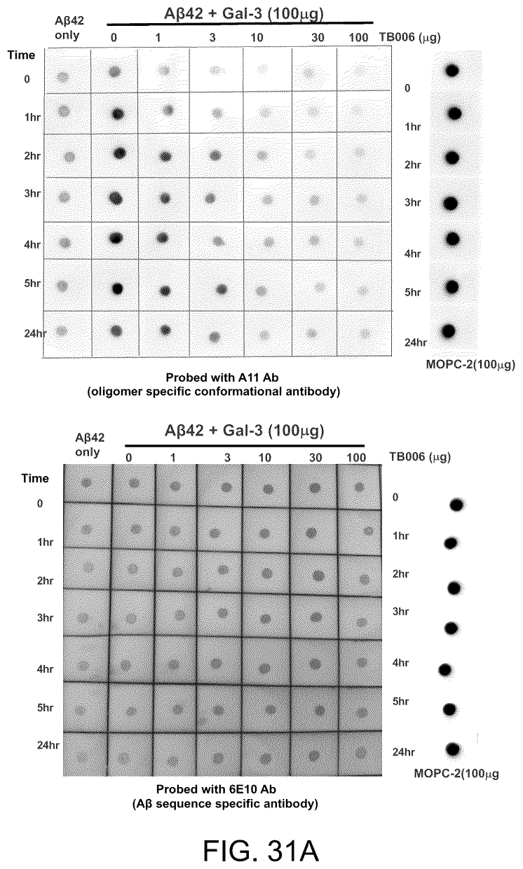

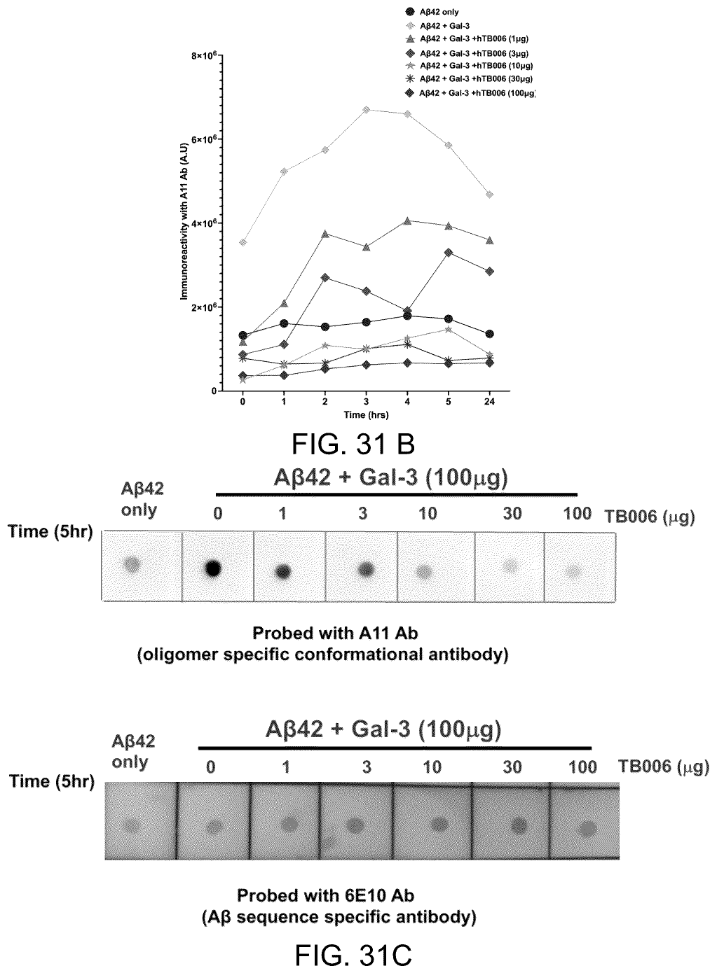

As can be seen in FIG. 31, rhGal-3 induces oligomerization of Aβ42. FIG. 31A-D depict degradation of toxic Aβ42 oligomers as detected by dot blot. FIG. 31A depicts 24 hour time course degradation of toxic Aβ42 oligomers by hTB006 probed with A11 and 6E10 antibody. FIG. 31B depicts quantification of Aβ42 oligomer degradation over the 24 hour time course. FIG. 31C depicts 5 hour time course degradation of toxic Aβ42 oligomers by hTB006 probed with A11 and 6E10 antibody. FIG. 31D depicts quantification of the effect of different concentrations of hTB006 on Gal-3 induced Aβ42 oligomers.

To determine whether oligomerized Aβ42 binds hGal3, Gal3 binding to oligomerized Aβ2 was also tested using an Elisa. Prior to the ELISA, human prion protein was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Prion protein solution for binding was prepared by diluting biotinylated aggregated Aβ42 in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

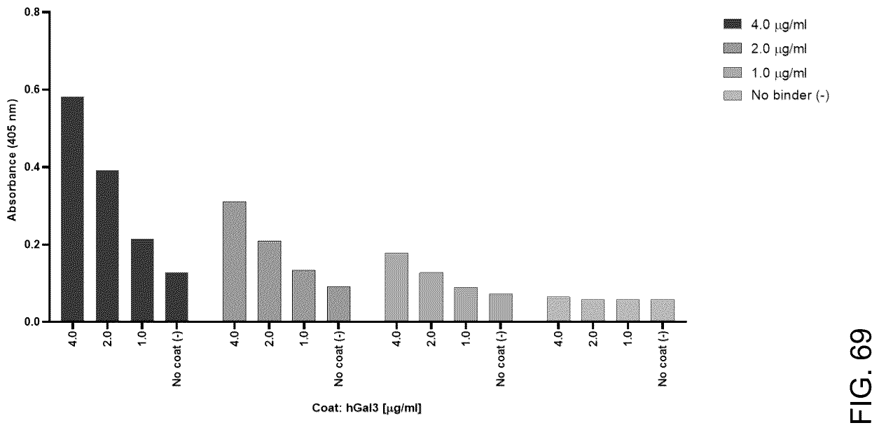

FIG. 69 depicts the results of the ELISA assay examining binding of hGal3 to aggregated Aβ42 protein. As can be seen in FIG. 69, hGal3 demonstrates moderate binding to Aβ42 even at diminishing concentrations of Aβ42 or hGal3.

### Example 33: Gal3 Promotes Aggregation of Aβ40

Deposition of Aβ40 plaques is a late stage characteristic hallmark of Alzheimer's disease and cerebral amyloid angiopathy (CAA). To test whether anti-Gal3 antibodies could be utilized to inhibit Alzheimer's disease or CAA, it was first established that Gal3 promotes Aβ40 aggregation.

To determine whether oligomerized Aβ40 binds hGal3, Gal3 binding to oligomerized Aβ40 was tested using a dot blot. 1 mg of lyophilized Aβ40 peptide was resuspended in 90 μl of 100 mM NaOH and incubated for 10 minutes. The solution was then diluted to final concentration 0.1 mg/ml by adding 100 mM sodium phosphate buffer, pH 7.4. Synthetic Aβ40 peptide was then mixed with rhGal-3 protein, and incubated for 1 hr at RT. After 1 hour, different concentrations of hTB001 and hTB006Ab were added and the solution was incubated for 5 hours at RT. 2 μl of each sample was pipetted onto a Whatman nitrocellulose membrane at the appropriate time points (time 0-5 hr) for a dot blot and probed with antibody A11. In order to confirm the Aβ40, the membrane was also probed with antibody 6E10. After incubating with respective secondary antibodies for 1 h, images were developed using Azure image developer.

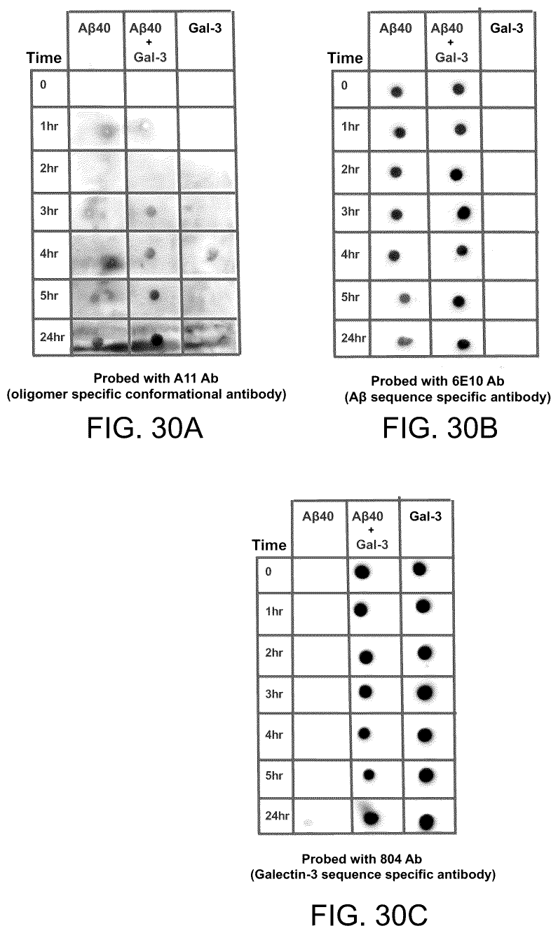

As can be seen in FIG. 30, rhGal-3 induces oligomerization of A340. FIG. 30A-C depict oligomerization of Aβ40 as detected by dot blot. FIG. 30A depicts 24 hour time course oligomerization of Aβ40 oligomers probed with A11 antibody. FIG. 30B depicts 24 hour time course oligomerization of Aβ40 oligomers probed with antibody 6E10. FIG. 30C depicts 24 hour time course oligomerization of Aβ40 oligomers probed with Gal3 sequence 804 antibody.

To determine whether oligomerized Aβ40 binds hGal3, Gal3 binding to oligomerized Aβ0 was also tested using an Elisa. Prior to the ELISA, human prion protein was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Prion protein solution for binding was prepared by diluting biotinylated aggregated Aβ40 in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

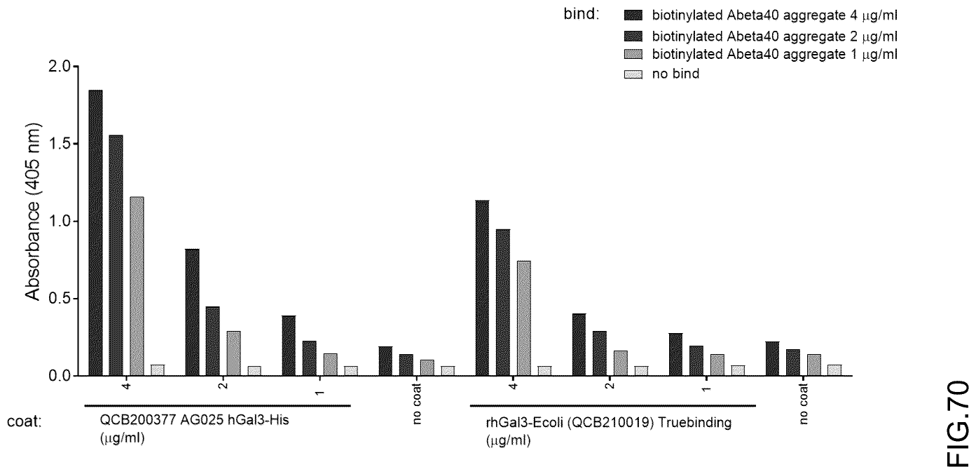

FIG. 70 depicts the results of the ELISA assay examining binding of hGal3 to aggregated Aβ40. As can be seen in FIG. 69, hGal3 demonstrates moderate binding to Aβ40 even at diminishing concentrations of Aβ40 or hGal3.

### Example 34: Gal3 Antibodies Block Gal3 Binding to Aβ40

An ELISA was performed to establish determine the blocking efficacy of various antibodies against hGal3 binding to Aβ40. Prior to the ELISA, human amyloid-beta 40 (Aβ340) was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Human Galectin-3 (Gal3) protein (TrueBinding, QCB200377) was diluted in PBS to a concentration of 4 μg/ml and coated a 96-well ELISA plate by applying 35 μl to each well. After incubating the plate at 4° C. O/N, the plate was washed three times with 300 μl PBST. The plate was then blocked for an hour with 150 μl of 2% BSA in PBST at RT with gentle rocking. Thereafter, the 2% BSA in PBST was discarded and 30 μl of TB001 (TrueBinding, QC190118), TB006 (TrueBinding, QC200208), several 20H5 (TrueBinding, various QC #), or Synagis hIgG4 Isotype (TrueBinding, QC190234) (3-fold serial dilutions beginning at 10 μg/ml) in 2% BSA in PBST was added to the wells, immediately followed by the addition of 30 μl of 1 μg/ml of biotinylated Aβ40 in 2% BSA in PBST. The plate was incubated for an hour at RT with gentle rocking and then washed three times with 300 μl PBST. Thereafter, Avidin HRP (Biolegend, 405103) was diluted in 2% BSA in PBST (1:2000 dilution) and 25 μl was added to the wells. The plate was incubated at RT for an hour with gentle rocking and washed three times with 300 μl PBST. 50 μl of ABTS (Life Technologies, 00-2024) was added to each well to develop, then the plate was read using a plate reader at an absorbance of 405 nm. Data was graphed using GraphPad Prism 8.0 software (GraphPad Software Inc).

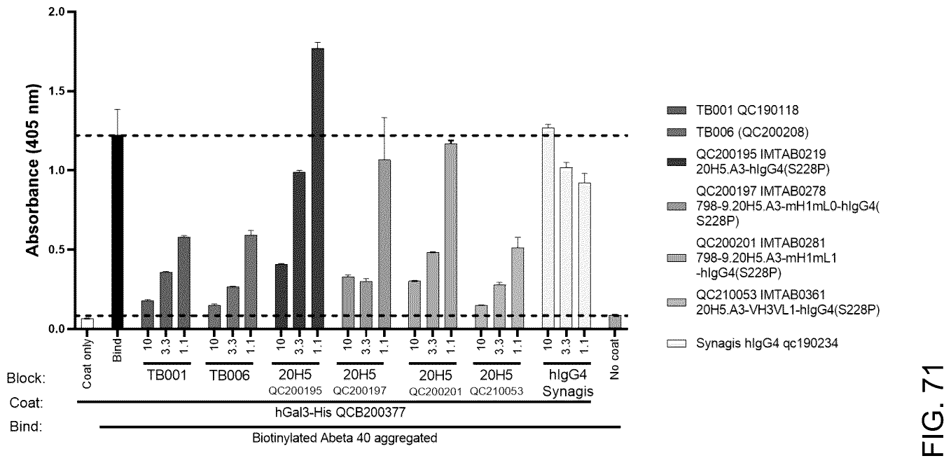

FIG. 71 depicts the results of the ELISA blocking assay. As can be seen in FIG. 71, various anti-Gal3 antibodies including TB001, TB006, and 20H5, displayed a significant ability to block hGal3 binding to Aβ40.

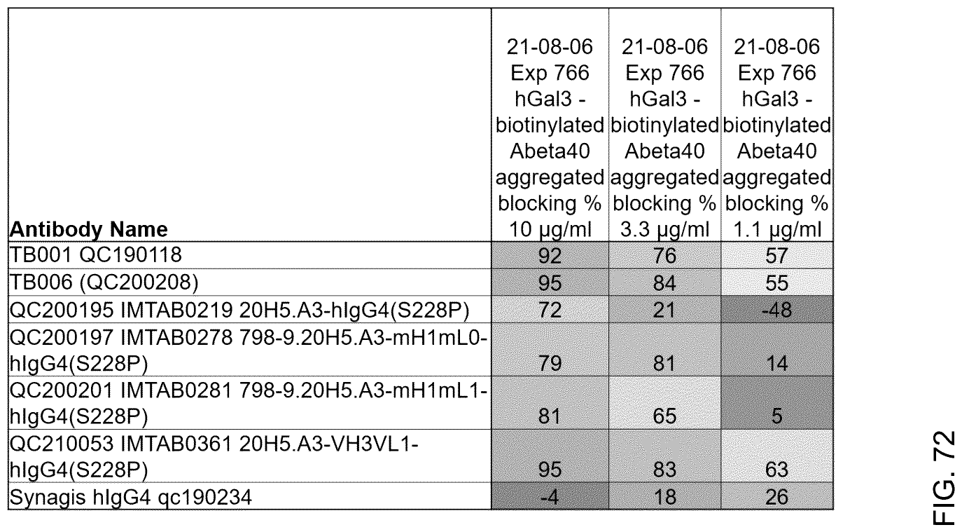

FIG. 72 is a table quantifying the effect of various anti-Gal2 antibodies at blocking hGal3 binding to Aβ40. As can be seen in FIG. 72, TB001 QC190118, TB006 (QC200208), QC200195 IMTAB0219 20H5.A3-hIgG4(S228P), QC200197 IMTAB0278 798-9.20H5.A3-mH1mL0-hIgG4(S228P), QC200201 IMTAB0281 798-9.20H5.A3-mH1mL1-hIgG4(S228P), and QC210053 IMTAB0361 20H5.A3-VH3VL1-hIgG4(S228P) demonstrate significant hGA13: Aβ40 blocking ability.

### Example 35: Gal3 Antibodies Block Gal3-Induced Aggregation of Insulin Aggregation

Insulin aggregation can lead to insulin-derived amyloidosis and diabetes. To test whether anti-Gal3 antibodies could be utilized to inhibit diabetes or insulin-derived amyloidosis, it was first established that Gal3 promotes insulin aggregation.

To establish that Gal-3 promotes insulin aggregation, insulin stocks (Sigma Cat #91077C Gal-3: QCB210021), were prepared at a stock concentration of 5.165 mg/mL. Insulin (200 ug/mL) with or without gal-3 (100, 200 ug/mL) was stirred in a 20% acetic acid solution, containing 100 mM NaCl at 50 degrees. Samples were dotted at 30 min, 1 h, 2 h, 3 h, 4 h, and the blot was probed with A11 antibody (Invitrogen Cat #AHB0052, 1:1000 dilution) overnight at 4 degrees. After incubating with respective secondary antibodies for 1 h, images were developed using Azure image developer.

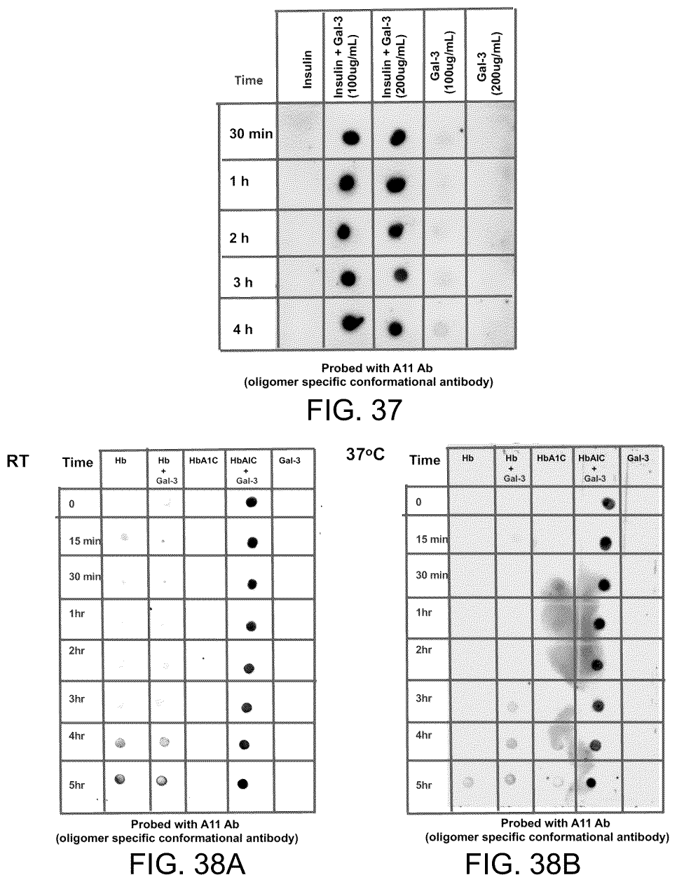

As can be seen in FIG. 37, Gal-3 promotes insulin aggregation.

FIG. 37 depicts 4 hour time course aggregation of insulin incubated at 50 C with 100 μg and 200 μg of Gal-3 probed with A11 antibody as detected by dot blot.

In an additional experiment to determine if galectin-3 oligomerizes with human insulin, insulin aggregates were prepared in 3 Eppendorf tubes labeled with the appropriate treatment (Insulin, Insulin+Gal 3 and Gal 3 alone). A corresponding dot blot was also prepared and labeled with a time course of 30 min, 1 hr, 2 hr and 4 hr. Insulin and Gal 3 were thawed at room temperature for 15-20 minutes. 1 mg of Insulin was weighed and reconstituted in 1 mL of buffer. (Insulin Stock Concentration was 1 mg/mL). Treatments having 200 ug/mL concentration (Insulin:200 ug/mL, Insulin and Gal 3: Both 200 ug/mL, and Gal 3 alone 200 ug/mL) were prepared in 500 μL of Buffer. The treatments were incubated at 50 degrees Celsius with continuous stirring. Samples were then dotted at 30 min, 1 h, 2 h, 3h, 4 h and the blot was probed with A11 antibody. After 4-hours, the Dot blot was rinsed once with 1×TBST and the membrane was blocked with 5% Milk in 1×TBST for 1 hour. After blocking, the membrane was washed 3 times (for 15 min each wash) with 1×TBST. The membrane was incubated with 1:1000 A11 antibody in 5% Milk for 1.5 hours at room temperature. Following incubation, the membrane was washed 3 times (for 15 min each wash) with 1×TBST and then incubated with 1:1000 Secondary Antibody Goat Anti-Rabbit IgG H&L (HRP) for 1 hour at Room Temperature. The membrane was then washed again 3 times (for 15 min each wash) with 1×TBST and developed. The dot blot was then prepared to be probed with 804 Anti Gal 3 antibody by stripping the membrane using stripping buffer for 15 minutes. The membrane was then washed 3 times (for 15 min each wash) with 1×TBST and blocked with 5% milk in TBST for 1 hr. Following blocking, the membrane was incubated with 1:1000 mouse Gal 3 804 Ab in 5% Milk in 1×TBST for overnight at 4 degrees. The membrane was then washed 3 times (for 15 min each wash) with 1×TBST and incubated the membrane with 1:1000 Secondary Antibody Goat anti mouse IgG (HRP) for 1 hr. at room temperature. Following incubation, the membrane was washed 3 times with 1×TBST and developed.

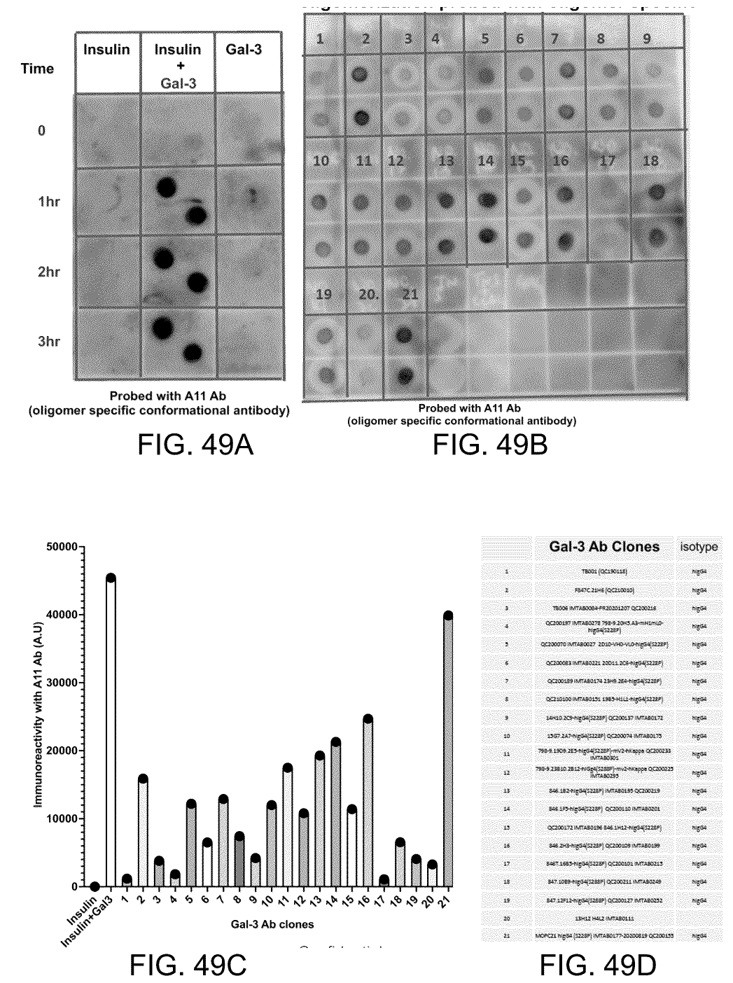

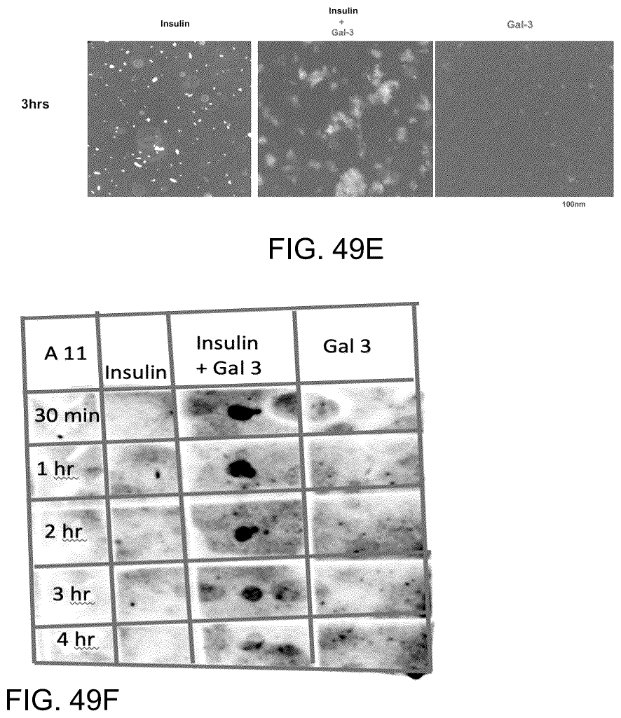

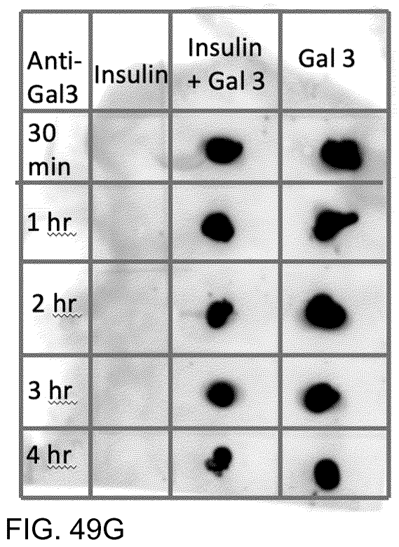

FIG. 49F is a dot blot depicting time course aggregation of insulin incubated with Gal3 and probed with A11 antibody. As can be seen in FIG. 49F, Gal3 promotes oligomerization of insulin after as little as 30 minutes of incubation. No aggregation was observed with insulin or galectin-3 alone.

FIG. 49G is a dot blot depicting time course aggregation of insulin incubated with Gal3 and probed with anti Gal-3 antibody 804. As can be seen in FIG. 49G, Gal3 promotes oligomerization of insulin after as little as 30 minutes of incubation.

To test whether anti-Gal3 antibodies could be utilized to inhibit proteopathies, it was first established using an ELISA that Gal3 is involved in the aggregation of insulin. Prior to the ELISA, insulin was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Insulin solution for binding was prepared by diluting biotinylated aggregated insulin in 2% BSA in PBST to a concentration of 10 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

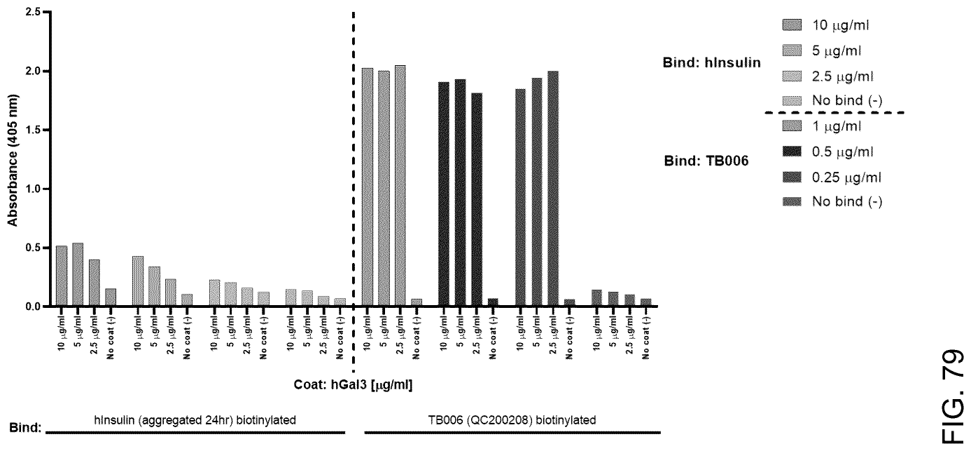

FIG. 79 depicts the results of the ELISA assay examining binding of hGal3 to aggregated insulin. As can be seen in FIG. 79, moderate binding of aggregated insulin to hGal3 was observed.

21 different Gal-3 antibody clones were then tested to determine if anti-Gal-3 antibodies block insulin aggregation. As can be seen in FIG. 49, all Gal-3 antibody clones displayed decreased insulin oligomerization as compared to insulin treated with Gal-3.

FIG. 49A-E depict embodiments of insulin oligomerization (incubated at 50 C) by Gal-3 and screening of different Gal-3 Ab clones for degrading insulin oligomerization probed with A11 Ab. FIG. 49A is a dot blot depicting a 3-hour time course of insulin oligomerization with and without Gal-3. FIG. 49B is a dot blot depicting screening of 21 different Gal-3 Ab clones for degrading insulin. FIG. 49C depicts quantification of inhibition of insulin oligomerization by 21 different Gal-3 Ab clones. FIG. 49D is a chart depicting the identity of each of the 21 different Gal-3 Ab clones screened. FIG. 49E depicts visualization of insulin aggregation with and without Gal-3 as detected using fluorescent microscopy.

### Example 36: Gal3 Promotes Calcitonin Aggregation

Calcitonin aggregation can lead to medullary carcinoma of the thyroid (MTC), Osteoporosis & Paget's Disease. To test whether anti-Gal3 antibodies could be utilized to inhibit diabetes or insulin-derived amyloidosis, it was first established that Gal3 promotes calcitonin aggregation.

FIG. 50 depicts a 48-hour time course of recombinant human calcitonin (hCT) aggregation when incubated with 100 μg of Gal-3 probed with A11 antibody as detected by dot blot.

Briefly, hCT was dissolved in 50 mM PBS buffer (pH 7.4) containing 100 mM NaCl to a final concentration of 25 μM. The solution was continuously stirred during the aggregation time course using a stir bar for 24 hrs at 37° C. The aggregation profile of hCT with out and with rhGal-3 was then determined by probing with oligomer specific antibody A11. After incubating with respective secondary antibodies for 1 h, images were developed using Azure image developer.

As can be seen in FIG. 50, Gal-3 promotes aggregation of calcitonin.

To test whether anti-Gal3 antibodies could be utilized to inhibit proteopathies, it was first established using an ELISA that Gal3 is involved in the aggregation of calcitonin. Prior to the ELISA, ANP was aggregated by multiple days (0, 1, 2 days) of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 10 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Calcitonin solution for binding was prepared by diluting biotinylated aggregated calcitonin in 2% BSA in PBST to a concentration of 10 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

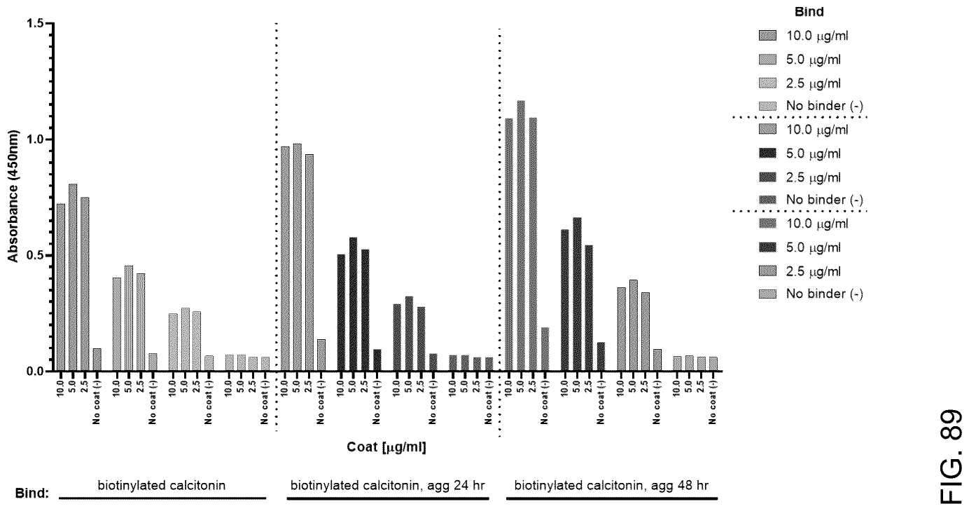

FIG. 89 depicts the results of the ELISA assay examining binding of hGal3 to various versions of calcitonin with hGal3. As can be seen in FIG. 89, calcitonin is a moderate binder of Gal3. Aggregation of calcitonin results in a minor increase in binding affinity.

### Example 37: Gal3 Promotes Phenylalanine Aggregation

Phenylalanine (Phe) aggregation can result in Phenylketonuria. To test whether anti-Gal3 antibodies could be utilized to inhibit phenylketonuria, Gal3 was tested for its ability to promote oligomerization of Phe.

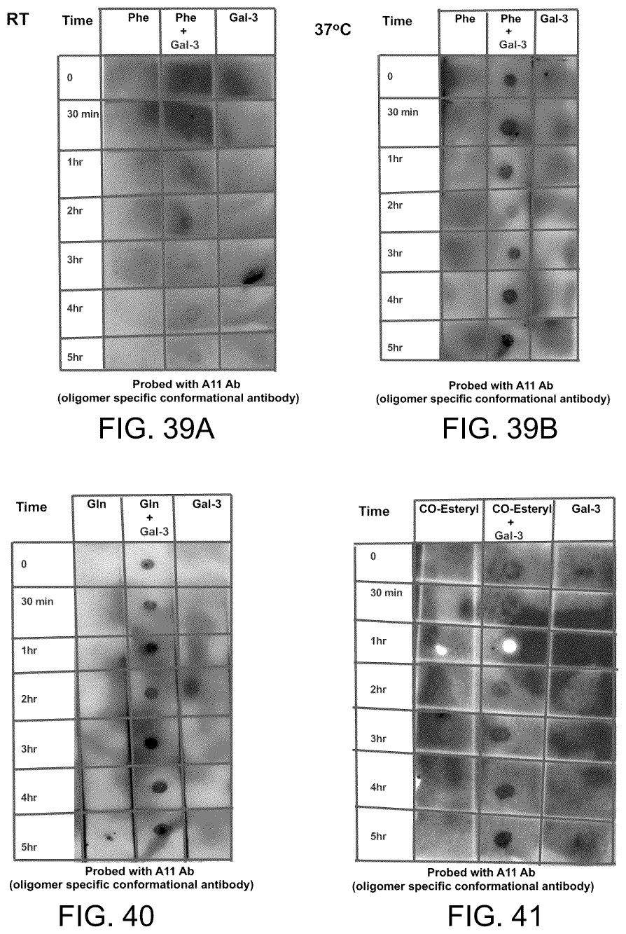

FIG. 39A-B depict 5 hour time course aggregation of phenylalanine (Phe) incubated at room temperature (RT) and 37 C with 100 μg of Gal-3 probed with A11 antibody as detected by dot blot.

Phenylalanine was diluted in 100 mM phosphate buffer saline, pH 7.4. The solution was continuously stirred during the aggregation time course using a stir bar for 24 hrs at 37° C. The aggregation profile of phenylamine with out and with rhGal-3 was determined by probing with A11. After incubating with respective secondary antibodies for 1 h, images were developed using Azure image developer.

As can be seen in FIG. 39A-B, Gal-3 promotes Phe aggregation.

### Example 38: Gal3 Promotes Glutamine Aggregation

Glutamine aggregation can result in Huntington disease. To test whether anti-Gal3 antibodies could be utilized to inhibit Huntington disease, Gal3 was tested for its ability to promote oligomerization of glutamine.

FIG. 40 depicts 5 hour time course aggregation of glutamine (GLN) incubated at room temperature (RT) with 100 μg of Gal-3 probed with A11 antibody as detected by dot blot.

Glutamine was diluted in 100 mM phosphate buffer saline, pH 7.4. The solution was continuously stirred during the aggregation time course using a stir bar for 24 hrs at 37° C. Aggregation profile of Glutamine with out and with rhGal-3 probed with A11 antibody. After incubating with respective secondary antibodies for 1 h, images were developed using Azure image developer.

As can be seen in FIG. 40, Gal-3 promotes glutamine aggregation.

### Example 39: Gal3 Promotes Myostatin Aggregation

Myostatin aggregation can result in idiopathic inflammatory myopathies (JIM). To test whether anti-Gal3 antibodies could be utilized to inhibit JIM, Gal3 was tested for its ability to promote oligomerization of glutamine.

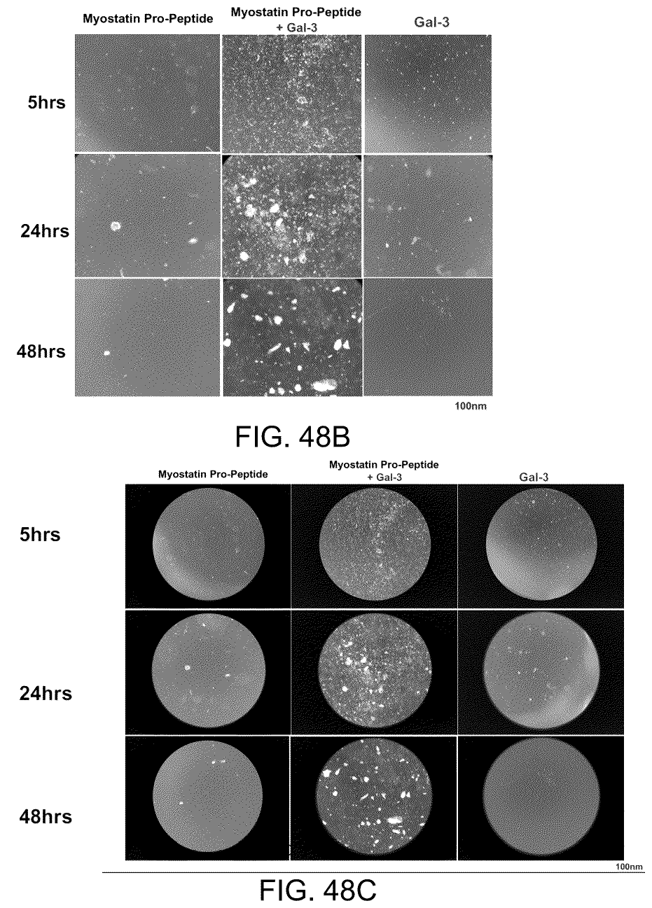

FIG. 48A-C depict 24 hour time course aggregation of myostatin pro-peptide incubated with 100 μg of Gal-3 probed with A11 antibody. FIG. 48A depicts 24 hour time course aggregation of Myostatin pro-peptide incubated with 100 μg of Gal-3 probed with A11 antibody as detected by dot blot. FIG. 48B-C depict visualization of myostatin pro-peptide aggregation at 5, 24, and 48 hours with and without Gal-3 as detected using fluorescent microscopy.

Myostatin was diluted in 100 mM phosphate buffer saline, pH 7.4. The solution was continuously stirred during the aggregation time course using a stir bar for 24 hrs at RT. Aggregation profile of Myostatin with out and with rhGal-3 probed with A11. After incubating with respective secondary antibodies for 1 h, images were developed using Azure image developer.

As can be seen in FIG. 48, Gal-3 promotes aggregation of myostatin.

### Example 40: Gal3 Promotes Lysozyme Aggregation

Lysozyme aggregation can result in idiopathic inflammatory myopathies (IIM). To test whether anti-Gal3 antibodies could be utilized to inhibit IIM, Gal3 was tested for its ability to promote oligomerization of lysozyme.

FIG. 36 depicts 24 hour time course aggregation of lysozyme incubated with 100 μg of Gal-3 probed with A11 antibody as detected by dot blot.

Lysozyme: Sigma Cat #L1667 Gal-3: QCB210021, stock concentration 5.165 mg/mL. Lysozyme (500 ug/mL) with or without gal-3 (100 ug/mL) were stirred in a 50 mM sodium phosphate buffer (pH 6.0) containing 2 M guanidine hydrochloride buffer at R.T. Samples were dotted at 0 min, 15 min, 30 min, 1, 2, 3, 4, 5 and 24 hr. Blot was probed with A11 antibody (Invitrogen Cat #AHB0052, 1:1000 dilution) overnight at 4 degrees. After incubating with respective secondary antibodies for 1 h, images were developed using Azure image developer.

As can be seen in FIG. 36, Gal-3 promotes lysozyme aggregation.

### Example 41: Gal3 Promotes Native Haemoglobin (Hb) and Glycosylated Haemoglobin (HbAIC) Aggregation

Gal3 was tested for its ability to promote oligomerization of native haemoglobin (Hb) and glycosylated haemoglobin (HbAIC).

FIG. 38A-B depicts 5 hour time course aggregation of native haemoglobin (Hb) and glycosylated haemoglobin (HbAIC) incubated at room temperature (RT) and 37 C with 100 μg of Gal-3 probed with A11 antibody as detected by dot blot. FIG. 38A depicts 5 hour time course aggregation of native haemoglobin (Hb) incubated at room temperature (RT) and 37 C with 100 μg of Gal-3 probed with A11 antibody as detected by dot blot. FIG. 38B depicts 5 hour time course aggregation of glycosylated haemoglobin (HbAIC) incubated at room temperature (RT) and 37 C with 100 μg of Gal-3 probed with A11 antibody as detected by dot blot.

Hb and HbA1c was dissolved in 20 mM Phosphate Buffer at pH 7.4. The solution was continuously stirred during the aggregation time course using a stir bar for 24 hrs at RT & 37° C. 2 μl of each sample was pipetted onto a Whatman nitrocellulose membrane at the appropriate time points (time 0-5 hr) for dot blot. The aggregation profile of Hb and HbA1C with and without rhGal-3 was determined by probing with antibody A11. After incubating with respective secondary antibodies for 1 h, images were developed using Azure image developer.

As can be seen in FIG. 38A-B, Galectin-3 intrinsically promotes the aggregation of HbA1c both at RT and 37° C., while as Hb was not aggregated with and without Galectin3.

### Example 42: Gal3 Promotes Light Chain Aggregation

Light chain aggregation can result in light chain (AL) amyloidosis. To test whether anti-Gal3 antibodies could be utilized to inhibit AL, Gal3 was tested for its ability to promote oligomerization of light chain.

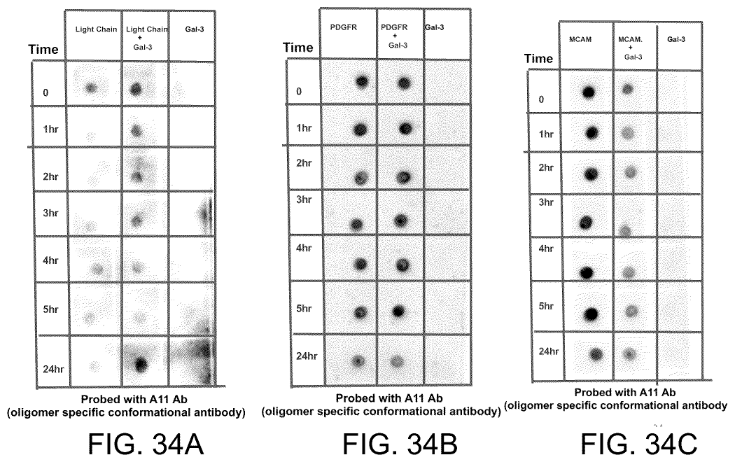

FIG. 34A-C depict time course aggregation of Light Chain, PDGFR, and MCAM, incubated with 100 μg of Gal-3 probed with A11 antibody as detected by dot blot. FIG. 34A depicts time course aggregation of Light Chain incubated with 100 μg of Gal-3 probed with A11 antibody as detected by dot blot. FIG. 34B depicts time course aggregation of PDGFR incubated with 100 μg of Gal-3 probed with A11 antibody as detected by dot blot. FIG. 34C depicts time course aggregation of MCAM incubated with 100 μg of Gal-3 probed with A11 antibody as detected by dot blot.

Light Chain (500 ug/mL) with or without gal-3 (100 ug/mL) was stirred in a 100 mM sodium phosphate buffer (pH 7.4) at R.T. Samples were dotted at spotted at d. The blot was then probed with A11 antibody (Invitrogen Cat #AHB0052, 1:1000 dilution) overnight at 4 degrees. After incubating with respective secondary antibodies for 1 h, images were developed using Azure image developer.

As can be seen in FIG. 34A-C, Gal-3 promotes aggregation of light chain.

### Example 43: Gal3 Promotes Cholesterol Aggregation

Cholesterol aggregation can result in atherosclerosis, cardiovascular disease and Alzheimer's disease. To test whether anti-Gal3 antibodies could be utilized to inhibit atherosclerosis, cardiovascular disease and Alzheimer's disease, Gal3 was tested for its ability to promote oligomerization of cholesterol.

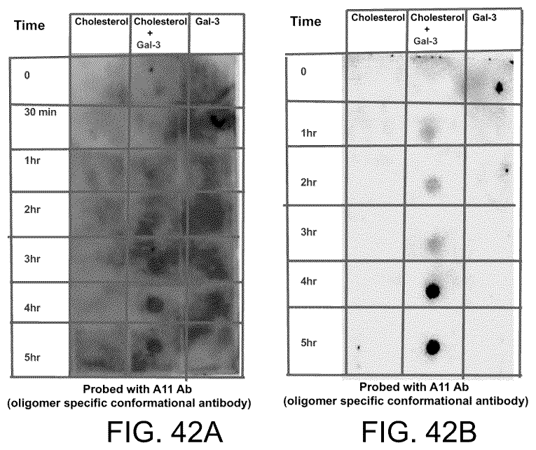

FIG. 42A-B depicts 5 hour time course aggregation of cholesterol incubated at room temperature (RT) with 100 μg of Gal-3 probed with antibody as detected by dot blot.

Cholesterol was dissolved in Chloroform and methanol in ratio of 2:1. Chloroform was evaporated and cholesterol was diluted in PBS, pH 7.4. The solution was continuously stirred during the aggregation time course using a stir bar for 24 hrs at 37° C. 2 μl of each sample was pipetted onto a Whatman nitrocellulose membrane at the appropriate time points (time 0-5 hr) for dot blot. Aggregation profile of Cholesterol with and without rhGal-3 probed with antibody A11. After incubating with respective secondary antibodies for 1 h, images were developed using Azure image developer.

As can be seen in FIG. 42A-B, Gal-3 promotes aggregation of cholesterol.

To test whether anti-Gal3 antibodies could be utilized to inhibit proteopathies, it was first established using an ELISA that Gal3 is involved in the aggregation of cholesterol. Prior to the ELISA, human cholesterol (Thermo, C3045) was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Cholesterol solution for binding was prepared by diluting biotinylated aggregated cholesterol in 2% BSA in PBST to a concentration of 40 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

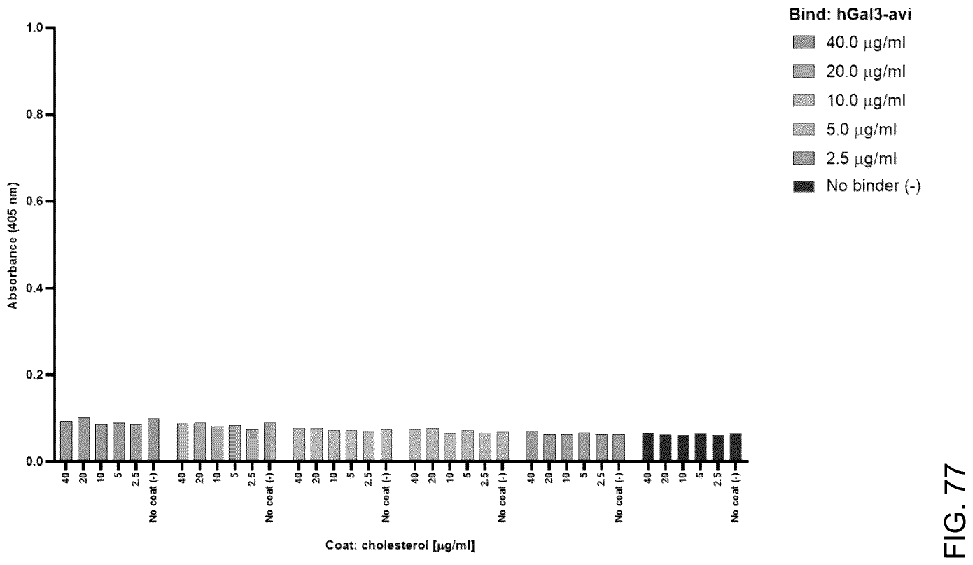

FIG. 77 depicts the results of the ELISA assay examining binding of hGal3 to aggregated cholesterol. As can be seen in FIG. 77, no binding of aggregated cholesterol was observed.

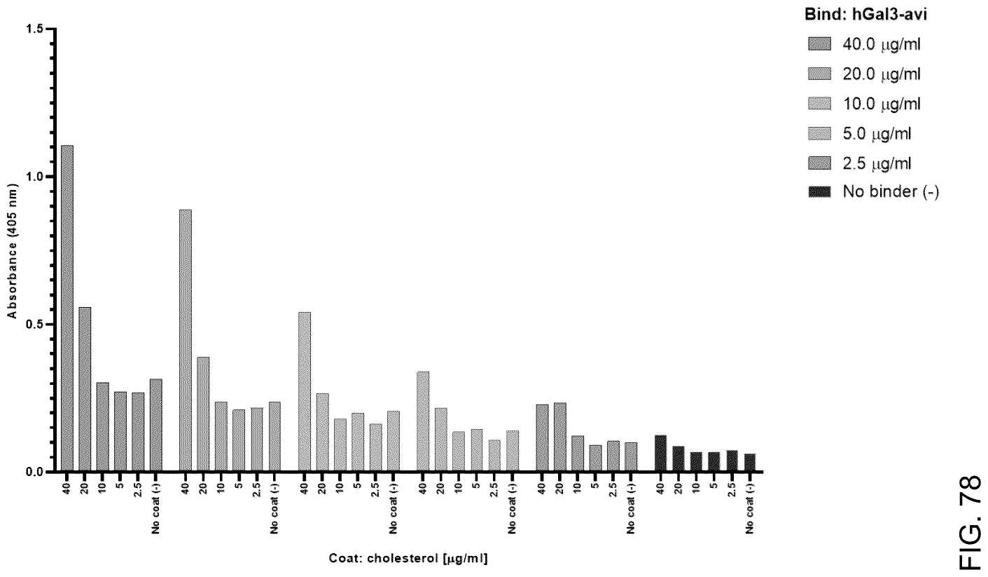

FIG. 78 depicts the results of the ELISA assay examining binding of hGal3 to 5 hour aggregated cholesterol. As can be seen in FIG. 77, moderate hGAL3 binding of aggregated cholesterol was observed.

### Example 44: Gal3 Promotes Cholesteryl Aggregation

Cholesteryl (Co-Esteryl) aggregation can result in atherosclerosis, cardiovascular disease and Alzheimer's disease. To test whether anti-Gal3 antibodies could be utilized to inhibit atherosclerosis, cardiovascular disease and Alzheimer's disease, Gal3 was tested for its ability to promote oligomerization of cholesteryl.

FIG. 41 depicts 5 hour time course aggregation of cholesteryl (Co-Esteryl) incubated at room temperature (RT) with 100 μg of Gal-3 probed with A11 antibody as detected by dot blot.

Cholesteryl was dissolved in Chloroform. Chloroform was evaporated and cholesteryl was diluted in PBS, pH 7.4. The solution was continuously stirred during the aggregation time course using a stir bar for 24 hrs at 37° C. 2 μl of each sample was pipetted onto a Whatman nitrocellulose membrane at the appropriate time points (time 0-5 hr) for dot blot. Aggregation profile of Cholesteryl with and without rhGal-3 probed with A11. After incubating with respective secondary antibodies for 1 h, images were developed using Azure image developer.

As can be seen in FIG. 41, Gal-3 promotes aggregation of cholesteryl.

### Example 45: Gal3 Promotes Atrial Natriuretic Peptide Aggregation

Atrial Natriuretic Peptide (ANP) aggregation can result in congestive heart failure (CHF) and cardiac amyloidosis. To test whether anti-Gal3 antibodies could be utilized to inhibit congestive heart failure (CHF) and cardiac amyloidosis, Gal3 was tested for its ability to promote oligomerization of ANP.

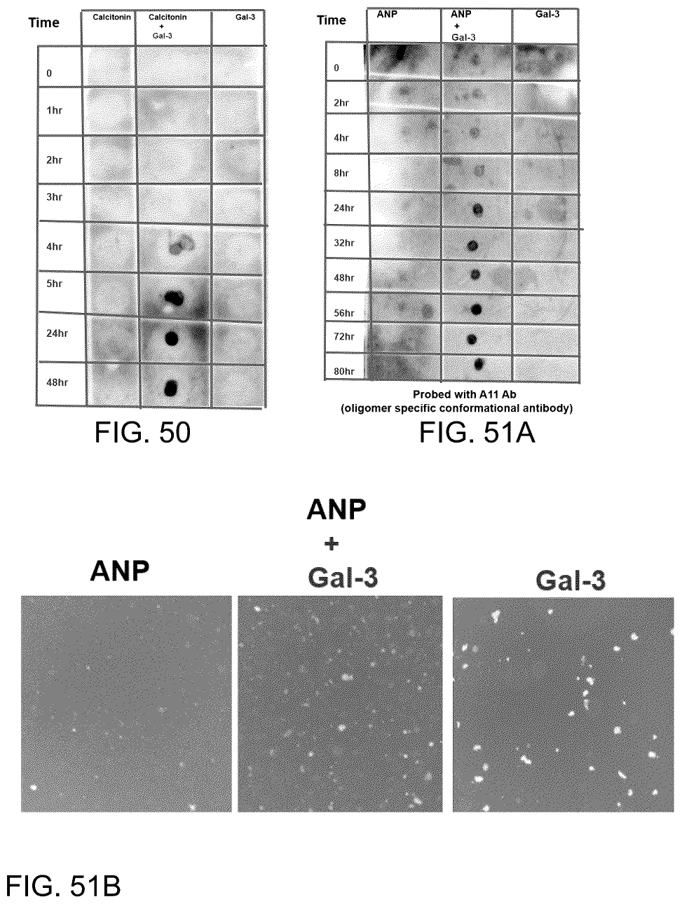

FIG. 51A-B depict Gal-3 promotion of Atrial Natriuretic Peptide (ANP) aggregation. FIG. 51A depicts embodiments of an 80-hour time course of ANP aggregation when incubated with 100 μg of Gal-3 probed with A11 antibody as detected by dot blot. FIG. 51B depicts embodiments of fluorescent microscopic visualization of ANP aggregation when incubated with 100 μg of Gal-3 probed with A11 antibody.

Three sets of proteins with concentrations 100 ug/ml each, ANP or Gal3 alone or in combinations (ANP+Gal3) were incubated in distilled water pH 5.75 with continued stirring for the 0-48 hrs time points at RT. The oligomerization were assayed using dot blot analysis. 2 ul of protein aliquots from each time points for all three sets were added to nitrocellulose membranes. The identical membranes were blocked with 5% non-fat dry milk for 1 h, followed by overnight incubation with ANP, Gal3 and A11 antibodies. After incubating with respective secondary antibodies for 1 h, images were developed using Azure image developer.

As can be seen in FIG. 51, Gal-3 promotes aggregation of ANP.

Gal3 binding to aggregated or unaggregated ANP was also tested using an Elisa. Prior to the ELISA, ANP was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. ANP solution for binding was prepared by diluting biotinylated aggregated ANP in 2% BSA in PBST to a concentration of 10 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

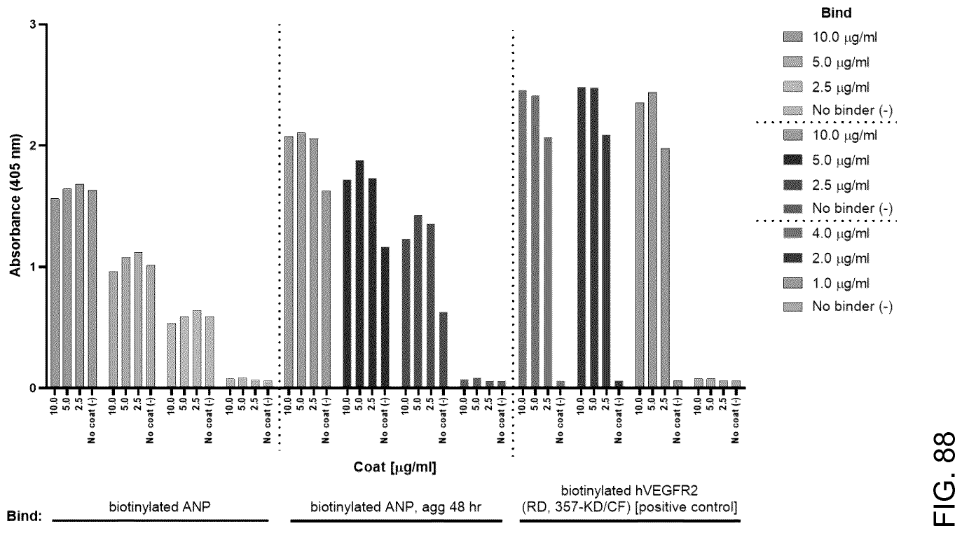

FIG. 88 depicts the results of the ELISA assay examining binding of hGal3 to aggregated and unaggregated ANP. As can be seen in FIG. 88, ANP is likely a weak binder or non-binder of Gal3 when un-aggregated. Aggregation of ANP causes a significant increase in ANP:Gal3 binding.

### Example 46: Gal3 Promotes Pro-B Type Natriuretic Peptide Aggregation

Pro-B type Natriuretic Peptide (BNP) aggregation can result in congestive heart failure (CHF) and cardiac amyloidosis. To test whether anti-Gal3 antibodies could be utilized to inhibit congestive heart failure (CHF) and cardiac amyloidosis, Gal3 was tested for its ability to promote oligomerization of BNP.

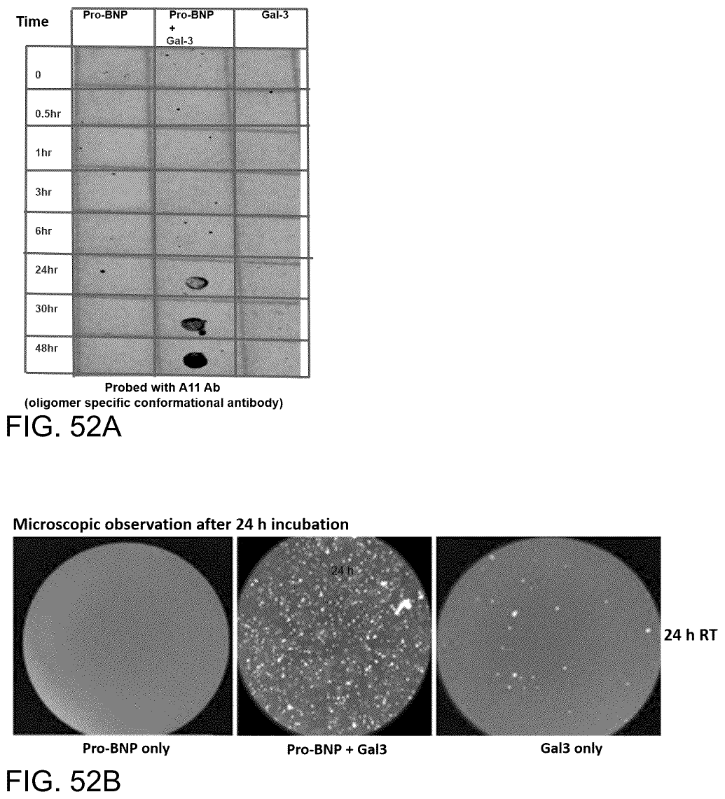

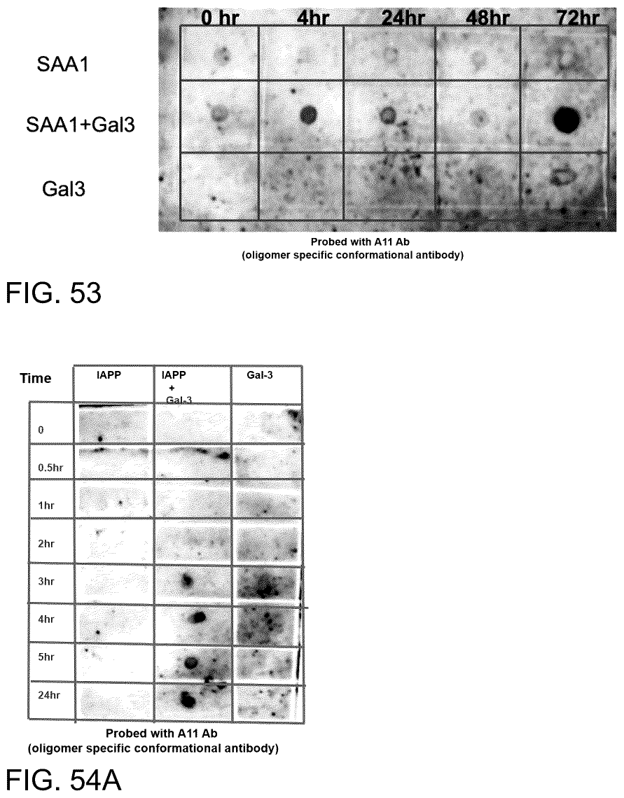

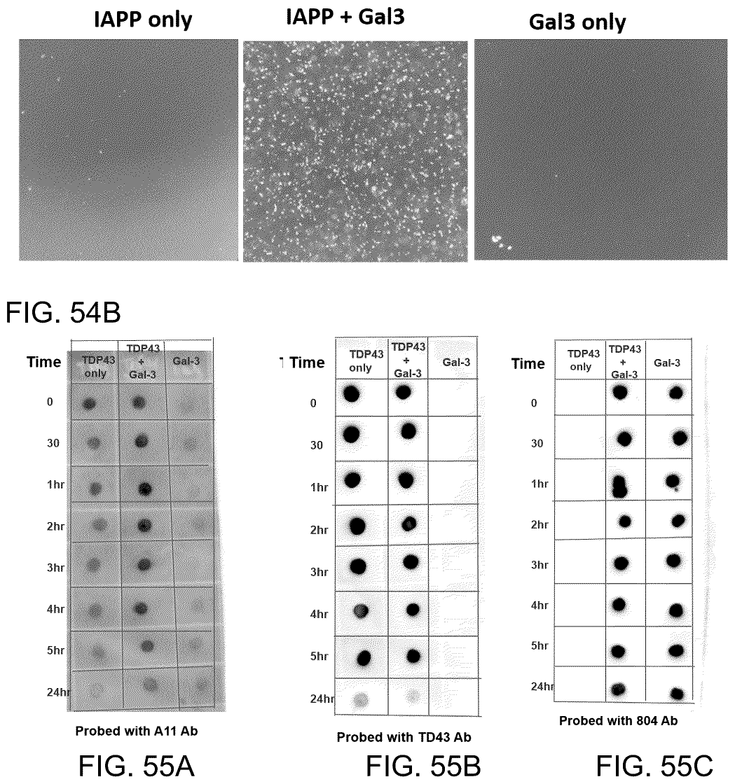

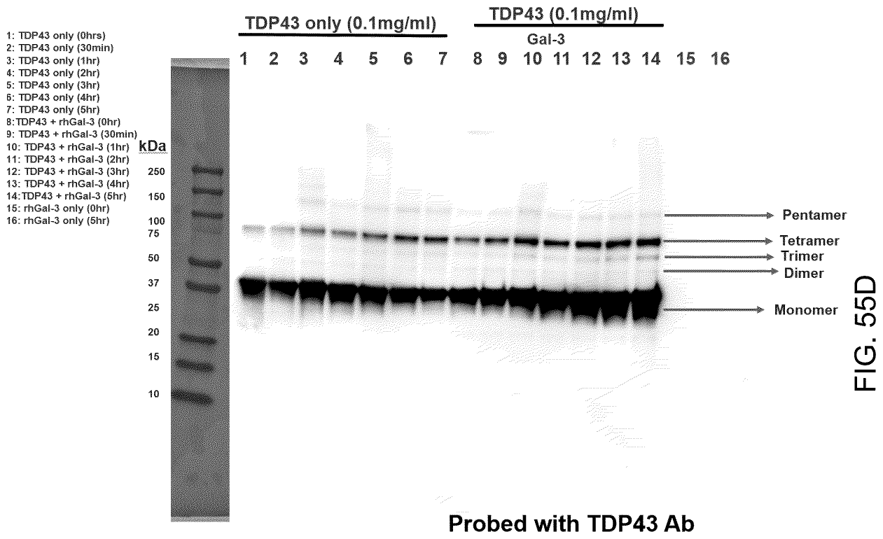

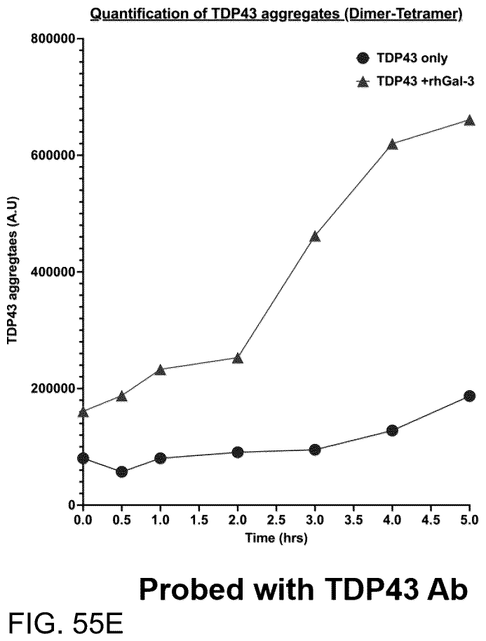

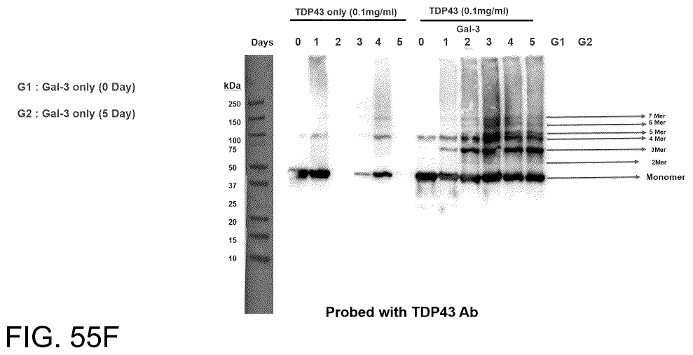

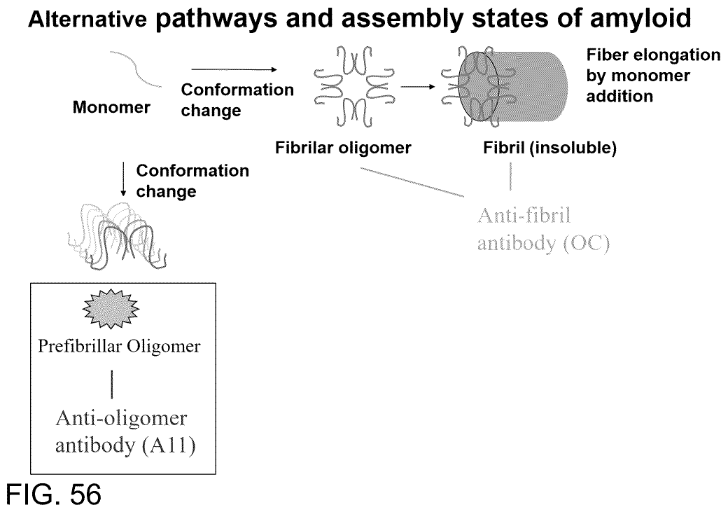

FIG. 52A-B depict Gal-3 promotion of Pro-B type Natriuretic Peptide (BNP) aggregation. FIG. 52A depicts embodiments of a 48-hour time course of BNP aggregation when incubated with 100 μg of Gal-3 probed with A11 antibody as detected by dot blot. FIG. 52B depicts embodiments of fluorescent microscopic visualization of BNP aggregation when incubated with 100 μg of Gal-3 probed with A11 antibody and incubated for 24-hours at room temperature.

Three sets of proteins with concentrations 100 ug/ml each, NT-Pro BNP or Gal3 alone or in combinations (NT-Pro BNP+Gal3) were incubated in distilled water pH 5.75 with continued stirring for the 0-48 hrs time points at RT. The Oligomerization were assayed using dot blot analysis. 2 ul of protein aliquots from each time points for all three sets were added to nitrocellulose membranes. The identical membranes were blocked with 5% non-fat dry milk for 1 h, followed by overnight incubation with BNP, Gal3 and A11 antibodies. After incubating with respective secondary antibodies for 1 h, images were developed using Azure image developer.

As can be seen in FIG. 52, Gal-3 promotes aggregation of BNP.

### Example 47: Gal3 Promotes Cystatin-C Aggregation

Cystatin-C aggregation can result in kidney disease, CAA, or Alzheimer's disease. To test whether anti-Gal3 antibodies could be utilized to inhibit kidney disease, CAA, or Alzheimer's disease, Gal3 was tested for its ability to promote oligomerization of cystatin-c.

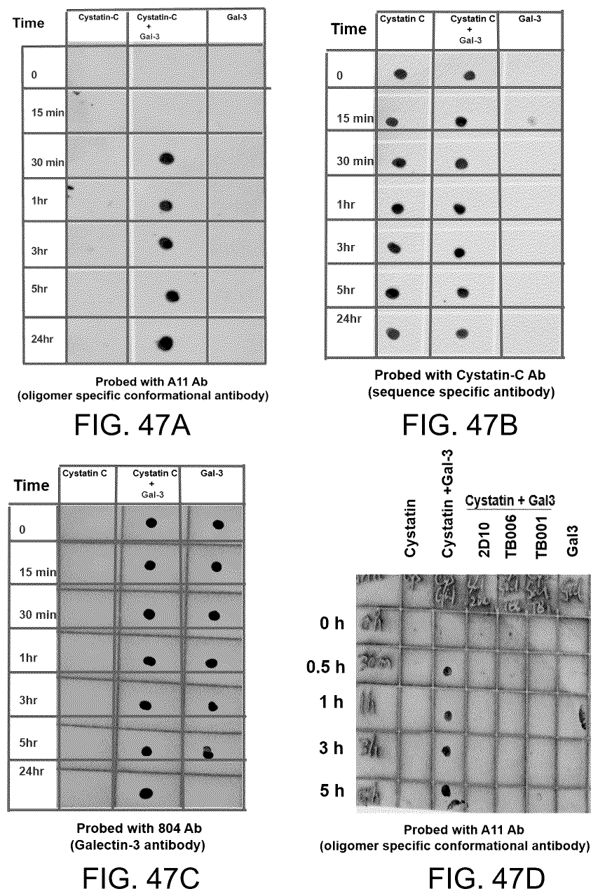

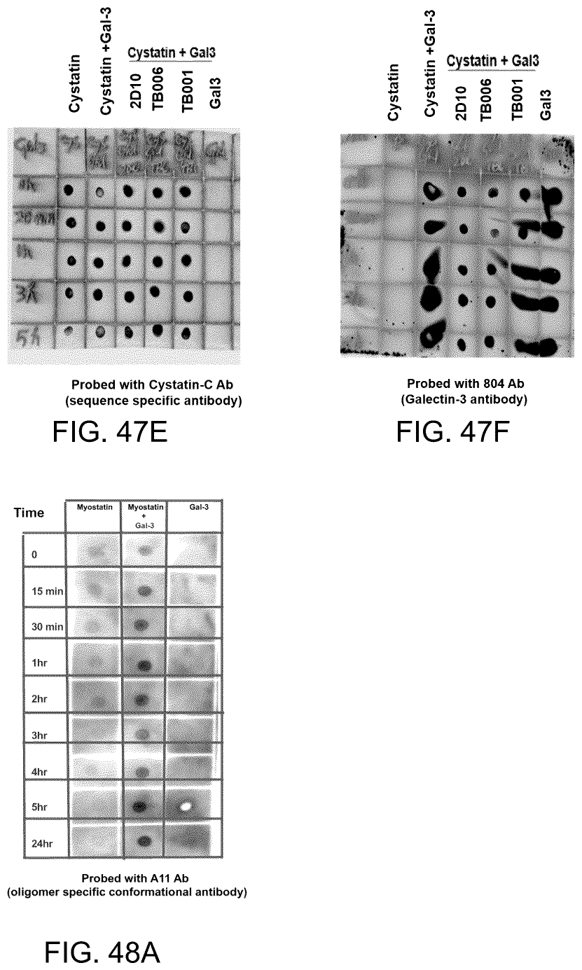

FIG. 47A-F depicts time course aggregation of Cystatin-C incubated with 100 μg of Gal-3 probed with A11 antibody as detected by dot blot. FIG. 47A depicts 24 hour time course aggregation of Cystatin-C incubated with 100 μg of Gal-3 probed with A11 antibody as detected by dot blot. FIG. 47B depicts 24 hour time course aggregation of Cystatin-C incubated with 100 μg of Gal-3 probed with Cystatin-C antibody as detected by dot blot. FIG. 47C depicts 24 hour time course aggregation of Cystatin-C incubated with 100 μg of Gal-3 probed with 804 antibody as detected by dot blot. FIG. 47D depicts 5 hour time course aggregation of Cystatin-C incubated with Gal-3 probed with A11 antibody as detected by dot blot. FIG. 47E depicts 5 hour time course aggregation of Cystatin-C incubated with Gal-3 probed with cystatin-C antibody as detected by dot blot. FIG. 47F depicts 5 hour time course aggregation of Cystatin-C incubated with Gal-3 probed with 804 antibody as detected by dot blot.

Three sets of proteins, 100 μg/ml each of Human Cystatin C, Gal3 alone or in combinations (Cystatin C+Gal3), were incubated in phosphate buffer pH 7.4 with continued stirring for the 0-24 hrs time points at 370 C. Oligomerization were assayed using dot blot analysis. 2 ul of protein aliquots from each time points for all three sets were added to nitrocellulose membranes. The identical membranes were blocked with 5% non-fat dry milk for 1 h, followed by overnight incubation with Cystatin C, Gal3 and A11 antibodies. After incubating with respective secondary antibodies for 1 h, images were developed using Azure image developer.

For Gal3 antibodies, two-fold concentration of 2D10, TB006, and TB001 were added to respective reaction tubes containing Gal3 and Cystatin C protein (100 ug/ml), and incubation with continued stirring was done as before.

As can be seen in FIG. 47A-F, Gal-3 promotes aggregation of cystatin-c.

### Example 48: Gal3 Promotes Fibrin Aggregation

Fibrin aggregation can result in cerebrovascular damage, stroke, CAA, or Alzheimer's disease. To test whether anti-Gal3 antibodies could be utilized cerebrovascular damage, stroke, CAA, or Alzheimer's disease, Gal3 was tested for its ability to promote oligomerization of fibrin.

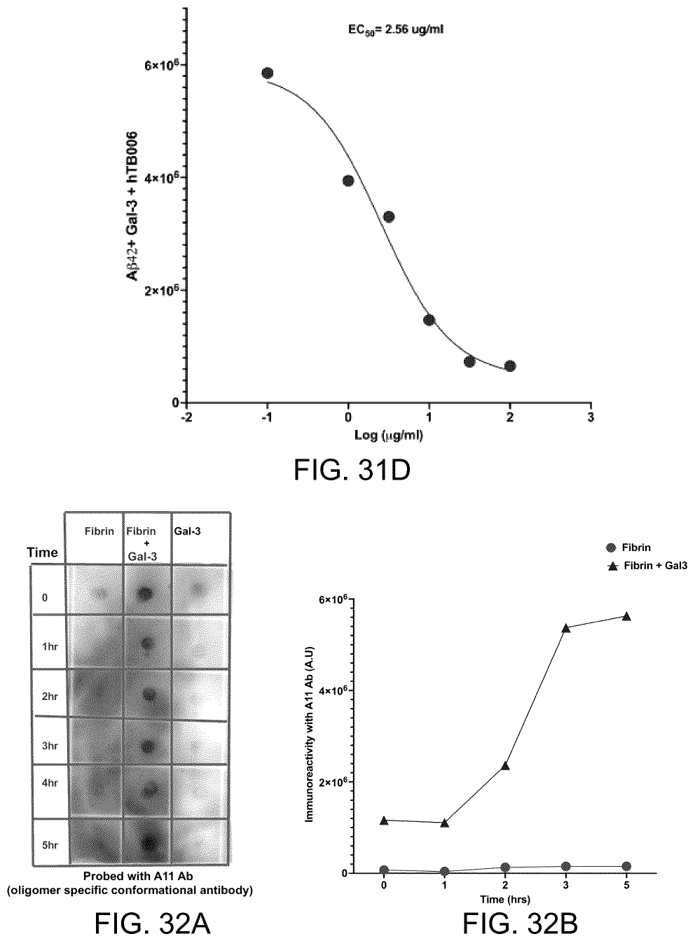

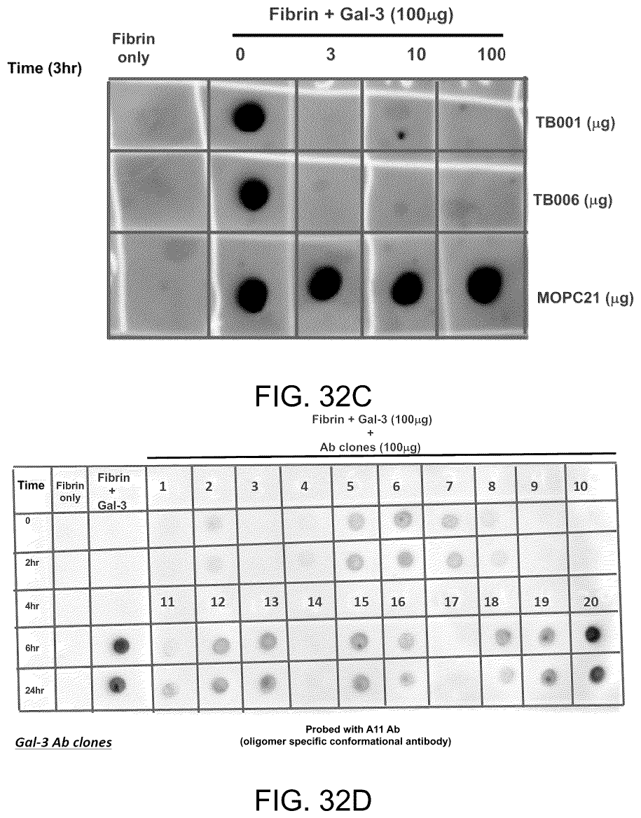

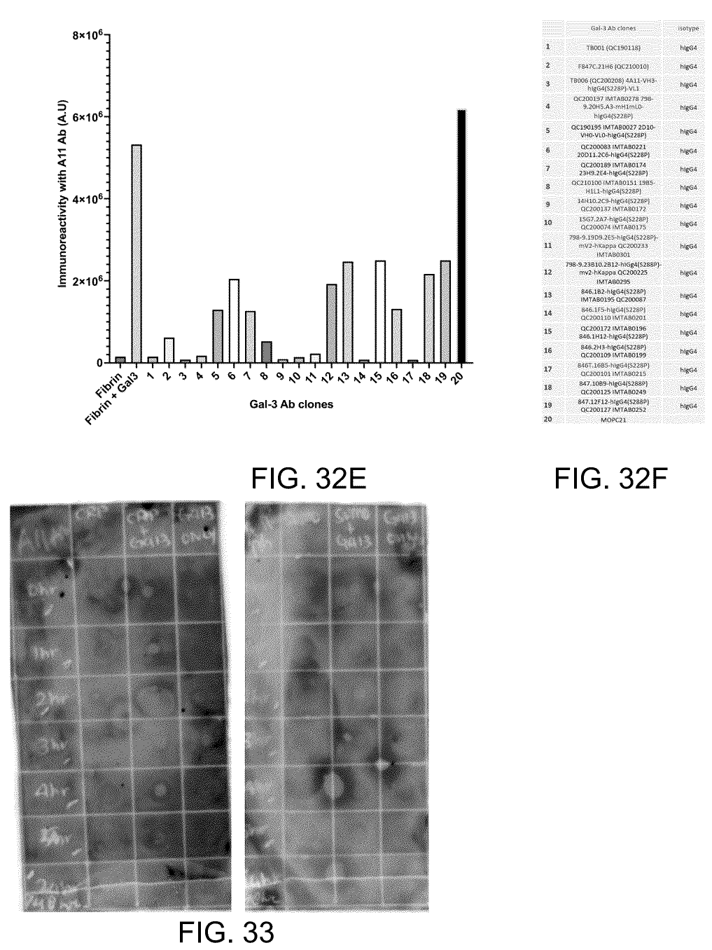

FIG. 32A-F depict time course aggregation of Fibrin incubated with 100 μg of Gal-3 probed with A11 as detected by dot blot. FIG. 32A depicts time course aggregation of Fibrin incubated with 100 μg of Gal-3 probed with A11 as detected by dot blot. FIG. 32B depicts quantification of Gal-3 intrinsic promotion of Fibrin oligomerization. FIG. 32C depicts degradation of toxic Fibrin oligomers by hTB001 and hTB006 as detected by dot blot. FIG. 32D depicts screening of different Gal-3 antibody clones on Fibrin oligomerization probed with A11 antibody. FIG. 32E depicts quantification of different Gal-3 antibody clones on Fibrin oligomerization probed with A11 antibody. FIG. 32F is a table depicting the identity and isotype of Gal-3 antibody clones screened.

1 mg of fibrin peptide was resuspended in 90 μl of 100 mM NaOH and incubated for 10 minutes. This solution was then diluted to final concentration 0.1 mg/ml by adding 10 mM sodium phosphate buffer, pH 7.4. Gal-3 and/or antibodies were added to test their effects. The solution was continuously stirred using a stir bar while incubating at room temperature. At various time points after mixing, samples were taken and probed on a dot blot to detect formation of oligomers using A11 antibody. After incubating with respective secondary antibodies for 1 h, images were developed using Azure image developer.

As can be seen in FIG. 32, Gal-3 promotes aggregation of fibrin.

Gal3 binding to fibrin was also tested using an Elisa. Prior to the ELISA, human fibrin was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. Fibrin solution for binding was prepared by diluting biotinylated aggregated fibrin in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

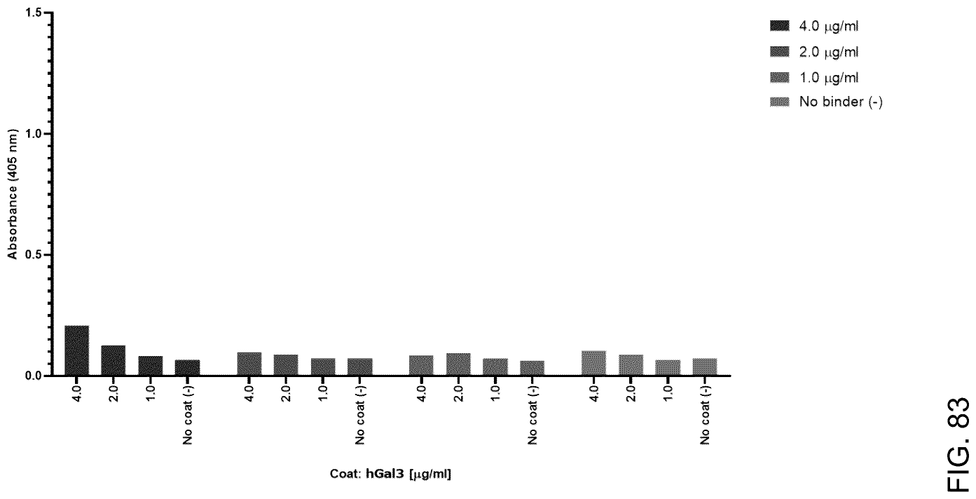

FIG. 83 depicts the results of the ELISA assay examining binding of hGal3 to aggregated NFL. As can be seen in FIG. 83, weak binding of hGal3 to aggregated NFL was observed.

### Example 49: Gal3 Promotes Aggregation of Complement Protein C3

Oligomerization of complement protein C3 can lead to disruption of the innate immune system. To test whether anti-Gal3 antibodies could be utilized to inhibit innate immune system disruption, it was first established that Gal3 promotes C3 aggregation.

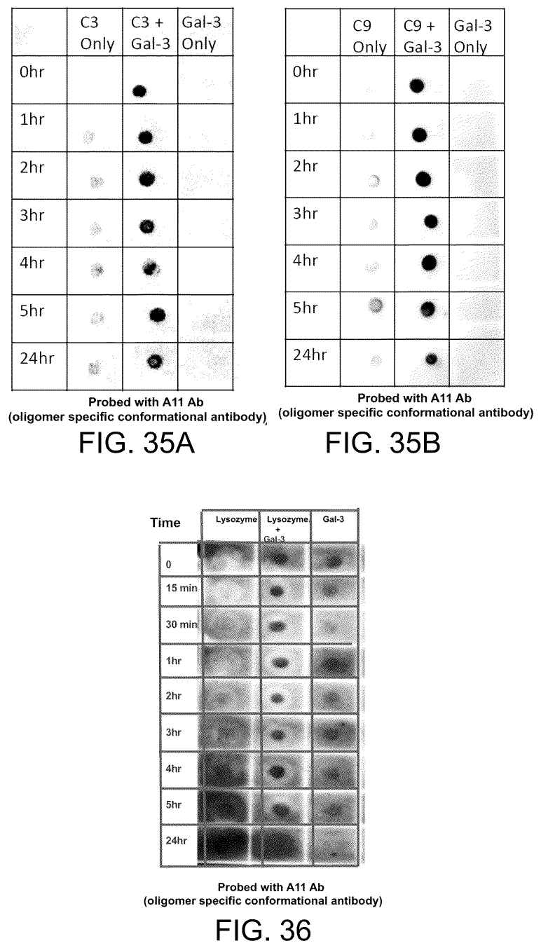

FIG. 35A-B depict 24 hour time course aggregation of complement proteins (C3 & C9) incubated with 100 μg of Gal-3 probed with A11 antibody as detected by dot blot. FIG. 35A depicts 24 hour time course aggregation of complement proteins C3 incubated with 100 μg of Gal-3 probed with A11 antibody as detected by dot blot. FIG. 35B depicts 24 hour time course aggregation of complement protein C9 incubated with 100 μg of Gal-3 probed with A11 antibody as detected by dot blot.

To determine whether oligomerized C3 binds hGal3, Gal3 binding to oligomerized C3 was tested using a dot blot. 1 mg of lyophilized C3 peptide was resuspended in 90 μl of 100 mM NaOH and incubated for 10 minutes. The solution was then diluted to final concentration 0.1 mg/ml by adding 100 mM sodium phosphate buffer, pH 7.4. Synthetic C3 and peptide were then mixed with rhGal-3 protein, and incubated for 1 hr at RT. After 1 hour, different concentrations of hTB001 and hTB006Ab were added and the solution was incubated for 5 hours at RT. 2 μl of each sample was pipetted onto a Whatman nitrocellulose membrane at the appropriate time points (time 0-5 hr) for a dot blot and probed with antibody A11.

As can be seen in FIG. 35A-B, rhGal-3 induces oligomerization of C3.

To determine whether oligomerized C3 binds hGal3, Gal3 binding to oligomerized C3 was also tested using an Elisa. Prior to the ELISA, human C3 was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. C3 solution for binding was prepared by diluting biotinylated aggregated C3 in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

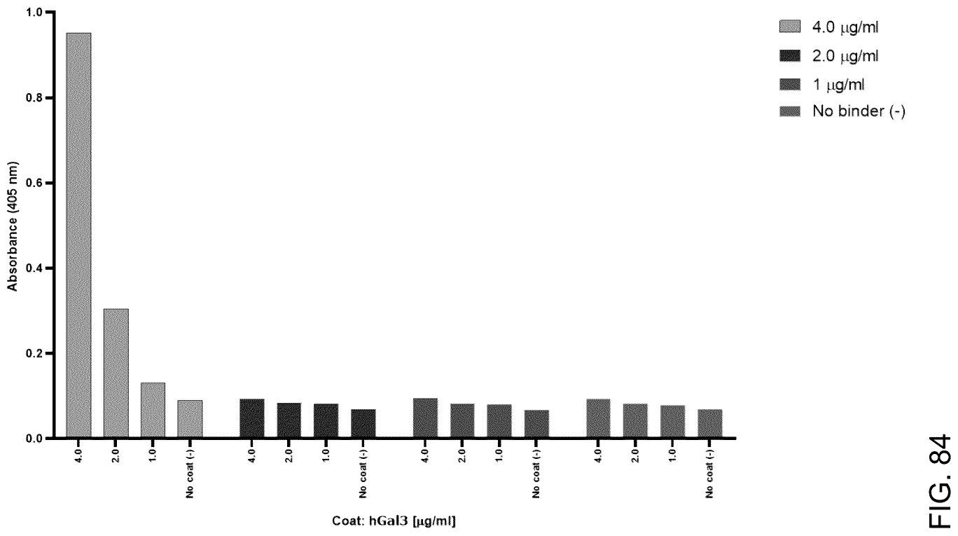

FIG. 84 depicts the results of the ELISA assay examining binding of hGal3 to aggregated C3. As can be seen in FIG. 84, hGal3 demonstrates moderate binding to C3 even at diminishing concentrations of Aβ40 or hGal3.

An ELISA was also used to determine the IC50s of various antibodies against galectin-3:: aggregated Complement C3. Prior to the ELISA, complement C3 (MyBioSource) was aggregated by 48 hours of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Human Galectin-3 (Gal3) protein (TrueBinding, QCB210019) was diluted in PBS to a concentration of 4 μg/ml and coated a 96-well ELISA plate by applying 35 μl to each well. After incubating the plate at 4° C. O/N, the plate was washed three times with 300 μl PBST. The plate was then blocked for an hour with 150 μl of 2% BSA in PBST at RT with gentle rocking. Thereafter, the 2% BSA in PBST was discarded and 30 μl of TB001 (TrueBinding, QC190118), TB006 (TrueBinding, QC200208), MOPC21 (TrueBinding, QC200153) (3-fold serial dilutions beginning at 30 μg/ml) in 2% BSA in PBST was added to the wells, immediately followed by the addition of 30 μl of biotinylated aggregated Complement C3 diluted to 4 μg/ml in 2% BSA in PBST. The plate was incubated for an hour at RT with gentle rocking and then washed three times with 300 μl PBST. Thereafter, Avidin HRP (Biolegend, 405103) was diluted in 2% BSA in PBST (1:2000 dilution) and 25 μl added to the wells. The plate was incubated at RT for an hour with gentle rocking and washed three times with 300 μl PBST. 50 μl of ABTS (Life Technologies, 00-2024) was added to each well to develop, then the plate was read using a plate reader at an absorbance of 405 nm. IC50 values were generated using GraphPad Prism 8.0 software (GraphPad Software Inc.), following the manufacturer's instructions.

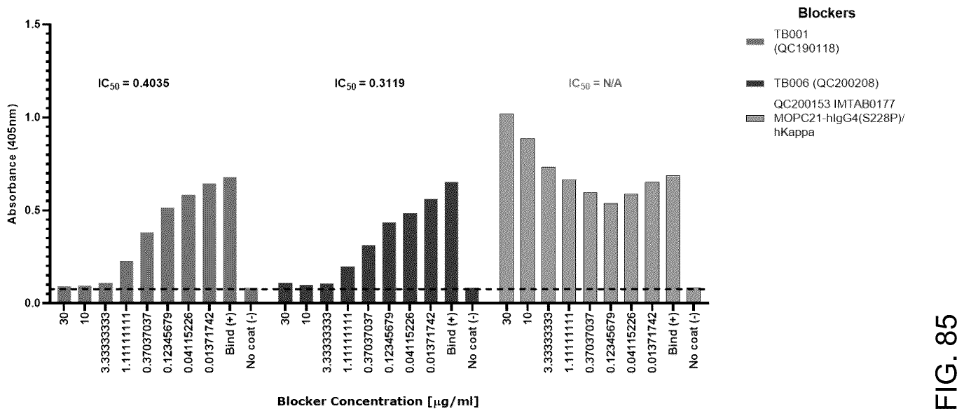

FIG. 85 depicts the results of the ELISA assay determining the IC50s of anti-hGal3 antibodies against hGal3:C3. As can be seen in FIG. 85, anti-hGal3 antibodies display a range of IC50s.

### Example 50: Gal3 Promotes Aggregation of Complement Protein C9

Oligomerization of complement protein C9 can lead to disruption of the innate immune system. To test whether anti-Gal3 antibodies could be utilized to inhibit innate immune system disruption, it was first established that Gal3 promotes C3 aggregation.

To determine whether oligomerized C9 binds hGal3, Gal3 binding to oligomerized C3 was tested using a dot blot. 1 mg of lyophilized C9 peptide was resuspended in 90 μl of 100 mM NaOH and incubated for 10 minutes. The solution was then diluted to final concentration 0.1 mg/ml by adding 100 mM sodium phosphate buffer, pH 7.4. Synthetic C9 and peptide were then mixed with rhGal-3 protein, and incubated for 1 hr at RT. After 1 hour, different concentrations of hTB001 and hTB006Ab were added and the solution was incubated for 5 hours at RT. 2 μl of each sample was pipetted onto a Whatman nitrocellulose membrane at the appropriate time points (time 0-5 hr) for a dot blot and probed with antibody A11.

As can be seen in FIG. 35B, rhGal-3 induces oligomerization of C9.

To determine whether oligomerized C9 binds hGal3, Gal3 binding to oligomerized C9 was also tested using an Elisa. Prior to the ELISA, human C9 was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

To start the ELISA, coating solutions were prepared by diluting galectin-3 protein (TrueBinding, QCB210019) (TrueBinding, QCB200377) (Biolegend, 599806) in separate volumes of PBS to a concentration of 4 μg/ml. Coating solutions were applied to a 96-well ELISA plate (Thermo Scientific, 44-2401-21) by adding 80 μl per well along the top row of the plate, four wells per protein, then serially diluting two-fold downwards in PBS. After incubating the plate at 4° C. O/N, the plate was washed with 300 μl PBST three times, followed by a blocking step in which 150 μl of 2% BSA in PBST was added to every well and incubated for an hour at RT with gentle rocking. The existing blocking solution was then discarded from the plate. C9 solution for binding was prepared by diluting biotinylated aggregated C9 in 2% BSA in PBST to a concentration of 4 μg/ml, then applied to the plate by adding 60 μl per well column-wise for three columns, one column for each galectin-3, then serially diluted two-fold length-wise rightwards in 2% BSA in PBST. The plate was incubated for an hour at RT with gentle rocking and then washed with 300 μl PBST three times. Afterward, Avidin-HRP (Biolegend, 405103) was diluted to 1:2000 in 2% BSA in PBST, then 25 μl was added to all the wells. The plate was incubated for an hour at RT with gentle rocking, then washed with 300 μl PBST three times. To develop the plate, 50 μl of ABTS (Life Technologies, 00-2024) substrate was added to each well, incubated until a sufficient signal was reached, then stopped with 25 μl of 1N HCl per well. The plate was read in a plate reader at an absorbance of 405 nm. Data was graphed using GraphPad Prism 8.0 software (GraphPad Software Inc).

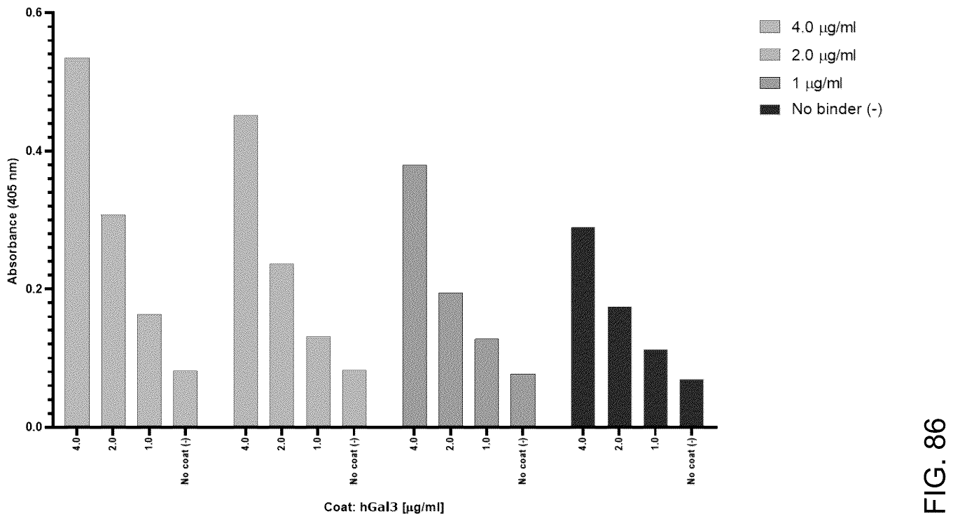

FIG. 86 depicts the results of the ELISA assay examining binding of hGal3 to aggregated C9.As can be seen in FIG. 86, hGal3 demonstrates weak binding to C9.

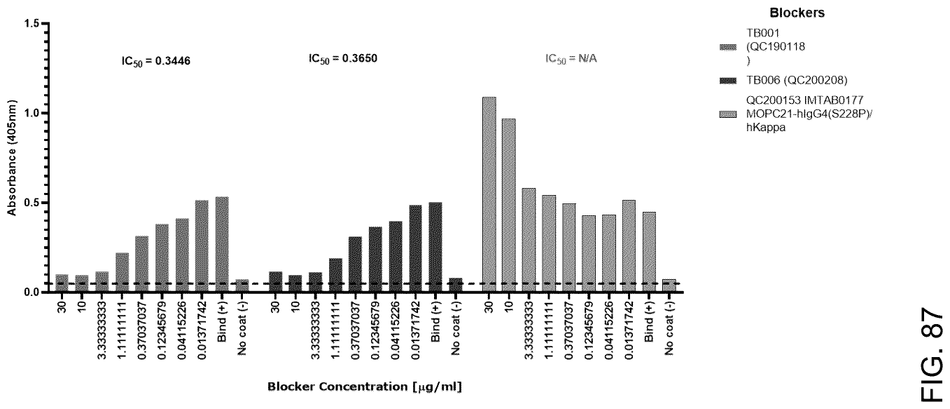

FIG. 87 depicts the results of the ELISA assay determining the IC50s of anti-hGal3 antibodies against hGal3:C9.

As can be seen in FIG. 87, anti-hGal3 antibodies display a range of IC50s.

### Example 51: Gal3 Promotes Aggregation of hTGFβR2

An ELISA was used to evaluate blocking efficacies of various anti-hGal3 antibodies against hGal3::hTGFβR2. Prior to the ELISA, hTGFβR2 was aggregated by 48 hours of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Prior to the ELISA, hTGFβR2 was aggregated by multiple days of stirring, biotinylated using EZ Link Sulfo-NHS-LC-Biotin (ThermoFisher Scientific, A39257), and desalted with a Zeba Spin Desalting Column (ThermoFisher Scientific, 89882) following the manufacturers' instructions.

Human Galectin-3 (Gal3) protein (TrueBinding, QCB200377) was diluted in PBS to a concentration of 4 μg/ml and coated a 96-well ELISA plate by applying 35 μl to each well. After incubating the plate at 4° C. O/N, the plate was washed three times with 300 μl PBST. The plate was then blocked for an hour with 150 μl of 2% BSA in PBST at RT with gentle rocking. Thereafter, the 2% BSA in PBST was discarded and 30 μl of TB001 (TrueBinding, QC190118), TB006 (TrueBinding, QC200208), several 20H5 (TrueBinding, various QC #), or Synagis hIgG4 Isotype (TrueBinding, QC190234) (3-fold serial dilutions beginning at 10 μg/ml) in 2% BSA in PBST was added to the wells, immediately followed by the addition of 30 μl of 1 μg/ml of biotinylated hTGFβR2 in 2% BSA in PBST. The plate was incubated for an hour at RT with gentle rocking and then washed three times with 300 μl PBST. Thereafter, Avidin HRP (Biolegend, 405103) was diluted in 2% BSA in PBST (1:2000 dilution) and 25 μl was added to the wells. The plate was incubated at RT for an hour with gentle rocking and washed three times with 300 μl PBST. 50 μl of ABTS (Life Technologies, 00-2024) was added to each well to develop, then the plate was read using a plate reader at an absorbance of 405 nm. Data was graphed using GraphPad Prism 8.0 software (GraphPad Software Inc).

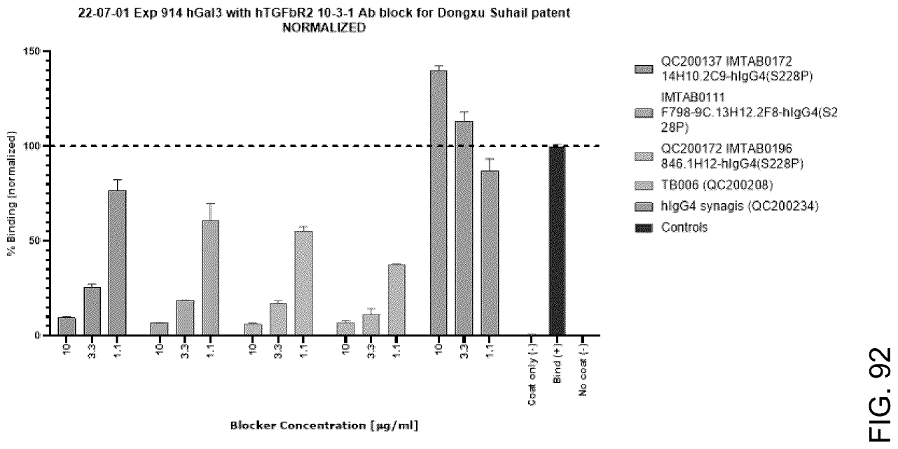

FIG. 92 depicts the results of the blocking efficacy determination as measured by Elisa.

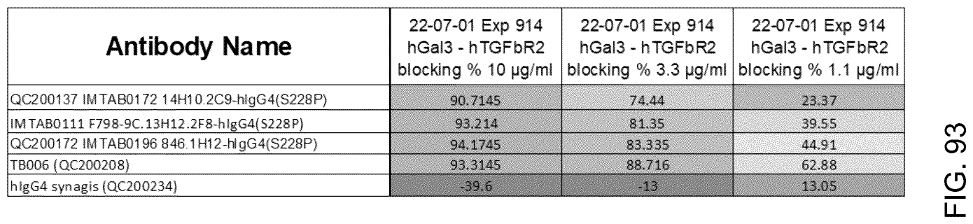

FIG. 93 is a table depicting the blocking efficacy of QC200137 IMTAB0172 14H10.2C9-hIgG4(S228P), IMTAB0111 F798-9C.13H12.2F8-hIgG4(S228P), QC200172 IMTAB0196 846.1H12-hIgG4(S228P), TB006 (QC200208), hIgG4 synagis (QC200234)(negative control).

As can be seen in FIG. 92-93, QC200137 IMTAB0172 14H10.2C9-hIgG4(S228P), IMTAB0111 F798-9C.13H12.2F8-hIgG4(S228P), QC200172 IMTAB0196 846.1H12-hIgG4(S228P), TB006 (QC200208) each able to block hGal3::hTGFbR2 with greater than 90% efficacy.

### Example 52: Comparative Effect of Small Molecular Gal-3 Inhibitor TD139 and TB006 on Degradation of Aβ42 Oligomers

TD139 and TB006 were tested for their relative ability to promote degradation of Aβ oligomers.

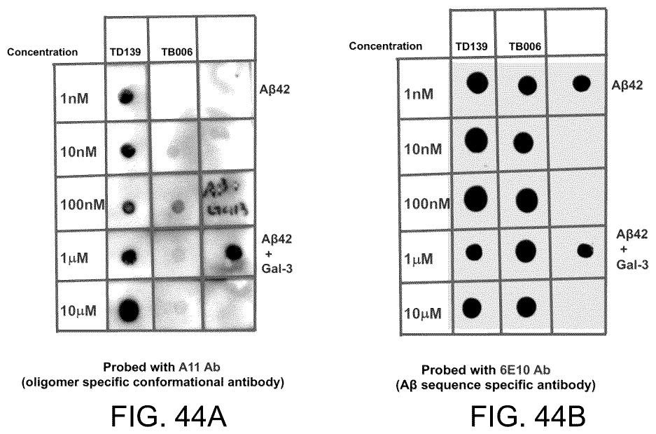

FIG. 44A-B depict comparative degradation of Aβ42 oligomers by TB139 and TB006 incubated at RT and probed with oligomer A11 6E10 degrading antibody as detected by dot blot. FIG. 44A depicts comparative degradation of Aβ42 oligomers by TB139 and TB006 incubated at RT and probed with oligomer A11 degrading antibody as detected by dot blot. FIG. 44B depicts comparative degradation of Aβ42 oligomers by TB139 and TB006 incubated at RT and probed with 6E10 degrading antibody as detected by dot blot.

1 mg of the lyophilized Aβ42 peptide (r-peptide) was resuspended in 90 μl of 100 mM NaOH and incubated for 10 minutes. Solution was then diluted to final concentration 0.1 mg/ml by adding 100 mM sodium phosphate buffer, pH 7.4. Synthetic Aβ42 peptide was mixed with 100 ug of rhGal-3 protein, and incubated for 1 hr at RT. rhGal-3 induces oligomerization. After 1 hr different concentration of TD139 (small molecule from Galecto Inc.) and hTB006Ab (Galectin-3 Antibody from Truebinding Inc.) was added. Solution was incubated for 24 hrs at RT. 2 μl of each sample was pipetted onto a Whatman nitrocellulose membrane for dot blot probed with antibody A11. In order to confirm the Aβ42, membrane was also probed with antibody 6E10. After incubating with respective secondary antibodies for 1 h, images were developed using Azure image developer.

In conclusion TB006 degraded the toxic Aβ42 oligomers while TD139 was not able to degrade these toxic oligomers.

### Example 53: Anti-Gal3 Blocking Antibodies

Anti-Gal3 blocking antibodies were tested to determine the epitopes of hGal3 after binding with TB006 blocking antibodies. Hydrogen/deuterium exchange (HDX) (Trajan, compact) mass spectrometry (MS) (ThermoFisher, Q-Exactive) (HDX-MS) was used to determine the binding site between antibody and antigen. The protein interaction decreases the deuterium uptake at its binding position, and thus causes a mass shift between bound and unbound samples. The mass shift can be detected using mass spec. In a routine HDX-MS analysis, bound and unbound samples are analyzed following a set of optimized parameters. The deuterium uptake difference (bound-unbound) at residue level is demonstrated using a heatmap. In a protein sequence, the residues with negative mass shift indicate that this might be the binding site, and gradient colors, or a greyscale gradient, are commonly used to show the intensity of the binding interaction on different residues in a protein sequence. HDX-MS data is processed using PMI Byos or HDExaminer.

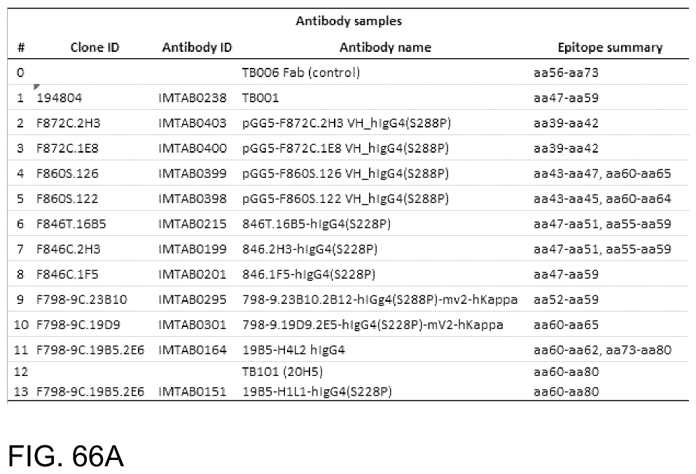

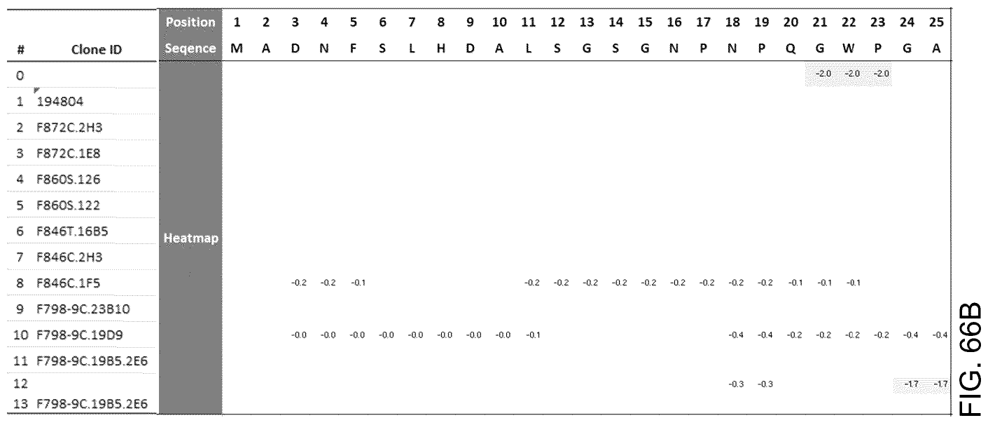

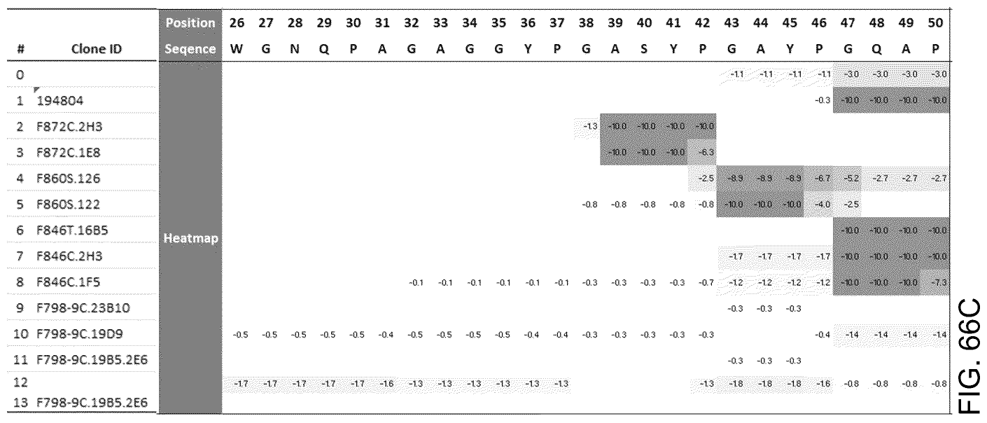

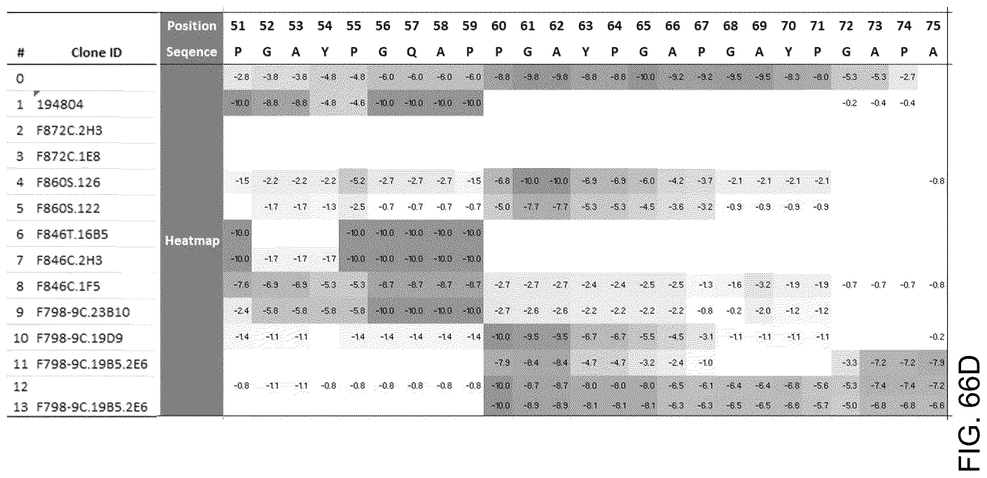

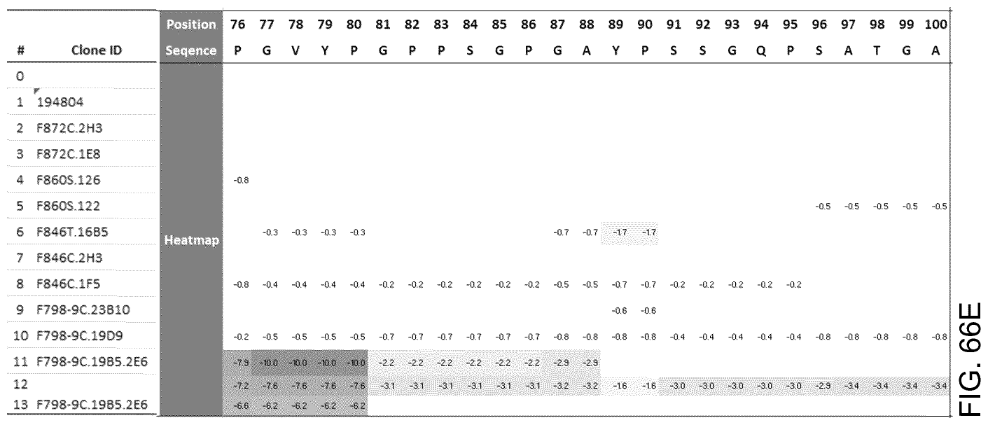

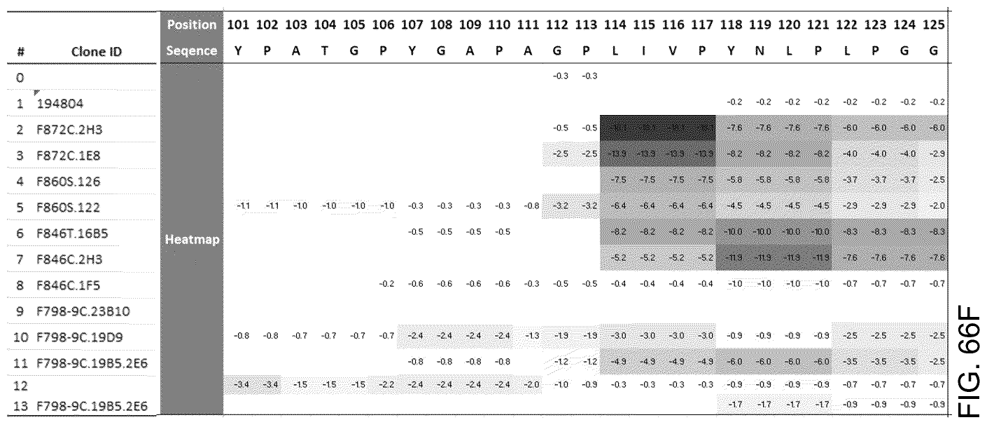

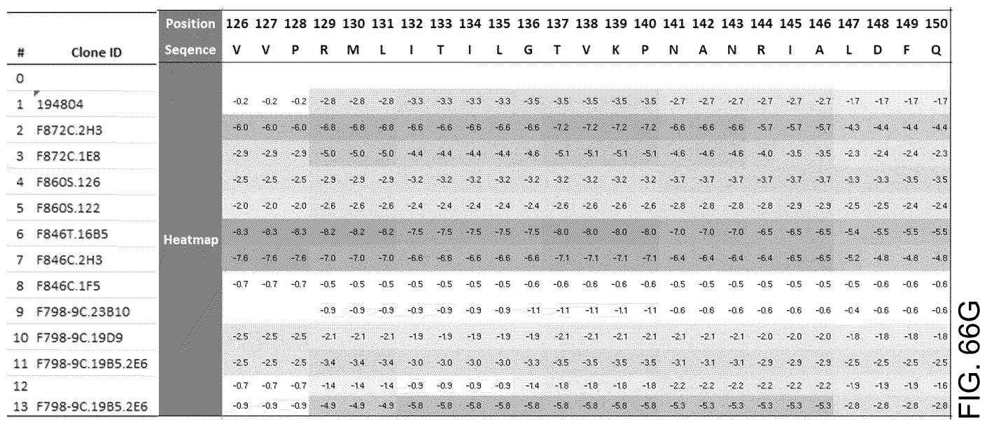

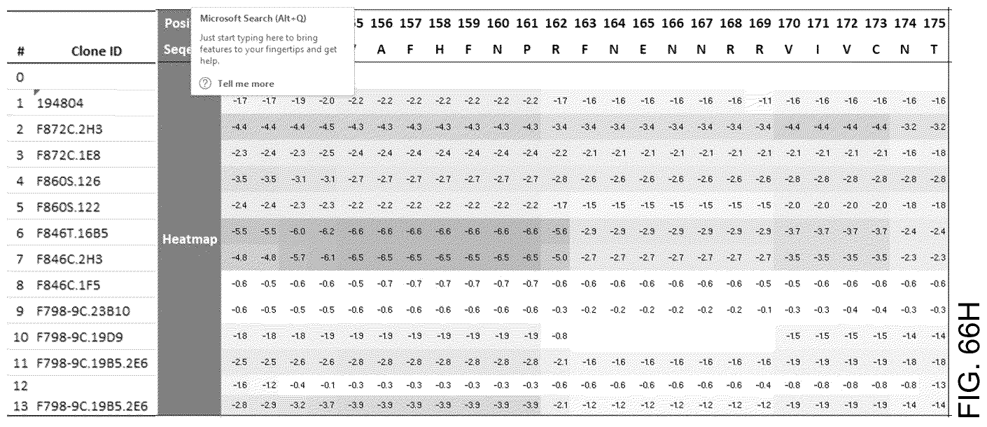

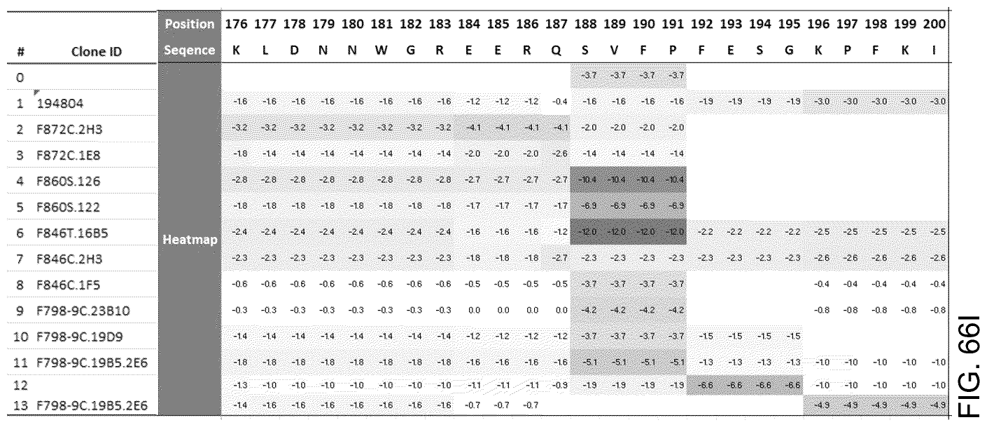

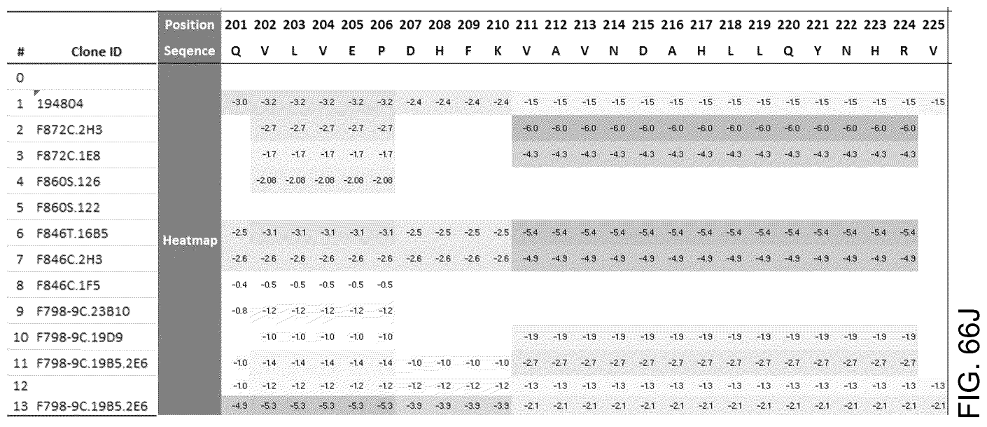

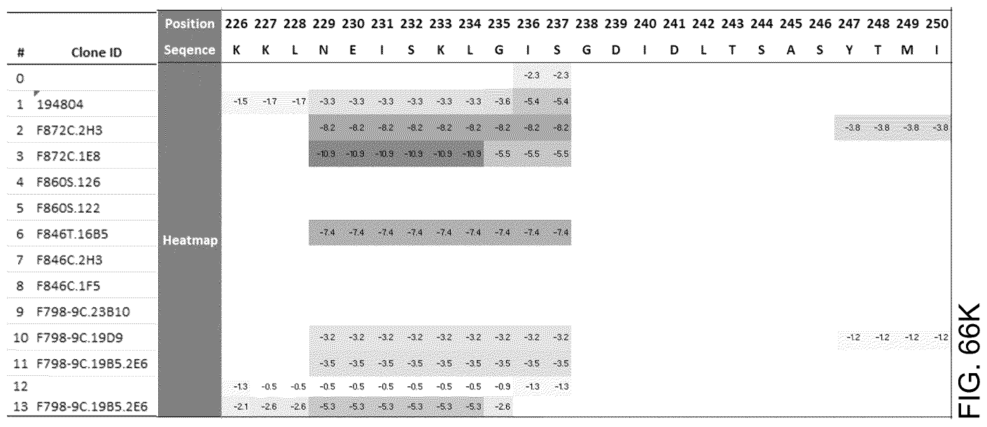

FIG. 66A-k depict heat maps showing the affinity of anti-Gal3 blocking antibodies to various Gal3 residues. FIG. 66A is a table listing some illustrative embodiments of anti-Gal3 blocking antibodies. FIG. 66B-66K depict heat maps showing the affinity of some embodiments of anti-Gal3 blocking antibodies to Gal3 epitopes.

Clean reaction vials were prepared by putting a clean glass insert in a glass vial, and then sealing the glass vial with a new aluminum cap using a clamp. Glass vials can be reused without cleaning. Glass insert can be reused after a thoroughly cleaning. A protease column was then installed in the column chamber of the HDX system. When not in use, a protease column should be stored under 4° C. The nitrogen supply was then checked. A HDX run may consume up to 100 psi nitrogen. The HDX sample outlet tube was connected to a MS ion source and the MS was then configured. A blank buffer was prepared. 1×PBS. 0.5 mL 10×PBS into 4.5 mL milli-Q water Was diluted in a clean HDX buffer vial. The vial was then labeled and stored in HDX plate 1, position H1R1. Expiration was 3 months from the date of preparation. An exchange buffer was prepared. 0.5 mL 10×PBS was diluted into 4.5 mL 99.8% deuterium oxide in a clean HDX buffer vial. The vial was labeled and stored in HDX plate 1, position H1R2. To prepare the quench buffer, 1 mL 8M Guanidine HCl and 0.04 mL 99% formic acid were diluted into 3.96 mL milli-Q water in a clean HDX buffer vial. Expiration was 3 months from the date of preparation. A 1.6M Guanidine HCl with 0.8% Formic acid quench buffer was prepared and stored in HDX plate 2, position H2R1. Expiration was 3 months from the date of preparation. Quench buffer for proteins that don't have disulfide bonds, such as human galectin-3. A protease column wash buffer was prepared. To prepare the protease column wash buffer, 0.563 mL 8M Guanidine HCl and 0.04 mL 99% formic acid were diluted into 4.397 mL milli-Q water in a clean HDX buffer vial. The vials were stored in HDX plate 1, position H1R3. Expiration was 3 months from the date of preparation. LC phase A, 0.1% formic acid in milli-Q water, was prepare by mixing 1 mL 99% formic acid and 999 mL milli-Q water in a 1 L LC bottle. Expiration was 3 months from the date of preparation. LC phase B, 0.1% formic acid in CAN, was prepared by mixing 1 mL 99% formic acid and 999 mL ACN in a 1 L LC bottle. Expiration was 3 months from the date of preparation. LC phase C, 0.1% formic acid in milli-Q water, was prepared by mixing 1 mL 99% formic acid and 999 mL milli-Q water in a 1 L LC bottle. Expiration was 3 months from the date of preparation. LC seal wash buffer, 10% methanol, was prepared by mixing 100 mL methanol and 900 mL milli-Q water in a 1 L LC bottle. Expiration was 3 months from the date of preparation. HDX syringe wash 1, 0.1% formic acid in milli-Q water, was prepared by mixing 1 mL 99% formic acid and 999 mL milli-Q water in a 1 L LC bottle. Expiration was 3 months from the date of preparation. HDX syringe wash 2, 0.1% formic acid in CAN, was prepared by mixing 1 mL 99% formic acid and 999 mL ACN in a 1 L LC bottle. Expiration was 3 months from the date of preparation.

Fresh samples were then prepared for the HDX-MS analysis, the unbound and bound samples were stored in HDX plate 2 sample position 1 and 2, respectively. The bound sample was incubated at room temperature for 1 hour prior to placing it in the HDX plate. In the bound sample, target molecule and its binding pair have a molar ratio 1:5. Expiration was 1 week from the date of preparation.

To determine the binding site of hGal-3, 0.5 mg/mL unbound hGal-3 in 1×PBS buffer and a bound sample comprising 0.5 mg/mL hGal-3 and 7.5 mg/mL antibody in 1×PBS buffer were prepared.

LC parameters consisted of the following: Column: Hypersil Gold column, 50×1 mm, 1.9 um column, Thermo Part #25002-051030; Loading pump pressure upper limit: 250 bar; NC pump pressure upper limit: 620 bar.

MS parameters are listed in Table 2.

TABLE 2

| Parameter | Analysis of hGal-3 | Analysis of antibody or fab |
|---|---|---|
| Method name | HDX_30 min_F4_TandemMS_40 min | HDX_40 minLC_21 minMS_Tandem_TB006Fab.meth |
| Experiment | Full MS −> ddMS² | |
| Method duration | 25 min | 30 min |
| Run time | 1 to 16.5 min | 1 to 21 min |
| Polarity | Positive | |
| Default charge state | 2 | |
| Full MS Resolution | 35,000 | |
| Full MS AGC target | 3e6 | |
| Full MS Maximum IT | 200 ms | |
| Full MS Scan range | 350 to 1750 m/z | |
| dd-MS² Resolution | 17,500 | |
| dd-MS² AGC target | 1e5 | |
| dd-MS² Maximum IT | 50 ms | |
| dd-MS² Loop count | 10 | |
| dd-MS² Top N | 10 | |
| dd-MS² Isolation | 2.0 m/z | |
| window | | |
| dd-MS² (N)CE / Stepped | nce: 27 | |
| (N)CE | | |
| dd settings Minimum | 8.00e3 | |
| AGC target | | |
| dd settings Intensity | 1.6e5 | |
| threshold | | |
| dd settings Charge | Unassigned, >8 | |
| exclusion | | |
| dd settings Peptide match | preferred | |
| dd settings Exclude | on | |
| isotopes | | |
| dd settings Dynamic | 20.0 s | |
| exclusion | | |
| Tune Switch count | 2 | |
| Base tune | HDX tune_CZ_V4_GasSave.mstune | |
| Tune Element 1 at | 1.00 | |
| Element 1 tunefile | HDX tune_CZ_V4.mstune | |
| Tune Element 2 at | 16.50 | 20.98 |

| | |
|---|---|
| Element 2 tunefile | HDX tune_CZ_V4_GasSave.mstune |

HDX parameters are listed in Table 3.

TABLE 3

| Parameter | Analysis of hGal-3 | Analysis of antibody or fab |
|---|---|---|
| Analysis method for blank | Gal-3_H2O.cam | |
| Analysis method for test | Gal-3_D2O.cam | |
| sample | | |
| Exchange time | *4~6 time points from 0 s to a few hours. | |
| | For example: 0, 60, 600, 3600. | |
| Total LC Runtime (min) | 40 | |
| LC backflush Start Time | 30.6 | |
| Protein Asp Vol [μL] | 2.2 | |
| Protein Disp Vol [μL] | 2 | |
| Deuterium Volume [μL] | 18 | |
| Transfer Vol [μL] | 18 | |
| Quench Volume [μL] | 30 | |
| Injection Vol [μL] | 45 | |
| Protein Sample Tray | HDxHolder2: Sample | |
| Protein Sample Position | *Depends on the actual sample position, normally | |
| | unbound sample at position 1 and bound sample at | |
| | position 2. | |
| Quench Reaction Time | 0.5 | |
| Label Plate | HDxHolder 1: Vials | |
| Initial Exchange Vial | *Depends on the position of reaction vials, Such as 1. | |
| Digest Time [s] | 180 | |
| Digest Flow Rate [ml/min] | 0.2 | |
| Pepsin Washes | 2 | |
| Data System Output Dir | *Designate a folder to save data | |
| Data System Method | HDX_30 min_F4_TandemMS_40 min | HDX_40 minLC_21 minMS_Tandem_TB006Fab.meth |

| | |
|---|---|
| Filename | *Distinguish files as blank, unbound, and bound. No |
| | need to specify the exchange time for each file. |

The samples were run and the data were processed. In the data processing, deuterium uptake of hGal3 was normalized to have the upper limit in the N-terminal (aa1-aa112) as 10. The amino acids that have deuterium uptake >5 (half of the strongest binding) were considered to be epitopes.

FIG. 66 depict heat maps showing the affinity of anti-Gal3 blocking antibodies to various Gal3 residues. FIG. 66A is a table depicting some embodiments of anti-Gal3 blocking antibodies. FIG. 66B-66K depict heat maps showing the affinity of some anti-Gal3 blocking antibodies to Gal3 epitopes. HDX results were shown as a heatmap in column G to column IV. Above the heatmap, row 2 and 3 show the amino acid position and sequence of hGal3. Clone #0 (Row 4) shows the HDX results of the control, TB006. #1-12 (row 5-16) shows the data of 12 antibodies that can block TB006>90%.

In the heatmap, black areas indicate binding interactions of hGal3 with the antibodies. The darker the greyscale, the stronger the relative binding interaction. The epitope summary is shown in column E. Binding interactions (dark areas in the heatmap) on hGal3 C-terminal (aa113-aa250) are not considered to be epitopes, because they bind to the glycan instead of the CDRs of an antibody.

Table. 1 list different Gal3 peptides (SEQ ID No.: 1619-1660) used for mapping analysis of TB006, TB101 and 2D10 Ab binding.

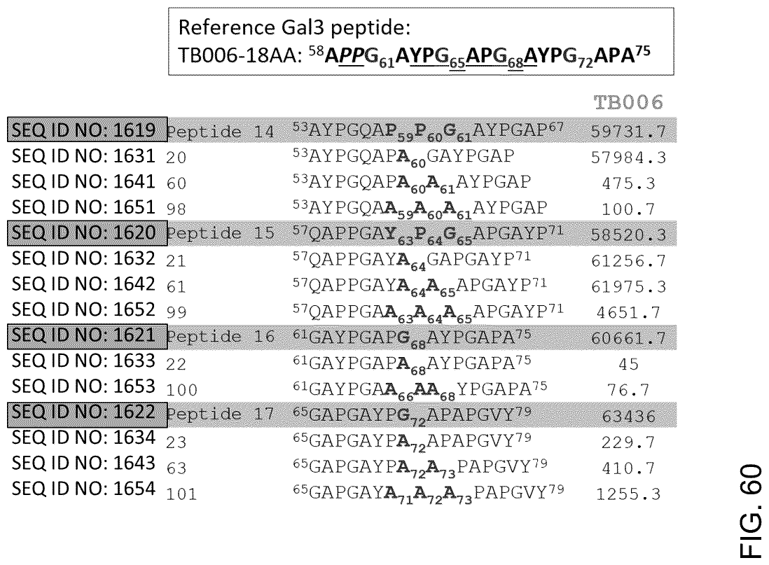

FIG. 60 depicts the mutational analysis of TB006Ab binding with Gal 3 peptides (SEQ ID No. 1619-1630) by Microarray.

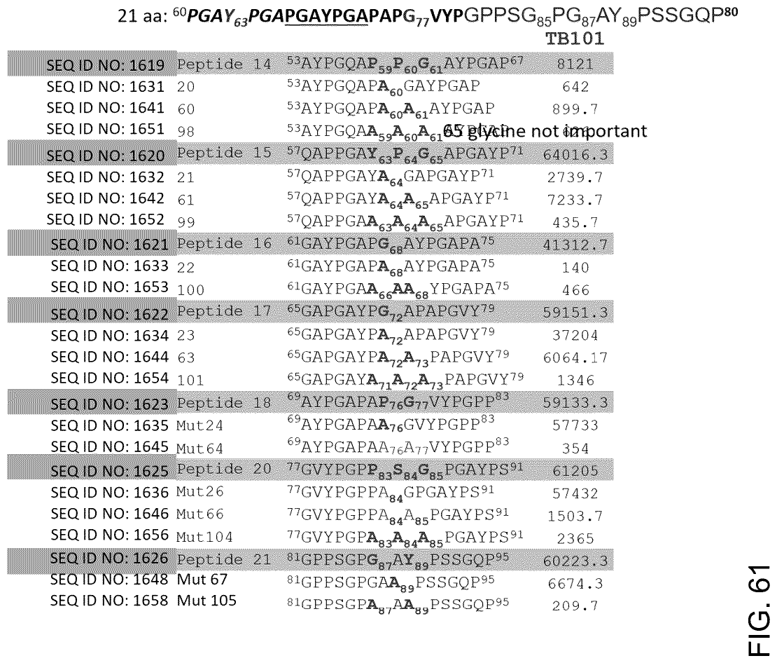

FIG. 61 depicts the mutational analysis of TB101 Ab binding with Gal 3 peptides (SEQ ID No. 1619-1630) by Microarray.

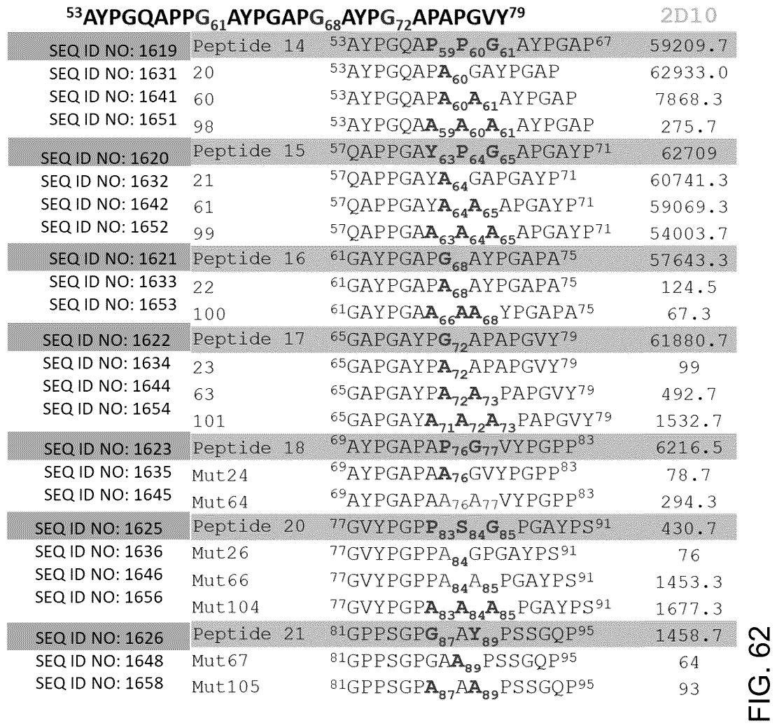

FIG. 62 depicts the mutational analysis of 2D10 Ab binding with Gal 3 peptides (SEQ ID No. 1619-1623, 1631-1634, 1641-1644, and 1651-1654) by Microarray.

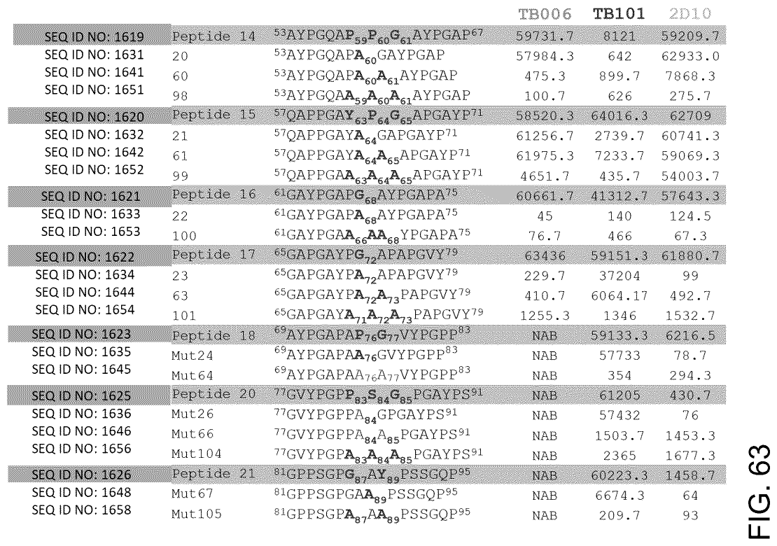

FIG. 63 depicts the mutational analysis of TB006, TB101, 2D10 Ab binding with Gal 3 peptides (SEQ ID No. 1619-1660) by Microarray.

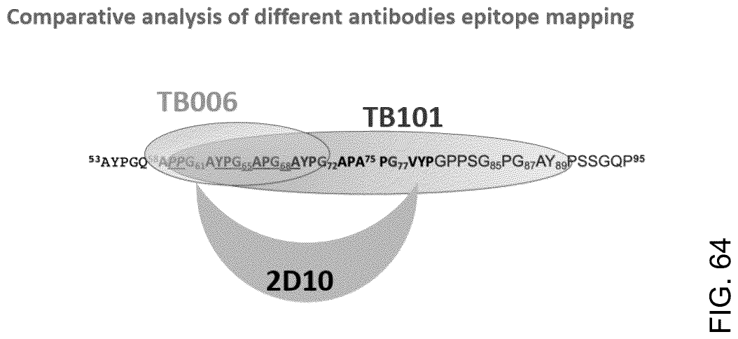

FIG. 64 is an illustrative embodiment showing a comparison of some embodiments of epitope mapping analysis.

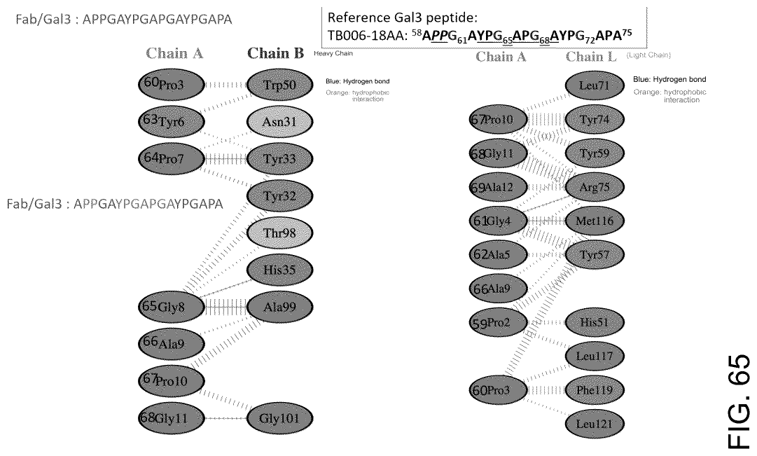

FIG. 65 is an illustrative embodiment showing a comparison of some embodiments of epitope mapping analysis. Chain A represents the amino acid numbers on the 18 aa peptide whereas Chain B represents the amino acid numbers on the Heavy chain of TB006 FAb and Chain L represents the light chain.

As is clear from FIG. 60-66, various anti-Gal3 blocking antibodies block binding of an anti-Gal3 antibody by at least 80% as compared to anti-Gal3 binding in the absence of an anti-Gal3 blocking antibody.

### Example 54: Competition Binding Assay

Anti-Gal3 blocking antibodies were evaluated for their ability to block binding of Gal3 with hTB001, hTB006, Aβ42, Aβ40, Aβ42 alpha synuclein, and hTau. To identify Gal3-targeted antibodies with the ability to block the interaction of Gal3 and Aβ42, human galectin-3 protein (TrueBinding, QCB200375) was diluted 2-fold in PBS from a concentration of 4 μg/ml and coated a 96-well ELISA plate by adding 80 μl per well. After incubating the plate at 4° C. overnight, the plate was washed with 300 μl PBST three times, followed by a blocking step with 150 μl of 2% BSA in PBST per well and incubated for an hour at room temperature (RT) with gentle rocking. The existing blocking solution was then discarded from the plate. Binding solutions were prepared by 2-fold dilutions of Aβ42 from 4 μg/ml in a 2% buffer of BSA in PBST to a concentration of 4 μg/ml. The Aβ42 dilution was then applied to the plate by adding 60 μl per well column-wise for each galectin-3, then serially diluted two-fold length-wise in 2% BSA in PBST. The plate was incubated for an hour at RT with gentle rocking, then washed with 300 μl PBST three times. Afterwards, HRP-tagged anti-FLAG antibodies were diluted to 1:2000 in 2% BSA in PBST, and 25 μl was added to all the wells. The plate was incubated for 40 minutes at RT with gentle rocking, then washed with 300 μl PBST three times. To develop the plate, 50 μl of ABTS substrate was added to each well and incubated until a sufficiently high signal was achieved. The plate was read in a plate reader at an absorbance of 405 nm. Data was graphed using GraphPad Prism 8.0 software (GraphPad Software Inc).

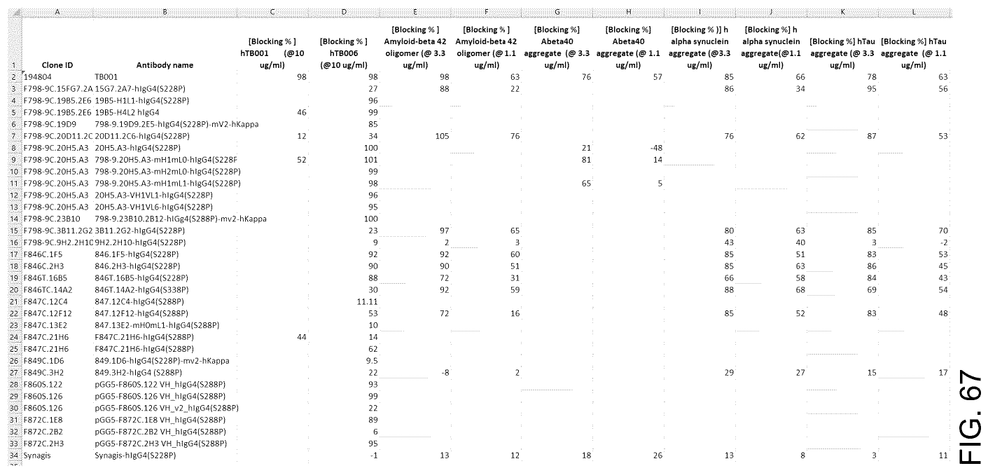

FIG. 67 is a table depicting the ability of 33 different anti-Gal3 blocking antibodies to block Gal3 binding to hTB001, hTB006, Aβ42, Aβ40, Aβ42 alpha synuclein, and hTau.

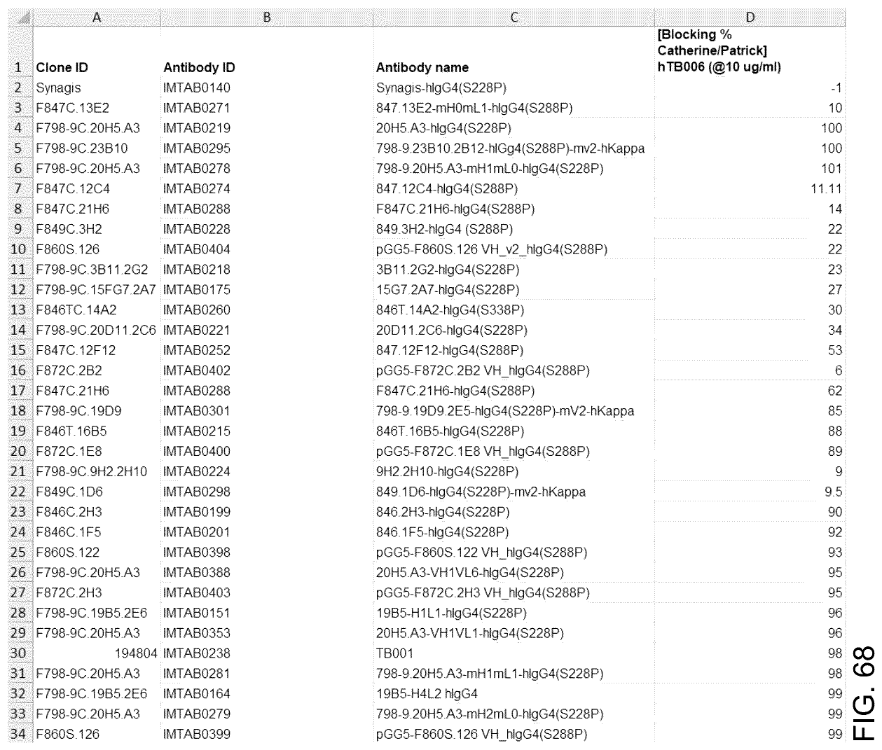

FIG. 68 is a table depicting the ability of 33 different anti-Gal3 blocking antibodies to block Gal3 binding to hTB006 as measured by Elisa. That is, these antibodies compete for binding to Gal3 with hTB006.

As can be seen in FIG. 67-68, many anti-Gal3 blocking antibodies are able to compete with one or more anti-Gal3 antibodies for binding to Gal3, binding to Gal3 with at least 80% efficiency. hTB001, hTB006, Aβ42, Aβ40, Aβ42 alpha synuclein, and hTau.

### Example 55: Protein Crystal Sample Preparation and Data Collection

To determine whether anti-Gal3 blocking antibodies that compete for one or more epitopes as the anti-Gal3 antibodies disclosed herein are suitable for use with the methods of the present disclosure, the crystal structure of hGal3 in complex with TB006 was obtained to determine the binding site between antibody and antigen. XRD is used to solve the proteins 3D structures and determine the binding site between antibody and antigen. Electro density map can be identified when there is stable interaction between antibody and antigen if the protein molecules pack orderly and diffraction resolution is high enough. The distance between two hydrophobic amino acids is in 5 angstroms can be identified as hydrophobic interaction. The distance between two hydrophilic amino acids is within 3.5 angstrom can be identified as hydrogen bond or salt bond interaction. The distance of charged amino acid and aromatic ring of the aromatic amino acid is within 6 angstroms can be identified as cation-π interaction. The distance between two aromatic amino acids is within 4.4 angstroms can be identified as π-π interaction. The XRD data is processed using software suites CCP4, Phenix and COOT. The structure result is demonstrated using software Pymol or Chemira. State-of-art optical microscope (ThermoFisher, Q-Exactive), Liquid nitrogen cylinder (Trajan, compact), Liquid nitrogen storage (Waters, M-Class Acquity), Micro-Tools Set (HAMPTON RESEARCH), Mini-Centrifuge (Sigma-Aldrich), Crystal fishing tools including CrystalCap Systems, CryoLoops, Cryo Tools, Dewars (HAMPTON RESEARCH), Crystal temporary storage pucks and shipping dewars with cooling system (HAMPTON RESEARCH), MilliQ Water (or equivalent).

The protein to be crystallized was concentrated in PBS solution to 30 to 50 mg/mL. The antibody (Ab) was mixed with the antigen (Ag) in a certain molar ratio for Ab-Ag complex crystals. To screen the crystals, an appropriate volume of solutions from the original crystallization condition screen kits into the crystallization plate well. The protein solution and the crystallization solution were then mixed in a 1:1 ratio. Crystallization condition optimization was then performed. The pH value and precipitants or salts concentration were optimized in a certain range. Additives were screened as necessary. Crystal harvesting was then performed by crystal fishing from the drops. The crystals were fished out from the growth drop, soak in the cryo-protectant solution with the gradient increase of the cryo-protectant reagent such as glycerol for different time accordingly. Once the crystal from the last drop of cryo-protectant solution was fished out, the loop with the crystal was dipped into the liquid nitrogen immediately. The crystal was then mounted into the puck or cap. The crystals were diffracted in SSRL or ALS. Data was collected when the crystal was able to diffract to 3 angstroms. The data was transferred data from SSRL or ALS to a local sever and processed using CCP4 software suites to determine the useful resolution. Phoenix was used to determine the phase and build the model. COOT was used to manually adjust the model. The structure was then analyzed using Pymol or Chemira.

### Example 56: Interaction of TB006 and Gal3

To determine whether anti-Gal3 blocking antibodies that compete for one or more epitopes as the anti-Gal3 antibodies disclosed herein are suitable for use with the methods of the present disclosure, the structure of TB006 Fab in complex with hGal3 peptide from amino acid 58 to 75 (58′ APPGAYPGAPGAYPGAPA′75) was determined to 1.95 Å. Purified TB006 Fab from nickel resin was applied to Superdex200 in PBS buffer (137 mM NaCl, 27 mM KCl, 10 mM Na2HPO4 and 1.8 mM KH2PO4).

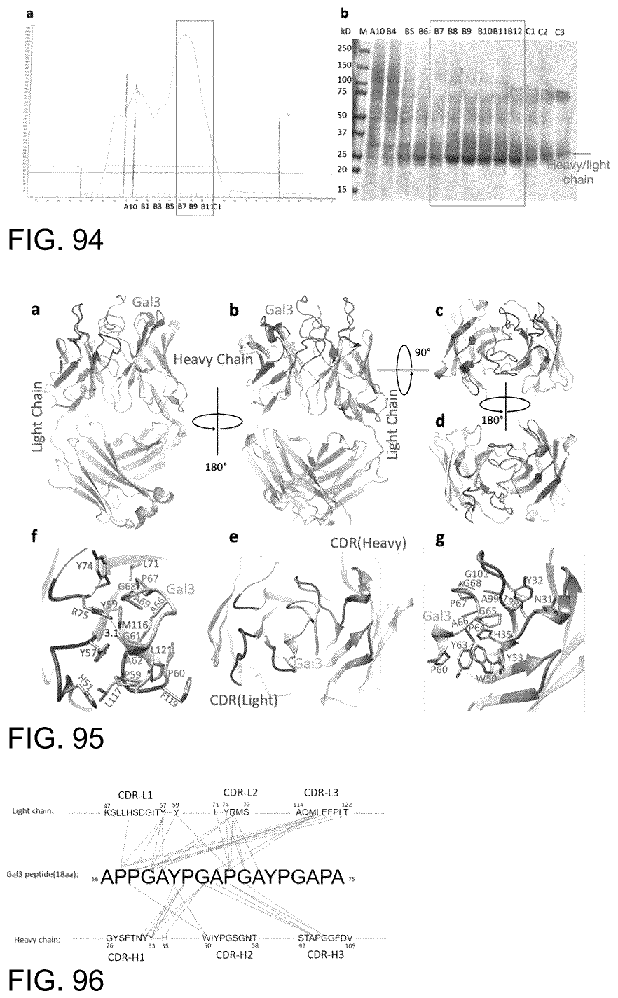

FIG. 94 shows the isolation of TB006 Fab.

Fractions from the main peak (FIG. 94a) with relatively high purity (FIG. 94b) were collected and concentrated to 50 mg/mL when assuming the extinction coefficient as 1. Peptide synthesized by ThermoFisher was solubilized with PBS buffer with additional of 100 mM Tris(hydroxymethyl)aminomethane pH 8.0 to a final concentration of 9 mg/mL. 50 mg/mL TB006 Fab and 9 mg/mL hGal3 peptide were mixed in 1:1 ratio (v/v) to screen crystallization conditions. Crystals were applied to synchrotron radiation source crystallized in condition of 40 mM KH2PO4, 16% (w/v) polyethylene glycerol 8000 and 20% glycerol. Diffraction data was collected at Stanford Synchrotron Radiation Lightsource (SSRL) and processed using CCP4 and Phenix software suites. Coordinate file with PDB code 3pp4 was used as model to search the phase solution using Phenix Phaser M R. Structure model was built use Phenix autobuild and adjusted using COOT and refined using Phenix refine. Structures are illustrated using Pymol or Chemira.

FIG. 95 depicts the crystal structure analysis of TB006 Fab and hGal-3 peptide. FIG. 95a-d depict side view (FIG. 95a and FIG. 95b) and top view (FIG. 95c and FIG. 95d) of overall structure of TB006 Fab in complex with hGal3. Light chain and heavy chain of TB006 Fab are illustrated as depicted in FIG. 95a and FIG. 95b respectively. FIG. 95f and FIG. 95g. depict the interaction interface of TB006 Fab and hGal3 peptide. FIG. 95f depicts detailed amino acids interactions between TB006 Fab light chain and hGal3 peptide. FIG. 95g depicts detailed amino acid interactions between TB006 Fab heavy chain and hGal3 peptide. The black dash indicates hydrogen bond with distance of 3.1 Å. Interactions not highlighted are hydrophobic interaction. Light pink and light blue indicate the light chain and heavy chain respectively. Magentas and marine indicate the CDRs from light chain and heavy chain respectively. hGal3 is showed as green ribbon.

FIG. 96 depicts a summary of interaction between TB006 Fab and hGal-3 peptide. Dot lines indicate the interactions between CDRs and hGal-3 peptide. Residues highlighted are out of CDR frames.

The hGal3 peptide lines in the cavity formed by the CDRs from the light chain and heavy chain (FIG. 94a to 94f). 11 amino acids in the peptide are engaged in the interaction with TB006 Fab. Among them, 8 amino acids which are P59 to A62 and G65 to A69 have interaction with the light chain (FIG. 94f). While 7 amino acids which are P60, Y63 to A69 have interaction with heavy chain (FIG. 94g). Most of the interaction are hydrophobic interaction. The detailed interactions are summarized in FIG. 96, simply H51 and Y57 from CDR-L1, R75 from CDR-L2, M116, L117, E118, F119, P120, and L121 from CDR-L3, N31 and Y32 from CDR-H1, A99 and G101 from CDR-H3 are crucial for the Gal3 peptide binding. In addition, some residues out of the CDR, such as light chain Y59, L71, Y74 and heavy chain Y33, H35, W50, were also shown to have interaction with Gal3 peptide.

### Example 57: Neuroserpin—Fenib Aggregation

To test whether anti-Gal3 antibodies could be utilized to inhibit proteopathies, it was first established using a dot blot that Gal3 is involved in neuroserpin aggregation. Three sets of proteins, 100 μg/ml each of Human Neuroserpin and Gal3 alone or in combinations (Neuroserpin+Gal3), were incubated in phosphate buffer pH 7.4 with continued stirring for the 0-24 hrs time points at room temperature. Oligomerization was assayed using dot blot analysis. 2 μl of each sample was pipetted onto a Whatman nitrocellulose membrane at the appropriate time points (time 0-24 hr) for dot blot probed with conformational oligomer specific antibody A11. After incubating with respective secondary antibodies for 1 h, images were developed using Azure image developer.

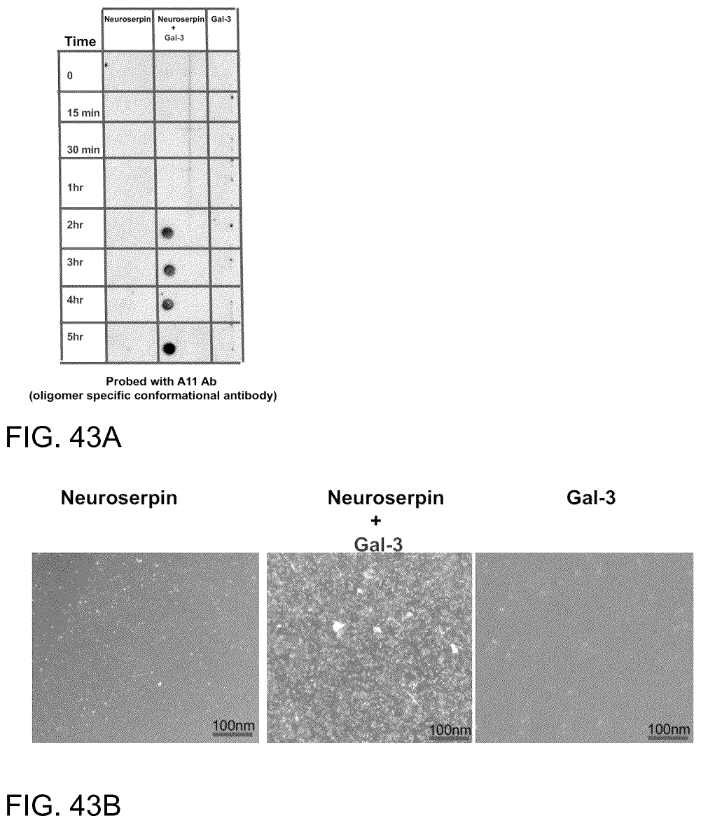

FIG. 43A-B depicts aggregation of neuroserpin incubated at room temperature (RT) with 100 μg of Gal-3 probed with antibody. FIG. 43A depicts 5 hour time course aggregation of neuroserpin as detected by dot blot. FIG. 43B depicts visualization of neuroserpin aggregation with and without Gal-3 as detected using fluorescent microscopy.

As is clear from FIGS. 43A and 43B, Gal3 promotes aggregation of neuroserpin.

### Example 58: Crystallin Aggregation

To test whether anti-Gal3 antibodies could be utilized to inhibit proteopathies, it was first established using a dot blot that Gal3 is involved in crystallin aggregation. Three sets of proteins, 100 μg/ml each of Human Crystallin and Gal3 alone or in combinations (Crystallin+Gal3), were incubated in phosphate buffer pH 7.4 with continued stirring for the 0-24 hrs time points at room temperature. Oligomerization was assayed using dot blot analysis. 2 μl of each sample was pipetted onto a Whatman nitrocellulose membrane at the appropriate time points (time 0-24 hr) for dot blot probed with conformational oligomer specific antibody A11. After incubating with respective secondary antibodies for 1 h, images were developed using Azure image developer.

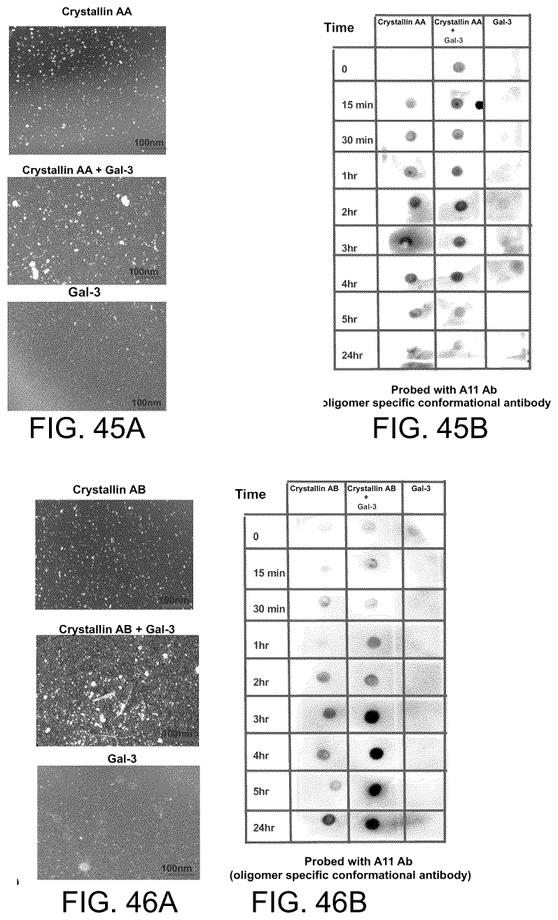

FIG. 45A-B depicts time course aggregation of Crystallin AA incubated with 100 μg of Gal-3 probed with A11 antibody. FIG. 45A depicts visualization of Crystallin AA aggregation with and without Gal-3 as detected using fluorescent microscopy. FIG. 45B depicts time course aggregation of Crystallin AA incubated with 100 μg of Gal-3 probed with A11 antibody as detected by dot blot.

FIG. 46A-B depicts time course aggregation of Crystallin AA incubated with 100 μg of Gal-3 probed with A11 antibody. FIG. 46A depicts visualization of Crystallin AB aggregation with and without Gal-3 as detected using fluorescent microscopy. FIG. 46B depicts time course aggregation of Crystallin AB incubated with 100 μg of Gal-3 probed with A11 antibody as detected by dot blot.

As is clear from FIG. 45-46, Gal3 promotes aggregation of crystallins.

### Example 59: GLUT4-myc Translocation in L6-GLUT4myc Myoblasts

To test whether anti-Gal3 antibodies could be utilized to inhibit proteopathies, it was first established that Insulin-Gal3 aggregates reduce Glucose Transporter-4 translocation in rat L6 GLUT4-myc myoblasts. Prior to start of assay, Gal3-insulin aggregates were prepared. L6 GLUT4-myc myoblasts cells were trypsinized and added to a 96-well plate in MEM a growth media. The plate was incubated at 37 C until 90-95% confluence was reached. The L6 GLUT4-myc myoblasts were serum starved prior to treatments. In total, 3 treatment conditions were tested: 1) Insulin-Gal 3 Aggregates, 2) control buffer, and 3) Insulin alone. Insulin-Gal 3 Aggregates were tested at 30 ng/mL, 100 ng/mL, and 300 ng/mL. Cells were treated with indicated treatments for 1 hr at 370 C. Insulin stimulation was tested in duplicate. At the end of incubation, indicated wells were treated with Insulin for 20 minutes, washed quickly with ice cold DPBS twice, and fixed with paraformaldehyde. Following fixation, the cells were washed three times with PBS, and blocked with goat serum. The cells were then incubated with anti-myc polyclonal antibody. Following incubation, primary antibody was removed by aspiration and washed with 1×DPBS. The cells were then incubated with goat anti rabbit antibody secondary antibody. Following incubation the secondary antibody was removed by aspiration and the cells were washed with PBS. The cells were then incubated with TMB. 1N HCL was added to stop the reaction and the plate was read at 450 nm.

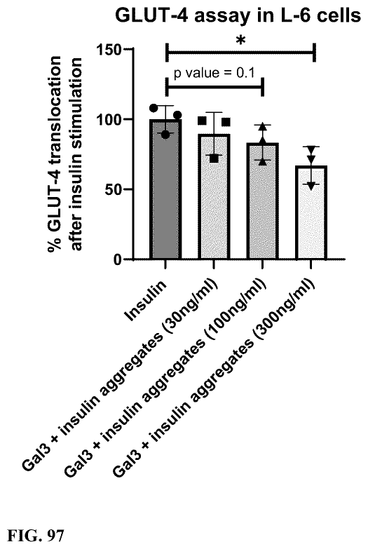

Table 4 depicts the optical density measurements, taken in triplicate, fore each of the treatment conditions. Table 5 depicts the quantification of GLUT4 translocation following insulin stimulation. Table 6 depicts the quantification of the percent GLUT4 translocation following insulin stimulation. FIG. 97 is a bar graph illustrating the percentage stimulation results shown in Table 6.

As is clear from Tables 4-6 and FIG. 97, Gal3+insulin aggregates resulted in reduced GLUT-4 translocation compared to insulin treatment alone.

TABLE 4

| Sr No. | Treatment | OD 1 | OD 2 | OD 3 |
|---|---|---|---|---|
| 1 | Aggregates 300 | 0.1668 | 0.1581 | 0.1589 |
| 2 | Aggregates 300 + Insulin | 0.1217 | 0.1571 | 0.1728 |
| 3 | Aggregates 30 | 0.1442 | 0.1715 | 0.1472 |
| 4 | Aggregates 30 + Insulin | 0.1995 | 0.174 | 0.203 |
| 7 | Aggregates 100 | 0.16 | 0.1423 | 0.1322 |
| 8 | Aggregates 100 + Insulin | 0.1561 | 0.1894 | 0.1577 |
| 9 | Control | 0.1276 | 0.1376 | 0.1224 |
| 10 | Control + Insulin | 0.1926 | 0.1986 | 0.1529 |

TABLE 5

| | | Stimulation | Stimulation | Stimulation |
|---|---|---|---|---|
| Sr No. | Treatment | 1 | 2 | 3 |
| 1 | Aggregates 300 | 0.729616307 | 0.993674889 | 1.0874764 |
| 2 | Aggregates 300 + Insulin | | | |
| 3 | Aggregates 30 | 1.383495146 | 1.014577259 | 1.379076087 |
| 4 | Aggregates 30 + Insulin | | | |
| 7 | Aggregates 100 | 0.975625 | 1.330990864 | 1.192889561 |
| 8 | Aggregates 100 + Insulin | | | |
| 9 | Control | 1.509404389 | 1.443313953 | 1.249183007 |
| 10 | Control + Insulin | | | |

TABLE 6

| | | % | % | % |
|---|---|---|---|---|
| | | Stimulation | Stimulation | Stimulation |
| Sr No. | Treatment | 1 | 2 | 3 |
| 1 | Aggregates 300 | 52.07825175 | 70.9261163 | 77.62144185 |
| 2 | Aggregates 300 + Insulin | | | |
| 3 | Aggregates 30 | 98.75054573 | 72.41807705 | 98.43512398 |
| 4 | Aggregates 30 + Insulin | | | |
| 7 | Aggregates 100 | 69.63775874 | 95.0029168 | 85.14557896 |
| 8 | Aggregates 100 + Insulin | | | |
| 9 | Control | 107.7376437 | 103.0202679 | 89.16366927 |
| 10 | Control + Insulin | | | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of inhibiting Gal3-mediated amyloid aggregation of a protein, comprising:
contacting the protein with an anti-Gal3 antibody or binding fragment thereof,
wherein binding of the anti-Gal3 antibody or binding fragment thereof to Gal3 inhibits Gal3-mediated amyloid aggregation of the protein.

2. A method of inhibiting Gal3-mediated oligomerization of a protein, comprising:
contacting the protein with an anti-Gal3 antibody or binding fragment thereof,
wherein binding of the anti-Gal3 antibody or binding fragment thereof to Gal3 inhibits Gal3-mediated oligomerization of the protein.

3. A method of treating an amyloid proteopathy in a subject in need thereof, comprising:
administering to the subject an anti-Gal3 antibody or binding fragment thereof, wherein
binding of the anti-Gal3 antibody or binding fragment thereof to Gal3 in the subject inhibits Gal3-mediated amyloid aggregation of a protein in the subject, thereby treating the amyloid proteopathy in the subject.

4-36. (canceled)

37. The method of claim 1, wherein Gal3-mediated amyloid aggregation or oligomerization of the protein is inhibited by at least 5%, 10%, 15%, 20%, 25%, 30%, 35%, 40%, 45%, 50%, 55%, 60%, 65%, 70%, 75%, 80%, 85%, 90%, 95%, or 99% after contacting with the anti-Gal3 antibody or binding fragment thereof relative to a cell that is not contacted with the anti-Gal3 antibody or binding fragment thereof.

38. The method of claim 1, wherein the protein comprises α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, and/or any combination thereof.

39. The method of claim 3, wherein identifying the subject as needing treatment of the proteopathy and/or amyloid proteopathy and/or detecting the improvement in the amyloid proteopathy is done by biopsy, blood or urine test, echocardiogram, or technetium pyrophosphate (99mTc-PYP) scintigraphy.

40. The method of claim 3, wherein the proteopathy and/or amyloid proteopathy is reduced by at least 5%, 10%, 15%, 20%, 25%, 30%, 35%, 40%, 45%, 50%, 55%, 60%, 65%, 70%, 75%, 80%, 85%, 90%, 95%, or 99% after the administering step relative to the amyloid proteopathy prior to the administering step.

41. The method of claim 3, wherein the protein comprises α-synuclein, tau protein, TDP-43, transthyretin, uromodulin, islet amyloid polypeptide (IAPP), serum amyloid A (SAA), p53, apolipoprotein E (APOE), APOE-4, prion protein, fibrin, or neurofilament light (NFL), CRP, SUMO, light chain, platelet-derived growth factor receptor (PDGFR), melanoma cell adhesion molecule (MCAM), complement proteins C3 and/or C9, lysozyme, insulin, native haemoglobin (Hb), glycosylated haemoglobin (HbAIC), phenylalanine (Phe), glutamine (Gln), cholesteryl (co-esteryl), cholesterol, neuroserpin, Crystallin AA and/or Crystallin AB, cystatin-C, or myostatin propeptide, and/or any combination thereof.

42. The method of claim 41, wherein the proteopathy and/or amyloid proteopathy comprises a synucleinopathy, Parkinson's disease, dementia with Lewy bodies, multiple system atrophy, tauopathy, Alzheimer's disease, progressive supranuclear palsy, corticobasal degeneration, Pick's disease, TDP-43 proteopathy, amyotrophic lateral sclerosis, frontotemporal lobar degeneration, TTR amyloidosis (ATTR), cardiac amyloidosis, uromodulin-associated kidney disease, IAPP amyloidosis, SAA amyloidosis, rheumatoid arthritis, inflammatory arthritis, spondyloarthropathies, juvenile idiopathic arthritis, ankylosing spondylitis, psoriatic arthritis, inflammatory bowel disease, ulcerative colitis, Crohn's disease, celiac disease, vasculitis, sarcoidosis, familial Mediterranean fever, tumor necrosis factor receptor-associated periodic syndrome (TRAPS), cancer, aging promoted by amyloid aggregation or any combination thereof.

43. The method of claim 1, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 27-70;
the V_{H}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 71-111, 801, 951, 952;
the V_{H}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NO: 112-169, 802, 953, 954;
the V_{L}-CDR1 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 170-220;
the V_{L}-CDR2 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 211-247; and
the V_{L}-CDR3 comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the amino acid sequences of SEQ ID NOs: 248-296; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

44. The method of claim 1, wherein the anti-Gal3 antibody or binding fragment thereof comprises a combination of the V_{H}-CDR1, V_{H}-CDR2, V_{H}-CDR3, V_{L}-CDR1, V_{L}-CDR2, and V_{L}-CDR3 as illustrated in FIG. 13; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

45. The method of claim 1, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 297-373, 803, 806-820, 940, 955-968, 1067-1109, 1415-1439; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

46. The method of claim 1, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 374-447, 821-835, 941-943, 969-982, 1110-1152, 1440-1464; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

47. The method of claim 1, wherein the anti-Gal3 antibody or binding fragment thereof comprises a heavy chain, wherein the heavy chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 448-494, 804, 836-850, 983-996, 1153-1195, 1411, 1465-1489; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

48. The method of claim 1, wherein the anti-Gal3 antibody or binding fragment thereof comprises a light chain, wherein the light chain comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence selected from SEQ ID NOs: 495-538, 805, 851-865, 997-1010, 1196-1238, 1412, 1490-1514; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

49. The method of claim 1, wherein the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.1H2, 847.14H4, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, 849.5H1, 847.23F11, 847.16D10, 847.13E2-mH0mL1, 847.13E2-mH0mL2, 847.12C4, 847.4D3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 20H5.A3-VH3VL1, 20H5.A3-VH3VL3, 20H5.A3-VH4VL1, 20H5.A3-VL5VL1, 20H5.A3-VH5VL3, 20H5.A3-VH6VL1, 20H5.A3-VH6VL3, 2D10-VH0-VL0, 2D10-hVH4-HVL1, 2D10-hVH4-HVL2, 2D10-hVH4-HVL3, 2D10-hVH4-HVL4, 2D10-hVH3-HVL1, 2D10-hVH3-HVL2, 2D10-hVH3-HVL3, 2D10-hVH3-HVL4, 21H6-H0L0, 21H6-H1L1, 21H6-H1L2, 21H6-H1L3, 21H6-H1L4, 21H6-H2L1, 21H6-H2L2, 21H6-H2L3, 21H6-H2L4, 21H6-H3L1, 21H6-H3L2, 21H6-H3L3, 21H6-H3L4, 21H6-H4L1, 21H6-H4L2, 21H6-H4L3, 21H6-H4L4, 21H6-H5L1, 21H6-H5L2, 21H6-H5L3, 21H6-H5L4, 21H6-H6L1, 21H6-H6L2, 21H6-H6L3, 21H6-H6L4 or binding fragment thereof; or wherein the antibody or binding fragment thereof comprises a blocking antibody that competes for binding with any one or more of the preceding anti-Gal3 antibodies or binding fragments thereof, wherein the blocking antibody is at least 80% effective at outcompeting the anti-Gal3 antibody or binding fragment thereof.

50. The method of claim 43, wherein 80% effectiveness at outcompeting the anti-Gal3 antibody or binding fragment thereof is determined as follows:
A) Ab diluted 2-fold in PBS from a concentration of 4 μg/ml and coated a 96-well ELISA plate by adding 80 μl per well;
B) After incubating the plate at 4° C. overnight, the plate is washed with 300 μl PBST three times, followed by a blocking step with 150 μl of 2% BSA in PBST per well and incubated for an hour at room temperature (RT) with gentle rocking;
C) Binding solutions are prepared by 2-fold dilutions from 4 μg/ml in a 2% buffer of BSA in PBST to a concentration of 4 μg/ml;
D) The dilution is then applied to the plate by adding 60 μl per well column-wise for each galectin-3, then serially diluted two-fold length-wise in 2% BSA in PBST;
E) The plate is incubated for an hour at RT with gentle rocking, then washed with 300 μl PBST three times;
F) Afterwards, HRP-tagged anti-FLAG antibodies are diluted to 1:2000 in 2% BSA in PBST, and 25 μl is added to all the wells
G) The plate is incubated for 40 minutes at RT with gentle rocking, then washed with 300 μl PBST three times;
H) To develop the plate, 50 μl of ABTS substrate is added to each well and incubated until a sufficiently high signal was achieved;
I) The plate is read in a plate reader at an absorbance of 405 nm; and optionally,
J) Data can be graphed using GraphPad Prism 8.0 software (GraphPad Software Inc).

51. The method of claim 1, wherein the protein is selected from amyloid β40 and amyloid β42.

52. The method of claim 1, wherein the protein is selected from alpha synuclein, APOE-4, cholesterol, insulin, IAPP, transthyretin, crystallins, atrial natriuretic peptide (ANP) and B-Type Natriuretic Peptide (BNP).
